(19) United States
(12) Patent Application Publication
(10) Pub. No.: US 2024/0026392 A1
(43) Pub. Date: Jan. 25, 2024

(54) BIOSYNTHESIS OF CANNABINOIDS AND CANNABINOID PRECURSORS

(71) Applicant: Ginkgo Bioworks, Inc., Boston, MA (US)

(72) Inventors: Kim Cecelia Anderson, Somerville, MA (US); Jeffrey Ian Boucher, Allston, MA (US); Elena Brevnova, Winchester, MA (US); Brian Carvalho, Somerville, MA (US); Nicholas Flores, Cambridge, MA (US); Katrina Forrest, Seattle, WA (US); Fiona Qu, Boston, MA (US); Gabriel Rodrigues, Central, SC (US); Michelle Spencer, Boston, MA (US)

(73) Assignee: Ginkgo Bioworks, Inc., Boston, MA (US)

(21) Appl. No.: 18/015,046

(22) PCT Filed: Jul. 8, 2021
(86) PCT No.: PCT/US2021/040941

§ 371 Date: Jan. 6, 2023

Related U.S. Application Data
(60) Provisional application No. 63/067,840, filed on Aug. 19, 2020.
(60) Provisional application No. 63/049,546, filed on Jul. 8, 2020.

(51) Int. Cl. C12P 7/42; C12P 7/22; C12N 15/81

(52) U.S. Cl. CPC C12P 7/42; C12P 7/22; C12N 15/81; C12Y 121/03008; C12Y 404/01026; C12Y 205/0101; C12Y 203/01206; C12N 2800/102

(57) ABSTRACT

Aspects of the disclosure relate to biosynthesis of cannabinoids and cannabinoid precursors in recombinant cells and in vitro, and to variant terminal synthases having altered catalytic activity to synthesize THC-type, CBD-type and/or CBC-type cannabinoids.

### CROSS REFERENCE TO RELATED APPLICATION

This application claims the benefit under 35 U.S.C. § 119(e) of U.S. Provisional Application No. 63/049,546 filed Jul. 8, 2020, entitled “BIOSYNTHESIS OF CANNABINOIDS AND CANNABINOID PRECURSORS,” and U.S. Provisional Application No. 63/067,840 filed Aug. 19, 2020, entitled “BIOSYNTHESIS OF CANNABINOIDS AND CANNABINOID PRECURSORS,” the entire disclosure of each of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCH format via EFS-Web and is hereby incorporated by reference in its entirety. The ASCII file, created on Jul. 8, 2021, is named G091970065WO00-SEQ-EAS.txt and is 3,909,600 bytes in size.

### FIELD OF INVENTION

The present disclosure relates to the biosynthesis of cannabinoids and cannabinoid precursors, such as in recombinant cells.

### BACKGROUND

Cannabinoids are chemical compounds that may act as ligands for endocannabinoid receptors and have multiple medical applications. Traditionally, cannabinoids have been isolated from plants of the genus Cannabis. The use of plants for producing cannabinoids is inefficient, however, with isolated products often limited to the two most prevalent endogenous cannabinoids, THC and CBD, as other cannabinoids are typically produced in very low concentrations in Cannabis plants. Further, the cultivation of Cannabis plants is restricted in many jurisdictions. In addition, in order to obtain consistent results, Cannabis plants are often grown in a controlled environment, such as indoor grow rooms without windows, to provide flexibility in modulating growing conditions such as lighting, temperature, humidity, airflow, etc. Growing Cannabis plants in such controlled environments can result in high energy usage per gram of cannabinoid produced, especially for rare cannabinoids that the plants produce only in small amounts. For example, lighting in such grow rooms is provided by artificial sources, such as high-powered sodium lights. As many species of Cannabis have a vegetative cycle that requires 18 or more hours of light per day, powering such lights can result in significant energy expenditures. It has been estimated that between 0.88-1.34 kWh of energy is required to produce one gram of THC in dried Cannabis flower form (e.g., before any extraction or purification). Additionally, concern has been raised over agricultural practices in certain jurisdictions, such as California, where the growing season coincides with the dry season such that the water usage may impact connected surface water in streams (Dillis, Christopher, Connor McIntee, Van Butsic, Lance Le, Kason Grady, and Theodore Grantham. “Water storage and irrigation practices for cannabis drive seasonal patterns of water extraction and use in Northern California.” Journal of Environmental Management 272 (2020): 110955).

Cannabinoids can be produced through chemical synthesis (see, e.g., U.S. Pat. No. 7,323,576 to Souza et al). However, such methods suffer from low yields and high cost. Production of cannabinoids, cannabinoid analogs, and cannabinoid precursors using engineered organisms may provide an advantageous approach to meet the increasing demand for these compounds.

### SUMMARY

Aspects of the present disclosure provide methods for production of cannabinoids and cannabinoid precursors from fatty acid substrates using genetically modified host cells.

Aspects of the disclosure relate to host cells that comprise a heterologous polynucleotide encoding a terminal synthase (TS), wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14, and wherein the TS is capable of producing a THC-type cannabinoid.

In some embodiments, relative to the sequence of SEQ ID NO: 14, the TS further comprises an amino acid substitution at one or more residues corresponding to positions 31, 40, 41, 46, 49, 51, 56, 59, 61, 63, 74, 90, 96, 100, 103, 116, 143, 173, 196, 250, 257, 290, 296, 311, 354, 377, 378, 411, 417, 446, 494, 495, 528, 542, 543 and/or 544 in SEQ ID NO: 14. In some embodiments, the TS is capable of producing more of a THC-type cannabinoid than a control TS, wherein the control TS comprises the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

Further aspects of the disclosure relate to host cells that comprise a heterologous polynucleotide encoding a TS, wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396,411, 417, 419, 424, 443, 446,459, 462, 464, 469, 479, 475, 491, 492, 494, 495, 499, 528, 542, 543, and/or 544 in SEQ ID NO: 14, wherein the TS does not comprise SEQ ID NO: 20, 21, 320 or 321, wherein the TS is capable of producing more of a THC-type cannabinoid than a control TS, and wherein the control TS comprises the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

In some embodiments, the THC-type cannabinoid is tetrahydrocannabinolic acid (THCA) and/or tetrahydrocannabivarinic acid (THCVA). In some embodiments, the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a THC-type cannabinoid than a control TS, wherein the control TS comprises the sequence of SEQ ID NO: 284 or SEQ ID NO: 21. In some embodiments, the TS is capable of producing at least 1, 2, 3, or 4-fold more of a THC-type cannabinoid than a control TS, wherein the control TS comprises the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

In some embodiments, the TS comprises: the amino acid Q at a residue corresponding to position 31 in SEQ ID NO: 14; the amino acid H or Q at a residue corresponding to position 36 in SEQ ID NO: 14; the amino acid E or Q at a residue corresponding to position 40 in SEQ ID NO: 14; the amino acid Y at a residue corresponding to position 41 in SEQ ID NO: 14; the amino acid T at a residue corresponding to position 44 in SEQ ID NO: 14; the amino acid A or P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid P or S at a residue corresponding to position 58 in SEQ ID NO: 14; the amino acid F at a residue corresponding to position 59 in SEQ ID NO: 14; the amino acid S at a residue corresponding to position 61 in SEQ ID NO: 14; the amino acid L or V at a residue corresponding to position 63 in SEQ ID NO: 14; the amino acid T at a residue corresponding to position 74 in SEQ ID NO: 14; the amino acid N at a residue corresponding to position 76 in SEQ ID NO: 14; the amino acid I at a residue corresponding to position 85 in SEQ ID NO: 14; the amino acid L at a residue corresponding to position 88 in SEQ ID NO: 14; the amino acid D, E, or H at a residue corresponding to position 89 in SEQ ID NO: 14; the amino acid E or V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid A at a residue corresponding to position 100 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid Q at a residue corresponding to position 150 in SEQ ID NO: 14; the amino acid L at a residue corresponding to position 158 in SEQ ID NO: 14, the amino acid A at a residue corresponding to position 173 in SEQ ID NO: 14; the amino acid A at a residue corresponding to position 181 in SEQ ID NO: 14; the amino acid K at a residue corresponding to position 196 in SEQ ID NO: 14; the amino acid D at a residue corresponding to position 211 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid D or P at a residue corresponding to position 250 in SEQ ID NO: 14; the amino acid V at a residue corresponding to position 255 in SEQ ID NO: 14; the amino acid M or R at a residue corresponding to position 257 in SEQ ID NO: 14; the amino acid N at a residue corresponding to position 267 in SEQ ID NO: 14: the amino acid E at a residue corresponding to position 268 in SEQ ID NO: 14; the amino acid V at a residue corresponding to position 273 in SEQ ID NO: 14; the amino acid H at a residue corresponding to position 274 in SEQ ID NO: 14; the amino acid L, M, or T at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid Q at a residue corresponding to position 329 in SEQ ID NO: 14; the amino acid E at a residue corresponding to position 340 in SEQ ID NO: 14; the amino acid Q at a residue corresponding to position 344 in SEQ ID NO: 14; the amino acid L or M at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid K at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid V at a residue corresponding to position 417 in SEQ ID NO: 14; the amino acid F at a residue corresponding to position 419 in SEQ ID NO: 14; the amino acid D at a residue corresponding to position 424 in SEQ ID NO: 14; the amino acid I at a residue corresponding to position 443 in SEQ ID NO: 14; the amino acid I at a residue corresponding to position 446 in SEQ ID NO: 14; the amino acid L at a residue corresponding to position 459 in SEQ ID NO: 14; the amino acid I at a residue corresponding to position 462 in SEQ ID NO: 14: the amino acid N at a residue corresponding to position 464 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid M at a residue corresponding to position 491 in SEQ ID NO: 14; the amino acid N at a residue corresponding to position 492 in SEQ ID NO: 14; the amino acid D, E, F, or P at a residue corresponding to position 494 in SEQ ID NO: 14; the amino acid E or K at a residue corresponding to position 495 in SEQ ID NO: 14; the amino acid Q at a residue corresponding to position 499 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14, and/or the amino acid R at a residue corresponding to position 544 in SEQ ID NO: 14.

In some embodiments, the TS comprises: the amino acid H or Q at a residue corresponding to position 36 in SEQ ID NO: 14; the amino acid T at a residue corresponding to position 44 in SEQ ID NO: 14; the amino acid T at a residue corresponding to position 47 in SEQ ID NO: 14; the amino acid I at a residue corresponding to position 52 in SEQ ID NO: 14; the amino acid P or S at a residue corresponding to position 58 in SEQ ID NO: 14; the amino acid I at a residue corresponding to position 85 in SEQ ID NO: 14; the amino acid L at a residue corresponding to position 88 in SEQ ID NO: 14; the amino acid D, E, or H at a residue corresponding to position 89 in SEQ ID NO: 14; the amino acid G at a residue corresponding to position 95 in SEQ ID NO: 14; the amino acid I at a residue corresponding to position 129 in SEQ ID NO: 14; the amino acid R at a residue corresponding to position 136 in SEQ ID NO: 14; the amino acid Q at a residue corresponding to position 150 in SEQ ID NO: 14; the amino acid L at a residue corresponding to position 158 in SEQ ID NO: 14; the amino acid A at a residue corresponding to position 181 in SEQ ID NO: 14; the amino acid D at a residue corresponding to position 211 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid V at a residue corresponding to position 255 in SEQ ID NO: 14; the amino acid N at a residue corresponding to position 267 in SEQ ID NO: 14, the amino acid E at a residue corresponding to position 268 in SEQ ID NO: 14; the amino acid V at a residue corresponding to position 273 in SEQ ID NO: 14; the amino acid H at a residue corresponding to position 274 in SEQ ID NO: 14; the amino acid L, M, or T at a residue corresponding to position 288 in SEQ ID NO: 14; the amino acid Q at a residue corresponding to position 302 in SEQ ID NO: 14; the amino acid I at a residue corresponding to position 309 in SEQ ID NO: 14; the amino acid L at a residue corresponding to position 318 in SEQ ID NO: 14; the amino acid Q at a residue corresponding to position 329 in SEQ ID NO: 14; the amino acid E at a residue corresponding to position 340 in SEQ ID NO: 14; the amino acid Q at a residue corresponding to position 344 in SEQ ID NO: 14; the amino acid L or M at a residue corresponding to position 345 in SEQ ID NO: 14; the amino acid I at a residue corresponding to position 351 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid A at a residue corresponding to position 379 in SEQ ID NO: 14; the amino acid K at a residue corresponding to position 382 in SEQ ID NO: 14; the amino acid V at a residue corresponding to position 396 in SEQ ID NO: 14; the amino acid F at a residue corresponding to position 419 in SEQ ID NO: 14; the amino acid D at a residue corresponding to position 424 in SEQ ID NO: 14; the amino acid I at a residue corresponding to position 443 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid M at a residue corresponding to position 469 in SEQ ID NO: 14; the amino acid M at a residue corresponding to position 479 in SEQ ID NO: 14; the amino acid M at a residue corresponding to position 491 in SEQ ID NO: 14; the amino acid N at a residue corresponding to position 492 in SEQ ID NO: 14; and/or the amino acid Q at a residue corresponding to position 499 in SEQ ID NO: 14.

In some embodiments, the TS comprises at least 2, 3, 4, 5, 6, 7, 8, 9, 10, 11, 12, 13, 14, 15, or 16 amino acid substitutions at residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14. In some embodiments, the TS comprises at least 2, 3, 4, 5, 6, 7, 8, 9, 10, 11, 12, 13, 14, 15, or 16 amino acid substitutions at residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14.

In some embodiments, the TS comprises relative to SEQ ID NO: 14: R31Q, H56N, Q58S, M61 S, 174T, N90V, H143E, A250P, S255V, V288L, T340E, F345L, E424D, Q475K, T492N, and P542L; R31Q, V52I, H56N, Q58S, M61S, I74T, N90V, A250P, S255V, V288L, F345L, Q475K, and T492N; R31Q, A47T, V52I, H56N, Q58S, M61S, I74T, N90V, H143E, A250P, S255V, V288L, T340E, F345L, Q475K, and T492N; H56N, Q58S, M61S, I74T, N90V, H143E, A250D, S255V, V288L, F345L, Q475K, T492N, and A495E; R31Q, H56N, Q58S, M61S, I74T, N90V, H143E, A250P, S255V, V288L, Q475K, and T492N; R31Q, A47T, H56N, Q58S, M61S, I74T, N90V, H143E, A250P, S255V, V288L, T340E, F345L, E424D, Q475K, and T492N; A47T, H56N, Q58S, M61S, I74T, N90V, A250D, S255V, F345L, E424D, Q475K, and T492N; R31Q, A47T, H56N, Q58S, M61S, I74T, N90V, A250P, S255V, V288L, T340E, F345L, Q475K, and T492N; R31Q, A47T, H56N, Q58S, M61S, 174T, N90V, H143E, A250P, S255V, V288L, T340E, F345L, Q475K, and T492N; A47T, H56N, Q58S, M61S, I74T, N90V, A250D, S255V, F345L, Q475K, and T492N; R31Q, A47T, H56N, Q58S, M61 S, 174T, N90V, H143E, A250P, S255V, T340E, F345L, Q475K, and T492N; R31Q, H56N, Q58S, M61S, I74T, N90V, A250P, S255V, V288L, F345L, E424D, Q475K, and T492N; R31Q, A47T, H56N, Q58S, M61S, I74T, N90V, A250P, S255V, T340E, F345L, E424D, Q475K, and T492N; A47T, H56N, Q58S, M61S, I74T, N90V, A250D, S255V, V288L, F345L, Q475K, and T492N; H56N, Q58S, M61S, I74T, N90V, H143E, A250D, S255V, V288L, F345L, Q475K, and T492N; or R31Q, V52I, H56N, M61S, I74T, N90V, A250P, S255V, F345L, Q475K, and T492N.

In some embodiments, the TS comprises relative to SEQ ID NO: 14: M61S, N90V, A250D, S255V, Q475K, T492N, and A495E; H56N, M61S, 174T, N90V, A250P, S255V, T492N, and H494E; or R31Q, H56N, I74T, N90V, A250P, S255V, Q475K, T492N, H494E, and A495E.

In some embodiments, the TS comprises a sequence that is at least 90%, at least 95%, at least 97%, at least 98%, or at least 99% identical to any one of SEQ ID NOs: 505, 563, or 560. In some embodiments, the TS comprises a sequence that is at least 90%, at least 95%, at least 97%, at least 98%, or at least 99% identical to any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913, or a conservatively substituted version thereof. In some embodiments, the TS comprises a sequence that is at least 90%, at least 95%, at least 97%, at least 98%, or at least 99% identical to any one of SEQ ID NOs: 711, 713, 715, 718, 719, 724, 726, 733, 734, 741, 765, 884, 885, 890, 891, and 900, or a conservatively substituted version thereof. In some embodiments, the TS comprises the sequence of any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913, or a conservatively substituted version thereof.

Further aspects of the disclosure relate to host cells that comprise a heterologous polynucleotide encoding a TS, wherein the TS comprises: a sequence that is at least 97% identical to SEQ ID NO: 40; a sequence that is at least 98% identical to any one of SEQ ID NO: 37, 39, and 42; a sequence that is at least 99% identical to SEQ ID NO: 43; or a sequence comprising SEQ ID NO: 38; wherein the host cell is capable of producing a THC-type cannabinoid. In some embodiments, THC-type cannabinoid is THCA and/or THCVA. In some embodiments, the TS is capable of producing more of a THC-type cannabinoid than a control TS, wherein the control TS comprises the sequence of SEQ ID NO: 284 or SEQ ID NO: 21. In some embodiments, the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a THC-type cannabinoid than a control TS, wherein the control TS comprises the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

In some embodiments, the TS further comprises a first signal peptide. In some embodiments, the first signal peptide comprises SEQ ID NO: 16 or a sequence that has no more than two amino acid substitutions, insertions, additions, or deletions relative to the sequence of SEQ ID NO: 16. In some embodiments, the first signal peptide is located at the amino terminus of the TS. In some embodiments, a methionine residue is added to the N-terminus of SEQ ID NO: 16. In some embodiments, the TS further comprises a second signal peptide. In some embodiments, the second signal peptide comprises SEQ ID NO: 17 or a sequence that has no more than one amino acid substitution, insertion, addition, or deletion relative to the sequence of SEQ ID NO: 17. In some embodiments, the second signal peptide is located at the carboxyl terminus of the TS.

In some embodiments, the host cell further produces one or more of cannabidiolic acid (CBDA), cannabidivarinic acid (CBDVA), cannabichromenic acid (CBCA) and/or cannabichromevarinic acid (CBCVA). In some embodiments, the TS produces a higher ratio of THCA:CBDA, THCA:CBCA, THCVA:CBDVA and/or THCVA:CBCVA than a control TS. In some embodiments, the control TS is a TS comprising the sequence of SEQ ID NO: 284 or SEQ ID NO: 21. In some embodiments, the TS has a higher product specificity for a THC-type cannabinoid than a control TS. In some embodiments, the control TS is a TS comprising the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

Further aspects of the disclosure relate to host cells that comprise a heterologous polynucleotide encoding a TS, wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13, wherein the TS is capable of producing a CBD-type cannabinoid.

In some embodiments, relative to the sequence of SEQ ID NO: 13, the TS further comprises an amino acid substitution at one or more residues corresponding to positions 31, 47, 49, 50, 56, 57, 58, 69, 89, 95, 100, 103, 116, 124, 143, 162, 167, 168, 171, 172, 175, 180, 196, 213, 250, 287, 343, 344, 376, 377, 378, 394, 410, 414, 415, 445, 490, 492, 517 and/or 542 in SEQ ID NO: 13. In some embodiments, the TS is capable of producing more of a CBD-type cannabinoid than a control TS, wherein the control TS comprises the sequence of SEQ ID NO. 136.

Further aspects of the disclosure relate to host cells that comprise a heterologous polynucleotide encoding a TS, wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168, 171,172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13, wherein the TS is capable of producing more of a CBD-type cannabinoid than a control TS, and wherein the control TS comprises the sequence of SEQ ID NO: 136.

In some embodiments, the CBD-type cannabinoid is CBDA and/or CBDVA. In some embodiments, the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBD-type cannabinoid than a control TS, wherein the control TS comprises the sequence of SEQ ID NO: 136. In some embodiments, the TS is capable of producing at least 1, 2, 3, or 4-fold more of a CBD-type cannabinoid than a control TS, wherein the control TS comprises the sequence of SEQ ID NO: 136.

In some embodiments, the TS comprises: the amino acid Q at a residue corresponding to position 31 in SEQ ID NO: 13; the amino acid A at a residue corresponding to position 47 in SEQ ID NO: 13; the amino acid P at a residue corresponding to position 49 in SEQ ID NO: 13; the amino acid N at a residue corresponding to position 50 in SEQ ID NO: 13; the amino acid H at a residue corresponding to position 56 in SEQ ID NO: 13; the amino acid D at a residue corresponding to position 57 in SEQ ID NO: 13; the amino acid Q at a residue corresponding to position 58 in SEQ ID NO: 13, the amino acid R or Q at a residue corresponding to position 69 in SEQ ID NO: 13; the amino acid G at a residue corresponding to position 79 in SEQ ID NO: 13; the amino acid N, D, E, Q, or R at a residue corresponding to position 89 in SEQ ID NO. 13; the amino acid C at a residue corresponding to position 90 in SEQ ID NO: 13; the amino acid A at a residue corresponding to position 95 in SEQ ID NO: 13; the amino acid A at a residue corresponding to position 100 in SEQ ID NO: 13; the amino acid H at a residue corresponding to position 103 in SEQ ID NO: 13; the amino acid E at a residue corresponding to position 106 in SEQ ID NO: 13, the amino acid A or G at a residue corresponding to position 116 in SEQ ID NO: 13; the amino acid N or M at a residue corresponding to position 124 in SEQ ID NO: 13; the amino acid E at a residue corresponding to position 143 in SEQ ID NO: 13; the amino acid Q at a residue corresponding to position 150 in SEQ ID NO: 13; the amino acid I at a residue corresponding to position 162 in SEQ ID NO: 13; the amino acid S at a residue corresponding to position 166 in SEQ ID NO: 13; the amino acid K at a residue corresponding to position 167 in SEQ ID NO: 13; the amino acid T at a residue corresponding to position 168 in SEQ ID NO: 13; the amino acid F at a residue corresponding to position 171 in SEQ ID NO: 13; the amino acid P at a residue corresponding to position 172 in SEQ ID NO: 13; the amino acid F at a residue corresponding to position 175 in SEQ ID NO: 13; the amino acid G at a residue corresponding to position 180 in SEQ ID NO: 13; the amino acid F at a residue corresponding to position 184 in SEQ ID NO: 13; the amino acid K at a residue corresponding to position 196 in SEQ ID NO: 13; the amino acid D at a residue corresponding to position 211 in SEQ ID NO: 13; the amino acid N at a residue corresponding to position 213 in SEQ ID NO: 13, the amino acid L at a residue corresponding to position 216 in SEQ ID NO: 13; the amino acid I at a residue corresponding to position 230 in SEQ ID NO: 13; the amino acid R at a residue corresponding to position 250 in SEQ ID NO: 13; the amino acid L at a residue corresponding to position 263 in SEQ ID NO: 13; the amino acid H at a residue corresponding to position 273 in SEQ ID NO: 13; the amino acid P at a residue corresponding to position 283 in SEQ ID NO: 13; the amino acid T at a residue corresponding to position 287 in SEQ ID NO: 13; the amino acid M or A at a residue corresponding to position 290 in SEQ ID NO: 13; the amino acid M at a residue corresponding to position 292 in SEQ ID NO: 13; the amino acid D or N at a residue corresponding to position 319 in SEQ ID NO: 13; the amino acid E at a residue corresponding to position 322 in SEQ ID NO: 13; the amino acid E at a residue corresponding to position 339 in SEQ ID NO: 13; the amino acid E at a residue corresponding to position 343 in SEQ ID NO: 13; the amino acid M at a residue corresponding to position 344 in SEQ ID NO: 13; the amino acid M at a residue corresponding to position 353 in SEQ ID NO: 13, the amino acid L, Y, A, G, N, P, R, S, T, or V at a residue corresponding to position 376 in SEQ ID NO: 13; the amino acid F, P, or R at a residue corresponding to position 377 in SEQ ID NO: 13; the amino acid K, R, S, or T at a residue corresponding to position 378 in SEQ ID NO: 13; the amino acid Y at a residue corresponding to position 380 in SEQ ID NO: 13; the amino acid F at a residue corresponding to position 386 in SEQ ID NO: 13; the amino acid E at a residue corresponding to position 394 in SEQ ID NO: 13; the amino acid E or K at a residue corresponding to position 397 in SEQ ID NO: 13; the amino acid E at a residue corresponding to position 407 in SEQ ID NO: 13; the amino acid T or V at a residue corresponding to position 410 in SEQ ID NO: 13; the amino acid I, L, M, T, or V at a residue corresponding to position 414 in SEQ ID NO: 13; the amino acid M at a residue corresponding to position 415 in SEQ ID NO: 13; the amino acid F, I, or M at a residue corresponding to position 416 in SEQ ID NO: 13; the amino acid F at a residue corresponding to position 418 in SEQ ID NO: 13; the amino acid S or T at a residue corresponding to position 441 in SEQ ID NO: 13; the amino acid I at a residue corresponding to position 442 in SEQ ID NO: 13; the amino acid V or A at a residue corresponding to position 445 in SEQ ID NO: 13; the amino acid T or V at a residue corresponding to position 446 in SEQ ID NO: 13; the amino acid S at a residue corresponding to position 450 in SEQ ID NO: 13; the amino acid T at a residue corresponding to position 452 in SEQ ID NO: 13; the amino acid A at a residue corresponding to position 454 in SEQ ID NO: 13; the amino acid Y at a residue corresponding to position 467 in SEQ ID NO: 13; the amino acid S or T at a residue corresponding to position 479 in SEQ ID NO: 13; the amino acid I, M, V, or Y at a residue corresponding to position 481 in SEQ ID NO: 13; the amino acid V at a residue corresponding to position 486 in SEQ ID NO: 13; the amino acid T at a residue corresponding to position 490 in SEQ ID NO: 13; the amino acid N at a residue corresponding to position 492 in SEQ ID NO: 13; the amino acid Q at a residue corresponding to position 504 in SEQ ID NO: 13; the amino acid N at a residue corresponding to position 512 in SEQ ID NO: 13; the amino acid D at a residue corresponding to position 527 in SEQ ID NO: 13; and/or the amino acid M at a residue corresponding to position 542 in SEQ ID NO: 13.

In some embodiments, the TS comprises: the amino acid G at a residue corresponding to position 79 in SEQ ID NO: 13; the amino acid C at a residue corresponding to position 90 in SEQ ID NO: 13; the amino acid E at a residue corresponding to position 106 in SEQ ID NO: 13; the amino acid Q at a residue corresponding to position 150 in SEQ ID NO: 13; the amino acid S at a residue corresponding to position 166 in SEQ ID NO: 13; the amino acid D at a residue corresponding to position 211 in SEQ ID NO: 13; the amino acid N at a residue corresponding to position 213 in SEQ ID NO: 13; the amino acid L at a residue corresponding to position 216 in SEQ ID NO: 13; the amino acid I at a residue corresponding to position 230 in SEQ ID NO: 13; the amino acid L at a residue corresponding to position 263 in SEQ ID NO: 13; the amino acid H at a residue corresponding to position 273 in SEQ ID NO: 13; the amino acid P at a residue corresponding to position 283 in SEQ ID NO: 13; the amino acid T at a residue corresponding to position 287 in SEQ ID NO: 13; the amino acid M or A at a residue corresponding to position 290 in SEQ ID NO: 13; the amino acid M at a residue corresponding to position 292 in SEQ ID NO: 13, the amino acid D or N at a residue corresponding to position 319 in SEQ ID NO: 13; the amino acid E at a residue corresponding to position 322 in SEQ ID NO: 13; the amino acid E at a residue corresponding to position 339 in SEQ ID NO: 13; the amino acid E at a residue corresponding to position 343 in SEQ ID NO: 13; the amino acid M at a residue corresponding to position 344 in SEQ ID NO: 13; the amino acid M at a residue corresponding to position 353 in SEQ ID NO: 13; the amino acid Y at a residue corresponding to position 380 in SEQ ID NO: 13; the amino acid F at a residue corresponding to position 386 in SEQ ID NO: 13; the amino acid E at a residue corresponding to position 394 in SEQ ID NO: 13; the amino acid E or K at a residue corresponding to position 397 in SEQ ID NO: 13; the amino acid E at a residue corresponding to position 407 in SEQ ID NO: 13; the amino acid F, I, or M at a residue corresponding to position 416 in SEQ ID NO: 13; the amino acid F at a residue corresponding to position 418 in SEQ ID NO: 13; the amino acid S or T at a residue corresponding to position 441 in SEQ ID NO: 13; the amino acid I at a residue corresponding to position 442 in SEQ ID NO: 13; the amino acid T or V at a residue corresponding to position 446 in SEQ ID NO: 13; the amino acid S at a residue corresponding to position 450 in SEQ ID NO: 13; the amino acid S or T at a residue corresponding to position 479 in SEQ ID NO: 13; the amino acid I, M, V, or Y at a residue corresponding to position 481 in SEQ ID NO: 13; the amino acid V at a residue corresponding to position 486 in SEQ ID NO: 13; the amino acid T at a residue corresponding to position 490 in SEQ ID NO: 13; the amino acid Q at a residue corresponding to position 504 in SEQ ID NO: 13; and/or the amino acid N at a residue corresponding to position 512 in SEQ ID NO: 13.

In some embodiments, the TS comprises at least 2, 3, 4, 5, 6, 7, 8, 9, 10, 11, 12, 13, 14, 15, or 16 amino acid substitutions at residues corresponding to positions 31, 47, 49, 50, 56, 57, 58, 69, 79, 89, 90, 95, 100, 103, 106, 116, 124, 143, 150, 162, 166, 167, 168, 171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416,418, 441, 442, 445,446, 479, 450, 452, 454, 467, 481, 486, 490, 492, 504, 512, 527 and/or 542 in SEQ ID NO: 13. In some embodiments, the TS comprises at least 2, 3, 4, 5, 6, 7, 8, 9, 10, 11, 12, 13, 14, 15, or 16 amino acid substitutions at residues corresponding to positions 79, 90, 106, 150, 166, 184, 211, 216, 230, 263, 273, 283, 290, 292, 319, 322, 339, 353, 380, 386, 397, 407, 416,418, 441, 442, 446, 479, 450, 452, 454, 467, 481, 486, 504, and/or 512 in SEQ ID NO: 13.

In some embodiments, the TS comprises relative to SEQ ID NO: 13: K50N, G95A, N196K, H213N, T339E, Q343E, L344M, and A414V; G95A, Y175F, T339E, Q343E, and A414V; G95A, S116A, T339E, Q343E, A414V, and N527D; G95A, E150Q, V162L, C180G, N196K, N211D, N273H, T339E, Q343E, and A414V; G95A, T339E, Q343E, Q376V, and A414V; K50N, G95A, S100A, E150Q, V162I, C180G, N196K, N211 D, H213N, S322E, T339E, Q343E, L344M, A414V, E452T, and I504Q; G95A, N196K, T339E, Q343E, and A414V; 50N, G95A, V103H, H213N, T339E, Q343E, L344M, and A414V; G95A, T339E, Q343E, Q376R, and A414V; or K50N, H213N, L230I, T339E, Q343E, and L344M.

In some embodiments, the TS comprises relative to SEQ ID NO: 13: K50N, H213N, L230I, T339E, Q343E, and L344M; S100A, T339E, and Q343E; T339E, Q343E, L344M, and N527D; K50N, V162I, C180G, N196K, N211D, H213N, T339E, Q343E, and L344M; K50N, E150Q, V162I, C180G, N196K, N211 D, H213N, T339E, Q343E, and L344M; S116A, H213N, T339E, Q343E, L344M, and N527D; N196K, T339E, and Q343E; K50N, E150Q, V1621, A172P, C180G, N196K, N211D, H213N, T339E, Q343E, and L344M; V216L, T339E, and Q343E; S116A, H213N, T339E, Q343E, and N527D; S116A, T339E, Q343E, and N527D; or T339E, Q343E, and Q376P.

In some embodiments, the TS comprises a sequence that is at least 90%, at least 95%, at least 96%, at least 97%, at least 98%, or at least 99% identical to any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948, or a conservatively substituted version thereof. In some embodiments, the TS comprises a sequence that is at least 90%, at least 95%, at least 96%, at least 97%, at least 98%, or at least 99% identical to any one of SEQ ID NOs: 784, 786, 792, 804, 828, 801, 806, 830, 808, 813, 809, 800, 815, 816 836, 825, 791, 845, 823, and 820, or a conservatively substituted version thereof. In some embodiments, the TS comprises a sequence that is at least 90%, at least 95%, at least 96%, at least 97%, at least 98%, or at least 99% identical to any one of SEQ ID NOs: 795, 812, 816, 817, 823, 825, 853, 868, 874, 946, 948, and 949, or a conservatively substituted version thereof. In some embodiments, the TS comprises the sequence of any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948, or a conservatively substituted version thereof.

Further aspects of the disclosure relate to host cells that comprise a heterologous polynucleotide encoding a TS, wherein the TS comprises a sequence that is at least 98% identical to SEQ ID NO: 36, and wherein the host cell is capable of producing a CBD-type cannabinoid. In some embodiments, the CBD-type cannabinoid is CBDA and/or CBDVA. In some embodiments, the TS is capable of producing more of a CBD-type cannabinoid than a control TS, wherein the control TS comprises the sequence of SEQ ID NO: 136. In some embodiments, the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBD-type cannabinoid than a control TS, wherein the control TS comprises the sequence of SEQ ID NO: 136.

In some embodiments, the TS further comprises a first signal peptide. In some embodiments, the first signal peptide comprises SEQ ID NO: 16 or a sequence that has no more than two amino acid substitutions, insertions, additions, or deletions relative to the sequence of SEQ ID NO: 16. In some embodiments, the first signal peptide is located at the amino terminus of the TS. In some embodiments, a methionine residue is added to the N-terminus of SEQ ID NO: 16. In some embodiments, the TS further comprises a second signal peptide. In some embodiments, the second signal peptide comprises SEQ ID NO: 17 or a sequence that has no more than one amino acid substitution, insertion, addition, or deletion relative to the sequence of SEQ ID NO: 17. In some embodiments, the second signal peptide is located at the carboxyl terminus of the TS.

In some embodiments, the host cell further produces one or more of THCA, THCVA, CBCA and/or CBCVA. In some embodiments, the TS produces a higher ratio of CBDA:THCA, CBDA:CBCA, CBDVA:THCVA and/or CBCVA:THCVA than a control TS. In some embodiments, the control TS is a TS comprising the sequence of SEQ ID NO: 136. In some embodiments, the TS has a higher product specificity for a CBD-type cannabinoid than a control TS. In some embodiments, the control TS is a TS comprising the sequence of SEQ ID NO: 136.

Further aspects of the disclosure relate to host cells that comprise a heterologous polynucleotide encoding a TS, wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14, and wherein the TS is capable of producing a CBC-type cannabinoid.

In some embodiments, relative to the sequence of SEQ ID NO: 14, the TS further comprises an amino acid substitution at one or more residues corresponding to positions 31, 40, 46, 74, 90, 255, 288, 290, 318, and/or 495 in SEQ ID NO: 14. In some embodiments, the TS is capable of producing more of a CBC-type cannabinoid than a control TS, and wherein the control TS comprises the sequence of SEQ ID NO: 21. In some embodiments, a control TS, or a polynucleotide encoding a control TS, comprises the sequence of any one of SEQ ID NOs: 20, 21, 22, 23, or 24.

Further aspects of the disclosure relate to host cells that comprise a heterologous polynucleotide encoding a TS, wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14, wherein the TS is capable of producing more of a CBC-type cannabinoid than a control TS, and wherein the control TS comprises the sequence of SEQ ID NO: 21. In some embodiments, a control TS, or a polynucleotide encoding a control TS, comprises the sequence of any one of SEQ ID NOs: 20, 21, 22, 23, or 24.

In some embodiments, the CBC-type cannabinoid is CBCA and/or CBCVA. In some embodiments, the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBC-type cannabinoid than a control TS, wherein the control TS comprises the sequence of SEQ ID NO: 21. In some embodiments, the TS is capable of producing at least 1, 2, 3, or 4-fold more of a CBC-type cannabinoid than a control TS, wherein the control TS comprises the sequence of SEQ ID NO: 21. In some embodiments, a control TS, or a polynucleotide encoding a control TS, comprises the sequence of any one of SEQ ID NOs: 20, 21, 22, 23, or 24.

In some embodiments, the TS comprises: the amino acid Q at a residue corresponding to position 31 in SEQ ID NO: 14; the amino acid E at a residue corresponding to position 40 in SEQ ID NO: 14; the amino acid Y at a residue corresponding to position 41 in SEQ ID NO: 14; the amino acid P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid S at a residue corresponding to position 58 in SEQ ID NO: 14; the amino acid S at a residue corresponding to position 61 in SEQ ID NO: 14; the amino acid V or L at a residue corresponding to position 63 in SEQ ID NO: 14; the amino acid T at a residue corresponding to position 74 in SEQ ID NO: 14; the amino acid V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid V at a residue corresponding to position 255 in SEQ ID NO: 14; the amino acid M at a residue corresponding to position 257 in SEQ ID NO: 14; the amino acid E at a residue corresponding to position 268 in SEQ ID NO: 14; the amino acid V at a residue corresponding to position 273 in SEQ ID NO: 14; the amino acid L at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid E at a residue corresponding to position 340 in SEQ ID NO: 14; the amino acid Q at a residue corresponding to position 344 in SEQ ID NO: 14; the amino acid L at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid S at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I at a residue corresponding to position 442 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14: the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid I at a residue corresponding to position 489 in SEQ ID NO: 14; the amino acid I at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid F or P at a residue corresponding to position 494 in SEQ ID NO: 14; the amino acid E or K at a residue corresponding to position 495 in SEQ ID NO: 14; the amino acid N at a residue corresponding to position 496 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the TS comprises: the amino acid Y at a residue corresponding to position 41 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid S at a residue corresponding to position 58 in SEQ ID NO: 14; the amino acid S at a residue corresponding to position 61 in SEQ ID NO: 14; the amino acid V or L at a residue corresponding to position 63 in SEQ ID NO: 14; the amino acid G at a residue corresponding to position 95 in SEQ ID NO: 14; the amino acid S at a residue corresponding to position 96 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid M at a residue corresponding to position 257 in SEQ ID NO: 14; the amino acid E at a residue corresponding to position 268 in SEQ ID NO: 14; the amino acid V at a residue corresponding to position 273 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid E at a residue corresponding to position 340 in SEQ ID NO: 14; the amino acid Q at a residue corresponding to position 344 in SEQ ID NO: 14; the amino acid L at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid S at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I at a residue corresponding to position 442 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid I at a residue corresponding to position 489 in SEQ ID NO: 14, the amino acid I at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid F or P at a residue corresponding to position 494 in SEQ ID NO: 14; the amino acid N at a residue corresponding to position 496 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the TS comprises at least 2, 3, 4, 5, 6, 7, 8, 9, 10, 11, 12, 13, 14, 15, or 16 amino acid substitutions at residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14. In some embodiments, the TS comprises at least 2, 3, 4, 5, 6, 7, 8, 9, 10, 11, 12, 13, 14, 15, or 16 amino acid substitutions at residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14.

In some embodiments, the TS comprises relative to SEQ ID NO: 14: Q58S, V288L, and F345L; R31Q, V52I, H56N, Q58S, M61S, I74T, N90V, A250P, S255V, F345L, Q475K, and T492N; R31Q, H56N, 174T, N90V, H143E, A250P, S255V, Q475K, and T492N; R31Q, H56N, I74T, N90V, A250P, S255V, L443I, Q475K, and T492N; H56N, M61S, N90V, A250D, S255V, V288L, Q475K, T492N, and A495E; R31Q, H56N, 174T, N90V, K215R, A250P, S255V, Q475K, and T492N; R31Q, P49A, H56N, Q58S, M61S, I74T, N90V, H143E, A250P, S255V, V288L, F345L, Q475K, and T492N; R31Q, A47T, H56N, I74T, N90V, A250P, S255V, Q475K, and T492N; M61S, N90V, A250D, S255V, Q475K, T492N, A495E, and N498T; R31Q, H56N, M61S, I74T, N89H, N90V, S100A, H136R, E150Q, N196K, N211D, A250P, S255V, V288M, F345M, S382K, L443I, Q475K, and T492N; R31Q, H56N, Q58S, M61S, I74T, N90V, H143E, A250P, S255V, V288L, T340E, F345L, E424D, Q475K, and T492N; R31Q, H56N, I74T, S88L, N90V, A250P, S255V, Q475K, and T492N; R31Q, V52I, H56N, Q58S, M61S, 174T, N90V, H143E, A250P, S255V, V288L, F345L, Q475K, and T492N; R31Q, H56N, Q58S, M61S, 174T, N90V, H143E, A250P, S255V, V288L, T340E, F345L, E424D, Q475K, and T492N; R31Q, H56N, Q58S, M61S, I74T, N90V, H143E, A250P, S255V, V288L, F345L, A411V, Q475K, and T492N; R31Q, V52I, H56N, Q58S, M61S, I74T, N90V, A250P, S255V, V288L, F345L, Q475K, and T492N; R31Q, K50L, H56N, I74T, N90V, A250P, S255V, Q475K, and T492N; R31Q, A47T, V52I, H56N, Q58S, M61S, I74T, N90V, H143E, A250P, S255V, V288L, T340E, F345L, Q475K, and T492N; or R31Q, H56N, M61S, I74T, N89H, N90V, S100A, N196K, N211D, A250P, S255V, I257R, V288M, F345M, S382K, L443I, Q475K, and T492N.

In some embodiments, the TS comprises a sequence that is at least 90%, at least 95%, at least 97%, at least 98%, or at least 99% identical to any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993, or a conservatively substituted version thereof. In some embodiments, the TS comprises a sequence that is at least 90%, at least 95%, at least 97%, at least 98%, or at least 99% identical to any one of SEQ ID NOs: 698-716, or a conservatively substituted version thereof. In some embodiments, the TS comprises the sequence of any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993, or a conservatively substituted version thereof.

Further aspects of the disclosure relate to host cells that comprise a heterologous polynucleotide encoding a TS, wherein the TS comprises a sequence that is at least 98% identical to SEQ ID NO: 39, and wherein the host cell is capable of producing a CBC-type cannabinoid. In some embodiments, the CBC-type cannabinoid is CBCA and/or CBCVA. In some embodiments, the TS is capable of producing more of a CBC-type cannabinoid than a control TS, wherein the control TS comprises the sequence of SEQ ID NO: 21. In some embodiments, a control TS, or a polynucleotide encoding a control TS, comprises the sequence of any one of SEQ ID NOs: 20, 21, 22, 23, or 24. In some embodiments, the TS is capable of producing at least 0.05%, 0.075%, 0.1%, 0.5%, 0.75%, 1%, 5%, 10%, 20%, 30%, 40%, 50%4, 60%, 70%, 80%, 90%, 120%, 150%, 170%, 200%, 240%, 290%, or 300% more of a CBC-type cannabinoid than a control TS, wherein the control TS comprises the sequence SEQ ID NO: 21. In some embodiments, a control TS, or a polynucleotide encoding a control TS, comprises the sequence of any one of SEQ ID NOs: 20, 21, 22, 23, or 24.

In some embodiments, the TS further comprises a first signal peptide. In some embodiments, the first signal peptide comprises SEQ ID NO: 16 or a sequence that has no more than two amino acid substitutions, insertions, additions, or deletions relative to the sequence of SEQ ID NO: 16. In some embodiments, the first signal peptide is located at the amino terminus of the TS. In some embodiments, a methionine residue is added to the N-terminus of SEQ ID NO: 16. In some embodiments, the TS further comprises a second signal peptide. In some embodiments, the second signal peptide comprises SEQ ID NO: 17 or a sequence that has no more than one amino acid substitution, insertion, addition, or deletion relative to the sequence of SEQ ID NO: 17. In some embodiments, the second signal peptide is located at the carboxyl terminus of the TS.

In some embodiments, the host cell further produces one or more of THCA, THCVA, CBDA and/or CBDVA. In some embodiments, the TS produces a higher ratio of CBCA:THCA, CBCA:CBDA, CBCVA:THCVA, and/or CBCVA: CBDVA than a control TS. In some embodiments, the control TS is a TS comprising the sequence of SEQ ID NO: 21. In some embodiments, the TS has a higher product specificity for a THC-type cannabinoid than a control TS. In some embodiments, the control TS is a TS comprising the sequence of SEQ ID NO: 21. In some embodiments, a control TS, or a polynucleotide encoding a control TS, comprises the sequence of any one of SEQ ID NOs: 20, 21, 22, 23, or 24.

In some embodiments, the host cell is a plant cell, an algal cell, a yeast cell, a bacterial cell, or an animal cell. In some embodiments, the host cell is a yeast cell. In some embodiments, the yeast cell is a Saccharomyces cell, a Yarrowia cell, a Komagataella cell, or a Pichia cell. In some embodiments, the Saccharomyces cell is a Saccharomyces cerevisiae cell. In some embodiments, the yeast cell is a Yarrowia cell. In some embodiments, the host cell is a bacterial cell. In some embodiments, the bacterial cell is an E. coli cell. In some embodiments, the host cell further comprises one or more heterologous polynucleotides encoding one or more of: an acyl activating enzyme (AAE), a polyketide synthase (PKS), a polyketide cyclase (PKC), a prenyltransferase (PT), and/or an additional terminal synthase (TS). In some embodiments, the PKS is an olivetol synthase (OLS) or a divarinol synthase.

Further aspects of the disclosure relate to methods comprising culturing any of the host cells associated with the disclosure.

Further aspects of the disclosure relate to methods for producing a cannabinoid comprising contacting a CBG-type cannabinoid with a TS, wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14.

Further aspects of the disclosure relate to methods for producing a cannabinoid comprising contacting a CBG-type cannabinoid with a TS, wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14, wherein the TS does not comprise SEQ ID NO: 20, 21, 320 or 321, wherein the TS is capable of producing more of a THC-type cannabinoid than a control TS, and wherein the control TS comprises the sequence of SEQ ID NO: 284 or SEQ ID NO: 21. In some embodiments, a control TS, or a polynucleotide encoding a control TS, comprises the sequence of any one of SEQ ID NOs: 20, 21, 22, 23, 24, 14, 284, 254, or 1220.

Further aspects of the disclosure relate to methods for producing a cannabinoid comprising contacting a CBG-type cannabinoid with a TS, wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13.

Further aspects of the disclosure relate to methods for producing a cannabinoid comprising contacting a CBG-type cannabinoid with a TS, wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168,171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13, wherein the TS is capable of producing more of a CBD-type cannabinoid than a control TS, and wherein the control TS comprises the sequence of SEQ ID NO: 136.

Further aspects of the disclosure relate to methods for producing a cannabinoid comprising contacting a CBG-type cannabinoid with a TS, wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14 and wherein the TS is capable of producing a CBC-type cannabinoid.

Further aspects of the disclosure relate to methods for producing a cannabinoid comprising contacting a CBG-type cannabinoid with a TS, wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14, wherein the TS is capable of producing more of a CBC-type cannabinoid than a control TS, and wherein the control TS comprises the sequence of SEQ ID NO: 21. In some embodiments, a control TS, or a polynucleotide encoding a control TS, comprises the sequence of any one of SEQ ID NOs: 20, 21, 22, 23, or 24.

In some embodiments, contacting the CBG-type cannabinoid with the TS occurs in vitro. In some embodiments, contacting the CBG-type cannabinoid with the TS occurs in vivo. In some embodiments, contacting the CBG-type cannabinoid with the TS occurs in a host cell.

Further aspects of the disclosure relate to non-naturally occurring TSs, wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419,424, 443, 459, 462, 464,469, 479, 475, 491, 492, and/or 499 in SEQ ID NO: 14, and wherein the TS is capable of producing a THC-type cannabinoid.

In some embodiments, relative to the sequence of SEQ ID NO: 14, the TS further comprises an amino acid substitution at one or more residues corresponding to positions 31, 40, 41, 46, 49, 51, 56, 59, 61, 63, 74, 90, 96, 100, 103, 116, 143, 173, 196, 250, 257, 290, 296, 311, 354, 377, 378, 411, 417, 446, 494, 495, 528, 542, 543 and/or 544 in SEQ ID NO: 14, wherein the TS does not comprise the sequence of SEQ ID NO: 20, 21, 320 or 321.

In some embodiments, the TS comprises: the amino acid Q at a residue corresponding to position 31 in SEQ ID NO: 14; the amino acid H or Q at a residue corresponding to position 36 in SEQ ID NO: 14; the amino acid E or Q at a residue corresponding to position 40 in SEQ ID NO: 14; the amino acid Y at a residue corresponding to position 41 in SEQ ID NO: 14; the amino acid T at a residue corresponding to position 44 in SEQ ID NO: 14, the amino acid A or P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14, the amino acid P or S at a residue corresponding to position 58 in SEQ ID NO: 14; the amino acid F at a residue corresponding to position 59 in SEQ ID NO: 14; the amino acid S at a residue corresponding to position 61 in SEQ ID NO: 14; the amino acid L or V at a residue corresponding to position 63 in SEQ ID NO: 14; the amino acid T at a residue corresponding to position 74 in SEQ ID NO: 14; the amino acid N at a residue corresponding to position 76 in SEQ ID NO: 14; the amino acid I at a residue corresponding to position 85 in SEQ ID NO: 14; the amino acid L at a residue corresponding to position 88 in SEQ ID NO: 14; the amino acid D, E, or H at a residue corresponding to position 89 in SEQ ID NO: 14; the amino acid E or V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid A at a residue corresponding to position 100 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid Q at a residue corresponding to position 150 in SEQ ID NO: 14; the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid K at a residue corresponding to position 196 in SEQ ID NO: 14; the amino acid D at a residue corresponding to position 211 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid D or P at a residue corresponding to position 250 in SEQ ID NO: 14; the amino acid V at a residue corresponding to position 255 in SEQ ID NO: 14; the amino acid M or R at a residue corresponding to position 257 in SEQ ID NO: 14; the amino acid N at a residue corresponding to position 267 in SEQ ID NO: 14; the amino acid E at a residue corresponding to position 268 in SEQ ID NO: 14; the amino acid V at a residue corresponding to position 273 in SEQ ID NO: 14; the amino acid H at a residue corresponding to position 274 in SEQ ID NO: 14; the amino acid L, M, or T at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid Q at a residue corresponding to position 329 in SEQ ID NO: 14; the amino acid E at a residue corresponding to position 340 in SEQ ID NO: 14; the amino acid Q at a residue corresponding to position 344 in SEQ ID NO: 14; the amino acid L or M at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid K at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid V at a residue corresponding to position 417 in SEQ ID NO: 14; the amino acid F at a residue corresponding to position 419 in SEQ ID NO: 14; the amino acid D at a residue corresponding to position 424 in SEQ ID NO: 14; the amino acid I at a residue corresponding to position 443 in SEQ ID NO: 14; the amino acid I at a residue corresponding to position 446 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid M at a residue corresponding to position 491 in SEQ ID NO: 14; the amino acid N at a residue corresponding to position 492 in SEQ ID NO: 14; the amino acid D, E, F, or P at a residue corresponding to position 494 in SEQ ID NO: 14; the amino acid E or K at a residue corresponding to position 495 in SEQ ID NO: 14; the amino acid Q at a residue corresponding to position 499 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the TS comprises: the amino acid H or Q at a residue corresponding to position 36 in SEQ ID NO: 14; the amino acid T at a residue corresponding to position 44 in SEQ ID NO: 14; the amino acid T at a residue corresponding to position 47 in SEQ ID NO: 14; the amino acid I at a residue corresponding to position 52 in SEQ ID NO: 14; the amino acid P or S at a residue corresponding to position 58 in SEQ ID NO: 14; the amino acid I at a residue corresponding to position 85 in SEQ ID NO: 14; the amino acid L at a residue corresponding to position 88 in SEQ ID NO: 14; the amino acid D, E, or H at a residue corresponding to position 89 in SEQ ID NO: 14; the amino acid G at a residue corresponding to position 95 in SEQ ID NO: 14; the amino acid I at a residue corresponding to position 129 in SEQ ID NO: 14; the amino acid R at a residue corresponding to position 136 in SEQ ID NO: 14; the amino acid Q at a residue corresponding to position 150 in SEQ ID NO: 14; the amino acid L at a residue corresponding to position 158 in SEQ ID NO: 14; the amino acid A at a residue corresponding to position 181 in SEQ ID NO: 14; the amino acid D at a residue corresponding to position 211 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid V at a residue corresponding to position 255 in SEQ ID NO: 14; the amino acid N at a residue corresponding to position 267 in SEQ ID NO: 14; the amino acid E at a residue corresponding to position 268 in SEQ ID NO: 14; the amino acid V at a residue corresponding to position 273 in SEQ ID NO: 14; the amino acid H at a residue corresponding to position 274 in SEQ ID NO: 14; the amino acid L, M, or T at a residue corresponding to position 288 in SEQ ID NO: 14; the amino acid Q at a residue corresponding to position 302 in SEQ ID NO: 14; the amino acid I at a residue corresponding to position 309 in SEQ ID NO: 14; the amino acid L at a residue corresponding to position 318 in SEQ ID NO: 14, the amino acid Q at a residue corresponding to position 329 in SEQ ID NO: 14; the amino acid E at a residue corresponding to position 340 in SEQ ID NO. 14; the amino acid Q at a residue corresponding to position 344 in SEQ ID NO: 14; the amino acid L or M at a residue corresponding to position 345 in SEQ ID NO: 14; the amino acid I at a residue corresponding to position 351 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid A at a residue corresponding to position 379 in SEQ ID NO: 14; the amino acid K at a residue corresponding to position 382 in SEQ ID NO: 14; the amino acid V at a residue corresponding to position 396 in SEQ ID NO: 14; the amino acid F at a residue corresponding to position 419 in SEQ ID NO: 14; the amino acid D at a residue corresponding to position 424 in SEQ ID NO: 14; the amino acid I at a residue corresponding to position 443 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid M at a residue corresponding to position 469 in SEQ ID NO: 14; the amino acid M at a residue corresponding to position 479 in SEQ ID NO: 14; the amino acid M at a residue corresponding to position 491 in SEQ ID NO: 14; the amino acid N at a residue corresponding to position 492 in SEQ ID NO: 14; and/or the amino acid Q at a residue corresponding to position 499 in SEQ ID NO: 14.

In some embodiments, the TS comprises relative to SEQ ID NO: 14: R31Q, H56N, Q58S, M61 S, 174T, N90V, H143E, A250P, S255V, V288L, T340E, F345L, E424D, Q475K, T492N, and P542L; R31Q, V52I, H56N, Q58S, M61 S, I74T, N90V, A250P, S255V, V288L, F345L, Q475K, and T492N; R31Q, A47T, V52I, H56N, Q58S, M61S, I74T, N90V, H143E, A250P, S255V, V288L, T340E, F345L, Q475K, and T492N; H56N, Q58S, M61S, I74T, N90V, H143E, A250D, S255V, V288L, F345L, Q475K, T492N, and A495E; R31Q, H56N, Q58S, M61S, I74T, N90V, H143E, A250P, S255V, V288L, Q475K, and T492N; R31Q, A47T, H56N, Q58S, M61S, I74T, N90V, H143E, A250P, S255V, V288L, T340E, F345L, E424D, Q475K, and T492N; A47T, H56N, Q58S, M61S, I74T, N90V, A250D, S255V, F345L, E424D, Q475K, and T492N; R31Q, A47T, H56N, Q58S, M61S, 174T, N90V, A250P, S255V, V288L, T340E, F345L, Q475K, and T492N; R31Q, A47T, H56N, Q58S, M61S, 174T, N90V, H143E, A250P, S255V, V288L, T340E, F345L, Q475K, and T492N; A47T, H56N, Q58S, M61S, 174T, N90V, A250D, S255V, F345L, Q475K, and T492N; R31Q, A47T, H56N, Q58S, M61 S, 174T, N90V, H143E, A250P, S255V, T340E, F345L, Q475K, and T492N; R31Q, H56N, Q58S, M61S, I74T, N90V, A250P, S255V, V288L, F345L, E424D, Q475K, and T492N; R31Q, A47T, H56N, Q58S, M61S, I74T, N90V, A250P, S255V, T340E, F345L, E424D, Q475K, and T492N; A47T, H56N, Q58S, M61S, 174T, N90V, A250D, S255V, V288L, F345L, Q475K, and T492N; H56N, Q58S, M61S, I74T, N90V, H143E, A250D, S255V, V288L, F345L, Q475K, and T492N; or R31Q, V52I, H56N, M61S, I74T, N90V, A250P, S255V, F345L, Q475K, and T492N.

In some embodiments, the TS comprises relative to SEQ ID NO: 14: M61S, N90V, A250D, S255V, Q475K, T492N, and A495E; H56N, M61S, 174T, N90V, A250P, S255V, T492N, and H494E; or R31Q, H56N, I74T, N90V, A250P, S255V, Q475K, T492N, H494E, and A495E.

In some embodiments, the TS comprises a sequence that is at least 90%, at least 95%, at least 97%, at least 98%, at least 99% identical, or is 100% identical to any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913, or a conservatively substituted version thereof.

Further aspects of the disclosure relate to non-naturally occurring TSs, wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479,450, 452, 454, 467, 481, 486, 504, and/or 512 in SEQ ID NO: 13, and wherein the TS is capable of producing a CBD-type cannabinoid.

In some embodiments, relative to the sequence of SEQ ID NO: 13, the TS further comprises an amino acid substitution at one or more residues corresponding to positions 31, 47, 49, 50, 56, 57, 58, 69, 89, 95, 100, 103, 116, 124, 143, 162, 167, 168, 171, 172, 175, 180, 196, 213, 250, 287, 343, 344, 376, 377, 378, 394, 410, 414, 415, 445, 490, 492, 517 and/or 542 in SEQ ID NO: 13.

In some embodiments, the TS comprises: the amino acid Q at a residue corresponding to position 31 in SEQ ID NO: 13; the amino acid A at a residue corresponding to position 47 in SEQ ID NO: 13; the amino acid P at a residue corresponding to position 49 in SEQ ID NO: 13; the amino acid N at a residue corresponding to position 50 in SEQ ID NO: 13; the amino acid H at a residue corresponding to position 56 in SEQ ID NO: 13; the amino acid D at a residue corresponding to position 57 in SEQ ID NO: 13; the amino acid Q at a residue corresponding to position 58 in SEQ ID NO: 13; the amino acid R or Q at a residue corresponding to position 69 in SEQ ID NO: 13; the amino acid G at a residue corresponding to position 79 in SEQ ID NO: 13; the amino acid N, D, E, Q, or R at a residue corresponding to position 89 in SEQ ID NO: 13; the amino acid C at a residue corresponding to position 90 in SEQ ID NO: 13; the amino acid A at a residue corresponding to position 95 in SEQ ID NO: 13; the amino acid A at a residue corresponding to position 100 in SEQ ID NO: 13; the amino acid H at a residue corresponding to position 103 in SEQ ID NO: 13; the amino acid E at a residue corresponding to position 106 in SEQ ID NO: 13, the amino acid A or G at a residue corresponding to position 116 in SEQ ID NO: 13, the amino acid N or M at a residue corresponding to position 124 in SEQ ID NO: 13; the amino acid E at a residue corresponding to position 143 in SEQ ID NO: 13; the amino acid Q at a residue corresponding to position 150 in SEQ ID NO: 13; the amino acid I at a residue corresponding to position 162 in SEQ ID NO: 13; the amino acid S at a residue corresponding to position 166 in SEQ ID NO: 13; the amino acid K at a residue corresponding to position 167 in SEQ ID NO: 13; the amino acid T at a residue corresponding to position 168 in SEQ ID NO: 13; the amino acid F at a residue corresponding to position 171 in SEQ ID NO: 13; the amino acid P at a residue corresponding to position 172 in SEQ ID NO: 13; the amino acid F at a residue corresponding to position 175 in SEQ ID NO: 13; the amino acid G at a residue corresponding to position 180 in SEQ ID NO: 13; the amino acid F at a residue corresponding to position 184 in SEQ ID NO: 13; the amino acid K at a residue corresponding to position 196 in SEQ ID NO: 13; the amino acid D at a residue corresponding to position 211 in SEQ ID NO: 13; the amino acid N at a residue corresponding to position 213 in SEQ ID NO: 13; the amino acid L at a residue corresponding to position 216 in SEQ ID NO: 13; the amino acid I at a residue corresponding to position 230 in SEQ ID NO: 13; the amino acid R at a residue corresponding to position 250 in SEQ ID NO: 13; the amino acid L at a residue corresponding to position 263 in SEQ ID NO: 13; the amino acid H at a residue corresponding to position 273 in SEQ ID NO: 13; the amino acid P at a residue corresponding to position 283 in SEQ ID NO: 13; the amino acid T at a residue corresponding to position 287 in SEQ ID NO: 13; the amino acid M or A at a residue corresponding to position 290 in SEQ ID NO: 13; the amino acid M at a residue corresponding to position 292 in SEQ ID NO: 13; the amino acid D or N at a residue corresponding to position 319 in SEQ ID NO: 13; the amino acid E at a residue corresponding to position 322 in SEQ ID NO: 13; the amino acid E at a residue corresponding to position 339 in SEQ ID NO: 13; the amino acid E at a residue corresponding to position 343 in SEQ ID NO: 13; the amino acid M at a residue corresponding to position 344 in SEQ ID NO: 13; the amino acid M at a residue corresponding to position 353 in SEQ ID NO: 13; the amino acid L, Y, A, G, N, P, R, S, T, or V at a residue corresponding to position 376 in SEQ ID NO: 13; the amino acid F, P, or R at a residue corresponding to position 377 in SEQ ID NO: 13; the amino acid K, R, S, or T at a residue corresponding to position 378 in SEQ ID NO: 13; the amino acid Y at a residue corresponding to position 380 in SEQ ID NO: 13; the amino acid F at a residue corresponding to position 386 in SEQ ID NO: 13; the amino acid E at a residue corresponding to position 394 in SEQ ID NO: 13; the amino acid E or K at a residue corresponding to position 397 in SEQ ID NO: 13; the amino acid E at a residue corresponding to position 407 in SEQ ID NO: 13; the amino acid T or V at a residue corresponding to position 410 in SEQ ID NO: 13; the amino acid I, L, M, T, or V at a residue corresponding to position 414 in SEQ ID NO: 13; the amino acid M at a residue corresponding to position 415 in SEQ ID NO: 13; the amino acid F, I, or M at a residue corresponding to position 416 in SEQ ID NO: 13; the amino acid F at a residue corresponding to position 418 in SEQ ID NO: 13; the amino acid S or T at a residue corresponding to position 441 in SEQ ID NO: 13; the amino acid I at a residue corresponding to position 442 in SEQ ID NO: 13; the amino acid V or A at a residue corresponding to position 445 in SEQ ID NO: 13; the amino acid T or V at a residue corresponding to position 446 in SEQ ID NO: 13; the amino acid S at a residue corresponding to position 450 in SEQ ID NO: 13; the amino acid T at a residue corresponding to position 452 in SEQ ID NO: 13; the amino acid A at a residue corresponding to position 454 in SEQ ID NO: 13; the amino acid Y at a residue corresponding to position 467 in SEQ ID NO: 13; the amino acid S or T at a residue corresponding to position 479 in SEQ ID NO: 13; the amino acid I, M, V, or Y at a residue corresponding to position 481 in SEQ ID NO: 13; the amino acid V at a residue corresponding to position 486 in SEQ ID NO: 13; the amino acid T at a residue corresponding to position 490 in SEQ ID NO: 13; the amino acid N at a residue corresponding to position 492 in SEQ ID NO. 13; the amino acid Q at a residue corresponding to position 504 in SEQ ID NO: 13; the amino acid N at a residue corresponding to position 512 in SEQ ID NO: 13; the amino acid D at a residue corresponding to position 527 in SEQ ID NO: 13, and/or the amino acid M at a residue corresponding to position 542 in SEQ ID NO: 13.

In some embodiments, the TS comprises: the amino acid G at a residue corresponding to position 79 in SEQ ID NO: 13; the amino acid C at a residue corresponding to position 90 in SEQ ID NO: 13; the amino acid E at a residue corresponding to position 106 in SEQ ID NO: 13; the amino acid Q at a residue corresponding to position 150 in SEQ ID NO: 13; the amino acid S at a residue corresponding to position 166 in SEQ ID NO: 13; the amino acid D at a residue corresponding to position 211 in SEQ ID NO: 13; the amino acid N at a residue corresponding to position 213 in SEQ ID NO: 13; the amino acid L at a residue corresponding to position 216 in SEQ ID NO: 13; the amino acid I at a residue corresponding to position 230 in SEQ ID NO: 13; the amino acid L at a residue corresponding to position 263 in SEQ ID NO: 13; the amino acid H at a residue corresponding to position 273 in SEQ ID NO: 13; the amino acid P at a residue corresponding to position 283 in SEQ ID NO: 13; the amino acid T at a residue corresponding to position 287 in SEQ ID NO: 13; the amino acid M or A at a residue corresponding to position 290 in SEQ ID NO: 13; the amino acid M at a residue corresponding to position 292 in SEQ ID NO: 13; the amino acid D or N at a residue corresponding to position 319 in SEQ ID NO: 13; the amino acid E at a residue corresponding to position 322 in SEQ ID NO: 13; the amino acid E at a residue corresponding to position 339 in SEQ ID NO: 13; the amino acid E at a residue corresponding to position 343 in SEQ ID NO: 13; the amino acid M at a residue corresponding to position 344 in SEQ ID NO: 13; the amino acid M at a residue corresponding to position 353 in SEQ ID NO: 13; the amino acid Y at a residue corresponding to position 380 in SEQ ID NO: 13; the amino acid F at a residue corresponding to position 386 in SEQ ID NO: 13; the amino acid E at a residue corresponding to position 394 in SEQ ID NO: 13, the amino acid E or K at a residue corresponding to position 397 in SEQ ID NO: 13; the amino acid E at a residue corresponding to position 407 in SEQ ID NO: 13; the amino acid F, I, or M at a residue corresponding to position 416 in SEQ ID NO: 13; the amino acid F at a residue corresponding to position 418 in SEQ ID NO: 13; the amino acid S or T at a residue corresponding to position 441 in SEQ ID NO: 13; the amino acid I at a residue corresponding to position 442 in SEQ ID NO: 13; the amino acid T or V at a residue corresponding to position 446 in SEQ ID NO: 13; the amino acid S at a residue corresponding to position 450 in SEQ ID NO: 13; the amino acid S or T at a residue corresponding to position 479 in SEQ ID NO: 13; the amino acid I, M, V, or Y at a residue corresponding to position 481 in SEQ ID NO: 13; the amino acid V at a residue corresponding to position 486 in SEQ ID NO: 13; the amino acid T at a residue corresponding to position 490 in SEQ ID NO: 13; the amino acid Q at a residue corresponding to position 504 in SEQ ID NO: 13; and/or the amino acid N at a residue corresponding to position 512 in SEQ ID NO: 13.

In some embodiments, the TS comprises relative to SEQ ID NO. 13: K50N, G95A, N196K, H213N, T339E, Q343E, L344M, and A414V; G95A, Y175F, T339E, Q343E, and A414V; G95A, S116A, T339E, Q343E, A414V, and N527D; G95A, E150Q, V1621, C180G, N196K, N211D, N273H, T339E, Q343E, and A414V; G95A, T339E, Q343E, Q376V, and A414V; K50N, G95A, S100A, E150Q, V1621, C180G, N196K, N211 D, H213N, S322E, T339E, Q343E, L344M, A414V, E452T, and 1504Q; G95A, N196K, T339E, Q343E, and A414V; 50N, G95A, V103H, H213N, T339E, Q343E, L344M, and A414V; G95A, T339E, Q343E, Q376R, and A414V; or K50N, H213N, L230I, T339E, Q343E, and L344M.

In some embodiments, the TS comprises relative to SEQ ID NO: 13: K50N, H213N, L230I, T339E, Q343E, and L344M; S100A, T339E, and Q343E; T339E, Q343E, L344M, and N527D; K50N, V1621, C180G, N196K, N211D, H213N, T339E, Q343E, and L344M; K50N, E150Q, V162I, C180G, N196K, N211 D, H213N, T339E, Q343E, and L344M; S116A, H213N, T339E, Q343E, L344M, and N527D; N196K, T339E, and Q343E; K50N, E150Q, V162L, A172P, C180G, N196K, N211D, H213N, T339E, Q343E, and L344M; V216L, T339E, and Q343E; S116A, H213N, T339E, Q343E, and N527D; S116A, T339E, Q343E, and N527D; or T339E, Q343E, and Q376P.

In some embodiments, the TS comprises a sequence that is at least 90%, at least 95%, at least 97%, at least 98%, at least 99% identical or is 100% identical to any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948, or a conservatively substituted version thereof.

Further aspects of the disclosure relate to non-naturally occurring TSs, wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14, and wherein the TS is capable of producing a CBC-type cannabinoid.

In some embodiments, relative to the sequence of SEQ ID NO: 14, the TS further comprises an amino acid substitution at one or more residues corresponding to positions 31, 40, 46, 74, 90, 255, 288, 290, 318, and/or 495 in SEQ ID NO: 14.

In some embodiments, the TS comprises: the amino acid Q at a residue corresponding to position 31 in SEQ ID NO: 14; the amino acid E at a residue corresponding to position 40 in SEQ ID NO: 14; the amino acid Y at a residue corresponding to position 41 in SEQ ID NO: 14; the amino acid P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid S at a residue corresponding to position 58 in SEQ ID NO: 14; the amino acid S at a residue corresponding to position 61 in SEQ ID NO: 14; the amino acid V or L at a residue corresponding to position 63 in SEQ ID NO: 14, the amino acid T at a residue corresponding to position 74 in SEQ ID NO: 14; the amino acid V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid V at a residue corresponding to position 255 in SEQ ID NO: 14; the amino acid M at a residue corresponding to position 257 in SEQ ID NO: 14; the amino acid E at a residue corresponding to position 268 in SEQ ID NO: 14; the amino acid V at a residue corresponding to position 273 in SEQ ID NO: 14; the amino acid L at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid E at a residue corresponding to position 340 in SEQ ID NO: 14; the amino acid Q at a residue corresponding to position 344 in SEQ ID NO: 14; the amino acid L at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid S at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I at a residue corresponding to position 442 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid I at a residue corresponding to position 489 in SEQ ID NO: 14; the amino acid I at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid F or P at a residue corresponding to position 494 in SEQ ID NO: 14; the amino acid E or K at a residue corresponding to position 495 in SEQ ID NO: 14; the amino acid N at a residue corresponding to position 496 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the TS comprises: the amino acid Y at a residue corresponding to position 41 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid S at a residue corresponding to position 58 in SEQ ID NO: 14; the amino acid S at a residue corresponding to position 61 in SEQ ID NO: 14, the amino acid V or L at a residue corresponding to position 63 in SEQ ID NO: 14; the amino acid G at a residue corresponding to position 95 in SEQ ID NO: 14; the amino acid S at a residue corresponding to position 96 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14, the amino acid R at a residue corresponding to position 136 in SEQ ID NO: 14; the amino acid E at a residue corresponding to position 143 in SEQ ID NO: 14; the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid M at a residue corresponding to position 257 in SEQ ID NO: 14; the amino acid E at a residue corresponding to position 268 in SEQ ID NO: 14; the amino acid V at a residue corresponding to position 273 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid E at a residue corresponding to position 340 in SEQ ID NO: 14; the amino acid Q at a residue corresponding to position 344 in SEQ ID NO: 14; the amino acid L at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid S at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I at a residue corresponding to position 442 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid I at a residue corresponding to position 489 in SEQ ID NO: 14; the amino acid I at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid F or P at a residue corresponding to position 494 in SEQ ID NO: 14; the amino acid N at a residue corresponding to position 496 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the TS comprises relative to SEQ ID NO: 14: Q58S, V288L, and F345L; R31Q, V52I, H56N, Q58S, M61S, I74T, N90V, A250P, S255V, F345L, Q475K, and T492N; R31Q, H56N, I74T, N90V, H143E, A250P, S255V, Q475K, and T492N; R31Q, H56N, I74T, N90V, A250P, S255V, L443I, Q475K, and T492N; H56N, M61S, N90V, A250D, S255V, V288L, Q475K, T492N, and A495E; R31Q, H56N, I74T, N90V, K215R, A250P, S255V, Q475K, and T492N; R31Q, P49A, H56N, Q58S, M61S, I74T, N90V, H143E, A250P, S255V, V288L, F345L, Q475K, and T492N; R31Q, A47T, H56N, I74T, N90V, A250P, S255V, Q475K, and T492N; M61 S, N90V, A250D, S255V, Q475K, T492N, A495E, and N498T; R31Q, H56N, M61S, I74T, N89H, N90V, S100A, H136R, E150Q, N196K, N211D, A250P, S255V, V288M, F345M, S382K, L443I, Q475K, and T492N; R31Q, H56N, Q58S, M61S, I74T, N90V, H143E, A250P, S255V, V288L, T340E, F345L, E424D, Q475K, and T492N; R31Q, H56N, I74T, S88L, N90V, A250P, S255V, Q475K, and T492N; R31Q, V52I, H56N, Q58S, M61S, I74T, N90V, H143E, A250P, S255V, V288L, F345L, Q475K, and T492N; R31Q, H56N, Q58S, M61S, I74T, N90V, H143E, A250P, S255V, V288L, T340E, F345L, E424D, Q475K, and T492N; R31Q, H56N, Q58S, M61S, 174T, N90V, H143E, A250P, S255V, V288L, F345L, A411V, Q475K, and T492N; R31Q, V52I, H56N, Q58S, M61S, 174T, N90V, A250P, S255V, V288L, F345L, Q475K, and T492N; R31Q, K50L, H56N, 174T, N90V, A250P, S255V, Q475K, and T492N; R31Q, A47T, V52I, H56N, Q58S, M61S, I74T, N90V, H143E, A250P, S255V, V288L, T340E, F345L, Q475K, and T492N; or R31Q, H56N, M61S, I74T, N89H, N90V, S100A, N196K, N211D, A250P, S255V, I257R, V288M, F345M, S382K, L443I, Q475K, and T492N.

In some embodiments, the TS comprises a sequence that is at least 90%, at least 95% at least 97%, at least 98%, at least 99% identical or is 100% identical to any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993, or a conservatively substituted version thereof.

Further aspects of the disclosure relate to non-naturally occurring nucleic acids encoding a TS, wherein the non-naturally occurring nucleic acid comprises a sequence that is at least 90%, at least 95% at least 97%, at least 98%, or at least 99% identical to any one of SEQ ID NOs: 46-134, 194-222, 322-463, 954-1189, 1195-1197, 1201, 1202, and 1204. In some embodiments, the non-naturally occurring nucleic acid comprises the sequence of any one of SEQ ID NOs: 46-134, 194-222, 322-463, 954-1189, 1195-1197, 1201, 1202, and 1204, or a conservatively substituted version thereof.

Further aspects of the disclosure relate to vectors comprising non-naturally occurring nucleic acids associated with the disclosure.

Further aspects of the disclosure relate to expression cassettes comprising non-naturally occurring nucleic acids associated with the disclosure.

Further aspects of the disclosure relate to host cells transformed with non-naturally occurring nucleic acids, vectors, or expression cassettes associated with the disclosure.

Further aspects of the disclosure relate to bioreactors for producing a cannabinoid, wherein the bioreactor contains a CBG-type cannabinoid and a TS, wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14, wherein the TS does not comprise the sequence of SEQ ID NO: 20, 21, 320 or 321.

Further aspects of the disclosure relate to bioreactors for producing a cannabinoid, wherein the bioreactor contains a CBG-type cannabinoid and a TS, wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168, 171, 172,175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13.

Further aspects of the disclosure relate to bioreactors for producing a cannabinoid, wherein the bioreactor contains a CBG-type cannabinoid and a TS, wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14, wherein the TS is capable of producing more of a CBC-type cannabinoid than a control TS, and wherein the control TS comprises the sequence of SEQ ID NO: 21.

Further aspects of the disclosure relate to bioreactors for producing a cannabinoid, wherein the bioreactor contains a CBG-type cannabinoid and any of the host cells associated with the disclosure.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting the native Cannabis biosynthetic pathway for production of cannabinoid compounds, including five enzymatic steps mediated by: (R1a) acyl activating enzymes (AAE); (R2a) olivetol synthase enzymes (OLS); (R3a) olivetolic acid cyclase enzymes (OAC); (R4a) cannabigerolic acid synthase enzymes (CBGAS); and (R5a) terminal synthase enzymes (TS). Formulae 1a-11a correspond to hexanoic acid (1a), hexanoyl-CoA (2a), malonyl-CoA (3a), 3,5,7-trioxododecanoyl-CoA (4a), olivetol (5a), olivetolic acid (6a), geranyl pyrophosphate (7a), cannabigerolic acid (8a), cannabidiolic acid (9a), tetrahydrocannabinolic acid (10a), and cannabichromenic acid (11a). Hexanoic acid is an exemplary carboxylic acid substrate; other carboxylic acids may also be used (e.g., butyric acid, isovaleric acid, octanoic acid, decanoic acid, etc.; see e.g., FIG. 3 below). The enzymes that catalyze the synthesis of 3,5,7-trioxododecanoyl-CoA and olivetolic acid are shown in R2a and R3a, respectively, and can include multi-functional enzymes that catalyze the synthesis of 3,5,7-trioxododecanoyl-CoA and olivetolic acid. The enzymes cannabidiolic acid synthase (CBDAS), tetrahydrocannabinolic acid synthase (THCAS), and cannabichromenic acid synthase (CBCAS) that catalyze the synthesis of cannabidiolic acid, tetrahydrocannabinolic acid, and cannabichromenic acid, respectively, are shown in step R5a. FIG. 1 is adapted from Carvalho et al. “Designing Microorganisms for Heterologous Biosynthesis of Cannabinoids” (2017) FEMS Yeast Research June 1; 17(4), which is incorporated by reference in its entirety.

FIG. 2 is a schematic depicting a heterologous biosynthetic pathway for production of cannabinoid compounds, including five enzymatic steps mediated by: (R1) acyl activating enzymes (AAE); (R2) polyketide synthase enzymes (PKS) or bifunctional polyketide synthase-polyketide cyclase enzymes (PKS-PKC); (R3) polyketide cyclase enzymes (PKC) or bifunctional PKS-PKC enzymes; (R4) prenyltransferase enzymes (PT); and (R5) terminal synthase enzymes (TS). Any carboxylic acid of varying chain lengths, structures (e.g., aliphatic, alicyclic, or aromatic) and functionalization (e.g., hydroxylic-, keto-, amino-, thiol-, aryl-, or alogeno-) may also be used as precursor substrates (e.g., thiopropionic acid, hydroxy phenyl acetic acid, norleucine, bromodecanoic acid, butyric acid, isovaleric acid, octanoic acid, decanoic acid, etc).

FIG. 3 is a non-exclusive representation of select putative precursors for the cannabinoid pathway in FIG. 2.

FIG. 4 is a schematic showing a reaction catalyzed by a TS enzyme wherein the geranyl moiety of cannabigerolic acid (Formula (8a)) is cyclized to yield cannabidiolic acid, tetrahydrocannabinolic acid, or cannabichromenic acid.

FIG. 5 is a schematic showing a plasmid bearing the transcriptional unit encoding a TS. The coding sequence for the candidate TS enzymes in the libraries (labeled “Terminal Synthase”) was driven by the GAL1 promoter. Each candidate TS enzyme possessed an N-terminally fused signal peptide (labeled “N-terminal signal peptide”) and a C-terminally fused signal peptide (labeled “C-terminal signal peptide”).

FIG. 6 depicts a graph showing tetrahydrocannabinolic acid (THCA) titers of THCAS enzymes fused with various N- and C-terminal signal peptides depicted on the X-axis. The strains included in FIG. 6, listed from left to right are: 631201 (containing signal peptide UBC6), 631191 (containing signal peptides YLR120C and HDEL), 631195 (containing signal peptides Osm1p and HDEL), 631199 (containing signal peptides Ost1 leader and HDEL), 631208 (containing signal peptide Ost1 leader), 631190 (containing signal peptides Mfa2 and HDEL), 631197 (containing signal peptides Sf leader and HDEL), 631188 (containing signal peptide HDEL), 631211 (containing signal peptide ERG11-leader), 631193 (containing signal peptides Mfa2 and HDEL), 631207 (containing signal peptide Mfa2), 631216 (containing signal peptide Mfa2), 631203 (containing signal peptide CVIA from Mfa), 631192 (containing signal peptides YLR120C and KLD), 631196 (containing signal peptides Osm1p and KLD), 631200 (containing signal peptides Ost1 leader and KLD), 631194 (containing signal peptides Mfa2 and KLD), 631198 (containing signal peptides Sf leader and KLD), 631212 (containing signal peptide Osm1), 631205 (containing signal peptide YNL121C), 631210 (containing signal peptide OSM1-leader-T23L), 631202 (containing signal peptide PTS1 SKL), 631206 (containing signal peptide YLR120C), 631215 (containing signal peptide PEP4 (long (2-76)), 631204 (containing signal peptide YDR456W), 631189 (containing signal peptide Komagataella phaffii PEP4), 631213 (containing signal peptide PRC1-11), 631214 (containing signal peptide PEP4 (short (1-24)), 631209 (containing signal peptide Sf leader), and 631185 (GFP negative control). Strain IDs and their corresponding activity from this graph are shown in Table 6.

FIG. 7 depicts a graph showing screening activity data of candidate TS enzymes identified in Example 2 for THCA production based on an in vivo activity assay in S. cerevisiae. Strain t616313, expressing GFP, was used as a negative control. The data show the plotting of four bioreplicates. Strain IDs and their corresponding activity from this graph are shown in Table 8.

FIG. 8 depicts a graph showing screening activity data of candidate TS enzymes identified in Example 2 for cannabidiolic acid (CBDA) production based on an in vivo activity assay in S. cerevisiae. Strain t616314, expressing a Cannabis CBDAS, was used as a positive control for determining hit ranking of the library members. Strain t616313, expressing GFP, was used as a negative control. The data show the plotting of four bioreplicates. Strain IDs and their corresponding activity from this graph are shown in Table 9.

FIG. 9 depicts a graph showing screening activity data of candidate TS enzymes identified in Example 2 for cannabichromenic acid (CBCA) production based on an in vivo activity assay in S. cerevisiae. Strain t616313, expressing GFP, was used as a negative control. The data show the plotting of four bioreplicates. Strain IDs and their corresponding activity from this graph are shown in Table 10.

FIG. 10 depicts a graph showing screening activity data of candidate TS enzymes identified in Example 3 for THCA production based on an in vivo activity assay in S. cerevisiae. Strain t701870, expressing a Cannabis THCAS, was used as a positive control and for determining hit ranking of the library members. Strain t616313, expressing GFP, was used as a negative control. The data show the plotting of two bioreplicates. Strain IDs and their corresponding activity from this graph are shown in Table 11.

FIG. 11 depicts a graph showing screening activity data of candidate TS enzymes identified in Example 3 for CBDA production based on an in vivo activity assay in S. cerevisiae. Strain t616314, expressing a Cannabis CBDAS, was used as a positive control for determining hit ranking of the library members. Strain t616313, expressing GFP, was used as a negative control. The data show the plotting of two bioreplicates. Strain IDs and their corresponding activity from this graph are shown in Table 12.

FIG. 12 depicts a graph showing screening activity data of candidate TS enzymes identified in Example 3 for CBCA production based on an in vivo activity assay in S. cerevisiae. Strain t616315, expressing a Cannabis THCAS, was used as a positive control and for determining hit ranking of the library members. Strain t616313, expressing GFP, was used as a negative control. The data show the plotting of two bioreplicates. Strain IDs and their corresponding activity from this graph are shown in Table 13.

FIGS. 13A-13C depict graphs showing screening activity data of candidate TS enzymes identified in Example 4 for THCA, CBDA, and CBCA production based on an in vivo activity assay in S. cerevisiae. Strain t807949, expressing a C. sativa THCAS, strain t820182, expressing a C. sativa THCAS, and strain t807973, expressing a C. sativa CBDAS, were used as positive controls and for determining hit ranking of the library members. Strain t807914, expression GFP, was used as a negative control. The data show the plotting of four bioreplicates. FIG. 13A depicts THCA production. FIG. 13B depicts CBDA production. FIG. 13C depicts CBCA production. Strains depicted in FIGS. 13A-13C and their corresponding activity are shown in Table 14.

FIGS. 14A-14C depict graphs showing screening activity data of candidate TS enzymes identified in Example 4 for THCVA, CBDVA, and CBCVA production based on an in vivo activity assay in S. cerevisiae. Strain t807949, expressing a C. sativa THCAS, strain t820182, expressing a C. sativa THCAS, and strain t807973, expressing a C. sativa CBDAS were used as positive controls and for determining hit ranking of the library members. Strain t807914, expression GFP, was used as a negative control. The data show the plotting of four bioreplicates. FIG. 14A depicts THCVA production. FIG. 14B depicts CBDVA production.

FIG. 14C depicts CBCVA production. Strains depicted in FIGS. 14A-14C and their corresponding activity are shown in Table 15.

FIG. 15 depicts a graph showing screening activity data of candidate TS enzymes identified in Example 5 for THCA production based on an in vivo activity assay in S. cerevisiae. Strain 876606, expressing a C. sativa THCAS, was used as a positive control for THCAS activity. Strain 865977, expressing a THCAS candidate from Example 4, was also used as a positive control for determining hit ranking of the library members. Strains engineered to produce THCA were normalized to the in-plate performance of strain 865977. Strains depicted in FIG. 15 and their corresponding activity are shown in Table 16.

FIG. 16 depicts a graph showing screening activity data of candidate TS enzymes identified in Example 5 for CBDA production based on an in vivo activity assay in S. cerevisiae. Strain 876607, expressing a C. sativa CBDAS, was used as a positive control for THCAS activity. Strain 865859, expressing a CBDAS candidate from Example 4, was also used as a positive control for determining hit ranking of the library members. Strains engineered to produce CBDA were normalized to the in-plate performance of strain 865859. Strains depicted in FIG. 16 and their corresponding activity are shown in Table 16.

FIG. 17 depicts a graph showing screening activity data of candidate TS enzymes identified in Example 5 for CBCA production based on an in vivo activity assay in S. cerevisiae. Strain 876607 expressing a C. sativa CBDAS, and strain 865977, expressing a THCAS candidate from Example 4, were used as controls. Strains depicted in FIG. 17 and their corresponding activity are shown in Table 16.

### DETAILED DESCRIPTION

This disclosure provides methods for production of cannabinoids and cannabinoid precursors from fatty acid substrates using genetically modified host cells. Methods include heterologous expression of a terminal synthase (TS), such as a tetrahydrocannabinolic acid synthase (THCAS), a cannabidiolic acid synthase (CBDAS), and/or a cannabichromenic acid synthase (CBCAS). The application describes TSs that can be functionally expressed in host cells such as S. cerevisiae. As demonstrated in the Examples, multiple THCAS, CBDAS and CBCAS enzymes were identified that were capable of producing tetrahydrocannabinolic acid (THCA), tetrahydrocannabivarin acid (THCVA), cannabidiolic acid (CBDA), cannabidivarinic acid (CBDVA), cannabichromenic acid (CBCA), and/or cannabichromevarinic acid (CBCVA) in a host cell. The TSs described in this disclosure may be useful in increasing the efficiency and purity of cannabinoid production, such as, for example, by altering the activity and/or abundance of such enzymes.

### Definitions

While the following terms are believed to be well understood by one of ordinary skill in the art, the following definitions are set forth to facilitate explanation of the disclosed subject matter.

The term “a” or “an” refers to one or more of an entity, i.e., can identify a referent as plural. Thus, the terms “a” or “an,” “one or more” and “at least one” are used interchangeably in this application. In addition, reference to “an element” by the indefinite article “a” or “an” does not exclude the possibility that more than one of the elements is present, unless the context clearly requires that there is one and only one of the elements.

The terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure may refer to the “microorganisms” or “microbes” of lists/tables and figures present in the disclosure. This characterization can refer to not only the identified taxonomic genera of the tables and figures, but also the identified taxonomic species, as well as the various novel and newly identified or designed strains of any organism in the tables or figures. The same characterization holds true for the recitation of these terms in other parts of the specification, such as in the Examples.

The term “prokaryotes” is recognized in the art and refers to cells that contain no nucleus or other cell organelles. The prokaryotes are generally classified in one of two domains, the Bacteria and the Archaea.

“Bacteria” or “eubacteria” refers to a domain of prokaryotic organisms. Bacteria include at least 11 distinct groups as follows: (1) Gram-positive (gram+) bacteria, of which there are two major subdivisions: (a) high G+C group (Actinomycetes, Mycobacteria, Micrococcus, others) and (b) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

The term “Archaea” refers to a taxonomic classification of prokaryotic organisms with certain properties that make them distinct from Bacteria in physiology and phylogeny.

The term “Cannabis” refers to a genus in the family Cannabaceae. Cannabis is a dioecious plant. Glandular structures located on female flowers of Cannabis, called trichomes, accumulate relatively high amounts of a class of terpeno-phenolic compounds known as phytocannabinoids (described in further detail below). Cannabis has conventionally been cultivated for production of fibre and seed (commonly referred to as “hemp-type”), or for production of intoxicants (commonly referred to as “drug-type”). In drug-type Cannabis, the trichomes contain relatively high amounts of tetrahydrocannabinolic acid (THCA), which can convert to tetrahydrocannabinol (THC) via a decarboxylation reaction, for example upon combustion of dried Cannabis flowers, to provide an intoxicating effect. Drug-type Cannabis often contains other cannabinoids in lesser amounts. In contrast, hemp-type Cannabis contains relatively low concentrations of THCA, often less than 0.3% THC by dry weight. Hemp-type Cannabis may contain non-THC and non-THCA cannabinoids, such as cannabidiolic acid (CBDA), cannabidiol (CBD), and other cannabinoids. Presently, there is a lack of consensus regarding the taxonomic organization of the species within the genus. Unless context dictates otherwise, the term “Cannabis” is intended to include all putative species within the genus, such as, without limitation, Cannabis sativa, Cannabis indica, and Cannabis ruderalis and without regard to whether the Cannabis is hemp-type or drug-type.

The term “cyclase activity” in reference to a polyketide synthase (PKS) enzyme (e.g., an olivetol synthase (OLS) enzyme) or a polyketide cyclase (PKC) enzyme (e.g., an olivetolic acid cyclase (OAC) enzyme), refers to the activity of catalyzing the cyclization of an oxo fatty acyl-CoA (e.g., 3,5,7-trioxododecanoyl-COA, 3,5,7-trioxodecanoyl-COA) to the corresponding intramolecular cyclization product (e.g., olivetolic acid, divarinic acid). In some embodiments, the PKS or PKC catalyzes the C2-C7 aldol condensation of an acyl-COA with three additional ketide moieties added thereto.

A “cytosolic” or “soluble” enzyme refers to an enzyme that is predominantly localized (or predicted to be localized) in the cytosol of a host cell.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (i.e., bacteria and archaea) is that they have membrane-bound organelles, especially the nucleus, which contains the genetic material, and is enclosed by the nuclear envelope.

The term “host cell” refers to a cell that can be used to express a polynucleotide, such as a polynucleotide that encodes an enzyme used in biosynthesis of cannabinoids or cannabinoid precursors. The terms “genetically modified host cell,” “recombinant host cell,” and “recombinant strain” are used interchangeably and refer to host cells that have been genetically modified by, e.g., cloning and transformation methods, or by other methods known in the art (e.g., selective editing methods, such as CRISPR). Thus, the terms include a host cell (e.g., bacterial cell, yeast cell, fungal cell, insect cell, plant cell, mammalian cell, human cell, etc.) that has been genetically altered, modified, or engineered, so that it exhibits an altered, modified, or different genotype and/or phenotype, as compared to the naturally-occurring cell from which it was derived. It is understood that in some embodiments, the terms refer not only to the particular recombinant host cell in question, but also to the progeny or potential progeny of such a host cell.

The term “control host cell,” or the term “control” when used in relation to a host cell, refers to an appropriate comparator host cell for determining the effect of a genetic modification or experimental treatment. In some embodiments, the control host cell is a wild type cell. In other embodiments, a control host cell is genetically identical to the genetically modified host cell, except for the genetic modification(s) differentiating the genetically modified or experimental treatment host cell. In some embodiments, the control host cell has been genetically modified to express a wild type or otherwise known variant of an enzyme being tested for activity in other test host cells.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez el al., Nat Methods. 2016 July; 13(7). 563-567. A heterologous polynucleotide may comprise a wild-type sequence or a mutant sequence as compared with a reference polynucleotide sequence.

The term “at least a portion” or “at least a fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of an enzyme, such as a catalytic domain. A biologically active portion of a genetic regulatory element may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence promotes transcription of the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

The terms “link,” “linked,” or “linkage” means two entities (e.g., two polynucleotides or two proteins) are bound to one another by any physicochemical means. Any linkage known to those of ordinary skill in the art, covalent or non-covalent, is embraced. In some embodiments, a nucleic acid sequence encoding an enzyme of the disclosure is linked to a nucleic acid encoding a signal peptide. In some embodiments, an enzyme of the disclosure is linked to a signal peptide. Linkage can be direct or indirect.

The terms “transformed” or “transform” with respect to a host cell refer to a host cell in which one or more nucleic acids have been introduced, for example on a plasmid or vector or by integration into the genome. In some instances where one or more nucleic acids are introduced into a host cell on a plasmid or vector, one or more of the nucleic acids, or fragments thereof, may be retained in the cell, such as by integration into the genome of the cell, while the plasmid or vector itself may be removed from the cell. In such instances, the host cell is considered to be transformed with the nucleic acids that were introduced into the cell regardless of whether the plasmid or vector is retained in the cell or not.

The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M·T⁻¹·M⁻¹ or M·T⁻¹·L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be generated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all products of interest produced in a process, including but not limited to the products of interest in solution, the products of interest in gas phase if applicable, and any products of interest removed from the process and recovered relative to the initial volume in the process or the operating volume in the process. For example, the total titer of products of interest (e.g., small molecule, peptide, synthetic compound, fuel, alcohol, etc.) in a fermentation broth is described as g of products of interest in solution per liter of fermentation broth or cell-free broth (g/L) or as g of products of interest in solution per kg of fermentation broth or cell-free broth (g/Kg).

The term “amino acid” refers to organic compounds that comprise an amino group, —NH2, and a carboxyl group, —COOH. The term “amino acid” includes both naturally occurring and unnatural amino acids. Nomenclature for the twenty common amino acids is as follows: alanine (ala or A); arginine (arg or R); asparagine (asn or N); aspartic acid (asp or D); cysteine (cys or C); glutamine (gln or Q); glutamic acid (glu or E); glycine (gly or G); histidine (his or H); isoleucine (ile or I); leucine (leu or L); lysine (lys or K); methionine (met or M); phenylalanine (phe or F); proline (pro or P); serine (ser or S); threonine (thr or T); tryptophan (trp or W); tyrosine (tyr or Y); and valine (val or V). Non-limiting examples of unnatural amino acids include homo-amino acids, proline and pyruvic acid derivatives, 3-substituted alanine derivatives, glycine derivatives, ring-substituted phenylalanine derivatives, ring-substituted tyrosine derivatives, linear core amino acids, amino acids with protecting groups including Fmoc, Boc, and Cbz, β-amino acids (β3 and β2), and N-methyl amino acids.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of, or a substituent that is, a straight-chain or branched saturated hydrocarbon group having from 1 to 20 carbon atoms (“C1-20 alkyl”). In certain embodiments, the term “alkyl” refers to a radical of, or a substituent that is, a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 2 to 7 carbon atoms (“C2-7 alkyl”). In some embodiments, an alkyl group has 3 to 7 carbon atoms (“C3-7 alkyl”). In some embodiments, an alkyl group has 1 to 6 carbon atoms (“C₁₋₆ alkyl”). In some embodiments, an alkyl group has 2 to 6 carbon atoms (“C₂₋₆ alkyl”). In some embodiments, an alkyl group has 3 to 5 carbon atoms (“C₃₋₅ alkyl”). In some embodiments, an alkyl group has 5 carbon atoms (“C₅ alkyl”). In some embodiments, the alkyl group has 3 carbon atoms (“C3 alkyl”). In some embodiments, the alkyl group has 7 carbon atoms (“C7 alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”).

Examples of C₁₋₆ alkyl groups include methyl (C₁), ethyl (C₂), propyl (C₃) (e.g., n-propyl, isopropyl), butyl (C₄)(e.g., n-butyl, tert-butyl, sec-butyl, iso-butyl), pentyl (C₅)(e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, benzyl).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described in this application that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkenyl” refers to a radical of, or a substituent that is, a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₄ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of, or a substituent that is, a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl. In certain embodiments, the aralkyl is 7-phenylheptanyl. In certain embodiments, the aralkyl is C7 alkyl substituted by an optionally substituted aryl group (e.g., phenyl). In certain embodiments, the aralkyl is a C7-C10 alkyl group substituted by an optionally substituted aryl group (e.g., phenyl).

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined in this application. Likewise, “saturated” refers to a group that does not contain a double or triple bond, i.e., contains all single bonds.

The term “optionally substituted” means substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described in this application that results in the formation of a stable compound. The present invention contemplates any and all such combinations in order to arrive at a stable compound. For purposes of this invention, heteroatoms such as nitrogen may have hydrogen substituents and/or any suitable substituent as described in this application which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NRC(═O)N(R^{bb}), —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl;

wherein;
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion. Alternatively, two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
   wherein:
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated by reference. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of a compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (1), (9), (10), and (11) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and described by the R- and S-sequencing rules of Cahn and Prelog. An enantiomer can also be characterized by the manner in which the molecule rotates the plane of polarized light, and designated as dextrorotatory or levorotatory (i.e., as (+) or (−)-isomers respectively). A chiral compound can exist as either an individual enantiomer or as a mixture of enantiomers. A mixture containing equal proportions of the enantiomers is called a “racemic mixture.”

The term “co-crystal” refers to a crystalline structure comprising at least two different components (e.g., a compound described in this application and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound described in this application is complexed with the acid in a way that proton transfer (e.g., a complete proton transfer) from the acid to a compound described in this application easily occurs at room temperature. In the co-crystal, however, a compound described in this application is complexed with the acid in a way that proton transfer from the acid to a compound described in this application does not easily occur at room temperature. In certain embodiments, in the co-crystal, there is no proton transfer from the acid to a compound described in this application. In certain embodiments, in the co-crystal, there is partial proton transfer from the acid to a compound described in this application. Co-crystals may be useful to improve the properties (e.g., solubility, stability, and ease of formulation) of a compound described in this application.

The term “polymorphs” refers to a crystalline form of a compound (or a salt, hydrate, or solvate thereof) in a particular crystal packing arrangement. All polymorphs of the same compound have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrug” refers to compounds, including derivatives of the compounds of Formula (X), (8), (9), (10), or (11), that have cleavable groups and become by solvolysis or under physiological conditions the compounds of Formula (X), (8), (9), (10), or (11) and that are pharmaceutically active in vivo. The prodrugs may have attributes such as, without limitation, solubility, bioavailability, tissue compatibility, or delayed release in a mammalian organism. Examples include, but are not limited to, derivatives of compounds described in this application, including derivatives formed from glycosylation of the compounds described in this application (e.g., glycoside derivatives), carrier-linked prodrugs (e.g., ester derivatives), bioprecursor prodrugs (a prodrug metabolized by molecular modification into the active compound), and the like. Non-limiting examples of glycoside derivatives are disclosed in and incorporated by reference from PCT Publication No. WO2018/208875 and U.S. Patent Publication No. 2019/0078168. Non-limiting examples of ester derivatives are disclosed in and incorporated by reference from U.S. Patent Publication No. US2017/0362195.

Other derivatives of the compounds of this invention have activity in both their acid and acid derivative forms, but the acid sensitive form often offers advantages of solubility, bioavailability, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formula (X), (8), (9), (10), or (11) may be preferred.

### Cannabinoids

As used in this application, the term “cannabinoid” includes compounds of Formula (X):

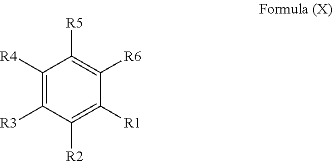

or a pharmaceutically acceptable salt, co-crystal, tautomer, stereoisomer, solvate, hydrate, polymorph, isotopically enriched derivative, or prodrug thereof, wherein R1 is hydrogen, optionally substituted acyl, optionally substituted alkyl, optionally substituted alkenyl, optionally substituted alkynyl, optionally substituted carbocyclyl, or optionally substituted aryl; R2 and R6 are, independently, hydrogen or carboxyl; R3 and R5 are, independently, hydroxyl, halogen, or alkoxy; and R4 is a hydrogen or an optionally substituted prenyl moiety; or optionally R4 and R3 are taken together with their intervening atoms to form a cyclic moiety, or optionally R4 and R5 are taken together with their intervening atoms to form a cyclic moiety, or optionally both 1) R4 and R3 are taken together with their intervening atoms to form a cyclic moiety and 2) R4 and R5 are taken together with their intervening atoms to form a cyclic moiety. In certain embodiments, R4 and R3 are taken together with their intervening atoms to form a cyclic moiety. In certain embodiments, R4 and R5 are taken together with their intervening atoms to form a cyclic moiety. In certain embodiments, “cannabinoid” refers to a compound of Formula (X), or a pharmaceutically acceptable salt thereof. In certain embodiments, both 1) R4 and R3 are taken together with their intervening atoms to form a cyclic moiety and 2) R4 and R5 are taken together with their intervening atoms to form a cyclic moiety.

In some embodiments, cannabinoids may be synthesized via the following steps: a) one or more reactions to incorporate three additional ketone moieties onto an acyl-CoA scaffold, where the acyl moiety in the acyl-CoA scaffold comprises between four and fourteen carbons; b) a reaction cyclizing the product of step (a); and c) a reaction to incorporate a prenyl moiety to the product of step (b) or a derivative of the product of step (b). In some embodiments, non-limiting examples of the acyl-CoA scaffold described in step (a) include hexanoyl-CoA and butyryl-CoA. In some embodiments, non-limiting examples of the product of step (b) or a derivative of the product of step (b) include olivetolic acid, divarinic acid, and sphaerophorolic acid.

In some embodiments, a cannabinoid compound of Formula (X) is of Formula (X-A), (X-B), or (X-C):

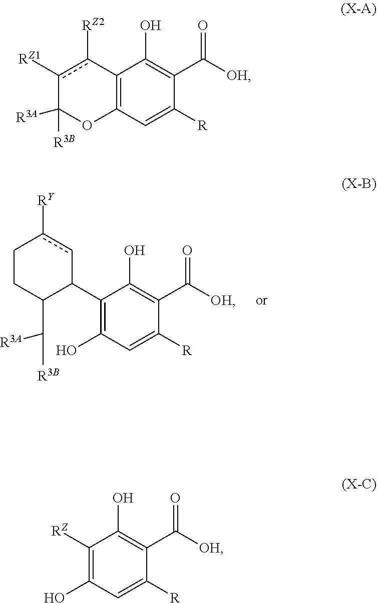

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;
R is hydrogen, optionally substituted acyl, optionally substituted alkyl, optionally substituted alkenyl, optionally substituted alkynyl, optionally substituted carbocyclyl, or optionally substituted aryl;
R^{Z1} is hydrogen, optionally substituted acyl, optionally substituted alkyl, optionally substituted alkenyl, optionally substituted alkynyl, optionally substituted carbocyclyl, or optionally substituted aryl;
R^{Z2} is hydrogen, optionally substituted acyl, optionally substituted alkyl, optionally substituted alkenyl, optionally substituted alkynyl, optionally substituted carbocyclyl, or optionally substituted aryl;
or optionally, R^{Z1} and R^{Z2} are taken together with their intervening atoms to form an optionally substituted carbocyclic ring;
R^{3A} is hydrogen, optionally substituted acyl, optionally substituted alkyl, optionally substituted alkenyl, or optionally substituted alkynyl;
R^{3B} is hydrogen, optionally substituted acyl, optionally substituted alkyl, optionally substituted alkenyl, or optionally substituted alkynyl;
R^{Y} is hydrogen, optionally substituted acyl, optionally substituted alkyl, optionally substituted alkenyl, or optionally substituted alkynyl;
R^{Z} is hydrogen, optionally substituted acyl, optionally substituted alkyl, optionally substituted alkenyl, or optionally substituted alkynyl.

In certain embodiments, a cannabinoid compound is of Formula (X-A):

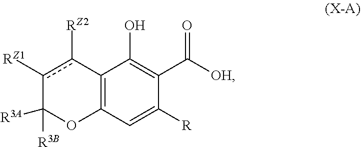

wherein [symbol] is a double bond, and each of R^{Z1} and R^{Z2} is hydrogen, one of R^{3A} and R^{3B} is optionally substituted C₂₋₆ alkenyl, and the other one of R^{3A} and R^{3B} is optionally substituted C₂₋₆ alkyl. In some embodiments, a cannabinoid compound of Formula (X) is of Formula (X-A), wherein each of R^{Z1} and R^{Z2} is hydrogen, one of R^{3A} and R^{3B} is a prenyl group, and the other one of R^{3A} and R^{3B} is optionally substituted methyl.

In certain embodiments, a cannabinoid compound of Formula (X) of Formula (X-A) is of Formula (11-z):

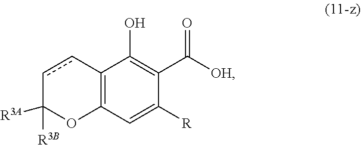

wherein [symbol] is a double bond or single bond, as valency permits; one of R^{3A} and R^{3B} is C₁₋₆ alkyl optionally substituted with alkenyl, and the other of R^{3A} and R^{3B} is optionally substituted C₁₋₆ alkyl. In certain embodiments, in a compound of Formula (11-z), [symbol] is a single bond; one of R^{3A} and R^{3B} is C₁₋₆ alkyl optionally substituted with prenyl; and the other of one of R^{3A} and R^{3B} is unsubstituted methyl; and R is as described in this application. In certain embodiments, in a compound of Formula (11-z), [symbol] is a single bond; one of R^{3A} and R^{3B} is

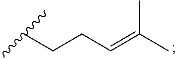 [SMILES: *CCC=C(C)C]

and the other of one of R^{3A} and R^{3B} is unsubstituted methyl; and R is as described in this application. In certain embodiments, a cannabinoid compound of Formula (11-z) is of Formula (11a):

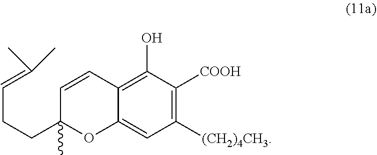

In certain embodiments, a cannabinoid compound of Formula (X) of Formula (X-A) is of Formula (11a):

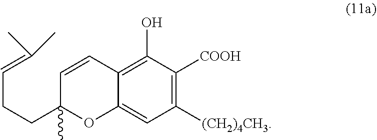

In certain embodiments, a cannabinoid compound of Formula (X-A) is of Formula (10-z):

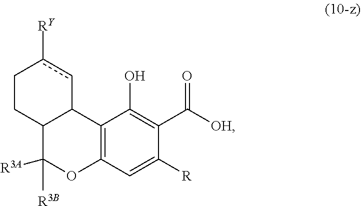

wherein [symbol] is a double bond or single bond, as valency permits; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (10-z), [symbol] is a single bond; each of R^{3A} and R^{3B} is unsubstituted methyl, and R is as described in this application. In certain embodiments, a cannabinoid compound of Formula (10-z) is of Formula (10a):

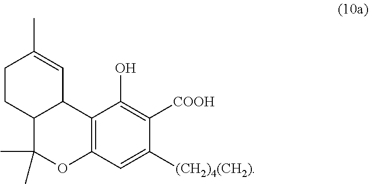

In certain embodiments, a compound of Formula (10a)

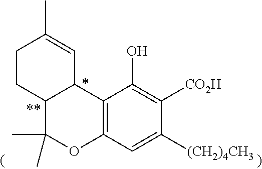

has a chiral atom labeled with * at carbon 10 and a chiral atom labeled with ** at carbon 6. In certain embodiments, in a compound of Formula (10a)

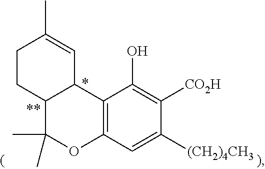

the chiral atom labeled with * at carbon 10 is of the R-configuration or S-configuration; and a chiral atom labeled with ** at carbon 6 is of the R-configuration. In certain embodiments, in a compound of Formula (10a)

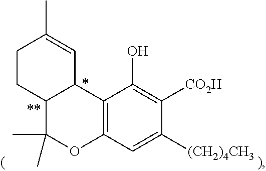

the chiral atom labeled with * at carbon 10 is of the S-configuration; and a chiral atom labeled with ** at carbon 6 is of the R-configuration or S-configuration. In certain embodiments, in a compound of Formula (10a)

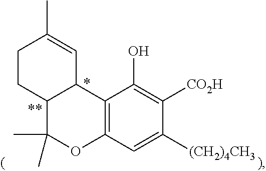

the chiral atom labeled with * at carbon 10 is of the R-configuration and a chiral atom labeled with ** at carbon 6 is of the R-configuration. In certain embodiments, a compound of Formula (10a)

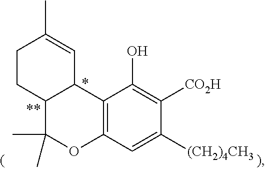

is of the formula:

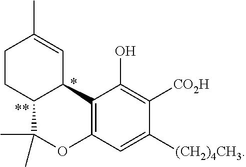

In certain embodiments, in a compound of Formula (10a)

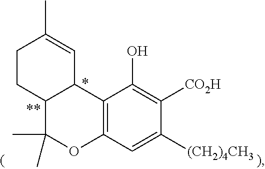

the chiral atom labeled with * at carbon 10 is of the S-configuration and a chiral atom labeled with ** at carbon 6 is of the S-configuration. In certain embodiments, a compound of Formula (10a)

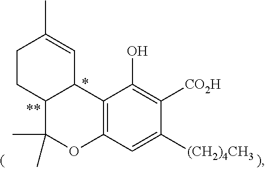

is of the formula:

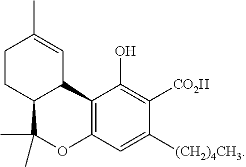

In certain embodiments, a cannabinoid compound is of Formula (X-B):

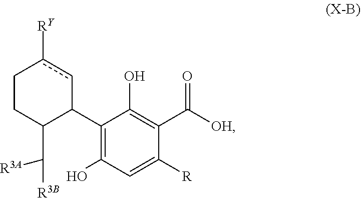

wherein [symbol] is a double bond; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (X-B), R^{Y} is optionally substituted C₁₋₆ alkyl; one of R^{3A} and R^{3B} is [symbol] ; and the other one of R^{3A} and R^{3B} is unsubstituted methyl, and R is as described in this application. In certain embodiments, a compound of Formula (X-B) is of Formula (9a):

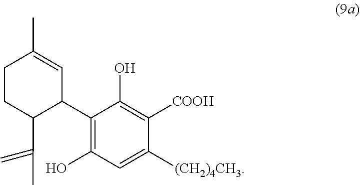

In certain embodiments, a compound of Formula (9a)

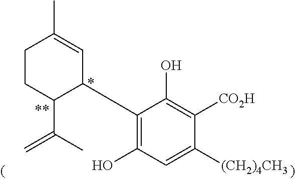

has a chiral atom labeled with * at carbon 3 and a chiral atom labeled with ** at carbon 4. In certain embodiments, in a compound of Formula (9a)

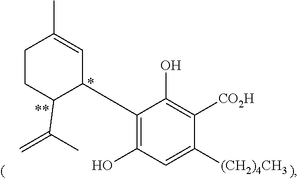

the chiral atom labeled with * at carbon 3 is of the R-configuration or S-configuration; and a chiral atom labeled with ** at carbon 4 is of the R-configuration. In certain embodiments, in a compound of Formula (9a)

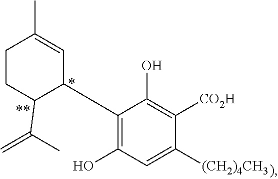

the chiral atom labeled with * at carbon 3 is of the S-configuration; and a chiral atom labeled with ** at carbon 4 is of the R-configuration or S-configuration. In certain embodiments, in a compound of Formula (9a)

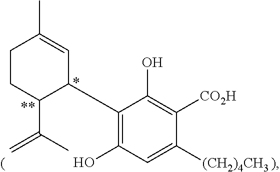

the chiral atom labeled with * at carbon 3 is of the R-configuration and a chiral atom labeled with ** at carbon 4 is of the R-configuration. In certain embodiments, a compound of Formula (9a)

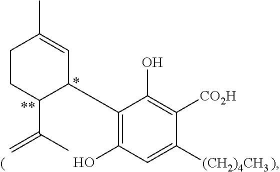

is of the formula:

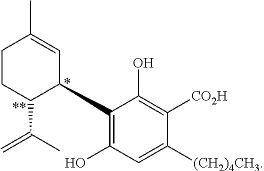

In certain embodiments, in a compound of Formula (9a)

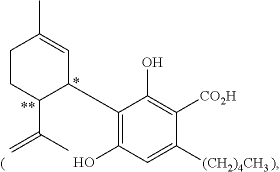

the chiral atom labeled with * at carbon 3 is of the S-configuration and a chiral atom labeled with ** at carbon 4 is of the S-configuration. In certain embodiments, a compound of Formula (9a)

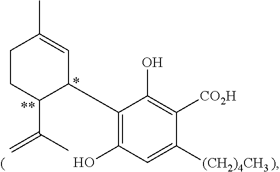

is of the formula:

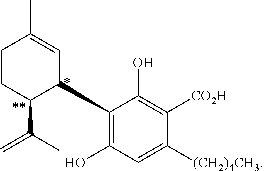

In certain embodiments, a cannabinoid compound is of Formula (X-C):

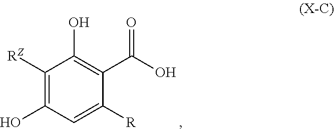

wherein R^{Z} is optionally substituted alkyl or optionally substituted alkenyl. In certain embodiments, a compound of Formula (X-C) is of formula:

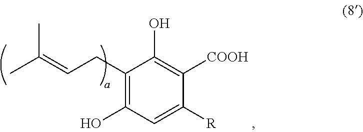

wherein a is 1, 2, 3, 4, 5, 6, 7, 8, 9, or 10. In certain embodiments, a is 1. In certain embodiments, a is 2. In certain embodiments, a is 3. In certain embodiments, a is 1, 2, or 3 for a compound of Formula (X-C). In certain embodiments, a cannabinoid compound is of Formula (X-C), and a is 1, 2, 3, 4, or 5. In certain embodiments, a compound of Formula (X-C) is of Formula (8a):

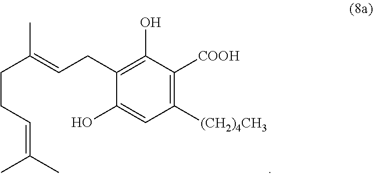

In some embodiments, cannabinoids of the present disclosure comprise cannabinoid receptor ligands. Cannabinoid receptors are a class of cell membrane receptors in the G protein-coupled receptor superfamily. Cannabinoid receptors include the CB₁ receptor and the CB₂ receptor. In some embodiments, cannabinoid receptors comprise GPR18, GPR55, and PPAR. (See Bram et al. “Activation of GPR18 by cannabinoid compounds: a tale of biased agonism” Br J Pharmcol v171 (16) (2014); Shi et al. “The novel cannabinoid receptor GPR55 mediates anxiolytic-like effects in the medial orbital cortex of mice with acute stress” Molecular Brain 10, No. 38 (2017); and O'Sullvan, Elizabeth. “An update on PPAR activation by cannabinoids” Br J Pharmcol v. 173(12) (2016)).

In some embodiments, cannabinoids comprise endocannabinoids, which are substances produced within the body, and phytocannabinoids, which are cannabinoids that are naturally produced by plants of genus Cannabis. In some embodiments, phytocannabinoids comprise the acidic and decarboxylated acid forms of the naturally-occurring plant-derived cannabinoids, and their synthetic and biosynthetic equivalents.

Over 94 phytocannabinoids have been identified to date (Berman, Paula, et al. “A new ESI-LC/MS approach for comprehensive metabolic profiling of phytocannabinoids in Cannabis.” Scientific reports 8.1 (2018): 14280; El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Rudolf Brenneisen, 2007, Chemistry and Analysis of Phytocannabinoids, Citti, Cinzia, et al. “A novel phytocannabinoid isolated from Cannabis sativa L. with an in vivo cannabimimetic activity higher than Δ9-tetrahydrocannabinol: Δ9-Tetrahydrocannabiphorol.” Sci Rep 9 (2019): 20335, each of which is incorporated by reference in this application in its entirety). In some embodiments, cannabinoids comprise Δ⁹-tetrahydrocannabinol (THC) type (e.g., (−)-trans-delta-9-tetrahydrocannabinol or dronabinol, (+)-trans-delta-9-tetrahydrocannabinol, (−)-cis-delta-9-tetrahydrocannabinol, or (+)-cis-delta-9-tetrahydrocannabinol), cannabidiol (CBD) type, cannabigerol (CBG) type, cannabichromene (CBC) type, cannabicyclol (CBL) type, cannabinodiol (CBND) type, or cannabitriol (CBT) type cannabinoids, or any combination thereof (see, e.g., R Pertwee, ed, Handbook of Cannabis (Oxford, UK: Oxford University Press, 2014)), which is incorporated by reference in this application in its entirety). A non-limiting list of cannabinoids comprises: cannabiorcol-C1 (CBNO), CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR,10aR)-Tetrahydrocannabiorcol-C1 (Δ⁸-THCO), Cannabiorcyclol C1 (CBLO), CBG-C1 (CBGO), Cannabinol-C2 (CBN-C2), CBND-C2, Δ⁹-THC-C2, CBD-C2, CBC-C2, A-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (+)-Cannabichromevarin-C3 (CBCV), (−)-Δ⁸-trans-THC-C3 (Δ⁸-THCV), (±)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2, Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR,10aR)-Tetrahydrocannabinol-C5 (Δ⁸-THC), (f)-Cannabichromene-C5 (CBC), (−)-Cannabidiol-C5 (CBD), (±)-(1aS,3aR,8bR,8cR)-CannabicycloiC5 (CBL), Cannabicitran-C5 (CBR), (−)-Δ⁹-(6aS,10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoΔ⁷-THC), CBE-C4, Cannabigerol-C5 (CBG), Cannabitriol-C3 (CBTV), Cannabinol methyl ether-C5 (CBNM), CBNDM-C5, 8-OH—CBN-C5 (OH-CBN), OH-CBND-C5 (OH-CBND), 10-Oxo-Δ^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 5-geranyl olivetolic acid, 5-geranyl olivetolate, 8α-Hydroxy-Δ⁹-Tetrahydrocannabinol-C5 (8α-OH-Δ⁹-THC), 8β-Hydroxy-Δ⁹-Tetrahydrocannabinol-C5 (8β-OH-Δ⁹-THC), 10α-Hydroxy-Δ⁸-Tetrahydrocannabinol-C5 (10α-OH-Δ⁸-THC), 10β-Hydroxy-Δ⁸-Tetrahydrocannabinol-C5 (10β-OH-Δ⁸-THC), 10α-hydroxy-Δ^{9,11}-hexahydrocannabinol-C5, 9β,10β-Epoxyhexahydrocannabinol-C5, OH-CBD-C5 (OH-CBD), Cannabigerol monomethyl ether-C5 (CBGM), Cannabichromanone-C5, CBT-C4, (±)-6,7-cis-epoxycannabigerol-C5, (+)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (+)-cis-Cannabitriol-C5 ((t)-cis-CBT), (−)-trans-10-Ethoxy-9-hydroxy-Δ^{6a(10a)}-tetrahydrocannabivarin-C3 [(−)-trans-CBT-OEt], (−)-(6aR,9S,10S,10aR)-9,10-Dihydroxyhexahydrocannabinol-C5 [(−)-Cannabiripsol] (CBR), Cannabichromanone C-C5, (−)-6a,7,10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ9-TetrahydrocannabinolC5 (11-OAc-Δ9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C5, 4-terpenyl cannabinolate-C5, β-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylheptyl, (±)-(3S,4S)-7-hydroxy-Δ⁶-tetrahydrocannabinol-1,1-dimethylheptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl, tetrahydrocannabiphorol (THCP), cannabidiphorol (CBDP), CBGP, CBCP, their acidic forms, salts of the acidic forms, dimers of any combination of the above, trimers of any combination of the above, polymers of any combination of the above, or any combination thereof.

A cannabinoid described in this application can be a rare cannabinoid. For example, in some embodiments, a cannabinoid described in this application corresponds to a cannabinoid that is naturally produced in conventional Cannabis varieties at concentrations of less than 10%, 9%, 8%, 7%, 6%, 5%, 4%, 3%, 2%, 1%, 0.9%, 0.8%, 0.7%, 0.6%, 0.5%, 0.25%, or 0.1% by dry weight of the female flower. In some embodiments, rare cannabinoids include CBGA, CBGVA, THCVA, CBDVA, CBCVA, and CBCA. In some embodiments, rare cannabinoids are cannabinoids that are not THCA, THC, CBDA or CBD.

A cannabinoid described in this application can also be a non-rare cannabinoid.

In some embodiments, the cannabinoid is selected from the cannabinoids listed in Table 1.

TABLE 1
Non-limiting examples of cannabinoids according to the present disclosure.

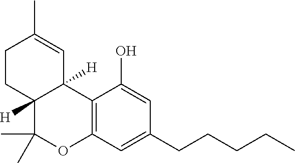 [SMILES: CCCCCc1cc(O)c2c(c1)OC(C)(C)[C@@H]1CCC(C)=C[C@@H]21]

Δ⁹-Tetrahydro-
cannabinol
Δ⁹-THC-C₅

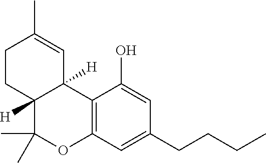 [SMILES: CCCCc1cc(O)c2c(c1)OC(C)(C)[C@@H]1CCC(C)=C[C@@H]21]

Δ⁹-Tetrahydro-
cannabinol-C₄
Δ⁹-THC-C₄

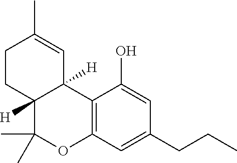 [SMILES: CCCc1cc(O)c2c(c1)OC(C)(C)[C@@H]1CCC(C)=C[C@@H]21]

Δ⁹-Tetrahydro-
cannabivarin
Δ⁹-THCV-C₃

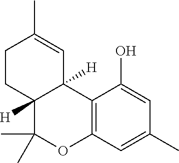

Δ⁹-Tetrahydro-
cannabiorcol
Δ⁹-THCO-C₁

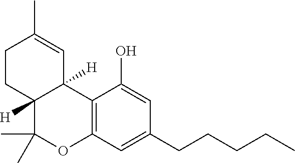 [SMILES: CCCCCc1cc(O)c2c(c1)OC(C)(C)[C@@H]1CCC(C)=C[C@@H]21]

(−)-(6aS, 10aR)-Δ⁹-
Tetrahydro-
cannabinol
(−)-cis-Δ⁹-THC-C₅

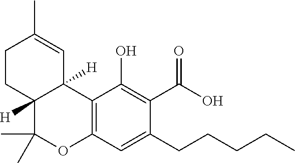 [SMILES: CCCCCc1cc2c(c(O)c1C(=O)O)[C@@H]1C=C(C)CC[C@H]1C(C)(C)O2]

Δ⁹-Tetrahydro-
cannabinolic acid A
Δ⁹-THCA-C₅ A

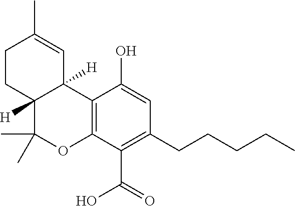 [SMILES: CCCCCc1cc(O)c2c(c1C(=O)O)OC(C)(C)[C@@H]1CCC(C)=C[C@@H]21]

Δ⁹- Tetrahydro-
cannabinolic acid B
Δ⁹-THCA-C₅ B

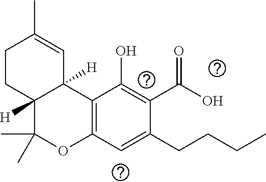 [SMILES: CCCCc1cc2c(c(O)c1C(=O)O)[C@@H]1C=C(C)CC[C@H]1C(C)(C)O2]

Δ⁹-Tetrahydro-
cannabinolic acid-C₄
A and/or B
Δ⁹-THCA-C₄ A
and/or B

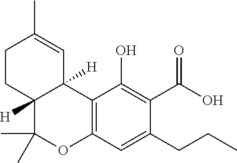 [SMILES: CCCc1cc2c(c(O)c1C(=O)O)[C@@H]1C=C(C)CC[C@H]1C(C)(C)O2]

Δ⁹-Tetrahydro-
cannabivarinic acid
A
Δ⁹-THCVA-C₃ A

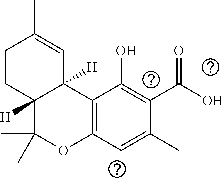 [SMILES: CC1=C[C@H]2c3c(cc(C)c(C(=O)O)c3O)OC(C)(C)[C@@H]2CC1]

Δ⁹-Tetrahydro-
cannabiorcolic acid
A and/or B
Δ⁹-THCOA-C₁ A
and/or B

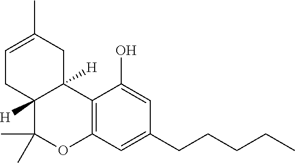 [SMILES: CCCCCc1cc(O)c2c(c1)OC(C)(C)[C@@H]1CC=C(C)C[C@@H]21]

(−)-Δ⁸-trans-
(6aR,10aR)-
Δ⁸-Tetrahydro-
cannabinol
Δ⁸-THC-C₅

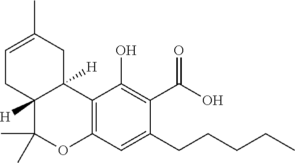 [SMILES: CCCCCc1cc2c(c(O)c1C(=O)O)[C@@H]1CC(C)=CC[C@H]1C(C)(C)O2]

(−)-Δ⁸-trans-
(6aR, 10aR)-
Tetrahydro-
cannabinolic
acid A
Δ⁸-THCA-C₅ A

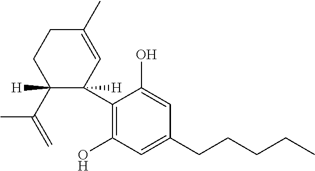 [SMILES: C=C(C)[C@@H]1CCC(C)=C[C@H]1c1c(O)cc(CCCCC)cc1O]

(−)-Cannabidiol
CBD-C5

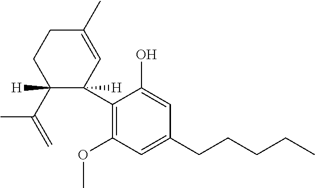 [SMILES: C=C(C)[C@@H]1CCC(C)=C[C@H]1c1c(O)cc(CCCCC)cc1OC]

Cannabidiol
momomethyl ether
CBDM-C5

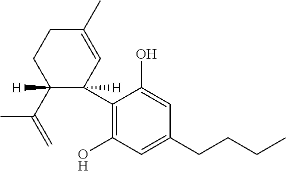 [SMILES: C=C(C)[C@@H]1CCC(C)=C[C@H]1c1c(O)cc(CCCC)cc1O]

Cannabidiol-C₄
CBD-C4

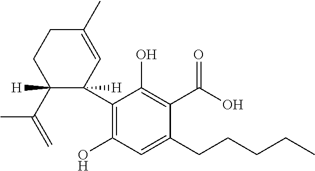 [SMILES: C=C(C)[C@@H]1CCC(C)=C[C@H]1c1c(O)cc(CCCCC)c(C(=O)O)c1O]

Cannabidiolic acid
CBDA-C5

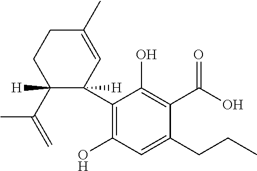 [SMILES: C=C(C)[C@@H]1CCC(C)=C[C@H]1c1c(O)cc(CCC)c(C(=O)O)c1O]

Cannabidivarinic acid
CBDVA-C3

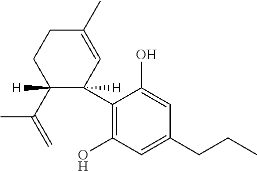 [SMILES: C=C(C)[C@@H]1CCC(C)=C[C@H]1c1c(O)cc(CCC)cc1O]

(−)-Cannabidivarin
CBDV-C3

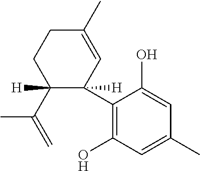 [SMILES: C=C(C)[C@@H]1CCC(C)=C[C@H]1c1c(O)cc(C)cc1O]

Cannabidiorcol
CBD-C1

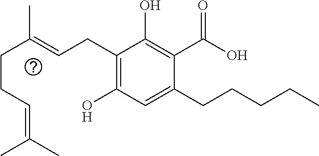 [SMILES: CCCCCc1cc(O)c(C/C=C(\C)CCC=C(C)C)c(O)c1C(=O)O]

Cannabigerolic acid
A
(E)-CBGA-C₅ A

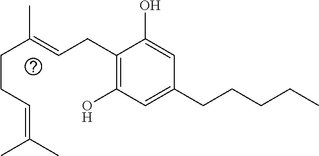 [SMILES: CCCCCc1cc(O)c(C/C=C(\C)CCC=C(C)C)c(O)c1]

Cannabigerol
(E)-CBG-C₅

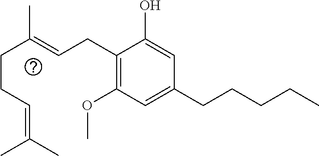 [SMILES: CCCCCc1cc(O)c(C/C=C(\C)CCC=C(C)C)c(OC)c1]

Cannabigerol
monomethyl ether
(E)-CBGM-C₅ A

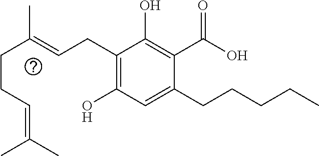 [SMILES: CCCCCc1cc(O)c(C/C=C(\C)CCC=C(C)C)c(O)c1C(=O)O]

Cannabinerolic acid
A
(Z)-CBGA-C₅ A

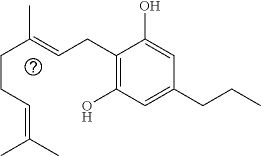 [SMILES: CCCc1cc(O)c(C/C=C(\C)CCC=C(C)C)c(O)c1]

Cannabigerovarin
(E)-CBGV-C₃

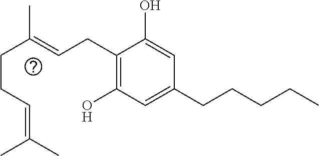 [SMILES: CCCCCc1cc(O)c(C/C=C(\C)CCC=C(C)C)c(O)c1]

Cannabigerol
(E)-CBG-C₅

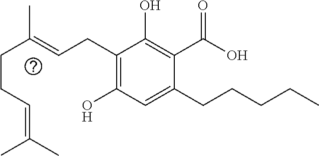 [SMILES: CCCCCc1cc(O)c(C/C=C(\C)CCC=C(C)C)c(O)c1C(=O)O]

Cannabigerolic acid
A
(E)-CBGA-C₅ A

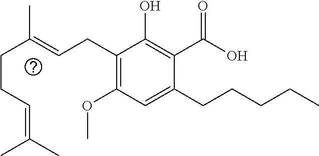 [SMILES: CCCCCc1cc(OC)c(C/C=C(\C)CCC=C(C)C)c(O)c1C(=O)O]

Cannabigerolic acid A
monomethyl ether
(E)-CBGAM-C₅ A

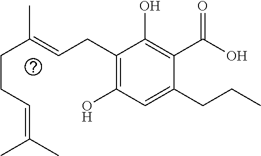 [SMILES: CCCc1cc(O)c(C/C=C(\C)CCC=C(C)C)c(O)c1C(=O)O]

Cannabigerovarinic
acid A
(E)-CBGVA-C₃ A

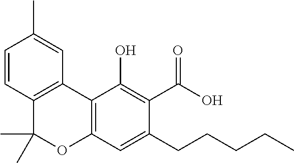 [SMILES: CCCCCc1cc2c(c(O)c1C(=O)O)-c1cc(C)ccc1C(C)(C)O2]

Cannabinolic acid A
CBNA-C5 A

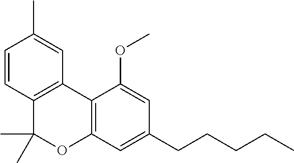 [SMILES: CCCCCc1cc(OC)c2c(c1)OC(C)(C)c1ccc(C)cc1-2]

Cannabinol methyl
ether
CBNM-C5

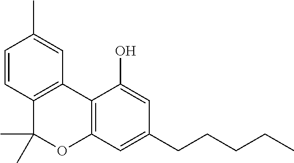 [SMILES: CCCCCc1cc(O)c2c(c1)OC(C)(C)c1ccc(C)cc1-2]

Cannabinol
CBN-C5

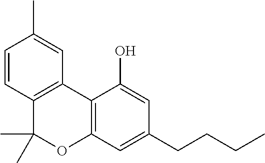 [SMILES: CCCCc1cc(O)c2c(c1)OC(C)(C)c1ccc(C)cc1-2]

Cannabinol-C4
CBN-C4

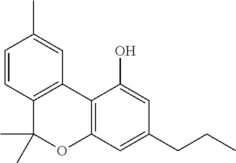 [SMILES: CCCc1cc(O)c2c(c1)OC(C)(C)c1ccc(C)cc1-2]

Cannabivarin
CBN-C3

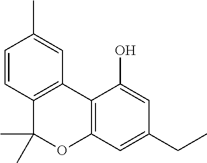 [SMILES: CCc1cc(O)c2c(c1)OC(C)(C)c1ccc(C)cc1-2]

Cannabinol-C2
CBN-C2

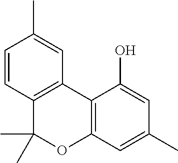

Cannabiorcol
CBN-C1

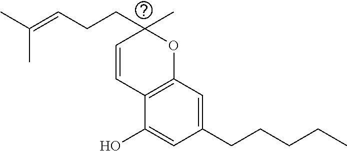 [SMILES: CCCCCc1cc(O)c2c(c1)OC(C)(CCC=C(C)C)C=C2]

(±)-Cannabichromene
CBC-C₅

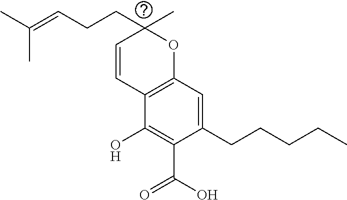 [SMILES: CCCCCc1cc2c(c(O)c1C(=O)O)C=CC(C)(CCC=C(C)C)O2]

(±)-Cannabichromenic
acid A
CBCA-C₅ A

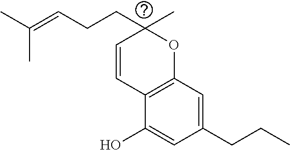 [SMILES: CCCc1cc(O)c2c(c1)OC(C)(CCC=C(C)C)C=C2]

(±)-Cannabivarichro-
mene,
(±)-
Cannabichromevarin
CBCV-C₃

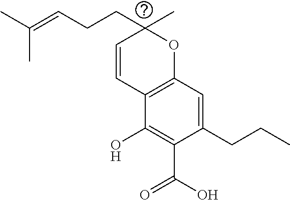 [SMILES: CCCc1cc2c(c(O)c1C(=O)O)C=CC(C)(CCC=C(C)C)O2]

(±)-Cannabichro-
mevarinic
acid A
CBCVA-C₃ A

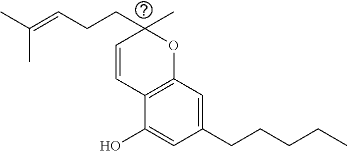 [SMILES: CCCCCc1cc(O)c2c(c1)OC(C)(CCC=C(C)C)C=C2]

(±)-
Cannabichromene
CBC-C₅

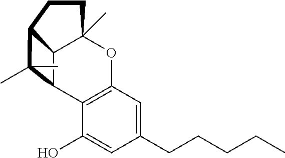

(±)-
(1aS,3aR,8bR,8cR)-
Cannabicyclol
CBL-C₅

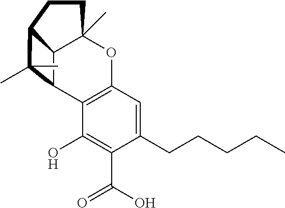

(±)-
(1aS,3aR,8bR,8cR)-
Cannabicyclolic acid
A
CBLA-Cs A

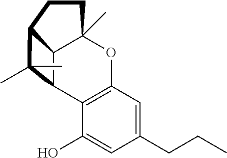

(±)-
(1aS,3aR,8bR,8cR)-
Cannabicyclovarin
CBLV-C₃

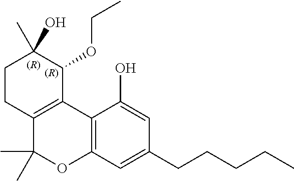

(−)-(9R,10R)-trans-
10-O-Ethyl-
cannabitriol
(−)-trans-CBT-OEt-
C5

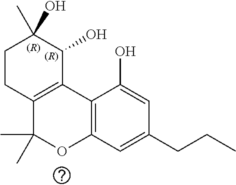

(±)-
(9R,10R/98,10S)-
Cannabitriol-C3
(±)-trans-CBT-C3

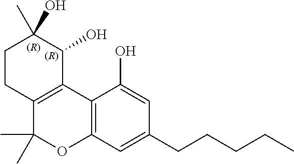

(−)-(9R,10R)-trans-
Cannabitriol
(−)-trans-CBT-C5

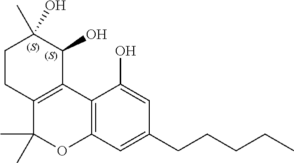

(+)-(9S,10S)-
Cannabitriol
(+)-trans-CBT-C5

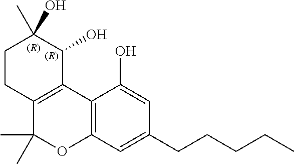

(±)-
(9R,108/9S,10R)-
Cannabitriol
(+)-cis-CBT-C5

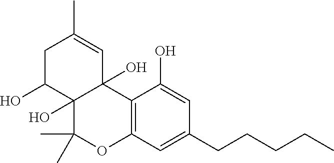 [SMILES: CCCCCc1cc(O)c2c(c1)OC(C)(C)C1(O)C(O)CC(C)=CC21O]

(−)-6a,7,10a-
Trihydroxy-
Δ9-tetrahydro-
cannabinol
(−)-Cannabitetrol

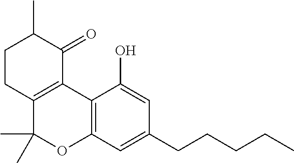 [SMILES: CCCCCc1cc(O)c2c(c1)OC(C)(C)C1=C2C(=O)C(C)CC1]

10-Oxo-A6a(10a)-
tetrahydro-
cannabinol
OTHC

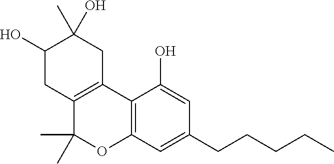 [SMILES: CCCCCc1cc(O)c2c(c1)OC(C)(C)C1=C2CC(C)(O)C(O)C1]

8,9-Dihydroxy-
Δ6a(10a)-
tetrahydro-cannabinol
8,9-Di-OH-CBT-C5

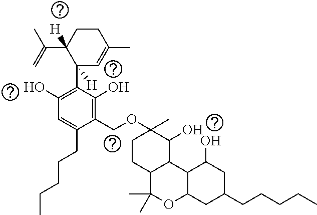 [SMILES: C=C(C)[C@@H]1CCC(C)=C[C@H]1c1c(O)cc(CCCCC)c(COC2(C)CCC3C(C4C(O)CC(CCCCC)CC4OC3(C)C)C2O)c1O]

Cannabidiolic acid A
cannabitriol ester
CBDA-C5 9-OH-
CBT-C5 ester

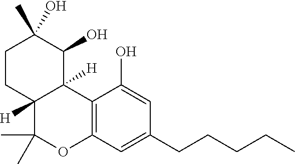 [SMILES: CCCCCc1cc(O)c2c(c1)OC(C)(C)[C@@H]1CC[C@](C)(O)[C@@H](O)[C@@H]21]

(−)-
(6aR,9S,10S,10aR)-
9,10-Dihydroxy-
bexahydro-
cannabinol,
Cannabiripsol
Cannabiripsol-C5

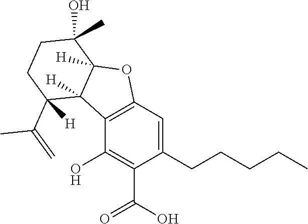

(5aS,6S,9R,9aR)-
Cannabielsoic acid B
CBEA-C5 B

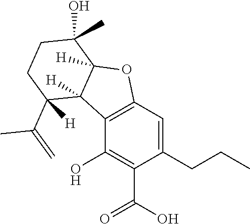

(5aS,68,9R,9aR)-
C3-Cannabielsoic
acid B
CBEA-C3 B

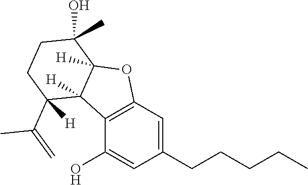

(5aS,6S,9R,9aR)-
Cannabielsoin
CBE-C5

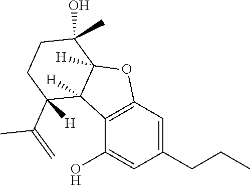

(5aS,6S,9R,9aR)-
C3-Cannabielsoin
CBE-C3

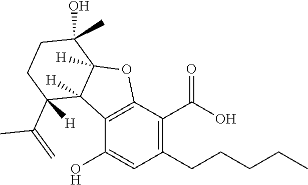

(5aS,6S,9R,9aR)-
Cannabielsoic acid A
CBEA-C5 A

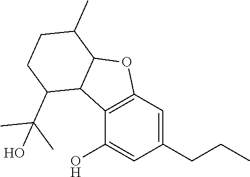 [SMILES: CCCc1cc(O)c2c(c1)OC1C(C)CCC(C(C)(C)O)C21]

Cannabiglendol-C3
OH-iso-HHCV-C3

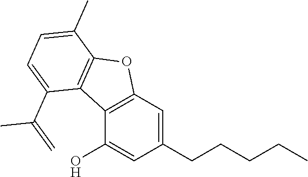 [SMILES: C=C(C)c1ccc(C)c2oc3cc(CCCCC)cc(O)c3c12]

Dehydro-
cannabifuran
DCBF-C5

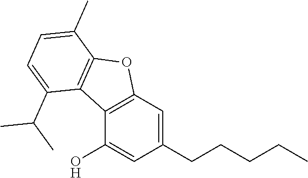 [SMILES: CCCCCc1cc(O)c2c(c1)oc1c(C)ccc(C(C)C)c12]

Cannabifuran
CBF-C5

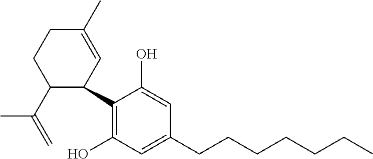 [SMILES: C=C(C)C1CCC(C)=C[C@H]1c1c(O)cc(CCCCCCC)cc1O]

Cannabidiphorol
(CBDP)

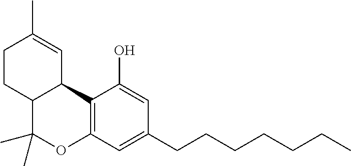

Tetrahydro-
cannabiphorol
(THCP)

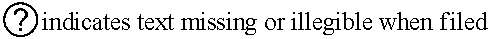

Cannabinoids are often classified by “type,” i.e., by the topological arrangement of their prenyl moieties (See, for example, M. A. Elsohly and D. Slade, Life Sci., 2005, 78, 539-548; and L. O. Hanus et al. Nat. Prod. Rep., 2016, 33, 1357). Generally, each “type” of cannabinoid includes the variations possible for ring substitutions of the resorcinol moiety at the position meta to the two hydroxyl moieties. As used herein, a “CBG-type” cannabinoid is a 3-[(2E)-3,7-dimethylocta-2,6-dienyl]-2,4-dihydroxybenzoic acid optionally substituted at the 6 position of the benzoic acid moiety. As used herein, “CBG-type” cannabinoids refer to 5-hydroxy-2-methyl-2-(4-methylpent-3-enyl)-chromene-6-carboxylic acid optionally substituted at the 7 position of the chromene moiety. As used herein, a “THC-type” cannabinoid is a (6aR,10aR)-1-hydroxy-6,6,9-trimethyl-6a,7, 8,10a-tetrahydrobenzo[c]chromene-2-carboxylic acid optionally substituted at the 3 position of the benzo[c]chromene moiety. As used herein, a “CBD-type” cannabinoid is a 2,4-dihydroxy-3-[(1R,6R)-3-methyl-6-prop-1-en-2-ylcyclohex-2-en-1-yl]-benzoic acid optionally substituted at the 6 position of the benzoic acid moiety. In some embodiments, the optional ring substitution for each “type” is an optionally substituted C1-C11 alkyl, an optionally substituted C1-C11 alkenyl, an optionally substituted C1-C11 alkynyl, or an optionally substituted C1-C11 aralkyl.

### Biosynthesis of Cannabinoids and Cannabinoid Precursors

Aspects of the present disclosure provide tools, sequences, and methods for the biosynthetic production of cannabinoids in host cells. In some embodiments, the present disclosure teaches expression of enzymes that are capable of producing cannabinoids by biosynthesis.

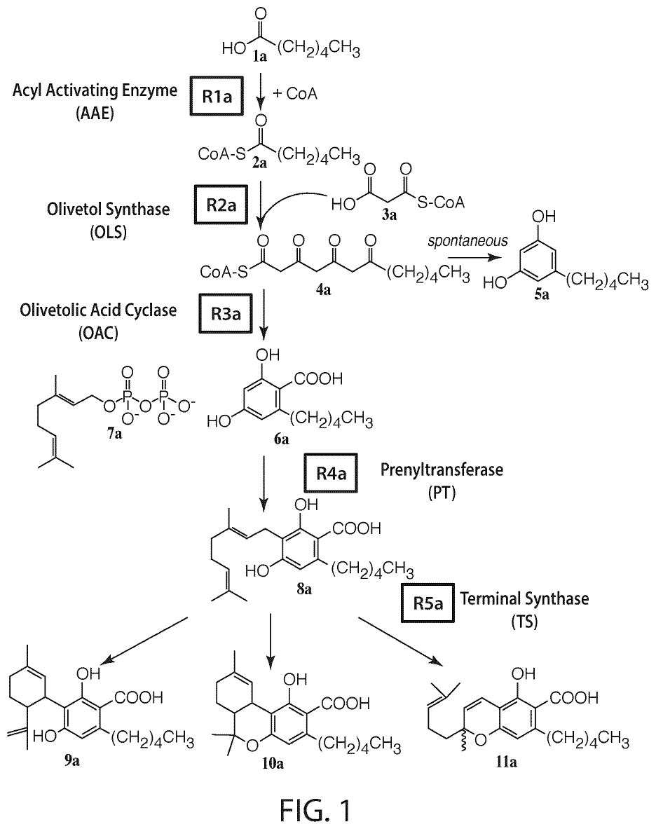

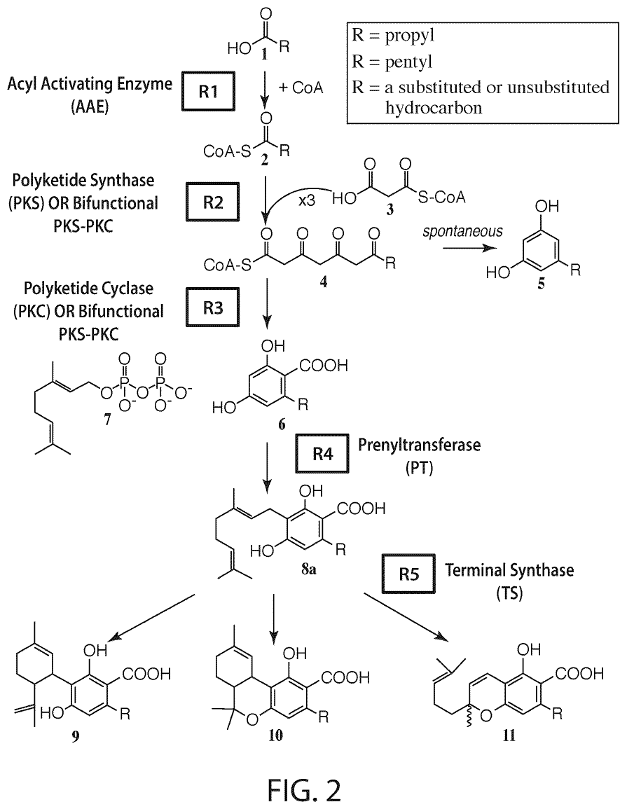

As a non-limiting example, one or more of the enzymes depicted in FIG. 2 may be used to produce a cannabinoid or cannabinoid precursor of interest. FIG. 1 shows a cannabinoid biosynthesis pathway for the most abundant phytocannabinoids found in Cannabis. See also, de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), and Carvalho et al. “Designing Microorganisms for Heterologous Biosynthesis of Cannabinoids” (2017) FEMS Yeast Research June 1; 17(4), each of which is incorporated by reference in this application in its entirety.

It should be appreciated that a precursor substrate for use in cannabinoid biosynthesis is generally selected based on the cannabinoid of interest. Non-limiting examples of cannabinoid precursors include compounds of Formulae (1)-(8) in FIG. 2. In some embodiments, polyketides, including compounds of Formula (5), could be prenylated. In certain embodiments, the precursor is a precursor compound shown in FIG. 1, 2, or 3. Substrates in which R contains 1-40 carbon atoms are preferred. In some embodiments, substrates in which R contains 3-8 carbon atoms are most preferred.

As used in this application, a cannabinoid or a cannabinoid precursor may comprise an R group. See, e.g., FIG. 2. In some embodiments, R may be a hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C10 alkyl. In certain embodiments, R is optionally substituted C1-C8 alkyl. In certain embodiments, R is optionally substituted C1-C5 alkyl. In certain embodiments, R is optionally substituted C1-C7 alkyl. In certain embodiments, R is optionally substituted C3-C5 alkyl. In certain embodiments, R is optionally substituted C3 alkyl. In certain embodiments, R is unsubstituted C3 alkyl. In certain embodiments, R is n-C3 alkyl. In certain embodiments, R is n-propyl. In certain embodiments, R is n-butyl. In certain embodiments, R is n-pentyl. In certain embodiments, R is n-hexyl. In certain embodiments, R is n-heptyl. In certain embodiments, R is of formula:

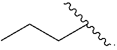 [SMILES: *CCC]

In certain embodiments, R is optionally substituted C4 alkyl. In certain embodiments, R is unsubstituted C4 alkyl. In certain embodiments, R is optionally substituted C5 alkyl. In certain embodiments, R is unsubstituted C5 alkyl. In certain embodiments, R is optionally substituted C6 alkyl. In certain embodiments, R is unsubstituted C6 alkyl. In certain embodiments, R is optionally substituted C7 alkyl. In certain embodiments, R is unsubstituted C7 alkyl. In certain embodiments, R is of formula:

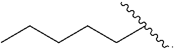 [SMILES: *CCCCC]

In certain embodiments, R is of formula:

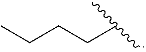 [SMILES: *CCCC]

In certain embodiments, R is of formula:

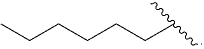 [SMILES: *CCCCCC]

In certain embodiments, R is of formula:

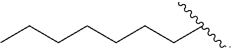 [SMILES: *CCCCCCC]

In certain embodiments, R is of formula:

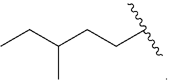 [SMILES: *CCC(C)CC]

In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

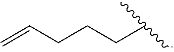 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

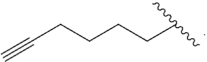 [SMILES: *CCCCC#C]

In certain embodiments, R is optionally substituted carbocyclyl. In certain embodiments, R is optionally substituted aryl (e.g., phenyl or napthyl).

The chain length of a precursor substrate can be from C1-C40. Those substrates can have any degree and any kind of branching or saturation or chain structure, including, without limitation, aliphatic, alicyclic, and aromatic. In addition, they may include any functional groups including hydroxy, halogens, carbohydrates, phosphates, methyl-containing or nitrogen-containing functional groups.

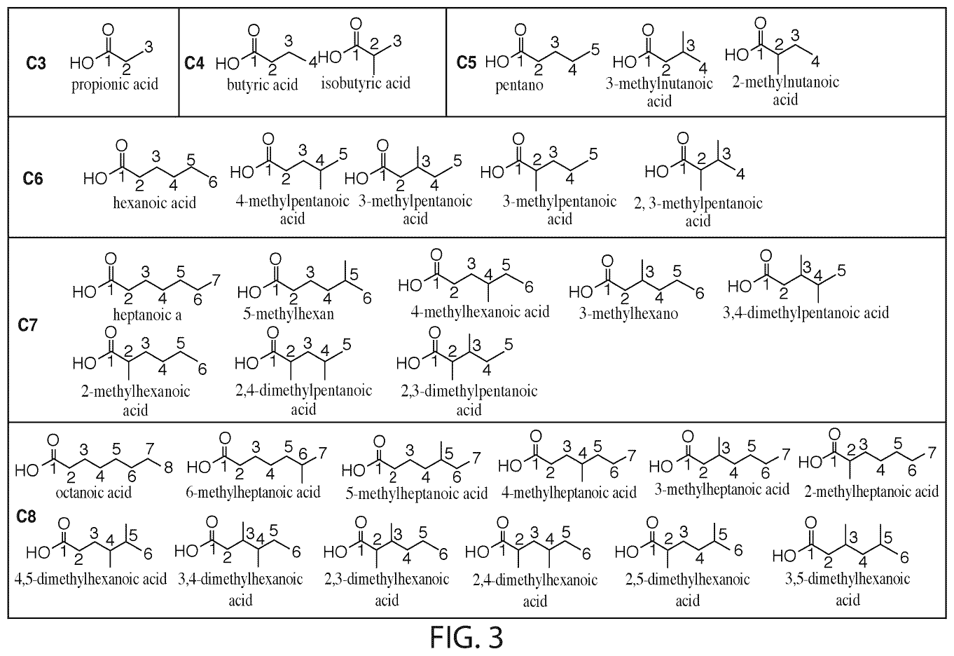

For example, FIG. 3 shows a non-exclusive set of putative precursors for the cannabinoid pathway. Aliphatic carboxylic acids including four to eight total carbons (“C4”-“C8” in FIG. 3) and up to 10-12 total carbons with either linear or branched chains may be used as precursors for the heterologous pathway. Non-limiting examples include methanoic acid, butyric acid, pentanoic acid, hexanoic acid, heptanoic acid, isovaleric acid, octanoic acid, and decanoic acid. Additional precursors may include ethanoic acid and propanoic acid. In some embodiments, in addition to acids, the ester, salt, and acid forms may all be used as substrates. Substrates may have any degree and any kind of branching, saturation, and chain structure, including, without limitation, aliphatic, alicyclic, and aromatic. In addition, they may include any functional modifications or combination of modifications including, without limitation, halogenation, hydroxylation, amination, acylation, alkylation, phenylation, and/or installation of pendant carbohydrates, phosphates, sulfates, heterocycles, or lipids, or any other functional groups.

Substrates for any of the enzymes disclosed in this application may be provided exogenously or may be produced endogenously by a host cell. In some embodiments, the cannabinoids are produced from a glucose substrate, so that compounds of Formula 1 shown in FIG. 2 and CoA precursors are synthesized by the cell. In other embodiments, a precursor is fed into the reaction. In some embodiments, a precursor is a compound selected from Formulae 1-8 in FIG. 2.

Cannabinoids produced by methods disclosed in this application include rare cannabinoids. Due to the low concentrations at which cannabinoids, including rare cannabinoids occur in nature, producing industrially significant amounts of isolated or purified cannabinoids from the Cannabis plant may become prohibitive due to, e.g., the large volumes of Cannabis plants, and the large amounts of space, labor, time, and capital requirements to grow, harvest, and/or process the plant materials (see, for example, Crandall, K., 2016. A Chronic Problem: Taming Energy Costs and Impacts from Marijuana Cultivation. EQ Research; Mills, E., 2012. The carbon footprint of indoor Cannabis production. Energy Policy, 46, pp. 58-67; Jourabchi, M. and M. Lahet. 2014. Electrical Load Impacts of Indoor Commercial Cannabis Production. Presented to the Northwest Power and Conservation Council; O'Hare, M., D. Sanchez, and P. Alstone. 2013. Environmental Risks and Opportunities in Cannabis Cultivation. Washington State Liquor and Cannabis Board; 2018. Comparing Cannabis Cultivation Energy Consumption. New Frontier Data; and Madhusoodanan, J., 2019. Can cannabis go green? Nature Outlook: Cannabis; all of which are incorporated by reference in this disclosure). The disclosure provided in this application represents a potentially efficient method for producing high yields of cannabinoids, including rare cannabinoids. The disclosure provided in this application also represents a potential method for addressing concerns related to agricultural practices and water usage associated with traditional methods of cannabinoid production (Dillis et al. “Water storage and irrigation practices for cannabis drive seasonal patterns of water extraction and use in Northern California.” Journal of Environmental Management 272 (2020): 110955, incorporated by reference in this disclosure).

Cannabinoids produced by the disclosed methods also include non-rare cannabinoids. Without being bound by a particular theory, the methods described in this application may be advantageous compared with traditional plant-based methods for producing non-rare cannabinoids. For example, methods provided in this application represent potentially efficient means for producing consistent and high yields of non-rare cannabinoids. With traditional methods of cannabinoid production, in which cannabinoids are harvested from plants, maintaining consistent and uniform conditions, including airflow, nutrients, lighting, temperature, and humidity, can be difficult. For example, with plant-based methods, there can be microclimates created by branching, which can lead to inconsistent yields and by-product formation. In some embodiments, the methods described in this application are more efficient at producing a cannabinoid of interest as compared to harvesting cannabinoids from plants. For example, with plant-based methods, seed-to-harvest can take up to half a year, while cutting-to-harvest usually takes about 4 months. Additional steps including drying, curing, and extraction are also usually needed with plant-based methods. In contrast, in some embodiments, the fermentation-based methods described in this application only take about 1, 2, 3, 4, 5, 6, 7, 8, 9, or 10 days. In some embodiments, the fermentation-based methods described in this application only take about 3-5 days. In some embodiments, the fermentation-based methods described in this application only take about 5 days. In some embodiments, the methods provided in this application reduce the amount of security needed to comply with regulatory standards. For example, a smaller secured area may be needed to be monitored and secured to practice the methods described in this application as compared to the cultivation of plants. In some embodiments, the methods described in this application are advantageous over plant-sourced cannabinoids.

### Terminal Synthases (TS)

A host cell described in this application may comprise a terminal synthase (TS). As used in this application, a “TS” refers to an enzyme that is capable of catalyzing oxidative cyclization of a prenyl moiety (e.g., terpene) to produce a ring-containing product (e.g., heterocyclic ring-containing product). In certain embodiments, a TS is capable of catalyzing oxidative cyclization of a prenyl moiety (e.g., terpene) to produce a carbocyclic-ring containing product (e.g., cannabinoid). In certain embodiments, a TS is capable of catalyzing oxidative cyclization of a prenyl moiety (e.g., terpene) to produce a heterocyclic-ring containing product (e.g., cannabinoid). In certain embodiments, a TS is capable of catalyzing oxidative cyclization of a prenyl moiety (e.g., terpene) to produce a cannabinoid.

TS enzymes are monomers that include FAD-binding and Berberine Bridge Enzyme (BBE) sequence motifs.

In some embodiments, the TS is an “ancestral” terminal synthase. Ancestral TSes can be generated from probabilistic models of mutations applied to terminal synthase phylogenes based on transcriptomic datasets. For example, Hochberg et al., describe a process for reconstructing ancestral proteins in Annu. Rev. Biophys. 2017. 46:247-69, which is incorporated by reference in its entirety in this disclosure.

a. Substrates

A TS may be capable of using one or more substrates. In some instances, the location of the prenyl group and/or the R group differs between TS substrates. For example, a TS may be capable of using as a substrate one or more compounds of Formula (8w), Formula (8x), Formula (8′), Formula (8y), and/or Formula (8z):

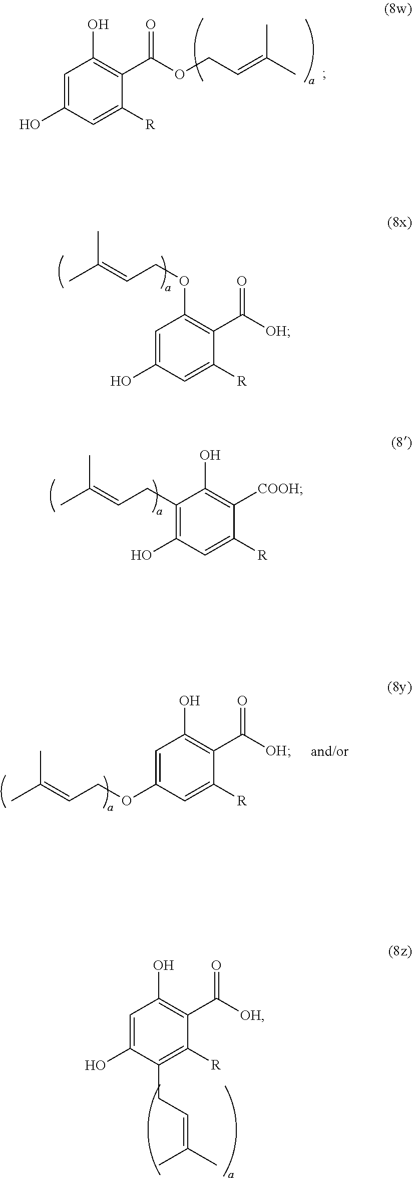

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

In certain embodiments, a compound of Formula (8′) is a compound of Formula (8):

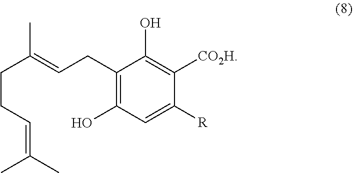

In some embodiments, R is hydrogen, an optionally substituted C1-C11 alkyl, an optionally substituted C1-C11 alkenyl, an optionally substituted C1-C11 alkynyl, or an optionally substituted C1-C11 aralkyl.

In some embodiments, a TS catalyzes oxidative cyclization of the prenyl moiety (e.g., terpene) of a compound of Formula (8) described in this application and shown in FIG. 2. In certain embodiments, a compound of Formula (8) is a compound of Formula (8a):

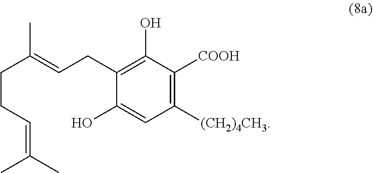

In some embodiments, the production of a compound of Formula (11) from a particular substrate may be assessed relative to the production of a compound of Formula (11) from a control substrate. In some embodiments, the production of a compound of Formula (10) from a particular substrate may be assessed relative to the production of a compound of Formula (10) from a control substrate. In some embodiments, the production of a compound of Formula (9) from a particular substrate may be assessed relative to the production of a compound of Formula (9) from a control substrate.

b. Products

In some embodiments, TS enzymes catalyze the formation of CBD-type cannabinoids, THC-type cannabinoids and/or CBC-type cannabinoids from CBG-type cannabinoids. In embodiments where CBGA is the substrate, the TS enzymes CBDAS, THCAS and CBCAS would generally catalyze the formation of cannabidiolic acid (CBDA), A9-tetrahydrocannabinolic acid (THCA) and cannabichromenic acid (CBCA), respectively. However, in some embodiments, a TS can produce more than one different product depending on reaction conditions. Product promiscuity has been noted among the Cannabis terminal synthases (e.g., Zirpel et al., J. Biotechnol. 2018 Apr. 20, 272:40-7). Without wishing to be bound by any theory, it is believed that the reaction conditions affect the protonation state and orientation of the amino acids that form the substrate binding site of the TS enzymes, which may affect the docking of the substrate and/or products of these enzymes. For example, the pH of the reaction environment may cause a THCAS or a CBDAS to produce CBCA in greater proportions than THCA or CBDAS, respectively (see, for example, U.S. Pat. No. 9,359,625 to Winnicki and Donsky, incorporated by reference in its entirety). In some embodiments, a TS has a predetermined product specificity in intracellular conditions, such as cytosolic conditions or organelle conditions. By expressing a TS with a predetermined product specificity based on intracellular conditions, in vivo products produced by a cell expressing the TS may be more predictably produced. In some embodiments, a TS produces a desired product at a pH of 5.5. In some embodiments, a TS produces a desired product at a pH of 1, 2, 3, 4, 5, 6, 7, 8, 9, 10, 11, 12, 13 or 14. In some embodiments, a TS produces a desired product at a pH that is between 4.5 and 8.0. In some embodiments, a TS produces a desired product at a pH that is between 5 and 6. In some embodiments, a TS produces a desired product at a pH that is around 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, including all values in between. In some embodiments, the product profile of a TS is dependent on the TS's signal peptide because the signal peptide targets the TS to a particular intracellular location having particular intracellular conditions (e.g., a particular organelle) that regulate the type of product produced by the TS. Exemplary signal peptides are discussed in further detail below. Differences in the intracellular conditions can affect the activity of the TS enzymes, for example, due to variations in pH and/or differences in the folding of TS enzymes due to the presence of chaperone proteins.

A TS may be capable of using one or more substrates described in this application to produce one or more products. Non-limiting example of TS products are shown in Table 1. In some instances, a TS is capable of using one substrate to produce 1, 2, 3, 4, 5, 6, 7, 8, 9, or 10 different products. In some embodiments, a TS is capable of using more than one substrate to produce 1, 2, 3, 4, 5, 6, 7, 8, 9, or 10 different products.

In some embodiments, a TS is capable of producing a compound of Formula (X-A) and/or a compound of Formula (X-B):

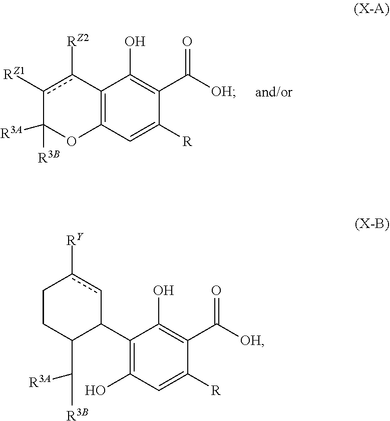

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;
R is hydrogen, optionally substituted acyl, optionally substituted alkyl, optionally substituted alkenyl, optionally substituted alkynyl, optionally substituted carbocyclyl, or optionally substituted aryl;
R^{Z1} is hydrogen, optionally substituted acyl, optionally substituted alkyl, optionally substituted alkenyl, optionally substituted alkynyl, optionally substituted carbocyclyl, or optionally substituted aryl;
R^{Z2} is hydrogen, optionally substituted acyl, optionally substituted alkyl, optionally substituted alkenyl, optionally substituted alkynyl, optionally substituted carbocyclyl, or optionally substituted aryl;
or optionally, R^{Z1} and R^{Z2} are taken together with their intervening atoms to form an optionally substituted carbocyclic ring;
R^{3A} is hydrogen, optionally substituted acyl, optionally substituted alkyl, optionally substituted alkenyl, or optionally substituted alkynyl;
R^{3B} is hydrogen, optionally substituted acyl, optionally substituted alkyl, optionally substituted alkenyl, or optionally substituted alkynyl, and/or
R^{Y} is hydrogen, optionally substituted acyl, optionally substituted alkyl, optionally substituted alkenyl, or optionally substituted alkynyl.

In some embodiments, a compound of Formula (X-A) is:

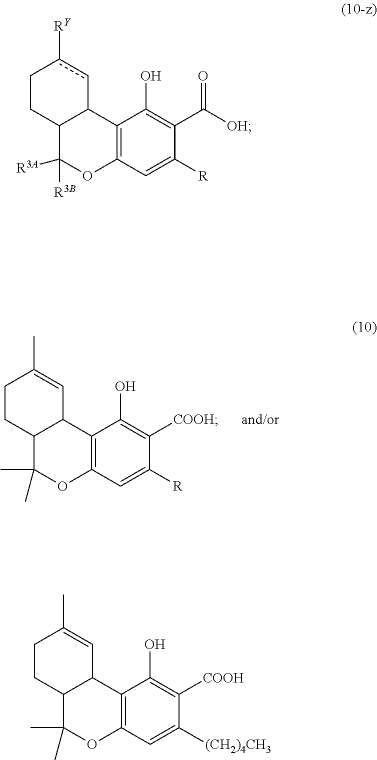

(Tetrahydrocannabinolic acid (THCA) (10a)).

In certain embodiments, a compound of Formula (10)

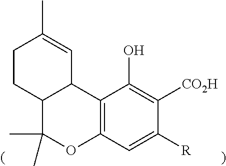

has a chiral atom labeled with * at carbon 10 and a chiral atom labeled with ** at carbon 6. In certain embodiments, in a compound of Formula (10)

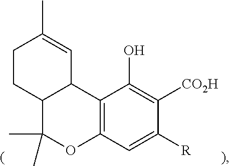

the chiral atom labeled with * at carbon 10 is of the R-configuration or S-configuration; and a chiral atom labeled with ** at carbon 6 is of the R-configuration. In certain embodiments, in a compound of Formula (10)

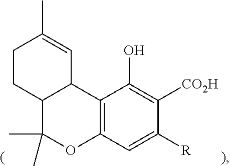

the chiral atom labeled with * at carbon 10 is of the S-configuration; and a chiral atom labeled with ** at carbon 6 is of the R-configuration or S-configuration. In certain embodiments, in a compound of Formula (10)

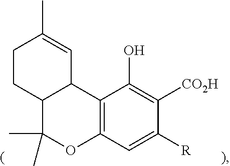

the chiral atom labeled with * at carbon 10 is of the R-configuration and a chiral atom labeled with ** at carbon 6 is of the R-configuration. In certain embodiments, a compound of Formula (10)

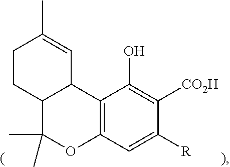

is of the formula:

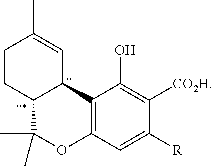

In certain embodiments, in a compound of Formula (10)

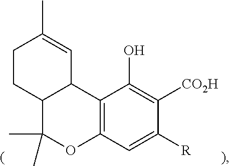

the chiral atom labeled with * at carbon 10 is of the S-configuration and a chiral atom labeled with ** at carbon 6 is of the S-configuration. In certain embodiments, a compound of Formula (10)

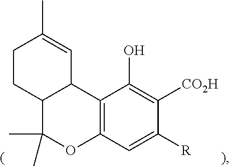

is of the formula:

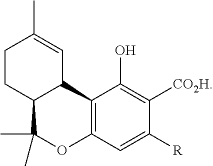

In certain embodiments, a compound of Formula (10a)

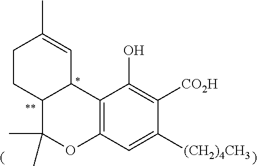

has a chiral atom labeled with * at carbon 10 and a chiral atom labeled with ** at carbon 6. In certain embodiments, in a compound of Formula (10a)

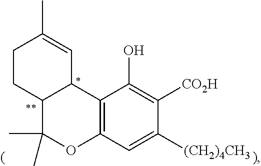

the chiral atom labeled with * at carbon 10 is of the R-configuration or S-configuration; and a chiral atom labeled with ** at carbon 6 is of the R-configuration. In certain embodiments, in a compound of Formula (10a)

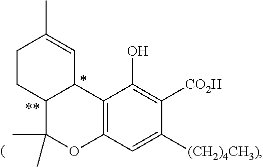

the chiral atom labeled with * at carbon 10 is of the S-configuration; and a chiral atom labeled with ** at carbon 6 is of the R-configuration or S-configuration. In certain embodiments, in a compound of Formula (10a)

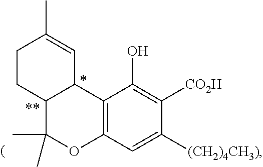

the chiral atom labeled with * at carbon 10 is of the R-configuration and a chiral atom labeled with ** at carbon 6 is of the R-configuration. In certain embodiments, a compound of Formula (10a)

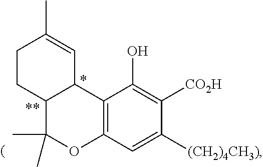

is of the formula

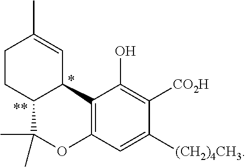

In certain embodiments, in a compound of Formula (10a)

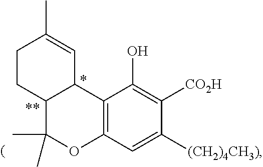

the chiral atom labeled with * at carbon 10 is of the S-configuration and a chiral atom labeled with ** at carbon 6 is of the S-configuration. In certain embodiments, a compound of Formula (10a)

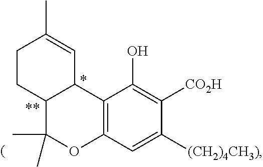

is of the formula:

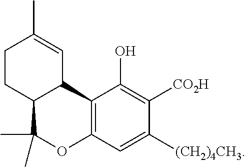

In some embodiments, a compound of Formula (X-A) is:

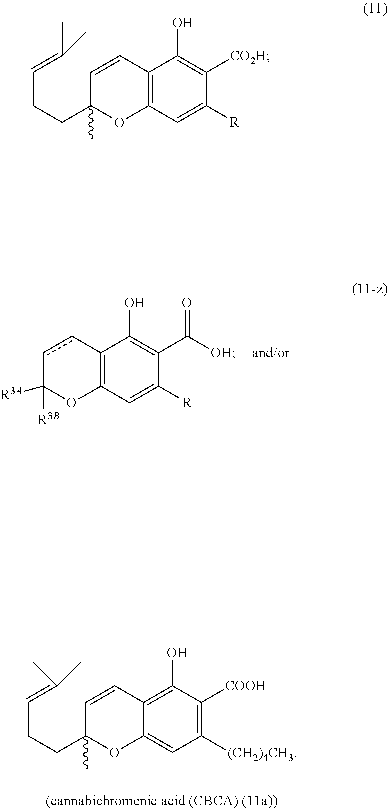

In some embodiments, a compound of Formula (X-A) is;

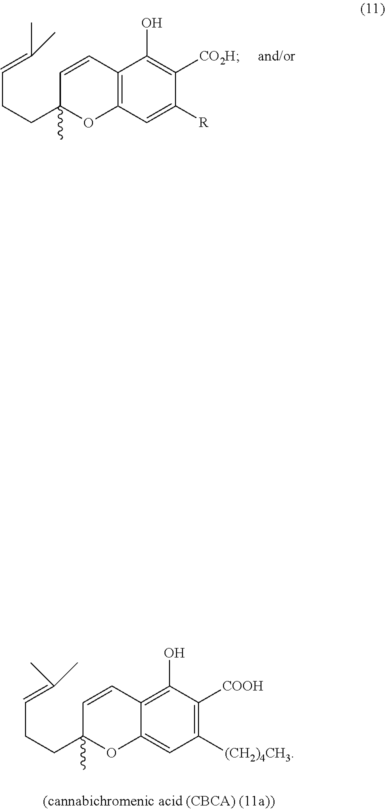

In some embodiments, a compound of Formula (X-B) is:

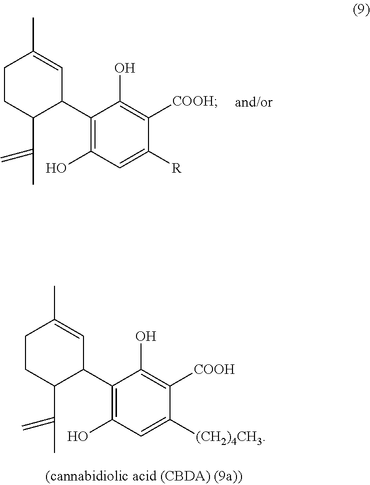

In certain embodiments, a compound of Formula (9)

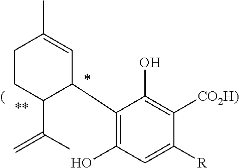

has a chiral atom labeled with * at carbon 3 and a chiral atom labeled with ** at carbon 4. In certain embodiments, in a compound of Formula (9)

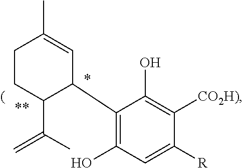

the chiral atom labeled with * at carbon 3 is of the R-configuration or S-configuration; and a chiral atom labeled with ** at carbon 4 is of the R-configuration. In certain embodiments, in a compound of Formula (9)

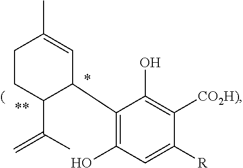

the chiral atom labeled with * at carbon 3 is of the 5-configuration; and a chiral atom labeled with ** at carbon 4 is of the R-configuration or S-configuration. In certain embodiments, in a compound of Formula (9)

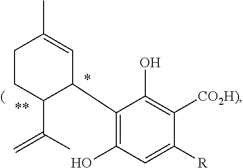

the chiral atom labeled with * at carbon 3 is of the R-configuration and a chiral atom labeled with ** at carbon 4 is of the R-configuration. In certain embodiments, a compound of Formula (9)

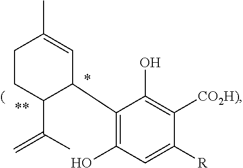

is of the formula:

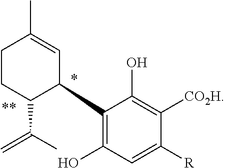

In certain embodiments, in a compound of Formula (9)

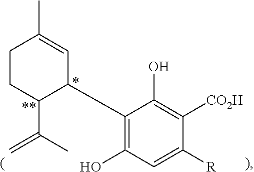

the chiral atom labeled with * at carbon 3 is of the S-configuration and a chiral atom labeled with ** at carbon 4 is of the S-configuration. In certain embodiments, a compound of Formula (9)

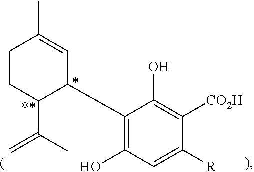

is of the formula:

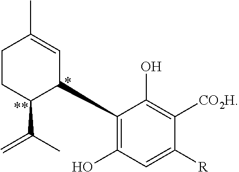

In certain embodiments, a compound of Formula (9a) (CBDA)

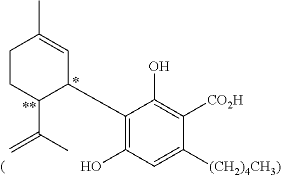

has a chiral atom labeled with * at carbon 3 and a chiral atom labeled with ** at carbon 4. In certain embodiments, in a compound of Formula (9a)

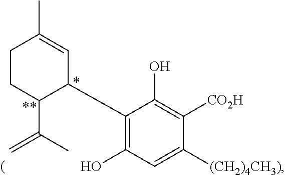

the chiral atom labeled with * at carbon 3 is of the R-configuration or S-configuration; and a chiral atom labeled with ** at carbon 4 is of the R-configuration. In certain embodiments, in a compound of Formula (9a)

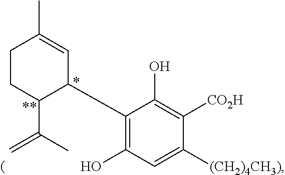

the chiral atom labeled with * at carbon 3 is of the S-configuration; and a chiral atom labeled with ** at carbon 4 is of the R-configuration or S-configuration. In certain embodiments, in a compound of Formula (9a)

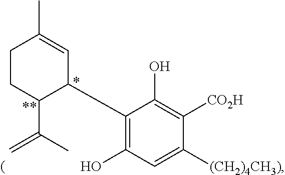

the chiral atom labeled with * at carbon 3 is of the R-configuration and a chiral atom labeled with ** at carbon 4 is of the R-configuration. In certain embodiments, a compound of Formula (9a)

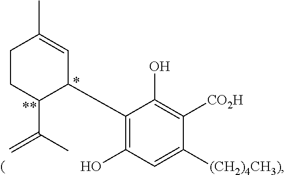

is of the formula:

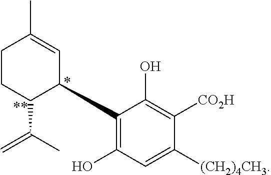

In certain embodiments, in a compound of Formula (9a)

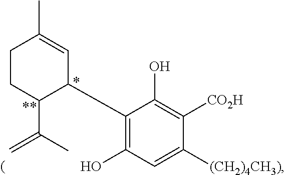

the chiral atom labeled with * at carbon 3 is of the S-configuration and a chiral atom labeled with ** at carbon 4 is of the S-configuration. In certain embodiments, a compound of Formula (9a)

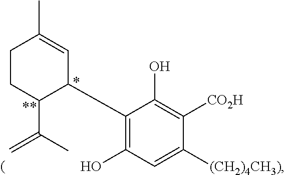

is of the formula:

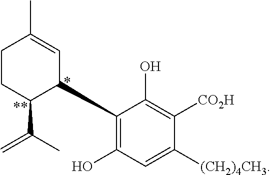

In some embodiments, as shown in FIG. 2, a TS is capable of producing a cannabinoid from the product of a PT, including, without limitation, an enzyme capable of producing a compound of Formula (9), (10), or (11):

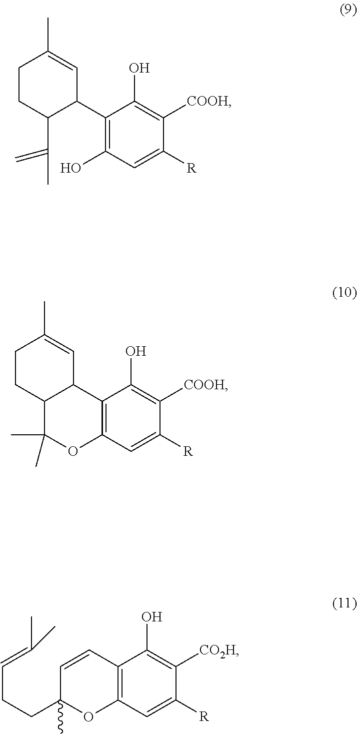

or a pharmaceutically acceptable salt, solvate, hydrate, polymorph, co-crystal, tautomer, stereoisomer, isotopically labeled derivative, or prodrug thereof, wherein R is hydrogen, optionally substituted acyl, optionally substituted alkyl, optionally substituted alkenyl, optionally substituted alkynyl, optionally substituted carbocyclyl, or optionally substituted aryl; produced from a compound of Formula (8′):

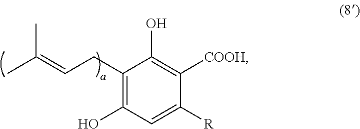

wherein a is 1, 2, 3, 4, 5, 6, 7, 8, 9, or 10; and R is hydrogen, optionally substituted acyl, optionally substituted alkyl, optionally substituted alkenyl, optionally substituted alkynyl, optionally substituted carbocyclyl, or optionally substituted aryl; or using any other substrate. In certain embodiments, a compound of Formula (8′) is a compound of Formula (8):

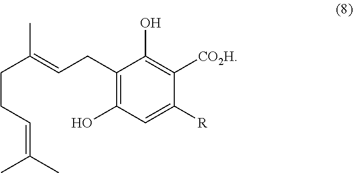

In certain embodiments, a compound of Formula (9), (10), or (11) is produced using a TS from a substrate compound of Formula (8′) (e.g., compound of Formula (8)), for example. Non-limiting examples of substrate compounds of Formula (8′) include but are not limited to cannabigerolic acid (CBGA), cannabigerovarinic acid (CBGVA), or cannabinerolic acid. In certain embodiments, at least one of the hydroxyl groups of the product compounds of Formula (9), (10), or (11) is further methylated. In certain embodiments, a compound of Formula (9) is methylated to form a compound of Formula (12):

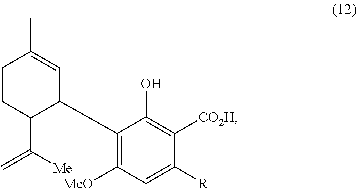

or a pharmaceutically acceptable salt, solvate, hydrate, polymorph, co-crystal, tautomer, stereoisomer, isotopically labeled derivative, or prodrug thereof.

Any of the enzymes, host cells, and methods described in this application may be used for the production of cannabinoids and cannabinoid precursors, such as those provided in Table 1. In general, the term “production” is used to refer to the generation of one or more products (e.g., products of interest and/or by-products/off-products), for example, from a particular substrate or reactant. The amount of production may be evaluated at any one or more steps of a pathway, such as a final product or an intermediate product, using metrics familiar to one of ordinary skill in the art. For example, the amount of production may be assessed for a single enzymatic reaction (e.g., conversion of a compound of Formula (8) to a compound of Formula (10) by a TS). Alternatively or in addition, the amount of production may be assessed for a series of enzymatic reactions (e.g., the biosynthetic pathway shown in FIG. 1 and/or FIG. 2). Production may be assessed by any metrics known in the art, for example, by assessing volumetric productivity, enzyme kinetics/reaction rate, specific productivity biomass-specific productivity, titer, yield, and total titer of one or more products (e.g., products of interest and/or by-products/off-products).

In some embodiments, the metric used to measure production may depend on whether a continuous process is being monitored (e.g., several cannabinoid biosynthesis steps are used in combination) or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity, biomass-specific productivity, titer, yield, and/or total titer of one or more products (e.g., products of interest and/or by-products/off-products).

Production of one or more products (e.g., products of interest and/or by-products/off-products) may be assessed indirectly, for example by determining the amount of a substrate remaining following termination of the reaction/fermentation. For example, for a TS that catalyzes the formation of products (e.g., a compound of Formula (10), including tetrahydrocannabinolic acid (THCA) (Formula (10a)) from a compound of Formula (8), including CBGA (Formula 8(a))), production of the products may be assessed by quantifying the compound of Formula (10) directly or by quantifying the amount of substrate remaining following the reaction (e.g., amount of the compound of Formula (8)). For a TS that catalyzes the formation of products (e.g., a compound of Formula (9), including cannabidiolic acid (CBDA) (Formula (9a)) from a compound of Formula (8), including CBGA (Formula 8(a))), production of the products may be assessed by quantifying the compound of Formula (9) directly or by quantifying the amount of substrate remaining following the reaction (e.g., amount of the compound of Formula (8)). For a TS that catalyzes the formation of products (e.g., a compound of Formula (11), including cannabichromenic acid (CBCA)(Formula (11a)) from a compound of Formula (8), including CBGA (Formula 8(a))), production of the products may be assessed by quantifying the compound of Formula (11) directly or by quantifying the amount of substrate remaining following the reaction (e.g., amount of the compound of Formula (8)).

In some embodiments, a TS that exhibits high production of by-products but low production of a desired product may still be used, for example if one or more amino acid substitutions, insertions, and/or deletions are introduced into the TS to shift production to the desired product, or if the TS can be expressed at locations where reaction conditions favor the production of the desired product. In some embodiments, the TS is a THCAS or has THCAS activity. Non-limiting by-products of a THCAS include compounds of Formulae (9) and (11) and a product resulting from the terpene of a compound of Formula (8) cyclizing with the other open —OH group (at carbon 1). In some embodiments, the TS is a CBDAS or has CBDAS activity. Non-limiting by-products of a CBDAS include compounds of Formulae (10) and (11) and a product resulting from the terpene of a compound of Formula (8) cyclizing with the other open —OH group (at carbon 1). In some embodiments, the TS is a CBCAS or has CBCAS activity. Non-limiting by-products of a CBCAS include compounds of Formula (9) or (10) and a product resulting from the terpene of a compound of Formula (8) cyclizing with the other open —OH group (at carbon 1). The carbons in a compound of Formula (8) may be numbered as follows:

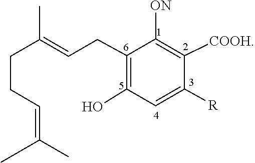

### See, e.g., Hanus et al., Nat Prod Rep. 2016 Nov. 23; 33(12):1357-1392.

In some embodiments, the production of a product (e.g., product of interest and/or by-product/off-product) by a particular TS may be assessed as relative production, for example relative to a control TS. In some embodiments, the production of a product by a particular host cell may be assessed relative to a control host cell.

In some embodiments, a TS or a host cell associated with the disclosure may be capable of producing a product at a higher titer or yield relative to a control. In some embodiments, a TS may be capable of producing a product at a faster rate (e.g., higher productivity) relative to a control. In some embodiments, a TS may have preferential binding and/or activity towards one substrate relative to another substrate. In some embodiments, a TS may preferentially produce one product relative to another product.

In some embodiments, a TS may produce at least 0.0001 μg/L, at least 0.001 μg/L, at least 0.01 μg/L, at least 0.02 μg/L, at least 0.03 μg/L, at least 0.04 μg/L, at least 0.05 μg/L, at least 0.06 μg/L, at least 0.07 μg/L, at least 0.08 μg/L, at least 0.09 μg/L, at least 0.1 μg/L, at least 0.11 μg/L, at least 0.12 μg/L, at least 0.13 μg/L, at least 0.14 μg/L, at least 0.15 μg/L, at least 0.16 μg/L, at least 0.17 μg/L, at least 0.18 μg/L, at least 0.19 μg/L, at least 0.2 μg/L, at least 0.21 μg/L, at least 0.22 μg/L, at least 0.23 μg/L, at least 0.24 μg/L, at least 0.25 μg/L, at least 0.26 μg/L, at least 0.27 μg/L, at least 0.28 μg/L, at least 0.29 μg/L, at least 0.3 μg/L, at least 0.31 μg/L, at least 0.32 μg/L, at least 0.33 μg/L, at least 0.34 μg/L, at least 0.35 μg/L, at least 0.36 μg/L, at least 0.37 μg/L, at least 0.38 μg/L, at least 0.39 μg/L, at least 0.4 μg/L, at least 0.41 μg/L, at least 0.42 μg/L, at least 0.43 μg/L, at least 0.44 μg/L, at least 0.45 μg/L, at least 0.46 μg/L, at least 0.47 μg/L, at least 0.48 μg/L, at least 0.49 μg/L, at least 0.5 μg/L, at least 0.51 μg/L, at least 0.52 μg/L, at least 0.53 μg/L, at least 0.54 μg/L, at least 0.55 μg/L, at least 0.56 μg/L, at least 0.57 μg/L, at least 0.58 μg/L, at least 0.59 μg/L, at least 0.6 μg/L, at least 0.61 μg/L, at least 0.62 μg/L, at least 0.63 μg/L, at least 0.64 μg/L, at least 0.65 μg/L, at least 0.66 μg/L, at least 0.67 μg/L, at least 0.68 μg/L, at least 0.69 μg/L, at least 0.7 μg/L, at least 0.71 μg/L, at least 0.72 μg/L, at least 0.73 μg/L, at least 0.74 μg/L, at least 0.75 μg/L, at least 0.76 μg/L, at least 0.77 μg/L, at least 0.78 μg/L, at least 0.79 μg/L, at least 0.8 μg/L, at least 0.81 μg/L, at least 0.82 μg/L, at least 0.83 μg/L, at least 0.84 μg/L, at least 0.85 μg/L, at least 0.86 μg/L, at least 0.87 μg/L, at least 0.88 μg/L, at least 0.89 μg/L, at least 0.9 μg/L, at least 0.91 μg/L, at least 0.92 μg/L, at least 0.93 μg/L, at least 0.94 μg/L, at least 0.95 μg/L, at least 0.96 μg/L, at least 0.97 μg/L, at least 0.98 μg/L, at least 0.99 μg/L, at least 1 μg/L, at least 1.1p g/L, at least 1.2 μg/L, at least 1.3 μg/L, at least 1.4 μg/L, at least 1.5 μg/L, at least 1.6 μg/L, at least 1.7 μg/L, at least 1.8 μg/L, at least 1.9 μg/L, at least 2 μg/L, at least 2.1 μg/L, at least 2.2 μg/L, at least 2.3 μg/L, at least 2.4 μg/L, at least 2.5 μg/L, at least 2.6 μg/L, at least 2.7 μg/L, at least 2.8 μg/L, at least 2.9 μg/L, at least 3 μg/L, at least 3.1 μg/L, at least 3.2 μg/L, at least 3.3 μg/L, at least 3.4 μg/L, at least 3.5 μg/L, at least 3.6 μg/L, at least 3.7 μg/L, at least 3.8 μg/L, at least 3.9 μg/L, at least 4 μg/L, at least 4.1 μg/L, at least 4.2 μg/L, at least 4.3 μg/L, at least 4.4 μg/L, at least 4.5 μg/L, at least 4.6 μg/L, at least 4.7 μg/L, at least 4.8 μg/L, at least 4.9 μg/L, at least 5 μg/L, at least 5.1 μg/L, at least 5.2 μg/L, at least 5.3 μg/L, at least 5.4 μg/L, at least 5.5 μg/L, at least 5.6 μg/L, at least 5.7 μg/L, at least 5.8 μg/L, at least 5.9 μg/L, at least 6 μg/L, at least 6.1 μg/L, at least 6.2 μg/L, at least 6.3 μg/L, at least 6.4 μg/L, at least 6.5 μg/L, at least 6.6 μg/L, at least 6.7 μg/L, at least 6.8 μg/L, at least 6.9 μg/L, at least 7 μg/L, at least 7.1 μg/L, at least 7.2 μg/L, at least 7.3 μg/L, at least 7.4 μg/L, at least 7.5 μg/L, at least 7.6 μg/L, at least 7.7 μg/L, at least 7.8 μg/L, at least 7.9 μg/L, at least 8 μg/L, at least 8.1 μg/L, at least 8.2 μg/L, at least 8.3 μg/L, at least 8.4 μg/L, at least 8.5 μg/L, at least 8.6 μg/L, at least 8.7 μg/L, at least 8.8 μg/L, at least 8.9 μg/L, at least 9 μg/L, at least 9.1 μg/L, at least 9.2 μg/L, at least 9.3 μg/L, at least 9.4 μg/L, at least 9.5 μg/L, at least 9.6 μg/L, at least 9.7 μg/L, at least 9.8 μg/L, at least 9.9 μg/L, at least 10 μg/L, at least 10.1 μg/L, at least 10.2 μg/L, at least 10.3 μg/L, at least 10.4 μg/L, at least 10.5 μg/L, at least 10.6 μg/L, at least 10.7 μg/L, at least 10.8 μg/L, at least 10.9 μg/L, at least 11 μg/L, at least 11.1 μg/L, at least 11.2 μg/L, at least 11.3 μg/L, at least 11.4 μg/L, at least 11.5 μg/L, at least 11.6 μg/L, at least 11.7 μg/L, at least 11.8 μg/L, at least 11.9 μg/L, at least 12 μg/L, at least 12.1 μg/L, at least 12.2 μg/L, at least 12.3 μg/L, at least 12.4 μg/L, at least 12.5 μg/L, at least 12.6 μg/L, at least 12.7 μg/L, at least 12.8 μg/L, at least 12.9 μg/L, at least 13 μg/L, at least 13.1 μg/L, at least 13.2 μg/L, at least 13.3 μg/L, at least 13.4 μg/L, at least 13.5 μg/L, at least 13.6 μg/L, at least 13.7 μg/L, at least 13.8 μg/L, at least 13.9 μg/L, at least 14 μg/L, at least 14.1 μg/L, at least 14.2 μg/L, at least 14.3 μg/L, at least 14.4 μg/L, at least 14.5 μg/L, at least 14.6 μg/L, at least 14.7 μg/L, at least 14.8 μg/L, at least 14.9 μg/L, at least 15 μg/L, at least 15.1 μg/L, at least 15.2 μg/L, at least 15.3 μg/L, at least 15.4 μg/L, at least 15.5 μg/L, at least 15.6 μg/L, at least 15.7 μg/L, at least 15.8 μg/L, at least 15.9 μg/L, at least 16 μg/L, at least 16.1 μg/L, at least 16.2 μg/L, at least 16.3 μg/L, at least 16.4 μg/L, at least 16.5 μg/L, at least 16.6 μg/L, at least 16.7 μg/L, at least 16.8 μg/L, at least 16.9 μg/L, at least 17 μg/L, at least 17.1 μg/L, at least 17.2 μg/L, at least 17.3 μg/L, at least 17.4 μg/L, at least 17.5 μg/L, at least 17.6 μg/L, at least 17.7 μg/L, at least 17.8 μg/L, at least 17.9 μg/L, at least 18 μg/L, at least 18.1 μg/L, at least 18.2 μg/L, at least 18.3 μg/L, at least 18.4 μg/L, at least 18.5 μg/L, at least 18.6 μg/L, at least 18.7 μg/L, at least 18.8 μg/L, at least 18.9 μg/L, at least 19 μg/L, at least 19.1 μg/L, at least 19.2 μg/L, at least 19.3 μg/L, at least 19.4 μg/L, at least 19.5 μg/L, at least 19.6 μg/L, at least 19.7 μg/L, at least 19.8 μg/L, at least 19.9 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, at least 115 μg/L, at least 120 μg/L, at least 125 μg/L, at least 130 μg/L, at least 135 μg/L, at least 140 μg/L, at least 145 μg/L, at least 150 μg/L, at least 155 μg/L, at least 160 μg/L, at least 165 μg/L, at least 170 μg/L, at least 175 μg/L, at least 180 μg/L, at least 185 μg/L, at least 190 μg/L, at least 195 μg/L, at least 200 μg/L, at least 205 μg/L, at least 210 μg/L, at least 215 μg/L, at least 220 μg/L, at least 225 μg/L, at least 230 μg/L, at least 235 μg/L, at least 240 μg/L, at least 245 μg/L, at least 250 μg/L, at least 255 μg/L, at least 260 μg/L, at least 265 μg/L, at least 270 μg/L, at least 275 μg/L, at least 280 μg/L, at least 285 μg/L, at least 290 μg/L, at least 295 μg/L, at least 300 μg/L, at least 305 μg/L, at least 310 μg/L, at least 315 μg/L, at least 320 μg/L, at least 325 μg/L, at least 330 μg/L, at least 335 μg/L, at least 340 μg/L, at least 345 μg/L, at least 350 μg/L, at least 355 μg/L, at least 360 μg/L, at least 365 μg/L, at least 370 μg/L, at least 375 μg/L, at least 380 μg/L, at least 385 μg/L, at least 390 μg/L, at least 395 μg/L, at least 400 μg/L, at least 405 μg/L, at least 410 μg/L, at least 415 μg/L, at least 420 μg/L, at least 425 μg/L, at least 430 μg/L, at least 435 μg/L, at least 440 μg/L, at least 445 μg/L, at least 450 μg/L, at least 455 μg/L, at least 460 μg/L, at least 465 μg/L, at least 470 μg/L, at least 475 μg/L, at least 480 μg/L, at least 485 μg/L, at least 490 μg/L, at least 495 μg/L, at least 500 μg/L, at least 600 μg/L, at least 700 μg/L, at least 800 μg/L, at least 900 μg/L, at least 1,000 μg/L, at least 2,000 μg/L, at least 3,000 μg/L, at least 4,000 μg/L, at least 5,000 μg/L, at least 6,000 μg/L, at least 7,000 μg/L, at least 8,000 μg/L, at least 9,000 μg/L, at least 10,000 μg/L, at least 11,000 μg/L, at least 12,000 μg/L, at least 13,000 μg/L, at least 14,000 μg/L, at least 15,000 μg/L, at least 16,000 μg/L, at least 17,000 μg/L, at least 18,000 μg/L, at least 19,000 μg/L, at least 20,000 μg/L, at least 21,000 μg/L, at least 22,000 μg/L, at least 23,000 μg/L, at least 24,000 μg/L, at least 25,000 μg/L, at least 26,000 μg/L, at least 27,000 μg/L, at least 28,000 μg/L, at least 29,000 μg/L, at least 30,000 μg/L, at least 31,000 μg/L, at least 32,000 μg/L, at least 33,000 μg/L, at least 34,000 μg/L, at least 35,000 μg/L, at least 36,000 μg/L, at least 37,000 μg/L, at least 38,000 μg/L, at least 39,000 μg/L, at least 40,000 μg/L, at least 41,000 μg/L, at least 42,000 μg/L, at least 43,000 μg/L, at least 44,000 μg/L, at least 45,000 μg/L, at least 46,000 μg/L, at least 47,000 μg/L, at least 48,000 μg/L, at least 49,000 μg/L, at least 50,000 μg/L, at least 51,000 μg/L, at least 52,000 μg/L, at least 53,000 μg/L, at least 54,000 μg/L, at least 55,000 μg/L, at least 56,000 μg/L, at least 57,000 μg/L, at least 58,000 μg/L, at least 59,000 μg/L, at least 60,000 μg/L, at least 61,000 μg/L, at least 62,000 μg/L, at least 63,000 μg/L, at least 64,000 μg/L, at least 65,000 μg/L, at least 66,000 μg/L, at least 67,000 μg/L, at least 68,000 μg/L, at least 69,000 μg/L, at least 70,000 μg/L, at least 71,000 μg/L, at least 72,000 μg/L, at least 73,000 μg/L, at least 74,000 μg/L, at least 75,000 μg/L, at least 76,000 μg/L, at least 77,000 μg/L, at least 78,000 μg/L, at least 79,000 μg/L, at least 80,000 μg/L, at least 81,000 μg/L, at least 82,000 μg/L, at least 83,000 μg/L, at least 84,000 μg/L, at least 85,000 μg/L, at least 86,000 μg/L, at least 87,000 μg/L, at least 88,000 μg/L, at least 89,000 μg/L, at least 90,000 μg/L, at least 91,000 μg/L, at least 92,000 μg/L, at least 93,000 μg/L, at least 94,000 μg/L, at least 95,000 μg/L, at least 96,000 μg/L, at least 97,000 μg/L, at least 98,000 μg/L, at least 99,000 μg/L, at least 100,000 μg/L, at least 105,000 μg/L, at least 110,000 μg/L, at least 115,000 μg/L, at least 120,000 μg/L, at least 125,000 μg/L, at least 130,000 μg/L, at least 135,000 μg/L, at least 140,000 μg/L, at least 145,000 μg/L, at least 150,000 μg/L, at least 155,000 μg/L, at least 160,000 μg/L, at least 165,000 μg/L, at least 170,000 μg/L, at least 175,000 μg/L, at least 180,000 μg/L, at least 185,000 μg/L, at least 190,000 μg/L, at least 195,000 μg/L, at least 200,000 μg/L, at least 205,000 μg/L, at least 210,000 μg/L, at least 215,000 μg/L, at least 220,000 μg/L, at least 225,000 μg/L, at least 230,000 μg/L, at least 235,000 μg/L, at least 240,000 μg/L, at least 245,000 μg/L, at least 250,000 μg/L, at least 255,000 μg/L, at least 260,000 μg/L, at least 265,000 μg/L, at least 270,000 μg/L, at least 275,000 μg/L, at least 280,000 μg/L, at least 285,000 μg/L, at least 290,000 μg/L, at least 295,000 μg/L, at least 300,000 μg/L, at least 305,000 μg/L, at least 310,000 μg/L, at least 315,000 μg/L, at least 320,000 μg/L, at least 325,000 μg/L, at least 330,000 μg/L, at least 335,000 μg/L, at least 340,000 μg/L, at least 345,000 μg/L, at least 350,000 μg/L, at least 355,000 μg/L, at least 360,000 μg/L, at least 365,000 μg/L, at least 370,000 μg/L, at least 375,000 μg/L, at least 380,000 μg/L, at least 385,000 μg/L, at least 390,000 μg/L, at least 395,000 μg/L, at least 400,000 μg/L, at least 405,000 μg/L, at least 410,000 μg/L, at least 415,000 μg/L, at least 420,000 μg/L, at least 425,000 μg/L, at least 430,000 μg/L, at least 435,000 μg/L, at least 440,000 μg/L, at least 445,000 μg/L, at least 450,000 μg/L, at least 455,000 μg/L, at least 460,000 μg/L, at least 465,000 μg/L, at least 470,000 μg/L, at least 475,000 μg/L, at least 480,000 μg/L, at least 485,000 μg/L, at least 490,000 μg/L, at least 495,000 μg/L, at least 500,000 μg/L, at least 600,000 μg/L, at least 700,000 μg/L, at least 800,000 μg/L, at least 900,000 μg/L, or at least 1,000,000 μg/L, including all values in between, of a product described herein. In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments, a TS or a host cell associated with the disclosure may be capable of producing more of an amount of one or more products than produced by a control (e.g., a positive control). In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300/o, at least 400%, at least 500%, at least 600%, at least 700%, at least 800%, at least 900%, or at least 1,000%) of the amount of one or more products produced by a control (e.g., such as a positive control). In some embodiments, a product is THCA, THCVA, CBDA, CBDVA, CBCA and/or CBCVA. In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of one or more products produced by a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments, a TS or a host cell associated with the disclosure may be capable of producing at least 0.05% (e.g., at least 0.075%, at least 0.10%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) of the titer or yield one or more products produced by a control (e.g., such as a positive control). In some embodiments, a product is THCA, THCVA, CBDA, CBDVA, CBCA and/or CBCVA. In some embodiments, a TS or a host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1⁰⁰⁰, at least 125%, at least 150%, at least 175%, at least 200%, at least 300%, at least 400%, at least 5000, at least 600%, at least 700%, at least 800%, at least 900%, or at least 1,000%) higher titer or yield of one or more products as compared to a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments, a TS or host cell associated with the disclosure may be capable of producing one or more products at a rate that is at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) the rate of a control (e.g., such as a positive control). In some embodiments, a product is THCA, THCVA, CBDA, CBDVA, CBCA and/or CBCVA. In some embodiments, a TS or host cell associated with the disclosure may be capable of producing one or more products at a rate that is at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%6, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) faster relative to a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments, a TS or host cell associated with the disclosure may be capable of producing less of an amount of one or more products than produced by a control (e.g., a positive control). In some embodiments, a TS or host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of one or more products relative to a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)). In some embodiments, a product is THCA, THCVA, CBDA, CBDVA, CBCA and/or CBCVA.

In some embodiments, a TS or host cell associated with the disclosure may be capable of producing at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) lower titer or yield of one or more products relative to a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments, a TS or host cell associated with the disclosure may be capable of producing one or more products at a rate that is at least 0.05% (e.g., at least 0.075%, at least 0.1%, at least 0.5%, at least 0.75%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) slower relative to a control (e.g., such as a positive control). In some embodiments, a product is a compound of Formula (9) (e.g., the compound of Formula (9a)). In some embodiments, a product is a compound of Formula (10) (e.g., the compound of Formula (10a)). In some embodiments, a product is a compound of Formula (11) (e.g., the compound of Formula (11a)).

In some embodiments of methods described herein involving comparison of an experimental TS to a control, the control is a wild-type reference TS. In some embodiments, the control is a wild-type C. sativa THCAS (e.g., comprising SEQ ID NO: 21 or SEQ ID NO: 284 and optionally one or more signal sequences set forth in Table 2), or a wild-type C. sativa CBDAS (e.g. comprising SEQ ID NO: 136 and optionally one or more signal sequences set forth in Table 2). In some embodiments, the control TS is identical to an experimental TS except for the presence of one or more amino acid substitutions, insertions, or deletions within the experimental TS.

In some embodiments of methods described herein involving comparison of an experimental host cell to a control host cell, the control host cell is a host cell that does not comprise a heterologous polynucleotide encoding a TS. In some embodiments, a control host cell is a wild type cell. In some embodiments, a control host cell is a host cell that comprises a heterologous polynucleotide encoding a wild-type C. sativa THCAS. In some embodiments, the control is a wild-type C. Saliva THCAS that also exhibits CBCAS activity in addition to THCAS activity. In Cannabis, the wild-type CsTHCAS is secreted into glandular trichomes. However, as described in further detail below, it may be desirable to control the localization of a cannabinoid produced by the recombinant host cell, for example to a particular cellular compartment and/or the cellular secretory pathway. Accordingly, in some embodiments, the control is a wild-type C. sativa THCAS, that also exhibits CBCAS activity, in which the native signal sequence has been removed (e.g., as set forth in SEQ ID NO: 21) and, optionally, replaced with one or more heterologous signal sequences. In some embodiments, a control host cell is a host cell that comprises a heterologous polynucleotide comprising SEQ ID NO: 22. In some embodiments, a control host cell is a host cell that comprises a heterologous polynucleotide encoding SEQ ID NO: 284 and optionally one or more signal sequences set forth in Table 2. In some embodiments, a control host cell is a host cell that comprises a heterologous polynucleotide encoding SEQ ID NO: 136 and optionally one or more signal sequences set forth in Table 2. In some embodiments, a control host cell is genetically identical to an experimental host cell except for the presence of one or more amino acid substitutions, insertions, or deletions within a TS that is heterologously exressed in the experimental host cell.

In some embodiments, a TS is capable of producing a mixture of products. For example, the mixture may comprise one or more compounds of Formula (10). In some embodiments, the mixture comprises a compound of Formula (9), Formula (10), and/or Formula (11). In some embodiments, at least approximately 50-100%, at least approximately 50-60%, at least approximately 60-70%, at least approximately 70-80/a, at least approximately 80-90%, at least approximately 90-100%, of compounds within the product mixture are compounds of Formula (10a). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (10a) than another compound of Formula (10). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (10a) than another compound of Formula (10).

In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (9a). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (9a) than another compound of Formula (9). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (9a) than another compound of Formula (9).

In some embodiments, at least approximately 50-100/o, at least approximately 50-60%, at least approximately 60-70%, at least approximately 70-80%, at least approximately 80-90%, at least approximately 90-100%, of compounds within the product mixture are compounds of Formula (11a). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (11a) than another compound of Formula (11). In some embodiments, a TS is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times less of a compound of Formula (1l a) than another compound of Formula (11).

c. Signal Peptides

Any of the enzymes described in this application, including TSs, may comprise a signal peptide. Signal peptides, also referred to as “signal sequences,” generally comprise approximately 15-30 amino acids and are involved in regulating trafficking of a newly translated protein to a particular cellular compartment and/or the cellular secretory pathway.

In some instances, a signal peptide promotes localization of an enzyme of interest. A non-limiting example of a signal peptide that promotes localization of an enzyme of interest in intracellular spaces is the MFalpha2 signal peptide. See, e.g., the signal sequence from UniProtKB—U3N2M0 (residues 1-19) and Singh et al., Nucleic Acids Res. (1983) June 25; 11(12): 4049-4063. In other instances, a signal peptide is capable of preventing a protein from being secreted from the endoplasmic reticulum (ER) and/or is capable of facilitating the return of such a protein if it is inadvertently exported. Such a signal peptide may be referred to as an “ER retentional signal.” A non-limiting example of a signal peptide that is capable of preventing a protein from being secreted from the ER and/or is capable of facilitating the return of such a protein if it is inadvertently exported is an HDEL signal peptide. See, e.g., Pelham et al., EMBO J(1988)7:1757-1762.

Non-limiting examples of signal peptides include those listed in Table 2 below. As one of ordinary skill in the art would appreciate, other signal peptides known in the art would also be compatible with aspects of the disclosure. A signal peptide may be located N-terminal or C-terminal relative to a sequence encoding an enzyme of interest. A sequence encoding an enzyme of interest may be linked to two or more signal peptides. In some embodiments, an enzyme of interest may be linked to one or more signal peptides at the N-terminus and one or more signal peptides at the C-terminus. For example, in some embodiments, the MFalpha2 signal peptide may be located N-terminal to a sequence encoding an enzyme of interest and/or the HDEL signal peptide may be located C-terminal to a sequence encoding an enzyme of interest. In other embodiments, the HDEL signal peptide may be located N-terminal to a sequence encoding an enzyme of interest and/or the MFalpha2 signal peptide may be located C-terminal to a sequence encoding an enzyme of interest.

Without wishing to be bound by any theory, it is believed that an enzyme, such as a TS enzyme, linked to the MFalpha2 signal peptide and/or the HDEL signal peptide will be localized to intracellular locations associated with the secretory pathway, such as the ER and/or the Golgi apparatus. One or more of the conditions of the secretory pathway are believed to contribute to improved activity of TS enzymes derived from C. sativa. For example, the ER and Golgi apparatus are oxidative environments, which may assist in the formation of disulphide bridges. Without wishing to be bound by any theory, signal peptides and the resulting intracellular localization of proteins containing the signal peptides may differentially impact the stability and/or half-life of proteins.

In some embodiments, a signal peptide comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 3-4, 16-19, 35, 44, 135, 314-319, and 607-637.

In some embodiments, a signal peptide comprises a sequence that differs by no more than 1, 2, 3, 4, 5, 6, 7, 8, 9, 10, 11, 12, 13, 14, 15, 16, 17, or 18 amino acids from any of SEQ ID NOs: 3, 4, or 16. In some embodiments, a signal peptide comprises no more than 1, 2, 3, 4, 5, 6, 7, 8, 9, 10, 11, 12, 13, 14, 15, 16, 17, or 18 amino acid substitutions, insertions, additions, or deletions relative to the sequence of SEQ ID NOs: 3, 4, or 16. In some embodiments, a signal peptide comprises SEQ ID NO: 16 or a sequence that has no more than 2 amino acid substitutions, insertions, additions, or deletions relative to the sequence of SEQ ID NO: 16. In some embodiments, a signal peptide comprises a protein sequence that differs by no more than 1, 2 or 3 amino acids from SEQ ID NO: 17. In some embodiments, a signal peptide comprises SEQ ID NO: 17 or a sequence that has no more than one amino acid substitution, insertion, addition, or deletion relative to the sequence of SEQ ID NO: 17.

A signal peptide that is located at the N-terminus of a sequence encoding an enzyme of interest may comprise a methionine at the N-terminus of the signal peptide. In some embodiments, a methionine is added to a signal peptide if the signal peptide will be located at the N-terminus of a sequence encoding an enzyme of interest. In some embodiments, a signal peptide that is normally associated with an enzyme of interest (e.g., a naturally occurring signal peptide that is present in a naturally occurring enzyme of interest) may be removed or replaced with one or more different signal peptides that are suitable for targeting the enzyme to a particular cellular compartment in a host cell of interest.

TABLE 2
Non-limiting examples of signal peptides

| | Amino | Non-limiting examples |
|---|---|---|
| | acid | of corresponding |
| Name | sequence | nucleic acid sequences |
| C. | NCSAFSFWF | aattgctcagcattttccttttggtt |
| sativa | VCKIIFFFL | tgtttgcaaaataatatttttctttc |
| THCAS | SFNIQISIA | tctcattcaatatccaaatttcaata |
| native | (SEQ ID | (SEQ ID NO: 3) |
| signal | NO: 4) | |
| peptide | | |
| C. | NCSAFSFWF | aactgcagcgcgtttagcttttggtt |
| sativa | VCKIIFFFL | tgtgtgcaaaattatttttttttttc |
| THCAS | SFHIQISIA | tgagctttcatattcagattagcatt |
| native | (SEQ ID | gcg |
| signal | NO: 317) | (SEQ ID NO: 135) |
| peptide | | aattgctcagcattttccttctggtt |
| | | cgtctgtaagattatctttttctttc |
| | | tttctttccacatacaaatctcgatt |
| | | gccaa |
| | | (SEQ ID NO: 316) |
| C. | NCSTFSFWF | aattgctcaacattctccttttggtt |
| sativa | VCKIIFFFL | tgtttgcaaaataatatttttctttc |
| THCAS | SFNIQISIA | tctcattcaatatccaa atttcaat |
| native | (SEQ ID | agct |
| signal | NO: 319) | (SEQ ID NO: 318) |
| peptide | | |
| C. | KCSTFSFWF | aaatgcagcacctttagcttttggtt |
| sativa | VCKIIFFFF | tgtgtgcaaaattatttttttttttt |
| CBDAS | SFNIQTSIA | ttagctttaacattcagaccagcatt |
| native | (SEQ ID | gcg |
| signal | NO: 315) | (SEQ ID NO: 44) |
| peptide | | aagtgctcaacattctccttttggtt |
| | | tgtttgcaagataatatttttctttt |
| | | tctcattcaatatccaaacttccatt |
| | | gct |
| | | (SEQ ID NO: 314) |
| MFalpha2 | KFISTFLTF | aagtttatcagtaccttcttgacctt |
| | ILAAVSVTA | tatcttggccgctgtctccgtaaccg |
| | (SEQ ID | ct |
| | NO: 16) | (SEQ ID NO: 18) |
| | | aaattcatttctacctttctcacttt |
| | | tattttagcggccgtttctgtcactg |
| | | ct |
| | | (SEQ ID NO: 35) |
| HDEL | HDEL | catgatgaatta |
| | (SEQ ID | (SEQ ID NO: 19) |
| | NO: 17) | cacgatgaattg |
| | | (SEQ ID NO: 607) |
| YLR120C | KLKTVRSAV | aaactgaaaactgtaagatctgcggt |
| native | LSSLFASQV | cctttcgtcactctttgcatcgcagg |
| signal | LG | ttctcggt |
| peptide | (SEQ ID | (SEQ ID NO: 609) |
| | NO: 608) | |
| Osm1p | IRSVRRVFI | attagatctgtgagaagggttttcat |
| native | YVSIFVLII | ttacgtctcaatattcgtattgataa |
| leader | VLKRTLSGT | tagttttgaaaagaacattaagtggc |
| peptide | DQTS | acagatcaaacgtca |
| | (SEQ ID | (SEQ ID NO: 611) |
| | NO: 610) | |
| Sf | IRLTVFLTA | atcagattgaccgttttcttgaccgc |
| leader | VFAAVASC | tgtttttgctgctgttgcttcttgt |
| peptide | (SEQ ID | (SEQ ID NO: 613) |
| | NO: 612) | |
| Ost1 | RQVWFSWIV | aggcaggtttggttctcttggattgt |
| leader | GLFLCFFNV | gggattgttcctatgttttttcaacg |
| peptide | SSA | tgtcttctgct |
| | (SEQ ID | (SEQ ID NO: 615) |
| | NO: 614) | |
| YDR456W | LSKVLLNIA | ctatccaaggtattgctgaatatagc |
| native | FKVLLTTAK | tttcaaggtgctgttaaccaccgcca |
| signal | R | agaga |
| peptide | (SEQ ID | (SEQ ID NO: 617) |
| | NO: 616) | |
| YNL121C | KSFITRNKT | aagagcttcattacaaggaacaagac |
| native | AILATVAAT | agccattttggcaaccgttgctgcta |
| signal | GTAIG | caggtactgccatcggt |
| peptide | (SEQ ID | (SEQ ID NO: 619) |
| | NO: 618) | |
| OSM1- | IRSVRRVFI | attagatctgtgagaagggttttcat |
| leader | YVSIFVLII | ttacgtctcaatattcgtattgataa |
| peptide | VLKRLLLGT | tagttttgaaaagattattattgggc |
| T23L, | DQTS | acagatcaaacgtca |
| S25L | (SEQ ID | (SEQ ID NO: 621) |
| | NO: 620) | |
| ERG11 | SATKSIVGE | tctgctaccaagtcaatcgttggaga |
| leader | ALEYVNIGL | ggcattggaatacgtaaacattggtt |
| peptide | SHFLALPLA | taagtcatttcttggctttaccattg |
| | QRISLIIII | gcccaaagaatctctttgatcataat |
| | PFIYNIVWQ | aattcctttcatttacaatattgtat |
| | LLYSLRKDR | ggcaattactatattctttgagaaag |
| | PP | gaccgtccacct |
| | (SEQ ID | (SEQ ID NO: 623) |
| | NO: 622) | |
| PRC1 | KAFTSLLCG | aaagcattcaccagtttactatgtgg |
| signal | LGLSTTLAK | actaggcctgtccactacactcgcta |
| peptide | AISLQRPLG | aggccatctcattgcaaagaccgttg |
| (1-111) | LDKDVLLQA | ggtctagataaggacgttttgctgca |
| | AEKFGLDLD | agctgcggaaaaatttggtttggacc |
| | LDHLLKELD | tcgacctggatcatctcttgaaggag |
| | SNVLDAWAQ | ttggactccaatgtattggacgcttg |
| | IEHLYPNQV | ggcccaaatagagcatttgtacccaa |
| | MSLETSTKP | accaggttatgagccttgaaacttcc |
| | KFPEAIKTK | actaagccaaaattccctgaagcaat |
| | KDWDFVVKN | caaaacgaagaaagactgggactttg |
| | DAIENYQLR | tggtcaagaatgacgcaattgaaaac |
| | VN | tatcagcttcgtgtcaac |
| | (SEQ ID | (SEQ ID NO: 625) |
| | NO: 624) | |
| PEP4 | FSLKALLPL | Ttcagcttgaaagcattattgccatt |
| signal | ALLLVSANQ | ggccttgttgttggtcagcgccaacc |
| peptide | VAAKV | aagttgctgcaaaagtc |
| (1-24) | (SEQ ID | (SEQ ID NO: 627) |
| | NO: 626) | |
| PEP4 | FSLKALLPL | ttcagcttgaaagcattattgccatt |
| signal | ALLLVSANQ | ggccttgttgttggtcagcgccaacc |
| peptide | VAAKVHKAK | aagttgctgcaaaagtccacaaggct |
| (2-76) | IYKHELSDE | aaaatttataaacacgagttgtccga |
| | MKEVTFEQH | tgagatgaaagaagtcactttcgagc |
| | LAHLGQKYL | aacatttagctcatttaggccaaaag |
| | TQFEKANPE | tacttgactcaatttgagaaagctaa |
| | VVFSREHPF | ccccgaagttgttttttctagggagc |
| | FTE | atcctttcttcactgaa |
| | (SEQ ID | (SEQ ID NO: 629) |
| | NO: 628) | |
| KLD | KLD | aagctggac |
| | (SEQ ID | (SEQ ID NO: 631) |
| | NO: 630) | |
| Tail | MVYIGIAIF | atggtttatattggtatcgctatttt |
| anchor | LFLVGLFMK | tttgtttttggttggcctttttatga |
| UBC6 | (SEQ ID | aa |
| | NO: 632) | (SEQ ID NO: 633) |
| PTS1 | SKL | agcaaacta |
| signal | (SEQ ID | (SEQ ID NO: 635) |
| peptide | NO: 634) | |
| (SKL | | |
| from | | |
| C1T2) | | |
| CVIA | CVIA | tgtgttattgct |
| from | (SEQ ID | (SEQ ID NO: 637) |
| MFa1 | NO: 636) | |

In some embodiments, a TS is a tetrahydrocannabinolic acid synthase (THCAS), a cannabidiolic acid synthase (CBDAS), and/or a cannabichromenic acid synthase (CBCAS). As one of ordinary skill in the art would appreciate a TS could be obtained from any source, including naturally occurring sources and synthetic sources (e.g., a non-naturally occurring TS).

### Tetrahydrocannabinolic Acid Synthase (THCAS)

A host cell described in this application may comprise a TS that is a tetrahydrocannabinolic acid synthase (THCAS). As used in this application “tetrahydrocannabinolic acid synthase (THCAS)” or “Δ¹-tetrahydrocannabinolic acid (THCA) synthase” refers to an enzyme that is capable of catalyzing oxidative cyclization of a prenyl moiety (e.g., terpene) of a compound of Formula (8) to produce a ring-containing product (e.g., heterocyclic ring-containing product, carbocyclic-ring containing product) of Formula (10). In certain embodiments, a THCAS refers to an enzyme that is capable of producing Δ9-tetrahydrocannabinolic acid (Δ9-THCA), THCA, Δ9-Tetrahydro-cannabivannic acid A (A9-THCVA-C3 A), THCVA, THCPA, or a compound of Formula 10(a), from a compound of Formula (8). In certain embodiments, a THCAS is capable of producing Δ⁹-tetrahydrocannabinolic acid (Δ⁹-THCA, THCA, or a compound of Formula 10(a)). In certain embodiments, a THCAS is capable of producing Δ9-tetrahydrocannabivarinic acid (A9-THCVA, THCVA, or a compound of Formula 10 where R is n-propyl).

In some embodiments, a THCAS may catalyze the oxidative cyclization of substrates, such as 3-prenyl-2,4-dihydroxy-6-alkylbenzoic acids. In some embodiments, a THCAS may use cannabigerolic acid (CBGA) as a substrate. In some embodiments, the THCAS produces A9-THCA from CBGA. In some embodiments, a THCAS may catalyze the oxidative cyclization of cannabigerovarinic acid (CBGVA). In some embodiments, a THCAS exhibits specificity for CBGA substrates as compared to other substrates. In some embodiments, a THCAS may use a compound of Formula (8) of FIG. 2 where R is C4 alkyl (e.g., n-butyl) or R is C7 alkyl (e.g., n-heptyl) as a substrate. In some embodiments, a THCAS may use a compound of Formula (8) where R is C4 alkyl (e.g., n-butyl) as a substrate. In some embodiments, a THCAS may use a compound of Formula (8) of FIG. 2 where R is C7 alkyl (e.g., n-heptyl) as a substrate. In some embodiments, the THCAS exhibits specificity for substrates that can result in THCP as a product.

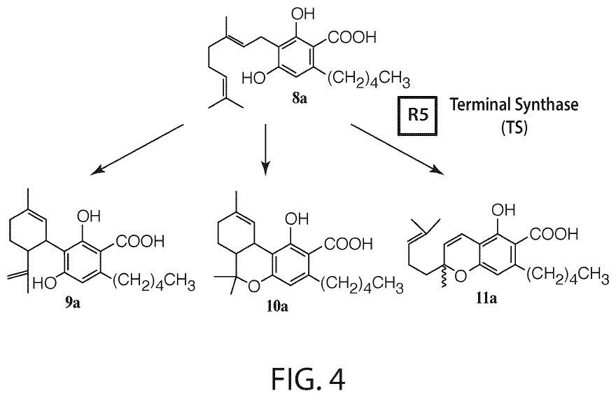

In some embodiments, a THCAS is from C. sativa. C. sativa THCAS performs the oxidative cyclization of the geranyl moiety of Cannabigerolic Acid (CBGA) (FIG. 4 Structure 8a) to form Tetrahydrocannabinolic Acid (FIG. 4 Structure 10a) using covalently bound flavin adenine dinucleotide (FAD) as a cofactor and molecular oxygen as the final electron acceptor. THCAS was first discovered and characterized by Taura et al. (JACS. 1995) following extraction of the enzyme from the leaf buds of C. sativa and confirmation of its THCA synthase activity in vitro upon the addition of CBGA as a substrate. A crystal structure of the enzyme published by Shoyama et al. (J Mol Biol. 2012 Oct. 12:423(1):96-105) revealed that the enzyme covalently binds to a molecule of the cofactor FAD. See also, e.g., Sirikantarams et al., J. Biol. Chem. 2004 Sep. 17; 279(38):39767-39774. There are several THCAS isozymes in C. sativa.

In some embodiments, a C. sativa THCAS (Uniprot KB Accession No.: I1V0C5) comprises the amino acid sequence shown below, in which the signal peptide is underlined and bolded:

(SEQ ID NO: 20)
MNCSAFSFWFVCKIIFFFLSFNIQISIANPQENFLKCF
SEYIPNNPANPKFIYTQHDQLYMSVLNSTIQNLRFTSDTTPKPLVIVTPS
NVSHIQASILCSKKVGLQIRTRSGGHDAEGMSYISQVPFVVVDLRNMHSI
KIDVHSQTAWVEAGATLGEVYYWINEKNENFSFPGGYCPTVGVGGHFSGG
GYGALMRNYGLAADNIIDAHLVNVDGKVLDRKSMGEDLFWAIRGGGGENF
GIIAAWKIKLVAVPSKSTIFSVKKNMEIHGLVKLFNKWQNIAYKYDKDLV
LMTHFITKNITDNHGKNKTTVHGYFSSIFHGGVDSLVDLMNKSFPELGIK
KTDCKEFSWIDTTIFYSGVVNFNTANFKKEILLDRSAGKKTAFSIKLDYV
KKPIPETAMVKILEKLYEEDVGVGMYVLYPYGGIMEEISESAIPFPHRAG
IMYELWYTASWEKQEDNEKHINWVRSVYNFTTPYVSQNPRLAYLNYRDLD
LGKTNPESPNNYTQARIWGEKYFGKNFNRLVKVKTKADPNNFFRNEQSIP
PLPPHHH.

In some embodiments, a THCAS comprises the sequence shown below:

(SEQ ID NO: 21)
NPQENFLKCFSEYIPNNPANPKFIYTQHDQLYMSVLNSTIQNLRFTSDTT
PKPLVIVTPSNVSHIQASILCSKKVGLQIRTRSGGHDAEGMSYISQVPFV
VVDLRNMHSIKIDVHSQTAWVEAGATLGEVYYWINEKNENFSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGVGMYVLYPYGGIMEEISE
SAIPFPHRAGIMYELWYTASWEKQEDNEKHINWVRSVYNFTTPYVSQNPR
LAYLNYRDLDLGKTNPESPNNYTQARIWGEKYFGKNFNRLVKVKTKADPN
NFFRNEQSIPPLPPHHH.

A non-limiting example of a nucleotide sequence encoding SEQ ID NO: 21 is:

(SEQ ID NO: 22)
aacccgcaagaaaactttctaaaatgcttttctgaatacattcctaacaa
ccctgccaacccgaagtttatctacacacaacacgatcaattgtatatga
gcgtgttgaatagtacaatacagaacctgaggtttacatccgacacaacg
ccgaaaccgctagtgatcgtcacaccctccaacgtaagccacattcaggc
aagcattttatgcagcaagaaagtcggactgcagataaggacgaggtccg
gaggacacgacgccgaagggatgagctatatctcccaggtaccttttgtg
gtggtagacttgagaaatatgcactctatcaagatagacgttcactccca
aaccgcttgggttgaggcgggagccacccttggtgaggtctactactgga
tcaacgaaaagaatgaaaattttagctttcctgggggatattgcccaact
gtaggtgttggcggccacttctcaggaggcggttatggggccttgatgcg
taactacggacttgcggccgacaacattatagacgcacatctagtgaatg
tagacggcaaagttttagacaggaagagcatgggtgaggatcttttttgg
gcaattagaggggagggggagaaaattttggaattatcgctgcttggaaa
attaagctagttgcggtaccgagcaaaagcactatattctctgtaaaaaa
gaacatggagatacatggtttggtgaagctttttaataagtggcaaaaca
tcgcgtacaagtacgacaaagatctggttctgatgacgcattttataacg
aaaaatatcaccgacaaccacggaaaaaacaaaaccacagtacatggcta
cttctctagtatatttcatgggggagtcgattctctggttgatttaatga
acaaatcattcccagagttgggtataaagaagacagactgtaaggagttc
tcttggattgacacaactatattctattcaggcgtagtcaactttaacac
ggcgaatttcaaaaaagagatccttctggacagatccgcaggtaagaaaa
ctgcgttctctatcaaattggactatgtgaagaagcctattcccgaaacc
gcgatggtcaagatacttgagaaattatacgaggaagatgtgggagttgg
aatgtacgtactttatccctatggtgggataatggaagaaatcagcgaga
gcgccattccatttccccatcgtgccggcatcatgtacgagctgtggtat
actgcgagttgggagaagcaagaagacaacgaaaagcacattaactgggt
cagatcagtttacaatttcaccaccccatacgtgtcccagaatccgcgtc
tggcttacttgaactaccgtgatcttgacctgggtaaaacgaacccggag
tcacccaacaattacactcaagctagaatctggggagagaaatactttgg
gaagaacttcaacaggttagtaaaggttaaaaccaaggcagatccaaaca
cacttttttagaaatgaacaatcattcccccgctacccccgcaccatca
c.

In some embodiments, a THCAS comprises the amino acid sequence shown below, in which signal peptides are underlined and bolded:

(SEQ ID NO: 23)
MKFISTFLTFILAAVSVTANPQENFLKCFSEYIPNNP
ANPKFIYTQHDQLYMSVLNSTIQNLRFTSDTTPKPLVIVTPSNVSHIQAS
ILCSKKVGLQIRTRSGGHDAEGMSYISQVPFVVVDLRNMHSIKIDVHSQT
AWVEAGATLGEVYYWINEKNENFSFPGGYCPTVGVGGHFSGGGYGALMRN
YGLAADNIIDAHLVNVDGKVLDRKSMGEDLFWAIRGGGGENFGIIAAWKI
KLVAVPSKSTIFSVKKNMEIHGLVKLFNKWQNIAYKYDKDLVLMTHFITK
NITDNHGKNKTTVHGYFSSIFHGGVDSLVDLMNKSFPELGIKKTDCKEFS
WIDTTIFYSGVVNFNTANFKKEILLDRSAGKKTAFSIKLDYVKKPIPETA
MVKILEKLYEEDVGVGMYVLYPYGGIMEEISESAIPFPHRAGIMYELWYT
ASWEKQEDNEKHINWVRSVYNFTTPYVSQNPRLAYLNYRDLDLGKTNPES
PNNYTQARIWGEKYFGKNFNRLVKVKTKADPNNFFRNEQSIPPLPPHHHH
DEL.

A non-limiting example of a nucleotide sequence encoding SEQ ID NO: 23, in which sequences encoding signal peptides are underlined and bolded, is shown below:

(SEQ ID NO: 24)
atgaagtttatcagtaccttcttgacctttatct
tggccgctgtctccgtaaccgctaacccgcaaga
aaactttctaaaatgcttttctgaatacattcctaacaaccctgccaacc
cgaagtttatctacacacaacacgatcaattgtatatgagcgtgttgaat
agtacaatacagaacctgaggtttacatccgacacaacgccgaaaccgct
agtgatgtcacaccctccaacgtaagccacattcaggcaagcattttatg
cagcaagaaagtcggactgcagataaggacgaggtccggaggacacgacg
ccgaagggatgagctatatctcccaggtaccttttgtggtggtagacttg
agaaatatgcactctatcaagatagacgttcactcccaaaccgcttgggt
tgaggcgggagccacccttggtgaggtctactactggatcaacgaaaaga
atgaaaattttagctttcctgggggatattgcccaactgtaggtgttggc
ggccacttctcaggaggcggttatggggccttgatgcgtaactacggact
tgcggccgacaacattatagacgcacatctagtgaatgtagacggcaaag
ttttagacaggaagagcatgggtgaggatcttttttgggcaattagaggc
ggagggggagaaaattttggaattatcgctgcttggaaaattaagctagt
tgcggtaccgagcaaaagcactatattctctgtaaaaaagaacatggaga
tacatggtttggtgaagctttttaataagtggcaaaacatcgcgtacaag
tacgacaaagatctggttctgatgacgcattttataacgaaaaatatcac
cgacaaccacggaaaaaacaaaaccacagtacatggctacttctctagta
tatttcatgggggagtcgattctctggttgatttaatgaacaaatcattc
ccagagttgggtataaagaagacagactgtaaggagttctcttggattga
cacaactatattctattcaggcgtagtcaactttaacacggcgaatttca
aaaaagagatccttctggacagatccgcaggtaagaaaactgcgttctct
atcaaattggactatgtgaagaagcctattcccgaaaccgcgatggtcaa
gatacttgagaaattatacgaggaagatgtgggagttggaatgtacgtac
tttatccctatggtgggataatggaagaaatcagcgagagcgccattcca
tttccccatcgtgccggcatcatgtacgagctgtggtatactgcgagttg
ggagaagcaagaagacaacgaaaagcacattaactgggtcagatcagttt
acaatttcaccaccccatacgtgtcccagaatccgcgtctggcttacttg
aactaccgtgatcttgacctgggtaaaacgaacccggagtcacccaacaa
ttacactcaagctagaatctggggagagaaatactttgggaagaacttca
acaggttagtaaaggttaaaaccaaggcagatccaaacaacttttttaga
aatgaacaatccattcccccgctacccccgcaccatcac
catgatgaatta.

In some embodiments, a C. sativa THCAS comprises the amino acid sequence set forth in UniProtKB—Q8GTB6 (SEQ ID NO: 14) in which the signal peptide is underlined and bolded:

(SEQ ID NO: 14)
MNCSAFSFWFVCKIIFFFLSFHIQISIA
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLENKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGAGMYVLYPYGGIMEEISE
SAIPFPHRAGIMYELWYTASWEKQEDNEKHINWVRSVYNFTTPYVSQNPR
LAYLNYRDLDLGKTNHASPNNYTQARIWGEKYFGKNFNRLVKVKTKVDPN
NFFRNEQSIPPLPPHHH

In some embodiments, a THCAS comprises the sequence shown below:

(SEQ ID NO: 284)
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGAGMYVLYPYGGIMEEISE
SAIPFPHRAGIMYELWYTASWEKQEDNEKHINWVRSVYNFTTPYVSQNPR
LAYLNYRDLDLGKTNHASPNNYTQARIWGEKYFGKNFNRLVKVKTKVDPN
NFFRNEQSIPPLPPHHH.

A non-limiting example of a nucleic acid sequence encoding SEQ ID NO: 284 for expression in S. cerevisiae is:

(SEQ ID NO: 254)
aacccacgtgagaactttttgaaatgtttctctaagcatatccctaacaa
tgtggctaacccaaagttagtttacacacagcacgatcaactatatatga
gcattttgaactccactatccaaaatctgaggttcattagtgacaccacg
cccaaaccattggttattgtaaccccttctaataactctcatatccaagc
tactatattatgttccaagaaggtcggcttgcaaattagaactagatcag
gaggtcacgatgctgaaggtatgtcatacatttcccaagttccattcgtt
gttgtagacctaagaaatatgcactctatcaaaattgatgtccattctca
aacagcctgggtcgaagctggtgctaccttgggtgaagtttattactgga
ttaacgaaaagaatgaaaacttgtcgttcccaggtggttactgtccaacc
gtgggtgttggtggacactttagcggtggtggatacggcgccttgatgag
aaactatggtttagctgctgacaatatcatcgatgcacaccttgtcaacg
ttgatggtaaggtattggacagaaaatcaatgggtgaagacttgttctgg
gctataagaggtggcggtggtgagaactttggtatcatcgctgcatggaa
gattaagttagttgccgtcccatctaagtccactatcttcagtgttaaaa
agaacatggaaattcatggtttggtcaagctatttaataaatggcagaac
atcgcttacaagtacgataaggacttagttttgatgacccattttataac
taaaaacattactgataaccacggtaaaaataagactacagttcacggtt
atttctcctctatcttccatggtggagttgactctctggtcgacctaatg
aacaagtccttcccagaattgggtattaagaagactgattgcaaagaatt
ttcatggatcgataccaccattttctactctggcgtggttaactttaata
cggctaactttaagaaggaaatattgttagaccgttcggccggtaagaaa
accgctttttctataaagttggattatgttaagaaacctattcctgaaac
agccatggtcaagatcttagaaaaattgtacgaagaggatgtaggagctg
gtatgtacgttctttacccatatggtggtattatggaagaaatatctgag
tctgccattccattcccacatagggcaggcattatgtacgaattgtggta
tactgctagctgggaaaagcaagaagacaatgaaaaacacataaattggg
ttagaagtgtatacaatttcactaccccctacgtcagccaaaacccaaga
ttggcctatctaaactaccgtgacctggacctaggtaaaactaaccacgc
ttcaccaaacaactacacccaagctagaatctggggagagaagtatttcg
gtaagaatttcaacagattggtcaaagtgaagaccaaggtcgatccaaat
aattttttcagaaacgagcaatctattcccccattaccaccacatcatca
c

In some embodiments, a THCAS comprises each of: SEQ ID NO: 284; the MFalpha2 signal peptide; and the HDEL signal peptide. In some embodiments, such a THCAS comprises the amino acid sequence shown below, in which signal peptides are underlined and bolded:

(SEQ ID NO: 1220)
MKFISTFLTFILAAVSVTANPRENFLKCFSKHIPNNVANP
KLVYTQHDQLYMSILNSTIQNLRFISDTTPKPLVIVTPSNNSHIQATILC
SKKVGLQIRTRSGGHDAEGMSYISQVPFVVVDLRNMHSIKIDVHSQTAWV
EAGATLGEVYYWINEKNENLSFPGGYCPTVGVGGHFSGGGYGALMRNYGL
AADNIIDAHLVNVDGKVLDRKSMGEDLFWAIRGGGGENFGIIAAWKIKLV
AVPSKSTIFSVKKNMEIHGLVKLFNKWQNIAYKYDKDLVLMTHFITKNIT
DNHGKNKTTVHGYFSSIFHGGVDSLVDLMNKSFPELGIKKTDCKEFSWID
TTIFYSGVVNFNTANFKKEILLDRSAGKKTAFSIKLDYVKKPIPETAMVK
ILEKLYEEDVGAGMYVLYPYGGIMEEISESAIPFPHRAGIMYELWYTASW
EKQEDNEKHINWVRSVYNFTTPYVSQNPRLAYLNYRDLDLGKTNHASPNN
YTQARIWGEKYFGKNFNRLVKVKTKVDPNNFFRNBQSIPPLPPHHH
HDEL.

Additional non-limiting examples of THCAS enzymes may also be found in U.S. Pat. No. 9,512,391 and US Publication No. 2018/0179564, which are incorporated by reference in this application in their entireties.

In some embodiments, a THCAS comprises the amino acid sequence set forth in SEQ ID NO: 320:

(SEQ ID NO: 320)
MNCSAFSFWFVCKIIFFFLSFHIQISIANPRENFLKCF
SKHIPNNVANPKLVYTQHDQLYMSILNSTIQNLRFISDTTPKPLVIVTPS
NNSHIQATILCSKKVGLQIRTRSGGHDAEGMSYISQVPFVVVDLRNMHSI
KIDVHSQTAWVEAGATLGEVYYWINEKNENLSFPGGYCPTVGVGGHFSGG
GYGALMRNYGLAADNIIDAHLVNVDGKVLDRKSMGEDLFWAIRGGGGENF
GIIAAWKIKLVAVPSKSTIFSVKKNMEIHGLVKLFNKWQNIAYKYDKDLV
LMTHFITKNITDNHGKNKTTVHGYFSSIFHGGVDSLVDLMNKSFRELGIK
KTDCKELSWIDTTIFYSGVVNYNTANFKKEILLDRSAGKKTAFSIKLDYV
KKPIPETAMVKILEKLYEEDVGAGMYVLYPYGGIMEEISESAIPFPHRAG
IMYELWYTASWEKQEDNEKHINWVRSVYNFTTPYVSQNPRLAYLNYRDLD
LGKTNHASPNNYTQARIWGEKYFGKNFNRLVKVKTKVDPNNFFRNEQSIP
PLPPHHH.

In some embodiments, a THCAS comprises the amino acid sequence set forth in SEQ ID NO: 321:

(SEQ ID NO: 321)
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFRELGIKKTDCKELSWIDTTIFYSGVVNYNTANFKKEILLDRSAGKK
TAFSIKLDYVKKPIPETAMVKILEKLYEEDVGAGMYVLYPYGGIMEEISE
SAIPFPHRAGIMYELWYTASWEKQEDNEKHINWVRSVYNFTTPYVSQNPR
LAYLNYRDLDLGKTNHASPNNYTQARIWGEKYFGKNFNRLVKVKTKVDPN
NFFRNEQSIPPLPPHHH

In some embodiments, a THCAS does not comprise the sequence of SEQ ID NOs: 20, 21, 22, 23, 24, 14, 284, 254, 1220, 320 or 321. In some embodiments, a control TS comprises the sequence of any one of SEQ ID NOs: 20, 21, 22, 23, 24, 14, 284, 254, 1220, 320 or 321.

As described in the Examples section, novel THCAS enzymes were identified in this disclosure that are capable of catalyzing the conversion of a compound of Formula (8) to produce a compound of Formula (10) and that can be functionally expressed in host cells. Without being bound by a particular theory, the novel THCAS enzymes disclosed in this application may be useful for engineering to alter the activity and/or abundance of the THCAS (e.g., change the product profile, substrate profile, and/or kinetics (e.g., Kcat/Vmax and/or Kd) of the TS).

In some embodiments, a THCAS comprises the amino acid sequence shown below:

(SEQ ID NO: 37)
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIRLVAVPSRATIFSVKRNMEIHGLVKLFNKWQN
IAYKYDKDLLLMTHFITRNIIDNQGKNKTTVHGYFSCIFHGGVDSLVNLM
NKSYPELGIKKTDCKEFSWIDTTIFYSGVVNFNTANFKKEILLDRSAGKK
TAFSIKLDYVKKPIPETAMVKILEKLYEEDVGVGMYVLYPYGGIMEEISE
SAIPFPHRAGIMYELWYTASWEKQEDNEKHINWVRSVYNFTTPYVSQNPR
LAYLNYRDLDLGKTNPESPNNYTQARIWGEKYFGKNFNRLVKVKTKADPN
NFFRNEQSIPPLPPHHH.

A non-limiting example of a nucleic acid sequence encoding SEQ ID NO: 37 for expression in S. cerevisiae is:

(SEQ ID NO: 28)
aatcctagagaaaactttctgaaatgcttctctaaacacattcccaataa
tgttgctaatcctaaactggtatatacccaacacgatcaattgtacatgt
ctatcttgaacagcacgatacagaacttgagattcatatcagatacgaca
ccaaagcctttggttattgttacccctagtaataatagtcatatccaggc
tacaatattgtgtagtaagaaagtgggcctacaaataagaaccagatcag
gtggtcatgatgcggaaggcatgtcttatatttctcaggtaccatttgtg
gtggttgatttgagaaacatgcacagtatcaagatagacgttcacagtca
aacggcatgggtggaggcgggtgctacattaggtgaagtttattactgga
tcaacgaaaagaatgaaaatctgtctttcccaggcggctactgtcctaca
gttggcgttggcgggcatttctctggcggtggttatggagccttgatgag
aaactatggcttggccgcagataatataatcgacgctcacttggttaacg
ttgatggtaaggtccttgatagaaaatccatgggtgaagatttgttttgg
gcaattagggggggtggtggtgaaaattttggaattattgcggcctggaa
aattaggttggtagcagttccttccagggcaaccattttttctgttaaga
gaaacatggaaattcatggcctggtgaaattgtttaacaagtggcagaac
atcgcatacaagtacgataaagatttgttgttaatgacccactttattac
caggaacattattgataaccaaggtaaaaataagaccaccgtccatggat
acttttcgtgcatatttcatggtggtgttgatagtctagttaatttaatg
aacaaatcttatcctgaactagggatcaaaaaaactgattgcaaggaatt
ctcttggatagacacaacaattttttacagtggtgtcgtcaatttcaata
cagcaaattttaagaaagaaatattattagataggagtgcaggcaaaaag
acggctttttctattaaactagattatgttaagaagccaattccagagac
agcaatggtaaagatcctagagaaactatacgaagaggatgtcggagtcg
gaatgtacgttctttatccatatgggggtattatggaggagatatccgaa
agcgcaatcccatttccccatagagccggaattatgtatgaattgtggta
caccgcttcatgggagaagcaggaggataacgaaaagcacataaattggg
taaggtcagtttacaattttacaacaccatacgtctcacagaatccgaga
ttggcttatttgaactacagagacttggacctgggaaagacaaacccaga
gagcccaaacaattacactcaagcaaggatttggggtgaaaagtacttcg
gtaaaaatttcaataggctggtgaaagtcaagactaaggcggatccgaac
aacttcttcaggaacgaacaatccattccaccacttcctccacatcacca
c.

In some embodiments, a THCAS comprises each of: SEQ ID NO: 37; the MFalpha2 signal peptide; and the HDEL signal peptide. In some embodiments, such a THCAS comprises the amino acid sequence shown below, in which signal peptides are underlined and bolded:

(SEQ ID NO: 233)
MKFISTFLTFILAAVSVTANPRENFLKCFSKHIPNNVA
NPKLVYTQHDQLYMSILNSTIQNLRFISDTTPKPLVIVTPSNNSHIQATI
LCSKKVGLQIRTRSGGHDAEGMSYISQVPFVVVDLRNMHSIKIDVHSQTA
WVEAGATLGEVYYWINEKNENLSFPGGYCPTVGVGGHFSGGGYGALMRNY
GLAADNIIDAHLVNVDGKVLDRKSMGEDLFWAIRGGGGENFGIIAAWKIR
LVAVPSRATIFSVKRNMEIHGLVKLFNKWQNIAYKYDKDLLLMTHFITRN
IIDNQGKNKTTVHGYFSCIFHGGVDSLVNLMNKSYPELGIKKTDCKEFSW
IDTTIFYSGVVNFNTANFKKEILLDRSAGKKTAFSIKLDYVKKPIPETAM
VKILEKLYEEDVGVGMYVLYPYGGIMEEISESAIPFPHRAGIMYELWYTA
SWEKQEDNEKHINWVRSVYNFTTPYVSQNPRLAYLNYRDLDLGKTNPESP
NNYTQARIWGEKYFGKNFNRLVKVKTKADPNNFFRNEQSIPPLPPHHH
HDEL.

A non-limiting example of a nucleic acid sequence encoding SEQ ID NO: 233 is shown below, in which sequences encoding signal peptides are underlined and bolded:

(SEQ ID NO: 226)
atgaagtttatcagtaccttcttgacctttatcttggccgc
tgtctccgtaaccgctaatcctagagaaaactttctgaaat
gcttctctaaacacattcccaataatgttgctaatcctaaactggtatat
acccaacacgatcaattgtacatgtctatcttgaacagcacgatacagaa
cttgagattcatatcagatacgacaccaaagcctttggttattgttaccc
ctagtaataatagtcatatccaggctacaatattgtgtagtaagaaagtg
ggcctacaaataagaaccagatcaggtggtcatgatgcggaaggcatgtc
ttatatttctcaggtaccatttgtggtggttgatttgagaaacatgcaca
gtatcaagatagacgttcacagtcaaacggcatgggggaggcgggtgcta
cattaggtgaagtttattactggatcaacgaaaagaatgaaaatctgtct
ttcccaggcggctactgtcctacagttggcgttggcgggcatttctctgg
cggtggttatggagccttgatgagaaactatggcttggccgcagataata
taatcgacgctcacttggttaacgttgatggtaaggtccttgatagaaaa
tccatgggtgaagatttgttttgggcaattagggggggtggtggtgaaaa
ttttggaattattgcggcctggaaaattaggttggtagcagttccttcca
gggcaaccattttttctgttaagagaaacatggaaattcatggcctggtg
aaattgtttaacaagtggcagaacatcgcatacaagtacgataaagattt
gttgttaatgacccactttattaccaggaacattattgataaccaaggta
aaaataagaccaccgtccatggatacttttcgtgcatatttcatggtggt
gttgatagtctagttaatttaatgaacaaatcttatcctgaactagggat
caaaaaaactgattgcaaggaattctcttggatagacacaacaatttttt
acagtggtgtcgtcaatttcaatacagcaaattttaagaaagaaatatta
ttagataggagtgcaggcaaaaagacggctttttctattaaactagatta
tgttaagaagccaattccagagacagcaatggtaaagatcctagagaaac
tatacgaagaggatgtcggagtcggaatgtacgttctttatccatatggg
ggtattatggaggagatatccgaaagcgcaatcccatttccccatagagc
cggaattatgtatgaattgtggtacaccgcttcatgggagaagcaggagg
ataacgaaaagcacataaattgggtaaggtcagtttacaattttacaaca
ccatacgtctcacagaatccgagattggcttatttgaactacagagactt
ggacctgggaaagacaaacccagagagcccaaacaattacactcaagcaa
ggatttggggtgaaaagtacttcggtaaaaatttcaataggctggtgaaa
gtcaagactaaggcggatccgaacaacttcttcaggaacgaacaatccat
tccaccacttcctccacatcaccaccatgatgaatta.

In some embodiments, a THCAS comprises the amino acid sequence shown below:

(SEQ ID NO: 43)
NPRENFLKCFSKHIPNNVANPKLVYTQHDQLYMSILNSTIQNLRFISDTT
PKPLVIVTPSNNSHIQATILCSKKVGLQIRTRSGGHDAEGMSYISQVPFV
VVDLRNMHSIKIDVHSQTAWVEAGATLGEVYYWINEKNENLSFPGGYCPT
VGVGGHFSGGGYGALMRNYGLAADNIIDAHLVNVDGKVLDRKSMGEDLFW
AIRGGGGENFGIIAAWKIKLVAVPSKSTIFSVKKNMEIHGLVKLFNKWQN
IAYKYDKDLVLMTHFITKNITDNHGKNKTTVHGYFSSIFHGGVDSLVDLM
NKSFPELGIKKTDCKEFSWIDTTIFYSGVVNFNTANFKKEILLDRSAGKK
TAFSIKLDYVKKPIPETAMVKILEKLYEEDVGAGMYVLYPYGGIMDEISE
SAIPFPHRAGIMYELWYTASWEKQEDNEKHLNWIRSVYNFTTPYVSQNPR
LAYLNYRDLDLGKTNPESPNNYTQARIWGEKYFGKNFNRLVKVKTKADPN
NFFRNEQSIPPLPPHHH.

A non-limiting example of a nucleic acid sequence encoding SEQ ID NO: 43 for expression in S. cerevisiae is:

(SEQ ID NO: 34)
aatccaagagaaaatttccttaaatgctttagcaagcatatccctaacaa
tgtcgcaaaccctaagcttgtgtacacgcaacatgatcagctttacatgt
cgatccttaacagcactatacaaaatttgaggtttatttcagatacgacc
ccgaaacctttagtcattgtgactccatccaacaatagccatatacaagc
aacaatattgtgtagtaagaaagtgggattgcaaatcaggaccagaagtg
gcggtcacgatgctgaaggaatgtcctacattagtcaagtccctttcgtt
gttgtagacttaaggaatatgcattcaattaaaatagacgttcattctca
gaccgcttgggtcgaagcaggagcaacacttggcgaggtatactactgga
taaatgagaaaaacgaaaatctttcattcccaggtggttactgccctact
gttggtgttggtggtcatttcagtggcggtggatatggagctttaatgag
gaattacggtcttgctgccgacaatattattgacgctcatttagtcaatg
tggacggaaaagtgttagataggaaaagcatgggtgaagatttattttgg
gccattagaggaggaggaggtgaaaattttggcattatagctgcatggaa
gattaaactagttgcagtcccctcaaaaagcactatcttttccgtaaaga
aaaatatggaaattcatggtctggttaagttgtttaataagtggcaaaac
atcgcttataagtatgataaagatcttgtgctaatgactcactttatcac
taaaaatataactgataatcatggtaaaaataaaacaacagttcatggtt
atttttctagtatctttcatggtggcgtggatagcttagttgaccttatg
aacaagtcgttcccagaactaggcattaagaagactgactgtaaagaatt
ttcatggatcgacacaacaatcttttattccggtgttgttaacttcaaca
ctgcaaattttaagaaagagatactattggatagatctgcgggtaaaaag
acggctttctccattaaactggactacgtaaagaaaccaatccctgagac
cgcaatggttaaaattttggaaaagttgtacgaagaagatgttggcgctg
gtatgtacgtcttatatccatatggtggaattatggacgaaatttctgag
tctgctattcccttccctcatagagccggcatcatgtatgagttatggta
cactgctagctgggagaaacaggaagataatgagaaacatttgaactgga
taaggagcgtctacaactttaccactccttacgtatcgcaaaatcctaga
ctagcctacttaaattatagagatctggatttggggaagactaaccctga
atctcccaataattacacacaagctagaatatggggcgagaaatacttcg
gaaaaaattttaacaggttagtaaaggttaaaaccaaggcggatcctaac
aatttcttccgtaatgaacaaagcattccacccttgccaccccaccatca
t.

In some embodiments, a THCAS comprises each of: SEQ ID NO: 43: the MFalpha2 signal peptide; and the HDEL signal peptide. In some embodiments, such a THCAS comprises the amino acid sequence shown below, in which signal peptides are underlined and bolded:

(SEQ ID NO: 234)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMDEISESAIPFPHRAGIMYELWYTASW
EKQEDNEKHLNWIRSVYNFTTPYVSQNPRLAYLNYRDLDL
GKTNPESPNNYTQARIWGEKYFGKNFNRLVKVKTKADPNN
FFRNEQSIPPLPPHHHHDEL

A non-limiting example of a nucleic acid sequence encoding SEQ ID NO: 234 is shown below, in which sequences encoding signal peptides are underlined and bolded:

(SEQ ID NO: 227)
atgaagtttatcagtaccttcttgacctttatcttggccg
ctgtctccgtaaccgctaatccaagagaaaatttccttaa
atgctttagcaagcatatccctaacaatgtcgcaaaccct
aagcttgtgtacacgcaacatgatcagctttacatgtcga
tccttaacagcactatacaaaatttgaggtttatttcaga
tacgaccccgaaacctttagtcattgtgactccatccaac
aatagccatatacaagcaacaatattgtgtagtaagaaag
tgggattgcaaatcaggaccagaagtggcggtcacgatgc
tgaaggaatgtcctacattagtcaagtccctttcgttgtt
gtagacttaaggaatatgcattcaattaaaatagacgttc
attctcagaccgcttgggtcgaagcaggagcaacacttgg
cgaggtatactactggataaatgagaaaaacgaaaatctt
tcattcccaggtggttactgccctactgttggtgttggtg
gtcatttcagtggcggtggatatggagctttaatgaggaa
ttacggtcttgctgccgacaatattattgacgctcattta
gtcaatgtggacggaaaagtgttagataggaaaagctggg
tgaagatttattttgggccattagaggaggaggaggtgaa
aattttggcattatagctgcatggaagattaaactagttg
cagtcccctcaaaaagcactatcttttccgtaaagaaaaa
tatggaaattcatggtctggttaagttgtttaataagtgg
caaaacatcgcttataagtatgataaagatcttgtgctaa
tgactcactttatcactaaaaatataactgataatcatgg
taaaaataaaacaacagttcatggttatttttctagtatc
tttcatggtggcgtggatagcttagttgaccttatgaaca
agtcgttcccagaactaggcattaagaagactgactgtaa
agaattttcatggatcgacacaacaatcttttattccggt
gttgttaacttcaacactgcaaattttaagaaagagatac
tattggatagatctgcgggtaaaaagacggctttctccat
taaactggactacgtaaagaaaccaatccctgagaccgca
atggttaaaattttggaaaagttgtacgaagaagatgttg
gcgctggtatgtacgtcttatatccatatggtggaattat
ggacgaaatttctgagtctgctattcccttccctcataga
gccggcatcatgtatgagttatggtacactgctagctggg
agaaacaggaagataatgagaaacatttgaactggataag
gagcgtctacaactttaccactccttacgtatcgcaaaat
cctagactagcctacttaaattatagagatctggatttgg
ggaagactaaccctgaatctcccaataattacacacaagc
tagaatatggggcgagaaatacttcggaaaaaattttaac
aggttagtaaaggttaaaaccaaggcggatcctaacaatt
tcttccgtaatgaacaaagcattccacccttgccacccca
ccatcatcatgatgaatta.

In some embodiments, a THCAS comprises the amino acid sequence shown

(SEQ ID NO: 40)
NPRENFLKCFSKHIPNNVANPKLVYTQHDQLYMSILNSTI
QNLRFISDTTPKPLVIVTPSNNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMDEISE
SAIPFPHRAGIMYELWYTASWEKQEDNEKHLNWIRNIYNF
MTPYVSKNPRLAYLNYRDLDIGINDPKNPNNYTQARIWGE
KYFGKNFDRLVKVKTLVDPNNFFRNEQSIPPLPRHRH.

A non-limiting example of a nucleic acid sequence encoding SEQ ID NO: 40 for expression in S. cerevisiae is:

(SEQ ID NO: 31)
aatcctagagaaaatttcttgaaatgcttttccaaacaca
tccctaacaatgtcgccaatcccaaattagtttatactca
acatgatcaactatatatgtcaatattaaattctactatt
caaaatttgagatttattagtgatactactccaaaacctc
tggtgattgtcactccaagcaacaattcccacatccaggc
aacaatcttgtgtagcaagaaagtgggacttcaaattagg
accagatctggtggccatgatgcggaaggtatgtcatata
ttagtcaagttccgtttgtagtagttgatttgcgtaatat
gcatagtatcaagatcgacgttcattcccaaacagcctgg
gtggaggcgggtgccactctgggggaagtttactactgga
taaatgagaagaacgagaatttatcatttccgggtggtta
ctgccctacagtaggggtaggtggtcatttttcaggcggt
ggctacggggcattaatgaggaattatggattggctgctg
ataacataatagacgcccatttagtcaacgtagatgggaa
agtgttggataggaagtctatgggtgaagacctattttgg
gcaatcagaggaggcggcggagagaacttcggtattattg
ccgcatggaaaataaagctagtcgccgtaccgtccaaatc
tactattttttccgtcaagaaaaatatggaaatccatggg
ctggttaaattgtttaataaatggcagaatatagcttata
agtacgataaggatctggttcttatgactcattttattac
caaaaatataacagacaatcatgggaaaaacaaaactact
gtacacggatatttctcaagtattttccatggcggggtag
atagcttagtagacttgatgaataaatcgttcccagaatt
aggaattaagaagactgactgtaaggaattcagttggata
gatacgaccattttctattcaggtgttgttaattttaaca
ccgccaactttaaaaaggaaattttactggacagatccgc
cggtaaaaagacagctttttcaatcaagttggattatgta
aaaaaacctataccagaaactgcaatggttaaaattcttg
aaaagttatacgaagaggatgtcggagccggaatgtacgt
attatacccttatgggggtatcatggatgaaatatctgaa
tcggctattccattccctcacagggcaggtattatgtatg
aattgtggtataccgctagctgggagaagcaagaggataa
cgaaaagcacctgaattggataaggaacatttataatttt
atgactccatatgtctcaaaaaacccacgtttagcttact
taaattatgtgatttggatataggtatcaacgacccaaag
aatccaaataactacacccaagctagaatttggggtgaga
aatactttggaaagaattttgataggctagtaaaagtgaa
gacacttgttgacccaaacaatttttttagaaacgaacaa
agcattccacctttgcctcgtcatagacac.

In some embodiments, a THCAS comprises each of: SEQ ID NO: 40; the MFalpha2 signal peptide; and the HDEL signal peptide. In some embodiments, such a THCAS comprises the amino acid sequence shown below, in which signal peptides are underlined and bolded:

(SEQ ID NO: 235)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLENKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMDEISESAIPFPHRAGIMYELWYTASW
EKQEDNEKHLNWIRNIYNFMTPYVSKNPRLAYLNYRDLDI
GINDPKNPNNYTQARIWGEKYFGKNFDRLVKVKTLVDPNN
FFRNEQSIPPLPRHRHHDEL

A non-limiting example of a nucleic acid sequence encoding SEQ ID NO: 235 is shown below, in which sequences encoding signal peptides are underlined and bolded:

(SEQ ID NO: 228)
atgaagtttatcagtaccttcttgacctttatcttggccg
ctgtctccgtaaccgctaatcctagagaaaatttcttgaa
atgcttttccaaacacatccctaacaatgtcgccaatccc
aaattagtttatactcaacatgatcaactatatatgtcaa
tattaaattctactattcaaaatttgagatttattagtga
tactactccaaaacctctggtgattgtcactccaagcaac
aattcccacatccaggcaacaatcttgtgtagcaagaaag
tgggacttcaaattaggaccagatctggtggccatgatgc
ggaaggtatgtcatatattagtcaagttccgtttgtagta
gttgatttgcgtaatatgcatagtatcaagatcgacgttc
attcccaaacagcctgggggaggcgggtgccactctgggg
gaagtttactactggataaatgagaagaacgagaatttat
catttccgggtggttactgccctacagtaggggtaggtgg
tcatttttcaggcggtggctacggggcattaatgaggaat
tatggattggctgctgataacataatagacgcccatttag
tcaacgtagatgggaaagtgttggataggaagtctatggg
tgaagacctattttgggcaatcagaggaggcggcggagag
aacttcggtattattgccgcatggaaaataaagctagtcg
ccgtaccgtccaaatctactattttttccgtcaagaaaaa
tatggaaatccatgggctggttaaattgtttaataaatgg
cagaatatagcttataagtacgataaggatctggttctta
tgactcattttattaccaaaaatataacagacaatcatgg
gaaaaacaaaactactgtacacggatatttctcaagtatt
ttccatggggggtagatagcttagtagacttgatgaataa
atcgttcccagaattaggaattaagaagactgactgtaag
gaattcagttggatagatacgaccattttctattcaggtg
ttgttaattttaacaccgccaactttaaaaaggaaatttt
actggacagatccgccggtaaaaagacagctttttcaatc
aagttggattatgtaaaaaaacctataccagaaactgcaa
tggttaaaattcttgaaaagttatacgaagaggatgtcgg
agccggaatgtacgtattatacccttatgggggtatcatg
gatgaaatatctgaatcggctattccattccctcacaggg
caggtattatgtatgaattgtggtataccgctagctggga
gaagcaagaggataacgaaaagcacctgaattggataagg
aacatttataattttatgactccatatgtctcaaaaaacc
cacgtttagcttacttaaattatcgtgatttggatatagg
tatcaacgacccaaagaatccaaataactacacccaagct
agaatttggggtgagaaatactttggaaagaattttgata
ggctagtaaaagtgaagacacttgttgacccaaacaattt
ttttagaaacgaacaaagcattccacctttgcctcgtcat
agacaccatgatgaatta.

In some embodiments, a THCAS comprises the amino acid sequence shown below:

(SEQ ID NO: 39)
NPQENFLKCFSEYIPNNPANPKFIYTQHDQLYMSVLNSTI
QNLRFTSDTTPKPLVIVTPSNVSHIQASILCSKKVGLQIR
TRSGGHDSEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLLLMTHFITRNITDNQGKNKTA
IHTYFSSVFLGGVDSLVDLMNKSFPELGIKKTDCKELSWI
DTTIFYSGVVNYNTTNFQKEILLDRSAGKKTAFSIKLDYV
KKPIPETAMVKILEKLYEEDVGVGMYVLYPYGGIMEEISE
SAIPFPHRAGIMYELWYTASWEKQEDNEKHINWVRSVYNF
TTPYVSQNPRLAYLNYRDLDLGKTNPESPNNYTQARIWGE
KYFGKNFNRLVKVKTKADPNNFFRNEQSIPPLPPHHH.

A non-limiting example of a nucleic acid sequence encoding SEQ ID NO: 39 for expression in S. cerevisiae is:

(SEQ ID NO: 30)
aatccccaagaaaatttcctgaaatgtttcagcgagtata
taccaaataaccctgcaaatcctaagtttatatatactca
acatgaccaactttacatgtctgttttaaattctaccatt
caaaatttgagatttacaagcgataccactccaaaacctt
tggttatagtgacaccgagtaacgttagtcatattcaggc
ttcaatcttgtgctcgaaaaaggtgggattacaaattagg
actcgtagcggaggtcatgattctgagggtatgagttaca
tttctcaagttcccttcgttgttgtggaccttaggaatat
gcattccataaaaatagatgtacactctcagacagcatgg
gttgaagctggtgccacactgggggaagtatattattgga
tcaacgaaaagaacgagaacctgtcgtttcctggtggcta
ttgcccaacagtcggtgtaggcggacactttagtggtggt
ggatacggtgccctaatgaggaactacggactagctgccg
ataatataatcgatgcacatctagtcaatgttgatggcaa
agtcttagacaggaagtctatgggtgaagacctattctgg
gctataagaggtggtggtggggaaaatttcggtatcatag
cggcatggaagataaaacttgtagctgtgcctagtaagtc
taccattttctctgtaaaaaaaaacatggagattcacggt
ttggtgaagctttttaataaatggcaaaacattgcctaca
aatacgataaagatttgctattgatgacacatttcataac
tagaaatattactgacaaccagggtaaaaacaagacggca
attcacacttacttttcttctgtttttttaggtggtgttg
attctttagttgatttgatgaataagagttttccggaact
gggcataaagaagaccgactgtaaggaattgtcttggatc
gacaccaccattttttactccggagttgttaattacaata
ctactaattttcaaaaagagatattattagatagatcagc
tggcaagaaaacagccttttctatcaaattggattatgta
aaaaaacccatacctgaaacagctatggtgaagattttag
aaaaattgtatgaagaagacgtgggcgtgggcatgtacgt
tctatacccctacggcggtattatggaagaaatttctgag
agcgcgatccctttcccgcatagggcaggaatcatgtatg
agttatggtacacggcatcgtgggaaaaacaagaagacaa
cgaaaagcatatcaactgggtgaggtctgtttacaatttt
acaactccctatgttagtcaaaatccaagattggcatact
taaattacagagatcttgacttgggaaaaacaaatccaga
atcgcccaacaattacacgcaagccagaatctggggggaa
aaatactttgggaaaaattttaacaggttagtcaaagtga
agactaaagccgatccaaataacttttttagaaatgagca
gtctatccctccattaccgccacatcatcat.

In some embodiments, a THCAS comprises each of: SEQ ID NO: 39; the MFalpha2 signal peptide; and the HDEL signal peptide. In some embodiments, such a THCAS comprises the amino acid sequence shown below, in which signal peptides are underlined and bolded:

(SEQ ID NO: 236)
MKFISTFLTFILAAVSVTANPQENFLKCFSEYIPNNPANP
KFIYTQHDQLYMSVLNSTIQNLRFTSDTTPKPLVIVTPSN
VSHIQASILCSKKVGLQIRTRSGGHDSEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLLL
MTHFITRNITDNQGKNKTAIHTYFSSVFLGGVDSLVDLMN
KSFPELGIKKTDCKELSWIDTTIFYSGVVNYNTTNFQKEI
LLDRSAGKKTAFSIKLDYVKKPIPETAMVKILEKLYEEDV
GVGMYVLYPYGGIMEEISESAIPFPHRAGIMYELWYTASW
EKQEDNEKHINWVRSVYNFTTPYVSQNPRLAYLNYRDLDL
GKTNPESPNNYTQARIWGEKYFGKNFNRLVKVKTKADPNN
FFRNEQSIPPLPPHHHHDEL

A non-limiting example of a nucleic acid sequence encoding SEQ ID NO: 236 is shown below, in which sequences encoding signal peptides are underlined and bolded:

(SEQ ID NO: 229)
atgaagtttatcagtaccttcttgacctttatcttggccg
ctgtctccgtaaccgctaatccccaagaaaatttcctgaa
atgtttcagcgagtatataccaaataaccctgcaaatcct
aagtttatatatactcaacatgaccaactttacatgtctg
ttttaaattctaccattcaaaatttgagatttacaagcga
taccactccaaaacctttggttatagtgacaccgagtaac
gttagtcatattcaggcttcaatcttgtgctcgaaaaagg
tgggattacaaattaggactcgtagcggaggtcatgattc
tgagggtatgagttacatttctcaagttcccttcgttgtt
gtggaccttaggaatatgcattccataaaaatagatgtac
actctcagacagcatgggttgaagctggtgccacactggg
ggaagtatattattggatcaacgaaaagaacgagaacctg
tcgtttcctggtggctattgcccaacagtcggtgtaggcg
gacactttagtggtggtggatacggtgccctaatgaggaa
ctacggactagctgccgataatataatcgatgcacatcta
gtcaatgttgatggcaaagtcttagacaggaagtctatgg
gtgaagacctattctgggctataagaggtggtggtgggga
aaatttcggtatcatagcggcatggaagataaaacttgta
gctgtgcctagtaagtctaccattttctctgtaaaaaaaa
acatggagattcacggtttggtgaagctttttaataaatg
gcaaaacattgcctacaaatacgataaagatttgctattg
atgacacatttcataactagaaatattactgacaaccagg
gtaaaaacaagacggcaattcacacttacttttcttctgt
ttttttaggtggtgttgattctttagttgatttgatgaat
aagagttttccggaactgggcataaagaagaccgactgta
aggaattgtcttggatcgacaccaccattttttactccgg
agttgttaattacaatactactaattttcaaaaagagata
ttattagatagatcagctggcaagaaaacagccttttcta
tcaaattggattatgtaaaaaaacccatacctgaaacagc
tatggtgaagattttagaaaaattgtatgaagaagacgtg
ggcgtgggcatgtacgttctatacccctacggcggtatta
tggaagaaatttctgagagcgcgatccctttcccgcatag
ggcaggaatcatgtatgagttatggtacacggcatcgtgg
gaaaaacaagaagacaacgaaaagcatatcaactgggtga
ggtctgtttacaattttacaactccctatgttagtcaaaa
tccaagattggcatacttaaattacagagatcttgacttg
ggaaaaacaaatccagaatcgcccaacaattacacgcaag
ccagaatctggggggaaaaatactttgggaaaaattttaa
caggttagtcaaagtgaagactaaagccgatccaaataac
ttttttagaaatgagcagtctatccctccattaccgccac
atcatcatcatgatgaatta.

In some embodiments, a THCAS comprises the amino acid sequence shown below:

(SEQ ID NO: 38)
NPRENFLKCFSKHIPNNVANPKLVYTQHDQLYMSILNSTI
QNLRFISDTTPKPLVIVTPSNNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASRNNYTQARIWGE
KYFGKNFNRLVKVKTKVDPNNFFRNEQSIPPLPPHHH

A non-limiting example of a nucleic acid sequence encoding SEQ ID NO: 38 for expression in S. cerevisiae is:

(SEQ ID NO: 29)
aatcccagagaaaattttttgaagtgtttttcaaagcaca
tacctaacaacgtcgccaatccgaagcttgtttatacaca
acacgatcaactatacatgtctattttgaatagtactatt
cagaatcttagatttatctcagacacaacacctaagccat
tggttattgtgacaccgtctaataattctcacattcaagc
aacaatattgtgctcgaaaaaggttggcttgcagatcaga
actagaagtggtggacatgatgcagaagggatgtcatata
tatctcaagtacccttcgtagttgttgatttaagaaatat
gcactctatcaaaatagatgtgcacagtcaaactgcttgg
gtcgaagctggcgcaactctaggggaggtgtactattgga
ttaatgaaaaaaacgaaaatctatcctttcctggcggtta
ctgcccgactgtaggtgtcggagggcacttcagtggtggt
ggatatggagctttgatgaggaactatggcttggctgcag
ataacattatagacgctcacctagtaaacgtagatgggaa
agtcttagatagaaaatcaatgggtgaggatttgttttgg
gctattcgtggggagggggcgaaaactttggtatcattgc
cgcatggaagataaaacttgtggctgttcccagcaaatca
actattttctctgttaagaagaatatggaaattcatgggt
tagttaaacttttcaataagtggcaaaatattgcctataa
atatgacaaagatttggtattgatgacacatttcatcact
aagaatatcactgacaatcatggaaagaacaaaacaacag
tacacggttacttctcctcaattttccatggtggtgtcga
ttcccttgtcgatctgatgaacaagtctttccctgaacta
ggtataaagaaaaccgactgtaaagagttttcatggatag
acacaacgatattttattcaggagtggtgaactttaacac
tgcaaatttcaaaaaggagattttattggacagatctgca
ggtaagaagactgcctttagcattaaattggactatgtaa
aaaagccgatcccagagaccgctatggttaaaatcttaga
aaagttatacgaggaagacgtcggtgcaggaatgtatgtc
ttgtatccttatggcggtatcatggaagaaatatcagagt
ctgctatcccatttccacatagagcagggataatgtacga
gttgtggtatactgcatcatgggaaaagcaagaggataac
gaaaaacacataaactgggtaagatccgtttataatttta
ctactccatacgtatctcaaaatcctagattagcttattt
aaactacagagatttagatttagggaaaaccaaccatgct
agtaggaacaactacacccaagccagaatatggggtgaaa
aatactttggaaaaaattttaataggttagtaaaagtgaa
aacaaaagtcgatcccaacaattttttcagaaacgagcaa
tccatacctcccttacctccgcaccatcac.

In some embodiments, a THCAS comprises each of: SEQ ID NO: 38; the MFalpha2 signal peptide; and the HDEL signal peptide. In some embodiments, such a THCAS comprises the amino acid sequence shown below, in which signal peptides are underlined and bolded:

(SEQ ID NO: 237)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASRNNYTQARIWGEKYFGKNFNRLVKVKTKVDPNN
FFRNEQSIPPLPPHHHHDEL

A non-limiting example of a nucleic acid sequence encoding SEQ ID NO: 237 is shown below, in which sequences encoding signal peptides are underlined and bolded:

(SEQ ID NO: 230)
atgaagtttatcagtaccttcttgacctttatcttggccg
ctgtctccgtaaccgctaatcccagagaaaattttttgaa
gtgtttttcaaagcacatacctaacaacgtcgccaatccg
aagcttgtttatacacaacacgatcaactatacatgtcta
ttttgaatagtactattcagaatcttagatttatctcaga
cacaacacctaagccattggttattgtgacaccgtctaat
aattctcacattcaagcaacaatattgtgctcgaaaaagg
ttggcttgcagatcagaactagaagtggtggacatgatgc
agaagggatgtcatatatatctcaagtacccttcgtagtt
gttgatttaagaaatatgcactctatcaaaatagatgtgc
acagtcaaactgcttgggtcgaagctggcgcaactctagg
ggaggtgtactattggattaatgaaaaaaacgaaaatcta
tcctttcctggcggttactgcccgactgtaggtgtcggag
ggcacttcagtggtggtggatatggagctttgatgaggaa
ctatggcttggctgcagataacattatagacgctcaccta
gtaaacgtagatgggaaagtcttagatagaaaatcaatgg
gtgaggatttgttttgggctattcgtggcggagggggcga
aaactttggtatcattgccgcatggaagataaaacttgtg
gctgttcccagcaaatcaactattttctctgttaagaaga
atatggaaattcatgggttagttaaacttttcaataagtg
gcaaaatattgcctataaatatgacaaagatttggtattg
atgacacatttcatcactaagaatatcactgacaatcatg
gaaagaacaaaacaacagtacacggttacttctcctcaat
tttccatggtggtgtcgattcccttgtcgatctgatgaac
aagtctttccctgaactaggtataaagaaaaccgactgta
aagagttttcatggatagacacaacgatattttattcagg
agtggtgaactttaacactgcaaatttcaaaaaggagatt
ttattggacagatctgcaggtaagaagactgcctttagca
ttaaattggactatgtaaaaaagccgatcccagagaccgc
tatggttaaaatcttagaaaagttatacgaggaagacgtc
ggtgcaggaatgtatgtcttgtatccttatggcggtatca
tggaagaaatatcagagtctgctatcccatttccacatag
agcagggataatgtacgagttgtggtatactgcatcatgg
gaaaagcaagaggataacgaaaaacacataaactgggtaa
gatccgtttataattttactactccatacgtatctcaaaa
tcctagattagcttatttaaactacagagatttagattta
gggaaaaccaaccatgctagtaggaacaactacacccaag
ccagaatatggggtgaaaaatactttggaaaaaattttaa
taggttagtaaaagtgaaaacaaaagtcgatcccaacaat
tttttcagaaacgagcaatccatacctcccttacctccgc
accatcaccatgatgaatta.

In some embodiments, a THCAS comprises the amino acid sequence shown below:

(SEQ ID NO: 42)
NPRENFLKCFSKHIPNNVANPKLVYTQHDQLYMSILNSTI
QNLRFISDTTPKPLVIVTPSNNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLLLMTHFITRNITDNQGKNKTA
IHTYFSSVFLGGVDSLVDLMNKSFPELGIKKTDCKELSWI
DTTIFYSGVVNFNTANFKKEILLDRSAGKKTAFSIKLDYV
KKPIPETAMVKILEKLYEEDVGAGMYVLYPYGGIMDEISE
SAIPFPHRAGIMYELWYTASWEKQEDNEKHINWVRSVYNF
TTPYVSQNPRLAYLNYRDLDLGKTNPESPNNYTQARIWGE
KYFGKNFNRLVKVKTKADPNNFFRNEQSIPPLPPHHH.

A non-limiting example of a nucleic acid sequence encoding SEQ ID NO: 42 for expression in S. cerevisiae is:

(SEQ ID NO: 33)
aatcccagggagaattttttaaaatgcttttcgaagcata
tcccaaacaatgtagctaatccaaagctggtttacacaca
acacgatcaactatatatgtctatattgaacagtaccatc
caaaacttaaggttcatatccgacacaactcctaaaccac
tagtaattgtgacgcctagcaacaattctcatatacaggc
aaccatattatgttcaaagaaagttggattgcaaatccgt
accagatcaggtggtcacgatgcagagggcatgagctata
tttctcaagtgccctttgtagtcgtggatcttagaaacat
gcatagtatcaagatcgatgtgcactcacaaacagcttgg
gttgaggcaggtgccactctgggggaagtatattattgga
taaatgaaaaaaatgaaaatttaagtttcccagggggtta
ctgtccgaccgttggagtaggtggacacttttccgggggg
ggttatggcgctttgatgagaaactatggtttggctgctg
acaacattatcgacgctcatcttgttaacgtagatggtaa
agtacttgatagaaaaagcatgggcgaagaccttttctgg
gccataagaggtggcggtggggagaattttgggattattg
ctgcctggaagattaaattggttgccgtcccatcaaagtc
cacaattttctcggttaagaaaaacatggagatccacggt
ttagttaagttatttaacaagtggcagaacattgcctaca
agtatgataaggatttgctacttatgacccactttattac
tagaaatatcactgacaatcaaggcaagaacaaaacagca
atacatacctactttagttcagtttttttaggtggagtag
atagtctagttgatttaatgaataaatcctttccagaatt
ggggattaaaaagaccgattgtaaagagttgtcctggatt
gatactacaattttttacagcggtgtagttaattttaata
cagccaatttcaaaaaggaaattttattggacagatccgc
agggaaaaaaacggccttttctattaaattagattatgtt
aaaaagcccattcccgaaacagctatggttaaaattttgg
aaaaactttacgaggaggacgtgggtgctggtatgtatgt
attatatccgtatggcggtattatggatgaaatttccgag
tcagcaattcccttcccacacagggctggaatcatgtatg
aattatggtatactgcttcttgggagaagcaggaagataa
cgagaaacacattaattgggtaaggtcggtttacaatttt
accactccctacgtatcgcaaaacccccgtttggcctatt
taaattatagagacttagacttaggaaaaacaaacccaga
atcgcctaacaattacacccaagcaaggatatggggagaa
aaatattttggtaagaatttcaatcgtttagttaaggtga
agactaaagccgatccaaataactttttcaggaatgaaca
atccatcccaccacttcctcctcatcatcat.

In some embodiments, a THCAS comprises each of: SEQ ID NO: 42; the MFalpha2 signal peptide; and the HDEL signal peptide. In some embodiments, such a THCAS comprises the amino acid sequence shown below, in which signal peptides are underlined and bolded:

(SEQ ID NO: 239)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLENKWQNIAYKYDKDLLL
MTHFITRNITDNQGKNKTAIHTYFSSVFLGGVDSLVDLMN
KSFPELGIKKTDCKELSWIDTTIFYSGVVNFNTANFKKEI
LLDRSAGKKTAFSIKLDYVKKPIPETAMVKILEKLYEEDV
GAGMYVLYPYGGIMDEISESAIPFPHRAGIMYELWYTASW
EKQEDNEKHINWVRSVYNFTTPYVSQNPRLAYLNYRDLDL
GKTNPESPNNYTQARIWGEKYFGKNFNRLVKVKTKADPNN
FFRNEQSIPPLPPHHHHDEL

A non-limiting example of a nucleic acid sequence encoding SEQ ID NO: 239 is shown below, in which sequences encoding signal peptides are underlined and bolded:

(SEQ ID NO: 232)
atgaagtttatcagtaccttcttgacctttatcttggccg
ctgtctccgtaaccgctaatcccagggagaattttttaaa
atgcttttcgaagcatatcccaaacaatgtagctaatcca
aagctggtttacacacaacacgatcaactatatatgtcta
tattgaacagtaccatccaaaacttaaggttcatatccga
cacaactcctaaaccactagtaattgtgacgcctagcaac
aattctcatatacaggcaaccatattatgttcaaagaaag
ttggattgcaaatccgtaccagatcaggtggtcacgatgc
agagggcatgagctatatttctcaagtgccctttgtagtc
gtggatcttagaaacatgcatagtatcaagatcgatgtgc
actcacaaacagcttgggttgaggcaggtgccactctggg
ggaagtatattattggataaatgaaaaaaatgaaaattta
agtttcccagggggttactgtccgaccgttggagtaggtg
gacacttttccggggggggttatggcgctttgatgagaaa
ctatggtttggctgctgacaacattatcgacgctcatctt
gttaacgtagatggtaaagtacttgatagaaaaagcatgg
gcgaagaccttttctgggccataagaggtggcggtgggga
gaattttgggattattgctgcctggaagattaaattggtt
gccgtcccatcaaagtccacaattttctcggttaagaaaa
acatggagatccacggtttagttaagttatttaacaagtg
gcagaacattgcctacaagtatgataaggatttgctactt
atgacccactttattactagaaatatcactgacaatcaag
gcaagaacaaaacagcaatacatacctactttagttcagt
ttttttaggtggagtagatagtctagttgatttaatgaat
aaatcctttccagaattggggattaaaaagaccgattgta
aagagttgtcctggattgatactacaattttttacagcgg
tgtagttaattttaatacagccaatttcaaaaaggaaatt
ttattggacagatccgcagggaaaaaaacggccttttcta
ttaaattagattatgttaaaaagcccattcccgaaacagc
tatggttaaaattttggaaaaactttacgaggaggacgtg
ggtgctggtatgtatgtattatatccgtatggcggtatta
tggatgaaatttccgagtcagcaattcccttcccacacag
ggctggaatcatgtatgaattatggtatactgcttcttgg
gagaagcaggaagataacgagaaacacattaattgggtaa
ggtcggtttacaattttaccactccctacgtatcgcaaaa
cccccgtttggcctatttaaattatagagacttagactta
ggaaaaacaaacccagaatcgcctaacaattacacccaag
caaggatatggggagaaaaatattttggtaagaatttcaa
tcgtttagttaaggtgaagactaaagccgatccaaataac
tttttcaggaatgaacaatccatcccaccacttcctcctc
atcatcatcatgatgaatta.

In some embodiments, a THCAS comprises the amino acid sequence shown

(SEQ ID NO: 141)
NPRENFLKCFSKHIPNNVANPKLVYTQHDQLYMSILNSTI
QNLRFISDTTPKPLVIVTPSNNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASPNNYTQARIWGE
KYFGKNFNRLVKVKTKVDPNNFFRNEQSIPPLPPHHH

A non-limiting example of a nucleic acid sequence encoding SEQ ID NO: 141 for expression in S. cerevisiae is:

(SEQ ID NO: 50)
aatcccagggagaactttcttaagtgtttcagtaaacata
tcccaaataacgtggcgaatccgaaattagtatacacgca
acacgatcagctatatatgagcattctgaatagtaccatt
caaaacttgcgctttatatcggacacaactccaaagcctt
tagtcatagttactccttcaaataattcccatattcaggc
tacaatcctctgctctaaaaaagttgggttgcaaatacgt
actagatcaggaggccacgatgccgaaggtatgtcttata
tctcccaagtgccattcgttgtcgtcgacttgaggaacat
gcattctattaagattgatgtacactcccagaccgcatgg
gttgaagctggtgccacattaggtgaagtatactattgga
taaacgagaaaaatgaaaatttatcgtttcctggaggcta
ttgtccaactgttggtgtcggtggtcatttctcaggcggg
ggctacggagcattgatgcgaaactacggactagcagctg
ataacattatagacgcccatctcgtgaatgtggatggtaa
agttcttgatagaaagagcatgggtgaagatttgttttgg
gcgatcagaggcggtggaggggaaaactttggtattattg
cggcttggaaaataaagctggtcgcagttccctcgaaaag
cacaatcttttctgtaaagaaaaatatggaaatacatggg
ttggtcaagttattcaataaatggcaaaacattgcttata
agtatgataaagacctcgttctgatgactcattttattac
gaaaaatattaccgacaatcaggtaagaataaaactacag
tgcacggttacttttcttcaattttccatggtggcgttga
ctctctagtagatttaatgaacaaaagtttccctgagtta
gggatcaagaaaacggattgtaaagaattttcttggatcg
acacaaccatattctattcgggtgttgttaactttaatac
cgcaaactttaaaaaggaaattttgttagatcgctctgct
ggaaagaaaacagcatttagtatcaaacttgactatgtaa
agaaaccgatacccgagactgccatggttaagatacttga
gaagctatacgaagaggatgtgggagctggcatgtacgtt
ctctacccttacggcggtataatggaagaaatttcagaat
ccgcaatcccattcccacacagagcaggtattatgtatga
actgtggtatactgcctcatgggagaaacaagaagataac
gaaaaacacattaattgggtccgtagcgtctacaatttca
ctacaccatatgtatcccaaaacccgagattggcttactt
gaattatagagacttggatctaggaaaaaccaatcatgcc
agcccgaataattatacacaggcaaggatttggggcgaga
agtatttcggcaagaacttcaacagattagtaaaagttaa
gaccaaagttgaccccaataacttttttagaaatgaacaa
agtatccctccactcccaccacatcatcat.

In some embodiments, a THCAS comprises each of: SEQ ID NO: 141; the MFalpha2 signal peptide; and the HDEL signal peptide. In some embodiments, such a THCAS comprises the amino acid sequence shown below, in which signal peptides are underlined and bolded:

(SEQ ID NO: 247)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASPNNYTQARIWGEKYFGKNFNRLVKVKTKVDPNN
FFRNEQSIPPLPPHHHHDEL

A non-limiting example of a nucleic acid sequence encoding SEQ ID NO: 247 is shown below, in which sequences encoding signal peptides are underlined and bolded:

(SEQ ID NO: 240)
atgaagtttatcagtaccttcttgacctttatcttggccg
ctgtctccgtaaccgctaatcccagggagaactttcttaa
gtgtttcagtaaacatatcccaaataacgtggcgaatccg
aaattagtatacacgcaacacgatcagctatatatgagca
ttctgaatagtaccattcaaaacttgcgctttatatcgga
cacaactccaaagcctttagtcatagttactccttcaaat
aattcccatattcaggctacaatcctctgctctaaaaaag
ttgggttgcaaatacgtactagatcaggaggccacgatgc
cgaaggtatgtcttatatctcccaagtgccattcgttgtc
gtcgacttgaggaacatgcattctattaagattgatgtac
actcccagaccgcatgggttgaagctggtgccacattagg
tgaagtatactattggataaacgagaaaaatgaaaattta
tcgtttcctggaggctattgtccaactgttggtgtcggtg
gtcatttctcaggcgggggctacggagcattgatgcgaaa
ctacggactagcagctgataacattatagacgcccatctc
gtgaatgtggatggtaaagttcttgatagaaagagcatgg
gtgaagatttgttttgggcgatcagaggcggtggagggga
aaactttggtattattgcggcttggaaaataaagctggtc
gcagttccctcgaaaagcacaatcttttctgtaaagaaaa
atatggaaatacatgggttggtcaagttattcaataaatg
gcaaaacattgcttataagtatgataaagacctcgttctg
atgactcattttattacgaaaaatattaccgacaatcacg
gtaagaataaaactacagtgcacggttacttttcttcaat
tttccatggtggcgttgactctctagtagatttaatgaac
aaaagtttccctgagttagggatcaagaaaacggattgta
aagaattttcttggatcgacacaaccatattctattcggg
tgttgttaactttaataccgcaaactttaaaaaggaaatt
ttgttagatcgctctgctggaaagaaaacagcatttagta
tcaaacttgactatgtaaagaaaccgatacccgagactgc
catggttaagatacttgagaagctatacgaagaggatgtg
ggagctggcatgtacgttctctacccttacggcggtataa
tggaagaaatttcagaatccgcaatcccattcccacacag
agcaggtattatgtatgaactgtggtatactgcctcatgg
gagaaacaagaagataacgaaaaacacattaattgggtcc
gtagcgtctacaatttcactacaccatatgtatcccaaaa
cccgagattggcttacttgaattatagagacttggatcta
ggaaaaaccaatcatgccagcccgaataattatacacagg
caaggatttggggcgagaagtatttcggcaagaacttcaa
cagattagtaaaagttaagaccaaagttgaccccaataac
ttttttagaaatgaacaaagtatccctccactcccaccac
atcatcatcatgatgaatta.

In some embodiments, a THCAS comprises the amino acid sequence shown below:

(SEQ ID NO: 144)
NPRENFLKCFSKHIPNNVANPKLVYTQHDQLYMSILNSTI
QNLRFISDTTPKPLVIVTPSDNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASPNNYTQARIWGE
KYFGKNFNRLVKVKTKVDPNNFFRNEQSIPPLPPHHH

A non-limiting example of a nucleic acid sequence encoding SEQ ID NO: 144 for expression in S. cerevisiae is:

(SEQ ID NO: 53)
aacccacgtgagaactttttgaaatgtttctctaagcata
tccctaacaatgtggctaacccaaagttagtttacacaca
gcacgatcaactatatatgagcattttgaactccactatc
caaaatctgaggttcattagtgacaccacgcccaaaccat
tggttattgtaaccccttctgataactctcatatccaagc
tactatattatgttccaagaaggtcggcttgcaaattaga
actagatcaggaggtcacgatgctgaaggtatgtcataca
tttcccaagttccattcgttgttgtagacctaagaaatat
gcactctatcaaaattgatgtccattctcaaacagcctgg
gtcgaagctggtgctaccttgggtgaagtttattactgga
ttaacgaaaagaatgaaaacttgtcgttcccaggtggtta
ctgtccaaccgtgggtgttggtggacactttagcggtggt
ggatacggcgccttgatgagaaactatggtttagctgctg
acaatatcatcgatgcacaccttgtcaacgttgatggtaa
ggtattggacagaaaatcaatgggtgaagacttgttctgg
gctataagaggtggcggtggtgagaactttggtatcatcg
ctgcatggaagattaagttagttgccgtcccatctaagtc
cactatcttcagtgttaaaaagaacatggaaattcatggt
ttggtcaagctatttaataaatggcagaacatcgcttaca
agtacgataaggacttagttttgatgacccattttataac
taaaaacattactgataaccacggtaaaaataagactaca
gttcacggttatttctcctctatcttccatggtggagttg
actctctggtcgacctaatgaacaagtccttcccagaatt
gggtattaagaagactgattgcaaagaattttcatggatc
gataccaccattttctactctggcgtggttaactttaata
cggctaactttaagaaggaaatattgttagaccgttcggc
cggtaagaaaaccgctttttctataaagttggattatgtt
aagaaacctattcctgaaacagccatggtcaagatcttag
aaaaattgtacgaagaggatgtaggagctggtatgtacgt
tctttacccatatggtggtattatggaagaaatatctgag
tctgccattccattcccacatagggcaggcattatgtacg
aattgtggtatactgctagctgggaaaagcaagaagacaa
tgaaaaacacataaattgggttagaagtgtatacaatttc
actaccccctacgtcagccaaaacccaagattggcctatc
taaactaccgtgacctggacctaggtaaaactaaccacgc
ttcaccaaacaactacacccaagctagaatctggggagag
aagtatttcggtaagaatttcaacagattggtcaaagtga
agaccaaggtcgatccaaataattttttcagaaacgagca
atctattcccccattaccaccacatcatcac.

In some embodiments, a THCAS comprises each of: SEQ ID NO: 144; the MFalpha2 signal peptide; and the HDEL signal peptide. In some embodiments, such a THCAS comprises the amino acid sequence shown below, in which signal peptides are underlined and bolded:

(SEQ ID NO: 248)
MKFISTFLTFILAAVSVTANPRENFLKCFSKHIPNNVANP
KLVYTQHDQLYMSILNSTIQNLRFISDTTPKPLVIVTPSD
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIAAWKIKLVA
VPSKSTIFSVKKNMEIHGLVKLFNKWONIAYKYDKDLVLM
THFITKNITDNHGKNKTTVHGYFSSIFHGGVDSLVDLMNK
SFPELGIKKTDCKEFSWIDTTIFYSGVVNFNTANFKKEIL
LDRSAGKKTAFSIKLDYVKKPIPETAMVKILEKLYEEDVG
AGMYVLYPYGGIMEEISESAIPFPHRAGIMYELWYTASWE
KQEDNEKHINWVRSVYNFTTPYVSQNPRLAYLNYRDLDLG
KTNHASPNNYTQARIWGEKYFGKNFNRLVKVKTKVDPNNF
FRNEQSIPPLPPHHHHDEL

A non-limiting example of a nucleic acid sequence encoding SEQ ID NO: 248 is shown below, in which sequences encoding signal peptides are underlined and bolded:

(SEQ ID NO: 241)
atgaagtttatcagtaccttcttgacctttatcttggccg
ctgtctccgtaaccgctaacccacgtgagaactttttgaa
atgtttctctaagcatatccctaacaatgtggctaaccca
aagttagtttacacacagcacgatcaactatatatgagca
ttttgaactccactatccaaaatctgaggttcattagtga
caccacgcccaaaccattggttattgtaaccccttctgat
aactctcatatccaagctactatattatgttccaagaagg
tcggcttgcaaattagaactagatcaggaggtcacgatgc
tgaaggtatgtcatacatttcccaagttccattcgttgtt
gtagacctaagaaatatgcactctatcaaaattgatgtcc
attctcaaacagcctgggtcgaagctggtgctaccttggg
tgaagtttattactggattaacgaaaagaatgaaaacttg
tcgttcccaggtggttactgtccaaccgtgggtgttggtg
gacactttagcggtggtggatacggcgccttgatgagaaa
ctatggtttagctgctgacaatatcatcgatgcacacctt
gtcaacgttgatggtaaggtattggacagaaaatcaatgg
gtgaagacttgttctgggctataagaggtggcggtggtga
gaactttggtatcatcgctgcatggaagattaagttagtt
gccgtcccatctaagtccactatcttcagtgttaaaaaga
acatggaaattcatggtttggtcaagctatttaataaatg
gcagaacatcgcttacaagtacgataaggacttagttttg
atgacccattttataactaaaaacattactgataaccacg
gtaaaaataagactacagttcacggttatttctcctctat
cttccatggtggagttgactctctggtcgacctaatgaac
aagtccttcccagaattgggtattaagaagactgattgca
aagaattttcatggatcgataccaccattttctactctgg
cgtggttaactttaatacggctaactttaagaaggaaata
ttgttagaccgttcggccggtaagaaaaccgctttttcta
taaagttggattatgttaagaaacctattcctgaaacagc
catggtcaagatcttagaaaaattgtacgaagaggatgta
ggagctggtatgtacgttctttacccatatggtggtatta
tggaagaaatatctgagtctgccattccattcccacatag
ggcaggcattatgtacgaattgtggtatactgctagctgg
gaaaagcaagaagacaatgaaaaacacataaattgggtta
gaagtgtatacaatttcactaccccctacgtcagccaaaa
cccaagattggcctatctaaactaccgtgacctggaccta
ggtaaaactaaccacgcttcaccaaacaactacacccaag
ctagaatctggggagagaagtatttcggtaagaatttcaa
cagattggtcaaagtgaagaccaaggtcgatccaaataat
tttttcagaaacgagcaatctattcccccattaccaccac
atcatcaccatgatgaatta

In some embodiments, a THCAS comprises the amino acid sequence shown below:

(SEQ ID NO: 155)
NPQENFLKCFSQYIPTNVTNAKLVYTQHDQFYMSILNSTI
QNLRFTSDTTPKPLVIITPLNVSHIQGTILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASPNNYTQARIWGE
KYFGKNFNRLVKVKTKVDPNNFFRNEQSIPPLPLRHH

A non-limiting example of a nucleic acid sequence encoding SEQ ID NO: 155 for expression in S. cerevisiae is:

(SEQ ID NO: 64)
aaccctcaggaaaatttcctgaagtgtttttctcaatata
ttccaactaatgtgacaaacgccaaactcgtttacacgca
acacgatcaattttatatgtccatacttaatagtaccatc
cagaacttgagattcacttcggacacaacaccgaaacccc
tagtcattataactcctttaaatgtaagccatattcaagg
taccatcctatgctcaaagaaagttgggttacagattagg
actcgttcaggaggtcatgatgctgagggcatgtcttaca
tcagtcaagtcccatttgttgtagtggatttgcgaaatat
gcattctataaaaattgacgttcacagtcaaacggcgtgg
gtagaagcaggagctacattaggtgaggtttattactgga
taaatgaaaagaacgaaaacttgagctttccaggcggata
ttgtcctactgtaggtgtgggcggacatttctctggtggt
gggtacggtgcattgatgagaaattatggcttagccgctg
ataatattattgatgcacacctggtcaatgttgacgggaa
agttcttgacagaaagtccatgggggaggatctcttctgg
gctatccgcggtggtggaggtgaaaattttggtattatcg
cagcctggaaaattaaactggtcgctgtcccatcaaagtc
gaccatattttctgttaagaaaaacatggaaattcatgga
ttggttaagctatttaataaatggcagaacattgcatata
agtatgataaagacttagtgctaatgacccatttcataac
taaaaacatcacagataaccacggtaagaataaaacaacc
gtgcatggctacttttcctcaatattccatggaggcgtag
atagtctggtagatcttatgaataagtcttttcccgagct
tgggatcaagaaaaccgactgcaaggaattttcctggatt
gatactacgattttctactcaggtgtagtcaatttcaaca
cggccaattttaaaaaagaaatactgttggacaggtcggc
gggaaagaaaaccgcttttagcatcaagttagactatgtt
aaaaaaccgataccagaaactgccatggttaaaatattgg
agaaattatatgaagaggatgtgggcgcaggtatgtatgt
gctatacccttacggtggaattatggaagaaatttccgaa
agtgctattccgtttcctcatagagctggaataatgtatg
aattgtggtacactgcgtcatgggaaaaacaagaagataa
cgagaagcacattaattgggtaaggagcgtttataatttt
acaacgccttacgtcagtcaaaacccaaggctggcatatt
taaattatcgggatttagacctcggcaaaacaaatcacgc
ctctccaaacaattacacacaagcgagaatatggggtgaa
aagtattttggaaagaatttcaaccgtctagttaaagtca
agactaaggttgatcctaataacttcttcagaaacgaaca
gagcatccccccattgcctttacgtcaccat

In some embodiments, a THCAS comprises each of: SEQ ID NO: 155; the MFalpha2 signal peptide; and the HDEL signal peptide. In some embodiments, such a THCAS comprises the amino acid sequence shown below, in which signal peptides are underlined and bolded:

(SEQ ID NO: 249)
MKFISTFLTFILAAVSVTANPQENFLKCFSQYIPTNVTNA
KLVYTQHDQFYMSILNSTIQNLRFTSDTTPKPLVIITPLN
VSHIQGTILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASPNNYTQARIWGEKYFGKNFNRLVKVKTKVDPNN
FFRNEQSIPPLPLRHHHDEL.

A non-limiting example of a nucleic acid sequence encoding SEQ ID NO: 249 is shown below, in which sequences encoding signal peptides are underlined and bolded:

(SEQ ID NO: 242)
atgaagtttatcagtaccttcttgacctttatcttggccg
ctgtctccgtaaccgctaaccctcaggaaaatttcctgaa
gtgtttttctcaatatattccaactaatgtgacaaacgcc
aaactcgtttacacgcaacacgatcaattttatatgtcca
tacttaatagtaccatccagaacttgagattcacttcgga
cacaacaccgaaacccctagtcattataactcctttaaat
gtaagccatattcaaggtaccatcctatgctcaaagaaag
ttgggttacagattaggactcgttcaggaggtcatgatgc
tgagggcatgtcttacatcagtcaagtcccatttgttgta
gtggatttgcgaaatatgcattctataaaaattgacgttc
acagtcaaacggcgtgggtagaagcaggagctacattagg
tgaggtttattactggataaatgaaaagaacgaaaacttg
agctttccaggcggatattgtcctactgtaggtgtgggcg
gacatttctctggtggtgggtacggtgcattgatgagaaa
ttatggcttagccgctgataatattattgatgcacacctg
gtcaatgttgacgggaaagttcttgacagaaagtccatgg
gggaggatctcttctgggctatccgcggtggtggaggtga
aaattttggtattatcgcagcctggaaaattaaactggtc
gctgtcccatcaaagtcgaccatattttctgttaagaaaa
acatggaaattcatggattggttaagctatttaataaatg
gcagaacattgcatataagtatgataaagacttagtgcta
atgacccatttcataactaaaaacatcacagataaccacg
gtaagaataaaacaaccgtgcatggctacttttcctcaat
attccatggaggcgtagatagtctggtagatcttatgaat
aagtcttttcccgagcttgggatcaagaaaaccgactgca
aggaattttcctggattgatactacgattttctactcagg
tgtagtcaatttcaacacggccaattttaaaaaagaaata
ctgttggacaggtcggcgggaaagaaaaccgcttttagca
tcaagttagactatgttaaaaaaccgataccagaaactgc
catggttaaaatattggagaaattatatgaagaggatgtg
ggcgcaggtatgtatgtgctatacccttacggtggaatta
tggaagaaatttccgaaagtgctattccgtttcctcatag
agctggaataatgtatgaattgtggtacactgcgtcatgg
gaaaaacaagaagataacgagaagcacattaattgggtaa
ggagcgtttataattttacaacgccttacgtcagtcaaaa
cccaaggctggcatatttaaattatcgggatttagacctc
ggcaaaacaaatcacgcctctccaaacaattacacacaag
cgagaatatggggtgaaaagtattttggaaagaatttcaa
ccgtctagttaaagtcaagactaaggttgatcctaataac
ttcttcagaaacgaacagagcatccccccattgcctttac
gtcaccatcatgatgaatta.

In some embodiments, a THCAS comprises the amino acid sequence shown below:

(SEQ ID NO: 158)
NPRENFLKCFSKHIPNNVANPKLVYTQHDQLYMSILNSTIQ
NLRFISDTTPKPLVIVTPSNNSHIQATILCSKKVGLQIRT
RSGGHDAEGMSYISQVPFVVVDLRNMHSIKIDVHSQTAWV
EAGATLGEVYYWINEKNENLSFPGGYCPTVGVGGHFSGGG
YGALMRNYGLAADNIIDAHLVNVDGKVLDRKSMGEDLFWA
IRGGGGENFGIIAAWKIKLVAVPSKSTIFSVKKNMEIHGL
VKLFNKWQNIAYKYDKDLVLMTHFITKNITDNHGKNKTTV
HGYFSSIFHGGVDSLVDLMNKSFPELGIKKTDCKEFSWID
TTIFYSGVVNFNTANFKKEILLDRSAGKKTAFSIKLDYVK
KPIPETAMVKILEKLYEEDVGAGMYVLYPYGGIMEEISES
AIPFPHRAGIMYELWYTASWEKQEDNEKHINWVRSVYNFT
TPYVSQNPRLAYLNYRDLDLGKTNHASPNNYTQARIWGEK
YFGKNFNRLVKVKTKVDPNNFFRNEQSIPPLPPHHH.

A non-limiting example of a nucleic acid sequence encoding SEQ ID NO: 158 for expression in S. cerevisiae is:

(SEQ ID NO: 67)
aatcctcgagagaactttctgaagtgtttcagcaaacaca
taccaaataatgtagctaacccgaaattggtttacacaca
gcatgatcaactatatatgagtatcttaaattctacgatt
caaaacttgaggtttatttccgacaccactccaaagcctc
ttgtcattgtgactccctcaaacaattcacatatccaagc
gaccatattatgtctaaaaaagttggtttacagatcagaa
cacgttcgggagggcatgatgccgaaggtatgtcctatat
tagtcaagtgccattcgtagttgtcgatctcagaaacatg
cacagcattaagatcgacgtccattctcaaactgcatggg
ttgaagccggcgcaacattgggtgaggtttactattggat
aaatgaaaaaaatgaaaacctctcgtttcccggaggctat
tgtcctacggtaggtgttgggggtcacttctcaggtggag
gctacggcgctctaatgagaaattacggtcttgctgcgga
taatattatagacgcacatctagtgaacgtcgatggcaag
gtgttagatcgcaaatctatgggggaagatttgttttggg
ctatcaggggtggtggaggtgagaatttcggcattattgc
agcatggaagattaaactggtcgccgttccaagtaagtct
actatattttccgtaaaaaaaaatatggaaattcatggac
tggtaaagttgtttaacaaatggcagaacatcgcttataa
atatgataaggacttagttttgatgacccacttcattaca
aagaacataactgataatcatggtaaaaataaaaccactg
tacacggttacttttcctcaatttttcatggaggagtgga
ttcacttgttgacctgatgaacaagagtttccagaattgg
gcatcaaaaaaacagactgcaaggaattttttggatagat
accacaatcttctactcaggtgtcgtgaattttaacactg
ctaattttaaaaaggagattttactagatagatccgcggg
gaagaaaacagcattttcaataaagcttgattatgtaaaa
aaacccattccggaaaccgctatggttaaaatattagaga
agttatatgaagaagatgtcggtgccggaatgtacgttct
ctatccttatggcgggatcatggaggaaatatcggagagc
gctattccattcccccaccgtgccggtattatgtacgaac
tatggtacacagcatcttgggaaaaacaagaggataatga
aaaacatattaattgggtgcggtctgtttataattttacg
accccatatgtgtcacaaaacccaaggttagcctacttga
attatagagacttggacctaggcaagacgaaccacgcttc
tcctaataattatactcaggctcgtatatggggtgaaaag
tacttcggtaagaacttcaatagactggtgaaggtgaaaa
ctaaagttgacccaaacaatttctttagaaatgaacagtc
aatcccccctcttcctcctcatcatcat.

In some embodiments, a THCAS comprises each of SEQ ID NO: 158; the MFalpha2 signal peptide; and the HDEL signal peptide. In some embodiments, such a THCAS comprises the amino acid sequence shown below, in which signal peptides are underlined and bolded:

(SEQ ID NO: 250)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASPNNYTQARIWGEKYFGKNFNRLVKVKTKVDPNN
FFRNEQSIPPLPPHHHHDEL.

A non-limiting example of a nucleic acid sequence encoding SEQ ID NO: 250 is shown below, in which sequences encoding signal peptides are underlined and bolded:

(SEQ ID NO: 243)
atgaagtttatcagtaccttcttgacctttatcttggccg
ctgtctccgtaaccgctaatcctcgagagaactttctgaa
gtgtttcagcaaacacataccaaataatgtagctaacccg
aaattggtttacacacagcatgatcaactatatatgagta
tcttaaattctacgattcaaaacttgaggtttatttccga
caccactccaaagcctcttgtcattgtgactccctcaaac
aattcacatatccaagcgaccatattatgctctaaaaaag
ttggtttacagatcagaacacgttcgggagggcatgatgc
cgaaggtatgtcctatattagtcaagtgccattcgtagtt
gtcgatctcagaaacatgcacagcattaagatcgacgtcc
attctcaaactgcatgggttgaagccggcgcaacattggg
tgaggtttactattggataaatgaaaaaaatgaaaacctc
tcgtttcccggaggctattgtcctacggtaggtgttgggg
gtcacttctcaggtggaggctacggcgctctaatgagaaa
ttacggtcttgctgcggataatattatagacgcacatcta
gtgaacgtcgatggcaaggtgttagatcgcaaatctatgg
gggaagatttgttttgggctatcaggggtggtggaggtga
gaatttcggcattattgcagcatggaagattaaactggtc
gccgttccaagtaagtctactatattttccgtaaaaaaaa
atatggaaattcatggactggtaaagttgtttaacaaatg
gcagaacatcgcttataaatatgataaggacttagttttg
atgacccacttcattacaaagaacataactgataatcatg
gtaaaaataaaaccactgtacacggttacttttcctcaat
ttttcatggaggagtggattcacttgttgacctgatgaac
aagagtttcccagaattgggcatcaaaaaaacagactgca
aggaattttcttggatagataccacaatcttctactcagg
tgtcgtgaattttaacactgctaattttaaaaaggagatt
ttactagatagatccgcggggaagaaaacagcattttcaa
taaagcttgattatgtaaaaaaacccattccggaaaccgc
tatggttaaaatattagagaagttatatgaagaagatgtc
ggtgccggaatgtacgttctctatccttatggcgggatca
tggaggaaatatcggagagcgctattccattcccccaccg
tgccggtattatgtacgaactatggtacacagcatcttgg
gaaaaacaagaggataatgaaaaacatattaattgggtgc
ggtctgtttataattttacgaccccatatgtgtcacaaaa
cccaaggttagcctacttgaattatagagacttggaccta
ggcaagacgaaccacgcttctcctaataattatactcagg
ctcgtatatggggtgaaaagtacttcggtaagaacttcaa
tagactggtgaaggtgaaaactaaagttgacccaaacaat
ttctttagaaatgaacagtcaatcccccctcttcctcctc
atcatcatcatgatgaatta

In some embodiments, a THCAS comprises the amino acid sequence shown below:

(SEQ ID NO: 198)
NPQENFLKCFSQYIPTNVTNAKLVYTQHDQFYMSILNSTI
QNLRFTSDTTPKPLVIITPLNVSHIQGTILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVAVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNFNTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASPNNYTQARIWGE
KYFGKNFNRLVKVKTKVDPNNFFRNEQSIPPLPLRHH

A non-limiting example of a nucleic acid sequence encoding SEQ ID NO: 198 for expression in S. cerevisiae is:

(SEQ ID NO: 107)
aatccacaagagaactttcttaaatgtttctctcagtaca
tcccgacgaatgtcactaacgcgaagttagtttataccca
acatgaccagttctatatgagtatactgaatagcacaatt
caaaacttgcgttttacatcggatactactcctaaaccat
tggtaattatcacccctctcaatgtgtcccacatacaagg
gacaattctatgctctaaaaaggttggtttacaaatccga
acgaggtcaggcggacatgatgcagaaggtatgtcataca
tttcccaagtaccctttgttgtggtcgatttaagaaatat
gcattctataaaaattgacgttcacagtcagacagcctgg
gtagaagctggtgctaccttgggagaagtctattactgga
ttaacgagaagaatgaaaatttaagcttcccaggcggtta
ttgtcccactgttggagtcggtggccactttagcgggggt
ggttatggagcactaatgagaaactacggcctggccgctg
ataacataatcgacgcacatcttgttaatgtagatggtaa
agtactagatcgcaagagtatgggagaagatctattttgg
gccattcgtgggggtggtggagagaatttcggcataattg
ctgcatggaaaataaagcttgttgcggtgccttcaaaatc
cactatcttttctgttaaaaagaacatggaaattcatggc
ttggtcaaattattcaataagtggcaaaatatcgcttata
agtacgacaaagatttggtgctgatgacacactttataac
taagaacattacagacaatcatggtaaaaacaaaaccacc
gtgcacgggtacttttcttcaatttttcatggcggtgtcg
attcgctggtagacttgatgaataaaagcttcccggagtt
aggtattaaaaagactgattgtaaagaattttcttggata
gatactacaattttctattccggagttgtaaactttaata
ctgccaatttcaaaaaagaaatcttacttgacagatctgc
tgggaaaaagacggcattttctataaaattggattacgtt
aagaaaccaatacccgaaaccgcaatggttaaaatcctgg
agaagttatatgaagaagatgtgggagctggtatgtatgt
actatacccatacggtggcataatggaagagatctcagaa
tccgcaatccctttcccacatcgggctggtatcatgtatg
aattatggtatacagccagttgggaaaagcaagaagataa
cgaaaagcatattaactgggtgagatcggtctacaatttt
accactccctatgttagtcaaaaccctagactagcctatt
tgaattatagggacttagatctcggaaaaacgaaccacgc
atcacctaataactacacccaggcgagaatttggggtgag
aagtacttcggtaaaaattttaataggttggtcaaagtta
aaacaaaggttgatcccaacaacttctttcgcaatgagca
atcgattccaccattaccgttgcgacatcat.

In some embodiments, a THCAS comprises each of: SEQ ID NO: 198; the MFalpha2 signal peptide; and the HDEL signal peptide. In some embodiments, such a THCAS comprises the amino acid sequence shown below, in which signal peptides are underlined and bolded:

(SEQ ID NO: 251)
MKFISTFLTFILAAVSVTANPQENFLKCFSQYIPTNVTNA
KLVYTQHDQFYMSILNSTIQNLRFTSDTTPKPLVIITPLN
VSHIQGTILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
AVPSKSTIFSVKKNMEIHGLVKLFNKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASPNNYTQARIWGEKYFGKNFNRLVKVKTKVDPNN
FFRNEQSIPPLPLRHHHDEL

A non-limiting example of a nucleic acid sequence encoding SEQ ID NO: 251 is shown below, in which sequences encoding signal peptides are underlined and bolded:

(SEQ ID NO: 244)
atgaagtttatcagtaccttcttgacctttatcttggccg
ctgtctccgtaaccgctaatccacaagagaactttcttaa
atgtttctctcagtacatcccgacgaatgtcactaacgcg
aagttagtttatacccaacatgaccagttctatatgagta
tactgaatagcacaattcaaaacttgcgttttacatcgga
tactactcctaaaccattggtaattatcacccctctcaat
gtgtcccacatacaagggacaattctatgctctaaaaagg
ttggtttacaaatccgaacgaggtcaggcggacatgatgc
agaaggtatgtcatacatttcccaagtaccctttgttgtg
gtcgatttaagaaatatgcattctataaaaattgacgttc
acagtcagacagcctgggtagaagctggtgctaccttggg
agaagtctattactggattaacgagaagaatgaaaattta
agcttcccaggcggttattgtcccactgttggagtcggtg
gccactttagcgggggtggttatggagcactaatgagaaa
ctacggcctggccgctgataacataatcgacgcacatctt
gttaatgtagatggtaaagtactagatcgcaagagtatgg
gagaagatctattttgggccattcgtgggggtggtggaga
gaatttcggcataattgctgcatggaaaataaagcttgtt
gcggtgccttcaaaatccactatcttttctgttaaaaaga
acatggaaattcatggcttggtcaaattattcaataagtg
gcaaaatatcgcttataagtacgacaaagatttggtgctg
atgacacactttataactaagaacattacagacaatcatg
gtaaaaacaaaaccaccgtgcacgggtacttttcttcaat
ttttcatggcggtgtcgattcgctggtagacttgatgaat
aaaagcttcccggagttaggtattaaaaagactgattgta
aagaattttcttggatagatactacaattttctattccgg
agttgtaaactttaatactgccaatttcaaaaaagaaatc
ttacttgacagatctgctgggaaaaagacggcattttcta
taaaattggattacgttaagaaaccaatacccgaaaccgc
aatggttaaaatcctggagaagttatatgaagaagatgtg
ggagctggtatgtatgtactatacccatacggtggcataa
tggaagagatctcagaatccgcaatccctttcccacatcg
ggctggtatcatgtatgaattatggtatacagccagttgg
gaaaagcaagaagataacgaaaagcatattaactgggtga
gatcggtctacaattttaccactccctatgttagtcaaaa
ccctagactagcctatttgaattatagggacttagatctc
ggaaaaacgaaccacgcatcacctaataactacacccagg
cgagaatttggggtgagaagtacttcggtaaaaattttaa
taggttggtcaaagttaaaacaaaggttgatcccaacaac
ttctttcgcaatgagcaatcgattccaccattaccgttgc
gacatcatcatgatgaatta.

In some embodiments, a THCAS comprises the amino acid sequence shown below:

(SEQ ID NO: 200)
ANPRENFLKCFSKHIPNNVANPKLVYTQHDQLYMSILNST
IQNLRFISDTTPKPLVIVTPSNNSHIQATILCSKKVGLQI
RTRSGGHDAEGMSYISQVPFVVVDLRNMHSIKIDVHSQTA
WVEAGATLGEVYYWINEKNENLSFPGGYCPTVGVGGHFSG
GGYGALMRNYGLAADNIIDAHLVNVDGKVLDRKSMGEDLF
WAIRGGGGENFGIIAAWKIKLVAVPSKSTIFSVKKNMEIH
GLVKLFNKWQNIAYKYDKDLVLMTHFITKNITDNHGKNKT
TVHGYFSSIFHGGVDSLVDLMNKSFPELGIKKTDCKEFSW
IDTTIFYSGVVNFNTANFKKEILLDRSAGKKTAFSIKLDY
VKKPIPETAMVKILEKLYEEDVGAGMYVLYPYGGIMEEIS
ESAIPFPHRAGIMYELWYTASWEKQEDNEKHINWVRSVYN
FTTPYVSQNPRLAYLNYRDLDLGKTNHASPNNYTQARIWG
EKYFGKNFNRLVKVKTKVDPNNFFRNEQSIPPLPRHRH

A non-limiting example of a nucleic acid sequence encoding SEQ ID NO: 200 for expression in S. cerevisiae is:

(SEQ ID NO: 109)
gccaatccccgtgaaaacttcttaaaatgcttttctaagc
acatcccaaataacgtagcaaatcctaaattggtgtatac
acaacatgaccagctttacatgtcaattctaaacagtacg
atacaaaatctgaggtttatttcggataccactccaaagc
cgctcgttatagtcactccatccaataactcacatatcca
ggcgacaattttgtgttctaaaaaagttggtttacaaatt
agaacccggagcggagggcatgatgctgagggcatgtcct
atatctcccaagtccctttcgtagtggttgaccttagaaa
tatgcacagtataaagattgatgtccatagtcagactgcc
tgggttgaagctggtgcaacattaggtgaagtatactatt
ggataaacgaaaagaatgagaacttgtcatttccaggagg
ctactgtcctactgttggagtcggtgggcacttctctggt
ggaggctatggtgctctaatgagaaattacggtttagcag
cggataacattatagacgcccatctggtgaatgttgatgg
taaagttttggaccgaaagtctatgggtgaagatttattt
tgggctattcgtggaggagggggcgagaacttcggaatca
ttgcagcttggaaaataaagttggttgcagtacccagcaa
atcgaccatcttttctgtcaaaaaaaatatggaaattcac
ggcctcgtgaaactttttaataagtggcaaaatattgcgt
ataagtatgataaagatttagttctcatgacacattttat
caccaaaaacatcacggataatcatggtaaaaataagact
acggtccacgggtactttagttcaattttccatggtggtg
ttgattcacttgtagacctaatgaataagtcgttccctga
gttggggataaagaaaacagattgcaaagaattttcctgg
attgacactacgattttctactctggtgttgtcaacttta
acacagctaatttcaaaaaagaaattttgctagataggtc
tgctggcaaaaagacagctttttcaattaaactggactat
gtgaaaaaaccgatcccagaaactgccatggtaaagatat
tagaaaagctgtacgaggaagatgtaggtgcgggtatgta
tgtactatacccttatggcggtataatggaggaaatcagt
gaaagcgcaataccatttccccaccgcgccggcatcatgt
acgagctttggtataccgccagttgggaaaaacaagaaga
taatgagaagcatatcaactgggttagatcagtttataat
ttcactaccccctacgtgtcgcaaaacccacggttggcct
atctaaattatagagacttagatttgggtaaaacaaatca
tgcttcaccaaataactacactcaggctaggatatggggc
gaaaaatacttcggtaagaactttaatagattagttaaag
tcaagacgaaggttgatccgaataatttttttagaaacga
gcaatccattcctcccttaccgagacacagacat.

In some embodiments, a THCAS comprises each of: SEQ ID NO: 200; the MFalpha2 signal peptide; and the HDEL signal peptide. In some embodiments, such a THCAS comprises the amino acid sequence shown below, in which signal peptides are underlined and bolded:

(SEQ ID NO: 252)
MKFISTFLTFILAAVSVTAANPRENFLKCFSKHIPNNVAN
PKLVYTQHDQLYMSILNSTIQNLRFISDTTPKPLVIVTPS
NNSHIQATILCSKKVGLQIRTRSGGHDAEGMSYISQVPFV
VVDLRNMHSIKIDVHSQTAWVEAGATLGEVYYWINEKNEN
LSFPGGYCPTVGVGGHFSGGGYGALMRNYGLAADNIIDAH
LVNVDGKVLDRKSMGEDLFWAIRGGGGENFGIIAAWKIKL
VAVPSKSTIFSVKKNMEIHGLVKLFNKWQNIAYKYDKDLV
LMTHFITKNITDNHGKNKTTVHGYFSSIFHGGVDSLVDLM
NKSFPELGIKKTDCKEFSWIDTTIFYSGVVNFNTANFKKE
ILLDRSAGKKTAFSIKLDYVKKPIPETAMVKILEKLYEED
VGAGMYVLYPYGGIMEEISESAIPFPHRAGIMYELWYTAS
WEKQEDNEKHINWVRSVYNFTTPYVSQNPRLAYLNYRDLD
LGKTNHASPNNYTQARIWGEKYFGKNFNRLVKVKTKVDPN
NFFRNEQSIPPLPRHRHHDEL

A non-limiting example of a nucleic acid sequence encoding SEQ ID NO: 252 is shown below, in which sequences encoding signal peptides are underlined and bolded:

(SEQ ID NO: 245)
atgaagtttatcagtaccttcttgacctttatcttggccg
ctgtctccgtaaccgctgccaatccccgtgaaaacttctt
aaaatgcttttctaagcacatcccaaataacgtagcaaat
cctaaattggtgtatacacaacatgaccagctttacatgt
caattctaaacagtacgatacaaaatctgaggtttatttc
ggataccactccaaagccgctcgttatagtcactccatcc
aataactcacatatccaggcgacaattttgtgttctaaaa
aagttggtttacaaattagaacccggagcggagggcatga
tgctgagggcatgtcctatatctcccaagtccctttcgta
gtggttgaccttagaaatatgcacagtataaagattgatg
tccatagtcagactgcctgggttgaagctggtgcaacatt
aggtgaagtatactattggataaacgaaaagaatgagaac
ttgtcatttccaggaggctactgtcctactgttggagtcg
gtgggcacttctctggtggaggctatggtgctctaatgag
aaattacggtttagcagcggataacattatagacgcccat
ctggtgaatgttgatggtaaagttttggaccgaaagtcta
tgggtgaagatttattttgggctattcgtggaggaggggg
cgagaacttcggaatcattgcagcttggaaaataaagttg
gttgcagtacccagcaaatcgaccatcttttctgtcaaaa
aaaatatggaaattcacggcctcgtgaaactttttaataa
gtggcaaaatattgcgtataagtatgataaagatttagtt
ctcatgacacattttatcaccaaaaacatcacggataatc
atggtaaaaataagactacggtccacgggtactttagttc
aattttccatggtggtgttgattcacttgtagacctaatg
aataagtcgttccctgagttggggataaagaaaacagatt
gcaaagaattttcctggattgacactacgattttctactc
tggtgttgtcaactttaacacagctaatttcaaaaaagaa
attttgctagataggtctgctggcaaaaagacagcttttt
caattaaactggactatgtgaaaaaaccgatcccagaaac
tgccatggtaaagatattagaaaagctgtacgaggaagat
gtaggtgcgggtatgtatgtactatacccttatggcggta
taatggaggaaatcagtgaaagcgcaataccatttcccca
ccgcgccggcatcatgtacgagctttggtataccgccagt
tgggaaaaacaagaagataatgagaagcatatcaactggg
ttagatcagtttataatttcactaccccctacgtgtcgca
aaacccacggttggcctatctaaattatagagacttagat
ttgggtaaaacaaatcatgcttcaccaaataactacactc
aggctaggatatggggcgaaaaatacttggtaagaacttt
aatagattagttaaagtcaagacgaaggttgatccgaata
atttttttagaaacgagcaatccattcctcccttaccgag
acacagacatcatgatgaatta.

In some embodiments, a THCAS comprises the amino acid sequence shown below:

(SEQ ID NO: 203)
NPRENFLKCFSKHIPNNVANPKLVYTQHDQLYMSILNSTI
QNLRFISDTTPKPLVIVTPSNNSHIQATILCSKKVGLQIR
TRSGGHDAEGMSYISQVPFVVVDLRNMHSIKIDVHSQTAW
VEAGATLGEVYYWINEKNENLSFPGGYCPTVGVGGHFSGG
GYGALMRNYGLAADNIIDAHLVNVDGKVLDRKSMGEDLFW
AIRGGGGENFGIIAAWKIKLVDVPSKSTIFSVKKNMEIHG
LVKLFNKWQNIAYKYDKDLVLMTHFITKNITDNHGKNKTT
VHGYFSSIFHGGVDSLVDLMNKSFPELGIKKTDCKEFSWI
DTTIFYSGVVNENTANFKKEILLDRSAGKKTAFSIKLDYV
KKPIPETAMVKILEKLYEEDVGAGMYVLYPYGGIMEEISE
SAIPFPHRAGIMYELWYTASWEKQEDNEKHINWVRSVYNF
TTPYVSQNPRLAYLNYRDLDLGKTNHASPNNYTQARIWGE
KYFGKNFNRLVKVKTKVDPNNFFRNEQSIPPLPPHHH.

A non-limiting example of a nucleic acid sequence encoding SEQ ID NO: 203 for expression in S. cerevisiae is:

(SEQ ID NO: 112)
aatcctcgtgagaactttctcaaatgcttctccaagcata
tacccaataacgttgcaaatccgaaattggtatatacgca
gcacgaccaactgtacatgtcaatcttaaatagtactatt
caaaaccttcgatttatttcggataccacaccaaagcctt
tggtgatagttactccatctaacaattctcatatccaagc
gacaattttatgtagcaaaaaggtcggcctacagattaga
acccgcagtgggggtcacgatgccgaaggtatgtcctata
tatctcaagtaccattcgtcgtggttgacttaaggaacat
gcattcaattaaaatcgatgtgcactcgcaaactgcttgg
gttgaagctggagcaacgttaggtgaggtatactattgga
ttaatgaaaagaatgaaaatttgtcttttccgggaggtta
ctgtcccaccgttggcgtgggcggtcatttttcaggggga
gggtatggagccctcatgagaaactacggtctagcagctg
ataatataatagacgctcatcttgtcaatgttgatggtaa
agtcctggacagaaaaagcatgggtgaggatctattctgg
gcgatcaggggtggcgggggagaaaattttggcattatcg
ccgcctggaagattaaacttgtcgatgtaccatccaaatc
tacaatattttcagtgaagaagaacatggaaattcatggt
ttggtaaaattattcaacaagtggcagaacatcgcttata
aatatgataaagatttggttctaatgactcattttataac
aaaaaacattacagacaatcacggtaagaataaaactacc
gttcacggatatttcagttcaatcttccatggaggcgttg
attcattggttgatttaatgaacaaatcgtttcctgagct
tggtatcaaaaaaaccgattgcaaggaatttagctggata
gacacaacgattttctactctggtgtagtgaattttaata
cggcaaatttcaagaaggaaatactattagatagaagtgc
tggcaaaaagactgcattttctattaaattagattacgtg
aagaaacccattcctgaaactgccatggttaagattttag
aaaagttgtacgaggaggacgtaggcgcagggatgtatgt
tctgtatccatatggaggtattatggaagaaataagtgag
tctgctattccattcccacatcgtgcgggtatcatgtatg
aactgtggtatactgcatcctgggaaaagcaagaagataa
tgaaaaacacattaactgggttcgcagcgtgtacaatttt
acgacaccgtacgtcagccaaaaccctagactagcttatt
tgaattacagagatcttgacctgggaaaaaccaaccatgc
gtcaccgaataactacacacaggcgcggatatggggcgaa
aagtattttggtaagaacttcaataggttggttaaagtca
aaactaaagtggaccccaataatttttttcgtaatgaaca
atcgatcccacccttacctccacatcaccat.

In some embodiments, a THCAS comprises each of: SEQ ID NO: 203; the MFalpha2 signal peptide; and the HDEL signal peptide. In some embodiments, such a THCAS comprises the amino acid sequence shown below, in which signal peptides are underlined and bolded:

(SEQ ID NO: 253)
MKFISTFLTFILAAVSVTANPRENFLKCFSKHIPNNVANP
KLVYTQHDQLYMSILNSTIQNLRFISDTTPKPLVIVTPSN
NSHIQATILCSKKVGLQIRTRSGGHDAEGMSYISQVPFVV
VDLRNMHSIKIDVHSQTAWVEAGATLGEVYYWINEKNENL
SFPGGYCPTVGVGGHFSGGGYGALMRNYGLAADNIIDAHL
VNVDGKVLDRKSMGEDLFWAIRGGGGENFGIIAAWKIKLV
DVPSKSTIFSVKKNMEIHGLVKLENKWQNIAYKYDKDLVL
MTHFITKNITDNHGKNKTTVHGYFSSIFHGGVDSLVDLMN
KSFPELGIKKTDCKEFSWIDTTIFYSGVVNFNTANFKKEI
LLDRSAGKKTAFSIKLDYVKKPIPETAMVKILEKLYEEDV
GAGMYVLYPYGGIMEEISESAIPFPHRAGIMYELWYTASW
EKQEDNEKHINWVRSVYNFTTPYVSQNPRLAYLNYRDLDL
GKTNHASPNNYTQARIWGEKYFGKNFNRLVKVKTKVDPNN
FFRNEQSIPPLPPHHHHDEL

A non-limiting example of a nucleic acid sequence encoding SEQ ID NO: 253 is shown below, in which sequences encoding signal peptides are underlined and bolded:

(SEQ ID NO: 246)
atgaagtttatcagtaccttcttgacctt
tatcttggccgctgtctccgtaaccgct
aatcctcgtgagaactttctcaaatgcttctccaagcatatac
ccaataacgttgcaaatccgaaattggtatatacgcagcacgaccaactg
tacatgtcaatcttaaatagtactattcaaaaccttcgatttatttcgga
taccacaccaaagcctttggtgatagttactccatctaacaattctcata
tccaagcgacaattttatgtagcaaaaaggtcggcctacagattagaacc
cgcagtgggggtcacgatgccgaaggtatgtcctatatatctcaagtacc
attcgtcgtggttgacttaaggaacatgcattcaattaaaatcgatgtgc
actcgcaaactgcttgggttgaagctggagcaacgttaggtgaggtatac
tattggattaatgaaaagaatgaaaatttgtcttttccgggaggttactg
tcccaccgttggcgtgggcggtcatttttcagggggagggtatggagccc
tcatgagaaactacggtctagcagctgataatataatagacgctcatctt
gtcaatgttgatggtaaagtcctggacagaaaaagcatgggtgaggatct
attctgggcgatcaggggtggcgggggagaaaattttggcattatcgccg
cctggaagattaaacttgtcgatgtaccatccaaatctacaatattttca
gtgaagaagaacatggaaattcatggtttggtaaaattattcaacaagtg
gcagaacatcgcttataaatatgataaagatttggttctaatgactcatt
ttataacaaaaaacattacagacaatcacggtaagaataaaactaccgtt
cacggatatttcagttcaatcttccatggaggcgttgattcattggttga
tttaatgaacaaatcgtttcctgagcttggtatcaaaaaaaccgattgca
aggaatttagctggatagacacaacgattttctactctggtgtagtgaat
tttaatacggcaaatttcaagaaggaaatactattagatagaagtgctgg
caaaaagactgcattttctattaaattagattacgtgaagaaacccattc
ctgaaactgccatggttaagattttagaaaagttgtacgaggaggacgta
ggcgcagggatgtatgttctgtatccatatggaggtattatggaagaaat
aagtgagtctgctattccattcccacatcgtgcgggtatcatgtatgaac
tgtggtatactgcatcctgggaaaagcaagaagataatgaaaaacacatt
aactgggttcgcagcgtgtacaattttacgacaccgtacgtcagccaaaa
ccctagactagcttatttgaattacagagatcttgacctgggaaaaacca
accatgcgtcaccgaataactacacacaggcgcggatatggggcgaaaag
tattttggtaagaacttcaataggttggttaaagtcaaaactaaagtgga
ccccaataatttttttcgtaatgaacaatcgatcccacccttacctccac
atcaccatcatgatgaatta.

In some embodiments, a THCAS comprises the amino acid sequence of any one of SEQ ID NOs: 14, 37-40, 42, 43, 138, 140, 141, 144, 155, 158, 164, 178, 198, 199, 200, 203, 285-313, 474-487, 490, 491, 499, 501, 502, 504, 505, or 553-605.

In some embodiments, a THCAS comprises the nucleotide sequence of any one of SEQ ID NOs: 27-31, 33, 34, 47, 49, 50, 53, 64, 67, 73, 87, 107, 108, 109, 112, 255-283, 332-345, 348-349, 357, 359, 360, 362, 363, or 411-463.

In some embodiments, a THCAS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 14, 20-24, 26-31, 33-34, 37-40, 42, 43, 47, 49, 50, 53, 64, 67, 73, 87, 107, 108, 109, 112, 138, 140, 141, 144, 155, 158, 164, 178, 198, 200, 203, 226-239, 240-253, 255-283, 285-313, 332-345, 348-349, 357, 359-360, 362-363, 411-463, 474-487, 490, 491, 499, 501, 502, 504, 505, or 553-605, or to any TS disclosed in this application. In some embodiments, a THCAS comprises a sequence that is at most 5%, at most 10%, at most 15%, at most 20%, at most 25%, at most 30%, at most 35%, at most 40%, at most 45%, at most 50%, at most 55%, at most 60%, at most 65%, at most 70%, at most 71%, at most 72%, at most 73%, at most 74%, at most 75%, at most 76%, at most 77%, at most 78%, at most 79%, at most 80%, at most 81%, at most 82%, at most 83%, at most 84%, at most 85%, at most 86%, at most 87%, at most 88%, at most 89%, at most 90%, at most 91%, at most 92%, at most 93%, at most 94%, at most 95%, at most 96%, at most 97%, at most 98%, at most 99%, or is 100% identical, including all values in between, to one or more of SEQ ID NOs: 14, 20-24, 26-31, 33-34, 37-40, 42, 43, 47, 49, 50, 53, 64, 67, 73, 87, 107, 108, 109, 112, 138, 140, 141, 144, 155, 158, 164, 178, 194-222, 226-239, 240-253, 255-283, 285-313, 332-345, 348-349, 357, 359-360, 362-363, 370, 373-375, 379, 380, 382, 384-387, 390, 392-394, 396, 400-403, 406-463, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, 884-913, 954-1058, 1060-1067, 1069-1071, 1076, 1078, 1080, 1082, 1084-1088, 1090, 1093-1094, 1101, 1104, 1106-1107, 1132, or 1140-1169 or to any TS disclosed in this application. In some embodiments, a THCAS comprises a sequence that is 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, including all values in between, to one or more of SEQ ID NOs: 14, 20-24, 26-31, 33-34, 37-40, 42, 43, 47, 49, 50, 53, 64, 67, 73, 87, 107, 108, 109, 112, 138, 140, 141, 144, 155, 158, 164, 178, 194-222, 226-239, 240-253, 255-283, 285-313, 332-345, 348-349, 357, 359-360, 362-363, 370, 373-375, 379, 380, 382, 384-387, 390, 392-394, 396, 400-403, 406-463, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, 884-913, 954-1058, 1060-1067, 1069-1071, 1076, 1078, 1080, 1082, 1084-1088, 1090, 1093-1094, 1101, 1104, 1106-1107, 1132, or 1140-1169 or to any TS disclosed in this application.

In some embodiments, a THCAS sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 226-239, or 240-253 includes a signal peptide that comprises SEQ ID NO: 16 or a sequence that has no more than two amino acid substitutions, insertions, additions, or deletions relative to the sequence of SEQ ID NO: 16. In some embodiments, the signal peptide that comprises SEQ ID NO: 16 or a sequence that has no more than two amino acid substitutions, insertions, additions, or deletions relative to the sequence of SEQ ID NO: 16 is located at the N-terminus of the THCAS sequence. For example, the signal peptide that comprises SEQ ID NO: 16 or a sequence that has no more than two amino acid substitutions, insertions, additions, or deletions relative to the sequence of SEQ ID NO: 16 may start at position 2 of the THCAS sequence following a methionine residue.

In some embodiments, a THCAS sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 226-239, or 240-253 includes a signal peptide that comprises SEQ ID NO: 17 or a sequence that has no more than one amino acid substitution, insertion, addition, or deletion relative to the sequence of SEQ ID NO: 17. In some embodiments, the signal peptide that comprises SEQ ID NO: 17 or a sequence that has no more than one amino acid substitution, insertion, addition, or deletion relative to the sequence of SEQ ID NO: 17 is located at the C-terminus of the sequence that is at least 90% identical to one or more of SEQ ID NOs: 226-239, or 240-253.

In some embodiments, a THCAS comprises a sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more SEQ ID NOs: 14, 37-40, 42, 43, 138, 140, 141, 144, 155, 158, 164, 178, 198-200, 203, 285-313, 474-487, 490, 491, 499, 501, 502, 504, 505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, or 884-913, wherein the sequence is linked to one or more signal peptides. In some embodiments, a signal peptide that comprises SEQ ID NO: 16 or a sequence that has no more than two amino acid substitutions, insertions, additions, or deletions relative to the sequence of SEQ ID NO: 16 is linked to the N-terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 14, 37-40, 42, 43, 138, 140, 141, 144, 155, 158, 164, 178, 198-200, 203, 285-313, 474-487, 490, 491, 499, 501, 502, 504, 505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, or 884-913. In some embodiments, a methionine residue is added to the N-terminus of SEQ ID NO: 16. In some embodiments, a signal peptide that comprises SEQ ID NO: 17 or a sequence that has no more than one amino acid substitution, insertion, addition, or deletion relative to the sequence of SEQ ID NO: 17 is linked to the carboxyl terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 14, 37-40, 42, 43, 138, 140, 141, 144, 155, 158, 164, 178, 198-200, 203, 285-313, 474-487, 490, 491, 499, 501, 502, 504, 505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, or 884-913.

In some embodiments, relative to SEQ ID NO: 14, SEQ ID NO: 284, SEQ ID NO: 20 or SEQ ID NO: 21, a THCAS comprises an amino acid substitution, deletion, or insertion at a residue corresponding to position 1, 2, 3, 4, 5, 6, 8, 10, 12, 13, 14, 15, 16, 17, 18, 19, 20, 21, 22, 23, 24, 26, 27, 28, 29, 30, 31, 33, 34, 35, 37, 39, 41, 48, 49, 51, 55, 58, 60, 61, 62, 70, 72, 74, 75, 76, 81, 88, 89, 91, 94, 97, 100, 101, 102, 104, 105, 106, 108, 110, 111, 112, 113, 114, 115, 116, 117, 119, 122, 123, 125, 127, 130, 132, 133, 135, 137, 138, 139, 140, 141, 142, 145, 147, 149, 150, 164, 165, 168, 169, 172, 173, 175, 176, 177, 180, 181, 183, 184, 185, 187, 193, 201, 208, 209, 212, 214, 215, 217, 222, 225, 226, 227, 229, 231, 233, 235, 236, 238, 239, 241, 242, 243, 244, 245, 246, 247, 250, 251, 253, 254, 255, 256, 257, 260, 261, 262, 263, 264, 265, 266, 267, 268, 269, 270, 271, 272, 273, 274, 275, 277, 278, 279, 281, 282, 283, 284, 286, 287, 288, 290, 292, 293, 294, 295, 297, 298, 299, 301, 302, 309, 310, 311, 312, 315, 317, 322, 323, 324, 326, 327, 328, 329, 330, 331, 332, 333, 334, 335, 336, 337, 338, 339, 340, 341, 344, 346, 347, 348, 349, 350, 351, 352, 353, 354, 355, 357, 361, 362, 365, 366, 368, 369, 370, 371, 372, 373, 374, 376, 377, 379, 380, 381, 382, 383, 384, 385, 386, 387, 389, 394, 396, 401, 402, 411, 412, 414, 415, 416, 418, 419, 420, 422, 423, 424, 425, 426, 427, 428, 429, 430, 431, 432, 433, 434, 436, 437, 439, 440, 441, 447, 448, 451, 452, 459, 461, 463, 464, 465, 467, 468, 469,470, 471, 473, 474, 477, 484,485, 488, 492, 496, 497, 500, 505, 511, 513, 514, 515, 516, and/or 517 in SEQ ID NO: 14, SEQ ID NO: 284, SEQ ID NO: 20 or SEQ ID NO: 21. In some embodiments, a THCAS comprises the amino acid residue that is present in positions 14, 37-43, 141, 144, 155, 158, 198, 200 or 203 of SEQ ID NO: 14 at a position corresponding to position 1, 2, 3, 4, 5, 6, 8, 10, 12, 13, 14, 15, 16, 17, 18, 19, 20, 21, 22, 23, 24, 26, 27, 28, 29, 30, 31, 33, 34, 35, 37, 39, 41, 48, 49, 51, 55, 58, 60, 61, 62, 70, 72, 74, 75, 76, 81, 88, 89, 91, 94, 97, 100, 101, 102, 104, 105, 106, 108, 110, 111, 112, 113, 114, 115, 116, 117, 119, 122, 123, 125, 127, 130, 132, 133, 135, 137, 138, 139, 140, 141, 142, 145, 147, 149, 150, 164, 165, 168, 169, 172, 173, 175, 176, 177, 180, 181, 183, 184, 185, 187, 193, 201, 208, 209, 212, 214, 215, 217, 222, 225, 226, 227, 229, 231, 233, 235, 236, 238, 239, 241, 242, 243, 244, 245, 246, 247, 250, 251, 253, 254, 255, 256, 257, 260, 261, 262, 263, 264, 265, 266, 267, 268, 269, 270, 271, 272, 273, 274, 275, 277, 278, 279, 281, 282, 283, 284, 286, 287, 288, 290, 292, 293, 294, 295, 297, 298, 299, 301, 302, 309, 310, 311, 312, 315, 317, 322, 323, 324, 326, 327, 328, 329, 330, 331, 332, 333, 334, 335, 336, 337, 338, 339, 340, 341, 344, 346, 347, 348, 349, 350, 351, 352, 353, 354, 355, 357, 361, 362, 365, 366, 368, 369, 370, 371, 372, 373, 374, 376, 377, 379, 380, 381, 382, 383, 384, 385, 386, 387, 389, 394, 396, 401, 402, 411, 412, 414, 415, 416,418, 419, 420, 422, 423, 424, 425, 426, 427, 428, 429, 430, 431, 432, 433, 434, 436, 437, 439, 440, 441, 447, 448, 451, 452, 459, 461, 463, 464, 465, 467, 468, 469, 470, 471, 473, 474, 477, 484, 485, 488, 492, 496, 497, 500, 505, 511, 513, 514, 515, 516, and/or 517 in SEQ ID NO: 21.

Additional non-limiting examples of THCAS enzymes may also be found in U.S. Pat. No. 9,512,391 and U.S. Patent Publication No. 2018/0179564, which are incorporated by reference in this application in their entireties.

In some embodiments, a THCAS comprises an amino acid deletion or substitution at a residue corresponding to a position shown in Table 17, Table 18, or Table 19.

In some embodiments, a THCAS comprises an amino acid substitution, addition, deletion or insertion at a residue corresponding to position 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 425, 430, 443, 446, 447, 459, 462, 464, 465, 469, 475, 479, 489, 491, 492, 493, 494, 495, 496, 499, 516, 524, 528, 542, 543, and/or 544 in SEQ ID NO: 14.

In some embodiments, the THCAS comprises the amino acid Q at a residue corresponding to position 31 in SEQ ID NO: 14; the amino acid H or Q at a residue corresponding to position 36 in SEQ ID NO: 14; the amino acid E or Q at a residue corresponding to position 40 in SEQ ID NO: 14; the amino acid Y at a residue corresponding to position 41 in SEQ ID NO: 14, the amino acid T at a residue corresponding to position 44 in SEQ ID NO: 14; the amino acid A or P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14, the amino acid P or S at a residue corresponding to position 58 in SEQ ID NO: 14; the amino acid F at a residue corresponding to position 59 in SEQ ID NO: 14; the amino acid S at a residue corresponding to position 61 in SEQ ID NO: 14; the amino acid L or V at a residue corresponding to position 63 in SEQ ID NO: 14; the amino acid T at a residue corresponding to position 74 in SEQ ID NO: 14; the amino acid N at a residue corresponding to position 76 in SEQ ID NO: 14; the amino acid I at a residue corresponding to position 85 in SEQ ID NO: 14; the amino acid L at a residue corresponding to position 88 in SEQ ID NO: 14; the amino acid D, E, or H at a residue corresponding to position 89 in SEQ ID NO: 14; the amino acid E or V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid A at a residue corresponding to position 100 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid Q at a residue corresponding to position 150 in SEQ ID NO: 14, the amino acid L at a residue corresponding to position 158 in SEQ ID NO: 14; the amino acid A at a residue corresponding to position 173 in SEQ ID NO: 14; the amino acid A at a residue corresponding to position 181 in SEQ ID NO: 14; the amino acid K at a residue corresponding to position 196 in SEQ ID NO: 14; the amino acid D at a residue corresponding to position 211 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid D or P at a residue corresponding to position 250 in SEQ ID NO: 14; the amino acid V at a residue corresponding to position 255 in SEQ ID NO: 14; the amino acid M or R at a residue corresponding to position 257 in SEQ ID NO: 14; the amino acid N at a residue corresponding to position 267 in SEQ ID NO: 14; the amino acid E at a residue corresponding to position 268 in SEQ ID NO: 14; the amino acid V at a residue corresponding to position 273 in SEQ ID NO: 14; the amino acid H at a residue corresponding to position 274 in SEQ ID NO: 14; the amino acid L, M, or T at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid Q at a residue corresponding to position 329 in SEQ ID NO: 14; the amino acid E at a residue corresponding to position 340 in SEQ ID NO: 14; the amino acid Q at a residue corresponding to position 344 in SEQ ID NO: 14; the amino acid L or M at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid K at a residue corresponding to position 382 in SEQ ID NO: 14, the amino acid V at a residue corresponding to position 396 in SEQ ID NO: 14; the amino acid V at a residue corresponding to position 411 in SEQ ID NO: 14; the amino acid V at a residue corresponding to position 417 in SEQ ID NO: 14; the amino acid F at a residue corresponding to position 419 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14, the amino acid I at a residue corresponding to position 489 in SEQ ID NO: 14; the amino acid I or M at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid D, E, F, or P at a residue corresponding to position 494 in SEQ ID NO: 14; the amino acid E or K at a residue corresponding to position 495 in SEQ ID NO: 14; the amino acid N at a residue corresponding to position 496 in SEQ ID NO. 14; the amino acid Q at a residue corresponding to position 499 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid at a residue corresponding to position in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, the THCAS comprises any of the combinations of amino acid substitutions shown in Table 17, Table 18, or Table 19.

In some embodiments, a THCAS comprises relative to SEQ ID NO: 14: R31Q, H56N, I74T, N90V, A250P, S255V, Q475K, T492N, H494E, and A495E; R31Q, M61S, I74T, N90V, A250P, S255V, T492N, and H494E; R31Q, K40Q, H41Y, I74T, N90V, V1291, V288L, K296R, F345L, F360Y, A411V, E424D, H494P, and A495E; R31Q, K40Q, H41Y, 174T, N90V, V129I, V288L, K296R, F345L, F360Y, A411V, E424D, H494P, and A495E; R31Q, K40Q, H41Y, I74T, N90V, V1291, V288L, K296R, F345L, F360Y, A411V, E424D, H494P, and A495E; R31Q, K40Q, H41Y, I74T, N90V, V1291, V288L, K296R, F345L, F360Y, A411V, E424D, H494P, A495E, and Y419F; R31Q, K40Q, H41Y, H56N, I74T, N90V, V129I, V288L, K296R, F345L, F360Y, A411V, E424D, Q475K, H494P, and A495E; R31Q, K40Q, H41Y, I74T, N90V, V129I, S255V, V288L, K296R, T340E, F345L, F360Y, A411V, E424D, Q475K, T492N, H494P, and A495E; R31Q, K40Q, H41Y, V46P, V52I, H56N, M61S, I74T, N90V, V1291, S255V, V288L, K296R, T340E, F345L, F360Y, A411V, E424D, Q475K, T492N, H494P, and A495E; or R31Q, K40Q, H41Y, I74T, N90V, V1291, V288L, K296R, F345L, F360Y, A411V, E424D, H494P, and A495E.

In some embodiments, a THCAS comprises relative to SEQ ID NO: 14: R31Q, K40Q, H41Y, N44T, A47T, P49A, L59F, I74T, V85I, S88L, N90V, A95G, P542L, and H543R; R31Q, K40Q, H41Y, I74T, N90V, V129I, V288L, K296R, F345L, F360Y, A411V, E424D, H494P, and A495E; R31Q, K40Q, H41Y, N44T, A47T, P49A, L59F, I74T, V851, S88L, N90V, A95G, P542L, and H543R; R31Q, K40Q, H41 Y, I74T, N90V, V1291, V288L, K296R, F345L, F360Y, A411V, E424D, H494P, and A495E; R31Q, K40Q, H41Y, I74T, N90V, V1291, V288L, K296R, F345L, F360Y, A411V, E424D, H494P, and A495E; R31Q, K40Q, H41Y, 174T, N90V, V1291, V288L, K296R, F345L, F360Y, A411V, E424D, H494P, and A495E; R31Q, K40Q, H41Y, I74T, N90V, V129I, V288L, K296R, F345L, F360Y, A411V, E424D, H494P, and A495E; R31Q, I74T, N90V, A250D, S255V, T492N, and H494E; I74T, N90V, A250P, and H494E; M61S, N90V, A250D, S255V, Q475K, T492N, and A495E; R31Q, K40Q, H41Y, V46P, H56N, Q58S, M61S, I74T, N90V, V129I, H143E, S255V, V288L, K296R, T340E, F345L, Y354F, F360Y, A411V, E424D, Q475K, T492N, H494P, and A495E; R31Q174T, N90V, A250D, and S255V; R3 1Q, H56N, I74T, N90V, A250P, S255V, Q475K, T492N, H494E, and A495E; R31Q, K40Q, H41Y, I74T, N90V, V129I, V288L, K296R, F345L, F360Y, A411V, E424D, H494P, and A495E; or R31 Q, K40Q, H41Y, H56N, I74T, N90V, V129I, V288L, K296R, F345L, F360Y, A411V, E424D, Q475K, H494P, and A495E; A250D, S255V, and H494E; H56N, I74T, N90V, A250D, S255V, T492N, and H494E; H56N, M61S, I74T, N90V, A250P, S255V, T492N, and H494E; I74T, N90V, A250D, S255V, E424D, T492N, and A495E; I74T, N90V, A250D, S255V, and H494E; I74T, N90V, A250P, S255V, E424D, T492N, H494E, and A495E; M61S, I74T, N90V, A250D, S255V, Q475K, T492N, H494E, and A495E; M61S, I74T, N90V, A250P, S255V, E424D, T492N, and H494E; M61S, I74T, N90V, A250P, S255V, Q475K, T492N, and H494E; M61S, I74T, N90V, H143E, A250P, S255V, Q475K, T492N, H494E, and A495E; N90V, A250D, S255V, Q475K, T492N, H494E, and A495E; N90V, A250P, S255V, Q475K, T492N, and H494E; R31Q, H56N, M61S, I74T, N90V, A250P, S255V, T492N, H494E, and A495E; R31Q, K40Q, H41Y, I74T, D76N, N90V, V129I, V158L, V288L, K296R, T340E, F345L, Y354F, F360Y, A411V, E424D, H494P, and A495E; R31Q, K40Q, H41Y, 174T, N89D, N90V, V129I, H143E, V288L, K296R, F345L, F360Y, A411V, E424D, H494P, and A495E; R31Q, K40Q, H41Y, I74T, N90V, S100A, V1291, H143E, V288L, K296R, F345L, T351I, F360Y, A411V, E424D, T492N, H494P, and A495E; R31Q, K40Q, H41Y, I74T, N90V, V129I, H143E, S255V, V288L, K296R, F345L, F360Y, A411V, E424D, H494P, and A495E; R31Q, K40Q, H41Y, I74T, N90V, V129L, S255V, V288L, K296R, F345L, F360Y, A411V, E424D, T4461, H494P, and A495E; R31Q, K40Q, H41Y, 174T, N90V, V1291, S255V, V288L, K296R, F345L, T3511, F360Y, A411V, E424D, H494P, and A495E; R31Q, K40Q, H41Y, I74T, N90V, V1291, S255V, V288L, K296R, T340E, F345L, F360Y, A411V, E424D, Q475K, T492N, H494P, and A495E; R31Q, K40Q, H41Y, I74T, N90V, V1291, V288L, K296R, F345L, F360Y, A411V, E424D, H494P, and A495E; R31Q, K40Q, H41Y, I74T, N90V, V129I, V288L, K296R, F345L, F360Y, A411V, E424D, H494P, A495E, and H267N; R31Q, K40Q, H41Y, 174T, N90V, V129L, V288L, K296R, F345L, F360Y, A411V, E424D, H494P, A495E, and K491M; R31Q, K40Q, H41Y, I74T, N90V, V1291, V288L, K296R, F345L, F360Y, A411V, E424D, H494P, A495E, and S255V; R31Q, K40Q, H41Y, I74T, N90V, V129I, V288L, K296R, F345L, F360Y, A411V, E424D, H494P, A495E, and Y417V; R31Q, K40Q, H41Y, I74T, N90V, V1291, V288L, K296R, F345L, F360Y, A411V, E424D, H494P, A495E, and Y419F; R31Q, K40Q, H41Y, M61S, I74T, N90V, V1291, S255V, V288L, K296R, F345L, F360Y, A411V, E424D, H494P, and A495E; R31Q, K40Q, H41Y, N44T, A47T, P49A, L59F, I74T, V85I, S88L, N90V, A95G, P542L, and H543R; R31Q, K40Q, H41Y, N44T, A47T, P49A, L59F, I74T, V85I, S88L, N90V, A95G, S255V, T340E, Q475K, T492N, H494E, A495E, P542L, and H543R; R31Q, K40Q, H41Y, N44T, A47T, P49A, L59F, M61S, I74T, V85I, S88L, N90V, A95G, H143E, S255V, E424D, T492N, H494E, P542L, and H543R; R31Q, K40Q, H41Y, N44T, A47T, P49A, L59F, M61S, I74T, V85I, S88L, N90V, A95G, H143E, S255V, T340E, E424D, T492N, H494E, A495E, P542L, and H543R, R31Q, K40Q, H41Y, N44T, A47T, P49A, L59F, M61S, I74T, V85I, S88L, N90V, A95G, S255V, T340E, T492N, H494E, A495E, P542L, and H543R; R31Q, K40Q, H41Y, N44T, V46P, A47T, P49A, H56N, Q58S, L59F, M61S, I74T, V85I, S88L, N90V, A95G, H143E, S255V, Q475K, T492N, H494E, A495E, P542L, and H543R; R31Q, K40Q, H41Y, Q58S, I74T, N90V, V129I, H143E, S255V, V288L, K296R, F345L, F360Y, A411V, E424D, T492N, H494P, and A495E; R31Q, K40Q, H41Y, Q58S, I74T, N90V, V129I, S255V, V288L, K296R, F345L, F360Y, A411V, E424D, Q475K, T492N, H494P, and A495E; R31Q, K40Q, H41Y, Q58S, M61S, I74T, N90V, V129L, H143E, S255V, V288L, K296R, T340E, F345L, F360Y, A411V, E424D, T492N, H494P, and A495E; R31Q, K40Q, H41Y, V46P, H56N, Q58S, M61S, I74T, N90V, V1291, S255V, V288L, K296R, F345L, F360Y, A411V, E424D, T492N, H494P, and A495E; R31Q, K40Q, H41Y, V46P, I74T, N90V, V1291, V288L, K296R, T340E, F345L, F360Y, A411V, E424D, H494P, and A495E; R31Q, K40Q, H41Y, V46P, Q58S, I74T, N90V, V129I, S255V, V288L, K296R, F345L, F360Y, A411V, E424D, T492N, H494P, and A495E; R31Q, K40Q, H41Y, V46P, Q58S, I74T, N90V, V129I, S255V, V288L, K296R, T340E, F345L, F360Y, A411V, E424D, Q475K, T492N, H494P, and A495E; R31Q, K40Q, H41Y, V46P, V52I, H56N, M61S, I74T, N90V, V1291, S255V, V288L, K296R, T340E, F345L, F360Y, A411V, E424D, Q475K, T492N, H494P, and A495E; R31Q, K40Q, H41Y, V46P, V52I, H56N, Q58S, M61S, 174T, N90V, V1291, H143E, S255V, V288L, K296R, F345L, T3511, F360Y, A411V, E424D, Q475K, T492N, H494P, and A495E; R31Q, K40Q, H41Y, V46P, V52I, H56N, Q58S, M61S, 174T, N90V, V1291, S255V, V288L, K296R, F345L, F360Y, A411V, E424D, Q475K, T492N, H494P, and A495E; R31Q, K40Q, H41Y, V52I, M61S, I74T, N90V, V129I, S255V, V288L, K296R, T340E, F345L, F360Y, A411V, E424D, Q475K, T492N, H494P, and A495E; R31Q, M61S, I74T, N90V, A250P, S255V, E424D, Q475K, T492N, and H494E; R31Q, M61S, I74T, N90V, A250P, S255V, Q475K, T492N, H494E, and A495E; R31Q, M61S, I74T, N90V, A250P, S255V, T492N, and H494E; R31Q, V46P, I74T, N90V, A250D, S255V, E424D, Q475K, T492N, H494E, and A495E; R31Q, V46P, I74T, N90V, A250D, S255V, Q475K, T492N, and H494E; R31Q, V46P, M61S, I74T, N90V, A250P, S255V, T492N, H494E, and A495E; V46P, H56N, I74T, N90V, A250P, S255V, Q475K, T492N, H494E, and A495E; V46P, 174T, N90V, A250D, and S255V; V46P, M61 S, I74T, N90V, A250D, S255V, E424D, Q475K, T492N, H494E, and A495E; or V46P, M61S, I74T, N90V, A250P, S255V, T492N, and H494E.

In some embodiments, a THCAS comprises relative to SEQ ID NO: 14: R31Q, A47T, H56N, Q58S, M61S, I74T, N90V, H143E, A250P, S255V, V288L, T340E, F345L, E424D, Q475K, and T492N; R31Q, H56N, Q58S, M61S, 174T, N90V, H143E, A250P, S255V, V288L, T340E, F345L, E424D, Q475K, and T492N; R31Q, A47T, V52I, H56N, Q58S, M61S, I74T, N90V, H143E, A250P, S255V, V288L, T340E, F345L, Q475K, and T492N; A47T, H56N, Q58S, M61S, I74T, N90V, A250D, S255V, F345L, Q475K, and T492N; R31Q, A47T, H56N, Q58S, M61S, I74T, N90V, H143E, A250P, S255V, V288L, T340E, F345L, Q475K, and T492N; R31Q, A47T, H56N, Q58S, M61S, 174T, N90V, A250P, S255V, T340E, F345L, E424D, Q475K, and T492N; R31Q, A47T, H56N, Q58S, M61S, I74T, N90V, H143E, A250P, S255V, T340E, F345L, Q475K, and T492N; R31Q, V52I, H56N, Q58S, M61S, I74T, N90V, A250P, S255V, V288L, F345L, Q475K, and T492N; A47T, H56N, Q58S, M61S, 74T, N90V, A250D, S255V, F345L, E424D, Q475K, and T492N; R31Q, H56N, Q58S, M61S, I74T, N90V, A250P, S255V, V288L, F345L, E4241D, Q475K, and T492N; H56N, Q58S, M61 S, 174T, N90V, H143E, A250D, S255V, V288L, F345L, Q475K, T492N, and A495E; H56N, Q58S, M61S, I74T, N90V, H143E, A2501D, S255V, V288L, F345L, Q475K, and T492N; R31Q, A47T, H56N, Q58S, M61 S, 174T, N90V, A250P, S255V, V288L, T340E, F345L, Q475K, and T492N; R31Q, H56N, Q58S, M61S, 174T, N90V, H143E, A250P, S255V, V288L, Q475K, and T492N; A47T, H56N, Q58S, M61IS, I74T, N90V, A2501, S255V, V288L, F345L, Q475K, and T492N; or R31Q, V52L H56N, M61S, 174T, N90V, A250P, S255V, F345L, Q475K, and T492N.

TABLE 3
Mutations in C. sativa THCAS that demonstrated
increased THCA titer either alone or in combination

| Residue in SEQ | | | | |
|---|---|---|---|---|
| ID NO: 14 | | | | |
| (UniProt | | | | |
| Q8GTB6) | Amino Acid Substitutions | | | |
| R31 | Q | | | |
| K36 | H | Q | | |
| K40 | Q | E | | |
| H41 | Y | | | |
| N44 | I | | | |
| V46 | P | A | | |
| A47 | T | | | |
| P49 | A | | | |
| L51 | F | | | |
| V52 | I | | | |
| H56 | N | | | |
| Q58 | S | P | | |
| L59 | F | | | |
| M61 | S | | | |
| I63 | L | V | | |
| I74 | T | | | |
| D76 | N | | | |
| V85 | I | | | |
| S88 | L | | | |
| N89 | D | E | H | |
| N90 | V | E | | |
| A95 | G | | | |
| T96 | S | | | |
| S100 | A | | | |
| V103 | I | | | |
| A116 | S | | | |
| V129 | I | | | |
| H136 | R | | | |
| H143 | E | | | |
| E150 | Q | | | |
| V158 | L | | | |
| G173 | A | | | |
| V181 | A | | | |
| N196 | K | | | |
| N211 | D | | | |
| N237 | S | | | |
| A242 | V | | | |
| K247 | R | | | |
| A250 | D | P | | |
| S255 | V | | | |
| I257 | M | R | | |
| H267 | N | | | |
| G268 | E | | | |
| F273 | V | | | |
| N274 | H | | | |
| V288 | L | M | T | |
| M290 | F | | | |
| K296 | R | | | |
| H302 | Q | | | |
| V309 | I | | | |
| G311 | S | | | |
| H318 | L | | | |
| N329 | Q | | | |
| T340 | E | | | |
| E344 | Q | | | |
| F345 | L | M | | |
| T351 | I | | | |
| Y354 | F | | | |
| F360 | Y | | | |
| N361 | D | | | |
| A363 | T | | | |
| K377 | Q | | | |
| K378 | N | | | |
| T379 | A | | | |
| S382 | K | | | |
| A396 | V | | | |
| A411 | V | | | |
| Y417 | V | | | |
| Y419 | F | | | |
| E424 | D | | | |
| L443 | I | | | |
| T446 | I | | | |
| I459 | L | | | |
| V462 | I | | | |
| S464 | N | | | |
| T469 | M | | | |
| L479 | M | | | |
| Q475 | K | | | |
| K491 | M | | | |
| T492 | N | | | |
| H494 | E | P | F | D |
| A495 | E | K | | |
| N499 | Q | | | |
| N528 | D | | | |
| P542 | L | R | | |
| H543 | R | | | |
| H544 | R | | | |

In some embodiments, one or more amino acid substitutions at particular residues relative to SEQ ID NO: 14 may change the polarity of the residue and alter the stability and/or functionality of a THCAS. Without wishing to be bound by theory, mutations that map to the surface of the tertiary structure of THCAS may, alone or in combination, help solubilize or stabilize the enzyme and result in increased THCA and/or THCVA titer. In some embodiments, one or more amino acid substitutions include K40Q, V52L, H56N, A250D, V288L, T340E, F345L, F360Y, Y419F, E424D, Q475K, T492N, and/or H494E relative to SEQ ID NO: 14. In some embodiments, an amino acid substitution at residue K40 relative to SEQ ID NO. 14 affects the polarity of the residue. For example, the amino acid substitution K40Q relative to SEQ ID NO: 14 switches the residue from a positively charged polar residue to an uncharged polar residue. In some embodiments, an amino acid substitution at residue T340 relative to SEQ ID NO: 14 impacts the polarity of the residue. For example, an amino acid substitution T340E relative to SEQ ID NO: 14 switches the residue from an uncharged polar residue to a negatively charged polar residue, which may favorably counteract the charge of the neighboring positive residues on the surface of the protein (K338, K339, and K343).

In some embodiments, one or more amino acid substitutions increases the product specificity of the THCAS, such as the specificity for a compound of Formula (10), THCA, THCVA or a combination thereof, as compared to a THCAS without such a substitution. In some embodiments, one or more amino acid substitutions increases the product specificity of THCVA. In some embodiments, the one or more amino acid substitutions include: N44T, A47T, P49A, Q58S, L59F, V85I, S88L, A95G, H143E, A250D, Y354F, P542L, and/or H543R relative to SEQ ID NO: 14. In some embodiments, the amino acid at residue Y354 relative to SEQ ID NO: 14 may directly interact with THCA or THCVA. An amino acid substitution at residue Y354 relative to SEQ ID NO: 14 may affect the polarity of the residue. In some embodiments, an amino acid substitution at residue Y354F relative to SEQ ID NO: 14 may change the residue from polar to nonpolar, which may alter the hydrophobicity of the binding pocket.

In some embodiments, a THCAS comprises an amino acid substitution, addition, deletion or insertion at a residue corresponding to position 61, 164, 301, 325, and/or 495 in SEQ ID NO: 20.

In some embodiments, the THCAS comprises the amino acid Q at a residue corresponding to position 61 in SEQ ID NO: 20; the amino acid Q at a residue corresponding to position 164 in SEQ ID NO: 20; the amino acid Q at a residue corresponding to position 301 in SEQ ID NO: 20; the amino acid Q at a residue corresponding to position 325 in SEQ ID NO: 20; and/or the amino acid Q at a residue corresponding to position 495 in SEQ ID NO: 20.

### Cannabidiolic Acid Synthase (CBDAS)

A host cell described in this application may comprise a TS that is a cannabidiolic acid synthase (CBDAS). As used in this application, a “CBDAS” refers to an enzyme that is capable of catalyzing oxidative cyclization of a prenyl moiety (e.g., terpene) of a compound of Formula (8) to produce a compound of Formula 9. In some embodiments, a compound of Formula 9 is a compound of Formula (9a) (cannabidiolic acid (CBDA)), CBDVA, or CBDP. A CBDAS may use cannabigerolic acid (CBGA) or cannabinerolic acid as a substrate. In some embodiments, a cannabidiolic acid synthase is capable of oxidative cyclization of cannabigerolic acid (CBGA) to produce cannabidiolic acid (CBDA). In some embodiments, the CBDAS may catalyze the oxidative cyclization of other substrates, such as 3-geranyl-2,4-dihydro-6-alkylbenzoic acids like cannabigerovarinic acid (CBGVA). In some embodiments, the CBDAS exhibits specificity for CBGA substrates.

In some embodiments, a Cannabis CBDAS is encoded by the CBDAS gene and is a flavoenzyme. A non-limiting example of a Cannabis CBDAS is provided by UniProtKB-A6P6V9 (SEQ ID NO: 13) from C. sativa:

MKCSTFSFWFVCKIIFFFFSFNIQTSIANPRENFLKCFS
QYIPNNATNLKLVYTQNNPLYMSVLNSTIHNLRFTSDTTPKPLVIVTPSH
VSHIQGTILCSKKVGLQIRTRSGGHDSEGMSYISQVPFVIVDLRNMRSIK
IDVHSQTAWVEAGATLGEVYYWVNEKNENLSLAAGYCPTVCAGGHFGGGG
YGPLMRNYGLAADNIIDAHLVNVHGKVLDRKSMGEDLFWALRGGGAESFG
IIVAWKIRLVAVPKSTMFSVKKIMEIHELVKLVNKWQNIAYKYDKDLLLM
THFITRNITDNQGKNKTAIHTYFSSVFLGGVDSLVDLMNKSFPELGIKKT
DCRQLSWIDTIIFYSGVVNYDTDNFNKEILLDRSAGQNGAFKIKLDYVKK
PIPESVFVQILEKLYEEDIGAGMYALYPYGGIMDEISESAIPFPHRAGIL
YELWYICSWEKQEDNEKHLNWIRNIYNFMTPYVSKNPRLAYLNYRDLDIG
INDPKNPNNYTQARIWGEKYFGKNFDRLVKVKTLVDPNNFFRNEQSIPPL
PRHRH

In some embodiments, a Cannabis CBDAS comprises the following sequence:

(SEQ ID NO: 136)
NPRENFLKCFSQYIPNNATNLKLVYTQNNPLYMSVLNSTIHNLRFTSDTT
PKPLVIVTPSHVSHIQGTILCSKKVGLQIRTRSGGHDSEGMSYISQVPFV
IVDLRNMRSIKIDVHSQTAWVEAGATLGEVYYWVNEKNENLSLAAGYCPT
VCAGGHFGGGGYGPLMRNYGLAADNIIDAHLVNVHGKVLDRKSMGEDLFW
ALRGGGAESFGIIVAWKIRLVAVPKSTMFSVKKIMEIHELVKLVNKWQNI
AYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNG
AFKIKLDYVKKPIPESVFVQILEKLYEEDIGAGMYALYPYGGIMDEISES
AIPFPHRAGILYELWYICSWEKQEDNEKHLNWIRNIYNFMTPYVSKNPRL
AYLNYRDLDIGINDPKNPNNYTQARIWGEKYFGKNFDRLVKVKTLVDPNN
FFRNEQSIPPLPRHRH

As described in the Examples section, novel CBDAS enzymes were identified in this disclosure that are capable of catalyzing the conversion of a compound of Formula (8) to produce a compound of Formula (9) and that can be functionally expressed in host cells. Without being bound by a particular theory, the novel CBDAS enzymes disclosed in this application may be useful for engineering to alter the activity and/or abundance of the CBDAS (e.g., change the product profile, substrate profile, and/or kinetics (e.g., Kcat/Vmax and/or Kd) of the TS).

In some embodiments, a CBDAS comprises the amino acid sequence of any one of SEQ ID NOs: 36, 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and/or 948.

In some embodiments, a CBDAS comprises the nucleotide sequence of any one of SEQ ID NOs: 27, 52, 58, 60-62, 65, 69, 72, 74, 75, 77, 79-81, 84-89, 91-106, 110-111, 113-114, 116-134, 322-331, 336-338, 342-343, 345-347, 350-356, 358, 361, 364-406, 408-410, 414, 416, 423, 425, 427-428, 430-436, 440, 442, 444, 446, 449, 451453, 455, 458, 460, 462, 463, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and/or 1204.

In some embodiments, a CBDAS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 13, 27, 36, 52, 58, 60-62, 65, 69, 72, 74, 75, 77, 79-81, 84-89, 91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408410, 414, 416, 423, 425, 427428, 430436, 440, 442, 444, 446, 449, 451453, 455, 458, 460, 462, 463, 464-473, 478-480, 484485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and/or 1204 or to any TS disclosed in this application. In some embodiments, a CBDAS comprises a sequence that is at most 5%, at most 10%, at most 15%, at most 20%, at most 25%, at most 30%, at most 35%, at most 40%, at most 45%, at most 50%, at most 55%, at most 60%, at most 65%, at most 70%, at most 71%, at most 72%, at most 73%, at most 74%, at most 75%, at most 76%, at most 77%, at most 78%, at most 79%, at most 80%, at most 81%, at most 82%, at most 83%, at most 84%, at most 85%, at most 86%, at most 87%, at most 88%, at most 89%, at most 90%, at most 91%, at most 92%, at most 93%, at most 94%, at most 95%, at most 96%, at most 97%, at most 98%, at most 99%, or is 100% identical, including all values in between, to one or more of SEQ ID NOs: 13, 27, 36, 52, 58, 60-62, 65, 69, 72, 74, 75, 77, 79-81, 84-89, 91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408-410, 414, 416, 423, 425, 427-428, 430-436, 440, 442, 444, 446, 449, 451-453, 455, 458, 460, 462, 463, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and/or 1204 or to any TS disclosed in this application. In some embodiments, a CBDAS comprises a sequence that is 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, including all values in between, to one or more of SEQ ID NOs: 13, 27, 36, 52, 58, 60-62, 65, 69, 72, 74, 75, 77, 79-81, 84-89, 91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408-410, 414, 416, 423, 425, 427-428, 430-436, 440, 442, 444, 446, 449, 451-453, 455, 458, 460, 462, 463, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124,1126-1135, 1137, 1139, 1169-1188,1195-1197, 1199-1201, 1202, and/or 1204 or to any TS disclosed in this application.

In some embodiments, a CBDAS comprises a sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more SEQ ID NOs: 13, 27, 36, 52, 58, 60-62, 65, 69, 72, 74, 75, 77, 79-81, 84-89, 91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408-410, 414, 416, 423, 425, 427-428, 430-436, 440, 442, 444, 446, 449, 451-453, 455, 458, 460, 462, 463, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941,944,945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and/or 1204 wherein the sequence is linked to one or more signal peptides. In some embodiments, a signal peptide that comprises SEQ ID NO: 16 or a sequence that has no more than two amino acid substitutions, insertions, additions, or deletions relative to the sequence of SEQ ID NO: 16 is linked to the N-terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 13, 27, 36, 52, 58, 60-62, 65, 69, 72, 74, 75, 77, 79-81, 84-89, 91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408-410, 414, 416, 423, 425, 427-428, 430-436, 440, 442, 444, 446, 449, 451-453, 455, 458, 460, 462, 463, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and/or 1204. In some embodiments, a methionine residue is added to the N-terminus of SEQ ID NO: 16. In some embodiments, a signal peptide that comprises SEQ ID NO: 17 or a sequence that has no more than one amino acid substitution, insertion, addition, or deletion relative to the sequence of SEQ ID NO: 17 is linked to the carboxyl terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 13, 27, 36, 52, 58, 60-62, 65, 69, 72,74,75,77,79-81, 84-89,91-106, 110-111, 113-114, 116-134, 143, 149, 151-153, 156, 160, 163, 165, 166, 168, 170-172, 175-180, 182-197, 201, 204, 205, 207-225, 322-331, 336-338, 342-343, 345-347, 350-356, 358, 361, 364-406, 408-410, 414, 416, 423, 425, 427-428, 430-436, 440, 442, 444, 446, 449, 451-453, 455, 458, 460, 462, 463, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, 948, 974, 1011, 1040, 1042, 1046-1048, 1050, 1051, 1054, 1056, 1057, 1059, 1060, 1062-1066, 1068-1077, 1079, 1081, 1083-1092, 1094, 1095, 1097-1124, 1126-1135, 1137, 1139, 1169-1188, 1195-1197, 1199-1201, 1202, and 1204.

Additional non-limiting examples of CBDAS enzymes may also be found in U.S. Pat. No. 9,512,391 and U.S. Publication No. 2018/0179564, which are incorporated by reference in this application in their entireties.

In some embodiments, a CBDAS comprises an amino acid deletion or substitution at a position shown in Table 17, Table 18, or Table 19.

In some embodiments, a CBDAS comprises an amino acid substitution, addition, deletion or insertion at a residue corresponding to position 31, 47, 49, 50, 56, 57, 58, 69, 79, 89, 90, 95, 100, 103, 106, 116, 124, 143, 150, 162, 166, 167, 168, 171, 172, 175, 180, 184, 196, 211, 213, 216, 230, 250, 253, 263, 273, 283, 287, 290, 292, 319, 322, 339, 343, 344, 352, 353, 376, 377, 378, 380, 386, 394, 397, 407, 409, 410,411, 414, 415, 416, 418,442, 441, 445, 446, 450, 452, 454, 467, 479, 481, 486, 490, 492, 504, 512 527 and/or 542 in SEQ ID NO: 13.

In some embodiments, the CBDAS comprises the amino acid Q at a residue corresponding to position 31 in SEQ ID NO: 13; the amino acid A at a residue corresponding to position 47 in SEQ ID NO: 13; the amino acid P at a residue corresponding to position 49 in SEQ ID NO: 13; the amino acid N at a residue corresponding to position 50 in SEQ ID NO: 13; the amino acid H at a residue corresponding to position 56 in SEQ ID NO: 13; the amino acid D at a residue corresponding to position 57 in SEQ ID NO: 13; the amino acid Q at a residue corresponding to position 58 in SEQ ID NO: 13; the amino acid R or Q at a residue corresponding to position 69 in SEQ ID NO: 13; the amino acid G at a residue corresponding to position 79 in SEQ ID NO: 13; the amino acid N, D, E, Q, or R at a residue corresponding to position 89 in SEQ ID NO: 13; the amino acid C at a residue corresponding to position 90 in SEQ ID NO: 13; the amino acid A at a residue corresponding to position 95 in SEQ ID NO: 13; the amino acid A at a residue corresponding to position 100 in SEQ ID NO: 13; the amino acid H at a residue corresponding to position 103 in SEQ ID NO: 13; the amino acid E at a residue corresponding to position 106 in SEQ ID NO: 13; the amino acid A or G at a residue corresponding to position 116 in SEQ ID NO: 13, the amino acid N or M at a residue corresponding to position 124 in SEQ ID NO: 13; the amino acid E at a residue corresponding to position 143 in SEQ ID NO: 13; the amino acid Q at a residue corresponding to position 150 in SEQ ID NO: 13; the amino acid S at a residue corresponding to position 166 in SEQ ID NO: 13; the amino acid K at a residue corresponding to position 167 in SEQ ID NO: 13; the amino acid T at a residue corresponding to position 168 in SEQ ID NO: 13; the amino acid F at a residue corresponding to position 171 in SEQ ID NO: 13; the amino acid P at a residue corresponding to position 172 in SEQ ID NO: 13; the amino acid F at a residue corresponding to position 175 in SEQ ID NO: 13; the amino acid G at a residue corresponding to position 180 in SEQ ID NO: 13; the amino acid F at a residue corresponding to position 184 in SEQ ID NO: 13; the amino acid K at a residue corresponding to position 196 in SEQ ID NO: 13; the amino acid D at a residue corresponding to position 211 in SEQ ID NO: 13; the amino acid N at a residue corresponding to position 213 in SEQ ID NO: 13; the amino acid L at a residue corresponding to position 216 in SEQ ID NO: 13; the amino acid I at a residue corresponding to position 230 in SEQ ID NO: 13; the amino acid R at a residue corresponding to position 250 in SEQ ID NO: 13; an insertion of an amino acid S at a residue corresponding to position 253 in SEQ ID NO: 13; the amino acid L at a residue corresponding to position 263 in SEQ ID NO: 13; the amino acid H at a residue corresponding to position 273 in SEQ ID NO: 13; the amino acid P at a residue corresponding to position 283 in SEQ ID NO: 13; the amino acid T at a residue corresponding to position 287 in SEQ ID NO: 13; the amino acid M or A at a residue corresponding to position 290 in SEQ ID NO: 13; the amino acid M at a residue corresponding to position 292 in SEQ ID NO: 13; the amino acid D or N at a residue corresponding to position 319 in SEQ ID NO: 13, the amino acid E at a residue corresponding to position 322 in SEQ ID NO: 13; the amino acid E at a residue corresponding to position 339 in SEQ ID NO: 13; the amino acid E at a residue corresponding to position 343 in SEQ ID NO: 13; the amino acid M at a residue corresponding to position 344 in SEQ ID NO: 13; the amino acid M at a residue corresponding to position 353 in SEQ ID NO: 13; the amino acid L or Y at a residue corresponding to position in SEQ ID NO: 13; the amino acid L, Y, A, G, N, P, R, S, T, or V at a residue corresponding to position 376 in SEQ ID NO: 13; the amino acid F, P, or R at a residue corresponding to position 377 in SEQ ID NO: 13, the amino acid K, R, S, or T at a residue corresponding to position 378 in SEQ ID NO: 13; the amino acid Y at a residue corresponding to position 380 in SEQ ID NO: 13; the amino acid F at a residue corresponding to position 386 in SEQ ID NO: 13; the amino acid E at a residue corresponding to position 394 in SEQ ID NO: 13; the amino acid E or K at a residue corresponding to position 397 in SEQ ID NO: 13; the amino acid E at a residue corresponding to position 407 in SEQ ID NO: 13; the amino acid G at a residue corresponding to position 409 in SEQ ID NO: 13; the amino acid T or V at a residue corresponding to position 410 in SEQ ID NO: 13; the amino acid G at a residue corresponding to position 411 in SEQ ID NO: 13; the amino acid I, L, M, T, or V at a residue corresponding to position 414 in SEQ ID NO: 13; the amino acid M at a residue corresponding to position 415 in SEQ ID NO: 13; the amino acid F, I, or M at a residue corresponding to position 416 in SEQ ID NO: 13; the amino acid F at a residue corresponding to position 418 in SEQ ID NO: 13; the amino acid S or T at a residue corresponding to position 441 in SEQ ID NO: 13, the amino acid I at a residue corresponding to position 442 in SEQ ID NO: 13; the amino acid V at a residue corresponding to position 445 in SEQ ID NO: 13; the amino acid T or V at a residue corresponding to position 446 in SEQ ID NO: 13; the amino acid S at a residue corresponding to position 450 in SEQ ID NO: 13; the amino acid T at a residue corresponding to position 452 in SEQ ID NO: 13; the amino acid A at a residue corresponding to position 454 in SEQ ID NO: 13; the amino acid Y at a residue corresponding to position 467 in SEQ ID NO: 13; the amino acid S or T at a residue corresponding to position 479 in SEQ ID NO: 13; the amino acid I, M, V, or Y at a residue corresponding to position 481 in SEQ ID NO: 13; the amino acid V at a residue corresponding to position 486 in SEQ ID NO: 13; the amino acid T at a residue corresponding to position 490 in SEQ ID NO: 13, the amino acid N at a residue corresponding to position 492 in SEQ ID NO: 13; the amino acid Q at a residue corresponding to position 504 in SEQ ID NO: 13; the amino acid N at a residue corresponding to position 512 in SEQ ID NO: 13; the amino acid D at a residue corresponding to position 527 in SEQ ID NO: 13; and/or the amino acid M at a residue corresponding to position 542 in SEQ ID NO: 13.

In some embodiments, a CBDAS comprises an amino acid deletion or substitution at a residue corresponding to position 50, 116 or 414 in SEQ ID NO: 13. In some embodiments, the amino acid deletion or substitution comprises K50N, S116A and/or A414V.

In some embodiments, the CBDAS comprises any combination of amino acid substitutions relative to SEQ ID NO: 13 shown in Table 17, Table 18, or Table 19.

In some embodiments, a CBDAS comprises relative to SEQ ID NO: 13: S100A, S116A, and H213N, H69Q, G95A, S116A, T339E, and Q343E; H69Q, G95A, S116A, and T339E; T47A, L49P, N56H, N57D, P58Q, H69Q, H89N, and G95A; G95A, S116A, and Q343E; G95A, S116A, and T339E; K50N, H69Q, G95A, H213N, T339E, and L344M; H69Q, G95A, S116A, H213N, T339E, and Q343E; K50N, H69Q, G95A, and Q343E; K50N, H69Q, G95A, H213N, Q343E, and L344M; G95A, S116A, H213N, T339E, Q343E, and L344M; G95A, S116A, T339E, and Q343E; G95A, S116A, H213N, Q343E, and L344M; G95A, S116A, H213N, T339E, and Q343E; R31Q, T47A, L49P, N56H, N57D, P58Q, H69Q, H89N, and G95A; K50N, G95A, S116A, H213N, L344M, and N527D; G95A, S116A, T339E, Q343E, and L344M; K50N, H69Q, H213N, T339E, Q343E, and A414V; G95A, A414V, and N527D; H69Q, G95A, H213N, S322E, Q343E, L344M, and A414V; G95A, Q343E, G378T, and A414V; K50N, H69Q, G95A, T339E, Q343E, L344M, and A414V; H69Q, G95A, H213N, L344M, and A414V; K50N, H69Q, G95A, Q343E, and A414V; H69Q, G95A, H213N, T339E, Q343E, L344M, and A414V; G95A, H213N, Q343E, and A414V; G95A, H213N, T339E, Q343E, A414V, and D492N; H69Q, G95A, H213N, Q343E, A414V, and N527D; G95A, H213N, T339E, G378T, A410V, A414V, and 1445V; H69Q, G95A, Q343E, A414V, and N527D; K50N, H69Q, G95A, H213N, Q343E, L344M, and A414V; K50N, G95A, and A414V; K50N, H69Q, G95A, H213N, T339E, Q343E, and A414V; G95A, T339E, L344M, and A414V; K50N, H213N, Q343E, L344M, and A414V; G95A, H213N, T339E, Q343E, and A414V; K50N, G95A, H213N, T339E, Q343E, A414V, and D492N; K50N, G95A, H213N, T339E, Q343E, L344M, and A414V; G95A, T339E, Q343E, L344M, and A414V; G95A, Q343E, L344M, and A414V; G95A, H213N, Q343E, L344M, and A414V; G95A, T339E, Q343E, and A414V; G95A, H213N, T339E, L344M, and A414V; K50N, G95A, H213N, Q343E, L344M, and A414V; K50N, G95A, Q343E, L344M, and A414V; or G95A, H213N, T339E, Q343E, L344M, and A414V.

In some embodiments, a CBDAS comprises relative to SEQ ID NO: 13: S100A, S116A, and H213N; H69Q, G95A, S116A, T339E, and Q343E; H69Q, G95A, S116A, and T339E; T47A, L49P, N56H, N57D, P58Q, H69Q, H89N, and G95A; G95A, Si 16A, and Q343E; G95A, S116A, and T339E; K50N, H69Q, G95A, H213N, T339E, and L344M; H69Q, G95A, S116A, H213N, T339E, and Q343E; K50N, H69Q, G95A, and Q343E; K50N, H69Q, G95A, H213N, Q343E, and L344M; G95A, S116A, H213N, T339E, Q343E, and L344M; G95A, S116A, T339E, and Q343E; G95A, S116A, H213N, Q343E, and L344M; G95A, S116A, H213N, T339E, and Q343E; R31Q, T47A, L49P, N56H, N57D, P58Q, H69Q, H89N, and G95A; K50N, G95A, S116A, H213N, L344M, and N527D; G95A, S116A, T339E, Q343E, and L344M; K50N, H69Q, H213N, T339E, Q343E, and A414V; G95A, A414V, and N527D; H69Q, G95A, H213N, S322E, Q343E, L344M, and A414V; G95A, Q343E, G378T, and A414V; K50N, H69Q, G95A, T339E, Q343E, L344M, and A414V; H69Q, G95A, H213N, L344M, and A414V; K50N, H69Q, G95A, Q343E, and A414V; H69Q, G95A, H213N, T339E, Q343E, L344M, and A414V; G95A, H213N, Q343E, and A414V; G95A, H213N, T339E, Q343E, A414V, and D492N; H69Q, G95A, H213N, Q343E, A414V, and N527D; G95A, H213N, T339E, G378T, A410V, A414V, and 1445V; H69Q, G95A, Q343E, A414V, and N527D; K50N, H69Q, G95A, H213N, Q343E, L344M, and A414V; K50N, G95A, and A414V SEQ ID NO: 13, K50N, H69Q, G95A, H213N, T339E, Q343E, and A414V; G95A, T339E, L344M, and A414V; K50N, H213N, Q343E, L344M, and A414V; G95A, H213N, T339E, Q343E, and A414V; K50N, G95A, H213N, T339E, Q343E, A414V, and D492N; K50N, G95A, H213N, T339E, Q343E, L344M, and A414V; G95A, T339E, Q343E, L344M, and A414V; G95A, Q343E, L344M, and A414V; G95A, H213N, Q343E, L344M, and A414V; G95A, T339E, Q343E, and A414V; G95A, H213N, T339E, L344M, and A414V; K50N, G95A, H213N, Q343E, L344M, and A414V; K50N, G95A, Q343E, L344M, and A414V; or G95A, H213N, T339E, Q343E, L344M, and A414V.

In some embodiments, a CBDAS comprises relative to SEQ ID NO. 13: K50N, G95A, N196K, H213N, T339E, Q343E, L344M, and A414V; G95A, Y175F, T339E, Q343E, and A414V; G95A, Si 16A, T339E, Q343E, A414V, and N527D3; G95A, E150Q, V1621, C180G, N196K, N211D, N273H, T339E, Q343E, and A414V; G95A, T339E, Q343E, Q376V, and A414V; K50N, G95A, S100A, E150Q, V1621, C180G, N196K, N211D, H213N, S322E, T339E, Q343E, L344K. A414V, E452T, and I504Q; G95A, N196K, T339E, Q343E, and A414V, K50N, G95A, V103H, H213N, T339E, Q343E, L344M, and A414V, G95A, T339E, Q343E, Q376R, and A414V; or K50N, H213N, L230L, T339E, Q343E, and L344M.

In some embodiments, a CBDAS comprises an amino acid insertion at a residue corresponding to position 253 in SEQ ID NO: 13. In some embodiments, the amino acid S is inserted at a residue corresponding to position 253 in SEQ ID NO: 13.

TABLE 4
Mutations in C. sativa CBDAS that demonstrated
increased CBDA titer either alone or in combination

Residue in SEQ ID

NO: 13 (UniProt

| A6P6V9) | Amino Acid Substitutions | | | | | | | | | |
|---|---|---|---|---|---|---|---|---|---|---|
| R31 | Q | | | | | | | | | |
| T47 | A | | | | | | | | | |
| L49 | P | | | | | | | | | |
| K50 | N | | | | | | | | | |
| N56 | H | | | | | | | | | |
| N57 | D | | | | | | | | | |
| P58 | Q | | | | | | | | | |
| H69 | Q | R | | | | | | | | |
| P79 | G | | | | | | | | | |
| H89 | N | D | E | Q | R | | | | | |
| V90 | C | | | | | | | | | |
| G95 | A | | | | | | | | | |
| S100 | A | | | | | | | | | |
| V103 | H | | | | | | | | | |
| Q106 | E | | | | | | | | | |
| S116 | A | G | | | | | | | | |
| Q124 | N | M | | | | | | | | |
| H143 | E | | | | | | | | | |
| E150 | Q | | | | | | | | | |
| V162 | I | | | | | | | | | |
| N166 | S | | | | | | | | | |
| E167 | K | | | | | | | | | |
| N168 | T | | | | | | | | | |
| L171 | F | | | | | | | | | |
| A172 | P | | | | | | | | | |
| Y175 | F | | | | | | | | | |
| C180 | G | | | | | | | | | |
| H184 | F | | | | | | | | | |
| N196 | K | | | | | | | | | |
| N211 | D | | | | | | | | | |
| H213 | N | | | | | | | | | |
| V216 | L | | | | | | | | | |
| L230 | I | | | | | | | | | |
| A250 | R | | | | | | | | | |
| M263 | L | | | | | | | | | |
| N273 | H | | | | | | | | | |
| D283 | P | | | | | | | | | |
| L287 | T | | | | | | | | | |
| T290 | A | M | | | | | | | | |
| F292 | M | | | | | | | | | |
| G319 | D | N | | | | | | | | |
| S322 | E | | | | | | | | | |
| T339 | E | | | | | | | | | |
| Q343 | E | | | | | | | | | |
| L344 | M | | | | | | | | | |
| Y353 | M | | | | | | | | | |
| Q376 | L | Y | A | G | N | P | R | S | T | V |
| N377 | F | P | R | | | | | | | |
| G378 | T | K | R | S | | | | | | |
| F380 | Y | | | | | | | | | |
| Y386 | F | | | | | | | | | |
| S394 | E | | | | | | | | | |
| V397 | E | K | | | | | | | | |
| D407 | E | | | | | | | | | |
| A410 | V | T | | | | | | | | |
| A414 | V | I | L | M | T | | | | | |
| L415 | M | | | | | | | | | |
| Y416 | F | I | M | | | | | | | |
| Y418 | F | | | | | | | | | |
| E441 | S | T | | | | | | | | |
| L442 | I | | | | | | | | | |
| I445 | V | A | | | | | | | | |
| C446 | T | V | | | | | | | | |
| A479 | S | T | | | | | | | | |
| K450 | S | | | | | | | | | |
| E452 | T | | | | | | | | | |
| N454 | A | | | | | | | | | |
| F467 | Y | | | | | | | | | |
| L481 | I | M | V | Y | | | | | | |
| L486 | V | | | | | | | | | |
| I490 | T | | | | | | | | | |
| D492 | N | | | | | | | | | |
| I504 | Q | | | | | | | | | |
| K512 | N | | | | | | | | | |
| N527 | D | | | | | | | | | |
| H542 | M | | | | | | | | | |

In some embodiments, one or more amino acid substitutions at particular residues relative to SEQ ID NO: 13 may change the polarity of the residue and alter the stability and/or functionality of a CBDAS. Without wishing to be bound by theory, mutations that map to the surface of the tertiary structure of CBDAS may, alone or in combination, help solubilize or stabilize the enzyme and result in increased CBDA and/or CBDVA titer. In some embodiments, one or more of the following amino acid substitutions relative to SEQ ID NO: 13 may change the polarity of the residue and may impact solubilization and/or stabilization of the enzyme: K50N, H213N, S322E, T339E, L344M, and N527D. In some embodiments, one or more of the following amino acid substitutions relative to SEQ ID NO: 13 may change the polarity of the residue and may impact solubilization and/or stabilization of the enzyme: N211D, H213N, and E452T. In some embodiments, an amino acid substitution at residue N211 relative to SEQ ID NO: 13 affects the polarity of the residue. For example, the amino acid substitution N211 D relative to SEQ ID NO: 13 switches the residue from a non-charged polar residue to a negatively charged residue, which may favorably counteract the charge of the neighboring positive residues on the surface of the protein (R108, H213 and K215). In some embodiments, an amino acid substitution at residue H213 relative to SEQ ID NO: 13 affects the polarity of the residue. For example, the amino acid substitution H213N relative to SEQ ID NO: 13 switches the residue from a positively charged residue to a non-charged polar residue, which may favorably minimize the size of a positively charged surface region on the protein consisting of the neighboring positive residues (K101, K102, and K215). In some embodiments, an amino acid substitution at residue E452 relative to SEQ ID NO: 13 affects the polarity of the residue. For example, the amino acid substitution E452T relative to SEQ ID NO: 13 switches the residue from a negatively charged residue to a non-charged polar residue, which may favorably minimize a negatively charged surface region on the protein consisting of the neighboring negative residues (E449 and D453).

In some embodiments, one or more amino acid substitutions at particular residues relative to SEQ ID NO: 13 may change the polarity of the residue and alter the protein folding and/or protein packing of a CBDAS. Without wishing to be bound by theory, mutations that map to the interior of the enzyme may, alone or in combination, impact protein folding and/or protein packing and result in increased CBDA and/or CBDVA titer. In some embodiments, one or more of the following amino acid substitutions relative to SEQ ID NO: 13 may impact folding or packing of the enzyme: S100A and C180G. In some embodiments, an amino acid substitution at residue S100 relative to SEQ ID NO: 13 affects the polarity of the residue. For example, the amino acid substitution S100A relative to SEQ ID NO: 13 switches the residue from a non-charged polar residue to a nonpolar aliphatic residue, which may increase the hydrophobicity of the internal region and may favorably contribute to protein folding and protein packing. In some embodiments, an amino acid substitution at residue C180 relative to SEQ ID NO: 13 affects the polarity of the residue. For example, the amino acid substitution C180G relative to SEQ ID NO: 13 switches the residue from a non-charged polar residue to a nonpolar aliphatic residue, which may increase the hydrophobicity of the internal region and may favorably contribute to protein folding and protein packing.

In some embodiments, one or more amino acid substitutions in a CBDAS increases product specificity of the CBDAS, such as specificity for a compound of formula (9), CBCA, CBDVA, or a combination thereof, as compared to a CBDAS without such a substitution.

In some embodiments, one or more amino acid substitutions in a CBDAS increases product titer, as compared to a CBDAS without such an amino acid substitution. In some embodiments, the one or more amino acid substitutions is at residue A414 relative to SEQ ID NO: 13. In some embodiments, the amino acid substitution is A414V relative to SEQ ID NO: 13.

### Cannabichromenic Acid Synthase (CBCAS)

A host cell described in this application may comprise a TS that is a cannabichromenic acid synthase (CBCAS). As used in this application, a “CBCAS” refers to an enzyme that is capable of catalyzing oxidative cyclization of a prenyl moiety (e.g., terpene) of a compound of Formula (8) to produce a compound of Formula (11). In some embodiments, a compound of Formula (11) is a compound of Formula (11a) (cannabichromenic acid (CBCA)), CBCVA, or a compound of Formula (8) with R as a C7 alkyl (heptyl) group. A CBCAS may use cannabigerolic acid (CBGA) as a substrate. In some embodiments, a CBCAS produces cannabichromenic acid (CBCA) from cannabigerolic acid (CBGA). In some embodiments, the CBCAS may catalyze the oxidative cyclization of other substrates, such as 3-geranyl-2,4-dihydro-6-alkylbenzoic acids like cannabigerovarinic acid (CBGVA), or a substrate of Formula (8) with R as a C7 alkyl (heptyl) group. In some embodiments, the CBCAS exhibits specificity for CBGA substrates.

In some embodiments, a CBCAS is from Cannabis. In C. sativa, an amino acid sequence encoding CBCAS is provided by, and incorporated by reference from, SEQ ID NO:2 disclosed in U.S. Patent Publication No. 2017/0211049. In other embodiments, a CBCAS may be a THCAS described in and incorporated by reference from U.S. Pat. No. 9,359,625. SEQ ID NO:2 disclosed in U.S. Patent Publication No. 2017/0211049 (corresponding to SEQ ID NO: 15 in this application) has the amino acid sequence:

MNCSTFSFWFVCKIIFFFLSFNIQISIANPQENFLKCFS
EYIPNNPANPKFIYTQHDQLYMSVLNSTIQNLRFTSDTTPKPLVIVTPSN
VSHIQASILCSKKVGLQIRTRSGGHDAEGLSYISQVPFAIVDLRNMHTVK
VDIHSQTAWVEAGATLGEVYYWINEMNENFSFPGGYCPTVGVGGHFSGGG
YGALMRNYGLAADNIIDAHLVNVDGKVLDRKSMGEDLFWAIRGGGGENFG
IIAACKIKLVVVPSKATIFSVKKNMEIHGLVKLENKWQNIAYKYDKDLML
TTHFRTRNITDNHGKNKTTVHGYFSSIFLGGVDSLVDLMNKSFPELGIKK
TDCKELSWIDTTIFYSGVVNYNTANFKKEILLDRSAGKKTAFSIKLDYVK
KLIPETAMVKILEKLYEEEVGVGMYVLYPYGGIMDEISESAIPFPHRAGI
MYELWYTATWEKQEDNEKHINWVRSVYNFTTPYVSQNPRLAYLNYRDLDL
GKTNPESPNNYTQARIWGEKYFGKNFNRLVKVKTKADPNNFFRNEQSIPP
LPPRHH.

In some embodiments, a CBCAS comprises the sequence shown below:

(SEQ ID NO: 25)
NPQENFLKCFSEYIPNNPANPKFIYTQHDQLYMSVLNSTIQNLRFTSDT
TPKPLVIVTPSNVSHIQASILCSKKVGLQIRTRSGGHDAEGLSYISQVPF
AIVDLRNMHTVKVDIHSQTAWVEAGATLGEVYYWINEMNENFSFPGGYCP
TVGVGGHFSGGGYGALMRNYGLAADNIIDAHLVNVDGKVLDRKSMGEDLF
WAIRGGGGENFGIIAACKIKLVVVPSKATIFSVKKNMEIHGLVKLFNKWQ
NIAYKYDKDLMLTTHFRTRNITDNHGKNKTTVHGYFSSIFLGGVDSLVDL
MNKSFPELGIKKTDCKELSWIDTTIFYSGVVNYNTANFKKEILLDRSAGK
KTAFSIKLDYVKKLIPETAMVKILEKLYEEEVGVGMYVLYPYGGIMDEIS
ESAIPFPHRAGIMYELWYTATWEKQEDNEKHINWVRSVYNFTTPYVSQNP
RLAYLNYRDLDLGKTNPESPNNYTQARIWGEKYFGKNFNRLVKVKTKADP
NNFFRNEQSIPPLPPRHH.

Additional CBCASs are disclosed in and incorporated by reference from PCT Application No. PCT/US21/24398.

As described in the Examples section, novel CBCAS enzymes were identified in this disclosure that are capable of catalyzing the conversion of a compound of Formula (8) to produce a compound of Formula (11) and that can be functionally expressed in host cells. Without being bound by a particular theory, the novel CBCAS enzymes disclosed in this application may be useful for engineering to alter the activity and/or abundance of the CBCAS (e.g., change the product profile, substrate profile, and/or kinetics (e.g., Kcat/Vmax and/or Kd) of the TS).

In some embodiments, a CBCAS comprises the amino acid sequence of any one of SEQ ID NOs: 15, 39, 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993.

In some embodiments, a CBCAS comprises the nucleotide sequence of any one of SEQ ID NOs: 30, 46-49, 51, 52, 54-59, 63, 66, 68, 70, 71, 73, 76, 78, 82, 83, 86-91, 102, 104, 105, 108, 113-115, 322-324, 346, 347, 350, 351-356, 358, 360, 361, 364-406, 408, 409, 410, 423, 432, 453, 455, 460, 952-1138, and 1189.

In some embodiments, a CBCAS comprises a nucleic acid or protein sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to one or more of SEQ ID NOs: 15, 30, 39, 46-49, 51, 52, 54-59, 63, 66, 68, 70, 71, 73, 76, 78, 82, 83, 86-91, 102, 104, 105, 108, 113-115, 137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189 or to any TS disclosed in this application. In some embodiments, a TS comprises a sequence that is at most 5%, at most 10%, at most 15%, at most 20%, at most 25%, at most 30%, at most 35%, at most 40%, at most 45%, at most 50%, at most 55%, at most 60%, at most 65%, at most 70%, at most 71%, at most 72%, at most 73%, at most 74%, at most 75%, at most 76%, at most 77%, at most 78%, at most 79%, at most 80%, at most 81%, at most 82%, at most 83%, at most 84%, at most 85%, at most 86%, at most 87%, at most 88%, at most 89%, at most 90%, at most 91%, at most 92%, at most 93%, at most 94%, at most 95%, at most 96%, at most 97%, at most 98%, at most 99%, or is 100% identical, including all values in between, to one or more of SEQ ID NOs: 15, 30, 39, 46-49, 51, 52, 54-59, 63, 66, 68, 70, 71, 73, 76, 78, 82, 83, 86-91, 102, 104, 105, 108, 113-115, 137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189 or to any TS disclosed in this application. In some embodiments, a CBCAS comprises a sequence that is 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, including all values in between, to one or more of SEQ ID NOs: 15, 30, 39, 46-49, 51, 52, 54-59, 63, 66, 68, 70, 71, 73, 76, 78, 82, 83, 86-91, 102, 104, 105, 108, 113-115, 137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189 or to any TS disclosed in this application.

In some embodiments, a CBCAS comprises a sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more SEQ ID NOs: 15, 30, 39, 46-49, 51, 52, 54-59, 63, 66, 68, 70, 71, 73, 76, 78, 82, 83, 86-91, 102, 104, 105, 108, 113-115, 137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189, wherein the sequence is linked to one or more signal peptides. In some embodiments, a signal peptide that comprises SEQ ID NO: 16 or a sequence that has no more than two amino acid substitutions, insertions, additions, or deletions relative to the sequence of SEQ ID NO: 16 is linked to the N-terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 15, 30, 39, 46-49, 51, 52, 54-59, 63, 66, 68, 70, 71, 73, 76, 78, 82, 83, 86-91, 102, 104, 105, 108, 113-115,137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189. In some embodiments, a methionine residue is added to the N-terminus of SEQ ID NO: 16. In some embodiments, a signal peptide that comprises SEQ ID NO: 17 or a sequence that has no more than one amino acid substitution, insertion, addition, or deletion relative to the sequence of SEQ ID NO: 17 is linked to the carboxyl terminus of the sequence that is at least 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID NOs: 15, 30, 39, 46-49, 51, 52, 54-59, 63, 66, 68, 70, 71, 73, 76, 78, 82, 83, 86-91, 102, 104, 105, 108, 113-115, 137-140, 142-143, 145-150, 154, 157, 159, 161, 162, 164, 167, 169, 173, 174, 177-193, 195, 196, 199, 204-206, 322-324, 346, 347, 350, 351-356, 358, 360, 361, 364-406, 408, 409, 410, 423, 432, 453, 455, 460, 464-466, 488, 489, 492-498, 500, 502, 503, 506, 507-548, 550, 551, 552, 565, 574, 595, 597, 602, 698-882, 933, 952-1138, and/or 1189.

In some embodiments, a CBCAS comprises an amino acid deletion or substitution at a residue shown in Table 17, Table 18, or Table 19.

In some embodiments, a CBCAS comprises an amino acid substitution, addition, deletion or insertion at a residue corresponding to position 69, 100, 116, 289, 382, 414, 416, and/or 441 in SEQ ID NO: 13.

In some embodiments, the CBCAS comprises the amino acid Q or R at a residue corresponding to position 69 in SEQ ID NO: 13; the CBCAS comprises the amino acid A at a residue corresponding to position 100 in SEQ ID NO: 13; the CBCAS comprises the amino acid A or G at a residue corresponding to position 116 in SEQ ID NO: 13; the CBCAS comprises the amino acid F or W at a residue corresponding to position 289 in SEQ ID NO: 13; the amino acid S at a residue corresponding to position 382 in SEQ ID NO: 13; the CBCAS comprises the amino acid M or V at a residue corresponding to position 414 in SEQ ID NO: 13; the CBCAS comprises the amino acid F at a residue corresponding to position 416 in SEQ ID NO: 13; and/or the amino acid T or S at a residue corresponding to position 441 in SEQ ID NO: 13.

In some embodiments, a CBCAS comprises an amino acid substitution, addition, deletion or insertion at a residue corresponding to position 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14.

In some embodiments, the CBCAS comprises the amino acid Q at a residue corresponding to position 31 in SEQ ID NO: 14; the amino acid E at a residue corresponding to position 40 in SEQ ID NO: 14; the amino acid Y at a residue corresponding to position 41 in SEQ ID NO: 14; the amino acid P at a residue corresponding to position 46 in SEQ ID NO: 14; the amino acid T at a residue corresponding to position 47 in SEQ ID NO: 14; the amino acid A at a residue corresponding to position 49 in SEQ ID NO: 14; the amino acid F at a residue corresponding to position 51 in SEQ ID NO: 14; the amino acid I at a residue corresponding to position 52 in SEQ ID NO: 14; the amino acid N at a residue corresponding to position 56 in SEQ ID NO: 14; the amino acid S at a residue corresponding to position 58 in SEQ ID NO: 14; the amino acid S at a residue corresponding to position 61 in SEQ ID NO: 14; the amino acid V or L at a residue corresponding to position 63 in SEQ ID NO: 14; the amino acid T at a residue corresponding to position 74 in SEQ ID NO: 14; the amino acid V at a residue corresponding to position 90 in SEQ ID NO: 14; the amino acid G at a residue corresponding to position 95 in SEQ ID NO: 14; the amino acid S at a residue corresponding to position 96 in SEQ ID NO: 14; the amino acid I at a residue corresponding to position 103 in SEQ ID NO: 14; the amino acid S at a residue corresponding to position 116 in SEQ ID NO: 14; the amino acid I at a residue corresponding to position 129 in SEQ ID NO: 14; the amino acid R at a residue corresponding to position 136 in SEQ ID NO: 14; the amino acid E at a residue corresponding to position 143 in SEQ ID NO: 14; the amino acid L at a residue corresponding to position 158 in SEQ ID NO: 14; the amino acid A at a residue corresponding to position 173 in SEQ ID NO: 14, the amino acid A at a residue corresponding to position 181 in SEQ ID NO: 14; the amino acid S at a residue corresponding to position 237 in SEQ ID NO: 14; the amino acid V at a residue corresponding to position 242 in SEQ ID NO: 14; the amino acid R at a residue corresponding to position 247 in SEQ ID NO: 14; the amino acid V at a residue corresponding to position 255 in SEQ ID NO: 14; the amino acid M at a residue corresponding to position 257 in SEQ ID NO: 14; the amino acid E at a residue corresponding to position 268 in SEQ ID NO: 14; the amino acid V at a residue corresponding to position 273 in SEQ ID NO: 14; the amino acid L at a residue corresponding to position 288 in SEQ ID NO: 14; the amino acid F at a residue corresponding to position 290 in SEQ ID NO: 14; the amino acid R at a residue corresponding to position 296 in SEQ ID NO: 14; the amino acid Q at a residue corresponding to position 302 in SEQ ID NO: 14; the amino acid I at a residue corresponding to position 309 in SEQ ID NO: 14; the amino acid S at a residue corresponding to position 311 in SEQ ID NO: 14; the amino acid L at a residue corresponding to position 318 in SEQ ID NO: 14; the amino acid E at a residue corresponding to position 340 in SEQ ID NO: 14; the amino acid Q at a residue corresponding to position 344 in SEQ ID NO: 14; the amino acid L at a residue corresponding to position 345 in SEQ ID NO: 14; the amino acid I at a residue corresponding to position 351 in SEQ ID NO: 14; the amino acid F at a residue corresponding to position 354 in SEQ ID NO: 14; the amino acid Y at a residue corresponding to position 360 in SEQ ID NO: 14; the amino acid D at a residue corresponding to position 361 in SEQ ID NO: 14; the amino acid T at a residue corresponding to position 363 in SEQ ID NO: 14; the amino acid Q at a residue corresponding to position 377 in SEQ ID NO: 14; the amino acid N at a residue corresponding to position 378 in SEQ ID NO: 14; the amino acid A at a residue corresponding to position 379 in SEQ ID NO: 14; the amino acid S at a residue corresponding to position 382 in SEQ ID NO: 14; the amino acid V at a residue corresponding to position 396 in SEQ ID NO: 14; the amino acid V at a residue corresponding to position 411 in SEQ ID NO: 14; the amino acid D at a residue corresponding to position 424 in SEQ ID NO: 14; the amino acid K at a residue corresponding to position 425 in SEQ ID NO: 14; the amino acid T at a residue corresponding to position 430 in SEQ ID NO: 14; the amino acid I at a residue corresponding to position 442 in SEQ ID NO: 14; the amino acid I or V at a residue corresponding to position 443 in SEQ ID NO: 14; the amino acid I at a residue corresponding to position 446 in SEQ ID NO: 14; the amino acid C at a residue corresponding to position 447 in SEQ ID NO: 14; the amino acid L at a residue corresponding to position 459 in SEQ ID NO: 14; the amino acid I at a residue corresponding to position 462 in SEQ ID NO: 14; the amino acid N at a residue corresponding to position 464 in SEQ ID NO: 14; the amino acid I at a residue corresponding to position 465 in SEQ ID NO: 14; the amino acid M at a residue corresponding to position 469 in SEQ ID NO: 14; the amino acid K at a residue corresponding to position 475 in SEQ ID NO: 14; the amino acid M at a residue corresponding to position 479 in SEQ ID NO: 14; the amino acid I at a residue corresponding to position 489 in SEQ ID NO: 14; the amino acid I at a residue corresponding to position 491 in SEQ ID NO: 14; the amino acid N at a residue corresponding to position 492 in SEQ ID NO: 14; the amino acid D at a residue corresponding to position 493 in SEQ ID NO: 14; the amino acid F or P at a residue corresponding to position 494 in SEQ ID NO: 14; the amino acid E or K at a residue corresponding to position 495 in SEQ ID NO: 14; the amino acid N at a residue corresponding to position 496 in SEQ ID NO: 14; the amino acid D at a residue corresponding to position 516 in SEQ ID NO: 14; the amino acid L at a residue corresponding to position 524 in SEQ ID NO: 14; the amino acid D at a residue corresponding to position 528 in SEQ ID NO: 14; the amino acid L or R at a residue corresponding to position 542 in SEQ ID NO: 14; the amino acid R at a residue corresponding to position 543 in SEQ ID NO: 14; and/or the amino acid R at a residue corresponding to position 544 in SEQ ID NO: 14.

In some embodiments, a CBCAS comprises an amino acid substitution, addition, deletion or insertion at a residue corresponding to position 31, 40, 41, 44, 46, 47, 49, 51, 52, 53, 54, 58, 59, 60, 63, 74, 85, 88, 90, 95, 96, 97, 98, 131, 138, 165, 169, 171, 173, 175, 181, 183, 208, 239, 244, 247, 249, 254, 259, 268, 270, 273, 275, 282, 284, 286, 288, 290, 296, 298, 302, 304, 308, 309, 311, 313, 320, 344, 345, 346, 347, 351, 353, 357, 360, 362, 363, 365, 375, 377, 379, 380, 381, 384, 395, 396, 397, 398, 399, 409,411, 415, 424, 425, 426,430, 440, 443, 446, 447, 448, 459, 461, 462, 464, 465, 466, 469, 471, 475, 489, 491, 492, 493, 494, 495, 496, 497, 516, 524, 525, 527, 542, 544, and/or 546 in SEQ ID NO: 20.

In some embodiments, the CBCAS comprises the amino acid R at a residue corresponding to position 31 in SEQ ID NO: 20; the amino acid K or Q at a residue corresponding to position 40 in SEQ ID NO: 20; the amino acid H at a residue corresponding to position 41 in SEQ ID NO: 20; the amino acid T at a residue corresponding to position 44 in SEQ ID NO: 20; the amino acid A or V at a residue corresponding to position 46 in SEQ ID NO: 20; the amino acid T at a residue corresponding to position 47 in SEQ ID NO: 20; the amino acid S or A at a residue corresponding to position 49 in SEQ ID NO: 20; the amino acid L or A at a residue corresponding to position 51 in SEQ ID NO: 20; the amino acid V at a residue corresponding to position 52 in SEQ ID NO: 20; the amino acid L at a residue corresponding to position 53 in SEQ ID NO: 20; the amino acid V at a residue corresponding to position 54 in SEQ ID NO: 20; the amino acid N at a residue corresponding to position 58 in SEQ ID NO: 20; the amino acid F at a residue corresponding to position 59 in SEQ ID NO: 20; the amino acid P at a residue corresponding to position 60 in SEQ ID NO: 20; the amino acid I or L at a residue corresponding to position 63 in SEQ ID NO: 20, the amino acid I at a residue corresponding to position 74 in SEQ ID NO: 20; the amino acid I at a residue corresponding to position 85 in SEQ ID NO: 20; the amino acid L at a residue corresponding to position 88 in SEQ ID NO: 20; the amino acid N at a residue corresponding to position 90 in SEQ ID NO: 20; the amino acid G at a residue corresponding to position 95 in SEQ ID NO: 20; the amino acid T at a residue corresponding to position 96 in SEQ ID NO: 20; the amino acid G at a residue corresponding to position 97 in SEQ ID NO: 20; the amino acid T at a residue corresponding to position 98 in SEQ ID NO: 20; the amino acid I at a residue corresponding to position 131 in SEQ ID NO: 20; the amino acid R at a residue corresponding to position 138 in SEQ ID NO: 20; the amino acid N at a residue corresponding to position 165 in SEQ ID NO: 20; the amino acid L at a residue corresponding to position 169 in SEQ ID NO: 20; the amino acid L at a residue corresponding to position 171 in SEQ ID NO: 20; the amino acid A at a residue corresponding to position 173 in SEQ ID NO: 20; the amino acid A at a residue corresponding to position 175 in SEQ ID NO: 20; the amino acid A at a residue corresponding to position 181 in SEQ ID NO: 20; the amino acid A at a residue corresponding to position 183 in SEQ ID NO: 20; the amino acid Q at a residue corresponding to position 208 in SEQ ID NO: 20; the amino acid S at a residue corresponding to position 239 in SEQ ID NO: 20; the amino acid V at a residue corresponding to position 244 in SEQ ID NO: 20; the amino acid R at a residue corresponding to position 247 in SEQ ID NO: 20; the amino acid R at a residue corresponding to position 249 in SEQ ID NO: 20; the amino acid M at a residue corresponding to position 254 in SEQ ID NO: 20; the amino acid M at a residue corresponding to position 259 in SEQ ID NO: 20; the amino acid E at a residue corresponding to position 268 in SEQ ID NO: 20; the amino acid E at a residue corresponding to position 270 in SEQ ID NO: 20; the amino acid V at a residue corresponding to position 273 in SEQ ID NO: 20; the amino acid V at a residue corresponding to position 275 in SEQ ID NO: 20; the amino acid M at a residue corresponding to position 282 in SEQ ID NO: 20; the amino acid E at a residue corresponding to position 284 in SEQ ID NO: 20; the amino acid E at a residue corresponding to position 286 in SEQ ID NO: 20; the amino acid L at a residue corresponding to position 288 in SEQ ID NO: 20; the amino acid F or L at a residue corresponding to position 290 in SEQ ID NO: 20; the amino acid R at a residue corresponding to position 296 in SEQ ID NO: 20; the amino acid Q at a residue corresponding to position 302 in SEQ ID NO: 20; the amino acid Q at a residue corresponding to position 304 in SEQ ID NO: 20; the amino acid T at a residue corresponding to position 308 in SEQ ID NO: 20; the amino acid K or I at a residue corresponding to position 309 in SEQ ID NO: 20; the amino acid S or I at a residue corresponding to position 311 in SEQ ID NO: 20; the amino acid S at a residue corresponding to position 313 in SEQ ID NO: 20; the amino acid L at a residue corresponding to position 320 in SEQ ID NO: 20; the amino acid Q at a residue corresponding to position 344 in SEQ ID NO: 20; the amino acid L at a residue corresponding to position 345 in SEQ ID NO: 20; the amino acid Q at a residue corresponding to position 346 in SEQ ID NO: 20; the amino acid L at a residue corresponding to position 347 in SEQ ID NO: 20; the amino acid I at a residue corresponding to position 351 in SEQ ID NO: 20; the amino acid I at a residue corresponding to position 353 in SEQ ID NO: 20; the amino acid L at a residue corresponding to position 357 in SEQ ID NO: 20; the amino acid Y at a residue corresponding to position 360 in SEQ ID NO: 20; the amino acid Y at a residue corresponding to position 362 in SEQ ID NO: 20; the amino acid T or D at a residue corresponding to position 363 in SEQ ID NO: 20; the amino acid T at a residue corresponding to position 365 in SEQ ID NO: 20; the amino acid G at a residue corresponding to position 375 in SEQ ID NO: 20; the amino acid R at a residue corresponding to position 377 in SEQ ID NO: 20, the amino acid Q or A at a residue corresponding to position 379 in SEQ ID NO: 20; the amino acid N at a residue corresponding to position 380 in SEQ ID NO: 20; the amino acid A at a residue corresponding to position 381 in SEQ ID NO: 20; the amino acid K at a residue corresponding to position 384 in SEQ ID NO: 20; the amino acid S at a residue corresponding to position 395 in SEQ ID NO: 20; the amino acid V at a residue corresponding to position 396 in SEQ ID NO: 20; the amino acid F at a residue corresponding to position 397 in SEQ ID NO: 20; the amino acid V at a residue corresponding to position 398 in SEQ ID NO: 20; the amino acid Q at a residue corresponding to position 399 in SEQ ID NO: 20; the amino acid I at a residue corresponding to position 409 in SEQ ID NO: 20; the amino acid A at a residue corresponding to position 411 in SEQ ID NO: 20; the amino acid A at a residue corresponding to position 415 in SEQ ID NO: 20; the amino acid D at a residue corresponding to position 424 in SEQ ID NO: 20; the amino acid K at a residue corresponding to position 425 in SEQ ID NO: 20; the amino acid at a residue corresponding to position in SEQ ID NO: 20; the amino acid D at a residue corresponding to position 426 in SEQ ID NO: 20; the amino acid T at a residue corresponding to position 430 in SEQ ID NO: 20; the amino acid L at a residue corresponding to position 440 in SEQ ID NO: 20; the amino acid V at a residue corresponding to position 443 in SEQ ID NO: 20; the amino acid I at a residue corresponding to position 446 in SEQ ID NO: 20; the amino acid C at a residue corresponding to position 447 in SEQ ID NO: 20; the amino acid I at a residue corresponding to position 448 in SEQ ID NO: 20; the amino acid L at a residue corresponding to position 459 in SEQ ID NO: 20; the amino acid L at a residue corresponding to position 461 in SEQ ID NO: 20; the amino acid I at a residue corresponding to position 462 in SEQ ID NO: 20; the amino acid N or I at a residue corresponding to position 464 in SEQ ID NO: 20; the amino acid I at a residue corresponding to position 465 in SEQ ID NO: 20; the amino acid N at a residue corresponding to position 466 in SEQ ID NO: 20; the amino acid M at a residue corresponding to position 469 in SEQ ID NO: 20; the amino acid M at a residue corresponding to position 471 in SEQ ID NO: 20; the amino acid K at a residue corresponding to position 475 in SEQ ID NO: 20; the amino acid I at a residue corresponding to position 489 in SEQ ID NO: 20; the amino acid I at a residue corresponding to position 491 in SEQ ID NO: 20; the amino acid N at a residue corresponding to position 492 in SEQ ID NO: 20; the amino acid D at a residue corresponding to position 493 in SEQ ID NO: 20; the amino acid P or N at a residue corresponding to position 494 in SEQ ID NO: 20; the amino acid K at a residue corresponding to position 495 in SEQ ID NO: 20; the amino acid N at a residue corresponding to position 496 in SEQ ID NO: 20; the amino acid K at a residue corresponding to position 497 in SEQ ID NO: 20; the amino acid D at a residue corresponding to position 516 in SEQ ID NO: 20; the amino acid L at a residue corresponding to position 524 in SEQ ID NO: 20; the amino acid V at a residue corresponding to position 525 in SEQ ID NO: 20; the amino acid V at a residue corresponding to position 527 in SEQ ID NO: 20; the amino acid R at a residue corresponding to position 542 in SEQ ID NO: 20; the amino acid R at a residue corresponding to position 544 in SEQ ID NO: 20; and/or the amino acid R at a residue corresponding to position 546 in SEQ ID NO: 20.

In some embodiments, the CBCAS comprises any combination of amino acid substitutions shown in Table 17, Table 18, or Table 19.

In some embodiments, a CBCAS comprises relative to SEQ ID NO: 14: R31Q, K40Q, H41Y, H56N, M61S, I74T, N90V, V129I, S255V, V288L, M290F, K296R, T340E, F345L, T3511, F360Y, A411V, E424D, Q475K, T492N, H494P, and A495E; R31Q, K40Q, H41Y, V46P, H56N, Q58S, M61S, 174T, N90V, V1291, S255V, V288L, M290F, K296R, T340E, F345L, F360Y, A411V, E424D, T446L, Q475K, T492N, H494P, and A495E; R31Q, K40Q, H41Y, V46P, M61S, I74T, N90V, V1291, S255V, V288L, M290F, K296R, T340E, F345L, F360Y, A411V, E424D, T492N, H494P, and A495E; R31Q, K40Q, H41Y, V46P, M61S, 174T, N90V, V129I, S255V, V288L, M290F, K296R, T340E, F345L, F360Y, A411V, E424D, T492N, H494P, and A495E; R31Q, K40Q, H41Y, V46P, H56N, Q58S, M61S, I74T, N90V, V1291, V158L, S255V, V288L, M290F, K296R, T340E, F345L, F360Y, A411V, E424D, Q475K, T492N, H494P, and A495E; R31Q, K40Q, H41Y, Q58S, M61S, 174T, N90V, V129I, H143E, S255V, V288L, M290F, K296R, T340E, F345L, F360Y, A411V, E424D, Q475K, T492N, H494P, and A495E; R31Q, K40Q, H41Y, M61 S, 174T, N90V, V129I, S255V, V288L, M290F, K296R, T340E, F345L, F360Y, A411V, E424D, Q475K, T492N, H494P, and A495E; R31Q, K40Q, H41Y, V46P, H56N, M61S, 174T, N90V, V1291, H143E, S255V, V288L, M290F, K296R, T340E, F345L, Y354F, F360Y, A411V, E424D, Q475K, T492N, H494P, and A495E; R31Q, K40Q, H41Y, H56N, Q58S, M61S, I74T, N90V, V1291, S255V, V288L, M290F, K296R, T340E, F345L, F360Y, A411V, E424D, Q475K, T492N, H494P, and A495E; R31Q, K40Q, H41Y, 174T, N90V, V129I, H143E, S255V, V288L, M290F, K296R, F345L, F360Y, A411V, E424D, T492N, H494P, and A495E; or R31Q, K40Q, H41Y, V46P, V52I, H56N, Q58S, M61 S, I74T, N90V, V1291, S255V, V288L, M290F, K296R, T340E, F345L, F360Y, A411V, E424D, Q475K, T492N, H494P, and A495E.

In some embodiments, a CBCAS comprises relative to SEQ ID NO: 13: T47A, L49P, N56H, N57D, P58Q, H69Q, H89N, and G95A.

In some embodiments, a CBCAS comprises relative to SEQ ID NO: 14: R31Q, K40Q, H41Y, H56N, M61S, 174T, N90V, V129I, S255V, V288L, M290F, K296R, T340E, F345L, T351I, F360Y, A411V, E424D, Q475K, T492N, H494P, and A495E; R31Q, K40Q, H41 Y, V46P, H56N, Q58S, M61S, 174T, N90V, V1291, S255V, V288L, M290F, K296R, T340E, F345L, F360Y, A411V, E424D, T446I, Q475K, T492N, H494P, and A495E; R31Q, K40Q, H41Y, V46P, M61S, I74T, N90V, V1291, S255V, V288L, M290F, K296R, T340E, F345L, F360Y, A411V, E424D, T492N, H494P, and A495E; R31Q, K40Q, H41Y, V46P, H56N, M61S, I74T, N90V, V129L, S255V, V288L, M290F, K296R, F345L, F360Y, A411V, E424D, T492N, H494P, and A495E; R31Q, K40Q, H41Y, V46P, H56N, Q58S, M61S, 174T, N90V, V129I, V158L. S255V, V288L, M290F, K296R, T340E, F345L, F360Y, A411V, E424D, Q475K, T492N, H494P, and A495E; R31Q, K40Q, H41Y, Q58S, M61S, I74T, N90V, V1291, H143E, S255V, V288L, M290F, K296R, T340E, F345L, F360Y, A411V, E424D, Q475K, T492N, H494P, and A495E; R31 Q, K40Q, H41Y, M61S, I74T, N90V, V1291, S255V, V288L, M290F, K296R, T340E, F345L, F360Y, A411V, E424D, Q475K, T492N, H494P, and A495E; R31Q, K40Q, H41Y, V46P, H56N, M61S, I74T, N90V, V129I, H143E, S255V, V288L, M290F, K296R, T340E, F345L, Y354F, F360Y, A411V, E424D, Q475K, T492N, H494P, and A495E; R31Q, K40Q, H41Y, H56N, Q58S, M61S, I74T, N90V, V129I, S255V, V288L, M290F, K296R, T340E, F345L, F360Y, A411V, E424D, Q475K, T492N, H494P, and A495E; or R31Q, K40Q, H41Y, V46P, V52I, H56N, Q58S, M61S, I74T, N90V, V129I, S255V, V288L, M290F, K296R, T340E, F345L, F360Y, A411V, E424D, Q475K, T492N, H494P, and A495E.

In some embodiments, a CBCAS comprises relative to SEQ ID NO: 14: Q58S, V288L, and F345L; R31Q, V52I, H56N, Q58S, M61S, I74T, N90V, A250P, S255V, F345L, Q475K, and T492N; R31Q, H56N, 174T, N90V, H143E, A250P, S255V, Q475K, and T492N; R31Q, H56N, I74T, N90V, A250P, S255V, L443I, Q475K, and T492N; H56N, M61S, N90V, A250D, S255V, V288L, Q475K, T492N, and A495E; R31Q, H56N, I74T, N90V, K215R, A250P, S255V, Q475K, and T492N; R31Q, P49A, H56N, Q58S, M61S, I74T, N90V, H143E, A250P, S255V, V288L, F345L, Q475K, and T492N; R31Q, A47T, H56N, I74T, N90V, A250P, S255V, Q475K, and T492N; M61S, N90V, A250D, S255V, Q475K, T492N, A495E, and N498T; R31Q, H56N, M61S, I74T, N89H, N90V, S100A, H136R, E150Q, N196K, N211D, A250P, S255V, V288M, F345M, S382K, L443I, Q475K, and T492N; R31Q, H56N, Q58S, M61S, I74T, N90V, H143E, A250P, S255V, V288L, T340E, F345L, E424D, Q475K, and T492N; R31Q, H56N, I74T, S88L, N90V, A250P, S255V, Q475K, and T492N; R31Q, V52I, H56N, Q58S, M61S, I74T, N90V, H143E, A250P, S255V, V288L, F345L, Q475K, and T492N; R31Q, H56N, Q58S, M61S, 174T, N90V, H143E, A250P, S255V, V288L, T340E, F345L, E424D, Q475K, and T492N; R31Q, H56N, Q58S, M61S, I74T, N90V, H143E, A250P, S255V, V288L, F345L, A411V, Q475K, and T492N; R31Q, V52I, H56N, Q58S, M61S, I74T, N90V, A250P, S255V, V288L, F345L, Q475K, and T492N; R31Q, K50L, H56N, I74T, N90V, A250P, S255V, Q475K, and T492N; R31Q, A47T, V52I, H56N, Q58S, M61S, I74T, N90V, H143E, A250P, S255V, V288L, T340E, F345L, Q475K, and T492N; or R31Q, H56N, M61S, I74T, N89H, N90V, S100A, N196K, N211D, A250P, S255V, I257R, V288M, F345M, S382K, L443I, Q475K, and T492N.

In some embodiments, a CBCAS comprises an amino acid insertion at a residue corresponding to position 253 in SEQ ID NO: 13. In some embodiments, the amino acid S is inserted at a residue corresponding to position 253 in SEQ ID NO: 13.

In some embodiments, one or more amino acid substitutions in a CBCAS causes a shift in product profile from THCA to CBCA, as compared to a CBCAS without such a substitution. In some embodiments, the amino acid substitution is at a residue corresponding to position 158 relative to SEQ ID NO: 14. In some embodiments, the amino acid substitution is V158L relative to SEQ ID NO: 14.

In some embodiments, one or more amino acid substitutions increases the substrate selectivity of the CBCAS such as the selectivity for a compound of Formula (8), CBGA, CBGVA or a combination thereof, as compared to a CBCAS without such a substitution.

In some embodiments, one or more amino acid substitutions increases the product specificity of the CBCAS, such as the specificity for a compound of Formula (11), CBCA, CBCVA or a combination thereof, as compared to a CBCAS without such a substitution. In some embodiments, one or more amino acid substitutions increases the product specificity of the CBCAS for CBCVA. In some embodiments, the amino acid substitution is at a residue corresponding to position 446 relative to SEQ ID NO: 14, a position that is predicted to be within 4 angstroms of the substrate binding site of the CBCAS. In some embodiments, the amino acid substitution is T4461 relative to SEQ ID NO: 14, which alters the residue from an uncharged polar residue to a bulkier hydrophobic residue.

In some embodiments, a CBCAS comprises one or more amino acid substitutions that alter the secondary or tertiary structure of the CBCAS, as compared to a CBCAS without such a substitution. In some embodiments, one or more amino acid substitutions are close to the active site. In some embodiments, the one or more amino acid substitutions are Y354F and/or A411V relative to SEQ ID NO:14.

### Additional Cannabinoid Pathway Enzymes

Methods for production of cannabinoids and cannabinoid precursors can further include expression of one or more of: an acyl activating anzyme (AAE); a polyketide synthase (PKS) (e.g., OLS); a polykeide cyclase (PKC); and a prenyltransferase (PT).

### Acyl Activating Enzyme (AAE)

A host cell described in this disclosure may comprise an AAE. As used in this disclosure, an AAE refers to an enzyme that is capable of catalyzing the esterification between a thiol and a substrate (e.g., optionally substituted aliphatic or aryl group) that has a carboxylic acid moiety. In some embodiments, an AAE is capable of using Formula (1):

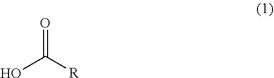

or a salt, solvate, hydrate, polymorph, co-crystal, tautomer, stereoisomer, isotopically labeled derivative thereof to produce a product of Formula (2):

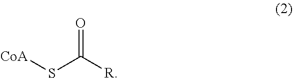

R is as defined in this application. In certain embodiments, R is hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C2-10 alkyl, optionally substituted C10-C20 alkyl, optionally substituted C20-C30 alkyl, optionally substituted C30-C40 alkyl, or optionally substituted C40-C50 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C20 branched alkyl. In certain embodiments, R is optionally substituted C1-C20 alkyl, optionally substituted C1-C10 alkyl, optionally substituted C10-C20 alkyl, optionally substituted C20-C30 alkyl, optionally substituted C30-C40 alkyl, or optionally substituted C40-C50 alkyl. In certain embodiments, R is optionally substituted C1-C10 alkyl. In certain embodiments, R is optionally substituted C3 alkyl. In certain embodiments, R is optionally substituted n-propyl. In certain embodiments, R is unsubstituted n-propyl. In certain embodiments, R is optionally substituted C1-C8 alkyl. In some embodiments, R is a C2-C6 alkyl. In certain embodiments, R is optionally substituted C1-C5 alkyl. In certain embodiments, R is optionally substituted C3-C5 alkyl. In certain embodiments, R is optionally substituted C3 alkyl. In certain embodiments, R is optionally substituted C5 alkyl. In certain embodiments, R is of formula:

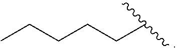 [SMILES: *CCCCC]

In certain embodiments, R is of formula:

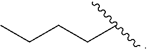 [SMILES: *CCCC]

In certain embodiments, R is of formula:

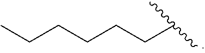 [SMILES: *CCCCCC]

In certain embodiments, R is of formula:

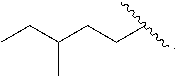 [SMILES: *CCC(C)CC]

In certain embodiments, R is optionally substituted propyl. In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

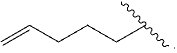 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

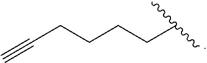 [SMILES: *CCCCC#C]

In certain embodiments, R is optionally substituted carbocyclyl. In certain embodiments, R is optionally substituted aryl (e.g., phenyl or napthyl).

In some embodiments, a substrate for an AAE is produced by fatty acid metabolism within a host cell. In some embodiments, a substrate for an AAE is provided exogenously.

In some embodiments, an AAE is capable of catalyzing the formation of hexanoyl-coenzyme A (hexanoyl-CoA) from hexanoic acid and coenzyme A (CoA). In some embodiments, an AAE is capable of catalyzing the formation of butanoyl-coenzyme A (butanoyl-CoA) from butanoic acid and coenzyme A (CoA).

As one of ordinary skill in the art would appreciate, an AAE could be obtained from any source, including naturally occurring sources and synthetic sources (e.g., a non-naturally occurring AAE). In some embodiments, an AAE is a Cannabis enzyme. Non-limiting examples of AAEs include C. sativa hexanoyl-CoA synthetase 1 (CsHCS1) and C. sativa hexanoyl-CoA synthetase 2 (CsHCS2) as disclosed in U.S. Pat. No. 9,546,362, which is incorporated by reference in this application in its entirety.

CsHCS1 has the sequence:

(SEQ ID NO: 5)
MGKNYKSLDSVVASDFIALGITSEVAETLHGRLAEIVCNYGAATPQTWIN
IANHILSPDLPFSLHQMLFYGCYKDFGPAPPAWIPDPEKVKSTNLGALLE
KRGKEFLGVKYKDPISSFSHFQEFSVRNPEVYWRTVLMDEMKISFSKDPE
CILRRDDINNPGGSEWLPGGYLNSAKNCLNVNSNKKLNDTMIVWRDEGND
DLPLNKLTLDQLRKRVWLVGYALEEMGLEKGCAIAIDMPMHVDAVVIYLA
IVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQ
PVDAYTNILFSSGTTGEPKAIPWTQATPLKAAADGWSHLDIRKGDVIVWP
TNLGWMMGPWLVYASLLNGASIALYNGSPLVSGFAKFVQDAKVTMLGVVP
SIVRSWKSTNCVSGYDWSTIRCFSSSGEASNVDEYLWLMGRANYKPVIEM
CGGTEIGGAFSAGSFLQAQSLSSFSSQCMGCTLYILDKNGYPMPKNKPGI
GELALGPVMFGASKTLLNGNHHDVYFKGMPTLNGEVLRRHGDIFELTSNG
YYHAHGRADDTMNIGGIKISSIEIERVCNEVDDRVFETTAIGVPPLGGGP
EQLVIFFVLKDSNDTTIDLNQLRLSENLGLQKKLNPLFKVTRVVPLSSLP
RTATNKIMRRVLRQFSHFE.

CsHCS2 has the sequence:

(SEQ ID NO: 6)
MEKSGYGRDGIYRSLRPPLHLPNNNNLSMVSFLERNSSSYPQKPALIDSE
TNQILSFSHFKSTVIKVSHGFLNLGIKKNDVVLIYAPNSIHFPVCFLGII
ASGAIATTSNPLYTVSELSKQVKDSNPKLIITVPQLLEKVKGFNLPTILI
GPDSEQESSSDKVMTENDLVNLGGSSGSEFPIVDDFKQSDTAALLYSSGT
TGMSKGVVLTHKNFIASSLMVTMEQDLVGEMDNVFLCFLPMFHVFGLAII
TYAQLQRGNTVISMARFDLEKMLKDVEKYKVTHLWVVPPVILALSKNSMV
KKFNLSSIKYIGSGAAPLGKDLMEECSKVVPYGIVAQGYGMTETCGIVSM
EDIRGGKRNSGSAGMLASGVEAQIVSVDTLKPLPPNQLGEIWVKGPNMMQ
GYFNNPQATKLTIDKKGWVHTGDLGYFDEDGHLYVVDRIKELIKYKGFQV
APAELEGLLVSHPEILDAVVIPFPDAEAGEVPVAYVVRSPNSSLTENDVK
KFIAGQVASFKRLRKVTFINSVPKSASGKILRRELIQKVRSNM.

### Polyketide Synthases (PKS)

A host cell described in this application may comprise a PKS. As used in this application, a “PKS” refers to an enzyme that is capable of producing a polyketide. In certain embodiments, a PKS converts a compound of Formula (2) to a compound of Formula (4), (5), and/or (6). In certain embodiments, a PKS converts a compound of Formula (2) to a compound of Formula (4). In certain embodiments, a PKS converts a compound of Formula (2) to a compound of Formula (5). In certain embodiments, a PKS converts a compound of Formula (2) to a compound of Formula (4) and/or (5). In certain embodiments, a PKS converts a compound of Formula (2) to a compound of Formula (5) and/or (6).

In some embodiments, a PKS is a tetraketide synthase (TKS). In certain embodiments, a PKS is an olivetol synthase (OLS). As used in this application, an “OLS” refers to an enzyme that is capable of using a substrate of Formula (2a) to form a compound of Formula (4a), (5a) or (6a) as shown in FIG. 1.

In certain embodiments, a PKS is a divarinic acid synthase (DVS).

In certain embodiments, polyketide synthases can use hexanoyl-CoA or any acyl-CoA (or a product of Formula (2):

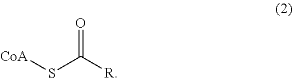

and three malonyl-CoAs as substrates to form 3,5,7-trioxododecanoyl-CoA or other 3,5,7-trioxo-acyl-CoA derivatives; or to form a compound of Formula (4):

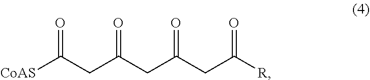

wherein R is hydrogen, optionally substituted acyl, optionally substituted alkyl, optionally substituted alkenyl, optionally substituted alkynyl, optionally substituted carbocyclyl, or optionally substituted aryl; depending on substrate. R is as defined in this application. In some embodiments, R is a C2-C6 optionally substituted alkyl. In some embodiments, R is a propyl or pentyl. In some embodiments, R is pentyl. In some embodiments, R is propyl. A PKS may also bind isovaleryl-CoA, octanoyl-CoA, hexanoyl-CoA, and butyryl-CoA. In some embodiments, a PKS is capable of catalyzing the formation of a 3,5,7-trioxoalkanoyl-CoA (e.g. 3,5,7-trioxododecanoyl-CoA). In some embodiments, an OLS is capable of catalyzing the formation of a 3,5,7-trioxoalkanoyl-CoA (e.g. 3,5,7-trioxododecanoyl-CoA).

In some embodiments, a PKS uses a substrate of Formula (2) to form a compound of Formula (4):

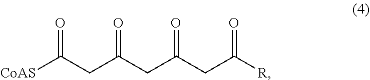

wherein R is unsubstituted pentyl.

As one of ordinary skill in the art would appreciate a PKS, such as an OLS, could be obtained from any source, including naturally occurring sources and synthetic sources (e.g., a non-naturally occurring PKS). In some embodiments a PKS is from Cannabis. In some embodiments a PKS is from Dictyostelium. Non-limiting examples of PKS enzymes may be found in U.S. Pat. No. 6,265,633; PCT Publication No. WO2018/148848 A1; PCT Publication No. WO2018/148849 A1: and U.S. Patent Publication No. 2018/155748, and WO 2020/176547, which are incorporated by reference in this application in their entireties.

A non-limiting example of an OLS is provided by UniProtKB—B1Q2B6 from C. sativa. In C. sativa, this OLS uses hexanoyl-CoA and malonyl-CoA as substrates to form 3,5,7-trioxododecanoyl-CoA. OLS (e.g., UniProtKB—B1Q2B6) in combination with olivetolic acid cyclase (OAC) produces olivetolic acid (OA) in C. sativa.

The amino acid sequence of UniProtKB—B1Q2B6 is:

(SEQ ID NO: 7)
MNHLRAEGPASVLAIGTANPENILLQDEFPDYYFRVTKSEHMTQLKEKFR
KICDKSMIRKRNCFLNEEHLKQNPRLVEHEMQTLDARQDMLVVEVPKLGK
DACAKAIKEWGQPKSKITHLIFTSASTTDMPGADYHCAKLLGLSPSVKRV
MMYQLGCYGGGTVLRIAKDIAENNKGARVLAVCCDIMACLFRGPSESDLE
LLVGQAIFGDGAAAVIVGAEPDESVGERPIFELVSTGQTILPNSEGTIGG
HIREAGLIFDLHKDVPMLISNNIEKCLIEAFTPIGISDWNSIFWITHPGG
KAILDKVEEKLHLKSDKFVDSRHVLSEHGNMSSSTVLFVMDELRKRSLEE
GKSTTGDGFEWGVLFGFGPGLTVERVVVRSVPIKY.

PKS enzymes described in this application may or may not have cyclase activity. In some embodiments where the PKS enzyme does not have cyclase activity, one or more exogenous polynucleotides that encode a polyketide cyclase (PKC) enzyme may also be co-expressed in the same host cells to enable conversion of hexanoic acid or butyric acid or other fatty acid conversion into olivetolic acid or divarinolic acid or other precursors of cannabinoids. In some embodiments, the PKS enzyme and a PKC enzyme are expressed as separate distinct enzymes. In some embodiments, a PKS enzyme that lacks cyclase activity and a PKC are linked as part of a fusion polypeptide that is a bifunctional PKS. In some embodiments, a bifunctional PKC is referred to as a bifunctional PKS-PKC. In some embodiments, a bifunctional PKC is a bifunctional tetraketide synthase (TKS-TKC). As used in this application, a bifunctional PKS is an enzyme that is capable of producing a compound of Formula (6):

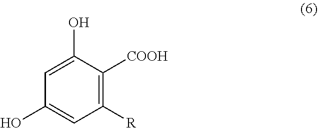

from a compound of Formula (2):

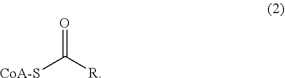

and a compound of Formula (3):

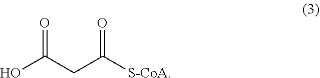

In some embodiments, a PKS produces more of a compound of Formula (6):

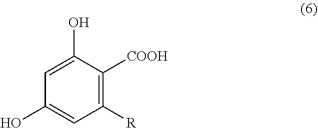

as compared to a compound of Formula (5):

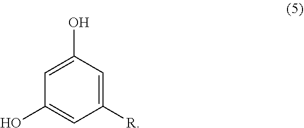

As a non-limiting example, a compound of Formula (6):

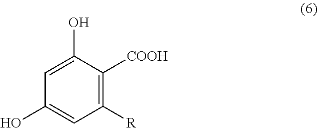

is olivetolic acid (Formula (6a)):

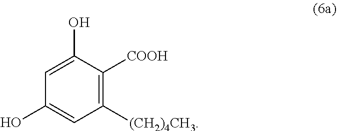

As a non-limiting example, a compound of Formula (5):

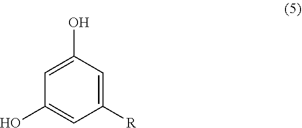

is olivetol (Formula (5a)):

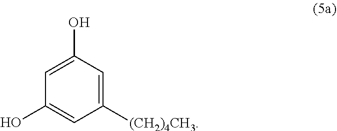

In some embodiments, a polyketide synthase of the present disclosure is capable of catalyzing a compound of Formula (2):

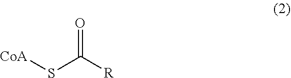

and a compound of Formula (3):

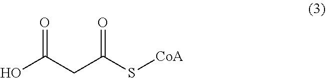

to produce a compound of Formula (4):

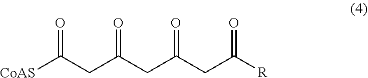

and also further catalyzes a compound of Formula (4):

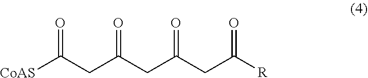

to produce a compound of Formula (6):

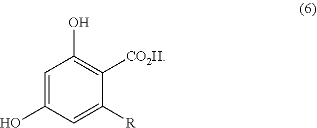

In some embodiments, the PKS is not a fusion protein. In some embodiments, a PKS that is capable of catalyzing a compound of Formula (2):

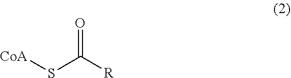

and a compound of Formula (3):

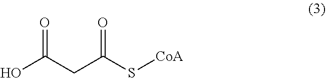

to produce a compound of Formula (4):

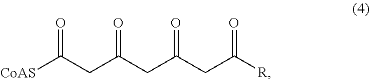

and is also capable of further catalyzing the production of a compound of Formula (6):

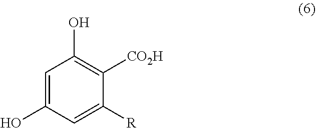

from the compound of Formula (4):

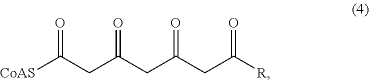

is preferred because it avoids the need for an additional polyketide cyclase to produce a compound of Formula (6):

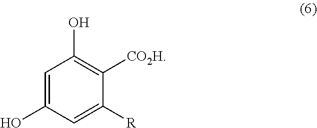

In some embodiments, such an enzyme that is a bifunctional PKS eliminates the transport considerations needed with addition of a polyketide cyclase, whereby the compound of Formula (4), being the product of the PKS, must be transported to the PKS for use as a substrate to be converted into the compound of Formula (6).

In some embodiments, a PKS is capable of producing olivetolic acid in the presence of a compound of Formula (2a):

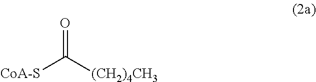

### and Formula (3a):

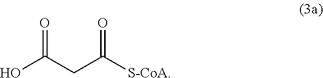

In some embodiments, an OLS is capable of producing olivetolic acid in the presence of a compound of Formula (2a):

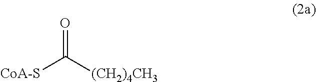

### and Formula (3a):

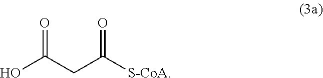

### Polyketide Cyclase (PKC)

A host cell described in this disclosure may comprise a PKC. As used in this application, a “PKC” refers to an enzyme that is capable of cyclizing a polyketide.

In certain embodiments, a polyketide cyclase (PKC) catalyzes the cyclization of an oxo fatty acyl-CoA (e.g., a compound of Formula (4):

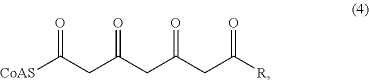

or 3,5,7-trioxododecanoyl-COA, 3,5,7-trioxodecanoyl-COA) to the corresponding intramolecular cyclization product (e.g., compound of Formula (6), including olivetolic acid and divarinic acid). In some embodiments, a PKC catalyzes the formation of a compound which occurs in the presence of a PKS. PKC substrates include trioxoalkanol-CoA, such as 3,5,7-Trioxododecanoyl-CoA, or a compound of Formula (4):

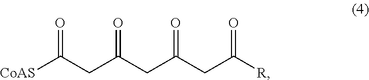

wherein R is hydrogen, optionally substituted acyl, optionally substituted alkyl, optionally substituted alkenyl, optionally substituted alkynyl, optionally substituted carbocyclyl, or optionally substituted aryl. In certain embodiments, a PKC catalyzes a compound of Formula (4):

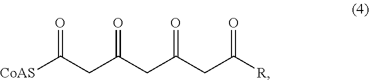

wherein R is hydrogen, optionally substituted acyl, optionally substituted alkyl, optionally substituted alkenyl, optionally substituted alkynyl, optionally substituted carbocyclyl, or optionally substituted aryl; to form a compound of Formula (6):

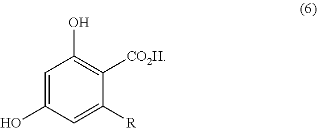

wherein R is hydrogen, optionally substituted acyl, optionally substituted alkyl, optionally substituted alkenyl, optionally substituted alkynyl, optionally substituted carbocyclyl, or optionally substituted aryl; as substrates. R is as defined in this application. In some embodiments, R is a C2-C6 optionally substituted alkyl. In some embodiments, R is a propyl or pentyl. In some embodiments, R is pentyl. In some embodiments, R is propyl. In certain embodiments, a PKC is an olivetolic acid cyclase (OAC). In certain embodiments, a PKC is a divarinic acid cyclase (DAC).

As one of ordinary skill in the art would appreciate a PKC could be obtained from any source, including naturally occurring sources and synthetic sources (e.g., a non-naturally occurring PKC). In some embodiments, a PKC is from Cannabis. Non-limiting examples of PKCs include those disclosed in U.S. Pat. Nos. 9,611,460; 10,059,971; U.S. Patent Publication No. 2019/0169661, and PCT Application No. PCT/US21/37954, which are incorporated by reference in this application in their entireties.

In some embodiments, a PKC is an OAC. As used in this application, an “OAC” refers to an enzyme that is capable of catalyzing the formation of olivetolic acid (OA). In some embodiments, an OAC is an enzyme that is capable of using a substrate of Formula (4a)(3,5,7-trioxododecanoyl-CoA):

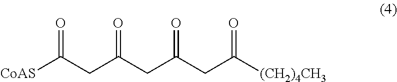

to form a compound of Formula (6a) (olivetolic acid):

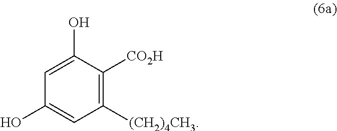

Olivetolic acid cyclase from C. sativa (CsOAC) is a 101 amino acid enzyme that performs non-decarboxylative cyclization of the tetraketide product of olivetol synthase (FIG. 4 Structure 4a) via aldol condensation to form olivetolic acid (FIG. 4 Structure 6a). CsOAC was identified and characterized by Gagne et al. (PNAS 2012) via transcriptome mining, and its cyclization function was recapitulated in vitro to demonstrate that CsOAC is required for formation of olivetolic acid in C. sativa. A crystal structure of the enzyme was published by Yang et al. (FEBS J. 2016 March; 283(6):1088-106), which revealed that the enzyme is a homodimer and belongs to the α+β barrel (DABB) superfamily of protein folds. CsOAC is the only known plant polyketide cyclase. Multiple fungal Type III polyketide synthases have been identified that perform both polyketide synthase and cyclization functions (Funa et al., J Biol Chem. 2007 May 11; 282(19):14476-81); however, in plants such a dual function enzyme has not yet been discovered.

A non-limiting example of an amino acid sequence of an OAC in C. sativa is provided by UniProtKB—I6WU39 (SEQ ID NO: 1), which catalyzes the formation of olivetolic acid (OA) from 3,5,7-Trioxododecanoyl-CoA.

The sequence of UniProtKB—16WU39 (SEQ ID NO: 1) is:

MAVKHLIVLKFKDEITEAQKEEFFKTYVNLVNIIPAMKDVYWGKDVTQKN
KEEGYTHIVEVTFESVETIQDYIIHPAHVGFGDVYRSFWEKLLIFDYTPR
K.

A non-limiting example of a nucleic acid sequence encoding C. sativa OAC is:

(SEQ ID NO: 2)
atggcagtgaagcatttgattgtattgaagttcaaagatgaaatcacaga
agcccaaaaggaagaatttttcaagacgtatgtgaatcttgtgaatatca
tcccagccatgaaagatgtatactggggtaaagatgtgactcaaaagaat
aaggaagaagggtacactcacatagttgaggtaacatttgagagtgtgga
gactattcaggactacattattcatcctgcccatgttggatttggagatg
tctatcgttctttctgggaaaaacttctcatttttgactacacaccacga
aag.

### Prenyltransferase (PT)

A host cell described in this application may comprise a prenyltransferase (PT). As used in this application, a “PT” refers to an enzyme that is capable of transferring prenyl groups to acceptor molecule substrates. Non-limiting examples of prenyltransferases are described in in U.S. Pat. No. 7,544,498 and Kumano et al., Boorg Med Chem. 2008 Sep. 1; 16(17): 8117-8126 (e.g., NphB), PCT Publication No. WO2018/200888 (e.g., CsPT4), U.S. Pat. No. 8,884,100 (e.g., CsPT1); Canadian Patent No. CA2718469; Valliere et al., Nat Commun. 2019 Feb. 4; 10(1):565 (e.g., NphB variants); PCT Publication Nos: WO2019/173770, WO2019/183152, and WO2020/210810 (e.g., NphB variants); Luo et al., Nature 2019 March; 567(7746):123-126 (e.g., CsPT4); WO 2021/034848; U.S. 63/091,292 and U.S. 63/188,442 (e.g., CsPT variants and chimeras), which are incorporated by reference in their entireties. In some embodiments, a PT is capable of producing cannabigerolic acid (CBGA), cannabigerovarinic acid (CBGVA), or other cannabinoids or cannabinoid-like substances. In some embodiments, a PT is cannabigerolic acid synthase (CBGAS). In some embodiments, a PT is cannabigerovarinic acid synthase (CBGVAS).

In some embodiments, the PT is an NphB prenyltransferase. See, e.g., U.S. Pat. No. 7,544,498; and Kumano et al., Boorg Med Chem. 2008 Sep. 1; 16(17): 8117-8126, which are incorporated by reference in this application in their entireties. In some embodiments, a PT corresponds to NphB from Streptomyces sp. (see, e.g., UniprotKB Accession No. Q4R2T2; see also SEQ ID NO: 2 of U.S. Pat. No. 7,361,483). The protein sequence corresponding to UniprotKB Accession No. Q4R2T2 is provided by SEQ ID NO: 8:

(SEQ ID NO: 8)
MSEAADVERVYAAMEEAAGLLGVACARDKIYPLLSTFQDTLVEGGSVVVF
SMASGRHSTELDFSISVPTSHGDPYATVVEKGLFPATGHPVDDLLADTQK
HLPVSMFAIDGEVTGGFKKTYAFFPTDNMPGVAELSAIPSMPPAVAENAE
LFARYGLDKVQMTSMDYKKRQVNLYFSELSAQTLEAESVLALVRELGLHV
PNELGLKFCKRSFSVYPTLNWETGKIDRLCFAVISNDPTLVPSSDEGDIE
KFHNYATKAPYAYVGEKRTLVYGLTLSPKEEYYKLGAYYHITDVQRGLLK
AFDSLED.

A non-limiting example of a nucleic acid sequence encoding NphB is:

(SEQ ID NO: 9)
atgtcagaagccgcagatgtcgaaagagtttacgccgctatggaagaagc
cgccggtttgttaggtgttgcctgtgccagagataagatctacccattgt
tgtctacttttcaagatacattagttgaaggtggttcagttgttgttttc
tctatggcttcaggtagacattctacagaattggatttctctatctcagt
tccaacatcacatggtgatccatacgctactgttgttgaaaaaggtttat
ttccagcaacaggtcatccagttgatgatttgttggctgatactcaaaag
catttgccagtttctatgtttgcaattgatggtgaagttactggtggttt
caagaaaacttacgctttctttccaactgataacatgccaggtgttgcag
aattatctgctattccatcaatgccaccagctgttgcagaaaatgcagaa
ttatttgctagatacggtttggataaggttcaaatgacatctatggatta
caagaaaagacaagttaatttgtacttttctgaattatcagcacaaactt
tggaagctgaatcagttttggcattagttagagaattgggtttacatgtt
ccaaacgaattgggtttgaagttttgtaaaagatctttctcagtttatcc
aactttaaactgggaaacaggcaagatcgatagattatgtttcgcagtta
tctctaacgatccaacattggttccatcttcagatgaaggtgatatcgaa
aagtttcataactacgctactaaagcaccatatgcttacgttggtgaaaa
gagaacattagtttatggtttgactttatcaccaaaggaagaatactaca
agttgggtgcttactaccacattaccgacgtacaaagaggtttattgaaa
gcattcgatagtttagaagactaa.

In other embodiments, a PT corresponds to CsPT1, which is disclosed as SEQ ID NO:2 in U.S. Pat. No. 8,884,100 (C. sativa; corresponding to SEQ ID NO: 10 in this application):

(SEQ ID NO: 10)
MGLSSVCTFSFQTNYHTLLNPHNNNPKTSLLCYRHPKTPIKYSYNNFPSK
HCSTKSFHLQNKCSESLSIAKNSIRAATTNQTEPPESDNHSVATKILNFG
KACWKLQRPYTHIAFTSCACGLFGKELLHNTNLISWSLMFKAFFFLVAIL
CIASFTTTINQIYDLHIDRINKPDLPLASGEISVNTAWIMSIIVALFGLI
ITIKMKGGPLYIFGYCFGIFGGIVYSVPPFRWKQNPSTAFLLNFLAHIIT
NFTFYYASRAALGLPFELRPSFTFLLAFMKSMGSALALIKDASDVEGDTK
FGISTLASKYGSRNLTLFCSGIVLLSYVAAILAGIIWPQAFNSNVMLLSH
AILAFWLILQTRDFALTNYDPEAGRRFYEFMWKLYYAEYLVYVFI.

In some embodiments, a PT corresponds to CsPT4, which is disclosed as SEQ ID NO:1 in PCT Publication No. WO2019/071000, corresponding to SEQ ID NO: 11 in this application:

(SEQ ID NO: 11)
MGLSLVCTFSFQTNYHTLLNPHNKNPKNSLLSYQHPKTPIIKSSYDNFPS
KYCLTKNFHLLGLNSHNRISSQSRSIRAGSDQIEGSPHHESDNSIATKIL
NFGHTCWKLQRPYVVKGMISIACGLFGRELFNNRHLFSWGLMWKAFFALV
PILSFNFFAAIMNQIYDVDIDRINKPDLPLVSGEMSIETAWILSIIVALT
GLIVTIKLKSAPLFVFIYIFGIFAGFAYSVPPIRWKQYPFTNFLITISSH
VGLAFTSYSATTSALGLPFVWRPAFSFIIAFMTVMGMTIAFAKDISDIEG
DAKYGVSTVATKLGARNMTFVVSGVLLLNYLVSISIGIIWPQVFKSNIMI
LSHAILAFCLIFQTRELALANYASAPSRQFFEFIWLLYYAEYFVYVFI.

In some embodiments, a PT corresponds to a truncated CsPT4, which is provided as SEQ ID NO: 12:

MSAGSDQIEGSPHHESDNSIATKILNFGHTCWKLQRPYVVKGMISIACGL
FGRELFNNRHLFSWGLMWKAFFALVPILSFNFFAAIMNQIYDVDIDRINK
PDLPLVSGEMSIETAWILSIIVALTGLIVTIKLKSAPLFVFIYIFGIFAG
FAYSVPPIRWKQYPFTNFLITISSHVGLAFTSYSATTSALGLPFVWRPAF
SFIIAFMTVMGMTIAFAKDISDIEGDAKYGVSTVATKLGARNMTFVVSGV
LLLNYLVSISIGIIWPQVFKSNIMILSHAILAFCLIFQTRELALANYASA
PSRQFFEFIWLLYYAEYFVYVFI.

Functional expression of paralog C. sativa CBGAS enzymes in S. cerevisiae and production of the major cannabinoid CBGA has been reported (U.S. Patent Publication No. 2012/0144523, and Luo et al., Nature 2019 March; 567(7746):123-126). Luo et al. reported the production of CBGA in S. cerevisiae by expressing a truncated version of a C. sativa CBGAS, CsPT4, with its native signal peptide removed. Without being bound by a particular theory, the integral-membrane nature of C. sativa CBGAS enzymes may render functional expression of C. sativa CBGAS enzymes in heterologous hosts challenging. Removal of transmembrane domain(s) or signal sequences or use of prenyltransferases that are not associated with the membrane and are not integral membrane proteins may facilitate increased interaction between the enzyme and available substrate, for example in the cellular cytosol and/or in organelles that may be targeted using peptides that confer localization.

In some embodiments, the PT is a soluble PT. In some embodiments, the PT is a cytosolic PT. In some embodiments, the PT is a secreted protein. In some embodiments, the PT is not a membrane-associated protein. In some embodiments, the PT is not an integral membrane protein. In some embodiments, the PT does not comprise a transmembrane domain or a predicted transmembrane. In some embodiments, the PT may be primarily detected in the cytosol (e.g., detected in the cytosol to a greater extent than detected associated with the cell membrane). In some embodiments, the PT is a protein from which one or more transmembrane domains have been removed and/or mutated (e.g., by truncation, deletions, substitutions, insertions, and/or additions) so that the PT localizes or is predicted to localize in the cytosol of the host cell, or to cytosolic organelles within the host cell, or, in the case of bacterial hosts, in the periplasm. In some embodiments, the PT is a protein from which one or more transmembrane domains have been removed or mutated (e.g., by truncation, deletions, substitutions, insertions, and/or additions) so that the PT has increased localization to the cytosol, organelles, or periplasm of the host cell, as compared to membrane localization.

Within the scope of the term “transmembrane domains” are predicted or putative transmembrane domains in addition to transmembrane domains that have been empirically determined. In general, transmembrane domains are characterized by a region of hydrophobicity that facilitates integration into the cell membrane. Methods of predicting whether a protein is a membrane protein or a membrane-associated protein are known in the art and may include, for example amino acid sequence analysis, hydropathy plots, and/or protein localization assays.

In some embodiments, the PT is a protein from which a signal sequence has been removed and/or mutated so that the PT is not directed to the cellular secretory pathway. In some embodiments, the PT is a protein from which a signal sequence has been removed and/or mutated so that the PT is localized to the cytosol or has increased localization to the cytosol (e.g., as compared to the secretory pathway).

In some embodiments, the PT is a secreted protein. In some embodiments, the PT contains a signal sequence.

In some embodiments, a PT is a fusion protein. For example, a PT may be fused to one or more genes in the metabolic pathway of a host cell. In certain embodiments, a PT may be fused to mutant forms of one or more genes in the metabolic pathway of a host cell.

In some embodiments, a PT described in this application transfers one or more prenyl groups to any of positions 1, 2, 3, 4, or 5 in a compound of Formula (6), shown below:

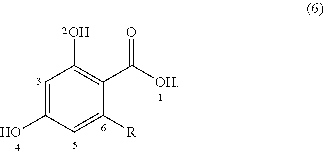

In some embodiments, the PT transfers a prenyl group to any of positions 1, 2, 3, 4, or 5 in a compound of Formula (6), shown below:

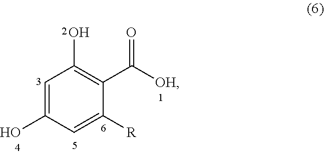

to form a compound of one or more of Formula (8w), Formula (8x), Formula (8′), Formula (8y), Formula (8z):

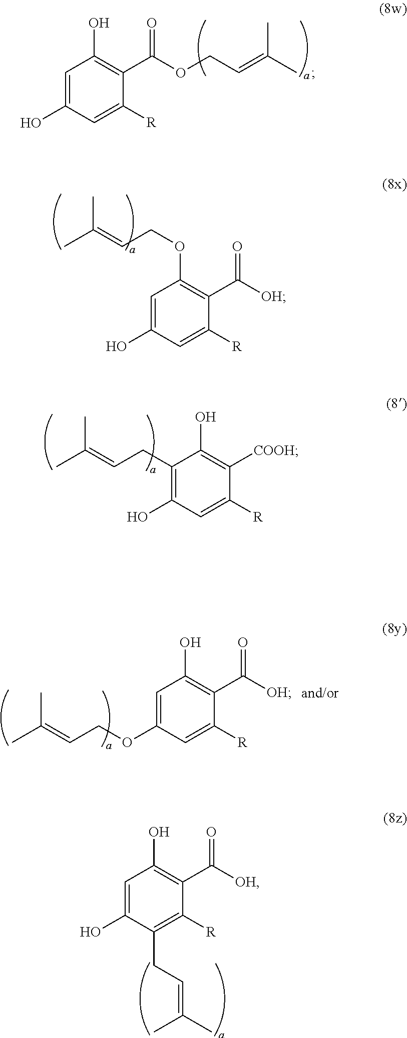

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

### Variants

Aspects of the disclosure relate to nucleic acids encoding any of the polypeptides (e.g., AAE, PKS, PKC, PT, or TS) described in this application. In some embodiments, a nucleic acid encompassed by the disclosure is a nucleic acid that hybridizes under high or medium stringency conditions to a nucleic acid encoding an AAE, PKS, PKC, PT, or TS and is biologically active. For example, high stringency conditions of 0.2 to 1×SSC at 65° C. followed by a wash at 0.2×SSC at 65° C. can be used. In some embodiments, a nucleic acid encompassed by the disclosure is a nucleic acid that hybridizes under low stringency conditions to a nucleic acid encoding an AAE, PKS, PKC, PT, or TS and is biologically active. For example, low stringency conditions of 6×SSC at room temperature followed by a wash at 2×SSC at room temperature can be used. Other hybridization conditions include 3×SSC at 40 or 50° C., followed by a wash in 1 or 2×SSC at 20, 30, 40, 50, 60, or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

Variants of enzyme sequences described in this application (e.g., AAE, PKS, PKC, PT, or TS, including nucleic acid or amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence (e.g., AAE, PKS, PKC, PT, or TS sequence). In some embodiments, sequence identity is determined over a region (e.g., a stretch of amino acids or nucleic acids, e.g., the sequence spanning an active site) of a sequence (e.g., AAE, PKS, PKC, PT, or TS sequence). For example, in some embodiments, sequence identity is determined over a region corresponding to at least 30%, at least 40%, at least 50%, at least 60%, at least 70%, at least 80%, at least 90%, at least 95%, or over 1⁰⁰% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST* protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul el al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers el al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

As used in this application, variant sequences may be homologous sequences. As used in this application, homologous sequences are sequences (e.g., nucleic acid or amino acid sequences) that share a certain percent identity (e.g., at least 5%, at least 100%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., AAE, PKS, PKC, PT, or TS enzyme variant) comprises a domain that shares a secondary structure (e.g., alpha helix, beta sheet) with a reference polypeptide (e.g., a reference AAE, PKS, PKC, PT, or TS enzyme). In some embodiments, a polypeptide variant (e.g., AAE, PKS, PKC, PT, or TS enzyme variant) shares a tertiary structure with a reference polypeptide (e.g., a reference AAE, PKS, PKC, PT, or TS enzyme). As a non-limiting example, a polypeptide variant (e.g., AAE, PKS, PKC, PT, or TS enzyme) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of the recombinant AAE, PKS, PKC, PT, or TS enzyme disclosed in this application are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation (e.g., substitution, deletion, and/or insertion) without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11; 10(9):2997-3011. The likelihood of observing a particular residue at a given position can be calculated. Without being bound by a particular theory, positions in sequences with high variability may be amenable to mutation (e.g., substitution, deletion, and/or insertion; e.g., PSSM score≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score≥0), can then be analyzed using the Rosetta energy function to determine the potential impact of the mutation on protein stability. Without being bound by a particular theory, potentially stabilizing amino acid mutations are desirable for protein engineering (e.g., production of functional homologs). In some embodiments, a potentially stabilizing amino acid mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, a coding sequence comprises an amino acid mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference coding sequence. In some embodiments, the coding sequence comprises an amino acid mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more substitutions, insertions, or deletions in the coding sequence do not alter the amino acid sequence of the coding sequence relative to the amino acid sequence of a reference polypeptide.

In some embodiments, the one or more mutations in a coding sequence do alter the amino acid sequence of the corresponding polypeptide relative to the amino acid sequence of a reference polypeptide. In some embodiments, the one or more mutations alters the amino acid sequence of the polypeptide relative to the amino acid sequence of a reference polypeptide and alter (enhance or reduce) an activity of the polypeptide relative to the reference polypeptide.

The activity (e.g., specific activity) of any of the recombinant polypeptides described in this application (e.g., AAE, PKS, PKC, PT, or TS) may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 3). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 3.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 5
Conservative Amino Acid Substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., AAE, PKS, PKC, PT, or TS) variant having a desired property and/or activity can be made by alteration of the coding sequence of the polypeptide (e.g., AAE, PKS, PKC, PT, or TS). Similarly, conservative amino acid substitutions in the amino acid sequence of a polypeptide to produce functionally equivalent variants of the polypeptide typically are made by alteration of the coding sequence of the recombinant polypeptide (e.g., AAE, PKS, PKC, PT, or TS).

Mutations (e.g., substitutions, insertions, additions, or deletions) can be made in a nucleic acid sequence by a variety of methods known to one of ordinary skill in the art. For example, mutations (e.g., substitutions, insertions, additions, or deletions) can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by CRISPR, or by insertions, such as insertion of a tag (e.g., a HIS tag or a GFP tag). Mutations can include, for example, substitutions, insertions, additions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Expression of Nucleic Acids in Host Cells

Aspects of the present disclosure relate to recombinant enzymes, functional modifications and variants thereof, as well as their uses. For example, the methods described in this application may be used to produce cannabinoids and/or cannabinoid precursors. The methods may comprise using a host cell comprising an enzyme disclosed in this application, cell lysate, isolated enzymes, or any combination thereof. Methods comprising recombinant expression of genes encoding an enzyme disclosed in this application in a host cell are encompassed by the present disclosure. In vitro methods comprising reacting one or more cannabinoid precursors or cannabinoids in a reaction mixture with an enzyme disclosed in this application are also encompassed by the present disclosure. In some embodiments, the enzyme is a TS.

A nucleic acid encoding any of the recombinant polypeptides (e.g., AAE, PKS, PKC, PT, or TS enzyme) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., AAE, PKS, PKC, PT, or TS enzyme) described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is hereby incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector so that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleic acid sequence of a gene described in this application is recoded. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1, GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. This may be used, for example, to controllably induce the expression of an enzyme. In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, an amino acid, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF, TEF2, RPL18B, SSA1, TDH2, PYK1, TPI1, HXT3, HXT7, ACT1, ADH1, ADH2, ENO2, and SOD1.

Other inducible promoters or constitutive promoters, including synthetic promoters, that may be known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed cannabinoid biosynthetic methods and host cells are exemplified with S. cerevisiae, but are also applicable to other host cells, as would be understood by one of ordinary skill in the art.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass.).

Other suitable host cells of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host cell of the present disclosure is C. glutamicum.

Suitable host cells of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Komagataella phaffii, formerly known as Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium. Brevibacterium, Butyrivibrio, Buchnera, Campestris, Camplyobacter, Clostridium, Corynebacterium, Chromatium, Coprococcus, Escherichia. Enterococcus, Enterobacter, Erwinia, Fusobacterium, Faecalibacterium, Francisella, Flavohacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types. In some embodiments, the plant is of the Cannabis genus in the family Cannabaceae. In certain embodiments, the plant is of the species Cannabis sativa, Cannabis indica, or Cannabis ruderalis. In other embodiments, the plant is of the genus Nicotiana in the family Solanaceae. In certain embodiments, the plant is of the species Nicotiana rustica.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart. Reduction of gene expression and/or gene inactivation in a host cell may be achieved through any suitable method, including but not limited to, deletion of the gene, introduction of a point mutation into the gene, selective editing of the gene and/or truncation of the gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies.

### Culturing of Host Cells

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place that involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., cannabinoid or cannabinoid precursor) may display some differences from the substrate in terms of solubility, toxicity, cellular accumulation and secretion and in some embodiments can have different fermentation kinetics.

In some embodiments, the cells of the present disclosure are adapted to produce cannabinoids or cannabinoid precursors in vivo. In some embodiments, the cells are adapted to secrete one or more enzymes for cannabinoid synthesis (e.g., AAE, PKS, PKC, PT, or TS). In some embodiments, the cells of the present disclosure are lysed, and the remaining lysates are recovered for subsequent use. In such embodiments, the secreted or lysed enzyme can catalyze reactions for the production of a cannabinoid or precursor by bioconversion in an in vitro or ex vivo process. In some embodiments, any and all conversions described in this application can be conducted chemically or enzymatically, in vitro or in vivo.

In some embodiments, the host cells of the present disclosure are adapted to produce cannabinoids or cannabinoid precursors in vivo. In some embodiments, the host cells are adapted to secrete one or more cannabinoid pathway substrates, intermediates, and/or terminal products (e.g., olivetol, THCA, THC, CBDA, CBD, CBGA, CBGVA, THCVA, CBDVA, CBCVA, or CBCA). In some embodiments, the host cells of the present disclosure are lysed, and the lysate is recovered for subsequent use. In such embodiments, the secreted substrates, intermediates, and/or terminal products may be recovered from the culture media.

### Purification and Further Processing

In some embodiments, any of the methods described in this application may include isolation and/or purification of the cannabinoids and/or cannabinoid precursors produced (e.g., produced in a bioreactor). For example, the isolation and/or purification can involve one or more of cell lysis, centrifugation, extraction, column chromatography, distillation, crystallization, and lyophilization.

The methods described in this application encompass production of any cannabinoid or cannabinoid precursor known in the art. Cannabinoids or cannabinoid precursors produced by any of the recombinant cells disclosed in this application or any of the in vitro methods described in this application may be identified and extracted using any method known in the art. Mass spectrometry (e.g., LC-MS, GC-MS) is a non-limiting example of a method for identification and may be used to extract a compound of interest.

In some embodiments, any of the methods described in this application further comprise decarboxylation of a cannabinoid or cannabinoid precursor. As a non-limiting example, the acid form of a cannabinoid or cannabinoid precursor may be heated (e.g., at least 90° C.) to decarboxylate the cannabinoid or cannabinoid precursor. See, e.g., U.S. Pat. Nos. 10,159,908, 10,143,706, 9,908,832 and 7,344,736. See also, e.g., Wang et al., Cannabis Cannabinoid Res. 2016; 1(1): 262-271.

### Compositions, Kits, and Administration

The present disclosure provides compositions, including pharmaceutical compositions, comprising a cannabinoid or a cannabinoid precursor, or pharmaceutically acceptable salt thereof, produced by any of the methods described in this application, and optionally a pharmaceutically acceptable excipient.

In certain embodiments, a cannabinoid or cannabinoid precursor described in this application is provided in an effective amount in a composition, such as a pharmaceutical composition. In certain embodiments, the effective amount is a therapeutically effective amount. In certain embodiments, the effective amount is a prophylactically effective amount.

Compositions, such as pharmaceutical compositions, described in this application can be prepared by any method known in the art. In general, such preparatory methods include bringing a compound described in this application (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils) as disclosed in this application.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic or semi-synthetic oils include, but are not limited to, butyl stearate, medium chain triglycerides (such as caprylic triglyceride and capric triglyceride), cyclomethicone, diethyl sebacate, dimethicone 360, isopropyl myristate, mineral oil, octyldodecanol, oleyl alcohol, silicone oil, and mixtures thereof. In certain embodiments, exemplary synthetic oils comprise medium chain triglycerides (such as caprylic triglyceride and capric triglyceride).

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described in this application are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described in this application with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described in this application may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described in this application include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described in this application.

A pharmaceutical composition described in this application can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided in this application are typically formulated in dosage unit form for ease of administration and uniformity of dosage. It will be understood, however, that the total daily usage of the compositions described in this application will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided in this application can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, compounds or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described in this application. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described in this application includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described in this application. In certain embodiments, a dose described in this application includes independently between 1 mg and 3 mg, inclusive, of a compound described in this application. In certain embodiments, a dose described in this application includes independently between 3 mg and 10 mg, inclusive, of a compound described in this application. In certain embodiments, a dose described in this application includes independently between 10 mg and 30 mg, inclusive, of a compound described in this application. In certain embodiments, a dose described in this application includes independently between 30 mg and 100 mg, inclusive, of a compound described in this application.

Dose ranges as described in this application provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described in this application, can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described in this application including a compound described in this application and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described in this application in a single dose or administered separately in different doses. The particular combination to employ in a regimen will take into account compatibility of the compound described in this application with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In some embodiments, one or more of the compositions described in this application are administered to a subject. In certain embodiments, the subject is an animal. The animal may be of either sex and may be at any stage of development. In certain embodiments, the subject is a human. In other embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

Also encompassed by the disclosure are kits (e.g., pharmaceutical packs). The kits provided may comprise a composition, such as a pharmaceutical composition, or a compound described in this application and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described in this application. In some embodiments, the pharmaceutical composition or compound described in this application provided in the first container and the second container a combined to form one unit dosage form.

Thus, in one aspect, provided are kits including a first container comprising a compound or composition described in this application. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described in this application further includes instructions for using the kit. A kit described in this application may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. A kit described in this application may include one or more additional pharmaceutical agents described in this application as a separate composition.

In some embodiments, the compositions include consumer product, such as comestible, cosmetic, toiletry, potable, inhalable, and wellness products. Exemplary consumer products include salves, waxes, powdered concentrates, pastes, extracts, tinctures, powders, oils, capsules, skin patches, sublingual oral dose drops, mucous membrane oral spray doses, makeup, perfume, shampoos, cosmetic soaps, cosmetic creams, skin lotions, aromatic essential oils, massage oils, shaving preparations, oils for toiletry purposes, lip balm, cosmetic oils, facial washes, moisturizing creams, moisturizing body lotions, moisturizing face lotions, bath salts, bath gels, bath soaps in liquid form, shower gels, bath bombs, hair care preparations, shampoos, conditioner, chocolate bars, brownies, chocolates, cookies, crackers, cakes, cupcakes, puddings, honey, chocolate confections, frozen confections, fruit-based confectionery, sugar confectionery, gummy candies, dragées, pastries, cereal bars, chocolate, cereal based energy bars, candy, ice cream, tea-based beverages, coffee-based beverages, and herbal infusions.

The present invention is further illustrated by the following Examples, which in no way should be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. However, mention of any reference, article, publication, patent, patent publication, and patent application cited in this application is not, and should not be taken as an acknowledgment or any form of suggestion that they constitute valid prior art or form part of the common general knowledge in any country in the world.

### EXAMPLES

### Example 1: Subcellular Targeting of Terminal Synthases

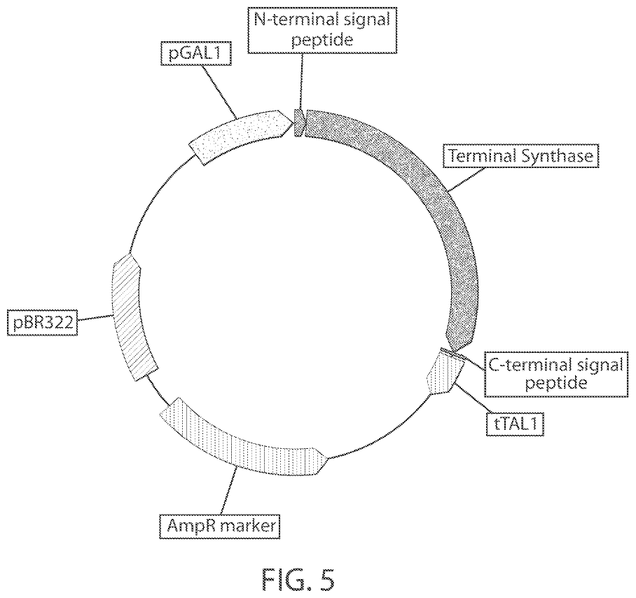

To identify intracellular locations within S. cerevisiae that would allow for proper folding and activity of expressed TS genes, a library of 29 strains expressing a C. sativa THCAS enzyme linked to a variety of N- and/or C-terminal signal peptides was designed to direct the THCAS enzymes to various subcellular compartments. Protein sequences were recoded in silico for expression in S. cerevisiae and synthesized in the integrative yeast expression vector shown in FIG. 5. Each enzyme expression construct was transformed into an S. cerevisiae CEN.PK strain that also expressed a prenyltransferase enzyme capable of catalyzing reaction R4 in FIG. 2. Information about library strains is provided in Table 7 and corresponding sequence information is provided in Table 20.

A tetrahydrocannabinolic acid (THCA) assay was conducted as follows: each thawed glycerol stock of THCAS transformants was stamped into a well of YEP+4% dextrose media. Samples were incubated at 30° C. and shaken in a shaking incubator for 2 days. A portion of each of the resulting cultures was stamped into a well of YEP+4% galactose+1 mM olivetolic acid (FIG. 1 Structure 6a). Samples were incubated at 20° C. and shaken in a shaking incubator for 4 days. Every 24 hours during those 4 days, 2% galactose and 1 mM olivetolic acid were spiked into the cultures. Sodium citrate buffer adjusted to pH 5.5 was added to each well at a final concentration of 100 mM. Samples were incubated at 20° C. and shaken in a shaking incubator for 2 days. A portion of each of the resulting production cultures was stamped into a well of phosphate buffered saline (PBS). Optical measurements were taken on a plate reader, with absorbance measured at 600 nm and fluorescence at 528 nm with 485 nm excitation. Samples were incubated at 30° C. and shaken in a shaking incubator for 2 days. 100% methanol was stamped into the production cultures in half-height deepwell plates. Plates were heat sealed and frozen. Samples were then thawed for 30 min and spun down at 4° C. A portion of the supernatant was stamped into half-area 96 well plates. THCA production in the samples was quantified in whole cell broth via LC-MS.

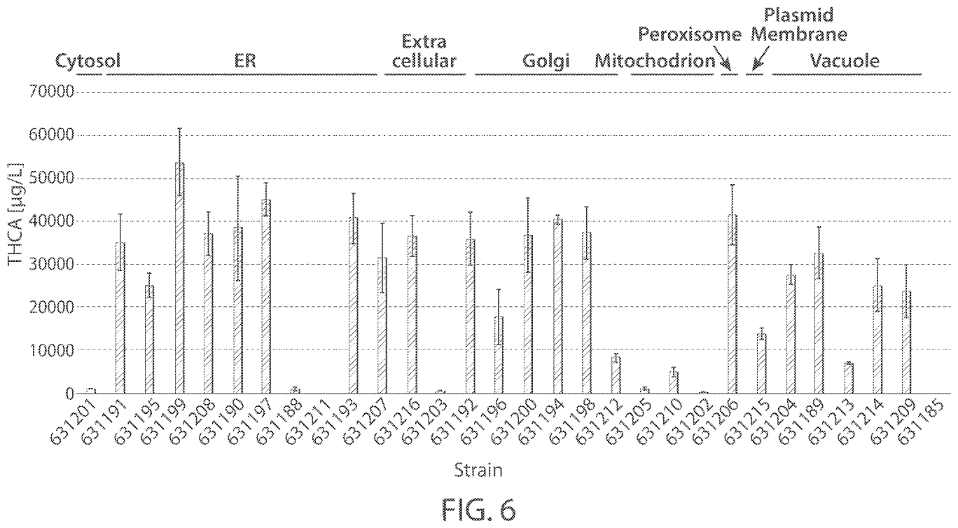

The library of THCAS expression constructs including N- and/or C-terminal signal peptides was assayed for activity in a screen using the assay described above. The THCAS expression constructs demonstrated measurable THCA production (FIG. 6). Strain IDs and their corresponding activities are shown in Table 6.

TABLE 6
THCA titers of THCAS expression constructs in S. cerevisiae

| | | | | Average | Standard |
|---|---|---|---|---|---|
| | Strain | N-terminal Signal Peptide/ | Predicted Target | THCA | Deviation |
| Strain | Type | C-terminal Sigal Peptide | Location | [μg/L] | THCA [μg/L] |
| 631185 | GFP | -/- | — | 0.00 | 0.00 |
| | negative | | | | |
| | control | | | | |
| 631188 | Library | -/HDEL | endoplasmic reticulum | 1153.70 | 271.06 |
| 631189 | Library | K. phaffii PEP4/- | Vacuole | 32647.87 | 5869.23 |
| 631190 | Library | Mfa2/HDEL | endoplasmic reticulum | 38533.71 | 11960.24 |
| 631191 | Library | YLR120C/HDEL | endoplasmic reticulum | 35162.01 | 6425.09 |
| 631192 | Library | YLR120C/KLD | Golgi | 35925.48 | 6164.50 |
| 631193 | Library | Mfa2/HDEL | endoplasmic reticulum | 40744.78 | 5687.94 |
| 631194 | Library | Mfa2/KLD | Golgi | 40394.58 | 1153.97 |
| 631195 | Library | Osm1p/HDEL | endoplasmic reticulum | 25031.34 | 3108.25 |
| 631196 | Library | Osm1p/KLD | Golgi | 17784.85 | 6412.46 |
| 631197 | Library | Sf leader/HDEL | endoplasmic reticulum | 45065.18 | 3937.30 |
| 631198 | Library | Sf leader/KLD | Golgi | 37305.08 | 6116.95 |
| 631199 | Library | Ost1_leader/HDEL | endoplasmic reticulum | 53925.15 | 7890.11 |
| 631200 | Library | Ost1_leader/KLD | Golgi | 36790.47 | 8661.71 |
| 631201 | Library | -/UBC6 | Cytosol | 1150.68 | 74.42 |
| 631202 | Library | -/PTS1 SKL | Peroxisome | 460.63 | 32.36 |
| 631203 | Library | -/CVIA from Mfa | Extracellular | 580.17 | 48.52 |
| 631204 | Library | YDR456W/- | Vacuole | 27670.08 | 2300.64 |
| 631205 | Library | YNL121C/- | Mitochondria | 1197.72 | 217.34 |
| 631206 | Library | YLR120C/- | plasma membrane | 41520.26 | 7025.15 |
| 631207 | Library | Mfa2/- | extracellular | 31574.55 | 8021.01 |
| 631208 | Library | Ost1 leader/- | endoplasmic reticulum | 37110.45 | 5192.99 |
| 631209 | Library | Sf leader/- | extracellular | 23732.83 | 6043.04 |
| 631210 | Library | OSM1-leader-T23L/- | mitochondria | 4951.01 | 1120.17 |
| 631211 | Library | ERG11-leader/- | endoplasmic reticulum | 0.00 | 0.00 |
| 631212 | Library | Osm1/- | mitochondria | 8341.30 | 920.72 |
| 631213 | Library | PRC1-11/- | vacuole | 6990.68 | 162.60 |
| 631214 | Library | PEP4 (short (1-24))/- | vacuole | 25111.88 | 6120.79 |
| 631215 | Library | PEP4 (long (2-76))/- | vacuole | 13885.96 | 1380.54 |
| 631216 | Library | Mfa2/- | extracellular | 36596.86 | 4670.46 |

Library strains comprising THCAS expression constructs with signal peptides that are expected to target the enzymes to organelles involved in the secretory pathway (e.g., the endoplasmic reticulum) were found to be critical for functional expression of the THCAS as measured by THCA production (FIG. 6 and Table 6). For example, strains t631199 and t631193 utilize the Ost1 leader sequence and the M4Falpha2 secretion tag, respectively, to target the THCAS to the secretory pathway. It is theorized that the THCAS harbored by both strains enters the endoplasmic reticulum as the starting point in the secretory pathway and is retained in that organelle via a HDEL endoplasmic reticulum retention tag. Without wishing to be bound by any theory, strains in which the signal peptides are expected to target the TSs to the secretory pathway may allow the TS to be exposed to subcellular environments beneficial for post-translational modifications (e.g., formation of a critical disulfide bridge in the oxidative environment of the endoplasmic reticulum and/or the addition of post-translational glycosylations in the endoplasmic reticulum and Golgi apparatus).

Strains in which the signal peptides are expected to target the TS to the plasma membrane also had functional expression. For example, strain t631206 harbored a THCAS N-terminally fused to the leader sequence of Ysp1 (UniProt Accession ID: P32329). The resulting enzyme is predicted to localize to the plasma membrane in a similar manner to Ysp1. Transport to the plasma membrane is mediated by the secretory machinery of S. cerevisiae, which should cause the THCAS protein to pass through the endoplasmic reticulum and/or the Golgi apparatus prior to being shuttled to the cell membrane. Routing through the endoplasmic reticulum and/or the Golgi apparatus may allow the TS to be post-translationally modified as discussed above.

Strains in which the signal peptides are expected to target the TS to vacuoles also had functional expression. For example, strain t631189 harbored a THCAS N-terminally fused to the signal peptide of Proteinase A from K. phaffii. The resulting enzyme is predicted to localize to the vacuole in a similar manner to Proteinase A. Transport to the vacuole is mediated by the secretory machinery of S. cerevisiae, which should cause the THCAS protein to pass through the endoplasmic reticulum and/or the Golgi apparatus. Routing through the endoplasmic reticulum and/or the Golgi apparatus may allow the TS to be post-translationally modified as discussed above.

It was also noted that both targeting to the cytosol and targeting exclusively to the endoplasmic reticulum without routing through the secretory pathway appeared to inhibit functional expression of a TS. For example, strain t631201 harbored a THCAS C-terminally fused to the signal peptide of UBC6 (UniProt Accession ID: P33296). This THCAS was found to have less activity than strains in which the THCAS was routed through the secretory pathway on the way to the endoplasmic reticulum. The resulting enzyme in strain t631201 is predicted to localize to the cytosolic side of the ER membrane in a similar manner to UBC6. Without wishing to be bound by a particular theory, the reduced activity of a TS localized to the cytosol may be caused by multiple factors including: the reductive environment of the cytosol precluding the formation of essential internal disulfide bridges of a TS and/or the lack of essential post-translation glycosylation of nascent peptides occurring in the cytosol.

The results of this assay suggest that the subcellular localization of a TS is a critical determinant of its activity in S. cerevisiae. Subcellular localizations which route a TS through the secretory machinery, specifically the endoplasmic reticulum and/or the Golgi apparatus, were found to be effective in ensuring that the TS is active in vivo. Multiple localization tags were found to accomplish this as shown in FIG. 6. In particular, an N-terminal signal peptide sourced from Ost1 or MFalpha2 combined with a C-terminal HDEL signal peptide produced the highest apparent THCAS activity out of the signal peptides tested.

TABLE 7
Strain Information associated with Example 1

| Strain | | |
|---|---|---|
| ID | N-terminal signal peptide information | C-terminal signal peptide information |
| 631185 | n/a | n/a |
| 631188 | n/a | Saccharomyces cerevisiae Protein disulfide- |
| | | isomerase protein endoplasmic reticulum |
| | | retention signal (HDEL from PDI1) (SEQ ID |
| | | NO: 17). |
| 631189 | Komagataella phaffii Vacuolar aspartyl protease | |
| | Proteinase A signal peptide (PEP4) (SEQ ID NO: | |
| | 706) | |
| 631190 | Saccharomyces cerevisiae Mating factor alpha | Saccharomyces cerevisiae Protein disulfide- |
| | protein leader peptide (mfalpha2_leader) (SEQ ID | isomerase protein endoplasmic reticulum |
| | NO: 16). | retention signal (HDEL from PDI1) (SEQ ID |
| | | NO: 17). |
| 631191 | Saccharomyces cerevisiae Aspartic proteinase 3 | Saccharomyces cerevisiae Protein disulfide- |
| | protein signal peptide (YLR120C_SP_native) | isomerase protein endoplasmic reticulum |
| | (SEQ ID NO: 608). | retention signal (HDEL from PDI1) (SEQ ID |
| | | NO: 17). |
| 631192 | Saccharomyces cerevisiae Aspartic proteinase 3 | Saccharomyces cerevisiae Golgi retention |
| | protein signal peptide (YLR120C_SP_native) | signal (KLD) (SEQ ID NO: 630). |
| | (SEQ ID NO: 608). | |
| 631193 | Saccharomyces cerevisiae Mating factor alpha | Saccharomyces cerevisiae Protein disulfide- |
| | protein leader peptide (mfalpha2_leader) (SEQ ID | isomerase protein endoplasmic reticulum |
| | NO: 16). | retention signal (HDEL from PDI1) (SEQ ID |
| | | NO: 17). |
| 631194 | Saccharomyces cerevisiae Mating factor alpha | Saccharomyces cerevisiae Golgi retention |
| | protein leader peptide (mfalpha2_leader) (SEQ ID | signal (KLD) (SEQ ID NO: 630). |
| | NO: 16). | |
| 631195 | Saccharomyces cerevisiae Fumarate reductase | Saccharomyces cerevisiae Protein disulfide- |
| | protein leader peptide (Osm1p leader, native) | isomerase protein endoplasmic reticulum |
| | (SEQ ID NO: 610). | retention signal (HDEL from PDI1) (SEQ ID |
| | | NO: 17). |
| 631196 | Saccharomyces cerevisiae Fumarate reductase | Saccharomyces cerevisiae Golgi retention |
| | protein leader peptide (Osm1p leader, native) | signal (KLD) (SEQ ID NO: 630). |
| | (SEQ ID NO: 610). | |
| 631197 | Saccharomycopsis fibuligera Glucoamylase | Saccharomyces cerevisiae Protein disulfide- |
| | protein leader peptide (Sf leader) (SEQ ID NO: | isomerase protein endoplasmic reticulum |
| | 612). | retention signal (HDEL from PDI1) (SEQ ID |
| | | NO: 17). |
| 631198 | Saccharomycopsis fibuligera Glucoamylase | Saccharomyces cerevisiae Golgi retention |
| | protein leader peptide (Sf leader) (SEQ ID NO: | signal (KLD) (SEQ ID NO: 630). |
| | 612). | |
| 631199 | Saccharomyces cerevisiae Dolichyl- | Saccharomyces cerevisiae Protein disulfide- |
| | diphosphooligosaccharide-protein | isomerase protein endoplasmic reticulum |
| | glycosyltransferase subunit 1 protein leader | retention signal (HDEL from PDI1) (SEQ ID |
| | peptide (Ost1 leader) (SEQ ID NO: 614). | NO: 17). |
| 631200 | Saccharomyces cerevisiae Dolichyl- | Saccharomyces cerevisiae Golgi retention |
| | diphosphooligosaccharide-protein | signal (KLD) (SEQ ID NO: 630). |
| | glycosyltransferase subunit 1 protein leader | |
| | peptide (Ost1 leader) (SEQ ID NO: 614). | |
| 631201 | n/a | Saccharomyces cerevisiae Ubiquitin- |
| | | conjugating enzyme E2 6 protein tail anchor |
| | | (tail anchor UBC6) (SEQ ID NO: 632). |
| 631202 | n/a | Saccharomyces cerevisiae peroxisomal citrate |
| | | synthase protein signal peptide (SKL from |
| | | CIT2) (SEQ ID NO: 634). |
| 631203 | n/a | Saccharomyces cerevisiae Mating hormone |
| | | A-factor 1 protein c-terminal prenylation |
| | | signal (CVIA from MFA1) (SEQ ID NO: |
| | | 636). |
| 631204 | Saccharomyces kudriavzevii Sodium/hydrogen | n/a |
| | exchanger protein signal peptide | |
| | (YDR456W_SP_native) (SEQ ID NO: 616). | |
| 631205 | Saccharomyces cerevisiae Mitochondrial import | n/a |
| | receptor subunit TOM70 signal peptide | |
| | (YNL121C_SP_native) (SEQ ID NO: 618). | |
| 631206 | Saccharomyces cerevisiae Aspartic proteinase 3 | n/a |
| | protein signal peptide (YLR120C_SP_native) | |
| | (SEQ ID NO: 608). | |
| 631207 | Saccharomyces cerevisiae Mating factor alpha | n/a |
| | signal peptide (mfalpha2_leader) (SEQ ID NO: | |
| | 16). | |
| 631208 | Saccharomyces cerevisiae Dolichyl- | n/a |
| | diphosphooligosaccharide-protein | |
| | glycosyltransferase subunit 1 protein leader | |
| | peptide (Ost1 leader) (SEQ ID NO: 614). | |
| 631209 | Saccharomycopsis fibuligera Glucoamylase | n/a |
| | protein leader peptide (Sf leader) (SEQ ID NO: | |
| | 612) | |
| 631210 | Saccharomyces cerevisiae Fumarate reductase | n/a |
| | protein leader peptide (OSM1-leader-T23L,S25L) | |
| | (SEQ ID NO: 620). | |
| 631211 | Saccharomyces cerevisiae Lanosterol 14-alpha | n/a |
| | demethylase protein leader peptide (ERG11- | |
| | leader) (SEQ ID NO: 622). | |
| 631212 | Saccharomyces cerevisiae Fumarate reductase | n/a |
| | protein leader peptide (Osm1p leader, native) | |
| | (SEQ ID NO: 610). | |
| 631213 | Saccharomyces cerevisiae Carboxypeptidase Y | n/a |
| | protein leader peptide (PRC1 1-111) (SEQ ID | |
| | NO: 624). | |
| 631214 | Saccharomyces cerevisiae Saccharopepsin protein | n/a |
| | vacuole targeting peptide (PEP4 1-24) (SEQ ID | |
| | NO: 626). | |
| 631215 | Saccharomyces cerevisiae Saccharopepsin protein | n/a |
| | vacuole targeting peptide (PEP4 2-76) (SEQ ID | |
| | NO: 628). | |
| 631216 | Saccharomyces cerevisiae Mating factor alpha | n/a |
| | protein leader peptide (mfalpha2_leader) (SEQ ID | |
| | NO: 16). | |

### Example 2: Screen to Identify Functional Expression of Tetrahydrocannabinolic Acid Synthases (THCASs)

To identify THCAS genes that can be functionally expressed in host cells, a library of 34 THCAS candidate genes was designed from sequences in C. sativa transcriptomic datasets. The THCAS candidate genes were recoded in silico for expression in S. cerevisiae and synthesized in the integrative yeast expression vector shown in FIG. 5. Each candidate enzyme expression construct was transformed into an S. cerevisiae CEN.PK strain that also expressed a prenyltransferase enzyme capable of catalyzing reaction R4 in FIG. 2. Strain 616313, expressing GFP, was included in the library screen as a negative control for enzyme activity. All candidate enzymes in the library were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

A terminal product assay was conducted as follows: each thawed glycerol stock of THCAS transformants was stamped into a well of YEP+4% dextrose media. Samples were incubated at 30° C. in a shaking incubator for 2 days. A portion of each of the resulting cultures was stamped into a well of YEP+4% galactose+1 mM olivetolic acid (FIG. 1, Structure 6a). Samples were incubated at 20° C. and shaken in a shaking incubator for 4 days. Every 24 hours during those 4 days, 2% galactose and 1 mM olivetolic acid were spiked into the cultures. Sodium citrate buffer adjusted to pH 5.5 was added to each well at a final concentration of 100 mM. Samples were incubated at 20° C. in a shaking incubator for 2 days. A portion of each of the resulting production cultures was stamped into a well of phosphate buffered saline (PBS). Optical measurements were taken on a plate reader, with absorbance measured at 600 nm and fluorescence at 528 nm with 485 nm excitation. Samples were incubated at 30° C. in a shaking incubator for 2 days. 100% methanol was stamped into the production cultures in half-height deepwell plates. Plates were heat sealed and frozen. Samples were then thawed for 30 min and spun down at 4° C. A portion of the supernatant was stamped into half-area 96 well plates. THCA, CBDA, and CBCA production in the samples was quantified via Liquid chromatography-mass spectrometry (LC-MS).

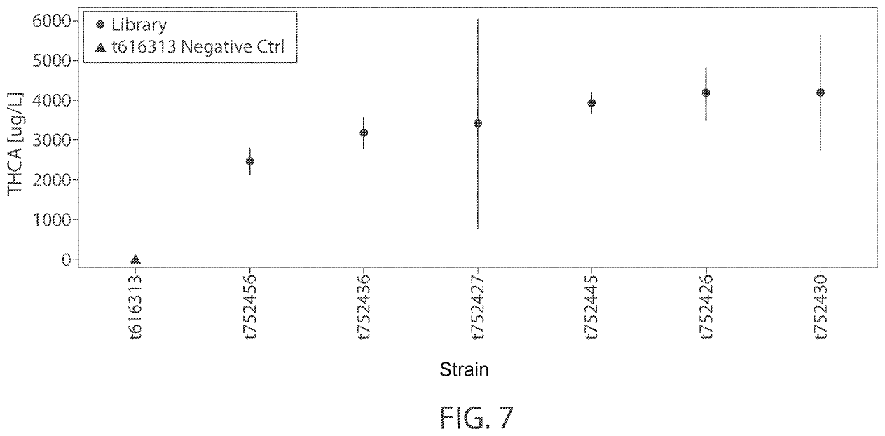

The library of candidate THCAS enzymes was assayed for activity in a screen using the assay described above. LC-MS analysis revealed 6 candidate THCASs that demonstrated measurable amounts of THCA (FIG. 7). The data represent the average of four biological replicates. Strain IDs and their corresponding activity are shown in Table 8. For example, as shown in Table 8, strain 752426 produced 4181.53 μg/L with a standard deviation of 750.62 μg/L.

TABLE 8
THCA titers of THCAS candidate enzymes in S. cerevisiae

| | THCAS protein SEQ ID | | | |
|---|---|---|---|---|
| | NO (without signal | | | Standard |
| Strain | peptides or N-terminal | | Mean | Deviation |
| ID | methionine) | Strain Type | THCA [μg/L] | THCA [μg/L] |
| 616313 | — | GFP Negative Ctrl | 0 | 0 |
| 752426 | 37 | Library | 4181.53 | 750.62 |
| 752427 | 43 | Library | 3416.75 | 3017.77 |
| 752430 | 40 | Library | 4194.83 | 1674.79 |
| 752436 | 39 | Library | 3179.48 | 419.78 |
| 752445 | 38 | Library | 3931.0 | 314.04 |
| 752456 | 42 | Library | 2469.28 | 346.57 |

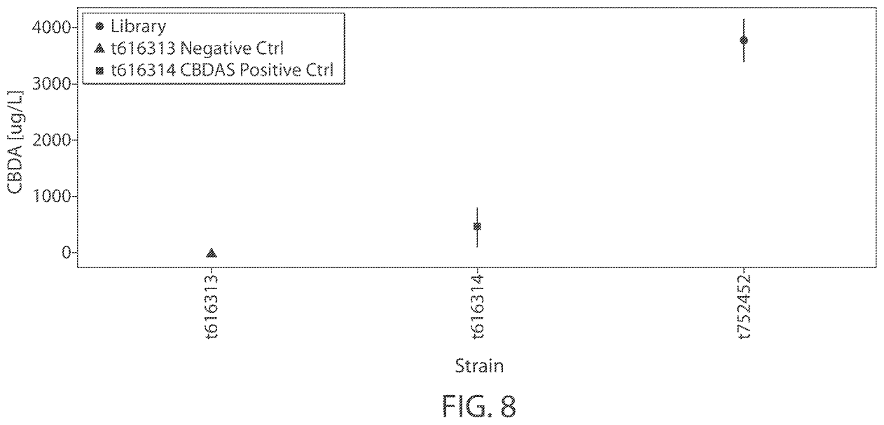

Since product promiscuity has been noted among the Cannabis terminal synthases, the production of cannabidiolic acid (CBDA) was quantified via LC-MS to determine whether library members in the terminal product assay also display cannabidiolic acid synthase (CBDAS) activity. Strain t616314, expressing a CBDAS from C. sativa set forth as SEQ ID NO: 136, was included in the library as a positive control for CBDAS activity. 1 library member demonstrated detectable CBDA (FIG. 8). Strain IDs and their corresponding activity are shown in Table 9. The data represent the average of four biological replicates. As shown in Table 9, strain 752452 produced 3765.4 μg/L CBDA with a standard deviation of 420.17 μg/L.

TABLE 9
CBDA titers of CBDAS candidate enzymes in S. cerevisiae

| | CBDAS protein SEQ ID | | | |
|---|---|---|---|---|
| | NO (without signal | | | Standard |
| Strain | peptides or N-terminal | | Mean | Deviation |
| ID | methionine) | Strain Type | CBDA [μg/L] | CBDA [μg/L] |
| 616313 | N/A | GFP Negative Control | 0 | 0 |
| 616314 | 136 | Positive Control (CBDAS) | 457.3 | 360.6 |
| 752452 | 36 | Library | 3765.4 | 420.17 |

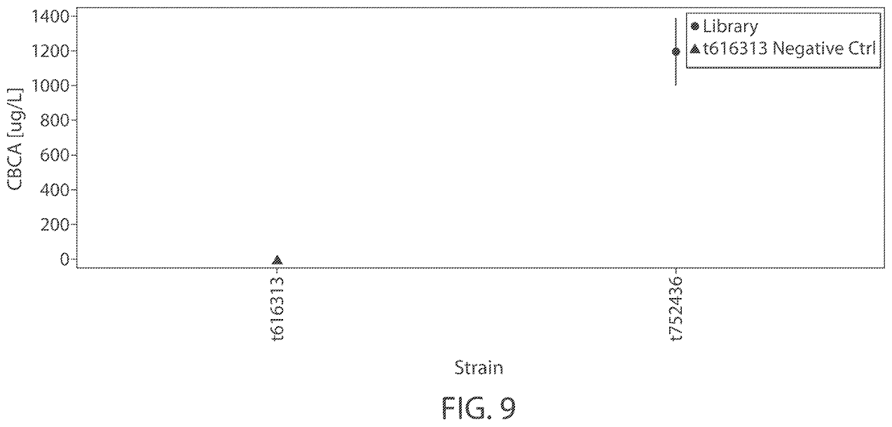

To determine whether library members also display cannabichromenic acid synthase (CBCAS) activity, the production of cannabichromenic acid (CBCA) was quantified via LC-MS. Enzyme candidates previously annotated as putative Cannabis CBCAS demonstrated no CBCAS activity. Surprisingly, 1 library member that demonstrated detectable THCA also demonstrated detectable CBCA (FIG. 9). The data represents the average of four biological replicates. Strain IDs and their corresponding activity are shown in Table 10. As shown in Table 10, strain 752436 produced 1198.58 μg/L CBCA with a standard deviation of 209.39 μg/L. Strain IDs and their corresponding sequences are shown in Table 21.

TABLE 10
CBCA titers of CBCAS candidate enzymes in S. cerevisiae

| | CBCAS protein SEQ ID | | | |
|---|---|---|---|---|
| | NO (without signal | | | Standard |
| | peptides or N-terminal | | Mean | Deviation |
| Strain | methionine) | Strain type | CBDA [μg/L] | CBDA [μg/L] |
| 616313 | N/A | GFP Negative Control | 0 | 0 |
| 752436 | 39 | Library | 1198.58 | 209.39 |

### Example 3: Additional Screen to Identify Functional Expression of Terminal Synthases

To identify additional terminal synthase genes that can be functionally expressed in host cells, a second library of 1380 candidate terminal synthase genes was designed. Candidate terminal synthases included individual point mutation variants and multiple point mutation variants of a C. sativa THCAS (e.g., Uniprot Accession: Q8GTB6) and a C. sativa CBDAS (e.g., Uniprot Accession: A6P6V9), terminal synthase candidates designed from sequences in C. sativa transcriptomic datasets, and “ancestral” terminal synthases inferred by probabilistic models applied to phylogenies of the terminal synthases and their homologs. Point mutations were designed based on proximity to the active site, PSSM/Rosetta energy calculations for improved stability and/or abundance, mutations of glycosylation sites, and/or ancestral reconstructions.

The terminal synthase candidate genes were recoded in silico for expression in S. cerevisiae. These sequences were synthesized in the integrative yeast expression vector shown in FIG. 5. Each candidate enzyme expression construct was transformed into an S. cerevisiae CEN.PK strain that also expressed a prenyltransferase enzyme capable of catalyzing reaction R4 in FIG. 2. Strain 616313, expressing GFP, was included in the library screen as a negative control for enzyme activity. Strain 701870, expressing a THCAS from C. sativa set forth as SEQ ID NO: 284, was included in the library as a positive control for THCAS activity and was used to establish hit ranking of candidate THCAS enzymes. Strain 616314, expressing a CBDAS from C. sativa set forth as SEQ ID NO: 136, was included in the library as a positive control for CBDAS activity and was also used to establish hit ranking for candidate CBDAS enzymes. A putative C. sativa CBCAS enzyme that was previously disclosed was not found to be active. Instead, a C. sativa THCAS enzyme (set forth in SEQ ID NO: 21) was found to demonstrate CBCAS activity in addition to THCAS activity using the assays described in this Example, and was accordingly used as a positive control for CBCAS activity (strain 616315).

All candidate enzymes in the library, as well as the enzymes expressed by positive control strains 701870, 616314, and 616315 were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

The terminal product assay was conducted in the same manner as described in Example 2. THCA, CBDA, and CBCA production in each sample were quantified via Liquid chromatography-mass spectrometry (LC-MS).

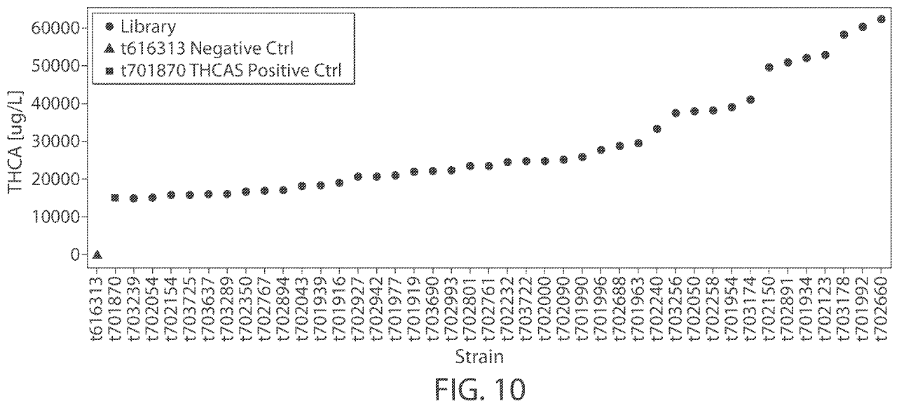

Of the 1380 candidate terminal synthases assayed, 41 produced more THCA than positive control strain 701870 (FIG. 10). The data represents the average of two biological replicates. Strain IDs and their corresponding activity are shown in Table 10. For example, as shown in Table 11, strain 701916 produced 18800.4 μg/L THCA. Strain 1Ds and their corresponding sequences are shown in Table 22.

TABLE 11
THCA titers of THCAS candidate enzymes in S. cerevisiae

| | THCA protein SEQ ID NO | | Mean |
|---|---|---|---|
| Strain | (without signal peptides or | | THCA |
| ID | N-terminal methionine) | Strain Type | [μg/L] |
| 616313 | N/A | GFP Negative Control | 10.6 |
| 701870 | 284 | Positive Control | 14706.2 |
| | | (C. sativa THCAS) | |
| 701916 | 138 | Library | 18800.4 |
| 701919 | 140 | Library | 21623.2 |
| 701934 | 141 | Library | 51917.7 |
| 701939 | 285 | Library | 18128.1 |
| 701954 | 286 | Library | 38816.3 |
| 701963 | 287 | Library | 29334.4 |
| 701977 | 288 | Library | 20745.7 |
| 701990 | 289 | Library | 25646.1 |
| 701992 | 144 | Library | 60171.5 |
| 701996 | 290 | Library | 27496.8 |
| 702000 | 291 | Library | 24592.9 |
| 702043 | 292 | Library | 17862.8 |
| 702050 | 293 | Library | 37749.3 |
| 702054 | 294 | Library | 14819.1 |
| 702090 | 295 | Library | 24898.8 |
| 702123 | 155 | Library | 52639.5 |
| 702150 | 158 | Library | 49485.4 |
| 702154 | 296 | Library | 15523.1 |
| 702232 | 297 | Library | 24238.7 |
| 702240 | 298 | Library | 33056.0 |
| 702258 | 164 | Library | 38012.6 |
| 702350 | 178 | Library | 16354.7 |
| 702660 | 198 | Library | 62259.9 |
| 702688 | 199 | Library | 28542.9 |
| 702761 | 299 | Library | 23306.7 |
| 702767 | 300 | Library | 16585.2 |
| 702801 | 301 | Library | 23258.5 |
| 702891 | 200 | Library | 50769.3 |
| 702894 | 302 | Library | 16799.2 |
| 702927 | 303 | Library | 20384.9 |
| 702942 | 304 | Library | 20404.8 |
| 702993 | 305 | Library | 22118.6 |
| 703174 | 306 | Library | 40926.8 |
| 703178 | 203 | Library | 58146.5 |
| 703239 | 307 | Library | 14713.8 |
| 703256 | 308 | Library | 37302.0 |
| 703289 | 309 | Library | 15747.5 |
| 703637 | 310 | Library | 15628.4 |
| 703690 | 311 | Library | 21803.7 |
| 703722 | 312 | Library | 24552.7 |
| 703725 | 313 | Library | 15524.9 |

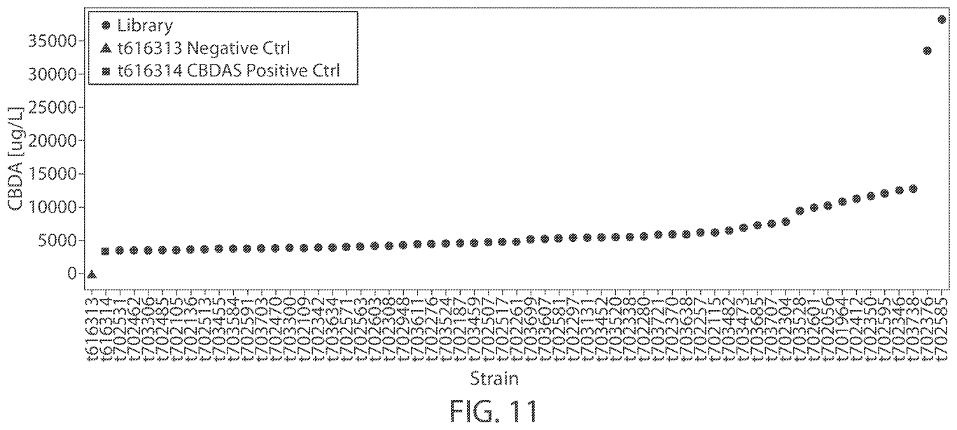

Additionally, 62 candidate terminal synthases assayed demonstrated mean CBDA titers greater than that of the positive control 616314 (FIG. 11). The data represents the average of two biological replicates. Strain IDs and their corresponding activity are shown in Table 12. For example, as shown in Table 12, strain 701964 produced 10674.6 pig/L CBDA. Strain Ds and their corresponding sequences are shown in Table 22. Positive control strain 616314 demonstrated considerably higher CBDA production in the screen conducted in Example 3 than in the screen conducted in Example 2. Such differences may be attributable to the high throughput nature of the screening assays and differences between the growth conditions used during the two screens. As would be understood by one of ordinary skill in the art, the relative activity for candidate terminal synthases in a given screen is determined relative to control strains tested within the same screen under the same growth conditions.

TABLE 12
CBDA titers of CBDAS candidate enzymes in S. cerevisiae

| | CBDA protein SEQ ID | | |
|---|---|---|---|
| | NO (without signal | | Mean |
| Strain | peptides or N-terminal | | CBDA |
| ID | methionine) | Strain Type | [μg/L] |
| 616313 | 135 | GFP Negative Control | 0 |
| 616314 | 136 | Positive Control | 3264.431 |
| | | (CBDAS) | |
| 701964 | 143 | Library | 10674.6 |
| 702056 | 149 | Library | 10038.3 |
| 702105 | 151 | Library | 3368.8 |
| 702109 | 152 | Library | 3735.5 |
| 702115 | 153 | Library | 6054.6 |
| 702136 | 156 | Library | 3486.9 |
| 702187 | 160 | Library | 4416.3 |
| 702257 | 163 | Library | 6009.7 |
| 702261 | 165 | Library | 4621.9 |
| 702276 | 166 | Library | 4323.2 |
| 702280 | 168 | Library | 5437.8 |
| 702297 | 170 | Library | 5264.8 |
| 702304 | 171 | Library | 7678.5 |
| 702308 | 172 | Library | 4048.8 |
| 702338 | 175 | Library | 5407.8 |
| 702342 | 176 | Library | 3770.6 |
| 702346 | 177 | Library | 12380.6 |
| 702350 | 178 | Library | 11520.1 |
| 702370 | 179 | Library | 5722.1 |
| 702376 | 180 | Library | 33308.5 |
| 702412 | 182 | Library | 11128.6 |
| 702462 | 183 | Library | 3335.8 |
| 702470 | 184 | Library | 3686.9 |
| 702485 | 185 | Library | 3360.4 |
| 702507 | 186 | Library | 4561.6 |
| 702513 | 187 | Library | 3504 |
| 702517 | 188 | Library | 4611 |
| 702531 | 189 | Library | 3320.5 |
| 702563 | 190 | Library | 3947.8 |
| 702571 | 191 | Library | 3888 |
| 702581 | 192 | Library | 5132.8 |
| 702585 | 193 | Library | 37977 |
| 702591 | 194 | Library | 3640.5 |
| 702595 | 195 | Library | 11914.1 |
| 702601 | 196 | Library | 9766.7 |
| 702603 | 197 | Library | 4000.2 |
| 702948 | 201 | Library | 4171.4 |
| 703131 | 202 | Library | 5298.7 |
| 703300 | 204 | Library | 3729.3 |
| 703306 | 205 | Library | 3341.2 |
| 703452 | 207 | Library | 5307.5 |
| 703455 | 208 | Library | 3638.3 |
| 703459 | 209 | Library | 4458.8 |
| 703473 | 210 | Library | 6768.2 |
| 703482 | 211 | Library | 6324.4 |
| 703520 | 212 | Library | 5350.2 |
| 703524 | 213 | Library | 4349.4 |
| 703528 | 214 | Library | 9277.4 |
| 703584 | 215 | Library | 3639.7 |
| 703607 | 216 | Library | 5067.5 |
| 703611 | 217 | Library | 4306.7 |
| 703634 | 218 | Library | 3801.2 |
| 703638 | 219 | Library | 5746.3 |
| 703685 | 220 | Library | 7115.2 |
| 703699 | 221 | Library | 5055.9 |
| 703703 | 222 | Library | 3676.3 |
| 703707 | 223 | Library | 7337.2 |
| 703721 | 224 | Library | 5717.1 |
| 703738 | 225 | Library | 12609.1 |

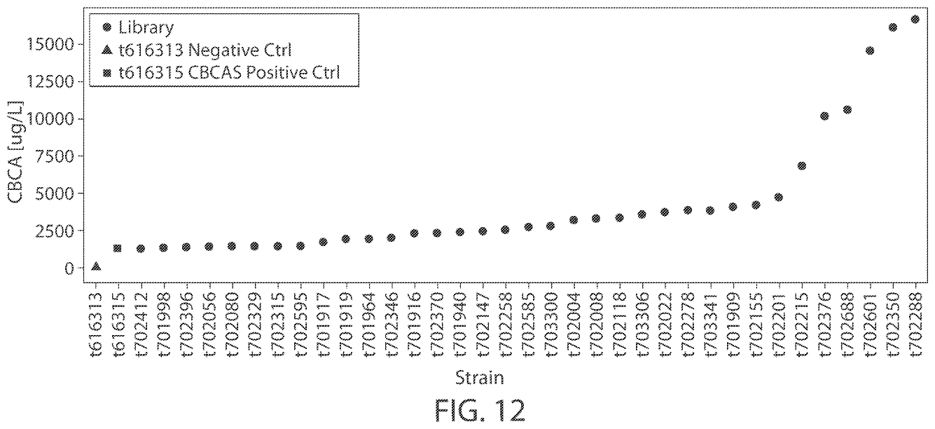

36 candidate terminal synthases assayed demonstrated mean CBCA titers greater than that of the positive control 616315 (FIG. 12). The data represents the average of two biological replicates. Strain IDs and their corresponding activity are shown in Table 13. For example, as shown in Table 13, strain 701909 produced 4064.4 μg/L CBCA. Strain IDs and their corresponding sequences are shown in Table 22.

TABLE 13
CBCA titers of CBCAS candidate enzymes in S. cerevisiae

| | CBCA protein SEQ ID | | |
|---|---|---|---|
| | NO (without signal | | Mean |
| Strain | peptides or N-terminal | | CBCA |
| ID | methionine) | Strain Type | [μg/L] |
| 616313 | — | GFP Negative Control | 82.7 |
| 616315 | 21 | Positive Control | 1233.9 |
| | | (C. sativa THCAS) | |
| 701909 | 137 | Library | 4064.4 |
| 701916 | 138 | Library | 2276.1 |
| 701917 | 139 | Library | 1692.4 |
| 701919 | 140 | Library | 1898.9 |
| 701940 | 142 | Library | 2363.1 |
| 701964 | 143 | Library | 1906.4 |
| 701998 | 145 | Library | 1308.4 |
| 702004 | 146 | Library | 3174.3 |
| 702008 | 147 | Library | 3290.3 |
| 702022 | 148 | Library | 3718.5 |
| 702056 | 149 | Library | 1390.1 |
| 702080 | 150 | Library | 1393.4 |
| 702118 | 154 | Library | 3349.2 |
| 702147 | 157 | Library | 2419.7 |
| 702155 | 159 | Library | 4183.0 |
| 702201 | 161 | Library | 4688.6 |
| 702215 | 162 | Library | 6804.2 |
| 702258 | 164 | Library | 2506.6 |
| 702278 | 167 | Library | 3813.0 |
| 702288 | 169 | Library | 16597.8 |
| 702315 | 173 | Library | 1414.6 |
| 702329 | 174 | Library | 1411.9 |
| 702346 | 177 | Library | 1990.6 |
| 702350 | 178 | Library | 16078.8 |
| 702370 | 179 | Library | 2292.1 |
| 702376 | 180 | Library | 10128.8 |
| 702396 | 181 | Library | 1358.4 |
| 702412 | 182 | Library | 1246.4 |
| 702585 | 193 | Library | 2694.3 |
| 702595 | 195 | Library | 1437.9 |
| 702601 | 196 | Library | 14507.0 |
| 702688 | 199 | Library | 10566.0 |
| 703300 | 204 | Library | 2790.4 |
| 703306 | 205 | Library | 3554.0 |
| 703341 | 206 | Library | 3815.8 |

A number of strains produced multiple TS products, demonstrating two or more of THCAS, CBDAS and CBCAS activity. In particular, the following strains demonstrated both THCAS and CBCAS activity: strain 701916 which expresses a TS (SEQ ID NO: 138) that includes amino acid substitutions R311Q, K40E, H41Y, V46P, L5I F, V52L, 163V, 174T, N90V, T96S, V103I, A116S, and P542L relative to SEQ ID NO: 14, strain 701919, which expresses a TS (SEQ ID NO: 140) that includes amino acid substitution V288L relative to SEQ ID NO: 14; strain 702258, which expresses a TS (SEQ ID NO: 164) that includes amino acid substitutions R31Q, K40Q, 1H41Y, 174T, N90V, V129I, V288L, K296R, F345L, F360Y, A411V, E424D, H494P, and A495E relative to SEQ ID NO: 14; and strain 702688, which expresses a TS (SEQ ID NO: 199) that includes amino acid substitutions I63L, L443I, T446I, V462I, S464N, L479M, H494F, A495E, N528D, P542L, and H543R relative to SEQ ID NO: 14.

The following strains demonstrated both CBDAS and CBCAS activity: strain 701964, which expresses a TS (SEQ ID NO: 143) that includes amino acid substitution H69Q relative to SEQ ID NO: 13, strain 702056, which expresses a TS (SEQ ID NO: 149) that includes amino acid substitution H69R relative to SEQ ID NO: 13; strain 702346, which expresses a TS (SEQ ID NO: 177) that includes amino acid substitution A414M relative to SEQ ID NO: 13; strain 702370, which expresses a TS (SEQ ID NO: 179) that includes amino acid substitution Y416F relative to SEQ ID NO: 13; strain 702376, which expresses a TS (SEQ ID NO: 180) that includes amino acid substitution S116A relative to SEQ ID NO: 13; strain 702412, which expresses a TS (SEQ ID NO: 182) that includes amino acid substitution S116G relative to SEQ ID NO: 13; strain 702585, which expresses a TS (SEQ ID NO: 193) that includes amino acid substitution A414V relative to SEQ ID NO: 13; strain 702595, which expresses a TS (SEQ ID NO: 195) that includes amino acid substitution S100A relative to SEQ ID NO: 13; strain 702601, which expresses a TS (SEQ ID NO: 196) that includes amino acid substitutions E40Q, P46A, A49S, P51A, F53L, I54V, H58N, Q60P, A97G, S98T, V131I, H138R, F171L, G175A, V183A, N239S, A244V, K249R, I259M, G270E, F275V, V290L, K298R, H304Q, V311I, G313S, H320L, E346Q, F347L, T353I, F362Y, N363D, A365T, K379Q, K380N, T381A, S384K, A398V, E426D, T448I, 1461L, V464I, S466N, T471M, T494N, E497K, A527V, P544R, and H546R relative to SEQ ID NO: 20; strain 703300, which expresses a TS (SEQ ID NO: 204) that includes amino acid substitution E441S relative to SEQ ID NO: 13; and strain 703306, which expresses a TS (SEQ ID NO: 205) that includes amino acid substitution E441T relative to SEQ ID NO: 13.

Strain 702350 demonstrated THCAS, CBDAS, and CBCAS activity. This strain expresses a TS (SEQ ID NO: 178) that includes amino acid substitutions R31Q, K40Q, H41Y, V46A, A47T, P49A, H56N, Q58P, I63V, I74T, N90V, A95G, V129I, H136R, G173A, V181A, N237S, A242V, K247R, 1257M, G268E, F273V, V288L, K296R, H302Q, V309I, G311S, H318L, E344Q, F345L, T351I, F360Y, N361D, A363T, K377Q, K378N, T379A, S382K, A396V, A411V, E424D, T446I, I459L, V462I, S464N, T469M, T492N, H494P, A495K, P542R, and H544R relative to SEQ ID NO: 14.

### Example 4: Screen to Identify Functional Expression of Additional Terminal Synthases

To identify additional terminal synthase genes that can be functionally expressed in host cells, a library of approximately 1762 candidate terminal synthases was designed using ancestral sequence reconstruction and recombination of single-mutations identified in Example 3 that demonstrated improvements in terminal synthase activity.

Ancestral Sequence Reconstruction: Terminal synthase candidates sourced from publicly available RNAseq datasets were used to generate multiple protein phylogenies. Putative “ancestral” terminal synthases were constructed at the nodes of these phylogenetic trees via a phylogenetic analysis of maximum likelihood.

Recombination of single mutations: Terminal synthase candidates in Example 3 included single point mutants of two C. sativa THCASs (Uniprot Accession: I1V0C5 and Q8GTB6) and one C. sativa CBDAS (Uniprot Accession: A6P6V9). A Multiple Sequence Alignment (MSA) of these mutant sequences and other terminal synthase homologs was generated and used as the basis for learning interacting positions within terminal synthase candidates. This was used to inform the mutation space to explore and subsequently recombine into the aforementioned templates.

The terminal synthase candidate genes were recoded in silico for expression in S. cerevisiae and synthesized in the integrative yeast expression vector shown in FIG. 5. Each candidate enzyme expression construct was transformed into an S. cerevisiae CEN.PK strain that also expressed a prenyltransferase enzyme capable of catalyzing reaction R4 in FIG. 2. Strain 616313, expressing GFP, was included in the library screen as a negative control for enzyme activity. All candidate enzymes in the library were expressed with an N-terminal MFalpha2 signal peptide (SEQ ID NO. 16) and a C-terminal HDEL signal peptide (SEQ ID NO: 17). A methionine residue was also added at the amino terminus of SEQ ID NO: 16.

Strains t807949 and t820182, expressing two different C. sativa THCASs (corresponding to Uniprot Accession: I1V0C5 and Q8GTB6, respectively), were included in the library as positive controls for THCAS activity. Strain t807973, expressing a C. sativa CBDAS (corresponding to Uniprot Accession: A6P6V9), was included in the library as a positive control for CBDAS activity. Positive control sequences were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16. Strain t807914, expressing a GFP fluorescent reporter was included in the library as a negative control.

A terminal product assay was conducted as follows: each thawed glycerol stock of terminal synthase transformants was stamped into a well of YEP+4% dextrose media. Samples were incubated at 30° C. in a shaking incubator for 2 days. A portion of each of the resulting cultures was stamped into a well of YEP+4% galactose+1 mM olivetolic acid (FIG. 1, Structure 6a). Samples were incubated at 20° C. and shaken in a shaking incubator for 4 days. Every 24 hours during those 4 days, 2% galactose and 1 mM olivetolic acid were spiked into the cultures. Sodium citrate buffer adjusted to pH 5.5 was added to each well at a final concentration of 100 mM. Samples were incubated at 20° C. in a shaking incubator for 2 days. A portion of each of the resulting production cultures was stamped into a well of PBS. Optical measurements were taken on a plate reader, with absorbance measured at 600 nm and fluorescence at 528 nm with 485 nm excitation. Samples were incubated at 30° C. in a shaking incubator for 2 days. 100% methanol was stamped into the production cultures in half-height deepwell plates. Plates were heat sealed and frozen. Samples were then thawed for 30 min and spun down at 4° C. A portion of the supernatant was stamped into half-area 96 well plates. THCA, CBDA, and CBCA production in the samples was quantified via LC-MS.

142 strains were elevated to a secondary assay to confirm their activity. The secondary assay was performed in the same manner as the primary assay with the following exceptions: four biological replicates were included for each strain, and a parallel assay was run wherein olivetolic acid was replaced with divaric acid. In addition to THCA, CBDA, and CBCA their counterparts derived from divaric acid THCVA, CBDVA, and CBCVA respectively were also quantified via LC/MS.

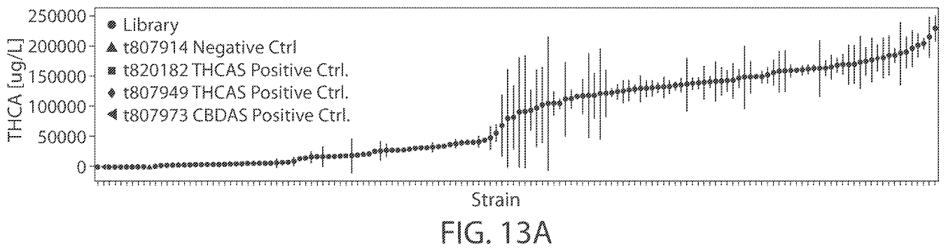

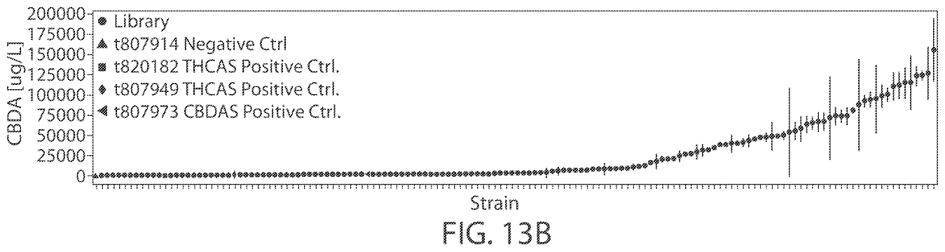

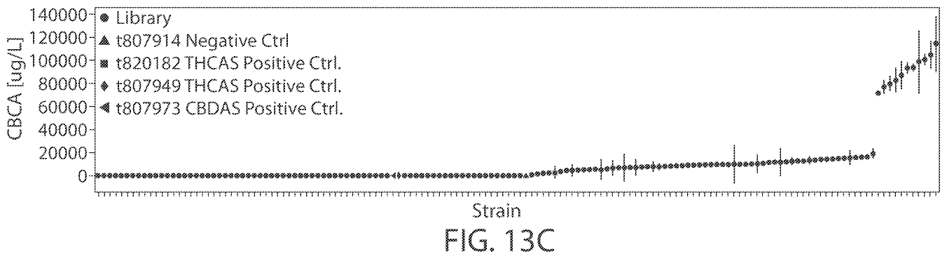

101 strains demonstrated mean THCA titers greater than the mean THCA titer of the t820182 positive control (FIG. 13A, Table 14). The t820182 positive control expresses a THCAS corresponding to the sequence associated with Uniprot Accession Q8GTB6, except that instead of its endogenous signal peptide, it is expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

10 strains demonstrated mean THCA titers greater than the mean THCA titer of the t807949 positive control (FIG. 13A, Table 14). The t807949 positive control expresses a THCAS corresponding to the sequence associated with Uniprot Accession I1VOCS, except that instead of its endogenous signal peptide, it is expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16. The strains that demonstrated mean THCA titers greater than the mean THCA titer of the t807949 positive control included: strain t826279, which expresses a TS that includes amino acid substitutions R31Q, H56N, I74T, N90V, A250P, S255V, Q475K, T492N, H494E, and A495E relative to SEQ ID NO: 14; strain t825084, which expresses a TS that includes amino acid substitutions R31Q, M61S, I74T, N90V, A250P, S255V, T492N, and H494E relative to SEQ ID NO: 14; strain t826132, which expresses a TS that includes amino acid substitutions R31Q, K40Q, H41Y, I74T, N90V, V129I, V288L, K296R, F345L, F360Y, A411V, E424D, H494P, and A495E relative to SEQ ID NO: 14; strain t825263, which expresses a TS that includes amino acid substitutions R31Q, K40Q, H41Y, I74T, N90V, V129I, V288L, K296R, F345L, F360Y, A411V, E424D, H494P, and A495E relative to SEQ ID NO: 14; strain t825221, which expresses a TS that includes amino acid substitutions R31Q, K40Q, H41Y, I74T, N90V, V129I, V288L, K296R, F345L, F360Y, A411V, E424D, H494P, and A495E relative to SEQ ID NO: 14; strain t825099, which expresses a TS that includes amino acid substitutions R31Q, K40Q, H41Y, I74T, N90V, V129I, V288L, K296R, F345L, F360Y, A411V, E424D, H494P, A495E, and Y419F relative to SEQ ID NO: 14; strain t825085, which expresses a TS that includes amino acid substitutions R31Q, K40Q, H41Y, H56N, I74T, N90V, V1291, V288L, K296R, F345L, F360Y, A411V, E424D, Q475K, H494P, and A495E relative to SEQ ID NO: 14; strain t825496, which expresses a TS that includes amino acid substitutions R31Q, K40Q, H41 Y, 174T, N90V, V1291, S255V, V288L, K296R, T340E, F345L, F360Y, A411V, E424D, Q475K, T492N, H494P, and A495E relative to SEQ ID NO: 14; strain t825914, which expresses a TS that includes amino acid substitutions R31Q, K40Q, H41Y, V46P, V52L, H56N, M61S, 174T, N90V, V129I, S255V, V288L, K296R, T340E, F345L, F360Y, A411V, E424D, Q475K, T492N, H494P, and A495E relative to SEQ ID NO: 14; and strain t826054, which expresses a TS that includes amino acid substitutions R31Q, K40Q, H41Y, I74T, N90V, V1291, V288L, K296R, F345L, F360Y, A411V, E424D, H494P, and A495E relative to SEQ ID NO: 14.

98 strains demonstrated mean CBDA titers greater than the mean CBDA titer of the t807973 positive control, with 50 of these strains demonstrating titers greater than 10-fold higher (FIG. 13B, Table 14), including: strain t826093, which expresses a TS that includes amino acid substitutions S100A, S116A, and H213N relative to SEQ ID NO: 13; strain t826274, which expresses a TS that includes amino acid substitutions H69Q, G95A, S116A, T339E, and Q343E relative to SEQ ID NO: 13; strain t825987, which expresses a TS that includes amino acid substitutions H69Q, G95A, S116A, and T339E relative to SEQ ID NO: 13; strain t826072, which expresses a TS that includes amino acid substitution Si 16A relative to SEQ ID NO: 13; strain t825341, which expresses a TS that includes amino acid substitutions T47A, L49P, N56H, N57D, P58Q, H69Q, H89N, and G95A, and an insertion at residue S253, relative to SEQ ID NO: 13: strain t825248, which expresses a TS that includes amino acid substitutions G95A, S116A, and Q343E relative to SEQ ID NO: 13; strain t824773, which expresses a TS that includes amino acid substitutions G95A, S116A, and T339E relative to SEQ ID NO: 13; strain t825766, which expresses a TS that includes amino acid substitutions K50N, H69Q, G95A, H213N, T339E, and L344M relative to SEQ ID NO: 13; strain t824918, which expresses a TS that includes amino acid substitutions H69Q, G95A, S116A, H213N, T339E, and Q343E relative to SEQ ID NO: 13; strain t825034, which expresses a TS that includes amino acid substitutions K50N, H69Q, G95A, and Q343E relative to SEQ ID NO: 13; strain t825126, which expresses a TS that includes amino acid substitutions K50N, H69Q, G95A, H213N, Q343E, and L344M relative to SEQ ID NO: 13, strain t824688, which expresses a TS that includes amino acid substitutions G95A, S116A, H213N, T339E, Q343E, and L344M relative to SEQ ID NO: 13, strain t825154, which expresses a TS that includes amino acid substitutions G95A, S116A, T339E, and Q343E relative to SEQ ID NO: 13, strain t825930, which expresses a TS that includes amino acid substitutions G95A, S116A, H213N, Q343E, and L344M relative to SEQ ID NO: 13; strain t824807, which expresses a TS that includes amino acid substitutions G95A, S116A, H213N, T339E, and Q343E relative to SEQ ID NO: 13, strain t825277, which expresses a TS that includes amino acid substitutions R31Q, T47A, L49P, N56H, N57D, P58Q, H69Q, H89N, and G95A, and an insertion at residue S253, relative to SEQ ID NO: 13, strain t824603, which expresses a TS that includes amino acid substitutions K50N, G95A, S116A, H213N, L344M, and N527D relative to SEQ ID NO: 13; strain t825936, which expresses a TS that includes amino acid substitutions G95A, S116A, T339E, Q343E, and L344M relative to SEQ ID NO: 13; strain t825123, which expresses a TS that includes amino acid substitution A414V relative to SEQ ID NO: 13; strain t825215, which expresses a TS that includes amino acid substitution at A414V relative to SEQ ID NO: 13; strain t825585, which expresses a TS that includes amino acid substitution A414V relative to SEQ ID NO: 13; strain t825012, which expresses a TS that includes amino acid substitutions K50N, H69Q, H213N, T339E, Q343E, and A414V relative to SEQ ID NO: 13; strain t825773, which expresses a TS that includes amino acid substitutions G95A, A414V, and N527D relative to SEQ ID NO: 13; strain t824786, which expresses a TS that includes amino acid substitutions H69Q, G95A, H213N, S322E, Q343E, L344M, and A414V relative to SEQ ID NO: 13; strain t826099, which expresses a TS that includes amino acid substitutions G95A, Q343E, G378T, and A414V relative to SEQ ID NO: 13; strain t824942, which expresses a TS that includes amino acid substitutions K50N, H69Q, G95A, T339E, Q343E, L344M, and A414V relative to SEQ ID NO: 13; strain t824626, which expresses a TS that includes amino acid substitutions H69Q, G95A, H213N, L344M, and A414V relative to SEQ ID NO: 13; strain t824654, which expresses a TS that includes amino acid substitutions K50N, H69Q, G95A, Q343E, and A414V relative to SEQ ID NO: 13; strain t824746, which expresses a TS that includes amino acid substitutions H69Q, G95A, H213N, T339E, Q343E, L344M, and A414V relative to SEQ ID NO: 13; strain t825877, which expresses a TS that includes amino acid substitutions G95A, H213N, Q343E, and A414V relative to SEQ ID NO: 13; strain t825841, which expresses a TS that includes amino acid substitutions G95A, H213N, T339E, Q343E, A414V, and D492N relative to SEQ ID NO: 13; strain t824646, which expresses a TS that includes amino acid substitutions H69Q, G95A, H213N, Q343E, A414V, and N527D relative to SEQ ID NO: 13; strain t824659, which expresses a TS that includes amino acid substitutions G95A, H213N, T339E, G378T, A410V, A414V, and I445V relative to SEQ ID NO: 13; strain t824540, which expresses a TS that includes amino acid substitutions H69Q, G95A, Q343E, A414V, and N527D relative to SEQ ID NO: 13; strain t825933, which expresses a TS that includes amino acid substitutions K50N, H69Q, G95A, H213N, Q343E, L344M, and A414V relative to SEQ ID NO: 13; strain t826097, which expresses a TS that includes amino acid substitutions K50N, G95A, and A414V relative to SEQ ID NO: 13; strain t824571, which expresses a TS that includes amino acid substitutions K50N, H69Q, G95A, H213N, T339E, Q343E, and A414V relative to SEQ ID NO: 13; strain t825593, which expresses a TS that includes amino acid substitutions G95A, T339E, L344M, and A414V relative to SEQ ID NO: 13; strain t825108, which expresses a TS that includes amino acid substitutions K50N, H213N, Q343E, L344M, and A414V relative to SEQ ID NO: 13; strain t825724, which expresses a TS that includes amino acid substitutions G95A, H213N, T339E, Q343E, and A414V relative to SEQ ID NO: 13; strain t824539, which expresses a TS that includes amino acid substitutions K50N, G95A, H213N, T339E, Q343E, A414V, and D492N relative to SEQ ID NO: 13; strain t824653, which expresses a TS that includes amino acid substitutions K50N, G95A, H213N, T339E, Q343E, L344M, and A414V relative to SEQ ID NO: 13; strain t825833, which expresses a TS that includes amino acid substitutions G95A, T339E, Q343E, L344M, and A414V relative to SEQ ID NO: 13; strain t825739, which expresses a TS that includes amino acid substitutions G95A, Q343E, L344M, and A414V relative to SEQ ID NO: 13; strain t825907, which expresses a TS that includes amino acid substitutions G95A, H213N, Q343E, L344M, and A414V relative to SEQ ID NO: 13; strain t824625, which expresses a TS that includes amino acid substitutions G95A, T339E, Q343E, and A414V relative to SEQ ID NO: 13; strain t825889, which expresses a TS that includes amino acid substitutions G95A, H213N, T339E, L344M, and A414V relative to SEQ ID NO: 13, strain t824612, which expresses a TS that includes amino acid substitutions K50N, G95A, H213N, Q343E, L344M, and A414V relative to SEQ ID NO: 13; strain t825862, which expresses a TS that includes amino acid substitutions K50N, G95A, Q343E, L344M, and A414V relative to SEQ ID NO: 13; and strain t825935, which expresses a TS that includes amino acid substitutions G95A, H213N, T339E, Q343E, L344M, and A414V relative to SEQ ID NO: 13.

11 strains demonstrated mean CBCA titers greater than 71000.00 μg/L (FIG. 13C, Table 14), including: strain t824932, which expresses a TS that includes amino acid substitutions R31Q, K40Q, H41 Y, H56N, M61S, I74T, N90V, V129I, S255V, V288L, M290F, K296R, T340E, F345L, T351I, F360Y, A411V, E424D, Q475K, T492N, H494P, and A495E relative to SEQ ID NO: 14, strain t824618, which expresses a TS that includes amino acid substitutions R31Q, K40Q, H41Y, V46P, H56N, Q58S, M61S, I74T, N90V, V129I, S255V, V288L, M290F, K296R, T340E, F345L, F360Y, A411V, E424D, T4461, Q475K, T492N, H494P, and A495E relative to SEQ ID NO: 14; strain t825910, which expresses a TS that includes amino acid substitutions R31Q, K40Q, H41Y, V46P, M61S, I74T, N90V, V129I, S255V, V288L, M290F, K296R, T340E, F345L, F360Y, A411V, E424D, T492N, H494P, and A495E relative to SEQ ID NO: 14; strain t824996, which expresses a TS that includes amino acid substitutions R31Q, K40Q, H41Y, V46P, M61S, I74T, N90V, V1291, S255V, V288L, M290F, K296R, T340E, F345L, F360Y, A411V, E424D, T492N, H494P, and A495E relative to SEQ ID NO: 14; strain t825528, which expresses a TS that includes amino acid substitutions R31Q, K40Q, H41Y, V46P, H56N, Q58S, M61S, I74T, N90V, V129I, V158L, S255V, V288L, M290F, K296R, T340E, F345L, F360Y, A411V, E424D, Q475K, T492N, H494P, and A495E relative to SEQ ID NO: 14; strain t825043, which expresses a TS that includes amino acid substitutions R31Q, K40Q, H41Y, Q58S, M61S, I74T, N90V, V129I, H143E, S255V, V288L, M290F, K296R, T340E, F345L, F360Y, A411V, E424D, Q475K, T492N, H494P, and A495E relative to SEQ ID NO: 14; strain t825978, which expresses a TS that includes amino acid substitutions R31Q, K40Q, H41Y, M61S, 174T, N90V, V1291, S255V, V288L, M290F, K296R, T340E, F345L, F360Y, A411V, E424D, Q475K, T492N, H494P, and A495E relative to SEQ ID NO: 14; strain t824498, which expresses a TS that includes amino acid substitutions R31Q, K40Q, H41Y, V46P, H56N, M61S, I74T, N90V, V129I, H143E, S255V, V288L, M290F, K296R, T340E, F345L, Y354F, F360Y, A411V, E424D, Q475K, T492N, H494P, and A495E relative to SEQ ID NO: 14; strain t825259, which expresses a TS that includes amino acid substitutions R31Q, K40Q, H41Y, H56N, Q58S, M61S, I74T, N90V, V1291, S255V, V288L, M290F, K296R, T340E, F345L, F360Y, A411V, E424D, Q475K, T492N, H494P, and A495E relative to SEQ ID NO: 14, strain t825077, which expresses a TS that includes amino acid substitutions R31Q, K40Q, H41Y, I74T, N90V, V129I, H143E, S255V, V288L, M290F, K296R, F345L, F360Y, A411V, E424D, T492N, H494P, and A495E relative to SEQ ID NO: 14; and strain t825269, which expresses a TS that includes amino acid substitutions R31Q, K40Q, H41Y, V46P, V52I, H56N, Q58S, M61S, I74T, N90V, V129I, S255V, V288L, M290F, K296R, T340E, F345L, F360Y, A411V, E424D, Q475K, T492N, H494P, and A495E relative to SEQ ID NO: 14. This level of production of CBCA is particularly surprising since other annotated C. sativa CBCASs reported in the literature failed to demonstrate CBCAS activity when screened in Example 2. Significantly, these candidate terminal synthases represent the first C. sativa-derived terminal synthases with significant CBCAS activity.

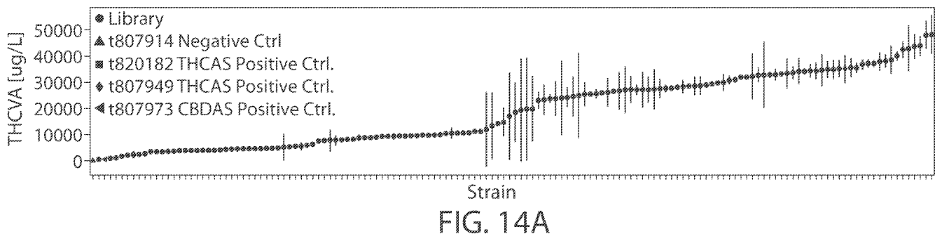

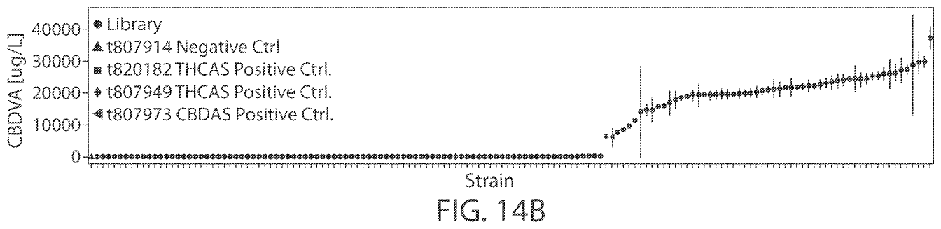

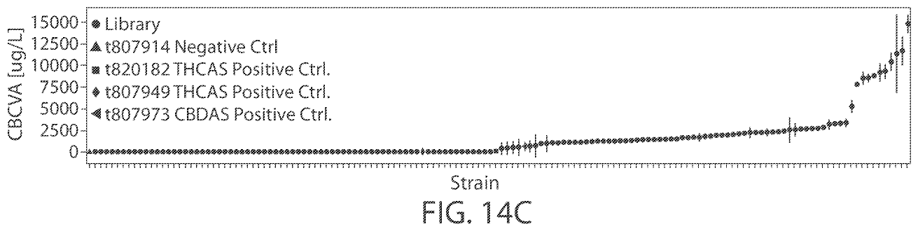

Similarly, 73 strains demonstrated mean THCVA titers greater than the mean THCVA titer of the t820182 positive control. 15 strains demonstrated mean THCVA titers greater than the mean THCVA titer of the t807949 positive control (FIG. 14A, Table 15), including: strain t825377, which expresses a TS that includes amino acid substitutions R31Q, K40Q, H41Y, N44T, A47T, P49A, L59F, I74T, V85I, S88L, N90V, A95G, P542L, and H543R relative to SEQ ID NO: 14; strain t825213, which expresses a TS that includes amino acid substitutions R31Q, K40Q, H41Y, I74T, N90V, V1291, V288L, K296R, F345L, F360Y, A411V, E424D, H494P, and A495E relative to SEQ ID NO: 14; strain t825219, which expresses a TS that includes amino acid substitutions R31Q, K40Q, H41Y, N44T, A47T, P49A, L59F, 174T, V85I, S88L, N90V, A95G, P542L, and H543R relative to SEQ ID NO: 14; strain t825379, which expresses a TS that includes amino acid substitutions R31Q, K40Q, H41Y, 174T, N90V, V1291, V288L, K296R, F345L, F360Y, A411V, E424D, H494P, and A495E relative to SEQ ID NO: 14; strain t825151, which expresses a TS that includes amino acid substitutions R31Q, K40Q, H41Y, I74T, N90V, V129I, V288L, K296R, F345L, F360Y, A411V, E424D, H494P, and A495E relative to SEQ ID NO: 14; strain t826100, which expresses a TS that includes amino acid substitutions R31Q, K40Q, H41Y, I74T, N90V, V129I, V288L, K296R, F345L, F360Y, A411V, E424D, H494P, and A495E relative to SEQ ID NO: 14; strain t825129, which expresses a TS that includes amino acid substitutions R31Q, K40Q, H41Y, I74T, N90V, V1291, V288L, K296R, F345L, F360Y, A411V, E424D, H494P, and A495E relative to SEQ ID NO: 14; strain t825297, which expresses a TS that includes amino acid substitutions R31Q, 174T, N90V, A250D, S255V, T492N, and H494E relative to SEQ ID NO: 14; strain t824990, which expresses a TS that includes amino acid substitutions I74T, N90V, A250P, and H494E relative to SEQ ID NO: 14, strain t825049, which expresses a TS that includes amino acid substitutions M61S, N90V, A250D, S255V, Q475K, T492N, and A495E relative to SEQ ID NO: 14; strain t825086, which expresses a TS that includes amino acid substitutions R31Q, K40Q, H41Y, V46P, H56N, Q58S, M61S, I74T, N90V, V1291, H143E, S255V, V288L, K296R, T340E, F345L, Y354F, F360Y, A411V, E424D, Q475K, T492N, H494P, and A495E relative to SEQ ID NO: 14; strain t825058, which expresses a TS that includes amino acid substitutions R31Q I74T, N90V, A250D, and S255V relative to SEQ ID NO: 14, strain t826279, which expresses a TS that includes amino acid substitutions R31Q, H56N, 174T, N90V, A250P, S255V, Q475K, T492N, H494E, and A495E relative to SEQ ID NO: 14; strain t826132, which expresses a TS that includes amino acid substitutions R31Q, K40Q, H41Y, I74T, N90V, V1291, V288L, K296R, F345L, F360Y, A411V, E424D, H494P, and A495E relative to SEQ ID NO: 14; and strain t825085, which expresses a TS that includes amino acid substitutions R31Q, K40Q, H41Y, H56N, 174T, N90V, V1291, V288L, K296R, F345L, F360Y, A411V, E424D, Q475K, H494P, and A495E relative to SEQ ID NO: 14.

55 strains demonstrated mean CBDVA titers greater than the mean CBDVA titer of the t807973 positive control, with 50 of these strains demonstrating titers greater than 10-fold higher (FIG. 14B, Table 15), including: strain t826093, which expresses a TS that includes amino acid substitutions S100A, S116A, and H213N relative to SEQ ID NO: 13; strain t826274, which expresses a TS that includes amino acid substitutions H69Q, G95A, S116A, T339E, and Q343E relative to SEQ ID NO: 13; strain t825987, which expresses a TS that includes amino acid substitutions H69Q, G95A, S116A, and T339E relative to SEQ ID NO: 13; strain t826072, which expresses a TS that includes amino acid substitution S116A relative to SEQ ID NO: 13; strain t826096, which expresses a TS that includes amino acid substitution S116A relative to SEQ ID NO: 13; strain t825125, which expresses a TS that includes amino acid substitution K50N relative to SEQ ID NO: 13; strain t825217, which expresses a TS that includes amino acid substitution K50N relative to SEQ ID NO: 13; strain t825341, which expresses a TS that includes amino acid substitutions T47A, L49P, N56H, N57D, P58Q, H69Q, H89N, and G95A, and an insertion at residue S253, relative to SEQ ID NO: 13; strain t825248, which expresses a TS that includes amino acid substitutions G95A, Si 16A, and Q343E relative to SEQ ID NO: 13; strain t824773, which expresses a TS that includes amino acid substitutions G95A, S116A, and T339E relative to SEQ ID NO: 13; strain t825766, which expresses a TS that includes amino acid substitutions K50N, H69Q, G95A, H213N, T339E, and L344M relative to SEQ ID NO: 13; strain t824918, which expresses a TS that includes amino acid substitutions H69Q, G95A, Si 16A, H213N, T339E, and Q343E relative to SEQ ID NO: 13; strain t825034, which expresses a TS that includes amino acid substitutions K50N, H69Q, G95A, and Q343E relative to SEQ ID NO: 13; strain t825126, which expresses a TS that includes amino acid substitutions K50N, H69Q, G95A, H213N, Q343E, and L344M relative to SEQ ID NO: 13; strain t824688, which expresses a TS that includes amino acid substitutions G95A, S116A, H213N, T339E, Q343E, and L344M relative to SEQ ID NO: 13; strain t825154, which expresses a TS that includes amino acid substitutions G95A, S116A, T339E, and Q343E relative to SEQ ID NO: 13; strain t825930, which expresses a TS that includes amino acid substitutions G95A, S116A, H213N, Q343E, and L344M relative to SEQ ID NO: 13; strain t824807, which expresses a TS that includes amino acid substitutions G95A, S116A, H213N, T339E, and Q343E relative to SEQ ID NO: 13; strain t825277, which expresses a TS that includes amino acid substitutions R31Q, T47A, L49P, N56H, N57D, P58Q, H69Q, H89N, and G95A, and an insertion at residue S253, relative to SEQ ID NO: 13; strain t824603, which expresses a TS that includes amino acid substitutions K50N, G95A, S116A, H213N, L344M, and N527D relative to SEQ ID NO: 13; strain t826076, which expresses a TS that includes amino acid substitution S116A relative to SEQ ID NO: 13; strain t825936, which expresses a TS that includes amino acid substitutions G95A, S116A, T339E, Q343E, and L344M relative to SEQ ID NO: 13; strain t825123, which expresses a TS that includes amino acid substitution A414V relative to SEQ ID NO: 13; strain t825215, which expresses a TS that includes amino acid substitution A414V relative to SEQ ID NO: 13; strain t825585, which expresses a TS that includes amino acid substitution A414V relative to SEQ ID NO: 13; strain t825012, which expresses a TS that includes amino acid substitutions K50N, H69Q, H213N, T339E, Q343E, and A414V relative to SEQ ID NO: 13; strain t825773, which expresses a TS that includes amino acid substitutions G95A, A414V, and N527D relative to SEQ ID NO: 13; strain t824786, which expresses a TS that includes amino acid substitutions H69Q, G95A, H213N, S322E, Q343E, L344M, and A414V relative to SEQ ID NO: 13; strain 1826099, which expresses a TS that includes amino acid substitutions G95A, Q343E, G378T, and A414V relative to SEQ ID NO: 13; strain t824942, which expresses a TS that includes amino acid substitutions K50N, H69Q, G95A, T339E, Q343E, L344M, and A414V relative to SEQ ID NO: 13; strain t826070, which expresses a TS that includes amino acid substitution S116A relative to SEQ ID NO: 13; strain t824626, which expresses a TS that includes amino acid substitutions H69Q, G95A, H213N, L344M, and A414V relative to SEQ ID NO: 13. strain t824654, which expresses a TS that includes amino acid substitutions K50N, H69Q, G95A, Q343E, and A414V relative to SEQ ID NO: 13; strain t824746, which expresses a TS that includes amino acid substitutions H69Q, G95A, H213N, T339E, Q343E, L344M, and A414V relative to SEQ ID NO: 13; strain 1825877, which expresses a TS that includes amino acid substitutions G95A, H213N, Q343E, and A414V relative to SEQ ID NO: 13; strain t825841, which expresses a TS that includes amino acid substitutions G95A, H213N, T339E, Q343E, A414V, and D492N relative to SEQ ID NO: 13; strain t824646, which expresses a TS that includes amino acid substitutions H69Q, G95A, H213N, Q343E, A414V, and N527D relative to SEQ ID NO: 13; strain t824659, which expresses a TS that includes amino acid substitutions G95A, H213N, T339E, G378T, A410V, A414V, and 1445V relative to SEQ ID NO: 13; strain t824540, which expresses a TS that includes amino acid substitutions H69Q, G95A, Q343E, A414V, and N527D relative to SEQ ID NO: 13; strain t825933, which expresses a TS that includes amino acid substitutions K50N, H69Q, G95A, H213N, Q343E, L344M, and A414V relative to SEQ ID NO: 13; strain t826097, which expresses a TS that includes amino acid substitutions K50N, G95A, and A414V relative to SEQ ID NO: 13; strain t824571, which expresses a TS that includes amino acid substitutions K50N, H69Q, G95A, H213N, T339E, Q343E, and A414V relative to SEQ ID NO: 13; strain t825593, which expresses a TS that includes amino acid substitutions G95A, T339E, L344M, and A414V relative to SEQ ID NO: 13; strain 1825108, which expresses a TS that includes amino acid substitutions K50N, H213N, Q343E, L344M, and A414V relative to SEQ ID NO: 13; strain t825724, which expresses a TS that includes amino acid substitutions G95A, H213N, T339E, Q343E, and A414V relative to SEQ ID NO: 13; strain t824539, which expresses a TS that includes amino acid substitutions K50N, G95A, H213N, T339E, Q343E, A414V, and D492N relative to SEQ ID NO: 13; strain 1824653, which expresses a TS that includes amino acid substitutions K50N, G95A, H213N, T339E, Q343E, L344M, and A414V relative to SEQ ID NO: 13; strain t825833, which expresses a TS that includes amino acid substitutions G95A, T339E, Q343E, L344M, and A414V relative to SEQ ID NO: 13; strain t825739, which expresses a TS that includes amino acid substitutions G95A, Q343E, L344M, and A414V relative to SEQ ID NO: 13; strain t825907, which expresses a TS that includes amino acid substitutions G95A, H213N, Q343E, L344M, and A414V relative to SEQ ID NO: 13; strain t824625, which expresses a TS that includes amino acid substitutions G95A, T339E, Q343E, and A414V relative to SEQ ID NO: 13; strain t825889, which expresses a TS that includes amino acid substitutions G95A, H213N, T339E, L344M, and A414V relative to SEQ ID NO: 13; strain t824612, which expresses a TS that includes amino acid substitutions K50N, G95A, H213N, Q343E, L344M, and A414V relative to SEQ ID NO: 13; strain t825862, which expresses a TS that includes amino acid substitutions K50N, G95A, Q343E, L344M, and A414V relative to SEQ ID NO: 13; and strain t825935, which expresses a TS that includes amino acid substitutions G95A, H213N, T339E, Q343E, L344M, and A414V relative to SEQ ID NO: 13.

11 strains demonstrated mean CBCVA titers greater than 5000.00 μg/L (FIG. 14C, Table 15), including: strain t825341, which expresses a TS that includes amino acid substitutions T47A, L49P, N56H, N57D, P58Q, H69Q, H89N, and G95A, and an insertion at residue S253, relative to SEQ ID NO: 13; strain t824932, which expresses a TS that includes amino acid substitutions R31Q, K40Q, H41Y, H56N, M61S, 174T, N90V, V1291, S255V, V288L, M290F, K296R, T340E, F345L, T351I, F360Y, A411V, E424D, Q475K, T492N, H494P, and A495E relative to SEQ ID NO: 14; strain t824618, which expresses a TS that includes amino acid substitutions R31Q, K40Q, H41Y, V46P, H56N, Q58S, M61S, 174T, N90V, V129I, S255V, V288L, M290F, K296R, T340E, F345L, F360Y, A411V, E424D, T446I, Q475K, T492N, H494P, and A495E relative to SEQ ID NO: 14; strain t825910, which expresses a TS that includes amino acid substitutions R31Q, K40Q, H41Y, V46P, M61S, I74T, N90V, V1291, S255V, V288L, M290F, K296R, T340E, F345L, F360Y, A411V, E424D, T492N, H494P, and A495E relative to SEQ ID NO: 14; strain t824996, which expresses a TS that includes amino acid substitutions R31Q, K40Q, H41Y, V46P, H56N, M61S, I74T, N90V, V1291, S255V, V288L, M290F, K296R, F345L, F360Y, A411V, E424D, T492N, H494P, and A495E relative to SEQ ID NO: 14; strain t825528, which expresses a TS that includes amino acid substitutions R31Q, K40Q, H41Y, V46P, H56N, Q58S, M61S, I74T, N90V, V1291, V158L, S255V, V288L, M290F, K296R, T340E, F345L, F360Y, A411V, E424D, Q475K, T492N, H494P, and A495E relative to SEQ ID NO: 14; strain t825043, which expresses a TS that includes amino acid substitutions R31Q, K40Q, H41Y, Q58S, M61S, I74T, N90V, V129I, H143E, S255V, V288L, M290F, K296R, T340E, F345L, F360Y, A411V, E424D, Q475K, T492N, H494P, and A495E relative to SEQ ID NO: 14; strain t825978, which expresses a TS that includes amino acid substitutions R31Q, K40Q, H41Y, M61S, I74T, N90V, V1291, S255V, V288L, M290F, K296R, T340E, F345L, F360Y, A411V, E424D, Q475K, T492N, H494P, and A495E relative to SEQ ID NO: 14; strain t824498, which expresses a TS that includes amino acid substitutions R31Q, K40Q, H41Y, V46P, H56N, M61S, I74T, N90V, V1291, H143E, S255V, V288L, M290F, K296R, T340E, F345L, Y354F, F360Y, A411V, E424D, Q475K, T492N, H494P, and A495E relative to SEQ ID NO: 14; strain t825259, which expresses a TS that includes amino acid substitutions R31Q, K40Q, H41Y, H56N, Q58S, M61S, 174T, N90V, V129I, S255V, V288L, M290F, K296R, T340E, F345L, F360Y, A411V, E424D, Q475K, T492N, H494P, and A495E relative to SEQ ID NO: 14; and strain t825269, which expresses a TS that includes amino acid substitutions R31Q, K40Q, H41Y, V46P, V52I, H56N, Q58S, M61S, I74T, N90V, V129L, S255V, V288L, M290F, K296R, T340E, F345L, F360Y, A411V, E424D, Q475K, T492N, H494P, and A495E relative to SEQ ID NO: 14.

Table 14 shows THCA, CBDA, and CBCA activity data for the 142 strains elevated to a secondary assay. Table 15 shows THCVA, CBDVA, and CBCVA activity data for the 142 strains elevated to a secondary assay. Sequence data for strains in Table 14 and Table 15 are shown in Table 23.

Increased titer of C6 and/or C4 terminal products observed among the terminal synthase candidates in this library may be the result of one or more mutations acting alone or in combination to increase solubility and/or increase stability of the terminal synthase enzymes. For example, some of the hit THCAS candidates comprised one or more of the following mutations: V52I, V288L, T340E, F345L, F360Y, Y419F, E424D, T492N, K40Q, H56N, A250D, Q475K, and H494E, all of which were mapped to the surface of the tertiary structure of C. sativa THCAS (Uniprot Accession No. Q8GTB6), indicating that the mutations may contribute to increased solubilization or stability of the enzyme, which may result in increased THCA and/or THCVA titer. Similarly, some of the hit CBDAS candidates comprised one or more of the following mutations, S322E, T339E, K50N, H213N, L344M, and N527D, all of which were mapped to the surface of the tertiary structure of C. sativa CBDAS (Uniprot Accession No. A6P6V9), indicating that the mutations may contribute to increased solubilization or stability of the enzyme, which may result in increased CBDA and/or CBDVA titer. Also, some of the hit CBCAS candidates comprised one or more of the following mutations of H41Y, M61S, H56N, Q58S, V52I, H143E, T340E, F345L, A411V, E424D, T492N, Q475K, Y354F, and H494P, all of which were mapped to the surface of the tertiary structure of C. sativa THCAS (Uniprot Accession No. Q8GTB6), indicating that the mutations may contribute to increased solubilization or stability of the enzyme, which may result in increased CBCA and/or CBCVA titer.

Without wishing to be bound by any theory, one or more mutations described herein may have an effect on the selectivity of terminal synthase substrates. For example, the following mutations were found to be unique among the THCVA hits relative to the THCA hits. L59F, A47T, P49A, S88L, H143E, A250D, Y354F, P542L, H543R, N44T, Q58S, and A95G. Interestingly, mutation Y354F was mapped to within 6 angstroms of the catalytic trio of C. sativa THCAS (Uniprot Accession No. Q8GTB6) and to within approximately 8.4 angstroms of the location of the C6 carbon of THCA. Based on proximity to the active site, the residue at amino acid 354 may interact directly with THCA and/or THCVA. The mutation Y354F, which changes the residue from polar to nonpolar, may alter the hydrophobicity of the binding pocket and may affect the binding of terminal synthase substrates. Similarly, the mutation T4461 was found to be unique among the CBCVA hits relative to the CBCA hits. Based on a generated comparative model, the residue at position 446 is predicted to be within 4 angstroms of the substrate binding site of C. sativa THCAS (Uniprot Accession No. Q8GTB6). The mutation T446I, which changes the residue from an uncharged polar residue to a bulkier hydrophobic residue, may alter the hydrophobicity of the binding pocket and may affect the binding of terminal synthase substrates.

Since product promiscuity has previously been noted among the C. sativa terminal synthases and observed among the terminal synthase candidates in this library, correlating template/mutations to changes in product profile may indicate critical residues for determining product specificity. The CBCAS hits identified here provide examples of this. Each CBCAS hit strain was derived from three putative THCAS templates; two that were derived from C. sativa RNAseq data and one from a previously engineered ancestral reconstruction. These hits range in their production of CBCA as a percentage of all terminal products measured (Percent Product CBCA) from approximately 41% to 100%. One mutation that may contribute to a shift in activity from THCA to CBCA is the V158L amino acid substitution. The V158 residue was mapped to the outer second-shell (approximately 15 angstroms from the active site) of the C. sativa THCAS (Uniprot Accession No. Q8GTB6) tertiary structure, indicating that the mutation may contribute to increased solubilization or stability of the enzyme.

The CBDAS hits demonstrate the most product promiscuity, evaluated as the percentage of total cannabinoids generated that is not CBDA. For example, the top 10 CBDAS range in their production of CBDA as a percentage of C6 terminal products (e.g. THCA, CBDA, and CBCA) measured (Percent Product CBDA) from approximately 64-71%.

TABLE 14
Terminal C6 Product Titers of terminal synthase candidate enzymes in S. cerevisiae

| | | Mean | Std Dev | Mean | Std Dev | Mean | Std Dev | % | % | % |
|---|---|---|---|---|---|---|---|---|---|---|
| Strain | Strain | THCA | THCA | CBDA | CBDA | CBCA | CBCA | Product | Product | Product |
| ID | Type | [μg/L] | [μg/L] | [μg/L] | [μg/L] | [μg/L] | [μg/L] | THCA | CBDA | CBCA |
| t820182 | Positive | 18331.58 | 5551.4 | 0 | 0 | 2780.5 | 5654.16 | 86.83 | 0 | 13.17 |
| | Control | | | | | | | | | |
| | C. sativa | | | | | | | | | |
| | THCAS; | | | | | | | | | |
| | based on | | | | | | | | | |
| | UniProt | | | | | | | | | |
| | Q8GTB6 | | | | | | | | | |
| t807973 | Positive | 0 | 0 | 1353.01 | 1088.55 | 0 | 0 | 0 | 100 | 0 |
| | Control | | | | | | | | | |
| | C. sativa | | | | | | | | | |
| | CBDAS: | | | | | | | | | |
| | based on | | | | | | | | | |
| | UniProt | | | | | | | | | |
| | A6P6V9 | | | | | | | | | |
| t807949 | Positive | 179871.9 | 22612.45 | 572.77 | 687.71 | 0 | 0 | 99.68 | 0.32 | 0 |
| | Control | | | | | | | | | |
| | C. sativa | | | | | | | | | |
| | THCAS: | | | | | | | | | |
| | based on | | | | | | | | | |
| | UniProt | | | | | | | | | |
| | I1V0C5 | | | | | | | | | |
| t807914 | GFP | 185.76 | 788.13 | 0 | 0 | 112.35 | 476.68 | 62.31 | 0 | 37.69 |
| | negative | | | | | | | | | |
| | control | | | | | | | | | |
| t826093 | Library | 0 | 0 | 34334.05 | 3304.61 | 5044.43 | 512.95 | 0 | 87.19 | 12.81 |
| t826274 | Library | 0 | 0 | 20894.82 | 3946.51 | 9146.36 | 1486.94 | 0 | 69.55 | 30.45 |
| t825987 | Library | 0 | 0 | 19997.88 | 1302.17 | 9508.21 | 307.27 | 0 | 67.78 | 32.22 |
| t826072 | Library | 0 | 0 | 15787.12 | 2075.94 | 2277.92 | 1612.75 | 0 | 87.39 | 12.61 |
| t826096 | Library | 0 | 0 | 8612.72 | 1079.62 | 0 | 0 | 0 | 100 | 0 |
| t825125 | Library | 0 | 0 | 2907.54 | 531.8 | 0 | 0 | 0 | 100 | 0 |
| t825217 | Library | 0 | 0 | 2575.18 | 499.68 | 0 | 0 | 0 | 100 | 0 |
| t825189 | Library | 0 | 0 | 2322.66 | 730.6 | 0 | 0 | 0 | 100 | 0 |
| t825341 | Library | 874.4 | 1748.8 | 19511.89 | 5608.99 | 5253.05 | 2116.14 | 3.41 | 76.1 | 20.49 |
| t825248 | Library | 2270.53 | 1876.34 | 28530.73 | 10073.78 | 4571.34 | 1673.78 | 6.42 | 80.66 | 12.92 |
| t824773 | Library | 2506.6 | 72.57 | 31384.4 | 1807.27 | 4940.11 | 213.91 | 6.46 | 80.82 | 12.72 |
| t825766 | Library | 2708.12 | 3135.13 | 31210.55 | 8037.03 | 7539.44 | 2792.47 | 6.53 | 75.28 | 18.19 |
| t824918 | Library | 2942.18 | 1167.41 | 24067.74 | 7915.22 | 10025.72 | 2933.76 | 7.94 | 64.99 | 27.07 |
| t825034 | Library | 3016.39 | 623.49 | 26421.46 | 1136.01 | 5768.93 | 357.68 | 8.57 | 75.05 | 16.39 |
| t824932 | Library | 3086.6 | 93.19 | 6286.93 | 359.65 | 71623.65 | 1280.02 | 3.81 | 7.76 | 88.43 |
| t824618 | Library | 3511.33 | 925.7 | 7020.75 | 1776.77 | 82634.08 | 12238.37 | 3.77 | 7.54 | 88.7 |
| t825126 | Library | 3663.4 | 436.82 | 27222.4 | 3933.86 | 6702.8 | 1170.07 | 9.75 | 72.42 | 17.83 |
| t825910 | Library | 3812.23 | 2571.69 | 7892.58 | 1037.84 | 104479.6 | 13503.13 | 3.28 | 6.79 | 89.93 |
| t824688 | Library | 3887.4 | 479.19 | 47259.26 | 7166.47 | 7028.42 | 973.64 | 6.68 | 81.24 | 12.08 |
| t824996 | Library | 4019.47 | 506.1 | 6351.29 | 341.45 | 77120.13 | 6795.12 | 4.59 | 7.26 | 88.15 |
| t825528 | Library | 4359.14 | 2968.72 | 8124.85 | 908.41 | 93747.57 | 3886.43 | 4.1 | 7.65 | 88.25 |
| t825154 | Library | 4669.17 | 2088.18 | 48091.14 | 20690.96 | 7446.43 | 2922.36 | 7.76 | 79.88 | 12.37 |
| t825930 | Library | 4713.96 | 637.8 | 49322.15 | 6471.21 | 8886.39 | 834.67 | 7.49 | 78.39 | 14.12 |
| t825043 | Library | 5076.72 | 787.61 | 7859.78 | 1163.31 | 79846.68 | 7884.93 | 5.47 | 8.47 | 86.06 |
| t824807 | Library | 5276.46 | 832.07 | 65039.56 | 8938.99 | 9300.73 | 854.28 | 6.63 | 81.69 | 11.68 |
| t825978 | Library | 5374.33 | 965.67 | 8730.75 | 2181.61 | 114494.1 | 28085.4 | 4.18 | 6.79 | 89.03 |
| t825277 | Library | 5745.89 | 1606.75 | 39692.98 | 12567.82 | 12523.52 | 4387.66 | 9.91 | 68.48 | 21.61 |
| t824498 | Library | 5865.53 | 190.4 | 8266.22 | 525.63 | 100691.4 | 5784.06 | 5.11 | 7.2 | 87.69 |
| t824603 | Library | 5891.59 | 1556.45 | 48545.08 | 3622.06 | 7914.23 | 469.91 | 9.45 | 77.86 | 12.69 |
| t826076 | Library | 6188.95 | 8623.87 | 11806.36 | 2391.07 | 1311.31 | 1539.51 | 32.06 | 61.15 | 6.79 |
| t825259 | Library | 7070.85 | 3956.32 | 6734.56 | 1878.45 | 98822.68 | 31872.8 | 6.28 | 5.98 | 87.74 |
| t825936 | Library | 7285.23 | 1403.68 | 66600.94 | 13840.69 | 12655.6 | 2441.4 | 8.42 | 76.96 | 14.62 |
| t825077 | Library | 9676.18 | 8141.47 | 6914.54 | 613.25 | 93157.43 | 5728.56 | 8.82 | 6.3 | 84.88 |
| t825123 | Library | 13129.39 | 1680.16 | 38195.16 | 4131.58 | 3596.6 | 392.23 | 23.91 | 69.55 | 6.55 |
| t825215 | Library | 14458.48 | 2300.77 | 43420.83 | 8116.68 | 4892.32 | 974.65 | 23.03 | 69.17 | 7.79 |
| t825585 | Library | 16084.38 | 11952.6 | 16951.33 | 10430.92 | 10410.56 | 9537.7 | 37.02 | 39.02 | 23.96 |
| t825012 | Library | 16423.12 | 1279.78 | 38469.89 | 3258.45 | 8045.54 | 680.21 | 26.09 | 61.12 | 12.78 |
| t825773 | Library | 16908.56 | 19673.89 | 53659.46 | 63109.23 | 4715.74 | 5448.4 | 22.46 | 71.28 | 6.26 |
| t824786 | Library | 17024.85 | 1036.46 | 46909.53 | 3359.46 | 8844.55 | 469.07 | 23.39 | 64.45 | 12.15 |
| t826099 | Library | 17152.32 | 5378.28 | 58117.76 | 17254.5 | 10073.05 | 2895.87 | 20.1 | 68.1 | 11.8 |
| t824942 | Library | 17916.44 | 1512.96 | 44977.35 | 3617.75 | 9783.52 | 553.09 | 24.65 | 61.89 | 13.46 |
| t826070 | Library | 18913.79 | 34013.4 | 11147 | 1763.31 | 676.55 | 1353.1 | 61.53 | 36.27 | 2.2 |
| t824626 | Library | 20043.6 | 5668.51 | 41061.76 | 6019.68 | 8193.21 | 946.63 | 28.92 | 59.25 | 11.82 |
| t825269 | Library | 20540.8 | 826.88 | 10446.93 | 5014.16 | 87267.02 | 13690.06 | 17.37 | 8.83 | 73.8 |
| t824654 | Library | 21642.98 | 4153.67 | 55581.21 | 12452.82 | 11551.9 | 1723.82 | 24.38 | 62.61 | 13.01 |
| t824746 | Library | 25725.27 | 2802.84 | 74018.27 | 13387.34 | 15503.72 | 547.2 | 22.32 | 64.23 | 13.45 |
| t825877 | Library | 26212.74 | 19006.22 | 87869.41 | 64589.5 | 7634.39 | 5552.05 | 21.54 | 72.19 | 6.27 |
| t825841 | Library | 27217.96 | 13698.75 | 95039.89 | 49146.67 | 7691.8 | 4032.81 | 20.95 | 73.14 | 5.92 |
| t824646 | Library | 27813.3 | 3601.11 | 66902.36 | 12294.48 | 14156.8 | 1878.16 | 25.55 | 61.45 | 13 |
| t824659 | Library | 27816.71 | 2441.49 | 39731.58 | 4128.54 | 10675.74 | 713.97 | 35.56 | 50.79 | 13.65 |
| t824540 | Library | 27992.53 | 3829.52 | 63702.17 | 11554.57 | 13595.2 | 2138.63 | 26.59 | 60.5 | 12.91 |
| t825933 | Library | 29112.7 | 4909.41 | 73534.48 | 13378.36 | 15729.94 | 2349.67 | 24.59 | 62.12 | 13.29 |
| t826097 | Library | 30722.45 | 1543.08 | 92485.52 | 7768.6 | 9286.46 | 549.98 | 23.19 | 69.8 | 7.01 |
| t824571 | Library | 31144.98 | 2304.24 | 80291.14 | 5071.14 | 16132.56 | 1083.31 | 24.41 | 62.94 | 12.65 |
| t825593 | Library | 31819.54 | 4889.17 | 98484.76 | 15785.84 | 8831.07 | 1173.13 | 22.87 | 70.78 | 6.35 |
| t825108 | Library | 32594.21 | 10397.79 | 73969.64 | 8913.77 | 8527.07 | 687.94 | 28.32 | 64.27 | 7.41 |
| t825724 | Library | 33242.48 | 4916.65 | 94219.55 | 10637.63 | 9569.2 | 1611.95 | 24.26 | 68.76 | 6.98 |
| t824539 | Library | 33886.66 | 3579.31 | 100147.5 | 9518.53 | 12672.6 | 1714.52 | 23.1 | 68.26 | 8.64 |
| t824653 | Library | 36504.14 | 3083.62 | 114771.8 | 21926.16 | 14706.14 | 2111.64 | 21.99 | 69.15 | 8.86 |
| t825833 | Library | 38219.52 | 9569.95 | 71186.67 | 59450.93 | 13237.94 | 4154 | 31.16 | 58.04 | 10.79 |
| t825739 | Library | 40026.35 | 6779.27 | 11019.67 | 19696.98 | 11320.59 | 1787.31 | 24.78 | 68.21 | 7.01 |
| t825907 | Library | 40214.85 | 4318.7 | 111856.3 | 18211.98 | 14294.25 | 2784.41 | 24.17 | 67.24 | 8.59 |
| t824625 | Library | 40280.69 | 4883.92 | 123546.5 | 14017.51 | 14157.72 | 1393.13 | 22.63 | 69.41 | 7.95 |
| t825889 | Library | 41737.82 | 11404.51 | 126307.3 | 37393.32 | 12081.97 | 3166.52 | 23.17 | 70.12 | 6.71 |
| t824612 | Library | 44055.66 | 2420.56 | 123951.9 | 6769.44 | 15248.22 | 717.28 | 24.04 | 67.64 | 8.32 |
| t825862 | Library | 48078.31 | 27844.96 | 115099.2 | 46666.34 | 15307.49 | 9863.65 | 26.94 | 64.49 | 8.58 |
| t825935 | Library | 55811.38 | 16473.06 | 155541.2 | 44512.82 | 19120.6 | 5499.6 | 24.22 | 67.49 | 8.3 |
| t825105 | Library | 68573.64 | 59236.14 | 5401.56 | 5686.7 | 6518.39 | 8061.17 | 85.19 | 6.71 | 8.1 |
| t825301 | Library | 80134.31 | 94592.46 | 545.18 | 682.77 | 0 | 0 | 99.32 | 0.68 | 0 |
| t825377 | Library | 81694.66 | 61803.36 | 0 | 0 | 0 | 0 | 100 | 0 | 0 |
| t825092 | Library | 90351.7 | 104446.7 | 341.75 | 683.5 | 0 | 0 | 99.62 | 0.38 | 0 |
| t825075 | Library | 91015.05 | 107120.1 | 0 | 0 | 0 | 0 | 100 | 0 | 0 |
| t825213 | Library | 93328.31 | 39354.96 | 0 | 0 | 0 | 0 | 100 | 0 | 0 |
| t824771 | Library | 97204.76 | 74439.72 | 382.73 | 765.46 | 0 | 0 | 99.61 | 0.39 | 0 |
| t825025 | Library | 102900.6 | 72671.22 | 430.49 | 860.99 | 0 | 0 | 99.58 | 0.42 | 0 |
| t825090 | Library | 104539.6 | 127448.4 | 880.01 | 1083.83 | 0 | 0 | 99.17 | 0.83 | 0 |
| t826284 | Library | 104817.5 | 23220.67 | 1358.49 | 321.81 | 0 | 0 | 98.72 | 1.28 | 0 |
| t825024 | Library | 105084.3 | 9848.61 | 1645.15 | 226.76 | 0 | 0 | 98.46 | 1.54 | 0 |
| t825015 | Library | 111965.3 | 70206.39 | 1080.91 | 836.62 | 0 | 0 | 99.04 | 0.96 | 0 |
| t824526 | Library | 112833.9 | 16870.02 | 929.09 | 1079.14 | 0 | 0 | 99.18 | 0.82 | 0 |
| t824647 | Library | 116339.5 | 3676.66 | 814.13 | 542.99 | 0 | 0 | 99.31 | 0.69 | 0 |
| t825007 | Library | 117082.3 | 31979.17 | 782.5 | 905.21 | 0 | 0 | 99.34 | 0.66 | 0 |
| t825501 | Library | 118039.3 | 80485.26 | 1404.74 | 941.67 | 0 | 0 | 98.82 | 1.18 | 0 |
| t825219 | Library | 118135 | 43402.91 | 0 | 0 | 0 | 0 | 100 | 0 | 0 |
| t825286 | Library | 121000.2 | 85427.25 | 777.21 | 575.82 | 0 | 0 | 99.36 | 0.64 | 0 |
| t825071 | Library | 121633.8 | 44436.65 | 0 | 0 | 0 | 0 | 100 | 0 | 0 |
| t825047 | Library | 122529.8 | 8739.03 | 1701.41 | 110.97 | 0 | 0 | 98.63 | 1.37 | 0 |
| t825031 | Library | 123848.5 | 14348.04 | 1887.14 | 275.02 | 0 | 0 | 98.5 | 1.5 | 0 |
| t825633 | Library | 125217.8 | 20917.15 | 7999.45 | 8606.42 | 11851.01 | 14019.81 | 86.32 | 5.51 | 8.17 |
| t824861 | Library | 126847.3 | 18706.46 | 1393.03 | 140.62 | 0 | 0 | 98.91 | 1.09 | 0 |
| t824928 | Library | 128407.5 | 8973.23 | 3063.2 | 413.76 | 5359.34 | 10718.67 | 93.84 | 2.24 | 3.92 |
| t825725 | Library | 129276.8 | 24363.72 | 3462.05 | 6231.32 | 1977.48 | 3954.95 | 95.96 | 2.57 | 1.47 |
| t826036 | Library | 129790.4 | 15361.61 | 0 | 0 | 0 | 0 | 100 | 0 | 0 |
| t824903 | Library | 130596.7 | 15812.11 | 1927.83 | 184.37 | 0 | 0 | 98.55 | 1.45 | 0 |
| t824845 | Library | 131579.1 | 9933.63 | 620.61 | 716.62 | 0 | 0 | 99.53 | 0.47 | 0 |
| t825078 | Library | 132100.4 | 20618.15 | 4995.1 | 1022.5 | 7079.55 | 8239.81 | 91.63 | 3.46 | 4.91 |
| t826030 | Library | 132786 | 12489.09 | 0 | 0 | 0 | 0 | 100 | 0 | 0 |
| t824622 | Library | 134598 | 8953.97 | 1905.78 | 197.67 | 0 | 0 | 98.6 | 1.4 | 0 |
| t825141 | Library | 134902.8 | 13599.57 | 1428.11 | 179.1 | 0 | 0 | 98.95 | 1.05 | 0 |
| t825379 | Library | 136455.4 | 50611.98 | 1783.78 | 943.2 | 0 | 0 | 98.71 | 1.29 | 0 |
| t825625 | Library | 137718.2 | 11656.35 | 738.73 | 72.51 | 0 | 0 | 99.47 | 0.53 | 0 |
| t825309 | Library | 138669.3 | 10460.2 | 2635.69 | 1402.37 | 0 | 0 | 98.13 | 1.87 | 0 |
| t824930 | Library | 139552.7 | 31783 | 575.12 | 664.17 | 0 | 0 | 99.59 | 0.41 | 0 |
| t824937 | Library | 140447.8 | 12195.82 | 3271.16 | 247.67 | 0 | 0 | 97.72 | 2.28 | 0 |
| t825151 | Library | 141671.2 | 36473.09 | 1109.14 | 373.52 | 0 | 0 | 99.22 | 0.78 | 0 |
| t825708 | Library | 141893.5 | 27105.17 | 1911.75 | 841.34 | 7024.4 | 14048.8 | 94.08 | 1.27 | 4.66 |
| t825119 | Library | 142503.6 | 19647.71 | 1364.73 | 123.32 | 0 | 0 | 99.05 | 0.95 | 0 |
| t825009 | Library | 142816 | 31498.94 | 797.23 | 927.08 | 0 | 0 | 99.44 | 0.56 | 0 |
| t825109 | Library | 146421.8 | 17672.45 | 1426.59 | 159.08 | 0 | 0 | 99.04 | 0.96 | 0 |
| t825273 | Library | 148182.2 | 60701.09 | 1326.73 | 505.65 | 0 | 0 | 99.11 | 0.89 | 0 |
| t825023 | Library | 148242.5 | 31939.16 | 840.04 | 976.56 | 0 | 0 | 99.44 | 0.56 | 0 |
| t826237 | Library | 149070.3 | 5630.51 | 2401.17 | 136.07 | 0 | 0 | 98.41 | 1.59 | 0 |
| t824663 | Library | 149115.9 | 11650.5 | 941.6 | 1087.48 | 0 | 0 | 99.37 | 0.63 | 0 |
| t826137 | Library | 152044.3 | 14662.16 | 0 | 0 | 0 | 0 | 100 | 0 | 0 |
| t826100 | Library | 155826.3 | 23157.51 | 1352.42 | 91.33 | 0 | 0 | 99.14 | 0.86 | 0 |
| t825129 | Library | 157925 | 38488.72 | 1521.87 | 279.2 | 0 | 0 | 99.05 | 0.95 | 0 |
| t825297 | Library | 158813.5 | 38860.74 | 693.74 | 466.13 | 0 | 0 | 99.57 | 0.43 | 0 |
| t825057 | Library | 158892.3 | 11804.47 | 0 | 0 | 0 | 0 | 100 | 0 | 0 |
| t825029 | Library | 159426.9 | 2948.28 | 2388.92 | 126.02 | 0 | 0 | 98.52 | 1.48 | 0 |
| t825059 | Library | 160127.3 | 8557.53 | 777.36 | 897.62 | 0 | 0 | 99.52 | 0.48 | 0 |
| t824990 | Library | 161446.9 | 7696.97 | 1571.34 | 131.28 | 0 | 0 | 99.04 | 0.96 | 0 |
| t825013 | Library | 162119.3 | 6793.15 | 1570.23 | 110.25 | 0 | 0 | 99.04 | 0.96 | 0 |
| t825087 | Library | 162372.2 | 53136.98 | 0 | 0 | 0 | 0 | 100 | 0 | 0 |
| t825101 | Library | 163052.5 | 15634.97 | 454.32 | 534.85 | 0 | 0 | 99.72 | 0.28 | 0 |
| t825103 | Library | 164911.6 | 23105.15 | 631.73 | 1263.46 | 9694.81 | 19389.62 | 94.11 | 0.36 | 5.53 |
| t825049 | Library | 168132.5 | 15365.1 | 0 | 0 | 0 | 0 | 100 | 0 | 0 |
| t826280 | Library | 168823 | 20342.63 | 408.16 | 816.32 | 0 | 0 | 99.76 | 0.24 | 0 |
| t826278 | Library | 168941.8 | 36980.64 | 234.1 | 468.2 | 0 | 0 | 99.86 | 0.14 | 0 |
| t825621 | Library | 169273 | 36038.77 | 392.56 | 484.42 | 0 | 0 | 99.77 | 0.23 | 0 |
| t826208 | Library | 172607.3 | 50188.09 | 0 | 0 | 0 | 0 | 100 | 0 | 0 |
| t825086 | Library | 174306.5 | 27191.83 | 1910.66 | 420.29 | 0 | 0 | 98.92 | 1.08 | 0 |
| t825058 | Library | 177386.9 | 41557.34 | 546.93 | 373.06 | 0 | 0 | 99.69 | 0.31 | 0 |
| t826279 | Library | 180275.1 | 38783.05 | 1148.51 | 1139.49 | 0 | 0 | 99.37 | 0.63 | 0 |
| t825084 | Library | 184217.3 | 38703.58 | 199.21 | 398.42 | 0 | 0 | 99.89 | 0.11 | 0 |
| t826132 | Library | 184563.6 | 15285.63 | 1561.38 | 120.15 | 0 | 0 | 99.16 | 0.84 | 0 |
| t825263 | Library | 187383.3 | 60083.17 | 1603.21 | 1089.08 | 0 | 0 | 99.15 | 0.85 | 0 |
| t825221 | Library | 189500.8 | 29129.78 | 641.91 | 754.51 | 0 | 0 | 99.66 | 0.34 | 0 |
| t825099 | Library | 196013.3 | 34192.28 | 582.52 | 690.44 | 0 | 0 | 99.7 | 0.3 | 0 |
| t825085 | Library | 200770.4 | 24444.13 | 1377.25 | 179.39 | 0 | 0 | 99.32 | 0.68 | 0 |
| t825496 | Library | 203802.5 | 15014.44 | 2435.85 | 23.04 | 0 | 0 | 98.82 | 1.18 | 0 |
| t825914 | Library | 214120.7 | 38346.58 | 1259.72 | 845.31 | 0 | 0 | 99.42 | 0.58 | 0 |
| t826054 | Library | 229157.9 | 23616.43 | 1754.5 | 142.43 | 0 | 0 | 99.24 | 0.76 | 0 |

TABLE 15
Terminal C4 Product Titers of terminal synthase candidate enzymes in S. cerevisiae

| | | Mean | Std Dev | Mean | Std Dev | Mean | Std Dev | % | % | % |
|---|---|---|---|---|---|---|---|---|---|---|
| Strain | Strain | THCVA | THCVA | CBDVA | CBDVA | CBCVA | CBCVA | Product | Product | Product |
| ID | Type | [μg/L] | [μg/L] | [μg/L] | [μg/L] | [μg/L] | [μg/L] | THCVA | CBDVA | CBCVA |
| t820182 | Pos. Ctrl. | 14529.18 | 5730.92 | 0 | 0 | 62.84 | 266.62 | 99.57 | 0.00 | 0.43 |
| | (C. sativa | | | | | | | | | |
| | THCAS) | | | | | | | | | |
| t807973 | Pos. Ctrl. | 408.16 | 413.01 | 6187.43 | 3092.57 | 603.91 | 535.49 | 5.67 | 85.94 | 8.39 |
| | (C. sativa | | | | | | | | | |
| | CBDAS) | | | | | | | | | |
| t807949 | Pos. Ctrl | 34976.47 | 5350.18 | 0 | 0 | 0 | 0 | 100 | 0 | 0 |
| | (C. sativa | | | | | | | | | |
| | THCAS) | | | | | | | | | |
| t807914 | GFP | 0.00 | 0.00 | 0.00 | 0.00 | 0.00 | 0.00 | — | — | — |
| | negative | | | | | | | | | |
| | ctrl | | | | | | | | | |
| t826093 | Library | 4321.86 | 874.75 | 22201.28 | 2293.59 | 2702.18 | 275.39 | 14.79 | 75.97 | 9.25 |
| t826274 | Library | 5337.24 | 1789.65 | 16830.56 | 4397.14 | 2511.88 | 823.82 | 21.63 | 68.20 | 10.18 |
| t825987 | Library | 4434.11 | 258.03 | 15918.05 | 1009.78 | 2297.02 | 130.69 | 19.58 | 70.28 | 10.14 |
| t826072 | Library | 3240.22 | 308.53 | 19255.04 | 1594.67 | 2670.62 | 150.83 | 12.88 | 76.51 | 10.61 |
| t826096 | Library | 1486.61 | 62.35 | 9545.11 | 647.53 | 1369.79 | 103.39 | 11.99 | 76.97 | 11.05 |
| t825125 | Library | 352.02 | 408.81 | 7549.17 | 640.17 | 1159.41 | 191.17 | 3.89 | 83.32 | 12.80 |
| t825217 | Library | 896.00 | 99.61 | 8324.30 | 752.13 | 1358.31 | 182.96 | 8.47 | 78.69 | 12.84 |
| t825189 | Library | 665.84 | 72.27 | 6137.93 | 540.08 | 925.34 | 94.78 | 8.61 | 79.41 | 11.97 |
| t825341 | Library | 5629.23 | 628.81 | 27017.35 | 1851.79 | 5247.27 | 821.35 | 14.86 | 71.30 | 13.85 |
| t825248 | Library | 4371.65 | 505.92 | 23631.90 | 2696.59 | 2698.49 | 349.03 | 14.24 | 76.97 | 8.79 |
| t824773 | Library | 3506.09 | 835.78 | 19269.98 | 4532.02 | 2227.50 | 553.55 | 14.02 | 77.07 | 8.91 |
| t825766 | Library | 4432.91 | 198.33 | 22012.97 | 1246.86 | 2799.67 | 246.27 | 15.16 | 75.27 | 9.57 |
| t824918 | Library | 4571.46 | 333.10 | 15654.02 | 1056.73 | 2198.08 | 290.49 | 20.39 | 69.81 | 9.80 |
| t825034 | Library | 3964.61 | 70.35 | 24085.85 | 841.58 | 3249.53 | 90.91 | 12.67 | 76.95 | 10.38 |
| t824932 | Library | 3447.25 | 296.25 | 0.00 | 0.00 | 9301.63 | 954.05 | 27.04 | 0.00 | 72.96 |
| t824618 | Library | 2234.07 | 165.27 | 0.00 | 0.00 | 14836.06 | 1103.65 | 13.09 | 0.00 | 86.91 |
| t825126 | Library | 4436.97 | 363.60 | 25188.19 | 1480.74 | 3284.76 | 456.69 | 13.48 | 76.54 | 9.98 |
| t825910 | Library | 4249.71 | 584.70 | 0.00 | 0.00 | 8490.14 | 920.00 | 33.36 | 0.00 | 66.64 |
| t824688 | Library | 4228.74 | 231.67 | 20597.69 | 1231.62 | 2619.74 | 187.23 | 15.41 | 75.05 | 9.55 |
| t824996 | Library | 3855.66 | 537.82 | 0.00 | 0.00 | 9165.52 | 1200.54 | 29.61 | 0.00 | 70.39 |
| t825528 | Library | 3692.70 | 1227.13 | 0.00 | 0.00 | 11358.95 | 5291.80 | 24.53 | 0.00 | 75.47 |
| t825154 | Library | 3780.39 | 504.36 | 19468.96 | 1850.65 | 1891.65 | 81.10 | 15.04 | 77.44 | 7.52 |
| t825930 | Library | 3974.90 | 225.37 | 20267.81 | 2083.54 | 2280.36 | 226.71 | 14.99 | 76.42 | 8.60 |
| t825043 | Library | 3769.89 | 133.82 | 0.00 | 0.00 | 8797.72 | 165.86 | 30.00 | 0.00 | 70.00 |
| t824807 | Library | 3357.53 | 468.33 | 17960.57 | 2948.48 | 1955.35 | 304.15 | 14.43 | 77.17 | 8.40 |
| t825978 | Library | 3729.92 | 259.78 | 0.00 | 0.00 | 7803.92 | 372.79 | 32.34 | 0.00 | 67.66 |
| t825277 | Library | 5074.27 | 210.87 | 22201.95 | 1240.95 | 2353.52 | 143.70 | 17.13 | 74.93 | 7.94 |
| t824498 | Library | 4275.89 | 465.24 | 0.00 | 0.00 | 10427.31 | 1145.45 | 29.08 | 0.00 | 70.92 |
| t824603 | Library | 4379.66 | 1065.39 | 24174.72 | 4819.93 | 3318.99 | 605.21 | 13.74 | 75.85 | 10.41 |
| t826076 | Library | 2441.37 | 305.28 | 14634.39 | 1941.64 | 2008.75 | 203.43 | 12.79 | 76.68 | 10.53 |
| t825259 | Library | 3573.58 | 375.88 | 0.00 | 0.00 | 8531.40 | 591.48 | 29.52 | 0.00 | 70.48 |
| t825936 | Library | 4032.17 | 413.09 | 19537.02 | 1670.61 | 2194.60 | 102.06 | 15.65 | 75.83 | 8.52 |
| t825077 | Library | 2155.17 | 1730.32 | 0.00 | 0.00 | 2484.33 | 1708.76 | 46.45 | 0.00 | 53.55 |
| t825123 | Library | 7599.87 | 641.42 | 24191.10 | 2744.83 | 1412.40 | 270.25 | 22.89 | 72.86 | 4.25 |
| t825215 | Library | 9326.54 | 1215.67 | 26027.12 | 3050.69 | 1614.25 | 169.10 | 25.23 | 70.40 | 4.37 |
| t825585 | Library | 6037.88 | 556.91 | 21641.94 | 3482.09 | 1914.74 | 272.64 | 20.40 | 73.13 | 6.47 |
| t825012 | Library | 9497.41 | 988.11 | 19473.00 | 1861.66 | 1190.75 | 138.92 | 31.49 | 64.56 | 3.95 |
| t825773 | Library | 5019.73 | 5801.69 | 14028.58 | 16230.64 | 957.05 | 1127.09 | 25.09 | 70.12 | 4.78 |
| t824786 | Library | 9465.74 | 630.83 | 18332.26 | 823.19 | 1083.78 | 138.77 | 32.77 | 63.47 | 3.75 |
| t826099 | Library | 8536.43 | 1555.22 | 21164.31 | 3491.54 | 1111.91 | 279.26 | 27.70 | 68.69 | 3.61 |
| t824942 | Library | 9637.56 | 676.86 | 19367.38 | 593.59 | 1298.29 | 110.39 | 31.80 | 63.91 | 4.28 |
| t826070 | Library | 1838.10 | 126.64 | 11332.79 | 241.75 | 1578.81 | 159.43 | 12.46 | 76.83 | 10.70 |
| t824626 | Library | 9628.53 | 1087.07 | 19550.88 | 1922.42 | 1192.07 | 311.35 | 31.70 | 64.37 | 3.92 |
| t825269 | Library | 5195.17 | 1035.20 | 0.00 | 0.00 | 11747.76 | 1893.18 | 30.66 | 0.00 | 69.34 |
| t824654 | Library | 10432.93 | 261.81 | 21619.69 | 1050.85 | 1441.42 | 158.82 | 31.15 | 64.55 | 4.30 |
| t824746 | Library | 7720.20 | 2333.22 | 14717.67 | 4287.31 | 1053.73 | 339.36 | 32.86 | 62.65 | 4.49 |
| t825877 | Library | 9079.32 | 1219.31 | 25746.05 | 2523.07 | 1796.26 | 380.23 | 24.79 | 70.30 | 4.90 |
| t825841 | Library | 9197.24 | 1630.34 | 26974.43 | 4380.52 | 1777.75 | 490.26 | 24.24 | 71.08 | 4.68 |
| t824646 | Library | 9300.69 | 513.73 | 18821.50 | 596.45 | 1053.66 | 105.90 | 31.88 | 64.51 | 3.61 |
| t824659 | Library | 9748.50 | 680.11 | 22599.41 | 1678.38 | 1422.74 | 94.41 | 28.87 | 66.92 | 4.21 |
| t824540 | Library | 10207.46 | 895.68 | 19756.69 | 1652.76 | 1193.12 | 107.16 | 32.76 | 63.41 | 3.83 |
| t825933 | Library | 10623.28 | 958.55 | 19771.76 | 1412.32 | 1246.88 | 62.35 | 33.57 | 62.49 | 3.94 |
| t826097 | Library | 10415.16 | 2359.49 | 25830.80 | 5187.66 | 1676.74 | 558.04 | 27.46 | 68.11 | 4.42 |
| t824571 | Library | 10440.61 | 565.69 | 21058.96 | 1248.13 | 1343.30 | 135.18 | 31.79 | 64.12 | 4.09 |
| t825593 | Library | 11126.13 | 926.97 | 29681.63 | 1998.42 | 2115.17 | 298.99 | 25.92 | 69.15 | 4.93 |
| t825108 | Library | 7653.41 | 4744.76 | 28652.01 | 18264.79 | 0.00 | 0.00 | 21.08 | 78.92 | 0.00 |
| t825724 | Library | 7323.44 | 879.24 | 21225.76 | 2526.53 | 1057.24 | 266.61 | 24.74 | 71.69 | 3.57 |
| t824539 | Library | 9337.12 | 521.90 | 25355.87 | 1416.98 | 1666.36 | 72.96 | 25.68 | 69.74 | 4.58 |
| t824653 | Library | 8863.94 | 644.52 | 23029.83 | 1556.13 | 1317.44 | 110.41 | 26.69 | 69.34 | 3.97 |
| t825833 | Library | 8616.50 | 721.13 | 24316.58 | 2124.16 | 1890.45 | 116.27 | 24.74 | 69.83 | 5.43 |
| t825739 | Library | 7727.89 | 315.61 | 21730.09 | 527.57 | 1205.07 | 63.69 | 25.20 | 70.87 | 3.93 |
| t825907 | Library | 7929.94 | 274.23 | 19593.35 | 656.53 | 1007.51 | 58.18 | 27.79 | 68.67 | 3.53 |
| t824625 | Library | 8673.34 | 1029.55 | 23513.35 | 2629.04 | 1266.23 | 270.65 | 25.93 | 70.29 | 3.79 |
| t825889 | Library | 11115.46 | 1097.61 | 29414.81 | 4085.37 | 2189.43 | 647.47 | 26.02 | 68.86 | 5.13 |
| t824612 | Library | 9185.88 | 869.54 | 23932.43 | 2376.27 | 1385.84 | 167.59 | 26.62 | 69.36 | 4.02 |
| t825862 | Library | 14078.06 | 531.57 | 37241.92 | 4595.35 | 3141.00 | 783.77 | 25.85 | 68.38 | 5.77 |
| t825935 | Library | 8044.61 | 523.74 | 19961.33 | 1594.94 | 993.67 | 59.53 | 27.74 | 68.83 | 3.43 |
| t825105 | Library | 13147.81 | 15198.18 | 0.00 | 0.00 | 0.00 | 0.00 | 100.00 | 0.00 | 0.00 |
| t825301 | Library | 19582.25 | 22626.33 | 0.00 | 0.00 | 0.00 | 0.00 | 100.00 | 0.00 | 0.00 |
| t825377 | Library | 36474.30 | 2088.63 | 0.00 | 0.00 | 0.00 | 0.00 | 100.00 | 0.00 | 0.00 |
| t825092 | Library | 16862.83 | 19491.28 | 0.00 | 0.00 | 0.00 | 0.00 | 100.00 | 0.00 | 0.00 |
| t825075 | Library | 11794.34 | 16616.26 | 0.00 | 0.00 | 0.00 | 0.00 | 100.00 | 0.00 | 0.00 |
| t825213 | Library | 42405.35 | 3783.51 | 106.19 | 123.94 | 0.00 | 0.00 | 99.75 | 0.25 | 0.00 |
| t824771 | Library | 26382.43 | 1302.16 | 0.00 | 0.00 | 0.00 | 0.00 | 100.00 | 0.00 | 0.00 |
| t825025 | Library | 34244.09 | 2738.57 | 0.00 | 0.00 | 0.00 | 0.00 | 100.00 | 0.00 | 0.00 |
| t825090 | Library | 19213.63 | 22768.12 | 0.00 | 0.00 | 0.00 | 0.00 | 100.00 | 0.00 | 0.00 |
| t826284 | Library | 23696.59 | 7236.48 | 0.00 | 0.00 | 0.00 | 0.00 | 100.00 | 0.00 | 0.00 |
| t825024 | Library | 22920.29 | 3593.27 | 0.00 | 0.00 | 0.00 | 0.00 | 100.00 | 0.00 | 0.00 |
| t825015 | Library | 19634.76 | 14603.34 | 0.00 | 0.00 | 0.00 | 0.00 | 100.00 | 0.00 | 0.00 |
| t824526 | Library | 30875.70 | 1665.38 | 0.00 | 0.00 | 0.00 | 0.00 | 100.00 | 0.00 | 0.00 |
| t824647 | Library | 33029.61 | 1333.89 | 32.01 | 64.01 | 0.00 | 0.00 | 99.90 | 0.10 | 0.00 |
| t825007 | Library | 27135.4 | 5339.70 | 0.00 | 0.00 | 0.00 | 0.00 | 100.00 | 0.00 | 0.00 |
| t825501 | Library | 34961.69 | 3138.17 | 0.00 | 0.00 | 0.00 | 0.00 | 100.00 | 0.00 | 0.00 |
| t825219 | Library | 35322.67 | 1961.90 | 0.00 | 0.00 | 0.00 | 0.00 | 100.00 | 0.00 | 0.00 |
| t825286 | Library | 34322.20 | 7206.21 | 0.00 | 0.00 | 0.00 | 0.00 | 100.00 | 0.00 | 0.00 |
| t825071 | Library | 25250.80 | 5267.38 | 0.00 | 0.00 | 0.00 | 0.00 | 100.00 | 0.00 | 0.00 |
| t825047 | Library | 29104.28 | 693.89 | 0.00 | 0.00 | 0.00 | 0.00 | 100.00 | 0.00 | 0.00 |
| t825031 | Library | 28420.36 | 4191.07 | 0.00 | 0.00 | 0.00 | 0.00 | 100.00 | 0.00 | 0.00 |
| t825633 | Library | 25500.80 | 1946.60 | 50.30 | 100.59 | 0.00 | 0.00 | 99.80 | 0.20 | 0.00 |
| t824861 | Library | 28384.900 | 1197.54 | 0.00 | 0.00 | 0.00 | 0.00 | 100.00 | 0.00 | 0.00 |
| t824928 | Library | 30530.55 | 2056.23 | 219.07 | 42.26 | 0.00 | 0.00 | 99.29 | 0.71 | 0.00 |
| t825725 | Library | 29979.43 | 3470.58 | 0.00 | 0.00 | 423.01 | 846.01 | 98.61 | 0.00 | 1.39 |
| t826036 | Library | 26987.28 | 2239.20 | 0.00 | 0.00 | 0.00 | 0.00 | 100.00 | 0.00 | 0.00 |
| t824903 | Library | 28246.56 | 2612.35 | 32.84 | 65.68 | 0.00 | 0.00 | 99.88 | 0.12 | 0.00 |
| t824845 | Library | 23246.37 | 6435.16 | 0.00 | 0.00 | 0.00 | 0.00 | 100.00 | 0.00 | 0.00 |
| t825078 | Library | 24598.30 | 8324.67 | 160.67 | 210.74 | 395.87 | 791.74 | 97.79 | 0.64 | 1.57 |
| t826030 | Library | 28584.94 | 4128.51 | 0.00 | 0.00 | 0.00 | 0.00 | 100.00 | 0.00 | 0.00 |
| t824622 | Library | 34261.87 | 3389.90 | 35.28 | 70.56 | 0.00 | 0.00 | 99.90 | 0.10 | 0.00 |
| t825141 | Library | 27482.09 | 4075.99 | 0.00 | 0.00 | 0.00 | 0.00 | 100.00 | 0.00 | 0.00 |
| t825379 | Library | 48037.63 | 6303.86 | 177.77 | 140.94 | 730.79 | 1461.57 | 98.14 | 0.36 | 1.49 |
| t825625 | Library | 32685.23 | 2612.03 | 0.00 | 0.00 | 0.00 | 0.00 | 100.00 | 0.00 | 0.00 |
| t825309 | Library | 34810.24 | 4108.46 | 36.73 | 73.46 | 0.00 | 0.00 | 99.89 | 0.11 | 0.00 |
| t824930 | Library | 25828.33 | 604.78 | 0.00 | 0.00 | 0.00 | 0.00 | 100.00 | 0.00 | 0.00 |
| t824937 | Library | 27567.91 | 1462.49 | 215.12 | 7.12 | 0.00 | 0.00 | 99.23 | 0.77 | 0.00 |
| t825151 | Library | 37767.14 | 4072.38 | 47.87 | 95.73 | 0.00 | 0.00 | 99.87 | 0.13 | 0.00 |
| t825708 | Library | 27634.38 | 1409.03 | 0.00 | 0.00 | 0.00 | 0.00 | 100.00 | 0.00 | 0.00 |
| t825119 | Library | 26504.59 | 8311.25 | 0.00 | 0.00 | 0.00 | 0.00 | 100.00 | 0.00 | 0.00 |
| t825009 | Library | 27036.30 | 7746.65 | 0.00 | 0.00 | 0.00 | 0.00 | 100.00 | 0.00 | 0.00 |
| t825109 | Library | 33149.60 | 6509.72 | 0.00 | 0.00 | 0.00 | 0.00 | 100.00 | 0.00 | 0.00 |
| t825273 | Library | 29577.96 | 1023.05 | 32.29 | 64.58 | 0.00 | 0.00 | 99.89 | 0.11 | 0.00 |
| t825023 | Library | 31656.21 | 949.42 | 0.00 | 0.00 | 0.00 | 0.00 | 100.00 | 0.00 | 0.00 |
| t826237 | Library | 27902.45 | 1341.04 | 0.00 | 0.00 | 0.00 | 0.00 | 100.00 | 0.00 | 0.00 |
| t824663 | Library | 26987.01 | 5838.71 | 36.59 | 73.17 | 0.00 | 0.00 | 99.86 | 0.14 | 0.00 |
| t826137 | Library | 24139.22 | 4414.79 | 0.00 | 0.00 | 0.00 | 0.00 | 100.00 | 0.00 | 0.00 |
| t826100 | Library | 36962.29 | 1755.86 | 108.33 | 131.42 | 669.79 | 773.48 | 97.94 | 0.29 | 1.77 |
| t825129 | Library | 38487.37 | 6157.27 | 82.36 | 95.57 | 0.00 | 0.00 | 99.79 | 0.21 | 0.00 |
| t825297 | Library | 37859.38 | 2886.17 | 38.69 | 77.38 | 0.00 | 0.00 | 99.90 | 0.10 | 0.00 |
| t825057 | Library | 23954.88 | 16177.90 | 0.00 | 0.00 | 0.00 | 0.00 | 100.00 | 0.00 | 0.00 |
| t825029 | Library | 31780.71 | 1429.73 | 0.00 | 0.00 | 0.00 | 0.00 | 100.00 | 0.00 | 0.00 |
| t825059 | Library | 26880.58 | 10409.12 | 0.00 | 0.00 | 0.00 | 0.00 | 100.00 | 0.00 | 0.00 |
| t824990 | Library | 37070.20 | 1503.42 | 0.00 | 0.00 | 0.00 | 0.00 | 100.00 | 0.00 | 0.00 |
| t825013 | Library | 32494.40 | 2849.71 | 0.00 | 0.00 | 0.00 | 0.00 | 100.00 | 0.00 | 0.00 |
| t825087 | Library | 27234.02 | 8671.51 | 0.00 | 0.00 | 0.00 | 0.00 | 100.00 | 0.00 | 0.00 |
| t825101 | Library | 23648.49 | 2058.43 | 0.00 | 0.00 | 0.00 | 0.00 | 100.00 | 0.00 | 0.00 |
| t825103 | Library | 24958.25 | 18727.48 | 0.00 | 0.00 | 0.00 | 0.00 | 100.00 | 0.00 | 0.00 |
| t825049 | Library | 43708.91 | 4062.54 | 0.00 | 0.00 | 0.00 | 0.00 | 100.00 | 0.00 | 0.00 |
| t826280 | Library | 33920.91 | 5572.79 | 21.71 | 43.41 | 507.42 | 1014.85 | 98.46 | 0.06 | 1.47 |
| t826278 | Library | 31879.47 | 9925.12 | 0.00 | 0.00 | 0.00 | 0.00 | 100.00 | 0.00 | 0.00 |
| t825621 | Library | 34723.77 | 3274.99 | 0.00 | 0.00 | 0.00 | 0.00 | 100.00 | 0.00 | 0.00 |
| t826208 | Library | 18457.17 | 12780.64 | 0.00 | 0.00 | 0.00 | 0.00 | 100.00 | 0.00 | 0.00 |
| t825086 | Library | 43598.62 | 5155.48 | 0.00 | 0.00 | 0.00 | 0.00 | 100.00 | 0.00 | 0.00 |
| t825058 | Library | 48071.51 | 8507.37 | 0.00 | 0.00 | 0.00 | 0.00 | 100.00 | 0.00 | 0.00 |
| t826279 | Library | 39910.99 | 1936.63 | 0.00 | 0.00 | 429.32 | 858.63 | 98.94 | 0.00 | 1.06 |
| t825084 | Library | 32568.21 | 14809.65 | 0.00 | 0.00 | 0.00 | 0.00 | 100.00 | 0.00 | 0.00 |
| t826132 | Library | 35281.0 | 4178.20 | 37.75 | 75.50 | 0.00 | 0.00 | 99.89 | 0.11 | 0.00 |
| t825263 | Library | 26069.15 | 6019.54 | 0.00 | 0.00 | 0.00 | 0.00 | 100.00 | 0.00 | 0.00 |
| t825221 | Library | 33289.84 | 2352.64 | 40.50 | 81.00 | 0.00 | 0.00 | 99.88 | 0.12 | 0.00 |
| t825099 | Library | 32649.38 | 1539.67 | 0.00 | 0.00 | 0.00 | 0.00 | 100.00 | 0.00 | 0.00 |
| t825085 | Library | 42734.57 | 9999.19 | 0.00 | 0.00 | 0.00 | 0.00 | 100.00 | 0.00 | 0.00 |
| t825496 | Library | 33924.06 | 1916.82 | 0.00 | 0.00 | 0.00 | 0.00 | 100.00 | 0.00 | 0.00 |
| t825914 | Library | 25327.08 | 1447.87 | 0.00 | 0.00 | 0.00 | 0.00 | 100.00 | 0.00 | 0.00 |
| t826054 | Library | 28715.87 | 1053.97 | 0.00 | 0.00 | 0.00 | 0.00 | 100.00 | 0.00 | 0.00 |

### Example 5: Screen to Improve Functional Expression of Additional Terminal Synthases

To further improve functional expression of TSs identified in the Examples presented above, a library of approximately 1324 candidate TSs was designed using three different strategies: (1) recombination of single mutations from Example 3; (2) recombination of mutations enriched in top designs from Example 4; and (3) structure informed single mutations.

(1) Recombination of single mutations: Variant abundance data were derived from a site-scanning library on candidate Terminal Synthases from Example 3. The variant abundance was determined in a multiplexed assay wherein synthetic TS polypeptides were expressed as genetic fusions to Aga2 on the cell surface. The per-cell abundance of the TS-Aga2 fusions were determined by labeling with fluorescently conjugated antibodies specific for a terminal Myc epitope. Cells were isolated based on this fluorescence at a single-cell level. Variants that were brighter were assumed to be able to be expressed at a high level, due to some combination of increased thermal and colloidal stability. The relative brightnesses were quantified and summarized as a final computed enrichment score. Additional single mutations were derived from a position-specific scoring matrix which scores amino acids at each position as either being present more or less often within a larger multiple sequence alignment than one would expect by chance. Finally, mutations harbored by single mutant TS candidates from Example 3 that were at least 2-fold more active than their control were also used. Mutations from these three sources were recombined to produce a total of 365 protein sequences.

(2) Recombination of top designs from Example 4: Several engineered TS candidates from Example 4 demonstrated significant improvements in activity relative to wild type controls. The mutations harbored by the top quartile of TS candidates, based upon THCA titer, were recombined to find the best combinations of mutants. The mutations harbored by the top quartile of TS candidates, based upon CBDA titer, were recombined to generate all possible combinations. Mutations from this design strategy produced a total of 363 protein sequences.

(3) Structure informed single mutations: C. sativa THCAS and CBDAS are structurally similar enzymes which share ˜85% sequence identity and differentially cyclize the same substrate, CBGA, to yield their respective products. Whether CBGA is converted to THCA or CBDA is speculated to depend on the target of a nucleophilic attack by a catalytic base within the active pocket of the terminal synthase enzyme (Shoyama et al. (2012) JMB 423(1):96-105 and Taura et al. (2007) FEBS Letters 581(16):2929-2934). In the case of THCA formation, the catalytic base is believed to be facilitated by Y484 which deprotonates O6′ of CBGA. In the case of CBDA formation, the catalytic base is less well characterized but structural and sequence similarities with THCAS suggest that it may be Y483. Mutations within the presumed inner shell of CBDAS (e.g., <8 Å from the catalytic residues) and within the presumed outer shell of the THCAS (e.g., >30 Å from the catalytic residues) were generated. Mutations from this design strategy produced a total of 573 protein sequences.

The TS candidate genes were recoded in silico for expression in S. cerevisiae and synthesized in the integrative yeast expression vector shown in FIG. 5. Each candidate enzyme expression construct was transformed into a S. cerevisiae CEN.PK strain that also expressed a prenyltransferase enzyme capable of catalyzing reaction R4 in FIG. 2. Strain 865977, expressing a THCAS candidate from Example 4 (corresponding to strain t826279 in Example 4), was included in the library screen as a positive control for THCAS activity. Strain 865859, expressing a CBDAS candidate from Example 4 (corresponding to strain t824625 in Example 4), was included in the library screen as a positive control for CBDAS activity. Strains 876606 and 876607 expressing C. sativa THCAS (Uniprot Accession: I1V0C5) and C. saliva CBDAS (Uniprot Accession ID: A6P6V9) were included as positive controls, but were not used to establish hit ranking. All candidate enzymes in the library, and positive controls, were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

A terminal product assay was conducted as described in Example 4.

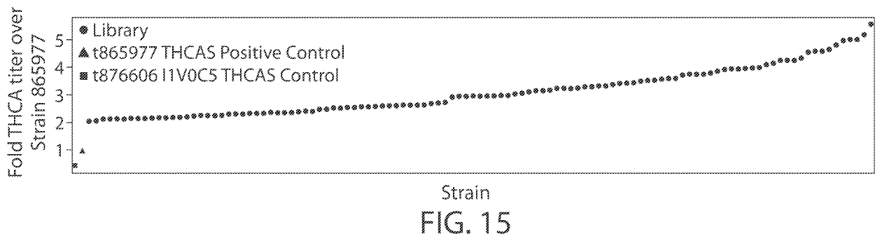

Strains engineered to produce THCA were normalized to the in-plate performance of strain 865977. 113 candidate TSs demonstrated a normalized THCA titer more than 2-fold greater than strain 865977 and over 4-fold greater than the wild type C. sativa THCAS harbored by strain 876606 (FIG. 15, Tables 16A-16B). 16 strains demonstrated THCA titers more than 4-fold greater than strain 865977 (FIG. 15, Tables 16A-16B), including: strain 924468, which expresses a TS that includes amino acid substitutions R31Q, A47T, H56N, Q58S, M61S, I74T, N90V, H143E, A250P, S255V, V288L, T340E, F345L, E424D, Q475K, and T492N relative to SEQ ID NO: 14; strain 924725, which expresses a TS that includes amino acid substitutions R31Q, H56N, Q58S, M61S, I74T, N90V, H143E, A250P, S255V, V288L, T340E, F345L, E424D, Q475K, and T492N relative to SEQ ID NO: 14; strain 924717, which expresses a TS that includes amino acid substitutions R31Q, A47T, V52L, H56N, Q58S, M61S, I74T, N90V, H143E, A250P, S255V, V288L, T340E, F345L, Q475K, and T492N relative to SEQ ID NO: 14; strain 924387, which expresses a TS that includes amino acid substitutions A47T, H56N, Q58S, M61S, I74T, N90V, A250D, S255V, F345L, Q475K, and T492N relative to SEQ ID NO: 14; strain 924472, which expresses a TS that includes amino acid substitutions R31Q, A47T, H56N, Q58S, M61S, I74T, N90V, H143E, A250P, S255V, V288L, T340E, F345L, Q475K, and T492N relative to SEQ ID NO: 14: strain 924487, which expresses a TS that includes amino acid substitutions R31Q, A47T, H56N, Q58S, M61S, I74T, N90V, A250P, S255V, T340E, F345L, E424D, Q475K, and T492N relative to SEQ ID NO: 14; strain 923862, which expresses a TS that includes amino acid substitutions R31Q, A47T, H56N, Q58S, M61S, I74T, N90V, H143E, A250P, S255V, T340E, F345L, Q475K, and T492N relative to SEQ ID NO: 14; strain 923908, which expresses a TS that includes amino acid substitutions R31Q, V52L, H56N, Q58S, M61S, I74T, N90V, A250P, S255V, V288L, F345L, Q475K, and T492N relative to SEQ ID NO: 14, strain 924276, which expresses a TS that includes amino acid substitutions A47T, H56N, Q58S, M61S, I74T, N90V, A250D, S255V, F345L, E424D, Q475K, and T492N relative to SEQ ID NO: 14; strain 924433, which expresses a TS that includes amino acid substitutions R31Q, H56N, Q58S, M61S, I74T, N90V, A250P, S255V, V288L, F345L, E424D, Q475K, and T492N relative to SEQ ID NO: 14; strain 923880, which expresses a TS that includes amino acid substitutions H56N, Q58S, M61S, I74T, N90V, H143E, A250D, S255V, V288L, F345L, Q475K, T492N, and A495E relative to SEQ ID NO: 14, strain 923609, which expresses a TS that includes amino acid substitutions H56N, Q58S, M61S, I74T, N90V, H143E, A250D, S255V, V288L, F345L, Q475K, and T492N relative to SEQ ID NO: 14; strain 924428, which expresses a TS that includes amino acid substitutions R31Q, A47T, H56N, Q58S, M61S, I74T, N90V, A250P, S255V, V288L, T340E, F345L, Q475K, and T492N relative to SEQ ID NO: 14; strain 924364, which expresses a TS that includes amino acid substitutions R31Q, H56N, Q58S, M61S, I74T, N90V, H143E, A250P, S255V, V288L, Q475K, and T492N relative to SEQ ID NO: 14; strain 924555, which expresses a TS that includes amino acid substitutions A47T, H56N, Q58S, M61S, I74T, N90V, A250D, S255V, V288L, F345L, Q475K, and T492N relative to SEQ ID NO: 14; and strain 924214, which expresses a TS that includes amino acid substitutions R31Q, V52I, H56N, M61S, I74T, N90V, A250P, S255V, F345L, Q475K, and T492N, relative to SEQ ID NO: 14.

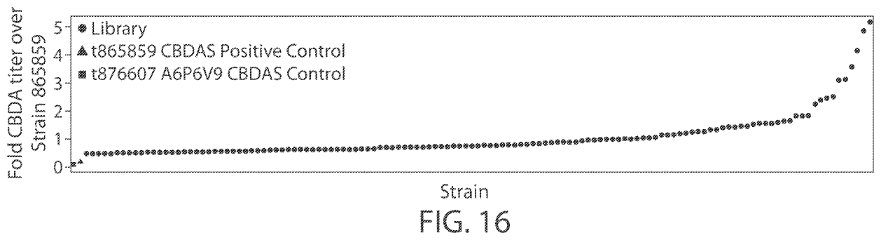

Likewise, strains engineered to produce CBDA were normalized to the in-plate performance of strain 865859. 128 candidate terminal synthases demonstrated a normalized CBDA titers more than 0.5-fold greater than strain 865859 and over 2-fold greater than the wild type C. sativa CBDAS harbored by strain 876607 (FIG. 16, Tables 16A-16B). 10 candidate terminal synthases demonstrated a normalized CBDA titers more than 2-fold greater than strain 865859 (FIG. 16, Tables 16A-16B), including: strain 924940, which expresses a TS that includes amino acid substitutions K50N, G95A, N196K, H213N, T339E, Q343E, L344M, and A414V, relative to SEQ ID NO: 13; strain 924748, which expresses a TS that includes amino acid substitutions G95A, Y175F, T339E, Q343E, and A414V relative to SEQ ID NO: 13; strain 924744, which expresses a TS that includes amino acid substitutions G95A, S116A, T339E, Q343E, A414V, and N527D relative to SEQ ID NO: 13; strain 924928, which expresses a TS that includes amino acid substitutions G95A, E150Q, V162I, C180G, N196K, N211D, N273H, T339E, Q343E, and A414V relative to SEQ ID NO: 13; strain 924747, which expresses a TS that includes amino acid substitutions G95A, T339E, Q343E, Q376V, and A414V relative to SEQ ID NO: 13; strain 924765, which expresses a TS that includes amino acid substitutions K50N, G95A, S100A, E150Q, V162I, C180G, N196K, N211D, H213N, S322E, T339E, Q343E, L344M, A414V, E452T, and I504Q relative to SEQ ID NO: 13; strain 923811, which expresses a TS that includes amino acid substitutions G95A, N196K, T339E, Q343E, and A414V relative to SEQ ID NO: 13; strain 924716, which expresses a TS that includes amino acid substitutions 50N, G95A, V103H, H213N, T339E, Q343E, L344M, and A414V relative to SEQ ID NO: 13, strain 924764, which expresses a TS that includes amino acid substitutions G95A, T339E, Q343E, Q376R, and A414V relative to SEQ ID NO: 13; and strain 924957, which expresses a TS that includes amino acid substitutions K50N, H213N, L230I, T339E, Q343E, and L344M relative to SEQ ID NO: 13.

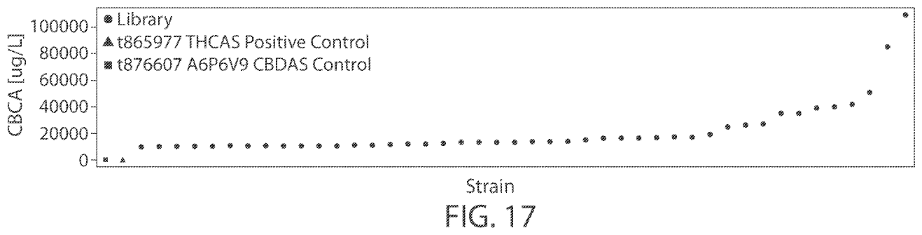

Surprisingly, 44 candidate TSs produced greater than 10 mg/L CBCA (FIG. 17, Table 16A). 19 strains demonstrated mean CBCA titers greater than 15 mg/L CBCA (FIG. 17, Table 16A), including: strain 923976, which expresses a TS that includes amino acid substitutions R31Q, H56N, Q58S, I74T, N90V, A250P, S255V, V288L, F345L, Q475K, and T492N relative to SEQ ID NO: 14; strain 923759 which expresses a TS that includes amino acid substitutions R31Q, V52I, H56N, Q58S, M61S, 174T, N90V, A250P, S255V, F345L, Q475K, and T492N relative to SEQ ID NO: 14; strain 923624, which expresses a TS that includes amino acid substitutions R31Q, H56N, I74T, N90V, H143E, A250P, S255V, Q475K, and T492N relative to SEQ ID NO: 14; strain 923980, which expresses a TS that includes amino acid substitutions R31Q, H56N, I74T, N90V, A250P, S255V, L443L, Q475K, and T492N relative to SEQ ID NO: 14; strain 923922, which expresses a TS that includes amino acid substitutions H56N, M61S, N90V, A250D, S255V, V288L, Q475K, T492N, and A495E relative to SEQ ID NO: 14; strain 923890, which expresses a TS that includes amino acid substitutions R31Q, H56N, I74T, N90V, K215R, A250P, S255V, Q475K, and T492N relative to SEQ ID NO: 14; strain 923616, which expresses a TS that includes amino acid substitutions R31Q, P49A, H56N, Q58S, M61S, I74T, N90V, H143E, A250P, S255V, V288L, F345L, Q475K, and T492N relative to SEQ ID NO: 14; strain 923954, which expresses a TS that includes amino acid substitutions R31Q, A47T, H56N, 174T, N90V, A250P, S255V, Q475K, and T492N relative to SEQ ID NO: 14; strain 923894, which expresses a TS that includes amino acid substitutions M61S, N90V, A250D, S255V, Q475K, T492N, A495E, and N498T relative to SEQ ID NO: 14; strain 923972, which expresses a TS that includes amino acid substitutions R31Q, H56N, M61S, I74T, N89H, N90V, S100A, H136R, E150Q, N196K, N21 ID, A250P, S255V, V288M, F345M, S382K, L443I, Q475K, and T492N relative to SEQ ID NO: 14, strain 923680, which expresses a TS that includes amino acid substitutions R31Q, H56N, Q58S, M61S, I74T, N90V, H143E, A250P, S255V, V288L, T340E, F345L, E424D, Q475K, and T492N relative to SEQ ID NO: 14; strain 923918, which expresses a TS that includes amino acid substitutions R31Q, H56N, I74T, S88L, N90V, A250P, S255V, Q475K, and T492N relative to SEQ ID NO: 14; strain 924465, which expresses a TS that includes amino acid substitutions R31Q, V52I, H56N, Q58S, M61S, I74T, N90V, H143E, A250P, S255V, V288L, F345L, Q475K, and T492N relative to SEQ ID NO: 14; strain 924725, which expresses a TS that includes amino acid substitutions R31Q, H56N, Q58S, M61S, I74T, N90V, H143E, A250P, S255V, V288L, T340E, F345L, E424D, Q475K, and T492N relative to SEQ ID NO: 14; strain 924927, which expresses a TS that includes amino acid substitutions R31Q, H56N, Q58S, M61S, I74T, N90V, H143E, A250P, S255V, V288L, F345L, A411V, Q475K, and T492N relative to SEQ ID NO: 14; strain 923908, which expresses a TS that includes amino acid substitutions R31Q, V52I, H56N, Q58S, M61S, 174T, N90V, A250P, S255V, V288L, F345L, Q475K, and T492N relative to SEQ ID NO: 14; strain 923613, which expresses a TS that includes amino acid substitutions R31Q, K50L, H56N, 174T, N90V, A250P, S255V, Q475K, and T492N relative to SEQ ID NO: 14; strain 924717, which expresses a TS that includes amino acid substitutions R311Q, A47T, V52I, H56N, Q58S, M61S, I74T, N90V, H143E, A250P, S255V, V288L, T340E, F345L, Q475K, and T492N relative to SEQ ID NO: 14; and strain 924309, which expresses a TS that includes amino acid substitutions R3Q, H56N, M61 S, I74T, N89K, N90V, S100A, N196K, N21 ID, A250P, S255V, I257R, V288K, F345M, S382K, L443I, Q475K, and T492N relative to SEQ ID NO: 14.

These putative CBCA synthases sample a mutagenic space different from the CBCASs discovered in Example 4. Of note are the differences in the terminal cannabinoid profile among the CBCASs discovered in Example 4 versus Example 5. CBCAS candidates in Example 4 range in their production of CBCA as a percentage of all terminal cannabinoids (Percent Product CBCA) from 41%-100% (Table 16B). The CBCAS candidates identified in Example 5 have a much lower Percent Product CBCA ceiling of ˜41% with most of the remaining terminal cannabinoid being THCA.

TABLE 16A
Terminal C6 Product Titers of terminal synthase candidate enzymes in S. cerevisiae

| | | Mean | Std. Dev. | Mean | Std. Dev. | Mean | Std. Dev. |
|---|---|---|---|---|---|---|---|
| Strain | Strain | THCA | THCA | CBDA | CBDA | CBCA | CBCA |
| ID | type | (ug/L) | (ug/L) | (ug/L) | (ug/L) | (ug/L) | (ug/L) |
| 865859 | Positive | 10960.03 | 3961.90 | 54379.21 | 17362.47 | 1119.84 | 1169.95 |
| | Control | | | | | | |
| | CBDAS; | | | | | | |
| | based on | | | | | | |
| | strain | | | | | | |
| | t824625 | | | | | | |
| 865977 | Positive | 69335.57 | 25212.66 | 296.53 | 452.42 | 1712.90 | 6412.00 |
| | Control | | | | | | |
| | THCAS; | | | | | | |
| | based on | | | | | | |
| | strain | | | | | | |
| | t826279 | | | | | | |
| 876606 | Positive | 27869.52 | 8869.33 | 31.59 | 110.72 | 292.40 | 1899.82 |
| | Control C. | | | | | | |
| | sativa | | | | | | |
| | THCAS; | | | | | | |
| | based on | | | | | | |
| | UniProt | | | | | | |
| | I1V0C5 | | | | | | |
| 876607 | Positive | 71.69 | 326.35 | 13918.31 | 5245.82 | 0.00 | 0.00 |
| | Control C. | | | | | | |
| | sativa | | | | | | |
| | CBDAS; | | | | | | |
| | based on | | | | | | |
| | UniProt | | | | | | |
| | A6P6V9 | | | | | | |
| 923976 | Library | 156121.76 | 12265.89 | 2559.20 | 286.41 | 109062.59 | 8376.65 |
| 923759 | Library | 246025.73 | 2402.28 | 2154.87 | 111.39 | 85403.83 | 120779.26 |
| 923624 | Library | 74450.99 | 25925.53 | 0.00 | 0.00 | 51011.97 | 16627.75 |
| 923980 | Library | 121465.20 | 3173.64 | 0.00 | 0.00 | 42136.81 | 59590.44 |
| 923922 | Library | 100765.19 | 5633.88 | 0.00 | 0.00 | 40096.22 | 49976.29 |
| 923890 | Library | 91086.74 | 21315.68 | 0.00 | 0.00 | 39060.50 | 51371.56 |
| 923616 | Library | 92067.89 | 11779.85 | 777.05 | 1098.92 | 35149.78 | 49709.29 |
| 923954 | Library | 91607.09 | 13725.96 | 0.00 | 0.00 | 35076.40 | 49605.51 |
| 923894 | Library | 38860.32 | 10099.45 | 0.00 | 0.00 | 27104.40 | 7102.31 |
| 923972 | Library | 208221.97 | 5504.13 | 6632.23 | 9379.39 | 26121.64 | 1828.41 |
| 923680 | Library | 53651.56 | 24133.34 | 801.09 | 1132.92 | 24905.01 | 35221.00 |
| 923918 | Library | 50798.54 | 4431.08 | 0.00 | 0.00 | 19067.12 | 26964.97 |
| 924465 | Library | 323258.56 | 26945.63 | 8745.60 | 2312.97 | 17412.81 | 3551.62 |
| 924725 | Library | 302090.05 | 14758.62 | 0.00 | 0.00 | 17196.57 | 933.03 |
| 924927 | Library | 339221.99 | 206303.90 | 0.00 | 0.00 | 16961.29 | 12391.95 |
| 923908 | Library | 346728.33 | 29157.39 | 11394.56 | 1416.99 | 16623.43 | 2075.56 |
| 923613 | Library | 45458.31 | 2777.77 | 0.00 | 0.00 | 16495.90 | 23328.73 |
| 924717 | Library | 292410.73 | 13874.18 | 0.00 | 0.00 | 16352.13 | 1070.95 |
| 924309 | Library | 125449.34 | 26809.34 | 10414.57 | 2109.80 | 15165.28 | 3338.02 |
| 923795 | Library | 38761.72 | 3212.69 | 0.00 | 0.00 | 14370.29 | 20322.66 |
| 923880 | Library | 327559.34 | 27290.70 | 6964.48 | 1380.10 | 14003.99 | 66.43 |
| 924364 | Library | 247423.14 | 11335.68 | 0.00 | 0.00 | 13670.82 | 1628.64 |
| 924612 | Library | 167155.67 | 4014.44 | 2298.50 | 35.92 | 13387.46 | 1053.61 |
| 924164 | Library | 124711.67 | 22662.76 | 10312.50 | 1531.43 | 13357.87 | 1950.04 |
| 924803 | Library | 119942.36 | 12957.62 | 990.59 | 1400.91 | 13343.29 | 1559.62 |
| 924559 | Library | 258202.68 | 138648.49 | 4792.07 | 822.39 | 13332.48 | 6613.94 |
| 924468 | Library | 289705.26 | 1444.03 | 12424.30 | 1703.18 | 12697.91 | 664.15 |
| 923884 | Library | 299245.18 | 17516.02 | 9892.51 | 282.51 | 12286.15 | 567.50 |
| 924276 | Library | 305035.31 | 20778.60 | 6199.77 | 151.56 | 11894.07 | 769.56 |
| 924665 | Library | 220936.32 | 64.50 | 0.00 | 0.00 | 11643.67 | 227.30 |
| 924691 | Library | 100660.20 | 134333.96 | 7818.24 | 11056.66 | 11104.60 | 15704.28 |
| 924639 | Library | 213286.80 | 97209.63 | 0.00 | 0.00 | 11055.19 | 5728.00 |
| 924512 | Library | 205682.79 | 10942.27 | 0.00 | 0.00 | 10958.14 | 561.66 |
| 924723 | Library | 205435.71 | 73246.40 | 0.00 | 0.00 | 10847.24 | 3628.06 |
| 923916 | Library | 245176.11 | 56508.17 | 6353.58 | 331.86 | 10753.81 | 1654.16 |
| 924428 | Library | 292915.18 | 46930.71 | 8451.23 | 334.93 | 10751.30 | 960.93 |
| 924472 | Library | 258707.40 | 20598.47 | 11099.61 | 139.41 | 10718.63 | 904.88 |
| 924609 | Library | 229160.52 | 86663.44 | 6622.19 | 1988.65 | 10580.20 | 4220.16 |
| 924657 | Library | 167982.82 | 11013.84 | 4565.30 | 65.97 | 10577.94 | 1522.02 |
| 924226 | Library | 271920.73 | 8419.39 | 5764.58 | 195.52 | 10566.22 | 1408.69 |
| 924306 | Library | 228730.09 | 3601.22 | 5203.83 | 87.56 | 10376.79 | 178.82 |
| 924695 | Library | 188428.21 | 14909.77 | 1570.26 | 5.30 | 10367.04 | 1012.67 |
| 924162 | Library | 226940.48 | 41720.64 | 7062.23 | 658.80 | 10225.71 | 3305.83 |
| 924387 | Library | 291971.64 | 8022.80 | 2382.16 | 120.83 | 10120.14 | 7.83 |
| 924667 | Library | 204155.15 | 21481.18 | 833.72 | 1179.06 | 9937.41 | 109.38 |
| 924425 | Library | 247763.95 | 63650.93 | 2283.06 | 563.13 | 9861.03 | 2744.76 |
| 924373 | Library | 191906.35 | 38935.19 | 1600.93 | 414.33 | 9830.90 | 1996.65 |
| 924212 | Library | 208865.32 | 69097.39 | 7242.75 | 1384.86 | 9807.60 | 4564.90 |
| 924635 | Library | 188844.02 | 6440.26 | 754.95 | 1067.66 | 9677.64 | 498.24 |
| 923912 | Library | 259314.75 | 22186.21 | 6958.28 | 54.90 | 9630.34 | 283.93 |
| 924304 | Library | 174922.40 | 243443.38 | 5739.65 | 8117.09 | 9553.59 | 13510.82 |
| 924028 | Library | 220273.46 | 191081.98 | 5823.00 | 5044.07 | 9433.43 | 8435.23 |
| 924426 | Library | 214079.45 | 185427.99 | 7728.56 | 6693.44 | 9245.77 | 8007.07 |
| 924160 | Library | 232558.30 | 2846.86 | 4849.52 | 281.81 | 9213.45 | 704.58 |
| 924424 | Library | 261208.24 | 19462.00 | 4507.90 | 129.95 | 9159.50 | 357.16 |
| 924431 | Library | 193529.35 | 37851.70 | 1821.91 | 421.69 | 8749.91 | 3139.18 |
| 924518 | Library | 216777.27 | 7567.35 | 2090.68 | 19.27 | 8632.60 | 183.69 |
| 923771 | Library | 323899.31 | 15568.39 | 1530.93 | 45.24 | 8573.42 | 804.94 |
| 923744 | Library | 276525.30 | 53975.12 | 5521.55 | 7808.65 | 8565.97 | 12114.11 |
| 924673 | Library | 144011.90 | 10356.74 | 0.00 | 0.00 | 8510.89 | 511.60 |
| 924398 | Library | 186606.55 | 163118.80 | 5348.47 | 4655.40 | 8501.03 | 7502.16 |
| 923948 | Library | 191135.66 | 23926.72 | 6174.72 | 516.68 | 8088.62 | 251.05 |
| 924062 | Library | 211238.45 | 48282.27 | 3873.45 | 409.00 | 7879.27 | 1531.65 |
| 923854 | Library | 162453.92 | 21857.68 | 5658.93 | 393.53 | 7871.12 | 473.94 |
| 923866 | Library | 141628.82 | 40321.68 | 4660.54 | 488.97 | 7717.51 | 2133.50 |
| 924645 | Library | 141648.77 | 21424.79 | 0.00 | 0.00 | 7376.18 | 2298.00 |
| 924649 | Library | 151580.59 | 45575.37 | 1090.82 | 299.97 | 7306.08 | 2078.08 |
| 923862 | Library | 209204.14 | 31225.39 | 3861.74 | 473.93 | 7280.19 | 822.12 |
| 924699 | Library | 148892.96 | 23157.90 | 0.00 | 0.00 | 7110.37 | 1081.80 |
| 924417 | Library | 151083.45 | 29342.93 | 1241.90 | 209.31 | 7004.79 | 495.30 |
| 924158 | Library | 209345.43 | 10454.44 | 4257.06 | 726.87 | 6934.64 | 631.05 |
| 923840 | Library | 174683.40 | 19391.32 | 3572.33 | 509.52 | 6876.76 | 886.61 |
| 923758 | Library | 145418.95 | 57553.10 | 5071.22 | 843.87 | 6840.99 | 2572.47 |
| 924719 | Library | 146068.50 | 19636.04 | 618.35 | 874.48 | 6826.34 | 788.19 |
| 924006 | Library | 189416.51 | 165132.62 | 5217.50 | 4527.21 | 6820.83 | 5910.11 |
| 924412 | Library | 195323.10 | 169219.33 | 4249.33 | 3691.43 | 6730.76 | 5829.49 |
| 924030 | Library | 178736.88 | 64076.70 | 2038.22 | 2882.48 | 6479.01 | 1812.50 |
| 924094 | Library | 188068.39 | 2330.96 | 3779.20 | 528.18 | 6459.52 | 699.12 |
| 924484 | Library | 159280.39 | 138551.56 | 5717.71 | 5011.47 | 6421.18 | 5605.67 |
| 924715 | Library | 125804.53 | 6613.14 | 0.00 | 0.00 | 6367.93 | 392.61 |
| 924576 | Library | 152154.76 | 11318.28 | 0.00 | 0.00 | 5891.04 | 531.59 |
| 924332 | Library | 144177.87 | 6454.12 | 3895.97 | 42.66 | 5833.44 | 207.35 |
| 924298 | Library | 160138.96 | 29655.26 | 3699.42 | 302.97 | 5660.25 | 562.07 |
| 924083 | Library | 217583.17 | 1486.77 | 1000.00 | 39.41 | 5623.26 | 186.33 |
| 924847 | Library | 217580.17 | 31871.05 | 6218.30 | 1078.95 | 5583.54 | 7896.32 |
| 924322 | Library | 288217.00 | 37468.18 | 8605.61 | 922.19 | 5499.29 | 7777.17 |
| 923811 | Library | 26641.51 | 9095.10 | 112727.53 | 27884.39 | 5443.54 | 1815.02 |
| 924707 | Library | 145605.92 | 43995.29 | 571.07 | 807.62 | 5431.56 | 623.45 |
| 924928 | Library | 32819.54 | 1624.43 | 107007.35 | 3934.89 | 5422.86 | 222.16 |
| 923848 | Library | 163629.18 | 51188.16 | 1571.77 | 2222.82 | 5391.12 | 2510.02 |
| 924266 | Library | 142442.09 | 24415.61 | 3307.49 | 0.28 | 5101.92 | 922.02 |
| 924661 | Library | 171514.73 | 19945.41 | 4302.42 | 758.79 | 4937.92 | 6983.27 |
| 924744 | Library | 19386.71 | 9073.00 | 56584.25 | 29855.81 | 4830.55 | 2949.83 |
| 924748 | Library | 15134.45 | 1189.07 | 66017.48 | 2294.99 | 4746.15 | 321.31 |
| 924828 | Library | 22525.99 | 4823.05 | 105742.32 | 20757.40 | 4612.11 | 1013.49 |
| 924492 | Library | 128800.63 | 19944.04 | 2307.33 | 238.46 | 4610.81 | 871.22 |
| 923695 | Library | 10378.27 | 97.26 | 42573.49 | 1473.10 | 4325.13 | 154.21 |
| 924932 | Library | 4006.46 | 2622.40 | 59884.77 | 29657.99 | 4201.31 | 2661.28 |
| 924446 | Library | 120067.03 | 109487.93 | 3128.32 | 2897.46 | 4200.80 | 3832.20 |
| 924348 | Library | 178267.94 | 37594.62 | 0.00 | 0.00 | 4186.63 | 5920.79 |
| 923786 | Library | 16627.11 | 201.62 | 40451.84 | 511.21 | 4061.00 | 241.84 |
| 924942 | Library | 2255.78 | 244.41 | 8942.86 | 572.33 | 4048.80 | 356.14 |
| 923960 | Library | 16410.92 | 3245.89 | 69267.79 | 1457.61 | 4043.56 | 744.71 |
| 924284 | Library | 21246.14 | 3298.50 | 91910.11 | 9211.85 | 3992.13 | 15.65 |
| 924502 | Library | 116614.01 | 24040.28 | 2177.13 | 488.39 | 3920.97 | 1479.93 |
| 923727 | Library | 8726.51 | 379.04 | 42966.45 | 1559.05 | 3892.30 | 186.73 |
| 924413 | Library | 24584.70 | 13004.62 | 103952.64 | 69718.49 | 3815.33 | 5395.69 |
| 924406 | Library | 105857.33 | 16443.31 | 989.48 | 1399.33 | 3716.50 | 706.10 |
| 924843 | Library | 22873.18 | 7051.32 | 86422.92 | 20608.95 | 3682.70 | 1384.92 |
| 924965 | Library | 19838.97 | 4568.87 | 57232.27 | 13286.44 | 3611.82 | 737.12 |
| 924864 | Library | 20898.87 | 6914.90 | 72009.96 | 21429.59 | 3502.55 | 1232.46 |
| 924940 | Library | 20619.74 | 3393.10 | 70128.26 | 10145.52 | 3386.07 | 679.19 |
| 923653 | Library | 7811.67 | 519.03 | 29363.67 | 2673.75 | 3347.14 | 642.22 |
| 923780 | Library | 200806.27 | 109422.29 | 4642.63 | 2067.01 | 3181.38 | 4499.15 |
| 923850 | Library | 2975.69 | 323.40 | 58344.98 | 6494.13 | 3139.06 | 152.49 |
| 923957 | Library | 16208.39 | 3558.88 | 70960.44 | 13965.88 | 2934.22 | 889.74 |
| 924836 | Library | 18336.57 | 15485.48 | 59766.12 | 43781.15 | 2896.55 | 4096.34 |
| 924642 | Library | 18179.53 | 6818.09 | 68334.78 | 15322.57 | 2880.71 | 640.93 |
| 924888 | Library | 3896.65 | 1022.21 | 60525.56 | 11422.11 | 2842.22 | 877.10 |
| 923910 | Library | 2623.20 | 247.23 | 56789.62 | 5066.97 | 2839.55 | 204.19 |
| 924618 | Library | 3244.66 | 3113.77 | 51680.12 | 48164.54 | 2739.45 | 2736.62 |
| 923896 | Library | 10828.69 | 15314.08 | 43198.07 | 61091.29 | 2730.93 | 3862.11 |
| 924760 | Library | 13383.48 | 401.49 | 61885.19 | 1211.31 | 2719.94 | 82.26 |
| 923899 | Library | 8061.46 | 2194.93 | 33292.87 | 4984.45 | 2659.66 | 887.64 |
| 924436 | Library | 149036.78 | 33430.96 | 2210.59 | 399.01 | 2634.74 | 3726.08 |
| 924890 | Library | 3701.39 | 219.49 | 63516.40 | 1356.07 | 2621.40 | 248.14 |
| 924240 | Library | 113456.82 | 163.07 | 0.00 | 0.00 | 2564.29 | 3626.45 |
| 924108 | Library | 4037.10 | 757.81 | 67461.46 | 12656.19 | 2540.32 | 571.21 |
| 924586 | Library | 250149.27 | 94025.70 | 1797.95 | 1557.31 | 2535.55 | 4391.71 |
| 924114 | Library | 4377.79 | 2115.93 | 45678.55 | 2980.23 | 2487.42 | 263.04 |
| 924051 | Library | 20151.52 | 995.90 | 93992.93 | 13561.97 | 2453.51 | 30.44 |
| 924765 | Library | 15014.48 | 776.47 | 42255.34 | 4104.24 | 2356.13 | 157.16 |
| 924801 | Library | 19848.37 | 13607.25 | 77605.22 | 57082.26 | 2263.18 | 3200.62 |
| 924264 | Library | 11453.67 | 3248.04 | 54038.49 | 10175.79 | 2248.23 | 453.47 |
| 923611 | Library | 11367.88 | 4061.75 | 43366.66 | 8924.48 | 2227.86 | 435.26 |
| 924640 | Library | 10808.98 | 8330.93 | 45323.48 | 30496.52 | 2135.33 | 1701.18 |
| 924747 | Library | 11661.08 | 3422.91 | 42644.35 | 14036.11 | 2091.24 | 651.81 |
| 924912 | Library | 9721.93 | 443.70 | 26344.36 | 993.87 | 2086.42 | 63.42 |
| 924767 | Library | 3011.51 | 4258.92 | 67936.30 | 60817.88 | 2049.86 | 2898.94 |
| 924524 | Library | 175383.63 | 104116.79 | 857.37 | 1212.50 | 1994.41 | 2820.52 |
| 924381 | Library | 12368.29 | 622.14 | 53395.15 | 14066.35 | 1973.97 | 264.44 |
| 924946 | Library | 7181.67 | 1372.03 | 20102.62 | 155.80 | 1901.03 | 1.27 |
| 924945 | Library | 2522.85 | 973.75 | 7246.77 | 2791.38 | 1896.45 | 688.02 |
| 924764 | Library | 8591.86 | 723.69 | 32645.88 | 1165.59 | 1828.40 | 123.76 |
| 924378 | Library | 10735.00 | 4082.09 | 32360.69 | 11228.57 | 1751.48 | 726.49 |
| 865957 | Library | 9341.86 | 518.06 | 39441.13 | 2674.35 | 1593.90 | 149.06 |
| 924716 | Library | 10414.59 | 2059.53 | 33697.39 | 4894.31 | 1590.67 | 333.41 |
| 924528 | Library | 17317.75 | 2231.86 | 65458.30 | 1979.12 | 1527.75 | 2160.56 |
| 923805 | Library | 5336.20 | 229.09 | 36817.63 | 1404.64 | 1476.25 | 5.04 |
| 924856 | Library | 2713.25 | 1726.12 | 56942.31 | 33375.02 | 1471.68 | 2081.27 |
| 923765 | Library | 11473.14 | 2075.13 | 57927.65 | 2591.21 | 1438.78 | 225.56 |
| 924112 | Library | 3946.74 | 5581.53 | 55603.81 | 39343.20 | 1393.66 | 1970.93 |
| 924253 | Library | 11286.69 | 4365.21 | 53666.00 | 2662.62 | 1351.28 | 1910.99 |
| 924246 | Library | 11314.51 | 3084.16 | 50886.96 | 15471.34 | 1332.44 | 1884.36 |
| 923672 | Library | 8977.54 | 2516.32 | 30635.58 | 9078.73 | 1322.83 | 406.46 |
| 924957 | Library | 1922.64 | 480.41 | 30921.30 | 5078.30 | 1283.90 | 368.76 |
| 924497 | Library | 7298.39 | 713.32 | 29283.96 | 2894.55 | 1272.30 | 143.34 |
| 923818 | Library | 9488.77 | 4484.81 | 48477.07 | 12851.12 | 1258.97 | 434.25 |
| 923807 | Library | 7156.69 | 1274.12 | 36798.09 | 102.96 | 1210.20 | 1711.49 |
| 924909 | Library | 10405.39 | 3428.74 | 50774.61 | 13056.61 | 1161.57 | 1642.71 |
| 923942 | Library | 1060.50 | 1499.78 | 35882.34 | 10776.51 | 1137.06 | 1608.04 |
| 924745 | Library | 7515.13 | 3563.10 | 25309.39 | 9023.09 | 1132.19 | 542.60 |
| 924131 | Library | 7808.78 | 3370.21 | 36057.68 | 12398.62 | 1121.68 | 379.85 |
| 924110 | Library | 1533.46 | 2168.64 | 46517.40 | 7959.31 | 1077.67 | 1524.05 |
| 924227 | Library | 0.00 | 0.00 | 26963.69 | 4050.19 | 1072.40 | 1516.61 |
| 924978 | Library | 6924.10 | 185.40 | 22577.89 | 740.86 | 1034.73 | 4.06 |
| 923657 | Library | 7548.42 | 1494.26 | 47546.48 | 3212.52 | 1033.49 | 144.57 |
| 924139 | Library | 1018.72 | 7.23 | 28464.76 | 238.28 | 1007.48 | 3.59 |
| 924688 | Library | 6065.57 | 2554.09 | 21581.49 | 7491.21 | 1001.90 | 408.61 |
| 924400 | Library | 7143.98 | 57.27 | 32550.65 | 1472.94 | 954.76 | 9.16 |
| 923726 | Library | 4512.32 | 1032.85 | 48798.98 | 3199.41 | 904.28 | 1278.84 |
| 924898 | Library | 0.00 | 0.00 | 9112.00 | 4159.12 | 889.47 | 323.58 |
| 923697 | Library | 7204.75 | 9013.27 | 35973.07 | 46750.56 | 839.93 | 1187.83 |
| 924969 | Library | 8329.31 | 2359.99 | 28656.83 | 6120.74 | 817.76 | 1156.49 |
| 923723 | Library | 4815.55 | 477.13 | 34719.27 | 4516.27 | 769.16 | 37.46 |
| 924167 | Library | 3076.17 | 2476.89 | 35760.69 | 1015.93 | 675.00 | 954.59 |
| 924660 | Library | 2365.55 | 1705.05 | 41801.18 | 29413.86 | 645.33 | 1290.67 |
| 923675 | Library | 5159.63 | 3797.88 | 27367.31 | 16021.65 | 622.98 | 881.03 |
| 924766 | Library | 12267.14 | 2282.32 | 52810.16 | 9379.68 | 578.92 | 1002.72 |
| 924261 | Library | 1532.60 | 3065.20 | 27254.65 | 32409.81 | 575.35 | 1150.70 |
| 924962 | Library | 5159.23 | 2171.15 | 19730.72 | 7310.86 | 502.52 | 710.67 |
| 924743 | Library | 4808.91 | 1146.99 | 17528.12 | 4721.24 | 418.14 | 591.34 |
| 924900 | Library | 3059.80 | 86.65 | 8998.25 | 307.50 | 398.36 | 563.36 |
| 924384 | Library | 7547.12 | 5042.83 | 30747.75 | 21685.56 | 379.88 | 759.76 |
| 924622 | Library | 5319.00 | 2630.02 | 11419.11 | 3856.85 | 325.23 | 650.46 |
| 924604 | Library | 414.80 | 829.60 | 20410.83 | 10410.89 | 280.46 | 560.93 |
| 924433 | Library | 430995.73 | 36695.66 | 4284.47 | 122.90 | 0.00 | 0.00 |
| 924487 | Library | 401026.29 | 63023.41 | 3075.52 | 450.49 | 0.00 | 0.00 |
| 924548 | Library | 365124.38 | 117135.37 | 3488.79 | 915.39 | 0.00 | 0.00 |
| 924829 | Library | 324182.74 | 27933.28 | 2064.03 | 128.12 | 0.00 | 0.00 |
| 924835 | Library | 298372.62 | 27271.36 | 869.74 | 1229.99 | 0.00 | 0.00 |
| 924851 | Library | 294348.43 | 9926.58 | 4879.82 | 115.45 | 0.00 | 0.00 |
| 924555 | Library | 294060.71 | 20117.07 | 8708.25 | 643.35 | 0.00 | 0.00 |
| 923609 | Library | 282714.54 | 68795.57 | 0.00 | 0.00 | 0.00 | 0.00 |
| 924552 | Library | 279763.40 | 10839.70 | 2114.17 | 84.16 | 0.00 | 0.00 |
| 924683 | Library | 271117.01 | 48148.66 | 4529.98 | 908.85 | 0.00 | 0.00 |
| 924455 | Library | 254534.28 | 155976.55 | 1316.64 | 1862.02 | 0.00 | 0.00 |
| 924587 | Library | 234962.20 | 167.38 | 7276.34 | 379.81 | 0.00 | 0.00 |
| 924580 | Library | 233927.11 | 224028.48 | 2208.55 | 2185.30 | 0.00 | 0.00 |
| 924811 | Library | 230432.89 | 1724.05 | 1379.80 | 145.74 | 0.00 | 0.00 |
| 924855 | Library | 228608.15 | 15644.17 | 673.69 | 952.74 | 0.00 | 0.00 |
| 924218 | Library | 226224.56 | 84423.25 | 6461.18 | 2791.48 | 0.00 | 0.00 |
| 924214 | Library | 216983.50 | 37021.79 | 5090.58 | 734.80 | 0.00 | 0.00 |
| 924459 | Library | 216127.95 | 6790.04 | 2040.12 | 167.87 | 0.00 | 0.00 |
| 924520 | Library | 214973.13 | 293604.37 | 1873.08 | 2648.94 | 0.00 | 0.00 |
| 924346 | Library | 211486.66 | 1276.33 | 4404.74 | 156.97 | 0.00 | 0.00 |
| 923784 | Library | 198602.92 | 172006.67 | 3189.08 | 2764.01 | 0.00 | 0.00 |
| 924681 | Library | 195073.71 | | 1436.61 | | 0.00 | |
| 924585 | Library | 185168.35 | 107032.48 | 3082.36 | 1896.19 | 0.00 | 0.00 |
| 924522 | Library | 175962.87 | 48850.64 | 763.64 | 1079.95 | 0.00 | 0.00 |
| 924262 | Library | 175386.42 | 14122.77 | 3625.15 | 132.34 | 0.00 | 0.00 |
| 924693 | Library | 173644.31 | 67898.01 | 2315.44 | 763.60 | 0.00 | 0.00 |
| 924686 | Library | 170785.53 | 15851.96 | 1454.22 | 206.30 | 0.00 | 0.00 |
| 924583 | Library | 155402.97 | 99318.93 | 2190.27 | 1128.01 | 0.00 | 0.00 |
| 924595 | Library | 154197.84 | 108543.73 | 3402.88 | 2227.33 | 0.00 | 0.00 |
| 924867 | Library | 12187.37 | 955.28 | 44660.13 | 5231.35 | 0.00 | 0.00 |
| 924407 | Library | 10661.73 | | 52587.25 | | 0.00 | |
| 924352 | Library | 8154.51 | 5780.53 | 38228.82 | 21075.56 | 0.00 | 0.00 |
| 923928 | Library | 7823.27 | 362.71 | 42638.93 | 3643.80 | 0.00 | 0.00 |
| 923647 | Library | 7733.74 | 2779.99 | 38088.66 | 10881.98 | 0.00 | 0.00 |
| 924808 | Library | 7719.31 | 1119.16 | 38166.94 | 875.50 | 0.00 | 0.00 |
| 924500 | Library | 7625.27 | 1680.07 | 43355.58 | 8035.93 | 0.00 | 0.00 |
| 923917 | Library | 7503.97 | 960.47 | 34185.42 | 5596.98 | 0.00 | 0.00 |
| 923694 | Library | 7485.64 | 711.92 | 41799.49 | 8127.03 | 0.00 | 0.00 |
| 923924 | Library | 6904.45 | 3022.57 | 38431.85 | 13720.88 | 0.00 | 0.00 |
| 923610 | Library | 6804.19 | 2593.47 | 40862.43 | 16332.21 | 0.00 | 0.00 |
| 923851 | Library | 6684.07 | 362.65 | 34201.15 | 584.86 | 0.00 | 0.00 |
| 923679 | Library | 6627.09 | 2934.60 | 36281.96 | 13776.09 | 0.00 | 0.00 |
| 923806 | Library | 6621.06 | 54.51 | 29425.87 | 3951.47 | 0.00 | 0.00 |
| 923603 | Library | 6480.64 | 1671.41 | 33241.91 | 2174.66 | 0.00 | 0.00 |
| 923870 | Library | 6271.39 | 220.19 | 35402.17 | 1658.19 | 0.00 | 0.00 |
| 923836 | Library | 5830.25 | 1115.66 | 24933.50 | 5014.90 | 0.00 | 0.00 |
| 923935 | Library | 5598.95 | 1929.11 | 29095.63 | 8813.97 | 0.00 | 0.00 |
| 923946 | Library | 5388.59 | 1337.33 | 28487.59 | 2199.89 | 0.00 | 0.00 |
| 923617 | Library | 4246.87 | 73.64 | 27179.49 | 988.73 | 0.00 | 0.00 |
| 924979 | Library | 3227.29 | 214.40 | 12558.33 | 626.81 | 0.00 | 0.00 |
| 924920 | Library | 3207.79 | 151.61 | 10970.07 | 897.15 | 0.00 | 0.00 |
| 924960 | Library | 3010.63 | 582.61 | 11081.72 | 2710.55 | 0.00 | 0.00 |
| 924684 | Library | 2701.20 | 400.87 | 8836.56 | 843.45 | 0.00 | 0.00 |
| 924908 | Library | 2498.30 | 131.37 | 11608.96 | 1721.00 | 0.00 | 0.00 |
| 924652 | Library | 2090.85 | 102.19 | 7211.25 | 107.23 | 0.00 | 0.00 |
| 924778 | Library | 1595.92 | 24.36 | 35350.64 | 928.52 | 0.00 | 0.00 |
| 924800 | Library | 1259.61 | 1781.36 | 37017.45 | 18808.93 | 0.00 | 0.00 |
| 923978 | Library | 1010.74 | 1429.41 | 34018.82 | 3615.96 | 0.00 | 0.00 |
| 924812 | Library | 485.71 | 686.90 | 7670.80 | 1029.75 | 0.00 | 0.00 |
| 923953 | Library | 0.00 | 0.00 | 26226.92 | 2662.12 | 0.00 | 0.00 |
| 923955 | Library | 0.00 | 0.00 | 28579.54 | 2078.92 | 0.00 | 0.00 |
| 923974 | Library | 0.00 | 0.00 | 36616.89 | 432.55 | 0.00 | 0.00 |
| 924746 | Library | 0.00 | 0.00 | 14713.52 | 2056.79 | 0.00 | 0.00 |
| 924910 | Library | 0.00 | 0.00 | 8994.34 | 1423.24 | 0.00 | 0.00 |
| 924922 | Library | 0.00 | 0.00 | 22921.59 | 4575.63 | 0.00 | 0.00 |
| 924929 | Library | 0.00 | 0.00 | 13879.23 | 3834.36 | 0.00 | 0.00 |
| 924961 | Library | 0.00 | 0.00 | 8092.36 | 2467.09 | 0.00 | 0.00 |
| 924972 | Library | 0.00 | 0.00 | 10150.46 | 685.25 | 0.00 | 0.00 |
| 924973 | Library | 0.00 | 0.00 | 6970.13 | 1056.22 | 0.00 | 0.00 |
| 924977 | Library | 0.00 | 0.00 | 8814.39 | 1239.69 | 0.00 | 0.00 |

TABLE 16B
Percent Terminal C6 Product Titers of terminal
synthase candidate enzymes in S. cerevisiae

| | Fold difference | Fold difference | | | |
|---|---|---|---|---|---|
| | over Average | over Average | | | |
| Strain | THCA from | CBDA from | % | % | % |
| ID | strain t865977 | strain t865859 | THCA | CBDA | CBCA |
| 865859 | | 1.02 | 16.49 | 81.82 | 1.69 |
| 865977 | 0.95 | | 97.18 | 0.42 | 2.40 |
| 876606 | 0.39 | | 98.85 | 0.11 | 1.04 |
| 876607 | | 0.25 | 0.51 | 99.49 | 0.00 |
| 923976 | 2.34 | | 58.31 | 0.96 | 40.73 |
| 923759 | 3.69 | | 73.75 | 0.65 | 25.60 |
| 923624 | | | 59.34 | 0.00 | 40.66 |
| 923980 | | | 74.24 | 0.00 | 25.76 |
| 923922 | | | 71.53 | 0.00 | 28.47 |
| 923890 | | | 69.99 | 0.00 | 30.01 |
| 923616 | | | 71.93 | 0.61 | 27.46 |
| 923954 | | | 72.31 | 0.00 | 27.69 |
| 923894 | | | 58.91 | 0.00 | 41.09 |
| 923972 | 3.13 | | 86.41 | 2.75 | 10.84 |
| 923680 | | | 67.61 | 1.01 | 31.38 |
| 923918 | | | 72.71 | 0.00 | 27.29 |
| 924465 | 3.96 | | 92.51 | 2.50 | 4.98 |
| 924725 | 5.16 | | 94.61 | 0.00 | 5.39 |
| 924927 | 2.97 | | 95.24 | 0.00 | 4.76 |
| 923908 | 4.58 | | 92.52 | 3.04 | 4.44 |
| 923613 | | | 73.37 | 0.00 | 26.63 |
| 924717 | 5.00 | | 94.70 | 0.00 | 5.30 |
| 924309 | | | 83.06 | 6.90 | 10.04 |
| 923795 | | | 72.95 | 0.00 | 27.05 |
| 923880 | 4.32 | | 93.98 | 2.00 | 4.02 |
| 924364 | 4.23 | | 94.76 | 0.00 | 5.24 |
| 924612 | | | 91.42 | 1.26 | 7.32 |
| 924164 | | | 84.05 | 6.95 | 9.00 |
| 924803 | | | 89.33 | 0.74 | 9.94 |
| 924559 | 3.56 | | 93.44 | 1.73 | 4.82 |
| 924468 | 5.55 | | 92.02 | 3.95 | 4.03 |
| 923884 | 3.95 | | 93.10 | 3.08 | 3.82 |
| 924276 | 4.57 | | 94.40 | 1.92 | 3.68 |
| 924665 | 3.40 | | 94.99 | 0.00 | 5.01 |
| 924691 | | | 84.18 | 6.54 | 9.29 |
| 924639 | 3.28 | | 95.07 | 0.00 | 4.93 |
| 924512 | 3.52 | | 94.94 | 0.00 | 5.06 |
| 924723 | 3.51 | | 94.98 | 0.00 | 5.02 |
| 923916 | 3.24 | | 93.48 | 2.42 | 4.10 |
| 924428 | 4.24 | | 93.85 | 2.71 | 3.44 |
| 924472 | 4.95 | | 92.22 | 3.96 | 3.82 |
| 924609 | 3.22 | | 93.02 | 2.69 | 4.29 |
| 924657 | 2.31 | | 91.73 | 2.49 | 5.78 |
| 924226 | 3.93 | | 94.33 | 2.00 | 3.67 |
| 924306 | 3.42 | | 93.62 | 2.13 | 4.25 |
| 924695 | 3.22 | | 94.04 | 0.78 | 5.17 |
| 924162 | 3.28 | | 92.92 | 2.89 | 4.19 |
| 924387 | 4.99 | | 95.89 | 0.78 | 3.32 |
| 924667 | 3.14 | | 94.99 | 0.39 | 4.62 |
| 924425 | 2.96 | | 95.33 | 0.88 | 3.79 |
| 924373 | 2.95 | | 94.38 | 0.79 | 4.83 |
| 924212 | 3.91 | | 92.45 | 3.21 | 4.34 |
| 924635 | 2.91 | | 94.76 | 0.38 | 4.86 |
| 923912 | 3.42 | | 93.99 | 2.52 | 3.49 |
| 924304 | 2.62 | | 91.96 | 3.02 | 5.02 |
| 924028 | 2.60 | | 93.52 | 2.47 | 4.01 |
| 924426 | 3.10 | | 92.65 | 3.34 | 4.00 |
| 924160 | 3.36 | | 94.30 | 1.97 | 3.74 |
| 924424 | 3.78 | | 95.03 | 1.64 | 3.33 |
| 924431 | 2.31 | | 94.82 | 0.89 | 4.29 |
| 924518 | 2.59 | | 95.29 | 0.92 | 3.79 |
| 923771 | 3.73 | | 96.97 | 0.46 | 2.57 |
| 923744 | 3.93 | | 95.15 | 1.90 | 2.95 |
| 924673 | 2.22 | | 94.42 | 0.00 | 5.58 |
| 924398 | 2.70 | | 93.09 | 2.67 | 4.24 |
| 923948 | 2.52 | | 93.06 | 3.01 | 3.94 |
| 924062 | 2.50 | | 94.73 | 1.74 | 3.53 |
| 923854 | 3.60 | | 92.31 | 3.22 | 4.47 |
| 923866 | 3.14 | | 91.96 | 3.03 | 5.01 |
| 924645 | 2.18 | | 95.05 | 0.00 | 4.95 |
| 924649 | 2.33 | | 94.75 | 0.68 | 4.57 |
| 923862 | 4.63 | | 94.94 | 1.75 | 3.30 |
| 924699 | 2.29 | | 95.44 | 0.00 | 4.56 |
| 924417 | 2.58 | | 94.82 | 0.78 | 4.40 |
| 924158 | 3.03 | | 94.93 | 1.93 | 3.14 |
| 923840 | 2.31 | | 94.36 | 1.93 | 3.71 |
| 923758 | 3.22 | | 92.43 | 3.22 | 4.35 |
| 924719 | 2.50 | | 95.15 | 0.40 | 4.45 |
| 924006 | 2.24 | | 94.02 | 2.59 | 3.39 |
| 924412 | 3.74 | | 94.68 | 2.06 | 3.26 |
| 924030 | 2.68 | | 95.45 | 1.09 | 3.46 |
| 924094 | 2.22 | | 94.84 | 1.91 | 3.26 |
| 924484 | 2.30 | | 92.92 | 3.34 | 3.75 |
| 924715 | 2.15 | | 95.18 | 0.00 | 4.82 |
| 924576 | 2.60 | | 96.27 | 0.00 | 3.73 |
| 924332 | 2.16 | | 93.68 | 2.53 | 3.79 |
| 924298 | 2.40 | | 94.48 | 2.18 | 3.34 |
| 924083 | 2.38 | | 97.05 | 0.45 | 2.51 |
| 924847 | 2.93 | | 94.85 | 2.71 | 2.43 |
| 924322 | 3.46 | | 95.33 | 2.85 | 1.82 |
| 923811 | | 2.52 | 18.40 | 77.84 | 3.76 |
| 924707 | 2.24 | | 96.04 | 0.38 | 3.58 |
| 924928 | | 3.58 | 22.60 | 73.67 | 3.73 |
| 923848 | 2.16 | | 95.92 | 0.92 | 3.16 |
| 924266 | 2.13 | | 94.43 | 2.19 | 3.38 |
| 924661 | 2.36 | | 94.89 | 2.38 | 2.73 |
| 924744 | | 4.15 | 23.99 | 70.03 | 5.98 |
| 924748 | | 4.85 | 17.62 | 76.86 | 5.53 |
| 924828 | | 1.57 | 16.95 | 79.58 | 3.47 |
| 924492 | 2.47 | | 94.90 | 1.70 | 3.40 |
| 923695 | | 0.76 | 18.12 | 74.33 | 7.55 |
| 924932 | | 1.27 | 5.88 | 87.95 | 6.17 |
| 924446 | 2.30 | | 94.25 | 2.46 | 3.30 |
| 924348 | 2.14 | | 97.71 | 0.00 | 2.29 |
| 923786 | | 0.85 | 27.20 | 66.16 | 6.64 |
| 924942 | | 0.66 | 14.79 | 58.65 | 26.55 |
| 923960 | | 1.20 | 18.29 | 77.20 | 4.51 |
| 924284 | | 1.42 | 18.14 | 78.46 | 3.41 |
| 924502 | 2.23 | | 95.03 | 1.77 | 3.20 |
| 923727 | | 0.76 | 15.70 | 77.30 | 7.00 |
| 924413 | | 1.62 | 18.58 | 78.54 | 2.88 |
| 924406 | 2.03 | | 95.74 | 0.89 | 3.36 |
| 924843 | | 1.84 | 20.25 | 76.49 | 3.26 |
| 924965 | | 1.22 | 24.59 | 70.93 | 4.48 |
| 924864 | | 1.53 | 21.68 | 74.69 | 3.63 |
| 924940 | | 5.15 | 21.90 | 74.50 | 3.60 |
| 923653 | | 0.60 | 19.28 | 72.46 | 8.26 |
| 923780 | 2.95 | | 96.25 | 2.23 | 1.52 |
| 923850 | | 1.07 | 4.62 | 90.51 | 4.87 |
| 923957 | | 1.45 | 17.99 | 78.75 | 3.26 |
| 924836 | | 0.89 | 22.64 | 73.79 | 3.58 |
| 924642 | | 1.36 | 20.34 | 76.44 | 3.22 |
| 924888 | | 1.29 | 5.79 | 89.98 | 4.23 |
| 923910 | | 1.04 | 4.21 | 91.22 | 4.56 |
| 924618 | | 0.80 | 5.63 | 89.62 | 4.75 |
| 923896 | | 0.75 | 19.08 | 76.11 | 4.81 |
| 924760 | | 0.92 | 17.16 | 79.35 | 3.49 |
| 923899 | | 0.68 | 18.32 | 75.64 | 6.04 |
| 924436 | 2.05 | | 96.85 | 1.44 | 1.71 |
| 924890 | | 1.35 | 5.30 | 90.95 | 3.75 |
| 924240 | 2.12 | | 97.79 | 0.00 | 2.21 |
| 924108 | | 1.16 | 5.45 | 91.12 | 3.43 |
| 924586 | 2.95 | | 98.30 | 0.71 | 1.00 |
| 924114 | | 0.79 | 8.33 | 86.93 | 4.73 |
| 924051 | | 1.85 | 17.28 | 80.61 | 2.10 |
| 924765 | | 3.10 | 25.18 | 70.87 | 3.95 |
| 924801 | | 1.02 | 19.90 | 77.83 | 2.27 |
| 924264 | | 0.84 | 16.9 | 79.77 | 3.32 |
| 923611 | | 0.77 | 19.96 | 76.13 | 3.91 |
| 924640 | | 0.90 | 18.55 | 77.78 | 3.66 |
| 924747 | | 3.13 | 20.68 | 75.62 | 3.71 |
| 924912 | | 0.56 | 25.48 | 69.05 | 5.47 |
| 924767 | | 0.93 | 4.13 | 93.07 | 2.81 |
| 924524 | 2.10 | | 98.40 | 0.48 | 1.12 |
| 924381 | | 1.07 | 18.26 | 78.83 | 2.91 |
| 924946 | | 1.48 | 24.61 | 68.88 | 6.51 |
| 924945 | | 0.53 | 21.63 | 62.12 | 16.26 |
| 924764 | | 2.40 | 19.95 | 75.80 | 4.25 |
| 924378 | | 0.64 | 23.94 | 72.16 | 3.91 |
| 865957 | | 0.71 | 18.54 | 78.29 | 3.16 |
| 924716 | | 2.47 | 22.79 | 73.73 | 3.48 |
| 924528 | | 1.02 | 20.54 | 77.65 | 1.81 |
| 923805 | | 0.72 | 12.23 | 84.39 | 3.38 |
| 924856 | | 0.78 | 4.44 | 93.15 | 2.41 |
| 923765 | | 1.18 | 16.20 | 81.77 | 2.03 |
| 924112 | | 0.97 | 6.48 | 91.24 | 2.29 |
| 924253 | | 1.03 | 17.02 | 80.94 | 2.04 |
| 924246 | | 0.85 | 17.81 | 80.09 | 2.10 |
| 923672 | | 0.64 | 21.93 | 74.84 | 3.23 |
| 924957 | | 2.27 | 5.63 | 90.60 | 3.76 |
| 924497 | | 0.58 | 19.28 | 77.36 | 3.36 |
| 923818 | | 0.98 | 16.02 | 81.85 | 2.13 |
| 923807 | | 0.79 | 15.85 | 81.47 | 2.68 |
| 924909 | | 0.56 | 16.69 | 81.45 | 1.86 |
| 923942 | | 0.66 | 2.78 | 94.23 | 2.99 |
| 924745 | | 1.86 | 22.13 | 74.53 | 3.33 |
| 924131 | | 0.66 | 17.36 | 80.15 | 2.49 |
| 924110 | | 0.81 | 3.12 | 94.69 | 2.19 |
| 924227 | | 0.52 | 0.00 | 96.17 | 3.83 |
| 924978 | | 1.66 | 22.67 | 73.94 | 3.39 |
| 923657 | | 0.92 | 13.45 | 84.71 | 1.84 |
| 924139 | | 0.56 | 3.34 | 93.35 | 3.30 |
| 924688 | | 1.58 | 21.17 | 75.33 | 3.50 |
| 924400 | | 0.65 | 17.57 | 80.08 | 2.35 |
| 923726 | | 1.18 | 8.32 | 90.01 | 1.67 |
| 924898 | | 0.67 | 0.00 | 91.11 | 8.89 |
| 923697 | | 0.73 | 16.3″ | 81.72 | 1.91 |
| 924969 | | 0.61 | 22.03 | 75.80 | 2.16 |
| 923723 | | 0.68 | 11.95 | 86.14 | 1.91 |
| 924167 | | 0.64 | 7.79 | 90.51 | 1.71 |
| 924660 | | 1.47 | 5.28 | 93.28 | 1.44 |
| 923675 | | 0.56 | 15.56 | 82.56 | 1.88 |
| 924766 | | 1.58 | 18.68 | 80.43 | 0.88 |
| 924261 | | 0.50 | 5.22 | 92.82 | 1.96 |
| 924962 | | 1.45 | 20.32 | 77.70 | 1.98 |
| 924743 | | 1.29 | 21.13 | 77.03 | 1.84 |
| 924900 | | 0.66 | 24.56 | 72.24 | 3.20 |
| 924384 | | 0.51 | 19.51 | 79.50 | 0.98 |
| 924622 | | 0.56 | 31.17 | 66.92 | 1.91 |
| 924604 | | 0.77 | 1.97 | 96.71 | 1.33 |
| 924433 | 4.54 | | 99.02 | 0.98 | 0.00 |
| 924487 | 4.80 | | 99.24 | 0.76 | 0.00 |
| 924548 | 3.84 | | 99.05 | 0.95 | 0.00 |
| 924829 | 3.59 | | 99.37 | 0.63 | 0.00 |
| 924835 | 3.30 | | 99.71 | 0.29 | 0.00 |
| 92485 | 3.96 | | 98.37 | 1.63 | 0.00 |
| 924555 | 4.13 | | 97.12 | 2.88 | 0.00 |
| 923609 | 4.24 | | 100.00 | 0.00 | 0.00 |
| 924552 | 2.95 | | 99.25 | 0.75 | 0.00 |
| 924683 | 3.73 | | 98.36 | 1.64 | 0.00 |
| 924455 | 3.04 | | 99.49 | 0.51 | 0.00 |
| 924587 | 3.30 | | 97.00 | 3.00 | 0.00 |
| 924580 | 2.46 | | 99.06 | 0.94 | 0.00 |
| 924811 | 2.55 | | 99.40 | 0.60 | 0.00 |
| 924855 | 2.53 | | 99.71 | 0.29 | 0.00 |
| 924218 | 2.72 | | 97.22 | 2.78 | 0.00 |
| 924214 | 4.06 | | 97.71 | 2.29 | 0.00 |
| 924459 | 2.58 | | 99.06 | 0.94 | 0.00 |
| 924520 | 2.57 | | 99.14 | 0.86 | 0.00 |
| 924346 | 2.54 | | 97.96 | 2.04 | 0.00 |
| 923784 | 2.92 | | 98.42 | 1.58 | 0.00 |
| 924681 | 2.33 | | 99.27 | 0.73 | 0.00 |
| 924585 | 2.60 | | 98.36 | 1.64 | 0.00 |
| 924522 | 2.10 | | 99.57 | 0.43 | 0.00 |
| 924262 | 2.11 | | 97.97 | 2.03 | 0.00 |
| 924693 | 2.39 | | 98.68 | 1.32 | 0.00 |
| 924686 | 2.12 | | 99.16 | 0.84 | 0.00 |
| 924583 | 2.18 | | 98.61 | 1.39 | 0.00 |
| 924595 | 2.12 | | 97.84 | 2.16 | 0.00 |
| 924867 | | 0.61 | 21.44 | 78.56 | 0.00 |
| 924407 | | 0.82 | 16.86 | 83.14 | 0.00 |
| 924352 | | 0.59 | 17.58 | 82.42 | 0.00 |
| 923928 | | 0.74 | 15.50 | 84.50 | 0.00 |
| 923647 | | 0.78 | 16.88 | 83.12 | 0.00 |
| 924808 | | 0.57 | 16.82 | 83.18 | 0.00 |
| 924500 | | 0.71 | 14.96 | 85.04 | 0.00 |
| 923917 | | 0.76 | 18.00 | 82.00 | 0.00 |
| 923694 | | 1.01 | 15.19 | 84.81 | 0.00 |
| 923924 | | 0.66 | 15.23 | 84.77 | 0.00 |
| 923610 | | 0.98 | 14.27 | 85.73 | 0.00 |
| 923851 | | 0.76 | 16.35 | 83.65 | 0.00 |
| 923679 | | 0.74 | 15.44 | 84.56 | 0.00 |
| 923806 | | 0.54 | 18.37 | 81.63 | 0.00 |
| 923603 | | 0.59 | 16.31 | 83.69 | 0.00 |
| 923870 | | 0.65 | 15.05 | 84.95 | 0.00 |
| 923836 | | 0.60 | 18.95 | 81.05 | 0.00 |
| 923935 | | 0.65 | 16.14 | 83.86 | 0.00 |
| 923946 | | 0.52 | 15.91 | 84.09 | 0.00 |
| 923617 | | 0.55 | 13.51 | 86.49 | 0.00 |
| 924979 | | 0.92 | 20.44 | 79.56 | 0.00 |
| 924920 | | 0.81 | 22.63 | 77.37 | 0.00 |
| 924960 | | 0.81 | 21.36 | 78.64 | 0.00 |
| 924684 | | 0.65 | 23.41 | 76.59 | 0.00 |
| 924908 | | 0.85 | 17.71 | 82.29 | 0.00 |
| 924652 | | 0.53 | 22.48 | 77.52 | 0.00 |
| 924778 | | 0.52 | 4.32 | 95.68 | 0.00 |
| 924800 | | 0.55 | 3.29 | 96.71 | 0.00 |
| 923978 | | 0.60 | 2.89 | 97.11 | 0.00 |
| 924812 | | 0.56 | 5.95 | 94.05 | 0.00 |
| 923953 | | 0.53 | 0.00 | 100.00 | 0.00 |
| 923955 | | 0.58 | 0.00 | 100.00 | 0.00 |
| 923974 | | 0.65 | 0.00 | 100.00 | 0.00 |
| 924746 | | 1.08 | 0.00 | 100.00 | 0.00 |
| 924910 | | 0.66 | 0.00 | 100.00 | 0.00 |
| 924922 | | 1.68 | 0.00 | 100.00 | 0.00 |
| 924929 | | 1.02 | 0.00 | 100.00 | 0.00 |
| 924961 | | 0.59 | 0.00 | 100.00 | 0.00 |
| 924972 | | 0.75 | 0.00 | 100.00 | 0.00 |
| 924973 | | 0.51 | 0.00 | 100.00 | 0.00 |
| 924977 | | 0.65 | 0.00 | 100.00 | 0.00 |

TABLE 17
Amino Acid Substitutions in Terminal Synthases Described in Example 3

| | Nucleic | Amino | | |
|---|---|---|---|---|
| Strain | acid SEQ | acid SEQ | Reference | Amino Acid Substitutions Relative to Reference |
| ID | ID NO: | ID NO: | Sequence | Sequence |
| 616314 | 45 | 136 | — | — |
| 701909 | 46 | 137 | SEQ ID | E424D E425K A430T L443V T446I A447C 1459L |
| | | | NO: 14 | V462I S464N V4651 T469M Q475K L489I K491I |
| | | | (UniProt: | T492N N493D H494P A495K S496N N516D K524L |
| | | | Q8GTB6) | P542R H544R |
| 701916 | 47 | 138 | SEQ ID | R31Q K40E H41Y V46P L51F V52I 163V I74T |
| | | | NO: 14 | N90V T96S V103I A116S P542L |
| | | | (UniProt: | |
| | | | Q8GTB6) | |
| 701917 | 48 | 139 | SEQ ID | E40Q N44T P46V A47T P49A F51L 152V L59F |
| | | | NO: 20 | V631 V85I S88L A95G S96T K165N F169L G173A |
| | | | (UniProt: | V181A H208Q K247R K254M G268E F273V |
| | | | I1V0C5) | K282M D284E D286E V288L M290F K296R H302Q |
| | | | | V309I G311S E344Q F345L T351I V357L F360Y |
| | | | | A363T A375G K377R T379A T395S A396V M397F |
| | | | | K399Q V409I V411A V415A E424D M440L T446I |
| | | | | A447C I459L V462I S464N V465I T469M Q475K |
| | | | | L489I K491I T492N N493D E495K S496N N516D |
| | | | | K524L A525V P542R H544R |
| 701919 | 49 | 140 | SEQ ID | V288L |
| | | | NO: 14 | |
| | | | (UniProt: | |
| | | | Q8GTB6) | |
| 701934 | 50 | 141 | SEQ ID | |
| | | | NO: 14 | |
| | | | (UniProt: | |
| | | | Q8GTB6) | |
| 701940 | 51 | 142 | SEQ ID | Q31R E40K Y41H P46V F51L I52V V63L T74I |
| | | | NO: 20 | V90N S96T K165N F169L G173A V181A H208Q |
| | | | (UniProt: | K247R K254M G268E F273V K282M D284E D286E |
| | | | I1V0C5) | V288L M290F K296R H302Q V3091 G311S E344Q |
| | | | | F345L T351I V357L F360Y A363T A375G K377R |
| | | | | T379A |
| 701964 | 52 | 143 | SEQ ID | H69Q |
| | | | NO: 13 | |
| | | | (UniProt: | |
| | | | A6P6V9) | |
| 701992 | 53 | 144 | SEQ ID | N89D |
| | | | NO: 14 | |
| | | | (UniProt: | |
| | | | Q8GTB6) | |
| 701998 | 54 | 145 | SEQ ID | R31Q K40E H41Y V46P L51F V52I I63V I74T |
| | | | NO: 14 | N90V T96S V103I A116S P542L |
| | | | (UniProt: | |
| | | | Q8GTB6) | |
| 702004 | 55 | 146 | SEQ ID | E40Q N44T P46V A47T P49A F5IL I52V L59F |
| | | | NO: 20 | V63I V85I S88L A95G S96T K165N F169L G173A |
| | | | (UniProt: | V181A H208Q K247R K254M G268E F273V |
| | | | I1V0C5) | K282M D284E D286E V288L M290F K296R H302Q |
| | | | | V309I G311S E344Q F345L T351I V357L F360Y |
| | | | | A363T A375G K377R T379A |
| 702008 | 56 | 147 | SEQ ID | B40Q N44T P46V A47T P49A F51L I52V L59F |
| | | | NO: 20 | V631 V85I S88L A95G S96T K165N F169L G173A |
| | | | (UniProt: | V181A H208Q K247R K254M G268E F273V |
| | | | I1V0C5) | K282M D284E D286E V288L M290F K296R H302Q |
| | | | | V309I G311S E344Q F345L T351I V357L F360Y |
| | | | | A363T A375G K377R T379A |
| 702022 | 57 | 148 | SEQ ID | Q3IR E40K Y41H P46V F51L I52V V63L T74I |
| | | | NO: 20 | V90N S96T K165N F169L G173A V181A H208Q |
| | | | (UniProt: | K247R K254M G268E F273V K282M D284E D286E |
| | | | I1V0C5) | V288L M290F K296R H302Q V309I G311S E344Q |
| | | | | F345L T351I V357L F360Y A363T A375G K377R |
| | | | | T379A |
| 702056 | 58 | 149 | SEQ ID | H69R |
| | | | NO: 13 | |
| | | | (UniProt: | |
| | | | A6P6V9) | |
| 702080 | 59 | 150 | SEQ ID | I63L L443I T446I V462I S464N L479M H494F |
| | | | NO: 14 | A495E N528D P542L H543R |
| | | | (UniProt: | |
| | | | Q8GTB6) | |
| 702105 | 60 | 151 | SEQ ID | A410T |
| | | | NO: 13 | |
| | | | (UniProt: | |
| | | | A6P6V9) | |
| 702109 | 61 | 152 | SEQ ID | A410V |
| | | | NO: 13 | |
| | | | (UniProt: | |
| | | | A6P6V9) | |
| 702115 | 62 | 153 | SEQ ID | G378S |
| | | | NO: 13 | |
| | | | (UniProt: | |
| | | | A6P6V9) | |
| 702118 | 63 | 154 | SEQ ID | I63L L443I T446I V462I S464N L479M H494F |
| | | | NO: 14 | A495E N528D P542L H543R |
| | | | (UniProt: | |
| | | | Q8GTB6) | |
| 702123 | 64 | 155 | SEQ ID | R31Q K40Q H41Y N44T A47T P49A L59F I74T |
| | | | NO: 14 | V85I S88L N90V A95G P542L H543R |
| | | | (UniProt: | |
| | | | Q8GTB6) | |
| 702136 | 65 | 156 | SEQ ID | H213N |
| | | | NO: 13 | |
| | | | (UniProt: | |
| | | | A6P6V9) | |
| 702147 | 66 | 157 | SEQ ID | E424D E425K A430T L443V T446I A447C I459L |
| | | | NO: 14 | V462I S464N V465I T469M Q475K L489I K491I |
| | | | (UniProt: | T492N N493D H494P A495K S496N N516D K524L |
| | | | Q8GTB6) | P542R H544R |
| 702150 | 67 | 158 | SEQ ID | |
| | | | NO: 14 | |
| | | | (UniProt: | |
| | | | Q8GTB6) | |
| 702155 | 68 | 159 | SEQ ID | E40Q N44T P46V A47T P49A F51L I52V L59F |
| | | | NO: 20 | V631 V85I S88L A95G S96T K165N F169L G173A |
| | | | (UniProt: | V181A H208Q K247R K254M G268E F273V |
| | | | I1V0C5) | K282M D284E D286E V288L M290F K296R H302Q |
| | | | | V309I G311S E344Q F345L T351I V357L F360Y |
| | | | | A363T A375G K377R T379A T395S A396V M397F |
| | | | | K399Q V409I V411A V415A E424D M440L T446I |
| | | | | A447C I459L V462I S464N V465I T469M Q475K |
| | | | | L489I K491I T492N N493D E495K S496N N516D |
| | | | | K524L A525V PS42R H544R |
| 702187 | 69 | 160 | SEQ ID | G378T |
| | | | NO: 13 | |
| | | | (UniProt: | |
| | | | A6P6V9) | |
| 702201 | 70 | 161 | SEQ ID | E424D E425K A430T L443V T446I A447C I459L |
| | | | NO: 14 | V462I S464N V465I T469M Q475K L489I K491I |
| | | | (UniProt: | T492N N493D H494P A495K S496N N516D K524L |
| | | | Q8GTB6) | P542R H544R |
| 702215 | 71 | 162 | SEQ ID | Q31R E40K Y41H P46V F51L I52V V63L T74I |
| | | | NO: 20 | V90N S96T K165N F169L G173A V181A H208Q |
| | | | (UniProt: | K247R K254M G268E F273V K282M D284E D286E |
| | | | I1V0CS) | V288L M290F K296R H302Q V309I G311S E344Q |
| | | | | F345L T351I V357L F360Y A363T A375G K377R |
| | | | | T379A T395S A396V M397F K399Q V409I V411A |
| | | | | V415A E424D M440L T446I A447C I459L V462I |
| | | | | S464N V465I T469M Q475K L489I K491I T492N |
| | | | | N493D E495K S496N N516D K524L A525V P542R |
| | | | | H544R |
| 702257 | 72 | 163 | SEQ ID | T339E |
| | | | NO: 13 | |
| | | | (UniProt: | |
| | | | A6P6V9) | |
| 702258 | 73 | 164 | SEQ ID | R31Q K40Q H41Y I74T N90V V129I V288L K296R |
| | | | NO: 14 | F345L F360Y A411V E424D H494P A495E |
| | | | (UniProt: | |
| | | | Q8GTB6) | |
| 702261 | 74 | 165 | SEQ ID | G319D |
| | | | NO: 13 | |
| | | | (UniProt: | |
| | | | A6P6V9) | |
| 702276 | 75 | 166 | SEQ ID | H89D |
| | | | NO: 13 | |
| | | | (UniProt: | |
| | | | A6P6V9) | |
| 702278 | 76 | 167 | SEQ ID | Q31R E40K Y41H P46V F51L I52V V63L T74I |
| | | | NO: 20 | V90N S96T K165N F169L G173A V181A H208Q |
| | | | (UniProt: | K247R K254M G268E F273V K282M D284E D286E |
| | | | I1V0C5) | V288L M290F K296R H302Q V309I G311S E344Q |
| | | | | F345L T351I V357L F360Y A363T A375G K377R |
| | | | | T379A T395S A396V M397F K399Q V409I V411A |
| | | | | V415A E424D M440L T446I A447C I459L V462I |
| | | | | S464N V465I T469M Q475K L489I K491I T492N |
| | | | | N493D E495K S496N N516D K524L A525V P542R |
| | | | | H544R |
| 702280 | 77 | 168 | SEQ ID | H89E |
| | | | NO: 13 | |
| | | | (UniProt: | |
| | | | A6P6V9) | |
| 702288 | 78 | 169 | SEQ ID | M289F |
| | | | NO: 13 | |
| | | | (UniProt: | |
| | | | A6P6V9) | |
| 702297 | 79 | 170 | SEQ ID | G378R |
| | | | NO: 13 | |
| | | | (UniProt: | |
| | | | A6P6V9) | |
| 702304 | 80 | 171 | SEQ ID | H89Q |
| | | | NO: 13 | |
| | | | (UniProt: | |
| | | | A6P6V9) | |
| 702308 | 81 | 172 | SEQ ID | H89R |
| | | | NO: 13 | |
| | | | (UniProt: | |
| | | | A6P6V9) | |
| 702315 | 82 | 173 | SEQ ID | Y353M |
| | | | NO: 13 | |
| | | | (UniProt: | |
| | | | A6P6V9) | |
| 702329 | 83 | 174 | SEQ ID | I382S |
| | | | NO: 13 | |
| | | | (UniProt: | |
| | | | A6P6V9) | |
| 702338 | 84 | 175 | SEQ ID | A414I |
| | | | NO: 13 | |
| | | | (UniProt: | |
| | | | A6P6V9) | |
| 702342 | 85 | 176 | SEQ ID | A414L |
| | | | NO: 13 | |
| | | | (UniProt: | |
| | | | A6P6V9) | |
| 702346 | 86 | 177 | SEQ ID | A414M |
| | | | NO: 13 | |
| | | | (UniProt: | |
| | | | A6P6V9) | |
| 702350 | 87 | 178 | SEQ ID | R31Q K40Q H41Y V46A A47T P49A H56N Q58P |
| | | | NO: 14 | I63V I74T N90V A95G V129I H136R G173A |
| | | | (UniProt: | V181A N237S A242V K247R I257M G268E F273V |
| | | | Q8GTB6) | V288L K296R H302Q V309I G311S H318L E344Q |
| | | | | F345L T351I F360Y N361D A363T K377Q K378N |
| | | | | T379A S382K A396V A411V E424D T446I I459L |
| | | | | V462I S464N T469M T492N H494P A495K P542R |
| | | | | H544R |
| 702370 | 88 | 179 | SEQ ID | Y416F |
| | | | NO: 13 | |
| | | | (UniProt: | |
| | | | A6P6V9) | |
| 702376 | 89 | 180 | SEQ ID | S116A |
| | | | NO: 13 | |
| | | | (UniProt: | |
| | | | A6P6V9) | |
| 702396 | 90 | 181 | SEQ ID | M289W |
| | | | NO: 13 | |
| | | | (UniProt: | |
| | | | A6P6V9) | |
| 702412 | 91 | 182 | SEQ ID | S116G |
| | | | NO: 13 | |
| | | | (UniProt: | |
| | | | A6P6V9) | |
| 702462 | 92 | 183 | SEQ ID | Y416I |
| | | | NO: 13 | |
| | | | (UniProt: | |
| | | | A6P6V9) | |
| 702470 | 93 | 184 | SEQ ID | Y416M |
| | | | NO: 13 | |
| | | | (UniProt: | |
| | | | A6P6V9) | |
| 702485 | 94 | 185 | SEQ ID | N168T |
| | | | NO: 13 | |
| | | | (UniProt: | |
| | | | A6P6V9) | |
| 702507 | 95 | 186 | SEQ ID | H143E |
| | | | NO: 13 | |
| | | | (UniProt: | |
| | | | A6P6V9) | |
| 702513 | 96 | 187 | SEQ ID | Q106E |
| | | | NO: 13 | |
| | | | (UniProt: | |
| | | | A6P6V9) | |
| 702517 | 97 | 188 | SEQ ID | L344M |
| | | | NO: 13 | |
| | | | (UniProt: | |
| | | | A6P6V9) | |
| 702531 | 98 | 189 | SEQ ID | Q376L |
| | | | NO: 13 | |
| | | | (UniProt: | |
| | | | A6P6V9) | |
| 702563 | 99 | 190 | SEQ ID | Q376Y |
| | | | NO: 13 | |
| | | | (UniProt: | |
| | | | A6P6V9) | |
| 702571 | 100 | 191 | SEQ ID | G378K |
| | | | NO: 13 | |
| | | | (UniProt: | |
| | | | A6P6V9) | |
| 702581 | 101 | 192 | SEQ ID | A414T |
| | | | NO: 13 | |
| | | | (UniProt: | |
| | | | A6P6V9) | |
| 702585 | 102 | 193 | SEQ ID | A414V |
| | | | NO: 13 | |
| | | | (UniProt: | |
| | | | A6P6V9) | |
| 702591 | 103 | 194 | SEQ ID | V397E |
| | | | NO: 13 | |
| | | | (UniProt: | |
| | | | A6P6V9) | |
| 702595 | 104 | 195 | SEQ ID | S100A |
| | | | NO: 13 | |
| | | | (UniProt: | |
| | | | A6P6V9) | |
| 702601 | 105 | 196 | SEQ ID | E40Q P46A A49S P51A F53L I54V H58N Q60P |
| | | | NO: 20 | A97G S98T V131I H138R F171L G175A V183A |
| | | | (UniProt: | N239S A244V K249R I259M G270E F275V V290L |
| | | | I1V0C5) | K298R H304Q V311I G313S H320L E346Q F347L |
| | | | | T353I F362Y N363D A365T K379Q K380N T381A |
| | | | | S384K A398V E426D T448I I461L V464I S466N |
| | | | | T471M T494N E497K A527V P544R H546R |
| 702603 | 106 | 197 | SEQ ID | M263L |
| | | | NO: 13 | |
| | | | (UniProt: | |
| | | | A6P6V9) | |
| 702660 | 107 | 198 | SEQ ID | R31Q K40Q H41Y N44T A47T P49A L59F I74T |
| | | | NO: 14 | V85I S88L N90V A95G P542L H543R |
| | | | (UniProt: | |
| | | | Q8GTB6) | |
| 702688 | 108 | 199 | SEQ ID | I63L L443I T446I V462I S464N L479M H494F |
| | | | NO: 14 | A495E N528D P542L H543R |
| | | | (UniProt: | |
| | | | Q8GTB6) | |
| 702891 | 109 | 200 | SEQ ID | P542R H544R |
| | | | NO: 14 | |
| | | | (UniProt: | |
| | | | Q8GTB6) | |
| 702948 | 110 | 201 | SEQ ID | L287T |
| | | | NO: 13 | |
| | | | (UniProt: | |
| | | | A6P6V9) | |
| 703131 | 111 | 202 | SEQ ID | I445V |
| | | | NO: 13 | |
| | | | (UniProt: | |
| | | | A6P6V9) | |
| 703178 | 112 | 203 | SEQ ID | A250D |
| | | | NO: 14 | |
| | | | (UniProt: | |
| | | | Q8GTB6) | |
| 703300 | 113 | 204 | SEQ ID | E4418 |
| | | | NO: 13 | |
| | | | (UniProt: | |
| | | | A6P6V9) | |
| 703306 | 114 | 205 | SEQ ID | E44IT |
| | | | NO: 13 | |
| | | | (UniProt: | |
| | | | A6P6V9) | |
| 703341 | 115 | 206 | SEQ ID | Q31R E40K Y41H P46V F51L I52V V63L T74I |
| | | | NO: 20 | V90N S96T K165N F169L G173A V181A H208Q |
| | | | (UniProt: | K247R K254M G268E F273V K282M D284E D286E |
| | | | I1V0C5) | V288L M290F K296R H302Q V309I G311S E344Q |
| | | | | F345L T351I V357L F360Y A363T A375G K377R |
| | | | | T379A |
| 703452 | 116 | 207 | SEQ ID | V397K |
| | | | NO: 13 | |
| | | | (UniProt: | |
| | | | A6P6V9) | |
| 703455 | 117 | 208 | SEQ ID | N454A |
| | | | NO: 13 | |
| | | | (UniProt: | |
| | | | A6P6V9) | |
| 703459 | 118 | 209 | SEQ ID | V103H |
| | | | NO: 13 | |
| | | | (UniProt: | |
| | | | A6P6V9) | |
| 703473 | 119 | 210 | SEQ ID | N527D |
| | | | NO: 13 | |
| | | | (UniProt: | |
| | | | A6P6V9) | |
| 703482 | 120 | 211 | SEQ ID | G319N |
| | | | NO: 13 | |
| | | | (UniProt: | |
| | | | A6P6V9) | |
| 703520 | 121 | 212 | SEQ ID | Q124N |
| | | | NO: 13 | |
| | | | (UniProt: | |
| | | | A6P6V9) | |
| 703524 | 122 | 213 | SEQ ID | A250R |
| | | | NO: 13 | |
| | | | (UniProt: | |
| | | | A6P6V9) | |
| 703528 | 123 | 214 | SEQ ID | V216L |
| | | | NO: 13 | |
| | | | (UniProt: | |
| | | | A6P6V9) | |
| 703584 | 124 | 215 | SEQ ID | E167K |
| | | | NO: 13 | |
| | | | (UniProt: | |
| | | | A6P6V9) | |
| 703607 | 125 | 216 | SEQ ID | K512N |
| | | | NO: 13 | |
| | | | (UniProt: | |
| | | | A6P6V9) | |
| 703611 | 126 | 217 | SEQ ID | K450S |
| | | | NO: 13 | |
| | | | (UniProt: | |
| | | | A6P6V9) | |
| 703634 | 127 | 218 | SEQ ID | D407E |
| | | | NO: 13 | |
| | | | (UniProt: | |
| | | | A6P6V9) | |
| 703638 | 128 | 219 | SEQ ID | D283P |
| | | | NO: 13 | |
| | | | (UniProt: | |
| | | | A6P6V9) | |
| 703685 | 129 | 220 | SEQ ID | G95A |
| | | | NO: 13 | |
| | | | (UniProt: | |
| | | | A6P6V9) | |
| 703699 | 130 | 221 | SEQ ID | N166S |
| | | | NO: 13 | |
| | | | (UniProt: | |
| | | | A6P6V9) | |
| 703703 | 131 | 222 | SEQ ID | I490T |
| | | | NO: 13 | |
| | | | (UniProt: | |
| | | | A6P6V9) | |
| 703707 | 132 | 223 | SEQ ID | S322E |
| | | | NO: 13 | |
| | | | (UniProt: | |
| | | | A6P6V9) | |
| 703721 | 133 | 224 | SEQ ID | S394E |
| | | | NO: 13 | |
| | | | (UniProt: | |
| | | | A6P6V9) | |
| 703738 | 134 | 225 | SEQ ID | K50N |
| | | | NO: 13 | |
| | | | (UniProt: | |
| | | | A6P6V9) | |
| 616315 | 22 | 21 | — | — |
| 701870 | 254 | 284 | — | — |
| 701939 | 255 | 285 | SEQ ID | V288M |
| | | | NO: 14 | |
| | | | (UniProt: | |
| | | | Q8GTB6) | |
| 701954 | 256 | 286 | SEQ ID | A250D |
| | | | NO: 14 | |
| | | | (UniProt: | |
| | | | Q8GTB6) | |
| 701963 | 257 | 287 | SEQ ID | P542R H544R |
| | | | NO: 14 | |
| | | | (UniProt: | |
| | | | Q8GTB6) | |
| 701977 | 258 | 288 | SEQ ID | R31Q K40Q H41Y N44T A47T P49A L59F I74T |
| | | | NO: 14 | V85I S88L N90V A95G P542L H543R |
| | | | (UniProt: | |
| | | | Q8GTB6) | |
| 701990 | 259 | 289 | SEQ ID | I63L P542L |
| | | | NO: 14 | |
| | | | (UniProt: | |
| | | | Q8GTB6) | |
| 701996 | 260 | 290 | SEQ ID | N89E |
| | | | NO: 14 | |
| | | | (UniProt: | |
| | | | Q8GTB6) | |
| 702000 | 261 | 291 | SEQ ID | N89H |
| | | | NO: 14 | |
| | | | (UniProt: | |
| | | | Q8GTB6) | |
| 702043 | 262 | 292 | SEQ ID | V288T |
| | | | NO: 14 | |
| | | | (UniProt: | |
| | | | Q8GTB6) | |
| 702050 | 263 | 293 | SEQ ID | R31Q K40Q H41Y N44T A47T P49A L59F I74T |
| | | | NO: 14 | V85I S88L N90V A95G P542L H543R |
| | | | (UniProt: | |
| | | | Q8GTB6) | |
| 702054 | 264 | 294 | SEQ ID | I63L P542L |
| | | | NO: 14 | |
| | | | (UniProt: | |
| | | | Q8GTB6) | |
| 702090 | 265 | 295 | SEQ ID | A250D |
| | | | NO: 14 | |
| | | | (UniProt: | |
| | | | Q8GTB6) | |
| 702154 | 266 | 296 | SEQ ID | A250D |
| | | | NO: 14 | |
| | | | (UniProt: | |
| | | | Q8GTB6) | |
| 702232 | 267 | 297 | SEQ ID | I63L P542L |
| | | | NO: 14 | |
| | | | (UniProt: | |
| | | | Q8GTB6) | |
| 702240 | 268 | 298 | SEQ ID | R31Q K40Q H41Y N44T A47T P49A L59F I74T |
| | | | NO: 14 | V85I S88L N90V A95G PS42L H543R |
| | | | (UniProt: | |
| | | | Q8GTB6) | |
| 702761 | 269 | 299 | SEQ ID | N61Q N301Q |
| | | | NO: 20 | |
| | | | (UniProt: | |
| | | | I1V0C5) | |
| 702767 | 270 | 300 | SEQ ID | N61Q N301Q N495Q |
| | | | NO: 20 | |
| | | | (UniProt: | |
| | | | IIVoC5) | |
| 702801 | 271 | 301 | SEQ ID | N61Q N301Q N325Q |
| | | | NO: 20 | |
| | | | (UniProt: | |
| | | | I1V0C5) | |
| 702894 | 272 | 302 | SEQ ID | H494D |
| | | | NO: 14 | |
| | | | (UniProt: | |
| | | | Q8GTB6) | |
| 702927 | 273 | 303 | SEQ ID | N61Q N164Q |
| | | | NO: 20 | |
| | | | (UniProt: | |
| | | | I1V0C5) | |
| 702942 | 274 | 304 | SEQ ID | H494E |
| | | | NO: 14 | |
| | | | (UniProt: | |
| | | | Q8GTB6) | |
| 702993 | 275 | 305 | SEQ ID | I63L P542L |
| | | | NO: 14 | |
| | | | (UniProt: | |
| | | | Q8GTB6) | |
| 703174 | 276 | 306 | SEQ ID | |
| | | | NO: 14 | |
| | | | (UniProt: | |
| | | | Q8GTB6) | |
| 703239 | 277 | 307 | SEQ ID | T469M |
| | | | NO: 14 | |
| | | | (UniProt: | |
| | | | Q8GTB6) | |
| 703256 | 278 | 308 | SEQ ID | I63L P542L |
| | | | NO: 14 | |
| | | | (UniProt: | |
| | | | Q8GTB6) | |
| 703289 | 279 | 309 | SEQ ID | N329Q |
| | | | NO: 14 | |
| | | | (UniProt: | |
| | | | Q8GTB6) | |
| 703637 | 280 | 310 | SEQ ID | N499Q |
| | | | NO: 14 | |
| | | | (UniProt: | |
| | | | Q8GTB6) | |
| 703690 | 281 | 311 | SEQ ID | M61S |
| | | | NO: 14 | |
| | | | (UniProt: | |
| | | | Q8GTB6) | |
| 703722 | 282 | 312 | SEQ ID | N90E |
| | | | NO: 14 | |
| | | | (UniProt: | |
| | | | Q8GTB6) | |
| 703725 | 283 | 313 | SEQ ID | H136R |
| | | | NO: 14 | |
| | | | (UniProt: | |
| | | | Q8GTB6) | |

TABLE 18
Amino Acid Substitutions in Terminal Synthases Described in Example 4

| | Nucleic | Amino | Reference | |
|---|---|---|---|---|
| Strain | acid SEQ | acid SEQ | Sequence | Amino Acid Substitutions Relative to Reference |
| ID | ID NO: | ID NO: | ID | Sequence |
| t825123 | 322 | 464 | SEQ ID NO: | A414V |
| | | | 13 (UniProt: | |
| | | | A6P6V9) | |
| t825215 | 323 | 465 | SEQ ID NO: | A414V |
| | | | 13 (UniProt: | |
| | | | A6P6V9) | |
| t825585 | 324 | 466 | SEQ ID NO: | A414V |
| | | | 13 (UniProt: | |
| | | | A6P6V9) | |
| t826070 | 325 | 467 | SEQ ID NO: | S116A |
| | | | 13 (UniProt: | |
| | | | A6P6V9) | |
| t826072 | 326 | 468 | SEQ ID NO: | S116A |
| | | | 13 (UniProt: | |
| | | | A6P6V9) | |
| t826076 | 327 | 469 | SEQ ID NO: | S116A |
| | | | 13 (UniProt: | |
| | | | A6P6V9) | |
| t826096 | 328 | 470 | SEQ ID NO: | S116A |
| | | | 13 (UniProt: | |
| | | | A6P6V9) | |
| t825125 | 329 | 471 | SEQ ID NO: | K50N |
| | | | 13 (UniProt: | |
| | | | A6P6V9) | |
| t825t89 | 330 | 472 | SEQ ID NO: | K50N |
| | | | 13 (UniProt: | |
| | | | A6P6V9) | |
| t825217 | 331 | 473 | SEQ ID NO: | K50N |
| | | | 13 (UniProt: | |
| | | | A6P6V9) | |
| t825219 | 332 | 474 | SEQ ID NO: | R31Q K40Q H41Y N44T A47T P49A L59F I74T V85I |
| | | | 14 (UniProt: | S88L N90V A95G P542L H543R |
| | | | Q8GTB6) | |
| t825377 | 333 | 475 | SEQ ID NO: | R31Q K40Q H41Y N44T A47T P49A L59F I74T V85I |
| | | | 14 (UniProt: | S88L N90V A95G P542L H543R |
| | | | Q8GTB6) | |
| t826030 | 334 | 476 | SEQ ID NO: | R31Q K40Q H41Y N44T A47T P49A L59F I74T V85I |
| | | | 14 (UniProt: | S88L N90V A95G P542L H543R |
| | | | Q8GTB6) | |
| t826036 | 335 | 477 | SEQ ID NO: | R31Q K40Q H41Y N44T A47T P49A L59F I74T V85I |
| | | | 14 (UniProt: | S88L N90V A95G P542L H543R |
| | | | Q8GTB6) | |
| t824622 | 336 | 478 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t825119 | 337 | 479 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t825129 | 338 | 480 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t825151 | 339 | 481 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t825213 | 340 | 482 | SEQ ID NO: | R3IQ K40Q H41Y I74T N90V V129I V288L K296R |
| | | | 14 (UniProt: | F345L F360Y A411V E424D H494P A495E |
| | | | Q8GTB6) | |
| t825221 | 341 | 483 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t825379 | 342 | 484 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t826054 | 343 | 485 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t826100 | 344 | 486 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t826132 | 345 | 487 | SEQ ID NO: | R31Q K40Q H41Y I74T N90V V129I V288L K296R |
| | | | 14 (UniProt: | F345L F360Y A411V E424D H494P A495E |
| | | | Q8GTB6) | |
| t824932 | 346 | 488 | SEQ ID NO: | R31Q K40Q H41Y H56N M61S I74T N90V V129I |
| | | | 14 (UniProt: | S255V V288L M290F K296R T340E F345L T351I |
| | | | Q8GTB6) | F360Y A411V E424D Q475K T492N H494P A495E |
| t825910 | 347 | 489 | SEQ ID NO: | R31Q K40Q H41Y V46P M61S I74T N90V V129I |
| | | | 14 (UniProt: | S255V V288L M290F K296R T340E F345L F360Y |
| | | | Q8GTB6) | A411V E424D T492N H494P A495E |
| t825025 | 348 | 490 | SEQ ID NO: | I74T N90V A250D S255V H494B |
| | | | 14 (UniProt: | |
| | | | Q8GTB6) | |
| t825621 | 349 | 491 | SEQ ID NO: | A250D S255V H494E |
| | | | 14 (UniProt: | |
| | | | Q8GTB6) | |
| t824996 | 350 | 492 | SEQ ID NO: | R31Q K40Q H41Y V46P H56N M618 I74T N90V |
| | | | 14 (UniProt: | V129I S255V V288L M290F K296R F345L F360Y |
| | | | Q8GTB6) | A411V E424D T492N H494P A495E |
| t824498 | 351 | 493 | SEQ ID NO: | R31Q K40Q H41Y V46P H56N M61S I74T N90V |
| | | | 14 (UniProt: | V129I H143E S255V V288L M290F K296R T340E |
| | | | Q8GTB6) | F345L Y354F F360Y A411V E424D Q475K T492N |
| | | | | H494P A495E |
| t825269 | 352 | 494 | SEQ ID NO: | R31Q K40Q H41Y V46P V52I H56N Q58S M61S I74T |
| | | | 14 (UniProt: | N90V V129I S255V V288L M290F K296R T340E |
| | | | Q8GTB6) | |
| | | | | F345L F360Y A411V E424D Q475K T492N H494P |
| | | | | A495E |
| t825259 | 353 | 495 | SEQ ID NO: | R31Q K40Q H41Y H56N Q58S M61S I74T N90V |
| | | | 14 (UniProt: | V129I S255V V288L M290F K296R T340E F345L |
| | | | Q8GTB6) | F360Y A411V E424D Q475K T492N H494P A495E |
| t825978 | 354 | 496 | SEQ ID NO: | R31Q K40Q H41Y M61S I74T N90V V129I S255V |
| | | | 14 (UniProt: | V288L M290F K296R T340E F345L F360Y A411V |
| | | | Q8GTB6) | E424D Q475K T492N H494P A495E |
| t825043 | 355 | 497 | SEQ ID NO: | R31Q K40Q H41Y Q58S M61S I74T N90V V129I |
| | | | 14 (UniProt: | H143E S255V V288L M290F K296R T340E F345L |
| | | | Q8GTB6) | F360Y A411V E424D Q475K T492N H494P A495E |
| t825528 | 356 | 498 | SEQ ID NO: | R31Q K40Q H41Y V46P H56N Q58S M61S I74T N90V |
| | | | 14 (UniProt: | V129I V158L S255V V288L M290F K296R T340E |
| | | | Q8GTB6) | F345L F360Y A411V E424D Q475K T492N H494P |
| | | | | A495E |
| t824930 | 357 | 499 | SEQ ID NO: | R31Q M61S I74T N90V A250P S255V E424D Q475K |
| | | | 14 (UniProt: | T492N H494E |
| | | | Q8GTB6) | |
| t825077 | 358 | 500 | SEQ ID NO: | R31Q K40Q H41Y I74T N90V V129I H143E S255V |
| | | | 14 (UniProt: | V288L M290F K296R F345L F360Y A411V E424D |
| | | | Q8GTB6) | T492N H494P A495E |
| t825023 | 359 | 501 | SEQ ID NO: | H56N I74T N90V A250D S255V T492N H494E |
| | | | 14 (UniProt: | |
| | | | Q8GTB6) | |
| t825103 | 360 | 502 | SEQ ID NO: | R31Q V46P I74T N90V A250D S255V E424D Q475K |
| | | | 14 (UniProt: | T492N H494E A495E |
| | | | Q8GTB6) | |
| t824618 | 361 | 503 | SEQ ID NO: | R31Q K40Q H41Y V46P H56N Q58S M61S I74T N90V |
| | | | 14 (UniProt: | V129I S255V V288L M290F K296R T340E F345L |
| | | | Q8GTB6) | F360Y A411V E424D T446I Q475K T492N H494P |
| | | | | A495E |
| t825071 | 362 | 504 | SEQ ID NO: | V46P I74T N90V A250D S255V |
| | | | 14 (UniProt: | |
| | | | Q8GTB6) | |
| t825059 | 363 | 505 | SEQ ID NO: | H56N M61S I74T N90V A250P S255V T492N H494E |
| | | | 14 (UniProt: | |
| | | | Q8GTB6) | |
| t825126 | 364 | 506 | SEQ ID NO: | K50N H69Q G95A H213N Q343E L344M |
| | | | 13 (UniProt: | |
| | | | A6P6V9) | |
| t825766 | 365 | 507 | SEQ ID NO: | K50N H69Q G95A H213N T339E L344M |
| | | | 13 (UniProt: | |
| | | | A6P6V9) | |
| t825987 | 366 | 508 | SEQ ID NO: | H69Q G95A S116A T339E |
| | | | 13 (UniProt: | |
| | | | A6P6V9) | |
| t826274 | 367 | 509 | SEQ ID NO: | H69Q G95A S116A T339E Q343E |
| | | | 13 (UniProt: | |
| | | | A6P6V9) | |
| t825341 | 368 | 510 | SEQ ID NO: | T47A L49P N56H N57D P58Q H69Q H89N G95A S253 |
| | | | 13 (UniProt: | insertion |
| | | | A6P6V9) | |
| t826093 | 369 | 511 | SEQ ID NO: | S100A S116A H213N |
| | | | 13 (UniProt: | |
| | | | A6P6V9) | |
| t825841 | 370 | 512 | SEQ ID NO: | G95A H213N T339E Q343E A414V D492N |
| | | | 13 (UniProt: | |
| | | | A6P6V9) | |
| t824918 | 371 | 513 | SEQ ID NO: | H69Q G95A S116A H213N T339E Q343E |
| | | | 13 (UniProt: | |
| | | | A6P6V9) | |
| t825034 | 372 | 514 | SEQ ID NO: | K50N H69Q G95A Q343E |
| | | | 13 (UniProt: | |
| | | | A6P6V9) | |
| t825593 | 373 | 515 | SEQ ID NO: | G95A T339E L344M A414V |
| | | | 13 (UniProt: | |
| | | | A6P6V9) | |
| t825277 | 374 | 516 | SEQ ID NO: | R31Q T47A L49P N56H N57D P58Q H69Q H89N |
| | | | 13 (UniProt: | G95A S253 insertion |
| | | | A6P6V9) | |
| t825833 | 375 | 517 | SEQ ID NO: | G95A T339E Q343E L344M A414V |
| | | | 13 (UniProt: | |
| | | | A6P6V9) | |
| t825936 | 376 | 518 | SEQ ID NO: | G95A S116A T339E Q343E L344M |
| | | | 13 (UniProt: | |
| | | | A6P6V9) | |
| t825933 | 377 | 519 | SEQ ID NO: | K50N H69Q G95A H213N Q343E L344M A414V |
| | | | 13 (UniProt: | |
| | | | A6P6V9) | |
| t824603 | 378 | 520 | SEQ ID NO: | K50N G95A S116A H213N L344M N527D |
| | | | 13 (UniProt: | |
| | | | A6P6V9) | |
| t824539 | 379 | 521 | SEQ ID NO: | K50N G95A H213N T339E Q343E A414V D492N |
| | | | 13 (UniProt: | |
| | | | A6P6V9) | |
| t824659 | 380 | 522 | SEQ ID NO: | G95A H213N T339E G378T A410V A414V I445V |
| | | | 13 (UniProt: | |
| | | | A6P6V9) | |
| t824773 | 381 | 523 | SEQ ID NO: | G95A S116A T339E |
| | | | 13 (UniProt: | |
| | | | A6P6V9) | |
| t825907 | 382 | 524 | SEQ ID NO: | G95A H213N Q343E L344M A414V |
| | | | 13 (UniProt: | |
| | | | A6P6V9) | |
| t825930 | 383 | 525 | SEQ ID NO: | G95A S116A H213N Q343E L344M |
| | | | 13 (UniProt: | |
| | | | A6P6V9) | |
| t826097 | 384 | 526 | SEQ ID NO: | K50N G95A A414V |
| | | | 13 (UniProt: | |
| | | | A6P6V9) | |
| t825889 | 385 | 527 | SEQ ID NO: | G95A H213N T339E L344M A414V |
| | | | 13 (UniProt: | |
| | | | A6P6V9) | |
| t824654 | 386 | 528 | SEQ ID NO: | K50N H69Q G95A Q343E A414V |
| | | | 13 (UniProt: | |
| | | | A6P6V9) | |
| t824571 | 387 | 529 | SEQ ID NO: | K50N H69Q G95A H213N T339E Q343E A414V |
| | | | 13 (UniProt: | |
| | | | A6P6V9) | |
| t825248 | 388 | 530 | SEQ ID NO: | G95A S116A Q343E |
| | | | 13 (UniProt: | |
| | | | A6P6V9) | |
| t824807 | 389 | 531 | SEQ ID NO: | G95A S116A H213N T339E Q343E |
| | | | 13 (UniProt: | |
| | | | A6P6V9) | |
| t825877 | 390 | 532 | SEQ ID NO: | G95A H213N Q343B A414V |
| | | | 13 (UniProt: | |
| | | | A6P6V9) | |
| t826099 | 391 | 533 | SEQ ID NO: | G95A Q343E G378T A414V |
| | | | 13 (UniProt: | |
| | | | A6P6V9) | |
| t824746 | 392 | 534 | SEQ ID NO: | H69Q G95A H213N T339E Q343E L344M A414V |
| | | | 13 (UniProt: | |
| | | | A6P6V9) | |
| t824612 | 393 | 535 | SEQ ID NO: | K50N G95A H213N Q343E L344M A414V |
| | | | 13 (UniProt: | |
| | | | A6P6V9) | |
| t824626 | 394 | 536 | SEQ ID NO: | H69Q G95A H213N L344M A414V |
| | | | 13 (UniProt: | |
| | | | A6P6V9) | |
| t825154 | 395 | 537 | SEQ ID NO: | G95A S116A T339E Q343E |
| | | | 13 (UniProt: | |
| | | | A6P6V9) | |
| t824540 | 396 | 538 | SEQ ID NO: | H69Q G95A Q343E A414V N527D |
| | | | 13 (UniProt: | |
| | | | A6P6V9) | |
| t824786 | 397 | 539 | SEQ ID NO: | H69Q G95A H213N S322E Q343E L344M A414V |
| | | | 13 (UniProt: | |
| | | | A6P6V9) | |
| t824688 | 398 | 540 | SEQ ID NO: | G95A S116A H213N T339E Q343E L344M |
| | | | 13 (UniProt: | |
| | | | A6P6V9) | |
| t825012 | 399 | 541 | SEQ ID NO: | K50N H69Q H213N T339E Q343E A414V |
| | | | 13 (UniProt: | |
| | | | A6P6V9) | |
| t824646 | 400 | 542 | SEQ ID NO: | H69Q G95A H213N Q343E A414V N527D |
| | | | 13 (UniProt: | |
| | | | A6P6V9) | |
| t825862 | 401 | 543 | SEQ ID NO: | K50N G95A Q343E L344M A414V |
| | | | 13 (UniProt: | |
| | | | A6P6V9) | |
| t824653 | 402 | 544 | SEQ ID NO: | K50N G95A H213N T339E Q343E L344M A414V |
| | | | 13 (UniProt: | |
| | | | A6P6V9) | |
| t824625 | 403 | 545 | SEQ ID NO: | G95A T339E Q343E A414V |
| | | | 13 (UniProt: | |
| | | | A6P6V9) | |
| t824942 | 404 | 546 | SEQ ID NO: | K50N H69Q G95A T339B Q343B L344M A414V |
| | | | 13 (UniProt: | |
| | | | A6P6V9) | |
| t825773 | 405 | 547 | SEQ ID NO: | G95A A414V N527D |
| | | | 13 (UniProt: | |
| | | | A6P6V9) | |
| t825108 | 406 | 548 | SEQ ID NO: | K50N H213N Q343E L344M A414V |
| | | | 13 (UniProt: | |
| | | | A6P6V9) | |
| t825301 | 407 | 549 | SEQ ID NO: | I74T N90V A250P S255V E424D T492N H494E A495E |
| | | | 14 (UniProt: | |
| | | | Q8GTB6) | |
| t825935 | 408 | 550 | SEQ ID NO: | G95A H213N T339E Q343E L344M A414V |
| | | | 13 (UniProt: | |
| | | | A6P6V9) | |
| t825739 | 409 | 551 | SEQ ID NO: | G95A Q343E L344M A414V |
| | | | 13 (UniProt: | |
| | | | A6P6V9) | |
| t825724 | 410 | 552 | SEQ ID NO: | G95A H213N T339E Q343E A414V |
| | | | 13 (UniProt: | |
| | | | A6P6V9) | |
| t824845 | 411 | 553 | SEQ ID NO: | M61S I74T N90V A250P S255V E424D T492N H494E |
| | | | 14 (UniProt: | |
| | | | Q8GTB6) | |
| t825099 | 412 | 554 | SEQ ID NO: | R31Q K40Q H41Y I74T N90V V129I V288L K296R |
| | | | 14 (UniProt: | F345L F360Y A411V E424D H494P A495E Y419F |
| | | | Q8GTB6) | |
| t825297 | 413 | 555 | SEQ ID NO: | R31Q I74T N90V A250D S255V T492N H494E |
| | | | 14 (UniProt: | |
| | | | Q8GTB6) | |
| t824990 | 414 | 556 | SEQ ID NO: | I74T N90V A250P H494E |
| | | | 14 (UniProt: | |
| | | | Q8GTB6) | |
| t824771 | 415 | 557 | SEQ ID NO: | R31Q K40Q H41Y N44T A47T P49A L59F M61S I74T |
| | | | 14 (UniProt: | V85I S88L N90V A95G H143E S255V E424D T492N |
| | | | Q8GTB6) | H494E PS42L H543R |
| t825263 | 416 | 558 | SEQ ID NO: | R31Q K40Q H41Y V46P I74T N90V V129I V288L |
| | | | 14 (UniProt: | K296R T340E F345L F360Y A411V E424D H494P |
| | | | Q8GTB6) | A495E |
| t825286 | 417 | 559 | SEQ ID NO: | M61S I74T N90V A250D S255V Q475K T492N H494E |
| | | | 14 (UniProt: | A495E |
| | | | Q8GTB6) | |
| t826279 | 418 | 560 | SEQ ID NO: | R31Q HS6N I74T N90V A250P S255V Q475K T492N |
| | | | 14 (UniProt: | H494E A495E |
| | | | Q8GTB6) | |
| t825273 | 419 | 561 | SEQ ID NO: | R31Q K40Q H41Y V46P V52I H56N Q58S M61S I74T |
| | | | 14 (UniProt: | N90V V129I S255V V288L K296R F345L F360Y |
| | | | Q8GTB6) | A411V E424D Q475K T492N H494P A495E |
| t825101 | 420 | 562 | SEQ ID NO: | R31Q K40Q H41Y N44T A47T P49A L59F M61S I74T |
| | | | 14 (UniProt: | V85I S88L N90V A95G S255V T340E T492N H494E |
| | | | Q8GTB6) | A495E P542L H543R |
| t825049 | 421 | 563 | SEQ ID NO: | M61S N90V A250D S255V Q475K T492N A495E |
| | | | 14 (UniProt: | |
| | | | Q8GTB6) | |
| t826280 | 422 | 564 | SEQ ID NO: | I74T N90V A250D S255V E424D T492N A495E |
| | | | 14 (UniProt: | |
| | | | Q8GTB6) | |
| t824928 | 423 | 565 | SEQ ID NO: | R31Q K40Q H41Y I74T N90V S100A V129I H143E |
| | | | 14 (UniProt: | V288L K296R F345L T351I F360Y A411V E424D |
| | | | Q8GTB6) | T492N H494P A495E |
| t824526 | 424 | 566 | SEQ ID NO: | R31Q K40Q H41Y V46P H56N Q58S M61S I74T N90V |
| | | | 14 (UniProt: | V129I S255V V288L K296R F345L F360Y A411V |
| | | | Q8GTB6) | E424D T492N H494P A495E |
| t826284 | 425 | 567 | SEQ ID NO: | R31Q K40Q H41Y V52I M61S I74T N90V V129I |
| | | | 14 (UniProt: | S255V V288L K296R T340E F345L F360Y A411V |
| | | | Q8GTB6) | E424D Q475K T492N H494P A495E |
| t825914 | 426 | 568 | SEQ ID NO: | R31Q K40Q H41Y V46P V52I H56N M61S I74T N90V |
| | | | 14 (UniProt: | V129I S255V V288L K296R T340E F345L F360Y |
| | | | Q8GTB6) | A411V E424D Q475K T492N H494P A495E |
| t824903 | 427 | 569 | SEQ ID NO: | R31Q K40Q H41Y Q58S I74T N90V V129I H143E |
| | | | 14 (UniProt: | S255V V288L K296R F345L F360Y A411V E424D |
| | | | Q8GTB6) | T492N H494P A495E |
| t825013 | 428 | 570 | SEQ ID NO: | V46P M61S I74T N90V A250D S255V E424D Q475K |
| | | | 14 (UniProt: | T492N H494E A495E |
| | | | Q8GTB6) | |
| t826208 | 429 | 571 | SEQ ID NO: | V46P M61S I74T N90V A250P S255V T492N H494E |
| | | | 14 (UniProt: | |
| | | | Q8GTB6) | |
| t826237 | 430 | 572 | SEQ ID NO: | R31Q K40Q H41Y I74T N90V V129I S255V V288L |
| | | | 14 (UniProt: | K296R F345L T351I F360Y A411V E424D H494P |
| | | | Q8GTB6) | A495E |
| t825309 | 431 | 573 | SEQ ID NO: | R31Q K40Q H41Y V46P Q58S I74T N90V V129I |
| | | | 14 (UniProt: | S255V V288L K296R T340E F345L F360Y A411V |
| | | | Q8GTB6) | E424D Q475K T492N H494P A495E |
| t825708 | 432 | 574 | SEQ ID NO: | R31Q K40Q H41Y Q58S I74T N90V V129I S255V |
| | | | 14 (UniProt: | V288L K296R F345L F360Y A411V E424D Q475K |
| | | | Q8GTB6) | T492N H494P A495E |
| t825501 | 433 | 575 | SEQ ID NO: | R31Q K40Q H41Y I74T N90V V129I V288L K296R |
| | | | 14 (UniProt: | F345L F360Y A411V E424D H494P A495E S255V |
| | | | Q8GTB6) | |
| t825085 | 434 | 576 | SEQ ID NO: | R31Q K40Q H41Y H56N I74T N90V V129I V288L |
| | | | 14 (UniProt: | K296R F345L F360Y A411V E424D Q475K H494P |
| | | | Q8GTB6) | A495E |
| t825057 | 435 | 577 | SEQ ID NO: | R31Q M61S I74T N90V A250P S255V Q475K T492N |
| | | | 14 (UniProt: | H494E A495E |
| | | | Q8GTB6) | |
| t825496 | 436 | 578 | SEQ ID NO: | R31Q K40Q H41Y I74T N90V V129I S255V V288L |
| | | | 14 (UniProt: | K296R T340E F345L F360Y A411V E424D Q475K |
| | | | Q8GTB6) | T492N H494P A495E |
| t824647 | 437 | 579 | SEQ ID NO: | N90V A250D S255V Q475K T492N H494E A495E |
| | | | 14 (UniProt: | |
| | | | Q8GTB6) | |
| t826137 | 438 | 580 | SEQ ID NO: | M61S I74T N90V H143E A250P S255V Q475K T492N |
| | | | 14 (UniProt: | H494E A495E |
| | | | Q8GTB6) | |
| t826278 | 439 | 581 | SEQ ID NO: | R31Q V46P I74T N90V A250D S255V Q475K T492N |
| | | | 14 (UniProt: | H494E |
| | | | Q8GTB6) | |
| t825141 | 440 | 582 | SEQ ID NO: | R31Q K40Q H41Y Q58S M61S I74T N90V V129I |
| | | | 14 (UniProt: | H143E S255V V288L K296R T340E F345L F360Y |
| | | | Q8GTB6) | A411V E424D T492N H494P A495E |
| t825009 | 441 | 583 | SEQ ID NO: | N90V A250P S25SV Q475K T492N H494E |
| | | | 14 (UniProt: | |
| | | | Q8GTB6) | |
| t825086 | 442 | 584 | SEQ ID NO: | R31Q K40Q H41Y V46P H56N Q58S M61S I74T N90V |
| | | | 14 (UniProt: | V129I H143E S255V V288L K296R T340E F345L |
| | | | Q8GTB6) | Y354F F360Y A411V E424D Q475K T492N H494P |
| | | | | A495E |
| t825075 | 443 | 585 | SEQ ID NO: | R31Q K40Q H41Y V46P H56N M61S I74T N90V |
| | | | 14 (UniProt: | V129I S255V V288L K296R T340E F345L F360Y |
| | | | Q8GTB6) | A411V E424D T492N H494P A495E |
| t825725 | 444 | 586 | SEQ ID NO: | R31Q K40Q H41Y N44T V46P A47T P49A H56N |
| | | | 14 (UniProt: | Q58S L59F M61S I74T V85I S88L N90V A95G H143E |
| | | | Q8GTB6) | S255V Q475K T492N H494B A495E P542L H543R |
| t825090 | 445 | 587 | SEQ ID NO: | R31Q K40Q H41Y I74T D76N N90V V129I V158L |
| | | | 14 (UniProt: | V288L K296R T340E F345L Y354F F360Y A411V |
| | | | Q8GTB6) | E424D H494P A495E |
| t824861 | 446 | 588 | SEQ ID NO: | R31Q V46P M61S I74T N90V A250P S255V T492N |
| | | | 14 (UniProt: | H494E A495E |
| | | | Q8GTB6) | |
| t825007 | 447 | 589 | SEQ ID NO: | R31Q K40Q H41Y N44T A47T P49A L59F M61S I74T |
| | | | 14 (UniProt: | V85I S88L N90V A95G H143E S255V T340E E424D |
| | | | Q8GTB6) | T492N H494E A495E P542L H543R |
| t825084 | 448 | 590 | SEQ ID NO: | R31Q M61S I74T N90V A250P S255V T492N H494E |
| | | | 14 (UniProt: | |
| | | | Q8GTB6) | |
| t825029 | 449 | 591 | SEQ ID NO: | R31Q K40Q H41Y I74T N90V V129I V288L K296R |
| | | | 14 (UniProt: | F345L F360Y A411V E424D H494P A495E H267N |
| | | | Q8GTB6) | |
| t825015 | 450 | 592 | SEQ ID NO: | R31Q H56N M61S I74T N90V A250P S255V T492N |
| | | | 14 (UniProt: | H494E A495E |
| | | | Q8GTB6) | |
| t825031 | 451 | 593 | SEQ ID NO: | R31Q K40Q H41Y I74T N90V V129I H143E S255V |
| | | | 14 (UniProt: | V288L K296R F345L F360Y A411V E424D H494P |
| | | | Q8GTB6) | A495E |
| t825047 | 452 | 594 | SEQ ID NO: | R31Q K40Q H41Y I74T N89D N90V V129I H143E |
| | | | 14 (UniProt: | V288L K296R F345L F360Y A411V E424D H494P |
| | | | Q8GTB6) | A495E |
| t825633 | 453 | 595 | SEQ ID NO: | R31Q K40Q H41Y I74T N90V V129I V288L K296R |
| | | | 14 (UniProt: | F345L F360Y A411V E424D H494P A495E Y417V |
| | | | Q8GTB6) | |
| t825092 | 454 | 596 | SEQ ID NO: | R31Q K40Q H41Y I74T N90V V129I V288L K296R |
| | | | 14 (UniProt: | F345L F360Y A411V E424D H494P A495E K491M |
| | | | Q8GTB6) | |
| t825105 | 455 | 597 | SEQ ID NO: | R31Q K40Q H41Y N44T V46P A47T P49A H56N L59F |
| | | | 14 (UniProt: | M61S I74T V85I S88L N90V A95G S255V Q475K |
| | | | Q8GTB6) | T492N H494E A495E P542L H543R |
| t825625 | 456 | 598 | SEQ ID NO: | V46P H56N I74T N90V A250P S255V Q475K T492N |
| | | | 14 (UniProt: | H494E A495E |
| | | | Q8GTB6) | |
| t824663 | 457 | 59 | SEQ ID NO: | M61S I74T N90V A250P S255V Q475K T492N H494E |
| | | | 14 (UniProt: | |
| | | | Q8GTB6) | |
| t824937 | 458 | 600 | SEQ ID NO: | R31Q K40Q H41Y V46P V52I H56N Q58S M61S I74T |
| | | | 14 (UniProt: | N90V V129I H143E S255V V288L K296R F345L T351I |
| | | | Q8GTB6) | F360Y A411V E424D Q475K T492N H494P A495E |
| t825087 | 459 | 601 | SEQ ID NO: | R31Q K40Q H41Y N44T A47T P49A L59F I74T V85I |
| | | | 14 (UniProt: | S88L N90V A95G S255V T340E Q475K T492N H494E |
| | | | Q8GTB6) | A495E P542L H543R |
| t825078 | 460 | 602 | SEQ ID NO: | R31Q K40Q H41Y I74T N90V V129I S255V V288L |
| | | | 14 (UniProt: | K296R F345L F360Y A411V E424D T446I H494P |
| | | | Q8GTB6) | A495E |
| t825058 | 461 | 603 | SEQ ID NO: | R31Q I74T N90V A250D S255V |
| | | | 14 (UniProt: | |
| | | | Q8GTB6) | |
| t825024 | 462 | 604 | SEQ ID NO: | R31Q K40Q H41Y M61S I74T N90V V129I S255V |
| | | | 14 (UniProt: | V288L K296R F345L F360Y A411V E424D H494P |
| | | | Q8GTB6) | A495E |
| t825109 | 463 | 605 | SEQ ID NO: | R31Q K40Q H41Y V46P Q58S I74T N90V V129I |
| | | | 14 (UniProt: | S255V V288L K296R F345L F360Y A411V E424D |
| | | | Q8GTB6) | T492N H494P A495E |

TABLE 19
Amino Acid Substitutions in Terminal Synthases Described in Example 5

| | Nucleic | | | |
|---|---|---|---|---|
| | acid | Amino | | |
| Strain | SEQ | acid SEQ | Reference | Amino Acid Substitutions Relative to |
| ID | ID NO: | ID NO: | Sequence ID | Reference Sequence |
| 865859 | 403 | 545 | SEQ ID NO: 13 | G95A T339E Q343E A414V |
| | | | (UniProt: | |
| | | | A6P6V9) | |
| 865977 | 418 | 560 | SEQ ID NO: 14 | R31Q H56N I74T N90V A250P S255V |
| | | | (UniProt: | Q475K T492N H494E A495E |
| | | | Q8GTB6) | |
| 923976 | 954 | 698 | SEQ ID NO: 14 | R31Q H56N Q58S I74T N90V A250P |
| | | | (UniProt: | S255V V288L F345L Q475K T492N |
| | | | Q8GTB6) | |
| 923759 | 955 | 699 | SEQ ID NO: 14 | R31Q V52I H56N Q58S M61S I74T |
| | | | (UniProt: | N90V A250P S255V F345L Q475K |
| | | | Q8GTB6) | T492N |
| 923624 | 956 | 700 | SEQ ID NO: 14 | R31Q H56N I74T N90V H143E A250P |
| | | | (UniProt: | S255V Q475K T492N |
| | | | Q8GTB6) | |
| 923980 | 957 | 701 | SEQ ID NO: 14 | R31Q H56N I74T N90V A250P S255V |
| | | | (UniProt: | L443I Q475K T492N |
| | | | Q8GTB6) | |
| 923922 | 958 | 702 | SEQ ID NO: 14 | H56N M61S N90V A250D S255V |
| | | | (UniProt: | V288L Q475K T492N A495E |
| | | | Q8GTB6) | |
| 923890 | 959 | 703 | SEQ ID NO: 14 | R31Q H56N I74T N90V K215R A250P |
| | | | (UniProt: | S255V Q475K T492N |
| | | | Q8GTB6) | |
| 923616 | 960 | 704 | SEQ ID NO: 14 | R31Q P49A H56N Q58S M61S I74T |
| | | | (UniProt: | N90VH143E A250P S255V V288L |
| | | | Q8GTB6) | F345L Q475K T492N |
| 923954 | 961 | 705 | SEQ ID NO: 14 | R31Q A47T H56N I74T N90V A250P |
| | | | (UniProt: | S255V Q475K T492N |
| | | | Q8GTB6) | |
| 923894 | 962 | 706 | SEQ ID NO: 14 | M61S N90V A250D S255V Q475K |
| | | | (UniProt: | T492N A495E N498T |
| | | | Q8GTB6) | |
| 923972 | 963 | 707 | SEQ ID NO: 14 | R31Q H56N M61S I74T N89H N90V |
| | | | (UniProt: | S100A H136R E150Q N196K N211D |
| | | | Q8GTB6) | A250P S255V V288M F345M S382K |
| | | | | L443I Q475K T492N |
| 923680 | 964 | 708 | SEQ ID NO: 14 | R31Q H56N Q58S M61S I74T N90V |
| | | | (UniProt: | H143E A250P S255V V288L T340E |
| | | | Q8GTB6) | F345L E424D Q475K T492N |
| 923918 | 965 | 709 | SEQ ID NO: 14 | R31Q H56N I74T S88L N90V A250P |
| | | | (UniProt: | S255V Q475K T492N |
| | | | Q8GTB6) | |
| 924465 | 966 | 710 | SBQ ID NO: 14 | R31Q V52I H56N Q58S M61S I74T |
| | | | (UniProt: | N90V H143E A250P S255V V288L |
| | | | Q8GTB6) | F345L Q475K T492N |
| 924725 | 967 | 711 | SEQ ID NO: 14 | R31Q H56N Q58S M61S I74T N90V |
| | | | (UniProt: | H143E A250P S255V V288L T340E |
| | | | Q8GTB6) | F345L E424D Q475K T492N P542L |
| 924927 | 968 | 712 | SEQ ID NO: 14 | R31Q H56N Q58S M61S I74T N90V |
| | | | (UniProt: | H143E A250P S255V V288L F345L |
| | | | Q8GTB6) | A411V Q475K T492N |
| 923908 | 969 | 713 | SEQ ID NO: 14 | R31Q V52I H56N Q58S M61S I74T |
| | | | (UniProt: | N90V A250P S255V V288L F345L |
| | | | Q8GTB6) | Q475K T492N |
| 923613 | 970 | 714 | SEQ ID NO: 14 | R31Q K50L H56N I74T N90V A250P |
| | | | (UniProt: | S255V Q475K T492N |
| | | | Q8GTB6) | |
| 924717 | 971 | 715 | SEQ ID NO: 14 | R31Q A47T V52I H56N Q58S M61S |
| | | | (UniProt: | I74T N90V H143E A250P S255V |
| | | | Q8GTB6) | V288L T340E F345L Q475K T492N |
| 924309 | 972 | 716 | SEQ ID NO: 14 | R31Q H56N M61S I74T N89H N90V |
| | | | (UniProt: | S100A N196K N211D A250P S255V |
| | | | Q8GTB6) | I257R V288M F345M S382K L443I |
| | | | | Q475K T492N |
| 923795 | 973 | 717 | SEQ ID NO: 14 | R31H M61S N90V A250D S255V |
| | | | (UniProt: | Q475K T492N A495E |
| | | | Q8GTB6) | |
| 923880 | 974 | 718 | SEQ ID NO: 14 | H56N Q58S M61S I74T N90V H143E |
| | | | (UniProt: | A250D S255V V288L F345L Q475K |
| | | | Q8GTB6) | T492N A495E |
| 924364 | 975 | 719 | SEQ ID NO: 14 | R31Q H56N Q58S M61S I74T N90V |
| | | | (UniProt: | H143E A250P S255V V288L Q475K |
| | | | Q8GTB6) | T492N |
| 924612 | 976 | 720 | SEQ ID NO: 14 | R31Q H56N I74T N89H N90V N196K |
| | | | (UniProt: | N211D A250P S255V F345M S382K |
| | | | Q8GTB6) | L443I Q475K T492N |
| 924164 | 977 | 721 | SEQ ID NO: 14 | R31Q H56N M61S I74T N89H N90V |
| | | | (UniProt: | S100A E150Q N196K N211D A250P |
| | | | Q8GTB6) | S255V I257R V288M F345M S382K |
| | | | | L443I Q475K T492N |
| 924803 | 978 | 722 | SEQ ID NO: 14 | R31Q H56N M61S I74T N89H N90V |
| | | | (UniProt: | S100A E150Q N196K N211D A250P |
| | | | Q8GTB6) | S255V V288M F345M S382K L443I |
| | | | | Q475K T492N |
| 924559 | 979 | 723 | SEQ ID NO: 14 | R31Q H56N I74T N90V A250P S255V |
| | | | (UniProt: | V288L Q475K T492N |
| | | | Q8GTB6) | |
| 924468 | 980 | 724 | SEQ ID NO: 14 | R31Q A47T H56N Q58S M61S I74T |
| | | | (UniProt: | N90V H143E A250P S255V V288L |
| | | | Q8GTB6) | T340E F345L E424D Q475K T492N |
| 923884 | 981 | 725 | SEQ ID NO: 14 | A47T H56N Q588 M61S I74T N90V |
| | | | (UniProt: | A250D S255V V288L T340E F345L |
| | | | Q8GTB6) | Q475K T492N |
| 924276 | 982 | 726 | SEQ ID NO: 14 | A47T H56N Q58S M61S I74T N90V |
| | | | (UniProt: | A250D S255V F345L E424D Q475K |
| | | | Q8GTB6) | T492N |
| 924665 | 983 | 727 | SEQ ID NO: 14 | R31Q H56N Q58S M61S I74T N90V |
| | | | (UniProt: | V129I H143E A250P S255V V288L |
| | | | Q8GTB6) | F345L E424D Q475K T492N |
| 924691 | 984 | 728 | SEQ ID NO: 14 | R31Q H56N M61S I74T N89H N90V |
| | | | (UniProt: | S100A N196K N211D A250P S255V |
| | | | Q8GTB6) | V288M F345M S382K L443I Q475K |
| | | | | T492N |
| 924639 | 985 | 729 | SEQ ID NO: 14 | R31Q H56N Q58S M61S I74T N90V |
| | | | (UniProt: | V129I H143E A250P S255V V288L |
| | | | Q8GTB6) | T340E F345L Q475K T492N |
| 924512 | 986 | 730 | SEQ ID NO: 14 | H56N Q58S M61S I74T N90V A250D |
| | | | (UniProt: | S255V V288L Q475K T492N |
| | | | Q8GTB6) | |
| 924723 | 987 | 731 | SEQ ID NO: 14 | H56N M61S I74T N90V H143E |
| | | | (UniProt: | A250D S255V V288L F345L Q475K |
| | | | Q8GTB6) | T492N |
| 923916 | 988 | 732 | SEQ ID NO: 14 | R31Q H56N Q58S M61S I74T N90V |
| | | | (UniProt: | H143E A250P S255V V288L F345L |
| | | | Q8GTB6) | E424D Q475K T492N |
| 924428 | 989 | 733 | SEQ ID NO: 14 | R31Q A47T H56N Q58S M61S I74T |
| | | | (UniProt: | N90V A250P S255V V288L T340E |
| | | | Q8GTB6) | F345L Q475K T492N |
| 924472 | 990 | 734 | SEQ ID NO: 14 | R31Q A47T H56N Q58S M61S I74T |
| | | | (UniProt: | N90V H143E A250P S255V V288L |
| | | | Q8GTB6) | T340E F345L Q475K T492N |
| 924609 | 991 | 735 | SEQ ID NO: 14 | R31Q H56N M61S I74T N90V A250P |
| | | | (UniProt: | S255V V288L T340E F345L Q475K |
| | | | Q8GTB6) | T492N |
| 924657 | 992 | 736 | SEQ ID NO: 14 | R31Q H56N I74T N90V A250P S255V |
| | | | (UniProt: | V288M Q475K T492N |
| | | | Q8GTB6) | |
| 924226 | 993 | 737 | SEQ ID NO: 14 | R31Q H56N Q58S M61S I74T N90V |
| | | | (UniProt: | V129I A250P S255V F345L Q475K |
| | | | Q8GTB6) | T492N |
| 924306 | 994 | 738 | SEQ ID NO: 14 | R31Q H56N I74T N90V N196K A250P |
| | | | (UniProt: | S255V Q475K T492N |
| | | | Q8GTB6) | |
| 924695 | 995 | 739 | SEQ ID NO: 14 | R31Q H56N M61S I74T N90V A250P |
| | | | (UniProt: | S255V T340E F345L E424D Q475K |
| | | | Q8GTB6) | T492N |
| 924162 | 996 | 740 | SEQ ID NO: 14 | H56N Q58S M61S I74T N90V A250D |
| | | | (UniProt: | S255V V288L T340E F345L Q475K |
| | | | Q8GTB6) | T492N A495E |
| 924387 | 997 | 741 | SEQ ID NO: 14 | A47T H56N Q58S M61S I74T N90V |
| | | | (UniProt: | A250D S255V F345L Q475K T492N |
| | | | Q8GTB6) | |
| 924667 | 998 | 742 | SEQ ID NO: 14 | H56N Q58S M61S I74T N90V A250D |
| | | | (UniProt: | S255V T340E F345L E424D Q475K |
| | | | Q8GTB6) | T492N |
| 924425 | 999 | 743 | SEQ ID NO: 14 | R31Q H56N I74T N90V A250P S255V |
| | | | (UniProt: | F345M Q475K T492N |
| | | | Q8GTB6) | |
| 924373 | 1000 | 744 | SEQ ID NO: 14 | H56N M61S I74T N90V A250D |
| | | | (UniProt: | S255V T340E F345L Q475K T492N |
| | | | Q8GTB6) | |
| 924212 | 1001 | 745 | SEQ ID NO: 14 | H56N M61S I74T N90V A250D |
| | | | (UniProt: | S255V V288L F345L Q475K T492N |
| | | | Q8GTB6) | |
| 924635 | 1002 | 746 | SEQ ID NO: 14 | H56N Q58S M61S I74T N90V H143E |
| | | | (UniProt: | A250D S255V F345L Q475K T492N |
| | | | Q8GTB6) | |
| 923912 | 1003 | 747 | SEQ ID NO: 14 | R31Q A47T H56N Q58S M61S I74T |
| | | | (UniProt: | N90V H143E A250P S255V V288L |
| | | | Q8GTB6) | F345L Q475K T492N |
| 924304 | 1004 | 748 | SEQ ID NO: 14 | R31Q H56N Q58S M61S I74T N90V |
| | | | (UniProt: | H143E A250P S255V V288L F345L |
| | | | Q8GTB6) | A411V E424D Q475K T492N |
| 924028 | 1005 | 749 | SEQ ID NO: 14 | R31Q H56N M61S I74T N90V A250P |
| | | | (UniProt: | S255V V288L F345L Q475K T492N |
| | | | Q8GTB6) | |
| 924426 | 1006 | 750 | SEQ ID NO: 14 | R31Q A47T H56N Q58S M61S I74T |
| | | | (UniProt: | N90V V129I A250P S255V V288L |
| | | | Q8GTB6) | T340E F345L E424D Q475K T492N |
| 924160 | 1007 | 751 | SEQ ID NO: 14 | R31Q H56N Q58S M61S I74T N90V |
| | | | (UniProt: | H143E A250P S255V T340E F345L |
| | | | Q8GTB6) | Q475K T492N |
| 924424 | 1008 | 752 | SEQ ID NO: 14 | A47T H56N Q58S M61S I74T N90V |
| | | | (UniProt: | H143E A250D S255V F345L E424D |
| | | | Q8GTB6) | Q475K T492N |
| 924431 | 1009 | 753 | SEQ ID NO: 14 | H56N M61S N90V A250D S255V |
| | | | (UniProt: | V288L F345L Q475K T492N A495E |
| | | | Q8GTB6) | |
| 924518 | 1010 | 754 | SEQ ID NO: 14 | P49A H56N Q58S M61S I74T N90V |
| | | | (UniProt: | H143E A250D S255V V288L F345L |
| | | | Q8GTB6) | Q475K T492N |
| 923771 | 1011 | 755 | SEQ ID NO: 14 | R31Q H56N Q58S M61S I74T N90V |
| | | | (UniProt: | A250P S255V V288L F345L A411V |
| | | | Q8GTB6) | Q475K T492N |
| 923744 | 1012 | 756 | SEQ ID NO: 14 | R31Q H56N Q58S M61S I74T N90V |
| | | | (UniProt; | A250P S255V V288L E424D Q475K |
| | | | Q8GTB6) | T492N |
| 924673 | 1013 | 757 | SEQ ID NO: 14 | R31Q H56N M61S I74T N90V A250P |
| | | | (UniProt: | S255V V288L Q475K T492N |
| | | | Q8GTB6) | |
| 924398 | 1014 | 758 | SEQ ID NO: 14 | H56N Q58S M61S I74T N90V A250D |
| | | | (UniProt: | S255V V288L F345L E424D Q475K |
| | | | Q8GTB6) | T492N A495E |
| 923948 | 1015 | 759 | SEQ ID NO: 14 | H56N Q58S M61S I74T N90V A250D |
| | | | (UniProt: | S255V V288L F345L E424D Q475K |
| | | | Q8GTB6) | T492N |
| 924062 | 1016 | 760 | SEQ ID NO: 14 | R31Q H56N M61S I74T N90V A250P |
| | | | (UniProt: | S255V F345L E424D Q475K T492N |
| | | | Q8GTB6) | |
| 923854 | 1017 | 761 | SEQ ID NO: 14 | R31Q H56N Q58S M61S I74T N90V |
| | | | (UniProt: | A250P S255V V288L Q475K T492N |
| | | | Q8GTB6) | |
| 923866 | 1018 | 762 | SEQ ID NO: 14 | H56N Q58S M61S I74T N90V A250D |
| | | | (UniProt: | S255V V288L Q475K T492N A495E |
| | | | Q8GTB6) | |
| 924645 | 1019 | 763 | SEQ ID NO: 14 | H56N M61S N90V A250D S255V |
| | | | (UniProt: | V288L F345L Q475K T492N |
| | | | Q8GTB6) | |
| 924649 | 1020 | 764 | SEQ ID NO: 14 | H56N Q58S M61S I74T N90V H143E |
| | | | (UniProt: | A250D S255V F345L E424D Q475K |
| | | | Q8GTB6) | T492N A495E |
| 923862 | 1021 | 765 | SEQ ID NO: 14 | R31Q A47T H56N Q58S M61S I74T |
| | | | (UniProt: | N90V H143E A250P S255V T340E |
| | | | Q8GTB6) | F345L Q475K T492N |
| 924699 | 1022 | 766 | SEQ ID NO: 14 | R31Q H56N M61S I74T N90V H143E |
| | | | (UniProt: | A250P S255V T340E F345L Q475K |
| | | | Q8GTB6) | T492N |
| 924417 | 1023 | 767 | SEQ ID NO: 14 | H56N M61S I74T N90V H143E |
| | | | (UniProt: | A250D S255V F345L Q475K T492N |
| | | | Q8GTB6) | |
| 924158 | 1024 | 768 | SEQ ID NO: 14 | R31Q P49A H56N Q58S M61S I74T |
| | | | (UniProt: | N90V V129I H143E A250P S255V |
| | | | Q8GTB6) | T340E F345L E424D Q475K T492N |
| 923840 | 1025 | 769 | SEQ ID NO: 14 | R31Q P49A H56N Q58S M61S I74T |
| | | | (UniProt: | N90V H143E A250P S255V T340E |
| | | | Q8GTB6) | F345L E424D Q475K T492N |
| 923758 | 1026 | 770 | SEQ ID NO: 14 | H56N M61S I74T N90V A250D |
| | | | (UniProt: | S255V V288L F345L Q475K T492N |
| | | | Q8GTB6) | A495E |
| 924719 | 1027 | 771 | SEQ ID NO: 14 | R31Q P49A H56N Q588 M61S I74T |
| | | | (UniProt: | N90V H143E A250P S255V F345L |
| | | | Q8GTB6) | Q475K T492N |
| 924006 | 1028 | 772 | SEQ ID NO: 14 | H56N Q58S M61S I74T N90V A250D |
| | | | (UniProt: | S255V V288L F345L Q475K T492N |
| | | | Q8GTB6) | |
| 924412 | 1029 | 773 | SEQ ID NO: 14 | R31Q A47T H56N Q58S M61S I74T |
| | | | (UniProt: | N90V H143E A250P S255V T340E |
| | | | Q8GTB6) | F345L E424D Q475K T492N |
| 924030 | 1030 | 774 | SEQ ID NO: 14 | H56N M61S N90V A250D S255V |
| | | | (UniProt: | F345L E424D Q475K T492N A495E |
| | | | Q8GTB6) | |
| 924094 | 1031 | 775 | SEQ ID NO: 14 | R31Q H56N Q58S M61S I74T N90V |
| | | | (UniProt: | A250P S255V T340E Q475K T492N |
| | | | Q8GTB6) | |
| 924484 | 1032 | 776 | SEQ ID NO: 14 | R31Q A47T H56N Q58S M61S I74T |
| | | | (UniProt: | N90V V129I H143E A250P S255V |
| | | | Q8GTB6) | V288L F345L B424D Q475K T492N |
| 924715 | 1033 | 777 | SEQ ID NO: 14 | H56N M61S N89H N90V S100A |
| | | | (UniProt: | E150Q N196K N211D A250D S255V |
| | | | Q8GTB6) | F345M L443I Q475K T492N A495E |
| 924576 | 1034 | 778 | SEQ ID NO: 14 | R31Q P49A H56N Q58S M61S I74T |
| | | | (UniProt: | N90VH143B A250P S255V V288L |
| | | | Q8GTB6) | T340E F345L E424D Q475K T492N |
| 924332 | 1035 | 779 | SEQ ID NO: 14 | R31Q H56N Q58S M61S I74T N90V |
| | | | (UniProt: | A250P S255V Q475K T492N |
| | | | Q8GTB6) | |
| 924298 | 1036 | 780 | SEQ ID NO: 14 | R31Q P49A H56N Q58S M61S I74T |
| | | | (UniProt: | N90V A250P S255V Q475K T492N |
| | | | Q8GTB6) | |
| 924083 | 1037 | 781 | SEQ ID NO: 14 | R31Q H56N Q58S M61S I74T N90V |
| | | | (UniProt: | H143E A250P S255V V288L T340E |
| | | | Q8GTB6) | F345L A411V E424D Q475K T492N |
| 924847 | 1038 | 782 | SEQ ID NO: 14 | R31Q H56N M61S I74T N90V H143E |
| | | | (UniProt: | A250P S255V V288L F345L Q475K |
| | | | Q8GTB6) | T492N |
| 924322 | 1039 | 783 | SEQ ID NO: 14 | H56N Q58S M61S I74T N90V A250D |
| | | | (UniProt: | S255V V288L T340E F345L E424D |
| | | | Q8GTB6) | Q475K T492N |
| 923811 | 1040 | 784 | SEQ ID NO: 13 | G95A N196K T339E Q343E A414V |
| | | | (UniProt: | |
| | | | A6P6V9) | |
| 924707 | 1041 | 785 | SEQ ID NO: 14 | H56N Q588 M618 N90V A250D |
| | | | (UniProt: | S255V T340E F345L Q475K T492N |
| | | | Q8GTB6) | A495E |
| 924928 | 1042 | 786 | SEQ ID NO: 13 | G95A E150Q V162I C180G N196K |
| | | | (UniProt: | N211D N273H T339E Q343E A414V |
| | | | A6P6V9) | |
| 923848 | 1043 | 787 | SEQ ID NO: 14 | R31Q H56N I74T N90V A250P S255V |
| | | | (UniProt: | T340E F345L Q475K T492N |
| | | | Q8GTB6) | |
| 924266 | 1044 | 788 | SEQ ID NO: 14 | R31Q H56N Q58S M61S I74T N90V |
| | | | (UniProt: | H143E A250P S255V Q475K T492N |
| | | | Q8GTB6) | |
| 924661 | 1045 | 789 | SEQ ID NO: 14 | R31Q H56N M61S I74T N90V H143E |
| | | | (UniProt: | A250P S255V V288L F345L E424D |
| | | | Q8GTB6) | Q475K T492N |
| 924744 | 1046 | 790 | SEQ ID NO: 13 | G95A S116A T339E Q343E A414V |
| | | | (UniProt: | N527D |
| | | | A6P6V9) | |
| 924748 | 1047 | 791 | SEQ ID NO: 13 | G95A Y175F T339E Q343E A414V |
| | | | (UniProt: | |
| | | | A6P6V9) | |
| 924828 | 1048 | 792 | SEQ ID NO: 13 | G95A H213N T339E Q343E L344M |
| | | | (UniProt: | A414V N527D |
| | | | A6P6V9) | |
| 924492 | 1049 | 793 | SEQ ID NO: 14 | R31Q V52I H56N M61S I74T N90V |
| | | | (UniProt: | A250P S255V Q475K T492N |
| | | | Q8GTB6) | |
| 923695 | 1050 | 794 | SEQ ID NO: 13 | G95A S116A T339E Q343E L344M |
| | | | (UniProt; | A414V N527D |
| | | | A6P6V9) | |
| 924932 | 1051 | 795 | SEQ ID NO: 13 | S116A H213N T339E Q343E L344M |
| | | | (UniProt: | N527D |
| | | | A6P6V9) | |
| 924446 | 1052 | 796 | SEQ ID NO: 14 | R31Q P49A H56N Q58S M61S I74T |
| | | | (UniProt: | N90V A250P S255V V288L F345L |
| | | | Q8GTB6) | E424D Q475K T492N |
| 924348 | 1053 | 797 | SEQ ID NO: 14 | P49A H56N Q58S M61S I74T N90V |
| | | | (UniProt: | H143E A250D S255V T340E F345L |
| | | | Q8GTB6) | Q475K T492N |
| 923786 | 1054 | 798 | SEQ ID NO: 13 | G95A S116A H213N T339E Q343E |
| | | | (UniProt: | L344M A414V N527D |
| | | | A6P6V9) | |
| 924942 | 1055 | 799 | SEQ ID NO: 13 | G95A T339E Q343E Y353M A414V |
| | | | (UniProt: | |
| | | | A6P6V9) | |
| 923960 | 1056 | 800 | SEQ ID NO: 13 | K50N G95A V103H E150Q V162I |
| | | | (UniProt: | C180G N196K N211D H213N T339E |
| | | | A6P6V9) | Q343E L344M A414V |
| 924284 | 1057 | 801 | SEQ ID NO: 13 | G95A T339E Q343E A414V L442I |
| | | | (UniProt: | |
| | | | A6P6V9) | |
| 924502 | 1058 | 802 | SEQ ID NO: 14 | R31Q K36H H56N I74T N90V A250P |
| | | | (UniProt: | S255V Q475K T492N |
| | | | Q8GTB6) | |
| 923727 | 1059 | 803 | SEQ ID NO: 13 | G95A T339E Q343E F380Y A414V |
| | | | (UniProt: | |
| | | | A6P6V9) | |
| 924413 | 1060 | 804 | SEQ ID NO: 13 | K50N G95A H213N T339E Q343E |
| | | | (UniProt: | L344M A414V N527D |
| | | | A6P6V9) | |
| 924406 | 1061 | 805 | SEQ ID NO: 14 | M61S N90V N196K A250D S255V |
| | | | (UniProt: | Q475K T492N A495E |
| | | | Q8GTB6) | |
| 924843 | 1062 | 806 | SEQ ID NO: 13 | G95A L230I T339E Q343E A414V |
| | | | (UniProt: | |
| | | | A6P6V9) | |
| 924965 | 1063 | 807 | SEQ ID NO: 13 | K50N G95A E150Q V162I C180G |
| | | | (UniProt: | N196K N211D H213N T339E Q343E |
| | | | A6P6V9) | L344M A414V |
| 924864 | 1064 | 808 | SEQ ID NO: 13 | K50N G95A H213N T339E Q343E |
| | | | (UniProt: | L344M A414V C446T |
| | | | A6P6V9) | |
| 924940 | 1065 | 809 | SEQ ID NO: 13 | K50N G95A N196K H213N T339E |
| | | | (UniProt: | Q343E L344M A414V |
| | | | A6P6V9) | |
| 923653 | 1066 | 810 | SEQ ID NO: 13 | G95A S116A H213N T339E Q343E |
| | | | (UniProt: | L344M A414V |
| | | | A6P6V9) | |
| 923780 | 1067 | 811 | SEQ ID NO: 14 | R31Q H56N M61S I74T N90V A250P |
| | | | (UniProt: | S255V V288L F345L E424D Q475K |
| | | | Q8GTB6) | T492N |
| 923850 | 1068 | 812 | SEQ ID NO: 13 | S116A H213N T339E Q343E N527D |
| | | | (UniProt: | |
| | | | A6P6V9) | |
| 923957 | 1069 | 813 | SEQ ID NO: 13 | G95A T339E Q343E L344M A414V |
| | | | (UniProt: | N527D |
| | | | A6P6V9) | |
| 924836 | 1070 | 814 | SEQ ID NO: 13 | K50N H69Q G95A S100A H213N |
| | | | (UniProt: | T339E Q343E L344M A414V |
| | | | A6P6V9) | |
| 924642 | 1071 | 815 | SEQ ID NO: 13 | G95A N211D T339E Q343E A414V |
| | | | (UniProt: | |
| | | | A6P6V9) | |
| 924888 | 1072 | 816 | SEQ ID NO: 13 | K50N E150Q V162I C180G N196K |
| | | | (UniProt: | N211D H213N T339E Q343E L344M |
| | | | A6P6V9) | |
| 923910 | 1073 | 817 | SEQ ID NO: 13 | S116A T339E Q343E N527D |
| | | | (UniProt: | |
| | | | A6P6V9) | |
| 924618 | 1074 | 818 | SEQ ID NO: 13 | S116A T339E Q343E L344M N527D |
| | | | (UniProt: | |
| | | | A6P6V9) | |
| 923896 | 1075 | 819 | SEQ ID NO: 13 | K50N G95A V162I C180G N196K |
| | | | (UniProt: | N211D H213N T339E Q343EL344M |
| | | | A6P6V9) | A414V |
| 924760 | 1076 | 820 | SEQ ID NO: 13 | K50N G95A S100A H213N T339E |
| | | | (UniProt: | Q343E L344M A414V N527D |
| | | | A6P6V9) | |
| 923899 | 1077 | 821 | SEQ ID NO: 13 | K50N H69Q G95A S100A H213N |
| | | | (UniProt: | T339E Q343E L344M A414V N527D |
| | | | A6P6V9) | |
| 924436 | 1078 | 822 | SEQ ID NO: 14 | R31Q K36Q H56N I74T N90V A250P |
| | | | (UniProt: | S255V Q475K T492N |
| | | | Q8GTB6) | |
| 924890 | 1079 | 823 | SEQ ID NO: 13 | K50N V162I C180G N196K N211D |
| | | | (UniProt: | H213N T339E Q343E L344M |
| | | | A6P6V9) | |
| 924240 | 1080 | 824 | SEQ ID NO: 14 | R31Q H56N L59F I74T N90V A250P |
| | | | (UniProt: | S255V Q475K T492N |
| | | | Q8GTB6) | |
| 924108 | 1081 | 825 | SEQ ID NO: 13 | K50N E150Q V162I A172P C180G |
| | | | (UniProt: | N196K N211D H213N T339E Q343E |
| | | | A6P6V9) | L344M |
| 924586 | 1082 | 826 | SEQ ID NO: 14 | R31Q H56N I74T N90V A250P S255V |
| | | | (UniProt: | V288L F345L Q475K T492N |
| | | | Q8GTB6) | |
| 924114 | 1083 | 827 | SEQ ID NO: 13 | K50N S100A S116A H213N T339E |
| | | | (UniProt: | Q343E L344M N527D |
| | | | A6P6V9) | |
| 924051 | 1084 | 828 | SEQ ID NO: 13 | G95A H213N T339E Q343E A414V |
| | | | (UniProt: | N527D |
| | | | A6P6V9) | |
| 924765 | 1085 | 829 | SEQ ID NO: 13 | K50N G95A S100A E150Q V162I |
| | | | (UniProt: | C180G N196K N211D H213N S322E |
| | | | A6P6V9) | T339E Q343E L344M A414V E452T |
| | | | | I504Q |
| 924801 | 1086 | 830 | SEQ ID NO: 13 | K50N G95A E150Q H213N T339E |
| | | | (UniProt: | Q343E L344M A414V |
| | | | A6P6V9) | |
| 924264 | 1087 | 831 | SEQ ID NO: 13 | G95A T339E Q343E L344M A414V |
| | | | (UniProt: | |
| | | | A6P6V9) | |
| 923611 | 1088 | 832 | SEQ ID NO: 13 | K50N G95A S100A V103H E150Q |
| | | | (UniProt; | V162I C180G N196K N211D H213N |
| | | | A6P6V9) | S322E T339E Q343E L344M A414V |
| | | | | E452T I504Q |
| 924640 | 1089 | 833 | SEQ ID NO: 13 | K50N G95A N211D H213N T339E |
| | | | (UniProt: | Q343E L344M A414V |
| | | | A6P6V9) | |
| 924747 | 1090 | 834 | SEQ ID NO: 13 | G95A T339E Q343E Q376V A414V |
| | | | (UniProt: | |
| | | | A6P6V9) | |
| 924912 | 1091 | 835 | SEQ ID NO: 13 | K50N H69Q G95A H213N T339E |
| | | | (UniProt: | Q343E L344M A414V N527D |
| | | | A6P6V9) | |
| 924767 | 1092 | 836 | SEQ ID NO: 13 | V162I C180G N196K N211D N273H |
| | | | (UniProt: | T339E Q343E |
| | | | A6P6V9) | |
| 924524 | 1093 | 837 | SEQ ID NO: 14 | H56N Q58S M61S N90V A250D |
| | | | (UniProt: | S255V Q475K T492N |
| | | | Q8GTB6) | |
| 924381 | 1094 | 838 | SEQ ID NO: 13 | G95A E150Q T339E Q343E A414V |
| | | | (UniProt: | |
| | | | A6P6V9) | |
| 924946 | 1095 | 839 | SEQ ID NO: 13 | K50N G95A S116A H213N T339E |
| | | | (UniProt: | Q343E A414V |
| | | | A6P6V9) | |
| 924945 | 1096 | 840 | SEQ ID NO: 13 | K50N H69Q G95A S100A S116A |
| | | | (UniProt: | H213N T339E Q343B L344M A414V |
| | | | A6P6V9) | N527D |
| 924764 | 1097 | 841 | SEQ ID NO: 13 | G95A T339E Q343E Q376R A414V |
| | | | (UniProt; | |
| | | | A6P6V9) | |
| 924378 | 1098 | 842 | SEQ ID NO: 13 | K50N G95A H213N T339E Q343E |
| | | | (UniProt: | L344M A414V L415M |
| | | | A6P6V9) | |
| 865957 | 1099 | 843 | SEQ ID NO: 13 | G95A H213N Q343E A414V |
| | | | (UniProt: | |
| | | | A6P6V9) | |
| 924716 | 1100 | 844 | SEQ ID NO: 13 | K50N G95A V103H H213N T339E |
| | | | (UniProt: | Q343E L344M A414V |
| | | | A6P6V9) | |
| 924528 | 1101 | 845 | SEQ ID NO: 13 | G95A H213N T339E Q343E L344M |
| | | | (UniProt: | A414V |
| | | | A6P6V9) | |
| 923805 | 1102 | 846 | SEQ ID NO: 13 | K50N G95A Y175F H213N T339E |
| | | | (UniProt: | Q343E L344M A414V |
| | | | A6P6V9) | |
| 924856 | 1103 | 847 | SEQ ID NO: 13 | N211D T339E Q343E |
| | | | (UniProt: | |
| | | | A6P6V9) | |
| 923765 | 1104 | 848 | SEQ ID NO: 13 | G95A S100A T339E Q343E A414V |
| | | | (UniProt: | |
| | | | A6P6V9) | |
| 924112 | 1105 | 849 | SEQ ID NO: 13 | K50N N196K H213N T339E Q343E |
| | | | (UniProt: | L344M |
| | | | A6P6V9) | |
| 924253 | 1106 | 850 | SEQ ID NO: 13 | K50N G95A H213N T339E Q343E |
| | | | (UniProt; | L344M A414V N527D |
| | | | A6P6V9) | |
| 924246 | 1107 | 851 | SEQ ID NO: 13 | G95A T339E Q343E A414V H542M |
| | | | (UniProt: | |
| | | | A6P6V9) | |
| 923672 | 1108 | 852 | SEQ ID NO: 13 | K50N G95A H213N T290A T339E |
| | | | (UniProt: | Q343E L344M A414V |
| | | | A6P6V9) | |
| 924957 | 1109 | 853 | SEQ ID NO: 13 | K50N H213NL2301 T339E Q343E |
| | | | (UniProt: | L344M |
| | | | A6P6V9) | |
| 924497 | 1110 | 854 | SEQ ID NO: 13 | G95A A172P T339E Q343E A414V |
| | | | (UniProt: | |
| | | | A6P6V9) | |
| 923818 | 1111 | 855 | SEQ ID NO: 13 | K50N G95A H213N T339E Q343E |
| | | | (UniProt: | A414V N527D |
| | | | A6P6V9) | |
| 923807 | 1112 | 856 | SEQ ID NO: 13 | G95A T339E Q343E Q376T A414V |
| | | | (UniProt: | |
| | | | A6P6V9) | |
| 924909 | 1113 | 857 | SEQ ID NO: 13 | G95A T339E Q343E A414V A479S |
| | | | (UniProt: | |
| | | | A6P6V9) | |
| 923942 | 1114 | 858 | SEQ ID NO: 13 | S116A H213N T339E Q343E |
| | | | (UniProt: | |
| | | | A6P6V9) | |
| 924745 | 1115 | 859 | SEQ ID NO: 13 | K50N H89Q G95A H213N T339E |
| | | | (UniProt: | Q343E L344M A414V |
| | | | A6P6V9) | |
| 924131 | 1116 | 860 | SEQ ID NO: 13 | G95A T339E Q343E A414V L481Y |
| | | | (UniProt: | |
| | | | A6P6V9) | |
| 924110 | 1117 | 861 | SEQ ID NO: 13 | K50N H69Q S100A H213N T339E |
| | | | (UniProt: | Q343E L344M N527D |
| | | | A6P6V9) | |
| 924227 | 1118 | 862 | SEQ ID NO: 13 | K50N H69Q S100A H213N T339E |
| | | | (UniProt: | Q343E L344M |
| | | | A6P6V9) | |
| 924978 | 1119 | 863 | SEQ ID NO: 13 | K50N G95A H213N T339E Q343E |
| | | | (UniProt: | L344M A414V D492N N527D |
| | | | A6P6V9) | |
| 923657 | 1120 | 864 | SEQ ID NO: 13 | G95A V216L T339E Q343E A414V |
| | | | (UniProt: | |
| | | | A6P6V9) | |
| 924139 | 1121 | 865 | SEQ ID NO: 13 | K50N S116A T339E Q343E N527D |
| | | | (UniProt: | |
| | | | A6P6V9) | |
| 924688 | 1122 | 866 | SEQ ID NO: 13 | K50N G95A H213N T339E Q343E |
| | | | (UniProt: | L344M N377P A414V |
| | | | A6P6V9) | |
| 924400 | 1123 | 867 | SEQ ID NO: 13 | G95A T339E Q343E A414V L481V |
| | | | (UniProt: | |
| | | | A6P6V9) | |
| 923726 | 1124 | 868 | SEQ ID NO: 13 | N196K T339E Q343E |
| | | | (UniProt: | |
| | | | A6P6V9) | |
| 924898 | 1125 | 869 | SEQ ID NO: 13 | T339E Q343E F380Y |
| | | | (UniProt; | |
| | | | A6P6V9) | |
| 923697 | 1126 | 870 | SEQ ID NO: 13 | K50N G95A T339E Q343E A414V |
| | | | (UniProt: | N527D |
| | | | A6P6V9) | |
| 924969 | 1127 | 871 | SEQ ID NO: 13 | G95A T339E Q343E A414V Y418F |
| | | | (UniProt: | |
| | | | A6P6V9) | |
| 923723 | 1128 | 872 | SEQ ID NO: 13 | G95A S322E T339E Q343E A414V |
| | | | (UniProt: | |
| | | | A6P6V9) | |
| 924167 | 1129 | 873 | SEQ ID NO: 13 | K50N S116A H213N T339E Q343E |
| | | | (UniProt: | L344M N527D |
| | | | A6P6V9) | |
| 924660 | 1130 | 874 | SEQ ID NO: 13 | T339E Q343E L344M N527D |
| | | | (UniProt: | |
| | | | A6P6V9) | |
| 923675 | 1131 | 875 | SEQ ID NO: 13 | K50N G95A H213N T290M T339E |
| | | | (UniProt: | Q343E L344M A414V |
| | | | A6P6V9) | |
| 924766 | 1132 | 876 | SEQ ID NO: 13 | G95A T339E Q343E Q376S A414V |
| | | | (UniProt: | |
| | | | A6P6V9) | |
| 924261 | 1133 | 877 | SEQ ID NO: 13 | H213N T339E Q343E L344M N527D |
| | | | (UniProt: | |
| | | | A6P6V9) | |
| 924962 | 1134 | 878 | SEQ ID NO: 13 | G95A N273H T339E Q343E A414V |
| | | | (UniProt: | |
| | | | A6P6V9) | |
| 924743 | 1135 | 879 | SEQ ID NO: 13 | G95A T339E Q343E A414V L481M |
| | | | (UniProt: | |
| | | | A6P6V9) | |
| 924900 | 1136 | 880 | SEQ ID NO: 13 | K50N G95A H213N T339E Q343E |
| | | | (UniProt: | L344M F352Y A414V |
| | | | A6P6V9) | |
| 924384 | 1137 | 881 | SEQ ID NO: 13 | K50N G95A T339E Q343E A414V |
| | | | (UniProt: | |
| | | | A6P6V9) | |
| 924622 | 1138 | 882 | SEQ ID NO: 13 | K50N G95A H213N T339E Q343E |
| | | | (UniProt: | L344M A414V 1445V |
| | | | A6P6V9) | |
| 924604 | 1139 | 883 | SEQ ID NO: 13 | T339E Q343E L442I |
| | | | (UniProt: | |
| | | | A6P6V9) | |
| 924433 | 1140 | 884 | SEQ ID NO: 14 | R31Q H56N Q58S M61S I74T N90V |
| | | | (UniProt: | A250P S255V V288L F345L E424D |
| | | | Q8GTB6) | Q475K T492N |
| 924487 | 1141 | 885 | SEQ ID NO: 14 | R31Q A47T H56N Q58S M61S I74T |
| | | | (UniProt: | N90V A250P S255V T340E F345L |
| | | | Q8GTB6) | E424D Q475K T492N |
| 924548 | 1142 | 886 | SEQ ID NO: 14 | R31Q H56N Q58S M61S I74T N90V |
| | | | (UniProt: | H143E A250P S255V V288L T340E |
| | | | Q8GTB6) | F345L Q475K T492N |
| 924829 | 1143 | 887 | SEQ ID NO: 14 | R31Q H56N M61S I74T N90V A250P |
| | | | (UniProt: | S255V T340E F345L Q475K T492N |
| | | | Q8GTB6) | |
| 924835 | 1144 | 888 | SEQ ID NO: 14 | R31Q A47T H56N M61S I74T N90V |
| | | | (UniProt: | A250P S255V F345L Q475K T492N |
| | | | Q8GTB6) | |
| 924851 | 1145 | 889 | SEQ ID NO: 14 | R31Q H56N Q58S M61S I74T N90V |
| | | | (UniProt: | A250P S255V F345L E424D Q475K |
| | | | Q8GTB6) | T492N |
| 924555 | 1146 | 890 | SEQ ID NO: 14 | A47T H56N Q58S M61S I74T N90V |
| | | | (UniProt: | A250D S255V V288L F345L Q475K |
| | | | Q8GTB6) | T492N |
| 923609 | 1147 | 891 | SEQ ID NO: 14 | H56N Q588 M61S I74T N90V H143E |
| | | | (UniProt: | A250D S255V V288L F345L Q475K |
| | | | Q8GTB6) | T492N |
| 924552 | 1148 | 892 | SEQ ID NO: 14 | H56N Q58S M61S I74T N90V H143E |
| | | | (UniProt: | A250D S255V T340E F345L Q475K |
| | | | Q8GTB6) | T492N |
| 924683 | 1149 | 893 | SEQ ID NO: 14 | R31Q H56N Q58S M61S I74T N90V |
| | | | (UniProt: | A250P S255V F345L A411V Q475K |
| | | | Q8GTB6) | T492N |
| 924455 | 1150 | 894 | SEQ ID NO: 14 | H56N M61S N89H N90V S100A |
| | | | (UniProt: | E150Q N196K N211D A250D S255V |
| | | | Q8GTB6) | N274H F345M Q475K T492N A495E |
| 924587 | 1151 | 895 | SEQ ID NO: 14 | R31Q H56N Q58S M61S I74T N90V |
| | | | (UniProt: | H143E A250P S255V V288L F345L |
| | | | Q8GTB6) | Q475K T492N |
| 924580 | 1152 | 896 | SEQ ID NO: 14 | V52I H56N Q58S M61S I74T N90V |
| | | | (UniProt: | A250D S255V V288L F345L Q475K |
| | | | Q8GTB6) | T492N |
| 924811 | 1153 | 897 | SEQ ID NO: 14 | R31Q H56N M61S I74T N90V H143E |
| | | | (UniProt: | A250P S255V F345L Q475K T492N |
| | | | Q8GTB6) | |
| 924855 | 1154 | 898 | SEQ ID NO: 14 | R31Q H56N Q58S M61S I74T N90V |
| | | | (UniProt: | H143E A250P S255V F345L B424D |
| | | | Q8GTB6) | Q475K T492N |
| 924218 | 1155 | 899 | SEQ ID NO: 14 | R31Q P49A H56N Q58S M61S I74T |
| | | | (UniProt: | N90V A250P S255V V288L T340E |
| | | | Q8GTB6) | F345L E424D Q475K T492N |
| 924214 | 1156 | 900 | SEQ ID NO: 14 | R31Q V52I H56N M61S I74T N90V |
| | | | (UniProt: | A250P S255V F345L Q475K T492N |
| | | | Q8GTB6) | |
| 924459 | 1157 | 901 | SEQ ID NO: 14 | A47T H56N Q58S M61S I74T N90V |
| | | | (UniProt: | H143E A250D S255V V288L F345L |
| | | | Q8GTB6) | Q475K T492N |
| 924520 | 1158 | 902 | SEQ ID NO: 14 | R31Q V52I H56N Q58S M61S I74T |
| | | | (UniProt: | N90V H143E A250P S255V V288L |
| | | | Q8GTB6) | T340E F345L E424D Q475K T492N |
| 924346 | 1159 | 903 | SEQ ID NO: 14 | R31Q H56N Q58S M61S I74T N90V |
| | | | (UniProt: | A250P S255V F345L Q475K T492N |
| | | | Q8GTB6) | |
| 923784 | 1160 | 904 | SEQ ID NO: 14 | A47T H56N Q588 M61S I74T N90V |
| | | | (UniProt: | H143E A250D S255V T340E F345L |
| | | | Q8GTB6) | Q475K T492N |
| 924681 | 1161 | 905 | SEQ ID NO: 14 | H56N Q58S M61S N90V A250D |
| | | | (UniProt: | S255V V288L F345L Q475K T492N |
| | | | Q8GTB6) | A495E |
| 924585 | 1162 | 906 | SEQ ID NO: 14 | A47T H56N Q58S M61S I74T N90V |
| | | | (UniProt: | H143E A250D S255V F345L Q475K |
| | | | Q8GTB6) | T492N |
| 924522 | 1163 | 907 | SEQ ID NO: 14 | H56N Q58S M61S I74T N90V H143E |
| | | | (UniProt: | A250D S255V V288L T340E F345L |
| | | | Q8GTB6) | E424D Q475K T492N A495E |
| 924262 | 1164 | 908 | SEQ ID NO: 14 | H56N Q58S M61S N90V A250D |
| | | | (UniProt: | S255V T340E F345L Q475K T492N |
| | | | Q8GTB6) | |
| 924693 | 1165 | 909 | SEQ ID NO: 14 | R3IQ P49A H56N Q58S M61S I74T |
| | | | (UniProt: | N90V A250P S255V T340E F345L |
| | | | Q8GTB6) | E424D Q475K T492N |
| 924686 | 1166 | 910 | SEQ ID NO: 14 | R31Q H56N Q58S I74T N90V H143E |
| | | | (UniProt: | A250P S255V F345L Q475K T492N |
| | | | Q8GTB6) | |
| 924583 | 1167 | 911 | SEQ ID NO: 14 | R31Q H56N I74T N90V A250P S255V |
| | | | (UniProt: | F345L E424D Q475K T492N |
| | | | Q8GTB6) | |
| 924595 | 1168 | 912 | SEQ ID NO: 14 | H56N Q58S M61S I74T N90V H143E |
| | | | (UniProt: | A250D S255V V288L F345L E424D |
| | | | Q8GTB6) | Q475K T492N A495E |
| 924867 | 1169 | 913 | SEQ ID NO: 13 | K50N G95A H213N T339E Q343E |
| | | | (UniProt: | L344M A414V |
| | | | A6P6V9) | |
| 924407 | 1170 | 914 | SEQ ID NO: 13 | V90C G95A T339E Q343E A414V |
| | | | (UniProt: | |
| | | | A6P6V9) | |
| 924352 | 1171 | 915 | SEQ ID NO: 13 | G95A V103H. T339E Q343E A414V |
| | | | (UniProt: | |
| | | | A6P6V9) | |
| 923928 | 1172 | 916 | SEQ ID NO: 13 | K50N G95A H213N T339E Q343E |
| | | | (UniProt: | L344M A414V C446V |
| | | | A6P6V9) | |
| 923647 | 1173 | 917 | SEQ ID NO: 13 | G95A C180G T339E Q343E A414V |
| | | | (UniProt: | |
| | | | A6P6V9) | |
| 924808 | 1174 | 918 | SEQ ID NO: 13 | G95A T339E Q343E A414V F467Y |
| | | | (UniProt: | |
| | | | A6P6V9) | |
| 924500 | 1175 | 919 | SEQ ID NO: 13 | G95A T339E Q343E Q376A A414V |
| | | | (UniProt: | |
| | | | A6P6V9) | |
| 923917 | 1176 | 920 | SEQ ID NO: 13 | K50N G95A H213N L230I T339E |
| | | | (UniProt: | Q343E L344M A414V |
| | | | A6P6V9) | |
| 923694 | 1177 | 921 | SEQ ID NO: 13 | G95A T339E Q343E Y386F A414V |
| | | | (UniProt: | |
| | | | A6P6V9) | |
| 923924 | 1178 | 922 | SEQ ID NO: 13 | K50N G95A S100A H213N T339E |
| | | | (UniProt: | Q343E L344M A414V D492N N527D |
| | | | A6P6V9) | |
| 923610 | 1179 | 923 | SEQ ID NO: 13 | G95A T339E Q343E A414V Y416I |
| | | | (UniProt: | |
| | | | A6P6V9) | |
| 923851 | 1180 | 924 | SEQ ID NO: 13 | K50N G95A H213N T339E Q343E |
| | | | (UniProt: | L344M A414V L442I |
| | | | A6P6V9) | |
| 923679 | 1181 | 925 | SEQ ID NO: 13 | G95A V162I T339E Q343E A414V |
| | | | (UniProt: | |
| | | | A6P6V9) | |
| 923806 | 1182 | 926 | SEQ ID NO: 13 | K50N G95A L171F H213N T339E |
| | | | (UniProt: | Q343E L344M A414V |
| | | | A6P6V9) | |
| 923603 | 1183 | 927 | SEQ ID NO: 13 | G95A T339B Q343B Q376N A414V |
| | | | (UniProt: | |
| | | | A6P6V9) | |
| 923870 | 1184 | 928 | SEQ ID NO: 13 | G95A Q124M T339E Q343E A414V |
| | | | (UniProt: | |
| | | | A6P6V9) | |
| 923836 | 1185 | 929 | SEQ ID NO: 13 | K50N G95A H213N F292M T339E |
| | | | (UniProt: | Q343B L344M A414V |
| | | | A6P6V9) | |
| 923935 | 1186 | 930 | SEQ ID NO: 13 | K50N G95A H213N T339E Q343E |
| | | | (UniProt: | A414V |
| | | | A6P6V9) | |
| 923946 | 1187 | 931 | SEQ ID NO: 13 | K50N G95A H213N T339E Q343E |
| | | | (UniProt: | L344M A414V L481I |
| | | | A6P6V9) | |
| 923617 | 1188 | 932 | SEQ ID NO: 13 | P79G G95A T339E Q343E A414V |
| | | | (UniProt: | |
| | | | A6P6V9) | |
| 924979 | 1189 | 933 | SEQ ID NO: 13 | K50N G95A H213N T339E Q343E |
| | | | (UniProt: | L344M Y386F A414V |
| | | | A6P6V9) | |
| 924920 | 1190 | 934 | SEQ ID NO: 13 | K50N G95A H213N T339E Q343E |
| | | | (UniProt: | L344M N377F A414V |
| | | | A6P6V9) | |
| 924960 | 1191 | 935 | SEQ ID NO: 13 | K50N G95A H213N T339E Q343E |
| | | | (UniProt: | L344M A414V I504Q |
| | | | A6P6V9) | |
| 924684 | 1192 | 936 | SEQ ID NO: 13 | K50N G95A H213N T339E Q343E |
| | | | (UniProt: | L344M A414V L486V |
| | | | A6P6V9) | |
| 924908 | 1193 | 937 | SEQ ID NO: 13 | K50N G95A H213N T339E Q343E |
| | | | (UniProt: | L344M N377R A414V |
| | | | A6P6V9) | |
| 924652 | 1194 | 938 | SEQ ID NO: 13 | K50N G95A H184F H213N T339E |
| | | | (UniProt: | Q343B L344M A414V |
| | | | A6P6V9) | |
| 924778 | 1195 | 939 | SEQ ID NO: 13 | L230I T339E Q343E |
| | | | (UniProt: | |
| | | | A6P6V9) | |
| 924800 | 1196 | 940 | SEQ ID NO: 13 | G95A T339E Q343E |
| | | | (UniProt: | |
| | | | A6P6V9) | |
| 923978 | 1197 | 941 | SEQ ID NO: 13 | K50N T339E Q343E L344M N527D |
| | | | (UniProt: | |
| | | | A6P6V9) | |
| 924812 | 1198 | 942 | SEQ ID NO: 13 | T339E Q343E A479T |
| | | | (UniProt: | |
| | | | A6P6V9) | |
| 923953 | 1199 | 943 | SEQ ID NO: 13 | S116A T339E Q343E |
| | | | (UniProt: | |
| | | | A6P6V9) | |
| 923955 | 1200 | 944 | SEQ ID NO: 13 | K50N S116A T339E Q343E L344M |
| | | | (UniProt: | N527D |
| | | | A6P6V9) | |
| 923974 | 1201 | 945 | SEQ ID NO: 13 | K50N H213N T339E Q343E L344M |
| | | | (UniProt: | N527D |
| | | | A6P6V9) | |
| 924746 | 1202 | 946 | SEQ ID NO: 13 | V216L T339E Q343E |
| | | | (UniProt: | |
| | | | A6P6V9) | |
| 924910 | 1203 | 947 | SEQ ID NO: 13 | T339E Q343E Q376G |
| | | | (UniProt: | |
| | | | A6P6V9) | |
| 924922 | 1204 | 948 | SEQ ID NO: 13 | S100A T339B Q343B |
| | | | (UniProt: | |
| | | | A6P6V9) | |
| 924929 | 1205 | 949 | SEQ ID NO: 13 | T339E Q343E Q376P |
| | | | (UniProt: | |
| | | | A6P6V9) | |
| 924961 | 1206 | 950 | SEQ ID NO: 13 | T339E Q343E E452T |
| | | | (UniProt: | |
| | | | A6P6V9) | |
| 924972 | 1207 | 951 | SEQ ID NO: 13 | T339E Q343E Q376T |
| | | | (UniProt: | |
| | | | A6P6V9) | |
| 924973 | 1208 | 952 | SEQ ID NO: 13 | T339E Q343E 1445A |
| | | | (UniProt: | |
| | | | A6P6V9) | |
| 924977 | 1209 | 953 | SEQ ID NO: 13 | K50N T339E Q343E L344M |
| | | | (UniProt: | |
| | | | A6P6V9) | |

TABLE 20
Sequences of Terminal Synthases described in Example 1

| | N-terminal | N-terminal | C-terminal | C-terminal | Complete | Complete |
|---|---|---|---|---|---|---|
| Strain | signal peptide | signal peptide | signal peptide | signal peptide | Fusion | Fusion |
| ID | (nucleotide) | (amino acid) | (nucleotide) | (amino acid) | (nucleotide) | (amino acid) |
| 631188 | n/a | n/a | (SEQ ID | (SEQ ID | (SEQ ID | (SEQ ID |
| | | | NO: 19) | NO: 17) | NO: 687) | NO: 686) |
| 631189 | (SEQ ID | (SEQ ID | n/a | n/a | (SEQ ID | (SEQ ID |
| | NO: 695) | NO: 694) | | | NO: 689) | NO: 688) |
| 631190 | (SEQ ID | (SEQ ID | (SEQ ID | (SEQ ID | (SEQ ID | (SEQ ID |
| | NO: 18) | NO: 16) | NO: 19) | NO: 17) | NO: 691) | NO: 690) |
| 631191 | (SEQ ID | (SEQ ID | (SEQ ID | (SEQ ID | (SEQ ID | (SEQ ID |
| | NO: 609) | NO: 608) | NO: 607) | NO: 17) | NO: 639) | NO: 638) |
| 631192 | (SEQ ID | (SEQ ID | (SEQ ID | (SEQ ID | (SEQ ID | (SEQ ID |
| | NO: 609) | NO: 608) | NO: 631) | NO: 630) | NO: 641) | NO: 640) |
| 631193 | (SEQ ID | (SEQ ID | (SEQ ID | (SEQ ID | (SEQ ID | (SEQ ID |
| | NO: 35) | NO: 16) | NO: 607) | NO: 17) | NO: 32) | NO: 23) |
| 631194 | (SEQ ID | (SEQ ID | (SEQ ID | (SEQ ID | (SEQ ID | (SEQ ID |
| | NO: 35) | NO: 16) | NO: 631) | NO: 630) | NO: 643) | NO: 642) |
| 631195 | (SEQ ID | (SEQ ID | (SEQ ID | (SEQ ID | (SEQ ID | (SEQ ID |
| | NO: 611) | NO: 610) | NO: 607) | NO: 17) | NO: 645) | NO: 644) |
| 631196 | (SEQ ID | (SEQ ID | (SEQ ID | (SEQ ID | (SEQ ID | (SEQ ID |
| | NO: 611) | NO: 610) | NO: 631) | NO: 630) | NO: 647) | NO: 646) |
| 631197 | (SEQ ID | (SEQ ID | (SEQ ID | (SEQ ID | (SEQ ID | (SEQ ID |
| | NO: 613) | NO: 612) | NO: 607) | NO: 17) | NO: 649) | NO: 648) |
| 631198 | (SEQ ID | (SEQ ID | (SEQ ID | (SEQ ID | (SEQ ID | (SEQ ID |
| | NO: 613) | NO: 612) | NO: 631) | NO: 630) | NO: 651) | NO: 650) |
| 631199 | (SEQ ID | (SEQ ID | (SEQ ID | (SEQ ID | (SEQ ID | (SEQ ID |
| | NO: 615) | NO: 614) | NO: 607) | NO: 17) | NO: 653) | NO: 652) |
| 631200 | (SEQ ID | (SEQ ID | (SEQ ID | (SEQ ID | (SEQ ID | (SEQ ID |
| | NO: 615) | NO: 614) | NO: 631) | NO: 630) | NO: 655) | NO: 654) |
| 631201 | n/a | n/a | (SEQ ID | (SEQ ID | (SEQ ID | (SEQ ID |
| | | | NO: 633) | NO: 632) | NO: 657) | NO: 656) |
| 631202 | n/a | n/a | (SEQ ID | (SEQ ID | (SEQ ID | (SEQ ID |
| | | | NO: 635) | NO: 634) | NO: 659) | NO: 658) |
| 631203 | n/a | n/a | (SEQ ID | (SEQ ID | (SEQ ID | (SEQ ID |
| | | | NO: 637) | NO: 636) | NO: 661) | NO: 660) |
| 631204 | (SEQ ID | (SEQ ID | n/a | n/a | (SEQ ID | (SEQ ID |
| | NO: 617) | NO: 616) | | | NO: 663) | NO: 662) |
| 631205 | (SEQ ID | (SEQ ID | n/a | n/a | (SEQ ID | (SEQ ID |
| | NO: 619) | NO: 618) | | | NO: 665) | NO: 664) |
| 631206 | (SEQ ID | (SEQ ID | n/a | n/a | (SEQ ID | (SEQ ID |
| | NO: 609) | NO: 608) | | | NO: 667) | NO: 666) |
| 631207 | (SEQ ID | (SEQ ID | n/a | n/a | (SEQ ID | (SEQ ID |
| | NO: 35) | NO: 16) | | | NO: 669) | NO: 668) |
| 631208 | (SEQ ID | (SEQ ID | n/a | n/a | (SEQ ID | (SEQ ID |
| | NO: 615) | NO: 614) | | | NO: 671) | NO: 670) |
| 631209 | (SEQ ID | (SEQ ID | n/a | n/a | (SEQ ID | (SEQ ID |
| | NO: 613) | NO: 612) | | | NO: 673) | NO: 672) |
| 631210 | (SEQ ID | (SEQ ID | n/a | n/a | (SEQ ID | (SEQ ID |
| | NO: 621) | NO: 620) | | | NO: 675) | NO: 674) |
| 631211 | (SEQ ID | (SEQ ID | n/a | n/a | (SEQ ID | (SEQ ID |
| | NO: 623) | NO: 622) | | | NO: 677) | NO: 676) |
| 631212 | (SEQ ID | (SEQ ID | n/a | n/a | (SEQ ID | (SEQ ID |
| | NO: 611) | NO: 610) | | | NO: 679) | NO: 678) |
| 631213 | (SEQ ID | (SEQ ID | n/a | n/a | (SEQ ID | (SEQ ID |
| | NO: 625) | NO: 624) | | | NO: 681) | NO: 680) |
| 631214 | (SEQ ID | (SEQ ID | n/a | n/a | (SEQ ID | (SEQ ID |
| | NO: 627) | NO: 626) | | | NO: 683) | NO: 682) |
| 631215 | (SEQ ID | (SEQ ID | n/a | n/a | (SEQ ID | (SEQ ID |
| | NO: 629) | NO: 628) | | | NO: 685) | NO: 684) |
| 631216 | (SEQ ID | (SEQ ID | n/a | n/a | (SEQ ID | (SEQ ID |
| | NO: 18) | NO: 16) | | | NO: 693) | NO: 692) |

TABLE 21
Sequences of Terminal Synthases described in Example 2*

| Strain | Nucleotide | Amino Acid |
|---|---|---|
| ID | SEQ ID NO: | SEQ ID NO: |
| 752452 | 27 | 36 |
| 752426 | 28 | 37 |
| 752445 | 29 | 38 |
| 752436 | 30 | 39 |
| 752430 | 31 | 40 |
| 752456 | 33 | 42 |
| 752427 | 34 | 43 |

*For the library screen, the terminal synthase sequences were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

TABLE 22
Sequences of Terminal Synthases described in Example 3*

| Strain | Nucleotide | Amino Acid |
|---|---|---|
| ID | SEQ ID NO: | SEQ ID NO: |
| 616314 | 45 | 136 |
| 701909 | 46 | 137 |
| 701916 | 47 | 138 |
| 701917 | 48 | 139 |
| 701919 | 49 | 140 |
| 701934 | 50 | 141 |
| 701940 | 51 | 142 |
| 701964 | 52 | 143 |
| 701992 | 53 | 144 |
| 701998 | 54 | 145 |
| 702004 | 55 | 146 |
| 702008 | 56 | 147 |
| 702022 | 57 | 148 |
| 702056 | 58 | 149 |
| 702080 | 59 | 150 |
| 702105 | 60 | 151 |
| 702109 | 61 | 152 |
| 702115 | 62 | 153 |
| 702118 | 63 | 154 |
| 702123 | 64 | 155 |
| 702136 | 65 | 156 |
| 702147 | 66 | 157 |
| 702150 | 67 | 158 |
| 702155 | 68 | 159 |
| 702187 | 69 | 160 |
| 702201 | 70 | 161 |
| 702215 | 71 | 162 |
| 702257 | 72 | 163 |
| 702258 | 73 | 164 |
| 702261 | 74 | 165 |
| 702276 | 75 | 166 |
| 702278 | 76 | 167 |
| 702280 | 77 | 168 |
| 702288 | 78 | 169 |
| 702297 | 79 | 170 |
| 702304 | 80 | 171 |
| 702308 | 81 | 172 |
| 702315 | 82 | 173 |
| 702329 | 83 | 174 |
| 702338 | 84 | 175 |
| 702342 | 85 | 176 |
| 702346 | 86 | 177 |
| 702350 | 87 | 178 |
| 702370 | 88 | 179 |
| 702376 | 89 | 180 |
| 702396 | 90 | 181 |
| 702412 | 91 | 182 |
| 702462 | 92 | 183 |
| 702470 | 93 | 184 |
| 702485 | 94 | 185 |
| 702507 | 95 | 186 |
| 702513 | 96 | 187 |
| 702517 | 97 | 188 |
| 702531 | 98 | 189 |
| 702563 | 99 | 190 |
| 702571 | 100 | 191 |
| 702581 | 101 | 192 |
| 702585 | 102 | 193 |
| 702591 | 103 | 194 |
| 702595 | 104 | 195 |
| 702601 | 105 | 196 |
| 702603 | 106 | 197 |
| 702660 | 107 | 198 |
| 702688 | 108 | 199 |
| 702891 | 109 | 200 |
| 702948 | 110 | 201 |
| 703131 | 111 | 202 |
| 703178 | 112 | 203 |
| 703300 | 113 | 204 |
| 703306 | 114 | 205 |
| 703341 | 115 | 206 |
| 703452 | 116 | 207 |
| 703455 | 117 | 208 |
| 703459 | 118 | 209 |
| 703473 | 119 | 210 |
| 703482 | 120 | 211 |
| 703520 | 121 | 212 |
| 703524 | 122 | 213 |
| 703528 | 123 | 214 |
| 703584 | 124 | 215 |
| 703607 | 125 | 216 |
| 703611 | 126 | 217 |
| 703634 | 127 | 218 |
| 703638 | 128 | 219 |
| 703685 | 129 | 220 |
| 703699 | 130 | 221 |
| 703703 | 131 | 222 |
| 703707 | 132 | 223 |
| 703721 | 133 | 224 |
| 703738 | 134 | 225 |
| 616315 | 22 | 21 |
| 701870 | 254 | 284 |
| 701939 | 255 | 285 |
| 701954 | 256 | 286 |
| 701963 | 257 | 287 |
| 701977 | 258 | 288 |
| 701990 | 259 | 289 |
| 701996 | 260 | 290 |
| 702000 | 261 | 291 |
| 702043 | 262 | 292 |
| 702050 | 263 | 293 |
| 702054 | 264 | 294 |
| 702090 | 265 | 295 |
| 702154 | 266 | 296 |
| 702232 | 267 | 297 |
| 702240 | 268 | 298 |
| 702761 | 269 | 299 |
| 702767 | 270 | 300 |
| 702801 | 271 | 301 |
| 702894 | 272 | 302 |
| 702927 | 273 | 303 |
| 702942 | 274 | 304 |
| 702993 | 275 | 305 |
| 703174 | 276 | 306 |
| 703239 | 277 | 307 |
| 703256 | 278 | 308 |
| 703289 | 279 | 309 |
| 703637 | 280 | 310 |
| 703690 | 281 | 311 |
| 703722 | 282 | 312 |
| 703725 | 283 | 313 |

*For the library screen, the terminal synthase sequences were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

TABLE 23
Sequences of Terminal Synthases described in Example 4*

| Strain | Nucleotide SEQ | Amino Acid SEQ |
|---|---|---|
| ID | ID NO: | ID NO: |
| t825123 | 322 | 464 |
| t825215 | 323 | 465 |
| t825585 | 324 | 466 |
| t826070 | 325 | 467 |
| t826072 | 326 | 468 |
| t826076 | 327 | 469 |
| t826096 | 328 | 470 |
| t825125 | 329 | 471 |
| t825189 | 330 | 472 |
| t825217 | 331 | 473 |
| t825219 | 332 | 474 |
| t825377 | 333 | 475 |
| t826030 | 334 | 476 |
| t826036 | 335 | 477 |
| t824622 | 336 | 478 |
| t825119 | 337 | 479 |
| t825129 | 338 | 480 |
| t825151 | 339 | 481 |
| t825213 | 340 | 482 |
| t825221 | 341 | 483 |
| t825379 | 342 | 484 |
| t826054 | 343 | 485 |
| t826100 | 344 | 486 |
| t826132 | 345 | 487 |
| t824932 | 346 | 488 |
| t825910 | 347 | 489 |
| t825025 | 348 | 490 |
| t825621 | 349 | 491 |
| t824996 | 350 | 492 |
| t824498 | 351 | 493 |
| t825269 | 352 | 494 |
| t825259 | 353 | 495 |
| t825978 | 354 | 496 |
| t825043 | 355 | 497 |
| t825528 | 356 | 498 |
| t824930 | 357 | 499 |
| t825077 | 358 | 500 |
| t825023 | 359 | 501 |
| t825103 | 360 | 502 |
| t824618 | 361 | 503 |
| t825071 | 362 | 504 |
| t825059 | 363 | 505 |
| t825126 | 364 | 506 |
| t825766 | 365 | 507 |
| t825987 | 366 | 508 |
| t826274 | 367 | 509 |
| t825341 | 368 | 510 |
| t826093 | 369 | 511 |
| t825841 | 370 | 512 |
| t824918 | 371 | 513 |
| t825034 | 372 | 514 |
| t825593 | 373 | 515 |
| t825277 | 374 | 516 |
| t825833 | 375 | 517 |
| t825936 | 376 | 518 |
| t825933 | 377 | 519 |
| t824603 | 378 | 520 |
| t824539 | 379 | 521 |
| t824659 | 380 | 522 |
| t824773 | 381 | 523 |
| t825907 | 382 | 524 |
| t825930 | 383 | 525 |
| t826097 | 384 | 526 |
| t825889 | 385 | 527 |
| t824654 | 386 | 528 |
| t824571 | 387 | 529 |
| t825248 | 388 | 530 |
| t824807 | 389 | 531 |
| t825877 | 390 | 532 |
| t826099 | 391 | 533 |
| t824746 | 392 | 534 |
| t824612 | 393 | 535 |
| t824626 | 394 | 536 |
| t825154 | 395 | 537 |
| t824540 | 396 | 538 |
| t824786 | 397 | 539 |
| t824688 | 398 | 540 |
| t825012 | 399 | 541 |
| t824646 | 400 | 542 |
| t825862 | 401 | 543 |
| t824653 | 402 | 544 |
| t824625 | 403 | 545 |
| t824942 | 404 | 546 |
| t825773 | 405 | 547 |
| t825108 | 406 | 548 |
| t825301 | 407 | 549 |
| t825935 | 408 | 550 |
| t825739 | 409 | 551 |
| t825724 | 410 | 552 |
| t824845 | 411 | 553 |
| t825099 | 412 | 554 |
| t825297 | 413 | 555 |
| t824990 | 414 | 556 |
| t824771 | 415 | 557 |
| t825263 | 416 | 558 |
| t825286 | 417 | 559 |
| t826279 | 418 | 560 |
| t825273 | 419 | 561 |
| t825101 | 420 | 562 |
| t825049 | 421 | 563 |
| t826280 | 422 | 564 |
| t824928 | 423 | 565 |
| t824526 | 424 | 566 |
| t826284 | 425 | 567 |
| t825914 | 426 | 568 |
| t824903 | 427 | 569 |
| t825013 | 428 | 570 |
| t826208 | 429 | 571 |
| t826237 | 430 | 572 |
| t825309 | 431 | 573 |
| t825708 | 432 | 574 |
| t825501 | 433 | 575 |
| t825085 | 434 | 576 |
| t825057 | 435 | 577 |
| t825496 | 436 | 578 |
| t824647 | 437 | 579 |
| t826137 | 438 | 580 |
| t826278 | 439 | 581 |
| t825141 | 440 | 582 |
| t825009 | 441 | 583 |
| t825086 | 442 | 584 |
| t825075 | 443 | 585 |
| t825725 | 444 | 586 |
| t825090 | 445 | 587 |
| t824861 | 446 | 588 |
| t825007 | 447 | 589 |
| t825084 | 448 | 590 |
| t825029 | 449 | 591 |
| t825015 | 450 | 592 |
| t825031 | 451 | 593 |
| t825047 | 452 | 594 |
| t825633 | 453 | 595 |
| t825092 | 454 | 596 |
| t825105 | 455 | 597 |
| t825625 | 456 | 598 |
| t824663 | 457 | 599 |
| t824937 | 458 | 600 |
| t825087 | 459 | 601 |
| t825078 | 460 | 602 |
| t825058 | 461 | 603 |
| t825024 | 462 | 604 |
| t825109 | 463 | 605 |
| t807949 | 22 | 21 |
| t807973 | 45 | 136 |
| t820182 | 254 | 284 |

*For the library screen, the terminal synthase sequences were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

TABLE 24
Sequences of Terminal Synthases described in Example 5*

| Strain | Nucleotide | Amino Acid |
|---|---|---|
| ID | SEQ ID NO: | SEQ ID NO: |
| 876606 | 22 | 21 |
| 876607 | 45 | 136 |
| 865859 | 403 | 545 |
| 865977 | 418 | 560 |
| 923976 | 954 | 698 |
| 923759 | 955 | 699 |
| 923624 | 956 | 700 |
| 923980 | 957 | 701 |
| 923922 | 958 | 702 |
| 923890 | 959 | 703 |
| 923616 | 960 | 704 |
| 923954 | 961 | 705 |
| 923894 | 962 | 706 |
| 923972 | 963 | 707 |
| 923680 | 964 | 708 |
| 923918 | 965 | 709 |
| 924465 | 966 | 710 |
| 924725 | 967 | 711 |
| 924927 | 968 | 712 |
| 923908 | 969 | 713 |
| 923613 | 970 | 714 |
| 924717 | 971 | 715 |
| 924309 | 972 | 716 |
| 923795 | 973 | 717 |
| 923880 | 974 | 718 |
| 924364 | 975 | 719 |
| 924612 | 976 | 720 |
| 924164 | 977 | 721 |
| 924803 | 978 | 722 |
| 924559 | 979 | 723 |
| 924468 | 980 | 724 |
| 923884 | 981 | 725 |
| 924276 | 982 | 726 |
| 924665 | 983 | 727 |
| 924691 | 984 | 728 |
| 924639 | 985 | 729 |
| 924512 | 986 | 730 |
| 924723 | 987 | 731 |
| 923916 | 988 | 732 |
| 924428 | 989 | 733 |
| 924472 | 990 | 734 |
| 924609 | 991 | 735 |
| 924657 | 992 | 736 |
| 924226 | 993 | 737 |
| 924306 | 994 | 738 |
| 924695 | 995 | 739 |
| 924162 | 996 | 740 |
| 924387 | 997 | 741 |
| 924667 | 998 | 742 |
| 924425 | 999 | 743 |
| 924373 | 1000 | 744 |
| 924212 | 1001 | 745 |
| 924635 | 1002 | 746 |
| 923912 | 1003 | 747 |
| 924304 | 1004 | 748 |
| 924028 | 1005 | 749 |
| 924426 | 1006 | 750 |
| 924160 | 1007 | 751 |
| 924424 | 1008 | 752 |
| 924431 | 1009 | 753 |
| 924518 | 1010 | 754 |
| 923771 | 1011 | 755 |
| 923744 | 1012 | 756 |
| 924673 | 1013 | 757 |
| 924398 | 1014 | 758 |
| 923948 | 1015 | 759 |
| 924062 | 1016 | 760 |
| 923854 | 1017 | 761 |
| 923866 | 1018 | 762 |
| 924645 | 1019 | 763 |
| 924649 | 1020 | 764 |
| 923862 | 1021 | 765 |
| 924699 | 1022 | 766 |
| 924417 | 1023 | 767 |
| 924158 | 1024 | 768 |
| 923840 | 1025 | 769 |
| 923758 | 1026 | 770 |
| 924719 | 1027 | 771 |
| 924006 | 1028 | 772 |
| 924412 | 1029 | 773 |
| 924030 | 1030 | 774 |
| 924094 | 1031 | 775 |
| 924484 | 1032 | 776 |
| 924715 | 1033 | 777 |
| 924576 | 1034 | 778 |
| 924332 | 1035 | 779 |
| 924298 | 1036 | 780 |
| 924083 | 1037 | 781 |
| 924847 | 1038 | 782 |
| 924322 | 1039 | 783 |
| 923811 | 1040 | 784 |
| 924707 | 1041 | 785 |
| 924928 | 1042 | 786 |
| 923848 | 1043 | 787 |
| 924266 | 1044 | 788 |
| 924661 | 1045 | 789 |
| 924744 | 1046 | 790 |
| 924748 | 1047 | 791 |
| 924828 | 1048 | 792 |
| 924492 | 1049 | 793 |
| 923695 | 1050 | 794 |
| 924932 | 1051 | 795 |
| 924446 | 1052 | 796 |
| 924348 | 1053 | 797 |
| 923786 | 1054 | 798 |
| 924942 | 1055 | 799 |
| 923960 | 1056 | 800 |
| 924284 | 1057 | 801 |
| 924502 | 1058 | 802 |
| 923727 | 1059 | 803 |
| 924413 | 1060 | 804 |
| 924406 | 1061 | 805 |
| 924843 | 1062 | 806 |
| 924965 | 1063 | 807 |
| 924864 | 1064 | 808 |
| 924940 | 1065 | 809 |
| 923653 | 1066 | 810 |
| 923780 | 1067 | 811 |
| 923850 | 1068 | 812 |
| 923957 | 1069 | 813 |
| 924836 | 1070 | 814 |
| 924642 | 1071 | 815 |
| 924888 | 1072 | 816 |
| 923910 | 1073 | 817 |
| 924618 | 1074 | 818 |
| 923896 | 1075 | 819 |
| 924760 | 1076 | 820 |
| 923899 | 1077 | 821 |
| 924436 | 1078 | 822 |
| 924890 | 1079 | 823 |
| 924240 | 1080 | 824 |
| 924108 | 1081 | 825 |
| 924586 | 1082 | 826 |
| 924114 | 1083 | 827 |
| 924051 | 1084 | 828 |
| 924765 | 1085 | 829 |
| 924801 | 1086 | 830 |
| 924264 | 1087 | 831 |
| 923611 | 1088 | 832 |
| 924640 | 1089 | 833 |
| 924747 | 1090 | 834 |
| 924912 | 1091 | 835 |
| 924767 | 1092 | 836 |
| 924524 | 1093 | 837 |
| 924381 | 1094 | 838 |
| 924946 | 1095 | 839 |
| 924945 | 1096 | 840 |
| 924764 | 1097 | 841 |
| 924378 | 1098 | 842 |
| 865957 | 1099 | 843 |
| 924716 | 1100 | 844 |
| 924528 | 1101 | 845 |
| 923805 | 1102 | 846 |
| 924856 | 1103 | 847 |
| 923765 | 1104 | 848 |
| 924112 | 1105 | 849 |
| 924253 | 1106 | 850 |
| 924246 | 1107 | 851 |
| 923672 | 1108 | 852 |
| 924957 | 1109 | 853 |
| 924497 | 1110 | 854 |
| 923818 | 1111 | 855 |
| 923807 | 1112 | 856 |
| 924909 | 1113 | 857 |
| 923942 | 1114 | 858 |
| 924745 | 1115 | 859 |
| 924131 | 1116 | 860 |
| 924110 | 1117 | 861 |
| 924227 | 1118 | 862 |
| 924978 | 1119 | 863 |
| 923657 | 1120 | 864 |
| 924139 | 1121 | 865 |
| 924688 | 1122 | 866 |
| 924400 | 1123 | 867 |
| 923726 | 1124 | 868 |
| 924898 | 1125 | 869 |
| 923697 | 1126 | 870 |
| 924969 | 1127 | 871 |
| 923723 | 1128 | 872 |
| 924167 | 1129 | 873 |
| 924660 | 1130 | 874 |
| 923675 | 1131 | 875 |
| 924766 | 1132 | 876 |
| 924261 | 1133 | 877 |
| 924962 | 1134 | 878 |
| 924743 | 1135 | 879 |
| 924900 | 1136 | 880 |
| 924384 | 1137 | 881 |
| 924622 | 1138 | 882 |
| 924604 | 1139 | 883 |
| 924433 | 1140 | 884 |
| 924487 | 1141 | 885 |
| 924548 | 1142 | 886 |
| 924829 | 1143 | 887 |
| 924835 | 1144 | 888 |
| 924851 | 1145 | 889 |
| 924555 | 1146 | 890 |
| 923609 | 1147 | 891 |
| 924552 | 1148 | 892 |
| 924683 | 1149 | 893 |
| 924455 | 1150 | 894 |
| 924587 | 1151 | 895 |
| 924580 | 1152 | 896 |
| 924811 | 1153 | 897 |
| 924855 | 1154 | 898 |
| 924218 | 1155 | 899 |
| 924214 | 1156 | 900 |
| 924459 | 1157 | 901 |
| 924520 | 1158 | 902 |
| 924346 | 1159 | 903 |
| 923784 | 1160 | 904 |
| 924681 | 1161 | 905 |
| 924585 | 1162 | 906 |
| 924522 | 1163 | 907 |
| 924262 | 1164 | 908 |
| 924693 | 1165 | 909 |
| 924686 | 1166 | 910 |
| 924583 | 1167 | 911 |
| 924595 | 1168 | 912 |
| 924867 | 1169 | 913 |
| 924407 | 1170 | 914 |
| 924352 | 1171 | 915 |
| 923928 | 1172 | 916 |
| 923647 | 1173 | 917 |
| 924808 | 1174 | 918 |
| 924500 | 1175 | 919 |
| 923917 | 1176 | 920 |
| 923694 | 1177 | 921 |
| 923924 | 1178 | 922 |
| 923610 | 1179 | 923 |
| 923851 | 1180 | 924 |
| 923679 | 1181 | 925 |
| 923806 | 1182 | 926 |
| 923603 | 1183 | 927 |
| 923870 | 1184 | 928 |
| 923836 | 1185 | 929 |
| 923935 | 1186 | 930 |
| 923946 | 1187 | 931 |
| 923617 | 1188 | 932 |
| 924979 | 1189 | 933 |
| 924920 | 1190 | 934 |
| 924960 | 1191 | 935 |
| 924684 | 1192 | 936 |
| 924908 | 1193 | 937 |
| 924652 | 1194 | 938 |
| 924778 | 1195 | 939 |
| 924800 | 1196 | 940 |
| 923978 | 1197 | 941 |
| 924812 | 1198 | 942 |
| 923953 | 1199 | 943 |
| 923955 | 1200 | 944 |
| 923974 | 1201 | 945 |
| 924746 | 1202 | 946 |
| 924910 | 1203 | 947 |
| 924922 | 1204 | 948 |
| 924929 | 1205 | 949 |
| 924961 | 1206 | 950 |
| 924972 | 1207 | 951 |
| 924973 | 1208 | 952 |
| 924977 | 1209 | 953 |

*For the library screen, the terminal synthase sequences were expressed with an N-terminal MFalpha2 signal peptide (SEQ ID NO: 16) and a C-terminal HDEL signal peptide (SEQ ID NO: 17). A methionine residue was also added at the amino terminus of SEQ ID NO: 16.

TABLE 25
Additional Terminal Synthase Sequences

| | | N-terminal | C-terminal | Complete |
|---|---|---|---|---|
| Strain | TS (amino | signal peptide | signal peptide | Fusion |
| ID | acid) | (amino acid) | (amino acid) | (amino acid) |
| 703738 | (SEQ ID | (SEQ ID NO: 16) | (SEQ ID NO: 17) | (SEQ ID |
| | NO: 225) | | | NO: 1210) |
| 702376 | (SEQ ID | (SEQ ID NO: 16) | (SEQ ID NO: 17) | (SEQ ID |
| | NO: 180) | | | NO: 1211) |
| 702350 | (SEQ ID | (SEQ ID NO: 16) | (SEQ ID NO: 17) | (SEQ ID |
| | NO: 178) | | | NO: 1212) |
| 702601 | (SEQ ID | (SEQ ID NO: 16) | (SEQ ID NO: 17) | (SEQ ID |
| | NO: 196) | | | NO: 1213) |
| 702660 | (SEQ ID | (SEQ ID NO: 16) | (SEQ ID NO: 17) | (SEQ ID |
| | NO: 198) | | | NO: 251) |
| 702258 | (SEQ ID | (SEQ ID NO: 16) | (SEQ ID NO: 17) | (SEQ ID |
| | NO: 164) | | | NO: 1214) |

TABLE 26
Additional Terminal Synthase Sequences

| Strain | | TS with signal peptides |
|---|---|---|
| ID | TS (nucleotide) | (nucleotide) |
| 703738 | (SEQ ID NO: 134) | (SEQ ID NO: 1215) |
| 702376 | (SEQ ID NO: 89) | (SEQ ID NO: 1216) |
| 702350 | (SEQ ID NO: 87) | (SEQ ID NO: 1217) |
| 702601 | (SEQ ID NO: 105) | (SEQ ID NO: 1218) |
| 702660 | (SEQ ID NO: 107) | (SEQ ID NO: 244) |
| 702258 | (SEQ ID NO: 73) | (SEQ ID NO: 1219) |

It should be appreciated that sequences disclosed in this application may or may not contain signal sequences. The sequences disclosed in this application encompass versions with or without signal sequences. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

1. A host cell that comprises a heterologous polynucleotide encoding a terminal synthase (TS), wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14, and wherein the TS is capable of producing a THC-type cannabinoid.

2. The host cell of claim 1, wherein relative to the sequence of SEQ ID NO: 14, the TS further comprises an amino acid substitution at one or more residues corresponding to positions 31, 40, 41, 46, 49, 51, 56, 59, 61, 63, 74, 90, 96, 100, 103, 116, 143, 173, 196, 250, 257, 290, 296, 311, 354, 377, 378, 411, 417, 446, 494, 495, 528, 542, 543 and/or 544 in SEQ ID NO: 14.

3. The host cell of claim 1 or 2, wherein the TS is capable of producing more of a THC-type cannabinoid than a control TS, wherein the control TS comprises the sequence of SEQ ID NO: 284 or SEQ ID NO: 21.

4. A host cell that comprises a heterologous polynucleotide encoding a terminal synthase (TS), wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459,462, 464, 469, 479, 475, 491, 492, 494, 495, 499, 528, 542, 543, and/or 544 in SEQ ID NO: 14, wherein the TS does not comprise SEQ ID NO: 20, 21, 320 or 321, wherein the TS is capable of producing more of a THC-type cannabinoid than a control TS, and wherein the control TS comprises the sequence of SEQ ID NO: 284 or SEQ ID NO: 21.

5. The host cell of any one of claims 1-4, wherein the THC-type cannabinoid is tetrahydrocannabinolic acid (THCA) and/or tetrahydrocannabivarinic acid (THCVA).

6. The host cell of any one of claims 1-5, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a THC-type cannabinoid than a control TS, and wherein the control TS comprises the sequence of SEQ ID NO: 284 or SEQ ID NO: 21.

7. The host cell of any one of claims 1-6, wherein the TS is capable of producing at least 1, 2, 3, or 4-fold more of a THC-type cannabinoid than a control TS, and wherein the control TS comprises the sequence of SEQ ID NO: 284 or SEQ ID NO: 21.

8. The host cell of any one of claims 2-7, wherein the TS comprises:
(i) the amino acid Q at a residue corresponding to position 31 in SEQ ID NO: 14;
(ii) the amino acid H or Q at a residue corresponding to position 36 in SEQ ID NO: 14;
(iii) the amino acid E or Q at a residue corresponding to position 40 in SEQ ID NO: 14;
(iv) the amino acid Y at a residue corresponding to position 41 in SEQ ID NO: 14;
(v) the amino acid T at a residue corresponding to position 44 in SEQ ID NO: 14;
(vi) the amino acid A or P at a residue corresponding to position 46 in SEQ ID NO: 14;
(vii) the amino acid T at a residue corresponding to position 47 in SEQ ID NO: 14;
(viii) the amino acid A at a residue corresponding to position 49 in SEQ ID NO: 14;
(ix) the amino acid F at a residue corresponding to position 51 in SEQ ID NO: 14;
(x) the amino acid I at a residue corresponding to position 52 in SEQ ID NO: 14;
(xi) the amino acid N at a residue corresponding to position 56 in SEQ ID NO: 14,
(xii) the amino acid P or S at a residue corresponding to position 58 in SEQ ID NO: 14;
(xiii) the amino acid F at a residue corresponding to position 59 in SEQ ID NO: 14;
(xiv) the amino acid S at a residue corresponding to position 61 in SEQ ID NO: 14;
(xv) the amino acid L or V at a residue corresponding to position 63 in SEQ ID NO: 14;
(xvi) the amino acid T at a residue corresponding to position 74 in SEQ ID NO. 14;
(xvii) the amino acid N at a residue corresponding to position 76 in SEQ ID NO: 14;
(xviii) the amino acid I at a residue corresponding to position 85 in SEQ ID NO: 14;
(xix) the amino acid L at a residue corresponding to position 88 in SEQ ID NO: 14;
(xx) the amino acid D, E, or H at a residue corresponding to position 89 in SEQ ID NO: 14;
(xxi) the amino acid E or V at a residue corresponding to position 90 in SEQ ID NO: 14;
(xxii) the amino acid G at a residue corresponding to position 95 in SEQ ID NO: 14;
(xxiii) the amino acid S at a residue corresponding to position 96 in SEQ ID NO: 14;
(xxiv) the amino acid A at a residue corresponding to position 100 in SEQ ID NO: 14;
(xxv) the amino acid I at a residue corresponding to position 103 in SEQ ID NO: 14;
(xxvi) the amino acid S at a residue corresponding to position 116 in SEQ ID NO: 14;
(xxvii) the amino acid I at a residue corresponding to position 129 in SEQ ID NO: 14;
(xxviii) the amino acid R at a residue corresponding to position 136 in SEQ ID NO: 14;
(xxix) the amino acid E at a residue corresponding to position 143 in SEQ ID NO: 14;
(xxx) the amino acid Q at a residue corresponding to position 150 in SEQ ID NO: 14;
(xxxi) the amino acid L at a residue corresponding to position 158 in SEQ ID NO: 14;
(xxxii) the amino acid A at a residue corresponding to position 173 in SEQ ID NO: 14;
(xxxiii) the amino acid A at a residue corresponding to position 181 in SEQ ID NO: 14;
(xxxiv) the amino acid K at a residue corresponding to position 196 in SEQ ID NO: 14;
(xxxv) the amino acid D at a residue corresponding to position 211 in SEQ ID NO: 14;
(xxxvi) the amino acid S at a residue corresponding to position 237 in SEQ ID NO: 14;
(xxxvii) the amino acid V at a residue corresponding to position 242 in SEQ ID NO: 14;
(xxxviii) the amino acid R at a residue corresponding to position 247 in SEQ ID NO: 14;
(xxxix) the amino acid D or P at a residue corresponding to position 250 in SEQ ID NO: 14;
(xl) the amino acid V at a residue corresponding to position 255 in SEQ ID NO: 14;
(xli) the amino acid M or R at a residue corresponding to position 257 in SEQ ID NO: 14;
(xlii) the amino acid N at a residue corresponding to position 267 in SEQ ID NO: 14;
(xliii) the amino acid E at a residue corresponding to position 268 in SEQ ID NO: 14;
(xliv) the amino acid V at a residue corresponding to position 273 in SEQ ID NO: 14;
(xlv) the amino acid H at a residue corresponding to position 274 in SEQ ID NO: 14;
(xlvi) the amino acid L, M, or T at a residue corresponding to position 288 in SEQ ID NO: 14;
(xlvii) the amino acid F at a residue corresponding to position 290 in SEQ ID NO: 14;
(xlviii) the amino acid R at a residue corresponding to position 296 in SEQ ID NO: 14;
(xlix) the amino acid Q at a residue corresponding to position 302 in SEQ ID NO: 14;
(l) the amino acid I at a residue corresponding to position 309 in SEQ ID NO: 14;
(li) the amino acid S at a residue corresponding to position 311 in SEQ ID NO: 14;
(lii) the amino acid L at a residue corresponding to position 318 in SEQ ID NO: 14;
(liii) the amino acid Q at a residue corresponding to position 329 in SEQ ID NO: 14;
(liv) the amino acid E at a residue corresponding to position 340 in SEQ ID NO: 14;
(lv) the amino acid Q at a residue corresponding to position 344 in SEQ ID NO: 14;
(lvi) the amino acid L or M at a residue corresponding to position 345 in SEQ ID NO: 14;
(lvii) the amino acid I at a residue corresponding to position 351 in SEQ ID NO: 14;
(lviii) the amino acid F at a residue corresponding to position 354 in SEQ ID NO: 14;
(lix) the amino acid Y at a residue corresponding to position 360 in SEQ ID NO: 14;
(lx) the amino acid D at a residue corresponding to position 361 in SEQ ID NO: 14;
(lxi) the amino acid T at a residue corresponding to position 363 in SEQ ID NO: 14;
(lxii) the amino acid Q at a residue corresponding to position 377 in SEQ ID NO: 14;
(lxiii) the amino acid N at a residue corresponding to position 378 in SEQ ID NO: 14;
(lxiv) the amino acid A at a residue corresponding to position 379 in SEQ ID NO: 14;
(lxv) the amino acid K at a residue corresponding to position 382 in SEQ ID NO: 14;
(lxvi) the amino acid V at a residue corresponding to position 396 in SEQ ID NO: 14;
(lxvii) the amino acid V at a residue corresponding to position 411 in SEQ ID NO: 14;
(lxviii) the amino acid V at a residue corresponding to position 417 in SEQ ID NO: 14;
(lxix) the amino acid F at a residue corresponding to position 419 in SEQ ID NO: 14;
(lxx) the amino acid D at a residue corresponding to position 424 in SEQ ID NO: 14;
(lxxi) the amino acid I at a residue corresponding to position 443 in SEQ ID NO: 14;
(lxxii) the amino acid I at a residue corresponding to position 446 in SEQ ID NO: 14;
(lxxiii) the amino acid L at a residue corresponding to position 459 in SEQ ID NO: 14;
(lxxiv) the amino acid I at a residue corresponding to position 462 in SEQ ID NO: 14;
(lxxv) the amino acid N at a residue corresponding to position 464 in SEQ ID NO: 14;
(lxxvi) the amino acid M at a residue corresponding to position 469 in SEQ ID NO: 14;
(lxxvii) the amino acid K at a residue corresponding to position 475 in SEQ ID NO: 14;
(lxxviii) the amino acid M at a residue corresponding to position 479 in SEQ ID NO: 14;
(lxxix) the amino acid M at a residue corresponding to position 491 in SEQ ID NO: 14;
(lxxx) the amino acid N at a residue corresponding to position 492 in SEQ ID NO: 14;
(lxxxi) the amino acid D, E, F, or P at a residue corresponding to position 494 in SEQ ID NO: 14;
(lxxxii) the amino acid E or K at a residue corresponding to position 495 in SEQ ID NO: 14;
(lxxxiii) the amino acid Q at a residue corresponding to position 499 in SEQ ID NO: 14;
(lxxxiv) the amino acid D at a residue corresponding to position 528 in SEQ ID NO: 14;
(lxxxv) the amino acid L or R at a residue corresponding to position 542 in SEQ ID NO: 14;
(lxxxvi) the amino acid R at a residue corresponding to position 543 in SEQ ID NO: 14; and/or
(lxxxvii) the amino acid R at a residue corresponding to position 544 in SEQ ID NO: 14.

9. The host cell of any one of claims 1-7, wherein the TS comprises:
(i) the amino acid H or Q at a residue corresponding to position 36 in SEQ ID NO: 14;
(ii) the amino acid T at a residue corresponding to position 44 in SEQ ID NO: 14;
(iii) the amino acid T at a residue corresponding to position 47 in SEQ ID NO: 14,
(iv) the amino acid I at a residue corresponding to position 52 in SEQ ID NO: 14;
(v) the amino acid P or S at a residue corresponding to position 58 in SEQ ID NO: 14;
(vi) the amino acid I at a residue corresponding to position 85 in SEQ ID NO: 14,
(vii) the amino acid L at a residue corresponding to position 88 in SEQ ID NO: 14;
(viii) the amino acid D, E, or H at a residue corresponding to position 89 in SEQ ID NO: 14;
(ix) the amino acid G at a residue corresponding to position 95 in SEQ ID NO: 14;
(x) the amino acid I at a residue corresponding to position 129 in SEQ ID NO: 14;
(xi) the amino acid R at a residue corresponding to position 136 in SEQ ID NO: 14;
(xii) the amino acid Q at a residue corresponding to position 150 in SEQ ID NO: 14;
(xiii) the amino acid L at a residue corresponding to position 158 in SEQ ID NO: 14;
(xiv) the amino acid A at a residue corresponding to position 181 in SEQ ID NO: 14;
(xv) the amino acid D at a residue corresponding to position 211 in SEQ ID NO: 14;
(xvi) the amino acid S at a residue corresponding to position 237 in SEQ ID NO: 14;
(xvii) the amino acid V at a residue corresponding to position 242 in SEQ ID NO: 14;
(xviii) the amino acid R at a residue corresponding to position 247 in SEQ ID NO: 14;
(xix) the amino acid V at a residue corresponding to position 255 in SEQ ID NO: 14;
(xx) the amino acid N at a residue corresponding to position 267 in SEQ ID NO: 14;
(xxi) the amino acid E at a residue corresponding to position 268 in SEQ ID NO: 14;
(xxii) the amino acid V at a residue corresponding to position 273 in SEQ ID NO: 14;
(xxiii) the amino acid H at a residue corresponding to position 274 in SEQ ID NO: 14;
(xxiv) the amino acid L, M, or T at a residue corresponding to position 288 in SEQ ID NO: 14;
(xxv) the amino acid Q at a residue corresponding to position 302 in SEQ ID NO: 14;
(xxvi) the amino acid I at a residue corresponding to position 309 in SEQ ID NO: 14;
(xxvii) the amino acid L at a residue corresponding to position 318 in SEQ ID NO: 14;
(xxviii) the amino acid Q at a residue corresponding to position 329 in SEQ ID NO: 14;
(xxix) the amino acid E at a residue corresponding to position 340 in SEQ ID NO: 14;
(xxx) the amino acid Q at a residue corresponding to position 344 in SEQ ID NO: 14,
(xxxi) the amino acid L or M at a residue corresponding to position 345 in SEQ ID NO: 14;
(xxxii) the amino acid I at a residue corresponding to position 351 in SEQ ID NO: 14;
(xxxiii) the amino acid Y at a residue corresponding to position 360 in SEQ ID NO: 14;
(xxxiv) the amino acid D at a residue corresponding to position 361 in SEQ ID NO: 14;
(xxxv) the amino acid T at a residue corresponding to position 363 in SEQ ID NO: 14;
(xxxvi) the amino acid A at a residue corresponding to position 379 in SEQ ID NO: 14;
(xxxvii) the amino acid K at a residue corresponding to position 382 in SEQ ID NO: 14;
(xxxviii) the amino acid V at a residue corresponding to position 396 in SEQ ID NO: 14;
(xxxix) the amino acid F at a residue corresponding to position 419 in SEQ ID NO: 14;
(xl) the amino acid D at a residue corresponding to position 424 in SEQ ID NO: 14;
(xli) the amino acid I at a residue corresponding to position 443 in SEQ ID NO: 14;
(xlii) the amino acid L at a residue corresponding to position 459 in SEQ ID NO: 14;
(xliii) the amino acid I at a residue corresponding to position 462 in SEQ ID NO: 14;
(xliv) the amino acid N at a residue corresponding to position 464 in SEQ ID NO: 14;
(xlv) the amino acid M at a residue corresponding to position 469 in SEQ ID NO: 14;
(xlvi) the amino acid M at a residue corresponding to position 479 in SEQ ID NO: 14,
(xlvii) the amino acid M at a residue corresponding to position 491 in SEQ ID NO: 14;
(xlviii) the amino acid N at a residue corresponding to position 492 in SEQ ID NO: 14; and/or
(xlix) the amino acid Q at a residue corresponding to position 499 in SEQ ID NO: 14.

10. The host cell of any one of claims 2-8, wherein the TS comprises at least 2, 3, 4, 5, 6, 7, 8, 9, 10, 11, 12, 13, 14, 15, or 16 amino acid substitutions at residues corresponding to positions 31, 36, 40, 41, 44, 46, 47, 49, 51, 52, 56, 58, 59, 61, 63, 74, 76, 85, 88, 89, 90, 95, 96, 100, 103, 116,129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14.

11. The host cell of any one of claims 1-9, wherein the TS comprises at least 2, 3, 4, 5, 6, 7, 8, 9, 10, 11, 12, 13, 14, 15, or 16 amino acid substitutions at residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14.

12. The host cell of any one of claims 2-11, wherein the TS comprises relative to SEQ ID NO: 14:
(i) R31Q, H56N, Q58S, M61S, I74T, N90V, H143E, A250P, S255V, V288L, T340E, F345L, E424D, Q475K, T492N, and P542L;
(ii) R31Q, V52I, H56N, Q58S, M61S, I74T, N90V, A250P, S255V, V288L, F345L, Q475K, and T492N;
(iii) R31Q, A47T, V52I, H56N, Q58S, M61S, I74T, N90V, H143E, A250P, S255V, V288L, T340E, F345L, Q475K, and T492N;
(iv) H56N, Q58S, M61S, I74T, N90V, H143E, A250D, S255V, V288L, F345L, Q475K, T492N, and A495E;
(v) R31Q, H56N, Q58S, M61S, I74T, N90V, H143E, A250P, S255V, V288L, Q475K, and T492N;
(vi) R31Q, A47T, H56N, Q58S, M61S, I74T, N90V, H143E, A250P, S255V, V288L, T340E, F345L, E424D, Q475K, and T492N;
(vii) A47T, H56N, Q58S, M61S, I74T, N90V, A250D, S255V, F345L, E424D, Q475K, and T492N;
(viii) R31Q, A47T, H56N, Q58S, M61S, I74T, N90V, A250P, S255V, V288L, T340E, F345L, Q475K, and T492N;
(ix) R31Q, A47T, H56N, Q58S, M61S, I74T, N90V, H143E, A250P, S255V, V288L, T340E, F345L, Q475K, and T492N;
(x) A47T, H56N, Q58S, M61S, I74T, N90V, A250D, S255V, F345L, Q475K, and T492N;
(xi) R31Q, A47T, H56N, Q58S, M61S, I74T, N90V, H143E, A250P, S255V, T340E, F345L, Q475K, and T492N;
(xii) R31Q, H56N, Q58S, M61S, I74T, N90V, A250P, S255V, V288L, F345L, E424D, Q475K, and T492N;
(xiii) R31Q, A47T, H56N, Q58S, M61S, I74T, N90V, A250P, S255V, T340E, F345L, E424D, Q475K, and T492N;
(xiv) A47T, H56N, Q58S, M61S, I74T, N90V, A250D, S255V, V288L, F345L, Q475K, and T492N;
(xv) H56N, Q58S, M61S, I74T, N90V, H143E, A250D, S255V, V288L, F345L, Q475K, and T492N; or
(xvi) R31Q, V52I, H56N, M61S, I74T, N90V, A250P, S255V, F345L, Q475K, and T492N.

13. The host cell of any one of claims 1-11, wherein the TS comprises relative to SEQ ID NO: 14:
(i) M61S, N90V, A250D, S255V, Q475K, T492N, and A495E;
(ii) H56N, M61S, I74T, N90V, A250P, S255V, T492N, and H494E; or
(iii) R31Q, H56N, I74T, N90V, A250P, S255V, Q475K, T492N, H494E, and A495E.

14. The host cell of claim 13, wherein the TS comprises a sequence that is at least 90%, at least 95%, at least 97%, at least 98%, or at least 99% identical to any one of SEQ ID NOs: 505, 563, or 560.

15. The host cell of any one of claims 1-13, wherein the TS comprises a sequence that is at least 90%, at least 95%, at least 97%, at least 98%, or at least 99% identical to any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913.

16. The host cell of claim 15, wherein the TS comprises a sequence that is at least 90%, at least 95%, at least 97%, at least 98%, or at least 99% identical to any one of SEQ ID NOs: 711, 713, 715, 718, 719, 724, 726, 733, 734, 741, 765, 884, 885, 890, 891, and 900.

17. The host cell of claim 15, wherein the TS comprises the sequence of any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913.

18. A host cell that comprises a heterologous polynucleotide encoding a terminal synthase (TS), wherein the TS comprises:
(i) a sequence that is at least 97% identical to SEQ ID NO: 40;
(ii) a sequence that is at least 98% identical to any one of SEQ ID NO: 37, 39, and 42;
(iii) a sequence that is at least 99% identical to SEQ ID NO: 43; or
(iv) a sequence comprising SEQ ID NO: 38;
wherein the host cell is capable of producing a THC-type cannabinoid.

19. The host cell of claim 18, wherein the THC-type cannabinoid is tetrahydrocannabinolic acid (THCA) and/or tetrahydrocannabivarinic acid (THCVA).

20. The host cell of claim 18 or 19, wherein the TS is capable of producing more of a THC-type cannabinoid than a control TS, wherein the control TS comprises the sequence of SEQ ID NO: 284 or SEQ ID NO: 21.

21. The host cell of any one of claims 18-20, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a THC-type cannabinoid than a control TS, wherein the control TS comprises the sequence of SEQ ID NO: 284 or SEQ ID NO: 21.

22. The host cell of any one of claims 1-21, wherein the TS further comprises a first signal peptide.

23. The host cell of claim 22, wherein the first signal peptide comprises SEQ ID NO: 16 or a sequence that has no more than two amino acid substitutions, insertions, additions, or deletions relative to the sequence of SEQ ID NO: 16.

24. The host cell of claim 22 or 23, wherein the first signal peptide is located at the amino terminus of the TS.

25. The host cell of claim 23 or 24, wherein a methionine residue is added to the N-terminus of SEQ ID NO: 16.

26. The host cell of any one of claims 22-25, wherein the TS further comprises a second signal peptide.

27. The host cell of claim 26, wherein the second signal peptide comprises SEQ ID NO: 17 or a sequence that has no more than one amino acid substitution, insertion, addition, or deletion relative to the sequence of SEQ ID NO: 17.

28. The host cell of claim 26 or 27, wherein the second signal peptide is located at the carboxyl terminus of the TS.

29. The host cell of any one of claims 1-28, wherein the host cell further produces one or more of cannabidiolic acid (CBDA), cannabidivarinic acid (CBDVA), cannabichromenic acid (CBCA) and/or cannabichromevarinic acid (CBCVA).

30. The host cell of any one of claims 1-29, wherein the TS produces a higher ratio of THCA:CBDA, THCA:CBCA, THCVA:CBDVA and/or THCVA:CBCVA than a control TS.

31. The host cell of claim 30, wherein the control TS is a TS comprising the sequence of SEQ ID NO: 284 or SEQ ID NO: 21.

32. The host cell of any one of 1-31, wherein the TS has a higher product specificity for a THC-type cannabinoid than a control TS.

33. The host cell of claim 32, wherein the control TS is a TS comprising the sequence of SEQ ID NO: 284 or SEQ ID NO: 21.

34. A host cell that comprises a heterologous polynucleotide encoding a terminal synthase (TS), wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13, and wherein the TS is capable of producing a CBD-type cannabinoid.

35. The host cell of claim 34, wherein relative to the sequence of SEQ ID NO: 13, the TS further comprises an amino acid substitution at one or more residues corresponding to positions 31, 47, 49, 50, 56, 57, 58, 69, 89, 95, 100, 103, 116, 124, 143, 162, 167, 168, 171, 172, 175, 180, 196, 213, 250, 287, 343, 344, 376, 377, 378, 394, 410, 414, 415, 445, 490, 492, 517 and/or 542 in SEQ ID NO: 13.

36. The host cell of claim 34 or 35, wherein the TS is capable of producing more of a CBD-type cannabinoid than a control TS, and wherein the control TS comprises the sequence of SEQ ID NO: 136.

37. A host cell that comprises a heterologous polynucleotide encoding a terminal synthase (TS), wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168, 171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13, wherein the TS is capable of producing more of a CBD-type cannabinoid than a control TS, and wherein the control TS comprises the sequence of SEQ ID NO: 136.

38. The host cell of any one of claims 34-37, wherein the CBD-type cannabinoid is CBDA and/or CBDVA.

39. The host cell of any one of claims 34-38, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBD-type cannabinoid than a control TS, wherein the control TS comprises the sequence of SEQ ID NO: 136.

40. The host cell of any one of claims 34-39, wherein the TS is capable of producing at least 1, 2, 3, or 4-fold more of a CBD-type cannabinoid than a control TS, and wherein the control TS comprises the sequence of SEQ ID NO: 136.

41. The host cell of any one of claims 35-40, wherein the TS comprises:
(i) the amino acid Q at a residue corresponding to position 31 in SEQ ID NO: 13;
(ii) the amino acid A at a residue corresponding to position 47 in SEQ ID NO: 13;
(iii) the amino acid P at a residue corresponding to position 49 in SEQ ID NO: 13;
(iv) the amino acid N at a residue corresponding to position 50 in SEQ ID NO: 13;
(v) the amino acid H at a residue corresponding to position 56 in SEQ ID NO: 13;
(vi) the amino acid D at a residue corresponding to position 57 in SEQ ID NO: 13;
(vii) the amino acid Q at a residue corresponding to position 58 in SEQ ID NO: 13;
(viii) the amino acid R or Q at a residue corresponding to position 69 in SEQ ID NO: 13;
(ix) the amino acid G at a residue corresponding to position 79 in SEQ ID NO: 13;
(x) the amino acid N, D, E, Q, or R at a residue corresponding to position 89 in SEQ ID NO: 13;
(xi) the amino acid C at a residue corresponding to position 90 in SEQ ID NO: 13;
(xii) the amino acid A at a residue corresponding to position 95 in SEQ ID NO: 13;
(xiii) the amino acid A at a residue corresponding to position 100 in SEQ ID NO: 13;
(xiv) the amino acid H at a residue corresponding to position 103 in SEQ ID NO: 13;
(xv) the amino acid E at a residue corresponding to position 106 in SEQ ID NO: 13;
(xvi) the amino acid A or G at a residue corresponding to position 116 in SEQ ID NO: 13;
(xvii) the amino acid N or M at a residue corresponding to position 124 in SEQ ID NO: 13;
(xviii) the amino acid E at a residue corresponding to position 143 in SEQ ID NO: 13;
(xix) the amino acid Q at a residue corresponding to position 150 in SEQ ID NO: 13;
(xx) the amino acid I at a residue corresponding to position 162 in SEQ ID NO: 13;
(xxi) the amino acid S at a residue corresponding to position 166 in SEQ ID NO: 13;
(xxii) the amino acid K at a residue corresponding to position 167 in SEQ ID NO: 13;
(xxiii) the amino acid T at a residue corresponding to position 168 in SEQ ID NO: 13;
(xxiv) the amino acid F at a residue corresponding to position 171 in SEQ ID NO: 13;
(xxv) the amino acid P at a residue corresponding to position 172 in SEQ ID NO: 13;
(xxvi) the amino acid F at a residue corresponding to position 175 in SEQ ID NO: 13;
(xxvii) the amino acid G at a residue corresponding to position 180 in SEQ ID NO: 13;
(xxviii) the amino acid F at a residue corresponding to position 184 in SEQ ID NO: 13;
(xxix) the amino acid K at a residue corresponding to position 196 in SEQ ID NO: 13;
(xxx) the amino acid D at a residue corresponding to position 211 in SEQ ID NO: 13;
(xxxi) the amino acid N at a residue corresponding to position 213 in SEQ ID NO: 13;
(xxxii) the amino acid L at a residue corresponding to position 216 in SEQ ID NO: 13;
(xxxiii) the amino acid I at a residue corresponding to position 230 in SEQ ID NO: 13;
(xxxiv) the amino acid R at a residue corresponding to position 250 in SEQ ID NO: 13;
(xxxv) the amino acid L at a residue corresponding to position 263 in SEQ ID NO: 13;
(xxxvi) the amino acid H at a residue corresponding to position 273 in SEQ ID NO: 13;
(xxxvii) the amino acid P at a residue corresponding to position 283 in SEQ ID NO: 13;
(xxxviii) the amino acid T at a residue corresponding to position 287 in SEQ ID NO: 13;
(xxxix) the amino acid M or A at a residue corresponding to position 290 in SEQ ID NO: 13;
(xl) the amino acid M at a residue corresponding to position 292 in SEQ ID NO: 13;
(xli) the amino acid D or N at a residue corresponding to position 319 in SEQ ID NO: 13;
(xlii) the amino acid E at a residue corresponding to position 322 in SEQ ID NO: 13;
(xliii) the amino acid E at a residue corresponding to position 339 in SEQ ID NO: 13;
(xliv) the amino acid E at a residue corresponding to position 343 in SEQ ID NO: 13;
(xlv) the amino acid M at a residue corresponding to position 344 in SEQ ID NO: 13;
(xlvi) the amino acid M at a residue corresponding to position 353 in SEQ ID NO: 13;
(xlvii) the amino acid L, Y, A, G, N, P, R, S, T, or V at a residue corresponding to position 376 in SEQ ID NO: 13,
(xlviii) the amino acid F, P, or R at a residue corresponding to position 377 in SEQ ID NO: 13;
(xlix) the amino acid K, R, S, or T at a residue corresponding to position 378 in SEQ ID NO: 13;
(l) the amino acid Y at a residue corresponding to position 380 in SEQ ID NO: 13;
(li) the amino acid F at a residue corresponding to position 386 in SEQ ID NO: 13;
(lii) the amino acid E at a residue corresponding to position 394 in SEQ ID NO: 13;
(liii) the amino acid E or K at a residue corresponding to position 397 in SEQ ID NO: 13;
(liv) the amino acid E at a residue corresponding to position 407 in SEQ ID NO: 13;
(lv) the amino acid T or V at a residue corresponding to position 410 in SEQ ID NO: 13;
(lvi) the amino acid I, L, M, T, or V at a residue corresponding to position 414 in SEQ ID NO: 13,
(lvii) the amino acid M at a residue corresponding to position 415 in SEQ ID NO: 13;
(lviii) the amino acid F, I, or M at a residue corresponding to position 416 in SEQ ID NO: 13;
(lix) the amino acid F at a residue corresponding to position 418 in SEQ ID NO: 13;
(lx) the amino acid S or T at a residue corresponding to position 441 in SEQ ID NO: 13;
(lxi) the amino acid I at a residue corresponding to position 442 in SEQ ID NO: 13;
(lxii) the amino acid V or A at a residue corresponding to position 445 in SEQ ID NO: 13;
(lxiii) the amino acid T or V at a residue corresponding to position 446 in SEQ ID NO: 13;
(lxiv) the amino acid S at a residue corresponding to position 450 in SEQ ID NO: 13,
(lxv) the amino acid T at a residue corresponding to position 452 in SEQ ID NO: 13;
(lxvi) the amino acid A at a residue corresponding to position 454 in SEQ ID NO: 13;
(lxvii) the amino acid Y at a residue corresponding to position 467 in SEQ ID NO: 13;
(lxviii) the amino acid S or T at a residue corresponding to position 479 in SEQ ID NO: 13;
(lxix) the amino acid I, M, V, or Y at a residue corresponding to position 481 in SEQ ID NO: 13;
(lxx) the amino acid V at a residue corresponding to position 486 in SEQ ID NO: 13;
(lxxi) the amino acid T at a residue corresponding to position 490 in SEQ ID NO: 13;
(lxxii) the amino acid N at a residue corresponding to position 492 in SEQ ID NO: 13;
(lxxiii) the amino acid Q at a residue corresponding to position 504 in SEQ ID NO: 13;
(lxxiv) the amino acid N at a residue corresponding to position 512 in SEQ ID NO: 13;
(lxxv) the amino acid D at a residue corresponding to position 527 in SEQ ID NO: 13; and/or
(lxxvi) the amino acid M at a residue corresponding to position 542 in SEQ ID NO: 13.

42. The host cell of any one of claims 34-40, wherein the TS comprises:
(i) the amino acid G at a residue corresponding to position 79 in SEQ ID NO: 13;
(ii) the amino acid C at a residue corresponding to position 90 in SEQ ID NO: 13,
(iii) the amino acid E at a residue corresponding to position 106 in SEQ ID NO: 13;
(iv) the amino acid Q at a residue corresponding to position 150 in SEQ ID NO: 13;
(v) the amino acid S at a residue corresponding to position 166 in SEQ ID NO: 13;
(vi) the amino acid D at a residue corresponding to position 211 in SEQ ID NO: 13;
(vii) the amino acid N at a residue corresponding to position 213 in SEQ ID NO: 13,
(viii) the amino acid L at a residue corresponding to position 216 in SEQ ID NO: 13;
(ix) the amino acid I at a residue corresponding to position 230 in SEQ ID NO: 13,
(x) the amino acid L at a residue corresponding to position 263 in SEQ ID NO: 13;
(xi) the amino acid H at a residue corresponding to position 273 in SEQ ID NO: 13;
(xii) the amino acid P at a residue corresponding to position 283 in SEQ ID NO: 13;
(xiii) the amino acid T at a residue corresponding to position 287 in SEQ ID NO: 13;
(xiv) the amino acid M or A at a residue corresponding to position 290 in SEQ ID NO: 13;
(xv) the amino acid M at a residue corresponding to position 292 in SEQ ID NO: 13;
(xvi) the amino acid D or N at a residue corresponding to position 319 in SEQ ID NO: 13;
(xvii) the amino acid E at a residue corresponding to position 322 in SEQ ID NO: 13;
(xviii) the amino acid E at a residue corresponding to position 339 in SEQ ID NO: 13;
(xix) the amino acid E at a residue corresponding to position 343 in SEQ ID NO: 13;
(xx) the amino acid M at a residue corresponding to position 344 in SEQ ID NO: 13;
(xxi) the amino acid M at a residue corresponding to position 353 in SEQ ID NO: 13;
(xxii) the amino acid Y at a residue corresponding to position 380 in SEQ ID NO: 13;
(xxiii) the amino acid F at a residue corresponding to position 386 in SEQ ID NO: 13;
(xxiv) the amino acid E at a residue corresponding to position 394 in SEQ ID NO: 13;
(xxv) the amino acid E or K at a residue corresponding to position 397 in SEQ ID NO: 13;
(xxvi) the amino acid E at a residue corresponding to position 407 in SEQ ID NO: 13;
(xxvii) the amino acid F, I, or M at a residue corresponding to position 416 in SEQ ID NO: 13;
(xxviii) the amino acid F at a residue corresponding to position 418 in SEQ ID NO: 13;
(xxix) the amino acid S or T at a residue corresponding to position 441 in SEQ ID NO: 13;
(xxx) the amino acid I at a residue corresponding to position 442 in SEQ ID NO: 13;
(xxxi) the amino acid T or V at a residue corresponding to position 446 in SEQ ID NO: 13;
(xxxii) the amino acid S at a residue corresponding to position 450 in SEQ ID NO: 13;
(xxxiii) the amino acid S or T at a residue corresponding to position 479 in SEQ ID NO: 13;
(xxxiv) the amino acid I, M, V, or Y at a residue corresponding to position 481 in SEQ ID NO: 13;
(xxxv) the amino acid V at a residue corresponding to position 486 in SEQ ID NO: 13;
(xxxvi) the amino acid T at a residue corresponding to position 490 in SEQ ID NO: 13;
(xxxvii) the amino acid Q at a residue corresponding to position 504 in SEQ ID NO: 13; and/or
(xxxviii) the amino acid N at a residue corresponding to position 512 in SEQ ID NO: 13.

43. The host cell of any one of claims 35-41, wherein the TS comprises at least 2, 3, 4, 5, 6, 7, 8, 9, 10, 11, 12, 13, 14, 15, or 16 amino acid substitutions at residues corresponding to positions 31, 47, 49, 50, 56, 57, 58, 69, 79, 89, 90, 95, 100, 103, 106, 116, 124, 143, 150, 162, 166,167, 168, 171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13.

44. The host cell of any one of claims 34-42, wherein the TS comprises at least 2, 3, 4, 5, 6, 7, 8, 9, 10, 11, 12, 13, 14, 15, or 16 amino acid substitutions at residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13.

45. The host cell of any one of claims 35-43, wherein the TS comprises relative to SEQ ID NO: 13:
(i) K50N, G95A, N196K, H213N, T339E, Q343E, L344M, and A414V;
(ii) G95A, Y175F, T339E, Q343E, and A414V;
(iii) G95A, S116A, T339E, Q343E, A414V, and N527D;
(iv) G95A, E150Q, V162I, C180G, N196K, N21 ID, N273H, T339E, Q343E, and A414V;
(v) G95A, T339E, Q343E, Q376V, and A414V;
(vi) K50N, G95A, S100A, E150Q, V1621, C180G, N196K, N211D, H213N, S322E, T339E, Q343E, L344M, A414V, E452T, and I504Q;
(vii) G95A, N196K, T339E, Q343E, and A414V;
(viii) 50N, G95A, V103H, H213N, T339E, Q343E, L344M, and A414V;
(ix) G95A, T339E, Q343E, Q376R, and A414V; or
(x) K50N, H213N, L230I, T339E, Q343E, and L344M.

46. The host cell of any one of claims 35-43, wherein the TS comprises relative to SEQ ID NO: 13:
(i) K50N, H213N, L230I, T339E, Q343E, and L344M;
(ii) S100A, T339E, and Q343E;
(iii) T339E, Q343E, L344M, and N527D;
(iv) K50N, V162I, C180G, N196K, N211D, H213N, T339E, Q343E, and L344M;
(v) K50N, E150Q, V162I, C180G, N196K, N211D, H213N, T339E, Q343E, and L344M;
(vi) S116A, H213N, T339E, Q343E, L344M, and N527D;
(vii) N196K, T339E, and Q343E;
(viii) K50N, E150Q, V162I, A172P, C180G, N196K, N211D, H213N, T339E, Q343E, and L344M;
(ix) V216L, T339E, and Q343E;
(x) S116A, H213N, T339E, Q343E, and N527D;
(xi) S116A, T339E, Q343E, and N527D; or
(xii) T339E, Q343E, and Q376P.

47. The host cell of any one of claims 34-46, wherein the TS comprises a sequence that is at least 90%, at least 95%, at least 96%, at least 97%, at least 98%, or at least 99/o identical to any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948.

48. The host cell of claim 47, wherein the TS comprises a sequence that is at least 90%, at least 95%, at least 96%, at least 97%, at least 98%, or at least 99% identical to any one of SEQ ID NOs: 784, 786, 792, 804, 828, 801, 806, 830, 808, 813, 809, 800, 815, 816 836, 825, 791, 845, 823, and 820.

49. The host cell of claim 47, wherein the TS comprises a sequence that is at least 90%, at least 95%, at least 96%, at least 97%, at least 98%, or at least 99% identical to any one of SEQ ID NOs: 795, 812, 816, 817, 823, 825, 853, 868, 874, 946, 948, and 949.

50. The host cell of claim 47, wherein the TS comprises the sequence of any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948.

51. A host cell that comprises a heterologous polynucleotide encoding a terminal synthase (TS), wherein the TS comprises a sequence that is at least 98% identical to SEQ ID NO: 36, wherein the host cell is capable of producing a CBD-type cannabinoid.

52. The host cell of claim 51, wherein the CBD-type cannabinoid is CBDA and/or CBDVA.

53. The host cell of claim 51 or 52, wherein the TS is capable of producing more of a CBD-type cannabinoid than a control TS, wherein the control TS comprises the sequence of SEQ ID NO: 136.

54. The host cell of any one of claims 51-53, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBD-type cannabinoid than a control TS, wherein the control TS comprises the sequence of SEQ ID NO: 136.

55. The host cell of any one of claims 34-54, wherein the TS further comprises a first signal peptide.

56. The host cell of claim 55, wherein the first signal peptide comprises SEQ ID NO: 16 or a sequence that has no more than two amino acid substitutions, insertions, additions, or deletions relative to the sequence of SEQ ID NO: 16.

57. The host cell of claim 55 or 56, wherein the first signal peptide is located at the amino terminus of the TS.

58. The host cell of claim 56 or 57, wherein a methionine residue is added to the N-terminus of SEQ ID NO: 16.

59. The host cell of any one of claims 55-58, wherein the TS further comprises a second signal peptide.

60. The host cell of claim 59, wherein the second signal peptide comprises SEQ ID NO: 17 or a sequence that has no more than one amino acid substitution, insertion, addition, or deletion relative to the sequence of SEQ ID NO: 17.

61. The host cell of claim 59 or 60, wherein the second signal peptide is located at the carboxyl terminus of the TS.

62. The host cell of any one of claims 34-61, wherein the host cell further produces one or more of THCA, THCVA, CBCA and/or CBCVA.

63. The host cell of any one of claims 34-62, wherein the TS produces a higher ratio of CBDA:THCA, CBDA:CBCA, CBDVA: THCVA and/or CBCVA:THCVA than a control TS.

64. The host cell of claim 63, wherein the control TS is a TS comprising the sequence of SEQ ID NO: 136.

65. The host cell of any one of 34-64, wherein the TS has a higher product specificity for a CBD-type cannabinoid than a control TS.

66. The host cell of claim 65, wherein the control TS is a TS comprising the sequence of SEQ ID NO: 136.

67. A host cell that comprises a heterologous polynucleotide encoding a terminal synthase (TS), wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462,464, 465, 469, 475, 479, 489, 491, 492, 493, 494, 496, 516, 524, 528, 542, 543, and/or 544 in SEQ ID NO: 14, and wherein the TS is capable of producing a CBC-type cannabinoid.

68. The host cell of claim 67, wherein relative to the sequence of SEQ ID NO: 14, the TS further comprises an amino acid substitution at one or more residues corresponding to positions 31, 40, 46, 74, 90, 255, 288, 290, 318, and/or 495 in SEQ ID NO: 14.

69. The host cell of claim 67 or 68, wherein the TS is capable of producing more of a CBC-type cannabinoid than a control TS, and wherein the control TS comprises the sequence of SEQ ID NO: 21.

70. A host cell that comprises a heterologous polynucleotide encoding a terminal synthase (TS), wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO. 14, wherein the TS is capable of producing more of a CBC-type cannabinoid than a control TS, and wherein the control TS comprises the sequence of SEQ ID NO: 21.

71. The host cell of any one of claims 67-69, wherein the CBC-type cannabinoid is CBCA and/or CBCVA.

72. The host cell of any one of claims 67-71, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300% more of a CBC-type cannabinoid than a control TS, wherein the control TS comprises the sequence of SEQ ID NO: 21.

73. The host cell of any one of claims 67-72, wherein the TS is capable of producing at least 1, 2, 3, or 4-fold more of a CBC-type cannabinoid than a control TS, wherein the control TS comprises the sequence of SEQ ID NO: 21.

74. The host cell of any one of claims 68-73, wherein the TS comprises:
(i) the amino acid Q at a residue corresponding to position 31 in SEQ ID NO: 14;
(ii) the amino acid E at a residue corresponding to position 40 in SEQ ID NO: 14;
(iii) the amino acid Y at a residue corresponding to position 41 in SEQ ID NO: 14;
(iv) the amino acid P at a residue corresponding to position 46 in SEQ ID NO: 14,
(v) the amino acid T at a residue corresponding to position 47 in SEQ ID NO: 14;
(vi) the amino acid A at a residue corresponding to position 49 in SEQ ID NO: 14;
(vii) the amino acid F at a residue corresponding to position 51 in SEQ ID NO: 14;
(viii) the amino acid I at a residue corresponding to position 52 in SEQ ID NO: 14;
(ix) the amino acid N at a residue corresponding to position 56 in SEQ ID NO: 14;
(x) the amino acid S at a residue corresponding to position 58 in SEQ ID NO: 14;
(xi) the amino acid S at a residue corresponding to position 61 in SEQ ID NO: 14;
(xii) the amino acid V or L at a residue corresponding to position 63 in SEQ ID NO: 14;
(xiii) the amino acid T at a residue corresponding to position 74 in SEQ ID NO: 14;
(xiv) the amino acid V at a residue corresponding to position 90 in SEQ ID NO: 14;
(xv) the amino acid G at a residue corresponding to position 95 in SEQ ID NO: 14;
(xvi) the amino acid S at a residue corresponding to position 96 in SEQ ID NO: 14;
(xvii) the amino acid I at a residue corresponding to position 103 in SEQ ID NO: 14;
(xviii) the amino acid S at a residue corresponding to position 116 in SEQ ID NO: 14;
(xix) the amino acid I at a residue corresponding to position 129 in SEQ ID NO: 14;
(xx) the amino acid R at a residue corresponding to position 136 in SEQ ID NO: 14;
(xxi) the amino acid E at a residue corresponding to position 143 in SEQ ID NO: 14;
(xxii) the amino acid L at a residue corresponding to position 158 in SEQ ID NO: 14;
(xxiii) the amino acid A at a residue corresponding to position 173 in SEQ ID NO: 14;
(xxiv) the amino acid A at a residue corresponding to position 181 in SEQ ID NO: 14;
(xxv) the amino acid S at a residue corresponding to position 237 in SEQ ID NO: 14;
(xxvi) the amino acid V at a residue corresponding to position 242 in SEQ ID NO: 14;
(xxvii) the amino acid R at a residue corresponding to position 247 in SEQ ID NO: 14;
(xxviii) the amino acid V at a residue corresponding to position 255 in SEQ ID NO: 14;
(xxix) the amino acid M at a residue corresponding to position 257 in SEQ ID NO: 14;
(xxx) the amino acid E at a residue corresponding to position 268 in SEQ ID NO: 14;
(xxxi) the amino acid V at a residue corresponding to position 273 in SEQ ID NO: 14;
(xxxii) the amino acid L at a residue corresponding to position 288 in SEQ ID NO: 14;
(xxxiii) the amino acid F at a residue corresponding to position 290 in SEQ ID NO: 14;
(xxxiv) the amino acid R at a residue corresponding to position 296 in SEQ ID NO: 14;
(xxxv) the amino acid Q at a residue corresponding to position 302 in SEQ ID NO: 14;
(xxxvi) the amino acid I at a residue corresponding to position 309 in SEQ ID NO: 14;
(xxxvii) the amino acid S at a residue corresponding to position 311 in SEQ ID NO: 14;
(xxxviii) the amino acid L at a residue corresponding to position 318 in SEQ ID NO: 14;
(xxxix) the amino acid E at a residue corresponding to position 340 in SEQ ID NO: 14;
(xl) the amino acid Q at a residue corresponding to position 344 in SEQ ID NO: 14;
(xli) the amino acid L at a residue corresponding to position 345 in SEQ ID NO: 14;
(xlii) the amino acid I at a residue corresponding to position 351 in SEQ ID NO: 14;
(xliii) the amino acid F at a residue corresponding to position 354 in SEQ ID NO: 14;
(xliv) the amino acid Y at a residue corresponding to position 360 in SEQ ID NO: 14;
(xlv) the amino acid D at a residue corresponding to position 361 in SEQ ID NO: 14;
(xlvi) the amino acid T at a residue corresponding to position 363 in SEQ ID NO: 14;
(xlvii) the amino acid Q at a residue corresponding to position 377 in SEQ ID NO: 14;
(xlviii) the amino acid N at a residue corresponding to position 378 in SEQ ID NO: 14;
(xlix) the amino acid A at a residue corresponding to position 379 in SEQ ID NO: 14;
(l) the amino acid S at a residue corresponding to position 382 in SEQ ID NO: 14;
(li) the amino acid V at a residue corresponding to position 396 in SEQ ID NO: 14;
(lii) the amino acid V at a residue corresponding to position 411 in SEQ ID NO: 14;
(liii) the amino acid D at a residue corresponding to position 424 in SEQ ID NO: 14;
(liv) the amino acid K at a residue corresponding to position 425 in SEQ ID NO: 14;
(lv) the amino acid T at a residue corresponding to position 430 in SEQ ID NO: 14;
(lvi) the amino acid I at a residue corresponding to position 442 in SEQ ID NO: 14;
(lvii) the amino acid I or V at a residue corresponding to position 443 in SEQ ID NO: 14;
(lviii) the amino acid I at a residue corresponding to position 446 in SEQ ID NO: 14;
(lix) the amino acid C at a residue corresponding to position 447 in SEQ ID NO: 14;
(lx) the amino acid L at a residue corresponding to position 459 in SEQ ID NO: 14;
(lxi) the amino acid I at a residue corresponding to position 462 in SEQ ID NO: 14;
(lxii) the amino acid N at a residue corresponding to position 464 in SEQ ID NO: 14;
(lxiii) the amino acid I at a residue corresponding to position 465 in SEQ ID NO: 14;
(lxiv) the amino acid M at a residue corresponding to position 469 in SEQ ID NO: 14;
(lxv) the amino acid K at a residue corresponding to position 475 in SEQ ID NO: 14;
(lxvi) the amino acid M at a residue corresponding to position 479 in SEQ ID NO: 14;
(lxvii) the amino acid I at a residue corresponding to position 489 in SEQ ID NO: 14;
(lxviii) the amino acid I at a residue corresponding to position 491 in SEQ ID NO: 14;
(lxix) the amino acid N at a residue corresponding to position 492 in SEQ ID NO: 14;
(lxx) the amino acid D at a residue corresponding to position 493 in SEQ ID NO: 14;
(lxxi) the amino acid F or P at a residue corresponding to position 494 in SEQ ID NO: 14;
(lxxii) the amino acid E or K at a residue corresponding to position 495 in SEQ ID NO: 14;
(lxxiii) the amino acid N at a residue corresponding to position 496 in SEQ ID NO: 14;
(lxxiv) the amino acid D at a residue corresponding to position 516 in SEQ ID NO: 14;
(lxxv) the amino acid L at a residue corresponding to position 524 in SEQ ID NO: 14;
(lxxvi) the amino acid D at a residue corresponding to position 528 in SEQ ID NO: 14;
(lxxvii) the amino acid L or R at a residue corresponding to position 542 in SEQ ID NO: 14;
(lxxviii) the amino acid R at a residue corresponding to position 543 in SEQ ID NO: 14; and/or
(lxxix) the amino acid R at a residue corresponding to position 544 in SEQ ID NO: 14.

75. The host cell of any one of claims 67-73, wherein the TS comprises:
(i) the amino acid Y at a residue corresponding to position 41 in SEQ ID NO: 14;
(ii) the amino acid T at a residue corresponding to position 47 in SEQ ID NO: 14;
(iii) the amino acid A at a residue corresponding to position 49 in SEQ ID NO: 14;
(iv) the amino acid F at a residue corresponding to position 51 in SEQ ID NO: 14,
(v) the amino acid I at a residue corresponding to position 52 in SEQ ID NO: 14;
(vi) the amino acid N at a residue corresponding to position 56 in SEQ ID NO: 14;
(vii) the amino acid S at a residue corresponding to position 58 in SEQ ID NO: 14;
(viii) the amino acid S at a residue corresponding to position 61 in SEQ ID NO: 14;
(ix) the amino acid V or L at a residue corresponding to position 63 in SEQ ID NO: 14;
(x) the amino acid G at a residue corresponding to position 95 in SEQ ID NO: 14;
(xi) the amino acid S at a residue corresponding to position 96 in SEQ ID NO: 14;
(xii) the amino acid I at a residue corresponding to position 103 in SEQ ID NO: 14;
(xiii) the amino acid S at a residue corresponding to position 116 in SEQ ID NO: 14;
(xiv) the amino acid I at a residue corresponding to position 129 in SEQ ID NO: 14;
(xv) the amino acid R at a residue corresponding to position 136 in SEQ ID NO: 14;
(xvi) the amino acid E at a residue corresponding to position 143 in SEQ ID NO: 14;
(xvii) the amino acid L at a residue corresponding to position 158 in SEQ ID NO: 14;
(xviii) the amino acid A at a residue corresponding to position 173 in SEQ ID NO: 14;
(xix) the amino acid A at a residue corresponding to position 181 in SEQ ID NO: 14;
(xx) the amino acid S at a residue corresponding to position 237 in SEQ ID NO: 14;
(xxi) the amino acid V at a residue corresponding to position 242 in SEQ ID NO: 14;
(xxii) the amino acid R at a residue corresponding to position 247 in SEQ ID NO: 14;
(xxiii) the amino acid M at a residue corresponding to position 257 in SEQ ID NO: 14;
(xxiv) the amino acid E at a residue corresponding to position 268 in SEQ ID NO: 14;
(xxv) the amino acid V at a residue corresponding to position 273 in SEQ ID NO: 14;
(xxvi) the amino acid R at a residue corresponding to position 296 in SEQ ID NO: 14;
(xxvii) the amino acid Q at a residue corresponding to position 302 in SEQ ID NO: 14;
(xxviii) the amino acid I at a residue corresponding to position 309 in SEQ ID NO: 14;
(xxix) the amino acid S at a residue corresponding to position 311 in SEQ ID NO: 14;
(xxx) the amino acid E at a residue corresponding to position 340 in SEQ ID NO: 14;
(xxxi) the amino acid Q at a residue corresponding to position 344 in SEQ ID NO: 14;
(xxxii) the amino acid L at a residue corresponding to position 345 in SEQ ID NO: 14;
(xxxiii) the amino acid I at a residue corresponding to position 351 in SEQ ID NO: 14;
(xxxiv) the amino acid F at a residue corresponding to position 354 in SEQ ID NO: 14;
(xxxv) the amino acid Y at a residue corresponding to position 360 in SEQ ID NO: 14;
(xxxvi) the amino acid D at a residue corresponding to position 361 in SEQ ID NO: 14;
(xxxvii) the amino acid T at a residue corresponding to position 363 in SEQ ID NO: 14;
(xxxviii) the amino acid Q at a residue corresponding to position 377 in SEQ ID NO: 14;
(xxxix) the amino acid N at a residue corresponding to position 378 in SEQ ID NO: 14;
(xl) the amino acid A at a residue corresponding to position 379 in SEQ ID NO: 14;
(xli) the amino acid S at a residue corresponding to position 382 in SEQ ID NO: 14;
(xlii) the amino acid V at a residue corresponding to position 396 in SEQ ID NO: 14;
(xliii) the amino acid V at a residue corresponding to position 411 in SEQ ID NO: 14;
(xliv) the amino acid D at a residue corresponding to position 424 in SEQ ID NO: 14;
(xlv) the amino acid K at a residue corresponding to position 425 in SEQ ID NO: 14;
(xlvi) the amino acid T at a residue corresponding to position 430 in SEQ ID NO: 14;
(xlvii) the amino acid I at a residue corresponding to position 442 in SEQ ID NO: 14;
(xlviii) the amino acid I or V at a residue corresponding to position 443 in SEQ ID NO: 14;
(xlix) the amino acid I at a residue corresponding to position 446 in SEQ ID NO: 14;
(l) the amino acid C at a residue corresponding to position 447 in SEQ ID NO: 14;
(li) the amino acid L at a residue corresponding to position 459 in SEQ ID NO: 14;
(lii) the amino acid I at a residue corresponding to position 462 in SEQ ID NO: 14;
(liii) the amino acid N at a residue corresponding to position 464 in SEQ ID NO: 14;
(liv) the amino acid I at a residue corresponding to position 465 in SEQ ID NO: 14;
(lv) the amino acid M at a residue corresponding to position 469 in SEQ ID NO: 14;
(lvi) the amino acid K at a residue corresponding to position 475 in SEQ ID NO: 14;
(lvii) the amino acid M at a residue corresponding to position 479 in SEQ ID NO: 14;
(lviii) the amino acid I at a residue corresponding to position 489 in SEQ ID NO: 14;
(lix) the amino acid I at a residue corresponding to position 491 in SEQ ID NO: 14;
(lx) the amino acid N at a residue corresponding to position 492 in SEQ ID NO: 14;
(lxi) the amino acid D at a residue corresponding to position 493 in SEQ ID NO: 14;
(lxii) the amino acid F or P at a residue corresponding to position 494 in SEQ ID NO: 14;
(lxiii) the amino acid N at a residue corresponding to position 496 in SEQ ID NO: 14;
(lxiv) the amino acid D at a residue corresponding to position 516 in SEQ ID NO: 14;
(lxv) the amino acid L at a residue corresponding to position 524 in SEQ ID NO: 14;
(lxvi) the amino acid D at a residue corresponding to position 528 in SEQ ID NO: 14;
(lxvii) the amino acid L or R at a residue corresponding to position 542 in SEQ ID NO: 14;
(lxviii) the amino acid R at a residue corresponding to position 543 in SEQ ID NO: 14; and/or
(lxix) the amino acid R at a residue corresponding to position 544 in SEQ ID NO: 14.

76. The host cell of any one of claims 68-74, wherein the TS comprises at least 2, 3, 4, 5, 6, 7, 8, 9, 10, 11, 12, 13, 14, 15, or 16 amino acid substitutions at residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459,462, 464, 465, 469, 475,479, 489, 491, 492, 493, 494,495, 496, 516, 524, 528, 542, 543, and/or 544 in SEQ ID NO: 14.

77. The host cell of any one of claims 67-75, wherein the TS comprises at least 2, 3, 4, 5, 6, 7, 8, 9, 10, 11, 12, 13, 14, 15, or 16 amino acid substitutions at residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14.

78. The host cell of claim 76, where the TS comprises relative to SEQ ID NO: 14:
(i) Q58S, V288L, and F345L;
(ii) R31Q, V52I, H56N, Q58S, M61S, I74T, N90V, A250P, S255V, F345L, Q475K, and T492N;
(iii) R31Q, H56N, I74T, N90V, H143E, A250P, S255V, Q475K, and T492N;
(iv) R31Q, H56N, I74T, N90V, A250P, S255V, L443I, Q475K, and T492N;
(v) H56N, M61S, N90V, A250D, S255V, V288L, Q475K, T492N, and A495E;
(vi) R31Q, H56N, I74T, N90V, K215R, A250P, S255V, Q475K, and T492N;
(vii) R31Q, P49A, H56N, Q58S, M61 S, I74T, N90V, H143E, A250P, S255V, V288L, F345L, Q475K, and T492N;
(viii) R31Q, A47T, H56N, 174T, N90V, A250P, S255V, Q475K, and T492N;
(ix) M61S, N90V, A250D, S255V, Q475K, T492N, A495E, and N498T;
(x) R31Q, H56N, M61S, I74T, N89H, N90V, S100A, H136R, E150Q, N196K, N211D, A250P, S255V, V288M, F345M, S382K, L443I, Q475K, and T492N;
(xi) R31Q, H56N, Q58S, M61S, I74T, N90V, H143E, A250P, S255V, V288L, T340E, F345L, E424D, Q475K, and T492N;
(xii) R31Q, H56N, I74T, S88L, N90V, A250P, S255V, Q475K, and T492N;
(xiii) R31Q, V52I, H56N, Q58S, M61S, I74T, N90V, H143E, A250P, S255V, V288L, F345L, Q475K, and T492N;
(xiv) R31Q, H56N, Q58S, M61S, 174T, N90V, H143E, A250P, S255V, V288L, T340E, F345L, E424D, Q475K, and T492N;
(xv) R31Q, H56N, Q58S, M61S, I74T, N90V, H143E, A250P, S255V, V288L, F345L, A411V, Q475K, and T492N;
(xvi) R31Q, V52L, H56N, Q58S, M61S, I74T, N90V, A250P, S255V, V288L, F345L, Q475K, and T492N;
(xvii) R31Q, K50L, H56N, I74T, N90V, A250P, S255V, Q475K, and T492N;
(xviii) R31Q, A47T, V52I, H56N, Q58S, M61S, 174T, N90V, H143E, A250P, S255V, V288L, T340E, F345L, Q475K, and T492N; or
(xix) R31Q, H56N, M61S, I74T, N89H, N90V, S100A, N196K, N211D, A250P, S255V, I257R, V288M, F345M, S382K, L443I, Q475K, and T492N.

79. The host cell of any one of claims 67-78, wherein the TS comprises a sequence that is at least 90%, at least 95%, at least 97%, at least 98%, or at least 99% identical to any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993.

80. The host cell of claim 79, wherein the TS comprises a sequence that is at least 90%, at least 95%, at least 97%, at least 98%, or at least 99% identical to any one of SEQ ID NOs: 698-716.

81. The host cell of claim 79, wherein the TS comprises the sequence of any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993.

82. A host cell that comprises a heterologous polynucleotide encoding a terminal synthase (TS), wherein the TS comprises a sequence that is at least 98% identical to SEQ ID NO: 39, and wherein the host cell is capable of producing a CBC-type cannabinoid.

83. The host cell of claim 82, wherein the CBC-type cannabinoid is CBCA and/or CBCVA.

84. The host cell of claim 82 or 83, wherein the TS is capable of producing more of a CBC-type cannabinoid than a control TS, wherein the control TS comprises the sequence of SEQ ID NO: 21.

85. The host cell of any one of claims 82-84, wherein the TS is capable of producing at least 0.05%, 0.075%, 0.1%, 0.5%, 0.75%, 1%, 5%, 10%, 20%, 30%, 40%, 50%, 60%, 70%, 80%, 90%, 120%, 150%, 170%, 200%, 240%, 290%, or 300/a more of a CBC-type cannabinoid than a control TS, wherein the control TS comprises the sequence of SEQ ID NO: 21.

86. The host cell of any one of claims 67-85, wherein the TS further comprises a first signal peptide.

87. The host cell of claim 86, wherein the first signal peptide comprises SEQ ID NO: 16 or a sequence that has no more than two amino acid substitutions, insertions, additions, or deletions relative to the sequence of SEQ ID NO: 16.

88. The host cell of claim 86 or 87, wherein the first signal peptide is located at the amino terminus of the TS.

89. The host cell of claim 87 or 88, wherein a methionine residue is added to the N-terminus of SEQ ID NO: 16.

90. The host cell of any one of claims 86-89, wherein the TS further comprises a second signal peptide.

91. The host cell of claim 90, wherein the second signal peptide comprises SEQ ID NO: 17 or a sequence that has no more than one amino acid substitution, insertion, addition, or deletion relative to the sequence of SEQ ID NO: 17.

92. The host cell of claim 90 or 91, wherein the second signal peptide is located at the carboxyl terminus of the TS.

93. The host cell of any one of claims 67-92, wherein the host cell further produces one or more of THCA, THCVA, CBDA and/or CBDVA.

94. The host cell of any one of claims 67-93, wherein the TS produces a higher ratio of CBCA:THCA, CBCA:CBDA, CBCVA:THCVA, and/or CBCVA:CBDVA than a control TS.

95. The host cell of claim 94, wherein the control TS is a TS comprising the sequence of SEQ ID NO: 21.

96. The host cell of any one of 67-95, wherein the TS has a higher product specificity for a THC-type cannabinoid than a control TS.

97. The host cell of claim 96, wherein the control TS is a TS comprising the sequence of SEQ ID NO: 21.

98. The host cell of any one of claims 1-97, wherein the host cell is a plant cell, an algal cell, a yeast cell, a bacterial cell, or an animal cell.

99. The host cell of claim 98, wherein the host cell is a yeast cell.

100. The host cell of claim 99, wherein the yeast cell is a Saccharomyces cell, a Yarrowia cell, a Komagataella cell, or a Pichia cell.

101. The host cell of claim 100, wherein the Saccharomyces cell is a Saccharomyces cerevisiae cell.

102. The host cell of claim 100, wherein the yeast cell is a Yarrowia cell.

103. The host cell of claim 98, wherein the host cell is a bacterial cell.

104. The host cell of claim 103, wherein the bacterial cell is an E. coli cell.

105. The host cell of any one of claims 98-104, wherein the host cell further comprises one or more heterologous polynucleotides encoding one or more of: an acyl activating enzyme (AAE), a polyketide synthase (PKS), a polyketide cyclase (PKC), a prenyltransferase (PT), and/or an additional terminal synthase (TS).

106. The host cell of claim 105, wherein the PKS is an olivetol synthase (OLS) or a divarinol synthase.

107. A method comprising culturing the host cell of any one of claims 1-106.

108. A method for producing a cannabinoid comprising contacting a CBG-type cannabinoid with a terminal synthase (TS), wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14.

109. A method for producing a cannabinoid comprising contacting a CBG-type cannabinoid with a terminal synthase (TS), wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129,136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14, wherein the TS does not comprise SEQ ID NO: 20, 21, 320 or 321, wherein the TS is capable of producing more of a THC-type cannabinoid than a control TS, and wherein the control TS comprises the sequence of SEQ ID NO: 284 or SEQ ID NO: 21.

110. A method for producing a cannabinoid comprising contacting a CBG-type cannabinoid with a terminal synthase (TS), wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13.

111. A method for producing a cannabinoid comprising contacting a CBG-type cannabinoid with a terminal synthase (TS), wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168, 171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13, wherein the TS is capable of producing more of a CBD-type cannabinoid than a control TS, and wherein the control TS comprises the sequence of SEQ ID NO: 136.

112. A method for producing a cannabinoid comprising contacting a CBG-type cannabinoid with a terminal synthase (TS), wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129, 136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14 and wherein the TS is capable of producing a CBC-type cannabinoid.

113. A method for producing a cannabinoid comprising contacting a CBG-type cannabinoid with a terminal synthase (TS), wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14, wherein the TS is capable of producing more of a CBC-type cannabinoid than a control TS, and wherein the control TS comprises the sequence of SEQ ID NO: 21.

114. The method of any one of claims 108-113, wherein contacting the CBG-type cannabinoid with the TS occurs in vitro.

115. The method of any one of claims 108-113, wherein contacting the CBG-type cannabinoid with the TS occurs in vivo.

116. The method of claim 115, wherein contacting the CBG-type cannabinoid with the TS occurs in a host cell.

117. A non-naturally occurring terminal synthase (TS), wherein relative to the sequence of SEQ ID NO: 14, the TS comprises an amino acid substitution at one or more residues corresponding to positions 36, 44, 47, 52, 58, 76, 85, 88, 89, 95, 129, 136, 150, 158, 181, 211, 237, 242, 247, 255, 267, 268, 273, 274, 288, 302, 309, 318, 329, 340, 344, 345, 351, 360, 361, 363, 379, 382, 396, 419, 424, 443, 459, 462, 464, 469, 479, 475, 491, 492, and/or 499 in SEQ ID NO: 14, and wherein the TS is capable of producing a THC-type cannabinoid.

118. The non-naturally occurring TS of claim 117, wherein relative to the sequence of SEQ ID NO: 14, the TS further comprises an amino acid substitution at one or more residues corresponding to positions 31, 40, 41, 46, 49, 51, 56, 59, 61, 63, 74, 90, 96, 100, 103, 116, 143, 173, 196, 250, 257, 290, 296, 311, 354, 377, 378, 411, 417, 446, 494, 495, 528, 542, 543 and/or 544 in SEQ ID NO: 14.

119. The non-naturally occurring TS of claim 118, wherein the TS comprises:
(i) the amino acid Q at a residue corresponding to position 31 in SEQ ID NO: 14;
(ii) the amino acid H or Q at a residue corresponding to position 36 in SEQ ID NO: 14;
(iii) the amino acid E or Q at a residue corresponding to position 40 in SEQ ID NO: 14;
(iv) the amino acid Y at a residue corresponding to position 41 in SEQ ID NO: 14;
(v) the amino acid T at a residue corresponding to position 44 in SEQ ID NO: 14,
(vi) the amino acid A or P at a residue corresponding to position 46 in SEQ ID NO: 14;
(vii) the amino acid T at a residue corresponding to position 47 in SEQ ID NO: 14;
(viii) the amino acid A at a residue corresponding to position 49 in SEQ ID NO: 14;
(ix) the amino acid F at a residue corresponding to position 51 in SEQ ID NO: 14;
(x) the amino acid I at a residue corresponding to position 52 in SEQ ID NO: 14;
(xi) the amino acid N at a residue corresponding to position 56 in SEQ ID NO: 14;
(xii) the amino acid P or S at a residue corresponding to position 58 in SEQ ID NO: 14;
(xiii) the amino acid F at a residue corresponding to position 59 in SEQ ID NO: 14;
(xiv) the amino acid S at a residue corresponding to position 61 in SEQ ID NO: 14;
(xv) the amino acid L or V at a residue corresponding to position 63 in SEQ ID NO: 14;
(xvi) the amino acid T at a residue corresponding to position 74 in SEQ ID NO: 14;
(xvii) the amino acid N at a residue corresponding to position 76 in SEQ ID NO: 14;
(xviii) the amino acid I at a residue corresponding to position 85 in SEQ ID NO: 14;
(xix) the amino acid L at a residue corresponding to position 88 in SEQ ID NO: 14;
(xx) the amino acid D, E, or H at a residue corresponding to position 89 in SEQ ID NO: 14;
(xxi) the amino acid E or V at a residue corresponding to position 90 in SEQ ID NO: 14;
(xxii) the amino acid G at a residue corresponding to position 95 in SEQ ID NO: 14;
(xxiii) the amino acid S at a residue corresponding to position 96 in SEQ ID NO: 14;
(xxiv) the amino acid A at a residue corresponding to position 100 in SEQ ID NO: 14;
(xxv) the amino acid I at a residue corresponding to position 103 in SEQ ID NO: 14;
(xxvi) the amino acid S at a residue corresponding to position 116 in SEQ ID NO: 14;
(xxvii) the amino acid I at a residue corresponding to position 129 in SEQ ID NO: 14;
(xxviii) the amino acid R at a residue corresponding to position 136 in SEQ ID NO: 14;
(xxix) the amino acid E at a residue corresponding to position 143 in SEQ ID NO: 14;
(xxx) the amino acid Q at a residue corresponding to position 150 in SEQ ID NO: 14;
(xxxi) the amino acid L at a residue corresponding to position 158 in SEQ ID NO: 14;
(xxxii) the amino acid A at a residue corresponding to position 173 in SEQ ID NO: 14;
(xxxiii) the amino acid A at a residue corresponding to position 181 in SEQ ID NO: 14;
(xxxiv) the amino acid K at a residue corresponding to position 196 in SEQ ID NO: 14;
(xxxv) the amino acid D at a residue corresponding to position 211 in SEQ ID NO: 14;
(xxxvi) the amino acid S at a residue corresponding to position 237 in SEQ ID NO: 14;
(xxxvii) the amino acid V at a residue corresponding to position 242 in SEQ ID NO: 14;
(xxxviii) the amino acid R at a residue corresponding to position 247 in SEQ ID NO: 14;
(xxxix) the amino acid D or P at a residue corresponding to position 250 in SEQ ID NO: 14;
(xl) the amino acid V at a residue corresponding to position 255 in SEQ ID NO: 14;
(xli) the amino acid M or R at a residue corresponding to position 257 in SEQ ID NO: 14;
(xlii) the amino acid N at a residue corresponding to position 267 in SEQ ID NO: 14;
(xliii) the amino acid E at a residue corresponding to position 268 in SEQ ID NO: 14;
(xliv) the amino acid V at a residue corresponding to position 273 in SEQ ID NO: 14;
(xlv) the amino acid H at a residue corresponding to position 274 in SEQ ID NO: 14;
(xlvi) the amino acid L, M, or T at a residue corresponding to position 288 in SEQ ID NO: 14;
(xlvii) the amino acid F at a residue corresponding to position 290 in SEQ ID NO: 14;
(xlviii) the amino acid R at a residue corresponding to position 296 in SEQ ID NO: 14;
(xlix) the amino acid Q at a residue corresponding to position 302 in SEQ ID NO: 14;
(l) the amino acid I at a residue corresponding to position 309 in SEQ ID NO: 14;
(li) the amino acid S at a residue corresponding to position 311 in SEQ ID NO: 14;
(lii) the amino acid L at a residue corresponding to position 318 in SEQ ID NO: 14;
(liii) the amino acid Q at a residue corresponding to position 329 in SEQ ID NO: 14;
(liv) the amino acid E at a residue corresponding to position 340 in SEQ ID NO: 14;
(lv) the amino acid Q at a residue corresponding to position 344 in SEQ ID NO: 14;
(lvi) the amino acid L or M at a residue corresponding to position 345 in SEQ ID NO: 14;
(lvii) the amino acid I at a residue corresponding to position 351 in SEQ ID NO: 14;
(lviii) the amino acid F at a residue corresponding to position 354 in SEQ ID NO: 14;
(lix) the amino acid Y at a residue corresponding to position 360 in SEQ ID NO: 14;
(lx) the amino acid D at a residue corresponding to position 361 in SEQ ID NO: 14;
(lxi) the amino acid T at a residue corresponding to position 363 in SEQ ID NO: 14;
(lxii) the amino acid Q at a residue corresponding to position 377 in SEQ ID NO: 14;
(lxiii) the amino acid N at a residue corresponding to position 378 in SEQ ID NO: 14;
(lxiv) the amino acid A at a residue corresponding to position 379 in SEQ ID NO: 14;
(lxv) the amino acid K at a residue corresponding to position 382 in SEQ ID NO: 14;
(lxvi) the amino acid V at a residue corresponding to position 396 in SEQ ID NO: 14;
(lxvii) the amino acid V at a residue corresponding to position 411 in SEQ ID NO: 14;
(lxviii) the amino acid V at a residue corresponding to position 417 in SEQ ID NO: 14;
(lxix) the amino acid F at a residue corresponding to position 419 in SEQ ID NO: 14;
(lxx) the amino acid D at a residue corresponding to position 424 in SEQ ID NO: 14;
(lxxi) the amino acid I at a residue corresponding to position 443 in SEQ ID NO: 14;
(lxxii) the amino acid I at a residue corresponding to position 446 in SEQ ID NO: 14;
(lxxiii) the amino acid L at a residue corresponding to position 459 in SEQ ID NO: 14;
(lxxiv) the amino acid I at a residue corresponding to position 462 in SEQ ID NO: 14;
(lxxv) the amino acid N at a residue corresponding to position 464 in SEQ ID NO: 14;
(lxxvi) the amino acid M at a residue corresponding to position 469 in SEQ ID NO: 14;
(lxxvii) the amino acid K at a residue corresponding to position 475 in SEQ ID NO: 14;
(lxxviii) the amino acid M at a residue corresponding to position 479 in SEQ ID NO: 14;
(lxxix) the amino acid M at a residue corresponding to position 491 in SEQ ID NO: 14;
(lxxx) the amino acid N at a residue corresponding to position 492 in SEQ ID NO: 14;
(lxxxi) the amino acid D, E, F, or P at a residue corresponding to position 494 in SEQ ID NO: 14;
(lxxxii) the amino acid E or K at a residue corresponding to position 495 in SEQ ID NO: 14;
(lxxxiii) the amino acid Q at a residue corresponding to position 499 in SEQ ID NO: 14;
(lxxxiv) the amino acid D at a residue corresponding to position 528 in SEQ ID NO: 14;
(lxxxv) the amino acid L or R at a residue corresponding to position 542 in SEQ ID NO: 14;
(lxxxvi) the amino acid R at a residue corresponding to position 543 in SEQ ID NO: 14; and/or
(lxxxvii) the amino acid R at a residue corresponding to position 544 in SEQ ID NO: 14.

120. The non-naturally occurring TS of claim 117, wherein the TS comprises:
(i) the amino acid H or Q at a residue corresponding to position 36 in SEQ ID NO: 14;
(ii) the amino acid T at a residue corresponding to position 44 in SEQ ID NO: 14;
(iii) the amino acid T at a residue corresponding to position 47 in SEQ ID NO: 14;
(iv) the amino acid I at a residue corresponding to position 52 in SEQ ID NO: 14;
(v) the amino acid P or S at a residue corresponding to position 58 in SEQ ID NO: 14;
(vi) the amino acid I at a residue corresponding to position 85 in SEQ ID NO: 14,
(vii) the amino acid L at a residue corresponding to position 88 in SEQ ID NO: 14;
(viii) the amino acid D, E, or H at a residue corresponding to position 89 in SEQ ID NO: 14;
(ix) the amino acid G at a residue corresponding to position 95 in SEQ ID NO: 14;
(x) the amino acid I at a residue corresponding to position 129 in SEQ ID NO: 14;
(xi) the amino acid R at a residue corresponding to position 136 in SEQ ID NO: 14;
(xii) the amino acid Q at a residue corresponding to position 150 in SEQ ID NO: 14;
(xiii) the amino acid L at a residue corresponding to position 158 in SEQ ID NO: 14;
(xiv) the amino acid A at a residue corresponding to position 181 in SEQ ID NO: 14;
(xv) the amino acid D at a residue corresponding to position 211 in SEQ ID NO: 14;
(xvi) the amino acid S at a residue corresponding to position 237 in SEQ ID NO: 14;
(xvii) the amino acid V at a residue corresponding to position 242 in SEQ ID NO: 14;
(xviii) the amino acid R at a residue corresponding to position 247 in SEQ ID NO: 14;
(xix) the amino acid V at a residue corresponding to position 255 in SEQ ID NO: 14;
(xx) the amino acid N at a residue corresponding to position 267 in SEQ ID NO: 14;
(xxi) the amino acid E at a residue corresponding to position 268 in SEQ ID NO: 14;
(xxii) the amino acid V at a residue corresponding to position 273 in SEQ ID NO: 14;
(xxiii) the amino acid H at a residue corresponding to position 274 in SEQ ID NO: 14;
(xxiv) the amino acid L, M, or T at a residue corresponding to position 288 in SEQ ID NO: 14;
(xxv) the amino acid Q at a residue corresponding to position 302 in SEQ ID NO: 14;
(xxvi) the amino acid I at a residue corresponding to position 309 in SEQ ID NO: 14;
(xxvii) the amino acid L at a residue corresponding to position 318 in SEQ ID NO: 14;
(xxviii) the amino acid Q at a residue corresponding to position 329 in SEQ ID NO: 14;
(xxix) the amino acid E at a residue corresponding to position 340 in SEQ ID NO: 14;
(xxx) the amino acid Q at a residue corresponding to position 344 in SEQ ID NO: 14;
(xxxi) the amino acid L or M at a residue corresponding to position 345 in SEQ ID NO: 14;
(xxxii) the amino acid I at a residue corresponding to position 351 in SEQ ID NO: 14;
(xxxiii) the amino acid Y at a residue corresponding to position 360 in SEQ ID NO: 14;
(xxxiv) the amino acid D at a residue corresponding to position 361 in SEQ ID NO: 14;
(xxxv) the amino acid T at a residue corresponding to position 363 in SEQ ID NO: 14;
(xxxvi) the amino acid A at a residue corresponding to position 379 in SEQ ID NO: 14;
(xxxvii) the amino acid K at a residue corresponding to position 382 in SEQ ID NO: 14;
(xxxviii) the amino acid V at a residue corresponding to position 396 in SEQ ID NO: 14;
(xxxix) the amino acid F at a residue corresponding to position 419 in SEQ ID NO: 14;
(xl) the amino acid D at a residue corresponding to position 424 in SEQ ID NO: 14;
(xli) the amino acid I at a residue corresponding to position 443 in SEQ ID NO: 14;
(xlii) the amino acid L at a residue corresponding to position 459 in SEQ ID NO: 14;
(xliii) the amino acid I at a residue corresponding to position 462 in SEQ ID NO: 14;
(xliv) the amino acid N at a residue corresponding to position 464 in SEQ ID NO: 14;
(xlv) the amino acid M at a residue corresponding to position 469 in SEQ ID NO: 14;
(xlvi) the amino acid M at a residue corresponding to position 479 in SEQ ID NO: 14;
(xlvii) the amino acid M at a residue corresponding to position 491 in SEQ ID NO: 14;
(xlviii) the amino acid N at a residue corresponding to position 492 in SEQ ID NO: 14; and/or
(xlix) the amino acid Q at a residue corresponding to position 499 in SEQ ID NO: 14.

121. The non-naturally occurring TS of claim 119, wherein the TS comprises relative to SEQ ID NO: 14:
(i) R31Q, H56N, Q58S, M61S, I74T, N90V, H143E, A250P, S255V, V288L, T340E, F345L, E424D, Q475K, T492N, and P542L;
(ii) R31Q, V52I, H56N, Q58S, M61S, 174T, N90V, A250P, S255V, V288L, F345L, Q475K, and T492N;
(iii) R31Q, A47T, V52I, H56N, Q58S, M61S, 174T, N90V, H143E, A250P, S255V, V288L, T340E, F345L, Q475K, and T492N;
(iv) H56N, Q58S, M61S, 174T, N90V, H143E, A250D, S255V, V288L, F345L, Q475K, T492N, and A495E;
(v) R31Q, H56N, Q58S, M61S, I74T, N90V, H143E, A250P, S255V, V288L, Q475K, and T492N;
(vi) R31Q, A47T, H56N, Q58S, M61S, 174T, N90V, H143E, A250P, S255V, V288L, T340E, F345L, E424D, Q475K, and T492N;
(vii) A47T, H56N, Q58S, M61S, I74T, N90V, A250D, S255V, F345L, E424D, Q475K, and T492N;
(viii) R31Q, A47T, H56N, Q58S, M61S, I74T, N90V, A250P, S255V, V288L, T340E, F345L, Q475K, and T492N;
(ix) R31Q, A47T, H56N, Q58S, M61S, I74T, N90V, H143E, A250P, S255V, V288L, T340E, F345L, Q475K, and T492N;
(x) A47T, H56N, Q58S, M61S, 174T, N90V, A250D, S255V, F345L, Q475K, and T492N;
(xi) R31Q, A47T, H56N, Q58S, M61S, I74T, N90V, H143E, A250P, S255V, T340E, F345L, Q475K, and T492N;
(xii) R31Q, H56N, Q58S, M61S, I74T, N90V, A250P, S255V, V288L, F345L, E424D, Q475K, and T492N;
(xiii) R3 1Q, A47T, H56N, Q58S, M61S, I74T, N90V, A250P, S255V, T340E, F345L, E424D, Q475K, and T492N;
(xiv) A47T, H56N, Q58S, M61S, I74T, N90V, A250D, S255V, V288L, F345L, Q475K, and T492N;
(xv) H56N, Q58S, M61S, I74T, N90V, H143E, A250D, S255V, V288L, F345L, Q475K, and T492N; or
(xvi) R31Q, V52I, H56N, M61S, I74T, N90V, A250P, S255V, F345L, Q475K, and T492N.

122. The non-naturally occurring TS of claim 120, wherein the TS comprises relative to SEQ ID NO: 14:
(iv) M61S, N90V, A250D, S255V, Q475K, T492N, and A495E;
(v) H56N, M61S, I74T, N90V, A250P, S255V, T492N, and H494E; or
(vi) R31Q, H56N, I74T, N90V, A250P, S255V, Q475K, T492N, H494E, and A495E.

123. The host cell of any one of claims 117-122, wherein the TS comprises a sequence that is at least 90%, at least 95%, at least 97%, at least 98%, at least 99% identical, or is 100% identical to any one of SEQ ID NOs: 138, 140, 141, 144, 155, 158, 164, 178, 198-200, 203, 285-289, 290-313, 474-487, 490-491, 499, 501-502, 504-505, 512, 515-517, 521-522, 524, 526-529, 532, 534-536, 538, 542-545, 548-605, 698-802, 804-811, 813-815, 820, 824, 826, 828-832, 834, 837-838, 845, 848, 850-851, 876, and 884-913.

124. A non-naturally occurring terminal synthase (TS), wherein relative to the sequence of SEQ ID NO: 13, the TS comprises an amino acid substitution at one or more residues corresponding to positions 79, 90, 106, 150, 166, 184, 211, 216, 230, 263, 273, 283, 290, 292, 319, 322, 339, 353, 380, 386, 397, 407, 416, 418, 441, 442, 446, 479, 450, 452, 454, 467, 481, 486, 504, and/or 512 in SEQ ID NO: 13, and wherein the TS is capable of producing a CBD-type cannabinoid.

125. The non-naturally occurring TS of claim 124, wherein relative to the sequence of SEQ ID NO: 13, the TS further comprises an amino acid substitution at one or more residues corresponding to positions 31, 47, 49, 50, 56, 57, 58, 69, 89, 95, 100, 103, 116, 124, 143, 162, 167, 168, 171, 172, 175, 180, 196, 213, 250, 287, 343, 344, 376, 377, 378, 394, 410, 414, 415, 445, 490, 492, 517 and/or 542 in SEQ ID NO: 13.

126. The non-naturally occurring TS of claim 125, wherein the TS comprises:
(i) the amino acid Q at a residue corresponding to position 31 in SEQ ID NO: 13;
(ii) the amino acid A at a residue corresponding to position 47 in SEQ ID NO: 13,
(iii) the amino acid P at a residue corresponding to position 49 in SEQ ID NO: 13;
(iv) the amino acid N at a residue corresponding to position 50 in SEQ ID NO: 13;
(v) the amino acid H at a residue corresponding to position 56 in SEQ ID NO: 13;
(vi) the amino acid D at a residue corresponding to position 57 in SEQ ID NO: 13;
(vii) the amino acid Q at a residue corresponding to position 58 in SEQ ID NO: 13;
(viii) the amino acid R or Q at a residue corresponding to position 69 in SEQ ID NO: 13;
(ix) the amino acid G at a residue corresponding to position 79 in SEQ ID NO: 13;
(x) the amino acid N, D, E, Q, or R at a residue corresponding to position 89 in SEQ ID NO: 13;
(xi) the amino acid C at a residue corresponding to position 90 in SEQ ID NO: 13;
(xii) the amino acid A at a residue corresponding to position 95 in SEQ ID NO: 13;
(xiii) the amino acid A at a residue corresponding to position 100 in SEQ ID NO: 13;
(xiv) the amino acid H at a residue corresponding to position 103 in SEQ ID NO: 13;
(xv) the amino acid E at a residue corresponding to position 106 in SEQ ID NO: 13;
(xvi) the amino acid A or G at a residue corresponding to position 116 in SEQ ID NO: 13;
(xvii) the amino acid N or M at a residue corresponding to position 124 in SEQ ID NO: 13;
(xviii) the amino acid E at a residue corresponding to position 143 in SEQ ID NO: 13;
(xix) the amino acid Q at a residue corresponding to position 150 in SEQ ID NO: 13;
(xx) the amino acid I at a residue corresponding to position 162 in SEQ ID NO: 13;
(xxi) the amino acid S at a residue corresponding to position 166 in SEQ ID NO: 13;
(xxii) the amino acid K at a residue corresponding to position 167 in SEQ ID NO: 13;
(xxiii) the amino acid T at a residue corresponding to position 168 in SEQ ID NO: 13;
(xxiv) the amino acid F at a residue corresponding to position 171 in SEQ ID NO: 13;
(xxv) the amino acid P at a residue corresponding to position 172 in SEQ ID NO: 13;
(xxvi) the amino acid F at a residue corresponding to position 175 in SEQ ID NO: 13;
(xxvii) the amino acid G at a residue corresponding to position 180 in SEQ ID NO: 13;
(xxviii) the amino acid F at a residue corresponding to position 184 in SEQ ID NO: 13;
(xxix) the amino acid K at a residue corresponding to position 196 in SEQ ID NO: 13;
(xxx) the amino acid D at a residue corresponding to position 211 in SEQ ID NO: 13;
(xxxi) the amino acid N at a residue corresponding to position 213 in SEQ ID NO: 13;
(xxxii) the amino acid L at a residue corresponding to position 216 in SEQ ID NO: 13;
(xxxiii) the amino acid I at a residue corresponding to position 230 in SEQ ID NO: 13;
(xxxiv) the amino acid R at a residue corresponding to position 250 in SEQ ID NO: 13;
(xxxv) the amino acid L at a residue corresponding to position 263 in SEQ ID NO: 13;
(xxxvi) the amino acid H at a residue corresponding to position 273 in SEQ ID NO: 13;
(xxxvii) the amino acid P at a residue corresponding to position 283 in SEQ ID NO: 13;
(xxxviii) the amino acid T at a residue corresponding to position 287 in SEQ ID NO: 13;
(xxxix) the amino acid M or A at a residue corresponding to position 290 in SEQ ID NO: 13;
(xl) the amino acid M at a residue corresponding to position 292 in SEQ ID NO: 13;
(xli) the amino acid D or N at a residue corresponding to position 319 in SEQ ID NO: 13;
(xlii) the amino acid E at a residue corresponding to position 322 in SEQ ID NO: 13;
(xliii) the amino acid E at a residue corresponding to position 339 in SEQ ID NO: 13;
(xliv) the amino acid E at a residue corresponding to position 343 in SEQ ID NO: 13;
(xlv) the amino acid M at a residue corresponding to position 344 in SEQ ID NO: 13;
(xlvi) the amino acid M at a residue corresponding to position 353 in SEQ ID NO: 13;
(xlvii) the amino acid L, Y, A, G, N, P, R, S, T, or V at a residue corresponding to position 376 in SEQ ID NO: 13;
(xlviii) the amino acid F, P, or R at a residue corresponding to position 377 in SEQ ID NO: 13;
(xlix) the amino acid K, R, S, or T at a residue corresponding to position 378 in SEQ ID NO: 13;
(l) the amino acid Y at a residue corresponding to position 380 in SEQ ID NO: 13;
(li) the amino acid F at a residue corresponding to position 386 in SEQ ID NO: 13;
(lii) the amino acid E at a residue corresponding to position 394 in SEQ ID NO: 13;
(liii) the amino acid E or K at a residue corresponding to position 397 in SEQ ID NO: 13;
(liv) the amino acid E at a residue corresponding to position 407 in SEQ ID NO: 13;
(lv) the amino acid T or V at a residue corresponding to position 410 in SEQ ID NO: 13;
(lvi) the amino acid I, L, M, T, or V at a residue corresponding to position 414 in SEQ ID NO: 13;
(lvii) the amino acid M at a residue corresponding to position 415 in SEQ ID NO: 13;
(lviii) the amino acid F, I, or M at a residue corresponding to position 416 in SEQ ID NO: 13;
(lix) the amino acid F at a residue corresponding to position 418 in SEQ ID NO: 13;
(lx) the amino acid S or T at a residue corresponding to position 441 in SEQ ID NO: 13;
(lxi) the amino acid I at a residue corresponding to position 442 in SEQ ID NO: 13;
(lxii) the amino acid V or A at a residue corresponding to position 445 in SEQ ID NO: 13;
(lxiii) the amino acid T or V at a residue corresponding to position 446 in SEQ ID NO: 13;
(lxiv) the amino acid S at a residue corresponding to position 450 in SEQ ID NO: 13;
(lxv) the amino acid T at a residue corresponding to position 452 in SEQ ID NO: 13;
(lxvi) the amino acid A at a residue corresponding to position 454 in SEQ ID NO: 13;
(lxvii) the amino acid Y at a residue corresponding to position 467 in SEQ ID NO: 13;
(lxviii) the amino acid S or T at a residue corresponding to position 479 in SEQ ID NO: 13;
(lxix) the amino acid I, M, V, or Y at a residue corresponding to position 481 in SEQ ID NO: 13;
(lxx) the amino acid V at a residue corresponding to position 486 in SEQ ID NO: 13;
(lxxi) the amino acid T at a residue corresponding to position 490 in SEQ ID NO: 13;
(lxxii) the amino acid N at a residue corresponding to position 492 in SEQ ID NO: 13;
(lxxiii) the amino acid Q at a residue corresponding to position 504 in SEQ ID NO: 13;
(lxxiv) the amino acid N at a residue corresponding to position 512 in SEQ ID NO: 13;
(lxxv) the amino acid D at a residue corresponding to position 527 in SEQ ID NO: 13; and/or
(lxxvi) the amino acid M at a residue corresponding to position 542 in SEQ ID NO: 13.

127. The non-naturally occurring TS of claim 124, wherein the TS comprises:
(i) the amino acid G at a residue corresponding to position 79 in SEQ ID NO: 13;
(ii) the amino acid C at a residue corresponding to position 90 in SEQ ID NO: 13,
(iii) the amino acid E at a residue corresponding to position 106 in SEQ ID NO: 13;
(iv) the amino acid Q at a residue corresponding to position 150 in SEQ ID NO: 13;
(v) the amino acid S at a residue corresponding to position 166 in SEQ ID NO: 13;
(vi) the amino acid D at a residue corresponding to position 211 in SEQ ID NO: 13;
(vii) the amino acid N at a residue corresponding to position 213 in SEQ ID NO: 13;
(viii) the amino acid L at a residue corresponding to position 216 in SEQ ID NO: 13;
(ix) the amino acid I at a residue corresponding to position 230 in SEQ ID NO: 13;
(x) the amino acid L at a residue corresponding to position 263 in SEQ ID NO: 13;
(xi) the amino acid H at a residue corresponding to position 273 in SEQ ID NO: 13;
(xii) the amino acid P at a residue corresponding to position 283 in SEQ ID NO: 13;
(xiii) the amino acid T at a residue corresponding to position 287 in SEQ ID NO: 13,
(xiv) the amino acid M or A at a residue corresponding to position 290 in SEQ ID NO: 13;
(xv) the amino acid M at a residue corresponding to position 292 in SEQ ID NO: 13;
(xvi) the amino acid D or N at a residue corresponding to position 319 in SEQ ID NO: 13;
(xvii) the amino acid E at a residue corresponding to position 322 in SEQ ID NO: 13;
(xviii) the amino acid E at a residue corresponding to position 339 in SEQ ID NO: 13;
(xix) the amino acid E at a residue corresponding to position 343 in SEQ ID NO: 13;
(xx) the amino acid M at a residue corresponding to position 344 in SEQ ID NO: 13;
(xxi) the amino acid M at a residue corresponding to position 353 in SEQ ID NO: 13;
(xxii) the amino acid Y at a residue corresponding to position 380 in SEQ ID NO: 13;
(xxiii) the amino acid F at a residue corresponding to position 386 in SEQ ID NO: 13;
(xxiv) the amino acid E at a residue corresponding to position 394 in SEQ ID NO: 13;
(xxv) the amino acid E or K at a residue corresponding to position 397 in SEQ ID NO: 13;
(xxvi) the amino acid E at a residue corresponding to position 407 in SEQ ID NO: 13;
(xxvii) the amino acid F, I, or M at a residue corresponding to position 416 in SEQ ID NO: 13;
(xxviii) the amino acid F at a residue corresponding to position 418 in SEQ ID NO: 13;
(xxix) the amino acid S or T at a residue corresponding to position 441 in SEQ ID NO: 13;
(xxx) the amino acid I at a residue corresponding to position 442 in SEQ ID NO: 13;
(xxxi) the amino acid T or V at a residue corresponding to position 446 in SEQ ID NO: 13;
(xxxii) the amino acid S at a residue corresponding to position 450 in SEQ ID NO: 13;
(xxxiii) the amino acid S or T at a residue corresponding to position 479 in SEQ ID NO: 13;
(xxxiv) the amino acid I, M, V, or Y at a residue corresponding to position 481 in SEQ ID NO: 13,
(xxxv) the amino acid V at a residue corresponding to position 486 in SEQ ID NO: 13;
(xxxvi) the amino acid T at a residue corresponding to position 490 in SEQ ID NO: 13;
(xxxvii) the amino acid Q at a residue corresponding to position 504 in SEQ ID NO: 13; and/or
(xxxviii) the amino acid N at a residue corresponding to position 512 in SEQ ID NO: 13.

128. The non-naturally occurring TS of claim 125, wherein the TS comprises relative to SEQ ID NO: 13:
(i) K50N, G95A, N196K, H213N, T339E, Q343E, L344M, and A414V;
(ii) G95A, Y175F, T339E, Q343E, and A414V;
(iii) G95A, S116A, T339E, Q343E, A414V, and N527D;
(iv) G95A, E150Q, V162L, C180G, N196K, N211D, N273H, T339E, Q343E, and A414V;
(v) G95A, T339E, Q343E, Q376V, and A414V;
(vi) K50N, G95A, S100A, E150Q, V162L, C180G, N196K, N211D, H213N, S322E, T339E, Q343E, L344M, A414V, E452T, and I504Q;
(vii) G95A, N196K, T339E, Q343E, and A414V;
(viii) 50N, G95A, V103H, H213N, T339E, Q343E, L344M, and A414V;
(ix) G95A, T339E, Q343E, Q376R, and A414V; or
(x) K50N, H213N, L230I, T339E, Q343E, and L344M.

129. The non-naturally occurring TS of claim 125, wherein the TS comprises relative to SEQ ID NO: 13:
(i) K50N, H213N, L230I, T339E, Q343E, and L344M;
(ii) S100A, T339E, and Q343E;
(iii) T339E, Q343E, L344M, and N527D;
(iv) K50N, V162I, C180G, N196K, N21 ID, H213N, T339E, Q343E, and L344M;
(v) K50N, E150Q, V1621, C180G, N196K, N211D, H213N, T339E, Q343E, and L344M;
(vi) S116A, H213N, T339E, Q343E, L344M, and N527D;
(vii) N196K, T339E, and Q343E;
(viii) K50N, E150Q, V1621, A172P, C180G, N196K, N211D, H213N, T339E, Q343E, and L344M;
(ix) V216L, T339E, and Q343E;
(x) S116A, H213N, T339E, Q343E, and N527D;
(xi) S116A, T339E, Q343E, and N527D; or
(xii) T339E, Q343E, and Q376P.

130. The non-naturally occurring TS of any one of claims 124-129, wherein the TS comprises a sequence that is at least 90%, at least 95%, at least 97%, at least 98%, at least 99% identical or is 100% identical to any one of SEQ ID NOs: 143, 149, 151-153, 156, 160, 163, 165, 166, 168, 170-172, 175-180, 182-197, 201, 204, 205, 207-225, 464-473, 478-480, 484-485, 487-489, 492-498, 500, 503, 506-548, 550, 551-552, 556, 558, 565, 567, 569-570, 572-578, 582, 584, 586, 588, 591, 593-595, 597, 600, 602, 604, 605, 718, 755, 784, 786, 790-792, 794, 795, 798, 800, 801, 803, 804, 806-810, 812-821, 823, 825, 827-836, 838, 839, 841-868, 870-874, 875-879, 881, 883, 913-932, 939-941, 944, 945, 946, and 948.

131. A non-naturally occurring TS, wherein relative to the sequence of SEQ ID NO: 14, the TS comprises an amino acid substitution at one or more residues corresponding to positions 41, 47, 49, 51, 52, 56, 58, 61, 63, 95, 96, 103, 116, 129,136, 143, 158, 173, 181, 237, 242, 247, 257, 268, 273, 296, 302, 309, 311, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 496, 516, 524, 528, 542, 543, and/or 544 in SEQ ID NO: 14, and wherein the TS is capable of producing a CBC-type cannabinoid.

132. The non-naturally occurring TS of claim 131, wherein relative to the sequence of SEQ ID NO: 14, the TS further comprises an amino acid substitution at one or more residues corresponding to positions 31, 40, 46, 74, 90, 255, 288, 290, 318, and/or 495 in SEQ ID NO: 14.

133. The non-naturally occurring TS of claim 132, wherein the TS comprises:
(i) the amino acid Q at a residue corresponding to position 31 in SEQ ID NO: 14;
(ii) the amino acid E at a residue corresponding to position 40 in SEQ ID NO: 14;
(iii) the amino acid Y at a residue corresponding to position 41 in SEQ ID NO: 14;
(iv) the amino acid P at a residue corresponding to position 46 in SEQ ID NO: 14;
(v) the amino acid T at a residue corresponding to position 47 in SEQ ID NO: 14;
(vi) the amino acid A at a residue corresponding to position 49 in SEQ ID NO: 14;
(vii) the amino acid F at a residue corresponding to position 51 in SEQ ID NO: 14;
(viii) the amino acid I at a residue corresponding to position 52 in SEQ ID NO: 14;
(ix) the amino acid N at a residue corresponding to position 56 in SEQ ID NO: 14;
(x) the amino acid S at a residue corresponding to position 58 in SEQ ID NO: 14;
(xi) the amino acid S at a residue corresponding to position 61 in SEQ ID NO: 14,
(xii) the amino acid V or L at a residue corresponding to position 63 in SEQ ID NO: 14;
(xiii) the amino acid T at a residue corresponding to position 74 in SEQ ID NO: 14,
(xiv) the amino acid V at a residue corresponding to position 90 in SEQ ID NO: 14;
(xv) the amino acid G at a residue corresponding to position 95 in SEQ ID NO: 14;
(xvi) the amino acid S at a residue corresponding to position 96 in SEQ ID NO: 14;
(xvii) the amino acid I at a residue corresponding to position 103 in SEQ ID NO: 14,
(xviii) the amino acid S at a residue corresponding to position 116 in SEQ ID NO: 14;
(xix) the amino acid I at a residue corresponding to position 129 in SEQ ID NO: 14;
(xx) the amino acid R at a residue corresponding to position 136 in SEQ ID NO: 14;
(xxi) the amino acid E at a residue corresponding to position 143 in SEQ ID NO: 14;
(xxii) the amino acid L at a residue corresponding to position 158 in SEQ ID NO: 14;
(xxiii) the amino acid A at a residue corresponding to position 173 in SEQ ID NO: 14;
(xxiv) the amino acid A at a residue corresponding to position 181 in SEQ ID NO: 14;
(xxv) the amino acid S at a residue corresponding to position 237 in SEQ ID NO: 14;
(xxvi) the amino acid V at a residue corresponding to position 242 in SEQ ID NO: 14;
(xxvii) the amino acid R at a residue corresponding to position 247 in SEQ ID NO: 14;
(xxviii) the amino acid V at a residue corresponding to position 255 in SEQ ID NO: 14;
(xxix) the amino acid M at a residue corresponding to position 257 in SEQ ID NO: 14;
(xxx) the amino acid E at a residue corresponding to position 268 in SEQ ID NO: 14;
(xxxi) the amino acid V at a residue corresponding to position 273 in SEQ ID NO: 14;
(xxxii) the amino acid L at a residue corresponding to position 288 in SEQ ID NO: 14;
(xxxiii) the amino acid F at a residue corresponding to position 290 in SEQ ID NO: 14;
(xxxiv) the amino acid R at a residue corresponding to position 296 in SEQ ID NO: 14;
(xxxv) the amino acid Q at a residue corresponding to position 302 in SEQ ID NO: 14;
(xxxvi) the amino acid I at a residue corresponding to position 309 in SEQ ID NO: 14;
(xxxvii) the amino acid S at a residue corresponding to position 311 in SEQ ID NO: 14;
(xxxviii) the amino acid L at a residue corresponding to position 318 in SEQ ID NO: 14;
(xxxix) the amino acid E at a residue corresponding to position 340 in SEQ ID NO: 14;
(xl) the amino acid Q at a residue corresponding to position 344 in SEQ ID NO: 14;
(xli) the amino acid L at a residue corresponding to position 345 in SEQ ID NO: 14;
(xlii) the amino acid I at a residue corresponding to position 351 in SEQ ID NO: 14;
(xliii) the amino acid F at a residue corresponding to position 354 in SEQ ID NO: 14;
(xliv) the amino acid Y at a residue corresponding to position 360 in SEQ ID NO: 14;
(xlv) the amino acid D at a residue corresponding to position 361 in SEQ ID NO: 14;
(xlvi) the amino acid T at a residue corresponding to position 363 in SEQ ID NO: 14;
(xlvii) the amino acid Q at a residue corresponding to position 377 in SEQ ID NO: 14;
(xlviii) the amino acid N at a residue corresponding to position 378 in SEQ ID NO: 14;
(xlix) the amino acid A at a residue corresponding to position 379 in SEQ ID NO: 14;
(l) the amino acid S at a residue corresponding to position 382 in SEQ ID NO: 14;
(li) the amino acid V at a residue corresponding to position 396 in SEQ ID NO: 14;
(lii) the amino acid V at a residue corresponding to position 411 in SEQ ID NO: 14;
(liii) the amino acid D at a residue corresponding to position 424 in SEQ ID NO: 14;
(liv) the amino acid K at a residue corresponding to position 425 in SEQ ID NO: 14;
(lv) the amino acid T at a residue corresponding to position 430 in SEQ ID NO: 14;
(lvi) the amino acid I at a residue corresponding to position 442 in SEQ ID NO: 14;
(lvii) the amino acid I or V at a residue corresponding to position 443 in SEQ ID NO: 14;
(lviii) the amino acid I at a residue corresponding to position 446 in SEQ ID NO: 14;
(lix) the amino acid C at a residue corresponding to position 447 in SEQ ID NO: 14;
(lx) the amino acid L at a residue corresponding to position 459 in SEQ ID NO: 14;
(lxi) the amino acid I at a residue corresponding to position 462 in SEQ ID NO: 14;
(lxii) the amino acid N at a residue corresponding to position 464 in SEQ ID NO: 14;
(lxiii) the amino acid I at a residue corresponding to position 465 in SEQ ID NO: 14;
(lxiv) the amino acid M at a residue corresponding to position 469 in SEQ ID NO: 14;
(lxv) the amino acid K at a residue corresponding to position 475 in SEQ ID NO: 14;
(lxvi) the amino acid M at a residue corresponding to position 479 in SEQ ID NO: 14;
(lxvii) the amino acid I at a residue corresponding to position 489 in SEQ ID NO: 14;
(lxviii) the amino acid I at a residue corresponding to position 491 in SEQ ID NO: 14;
(lxix) the amino acid N at a residue corresponding to position 492 in SEQ ID NO: 14;
(lxx) the amino acid D at a residue corresponding to position 493 in SEQ ID NO: 14;
(lxxi) the amino acid F or P at a residue corresponding to position 494 in SEQ ID NO: 14;
(lxxii) the amino acid E or K at a residue corresponding to position 495 in SEQ ID NO: 14;
(lxxiii) the amino acid N at a residue corresponding to position 496 in SEQ ID NO: 14;
(lxxiv) the amino acid D at a residue corresponding to position 516 in SEQ ID NO: 14;
(lxxv) the amino acid L at a residue corresponding to position 524 in SEQ ID NO: 14;
(lxxvi) the amino acid D at a residue corresponding to position 528 in SEQ ID NO: 14;
(lxxvii) the amino acid L or R at a residue corresponding to position 542 in SEQ ID NO: 14;
(lxxviii) the amino acid R at a residue corresponding to position 543 in SEQ ID NO: 14; and/or
(lxxix) the amino acid R at a residue corresponding to position 544 in SEQ ID NO: 14.

134. The non-naturally occurring TS of claim 131, wherein the TS comprises:
(i) the amino acid Y at a residue corresponding to position 41 in SEQ ID NO: 14;
(ii) the amino acid T at a residue corresponding to position 47 in SEQ ID NO: 14;
(iii) the amino acid A at a residue corresponding to position 49 in SEQ ID NO: 14;
(iv) the amino acid F at a residue corresponding to position 51 in SEQ ID NO: 14;
(v) the amino acid I at a residue corresponding to position 52 in SEQ ID NO: 14;
(vi) the amino acid N at a residue corresponding to position 56 in SEQ ID NO: 14;
(vii) the amino acid S at a residue corresponding to position 58 in SEQ ID NO: 14;
(viii) the amino acid S at a residue corresponding to position 61 in SEQ ID NO: 14;
(ix) the amino acid V or L at a residue corresponding to position 63 in SEQ ID NO: 14;
(x) the amino acid G at a residue corresponding to position 95 in SEQ ID NO: 14;
(xi) the amino acid S at a residue corresponding to position 96 in SEQ ID NO: 14;
(xii) the amino acid I at a residue corresponding to position 103 in SEQ ID NO: 14;
(xiii) the amino acid S at a residue corresponding to position 116 in SEQ ID NO: 14;
(xiv) the amino acid I at a residue corresponding to position 129 in SEQ ID NO: 14;
(xv) the amino acid R at a residue corresponding to position 136 in SEQ ID NO: 14;
(xvi) the amino acid E at a residue corresponding to position 143 in SEQ ID NO: 14;
(xvii) the amino acid L at a residue corresponding to position 158 in SEQ ID NO: 14;
(xviii) the amino acid A at a residue corresponding to position 173 in SEQ ID NO: 14;
(xix) the amino acid A at a residue corresponding to position 181 in SEQ ID NO: 14;
(xx) the amino acid S at a residue corresponding to position 237 in SEQ ID NO: 14;
(xxi) the amino acid V at a residue corresponding to position 242 in SEQ ID NO: 14;
(xxii) the amino acid R at a residue corresponding to position 247 in SEQ ID NO: 14;
(xxiii) the amino acid M at a residue corresponding to position 257 in SEQ ID NO: 14;
(xxiv) the amino acid E at a residue corresponding to position 268 in SEQ ID NO: 14;
(xxv) the amino acid V at a residue corresponding to position 273 in SEQ ID NO: 14;
(xxvi) the amino acid R at a residue corresponding to position 296 in SEQ ID NO: 14;
(xxvii) the amino acid Q at a residue corresponding to position 302 in SEQ ID NO: 14;
(xxviii) the amino acid I at a residue corresponding to position 309 in SEQ ID NO: 14;
(xxix) the amino acid S at a residue corresponding to position 311 in SEQ ID NO: 14;
(xxx) the amino acid E at a residue corresponding to position 340 in SEQ ID NO: 14;
(xxxi) the amino acid Q at a residue corresponding to position 344 in SEQ ID NO: 14;
(xxxii) the amino acid L at a residue corresponding to position 345 in SEQ ID NO: 14;
(xxxiii) the amino acid I at a residue corresponding to position 351 in SEQ ID NO: 14;
(xxxiv) the amino acid F at a residue corresponding to position 354 in SEQ ID NO: 14;
(xxxv) the amino acid Y at a residue corresponding to position 360 in SEQ ID NO: 14;
(xxxvi) the amino acid D at a residue corresponding to position 361 in SEQ ID NO: 14;
(xxxvii) the amino acid T at a residue corresponding to position 363 in SEQ ID NO: 14;
(xxxviii) the amino acid Q at a residue corresponding to position 377 in SEQ ID NO: 14;
(xxxix) the amino acid N at a residue corresponding to position 378 in SEQ ID NO: 14;
(xl) the amino acid A at a residue corresponding to position 379 in SEQ ID NO: 14;
(xli) the amino acid S at a residue corresponding to position 382 in SEQ ID NO: 14;
(xlii) the amino acid V at a residue corresponding to position 396 in SEQ ID NO: 14;
(xliii) the amino acid V at a residue corresponding to position 411 in SEQ ID NO: 14;
(xliv) the amino acid D at a residue corresponding to position 424 in SEQ ID NO: 14;
(xlv) the amino acid K at a residue corresponding to position 425 in SEQ ID NO: 14;
(xlvi) the amino acid T at a residue corresponding to position 430 in SEQ ID NO: 14;
(xlvii) the amino acid I at a residue corresponding to position 442 in SEQ ID NO: 14;
(xlviii) the amino acid I or V at a residue corresponding to position 443 in SEQ ID NO: 14;
(xlix) the amino acid I at a residue corresponding to position 446 in SEQ ID NO: 14;
(l) the amino acid C at a residue corresponding to position 447 in SEQ ID NO: 14;
(li) the amino acid L at a residue corresponding to position 459 in SEQ ID NO: 14;
(lii) the amino acid I at a residue corresponding to position 462 in SEQ ID NO: 14;
(liii) the amino acid N at a residue corresponding to position 464 in SEQ ID NO: 14;
(liv) the amino acid I at a residue corresponding to position 465 in SEQ ID NO: 14;
(lv) the amino acid M at a residue corresponding to position 469 in SEQ ID NO: 14;
(lvi) the amino acid K at a residue corresponding to position 475 in SEQ ID NO: 14;
(lvii) the amino acid M at a residue corresponding to position 479 in SEQ ID NO: 14;
(lviii) the amino acid I at a residue corresponding to position 489 in SEQ ID NO: 14;
(lix) the amino acid I at a residue corresponding to position 491 in SEQ ID NO: 14;
(lx) the amino acid N at a residue corresponding to position 492 in SEQ ID NO: 14;
(lxi) the amino acid D at a residue corresponding to position 493 in SEQ ID NO: 14;
(lxii) the amino acid F or P at a residue corresponding to position 494 in SEQ ID NO: 14;
(lxiii) the amino acid N at a residue corresponding to position 496 in SEQ ID NO: 14;
(lxiv) the amino acid D at a residue corresponding to position 516 in SEQ ID NO: 14;
(lxv) the amino acid L at a residue corresponding to position 524 in SEQ ID NO: 14;
(lxvi) the amino acid D at a residue corresponding to position 528 in SEQ ID NO: 14;
(lxvii) the amino acid L or R at a residue corresponding to position 542 in SEQ ID NO: 14;
(lxviii) the amino acid R at a residue corresponding to position 543 in SEQ ID NO: 14; and/or
(lxix) the amino acid R at a residue corresponding to position 544 in SEQ ID NO: 14.

135. The non-naturally occurring TS of claim 132, where the TS comprises relative to SEQ ID NO: 14:
(i) Q58S, V288L, and F345L;
(ii) R31Q, V52I, H56N, Q58S, M61S, I74T, N90V, A250P, S255V, F345L, Q475K, and T492N;
(iii) R31Q, H56N, I74T, N90V, H143E, A250P, S255V, Q475K, and T492N;
(iv) R31Q, H56N, I74T, N90V, A250P, S255V, L443I, Q475K, and T492N;
(v) H56N, M61S, N90V, A250D, S255V, V288L, Q475K, T492N, and A495E;
(vi) R31Q, H56N, I74T, N90V, K215R, A250P, S255V, Q475K, and T492N;
(vii) R31Q, P49A, H56N, Q58S, M61S, 174T, N90V, H143E, A250P, S255V, V288L, F345L, Q475K, and T492N;
(viii) R31Q, A47T, H56N, 174T, N90V, A250P, S255V, Q475K, and T492N;
(ix) M61S, N90V, A250D, S255V, Q475K, T492N, A495E, and N498T;
(x) R31Q, H56N, M61S, 174T, N89H, N90V, S100A, H136R, E150Q, N196K, N21 ID, A250P, S255V, V288M, F345M, S382K, L443L, Q475K, and T492N;
(xi) R31Q, H56N, Q58S, M61S, I74T, N90V, H143E, A250P, S255V, V288L, T340E, F345L, E424D, Q475K, and T492N;
(xii) R31Q, H56N, I74T, S88L, N90V, A250P, S255V, Q475K, and T492N;
(xiii) R31Q, V52I, H56N, Q58S, M61S, I74T, N90V, H143E, A250P, S255V, V288L, F345L, Q475K, and T492N;
(xiv) R31Q, H56N, Q58S, M61S, 174T, N90V, H143E, A250P, S255V, V288L, T340E, F345L, E424D, Q475K, and T492N;
(xv) R31Q, H56N, Q58S, M61S, I74T, N90V, H143E, A250P, S255V, V288L, F345L, A411V, Q475K, and T492N;
(xvi) R31Q, V52I, H56N, Q58S, M61S, I74T, N90V, A250P, S255V, V288L, F345L, Q475K, and T492N;
(xvii) R31Q, K50L, H56N, I74T, N90V, A250P, S255V, Q475K, and T492N;
(xviii) R31Q, A47T, V52I, H56N, Q58S, M61S, I74T, N90V, H143E, A250P, S255V, V288L, T340E, F345L, Q475K, and T492N; or
(xix) R31Q, H56N, M61S, 174T, N89H, N90V, S100A, N196K, N211D, A250P, S255V, I257R, V288M, F345M, S382K, L443I, Q475K, and T492N.

136. The non-naturally occurring TS of any one of claims 131-135, wherein the TS comprises a sequence that is at least 90%, at least 95% at least 97%, at least 98%, at least 99%4 identical or is 100%/o identical to any one of SEQ ID NOs: 137-140, 142-143, 145-150, 154, 157, 159, 161, 162, 164, 167, 169, 173, 174, 177-193, 195, 196, 199, 204-206, 464-466, 488, 489, 492-498, 500, 502, 503, 506, 507-548, 550, 551, 552, 565, 574, 595, 597, 602, 698-882, and 993.

137. A non-naturally occurring nucleic acid encoding a terminal synthase (TS), wherein the non-naturally occurring nucleic acid comprises a sequence that is at least 90%, at least 95% at least 97%, at least 98%, at least 99% identical to any one of SEQ ID NOs: 46-134, 194-222, 322-463, 954-1189, 1195-1197, 1201, 1202, and 1204.

138. The non-naturally occurring nucleic acid of claim 137, wherein the non-naturally occurring nucleic acid comprises the sequence of any one of SEQ ID NOs: 46-134, 194-222, 322-463, 954-1189, 1195-1197, 1201, 1202, and 1204.

139. A vector comprising the non-naturally occurring nucleic acid of claim 137 or 138.

140. An expression cassette comprising the non-naturally occurring nucleic acid of claim 137 or 138.

141. A host cell transformed with the non-naturally occurring nucleic acid of claim 137 or 138, the vector of claim 139, or the expression cassette of claim 140.

142. A bioreactor for producing a cannabinoid, wherein the bioreactor contains a CBG-type cannabinoid and a terminal synthase (TS), wherein relative to the sequence of SEQ ID NO: 14, the TS comprises an amino acid substitution at one or more residues corresponding to positions 31, 36, 40, 41, 44, 46, 47, 49, 51, 52, 56, 58, 59, 61, 63, 74, 76, 85, 88, 89, 90, 95, 96, 100, 103, 116, 129, 136, 143, 150, 158, 173, 181, 196, 211, 237, 242, 247, 250, 255, 257, 267, 268, 273, 274, 288, 290, 296, 302, 309, 311, 318, 329, 340, 344, 345, 351, 354, 360, 361, 363, 377, 378, 379, 382, 396, 411, 417, 419, 424, 443, 446, 459, 462, 464, 469, 479, 475, 491, 492, 494, 495, 499, 528, 542, 543, and/or 544 in SEQ ID NO: 14, wherein the TS does not comprise the sequence of SEQ ID NO: 20, 21, 320 or 321.

143. A bioreactor for producing a cannabinoid, wherein the bioreactor contains a CBG-type cannabinoid and a terminal synthase (TS), wherein relative to the sequence of SEQ ID NO: 13, the TS comprises an amino acid substitution at one or more residues corresponding to positions 31, 47, 49, 50, 56, 57, 58, 69, 79, 89, 90, 95, 100, 103, 106, 116, 124, 143, 150, 162, 166, 167, 168, 171, 172, 175, 180, 184, 196, 211, 213, 216, 230, 250, 263, 273, 283, 287, 290, 292, 319, 322, 339, 343, 344, 353, 376, 377, 378, 380, 386, 394, 397, 407, 410, 414, 415, 416, 418, 441, 442, 445, 446, 479, 450, 452, 454, 467, 481, 486, 490, 492, 504, 512, 527 and/or 542 in SEQ ID NO: 13.

144. A bioreactor for producing a cannabinoid, wherein the bioreactor contains a CBG-type cannabinoid and a terminal synthase (TS), wherein relative to the sequence of SEQ ID NO: 14, the TS comprises an amino acid substitution at one or more residues corresponding to positions 31, 40, 41, 46, 47, 49, 51, 52, 56, 58, 61, 63, 74, 90, 95, 96, 103, 116, 129, 136, 143, 158, 173, 181, 237, 242, 247, 255, 257, 268, 273, 288, 290, 296, 302, 309, 311, 318, 340, 344, 345, 351, 354, 360, 361, 363, 377, 378, 379, 382, 396, 411, 424, 425, 430, 442, 443, 446, 447, 459, 462, 464, 465, 469, 475, 479, 489, 491, 492, 493, 494, 495, 496, 516, 524, 528, 542, 543, and/or 544 in SEQ ID NO: 14, wherein the TS is capable of producing more of a CBC-type cannabinoid than a control TS, and wherein the control TS comprises the sequence of SEQ ID NO: 21.

145. A bioreactor for producing a cannabinoid, wherein the bioreactor contains a CBG-type cannabinoid and a host cell of any one of claims 1-123.
